(19) United States
(12) Patent Application Publication
(10) Pub. No.: US 2022/0402948 A1
(43) Pub. Date: Dec. 22, 2022

(54) EGFR INHIBITOR, COMPOSITION AND PREPARATION METHOD THEREFOR

(71) Applicant: BETTA PHARMACEUTICALS CO., LTD., Hangzhou, Zhejiang (CN)

(72) Inventors: Xiangyong LIU, Beijing (CN); Changyong QIU, Beijing (CN); Haitong SHENG, Beijing (CN); Mengqiang LIU, Beijing (CN); Qichao SHEN, Beijing (CN); Guolong DU, Beijing (CN); Xiaodong SONG, Beijing (CN); Lieming DING, Hangzhou, Zhejiang (CN); Jiabing WANG, Beijing (CN)

(21) Appl. No.: 17/763,633

(22) PCT Filed: Sep. 25, 2020
(86) PCT No.: PCT/CN2020/117630

§ 371 Date: Mar. 24, 2022

(30) Foreign Application Priority Data
Sep. 26, 2019 (CN) 201910929907.9
Mar. 5, 2020 (CN) 202010149132.6

(51) Int. Cl. C07F 9/53

(52) U.S. Cl. CPC C07F 9/53

(57) ABSTRACT

The present invention relates to the compounds of Formula I, methods of using these compounds as EGFR inhibitors, and pharmaceutical compositions comprising compounds thereof. The compounds are used for treating, preventing or ameliorating diseases or disorders such as cancer or infections.

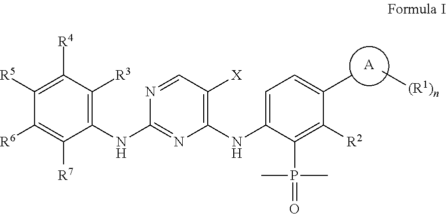

### FIELD OF THE INVENTION

The present invention is concerned with pharmaceutically active compounds, deuterated compounds (hydrogen substituted with deuterium) and pharmaceutically acceptable salts thereof, which can be used to treat or prevent diseases or medical conditions mediated by certain mutant forms of epidermal growth factor receptor (For example, L858R activating mutant, exon 19 deletion activating mutant, T790M resistant mutant and C797S resistant mutant). The present invention also relates to a pharmaceutical composition comprising the compound, and a method of using the compound, deuterated compound and their relating salts to treat diseases mediated by various forms of EGFR mutants.

### BACKGROUND OF THE INVENTION

Epidermal growth factor receptor (EGFR) is a kind of transmembrane glycoprotein belonging to the ErbB family of tyrosine kinase receptors. Activation of EGFR leads to autophosphorylation of receptor tyrosine kinases, which are involved in the cascade of downstream signal transduction pathway that regulate cell proliferation, differentiation, and survival. EGFR is abnormally activated by various mechanisms, such as receptor overexpression, mutation, ligand-dependent receptor dimerization, ligand-independent activation, and is related to the development of a variety of human cancers.

EGFR inhibition is one of the key targets of cancer therapy. Despite the rapid development of previous generations of EGFR-Tkis, the problem of drug resistance has also emerged with the development of drugs. Most drug resistance is due to the T790M mutation of the ATP receptor. Recently developed third-generation irreversible inhibitors for T790M, such as osimertinib, have good activity on inhibitory, but drug resistance will inevitably appear. Most drug resistance is due to the T790M mutation of the ATP receptor. Recently developed third-generation irreversible inhibitors for T790M, such as osimertinib, have good activity on inhibitory, but drug resistance will inevitably appear. C797S mutation is a kind of missense mutation in which serine replaces cysteine at site 797 of exon 20 of EGFR, and C797S is located in the tyrosine kinase region of EGFR. C797S mutation prevents osimertinib from forming covalent bonds in the ATP binding domain, thus losing its inhibitory effect on EGFR activation and resulting in drug resistance.

Early patent applications WO2018108064, WO2018115218, WO2018181777 disclosed a series of fourth-generation EGFR inhibitors, but there is still a need for EGFR C797S inhibitors with stronger activity. In the invention, the applicant discovers a kind of small molecules that can be used as the fourth-generation EGFR inhibitor, whose activity can be used to treat cancer and/or infectious diseases. These small molecules are expected to be used as drugs with stability, solubility, bioavailability, therapeutic indices and toxicity values, which are essential to develop effective drugs for human health.

### SUMMARY OF INVENTION

The present invention relates to compounds capable of inhibiting EGFR which can be useful in the treatment of cancers and infectious diseases.

The first purpose of the invention is to provide a compound as shown in Formula I, or stereoisomer, tautomer, deuterated compound, pharmaceutically acceptable salt, prodrug, chelate, non-covalent complex or solvate thereof

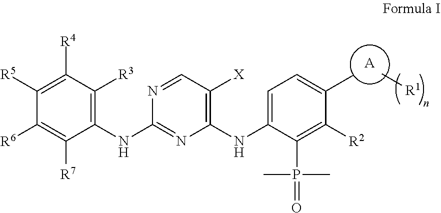

wherein.

X is halogen;

ring A is a 5- to 10-membered aromatic ring; the 5- to 10-membered aromatic ring is a 5- to 10-membered aryl or a 5- to 10-membered heteroaryl; R¹ is selected from halogen, CN, OH, —NRR⁸, oxo, aryl, heteroaryl, —C₁₋₆ alkyl, haloalkyl, —C₁₋₆ alkoxy, —(CH₂)ₚ(O(CH₂)_{q})ᵣCH₃, —C₃₋₆ cycloalkyl, 3- to 6-membered heterocyclyl, —C₁₋₆ alkyl substituted with a 3- to 7-membered heterocyclyl and

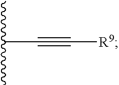

R² is selected from H, halogen, CN, NO₂, OH, —NRR⁸, —C₁₋₆ alkyl, —C₁₋₆ alkoxy, —(CH₂)ₚ(O(CH₂)_{q})ᵣCH₃, —C₃₋₄ cycloalkyl, 3- to 6-membered heterocyclyl; the —C₁₋₆ alkyl, —C₁₋₆ alkoxy, 3- to 6-membered heterocyclyl are optionally substituted or not substituted with halogen, OH, NH₂, aryl, heteroaryl, —C₁₋₆alkyl, —C₃₋₆ cycloalkyl;

R, R⁸ and R⁹ are independently selected from H, CN, —C₁₋₆ alkyl, —C₃₋₆ cycloalkyl and 3- to 6-membered heterocyclyl, wherein the —C₁₋₆ alkyl, —C₃₋₆ cycloalkyl and 3- to 6-membered heterocyclyl are optionally substituted or not substituted with halogen, OH, NH₂, aryl, heteroaryl, —C₁₋₆ alkyl, —C₃₋₆ cycloalkyl, 3- to 6-membered heterocyclyl;

R³, R⁴ and R⁷ are independently selected from H, halogen, CN, —NR¹⁰R¹¹, OH, —C₁₋₆ alkyl, —C₁₋₆ alkoxy, —(CH₂)ₚ(O(CH₂)_{q})ᵣCH₃, —C₃₋₆ cycloalkyl and 3- to 6-membered heterocyclyl, wherein the —C₁₋₆ alkyl, —C₁₋₆ alkoxy, —C₃₋₆ cycloalkyl and 3- to 6-membered heterocyclyl are optionally substituted or not substituted with halogen, OH, NH₂, —C₁₋₆ alkyl; and R³, R⁴ and R⁷ are not H at the same time;

R¹⁰ and R¹¹ are independently selected from H, —C₁₋₆ alkyl and —C_{M} cycloalkyl;

R⁵ and R⁶ are independently selected from H, halogen, CN, NO₂, OH, —NR¹²R¹³, —C₁₋₆ alkyl, aryl, heteroaryl, —C₁₋₆ alkoxy, —C₃₋₆ cycloalkyl, 3- to 6-membered heterocyclyl, —(CH₂)ₛCH═CHR¹⁴; the —C₁₋₆ alkyl, aryl, heteroaryl, —C₁₋₆ alkoxy, —C₃₋₆ cycloalkyl and 3- to 6-membered heterocyclyl are optionally substituted or unsubstituted with halogen. OH, NH₂, —C₁₋₆ alkoxy, —C₃₋₆ cycloalkyl, substituted C₃₋₆ cycloalkyl, 3- to 6-membered heterocyclyl and substituted 3- to 6-membered heterocyclyl, aryl, —C₁₋₆ alkanoyl and heteroaryl; and R⁵ and R⁶ are not H at the same time;

R¹². R¹³ and R¹⁴ are independently selected from H, —C₁₋₆ alkyl and —C₃₋₆ cycloalkyl;

n is any integer selected from 0 to 4;

p is any integer selected from 1 to 3;

q is any integer selected from 0 to 3;

r is any integer selected from 1 to 3;

s is any integer selected from 0 to 3.

In some embodiments, X is fluorine, chlorine, and bromine.

In some embodiments, ring A is a 5- to 10-membered aryl or a 5- to 10-membered heteroaryl, wherein the 5- to 10-membered heteroaryl contains 1, 2 or 3 heteroatoms selected from N or S.

In some embodiments, ring A is

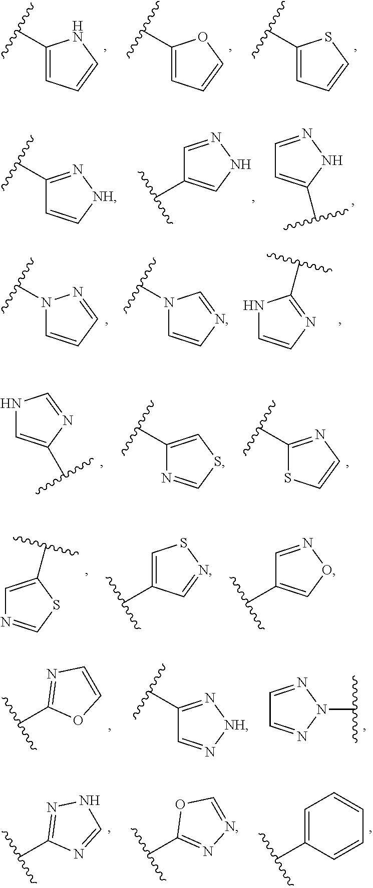

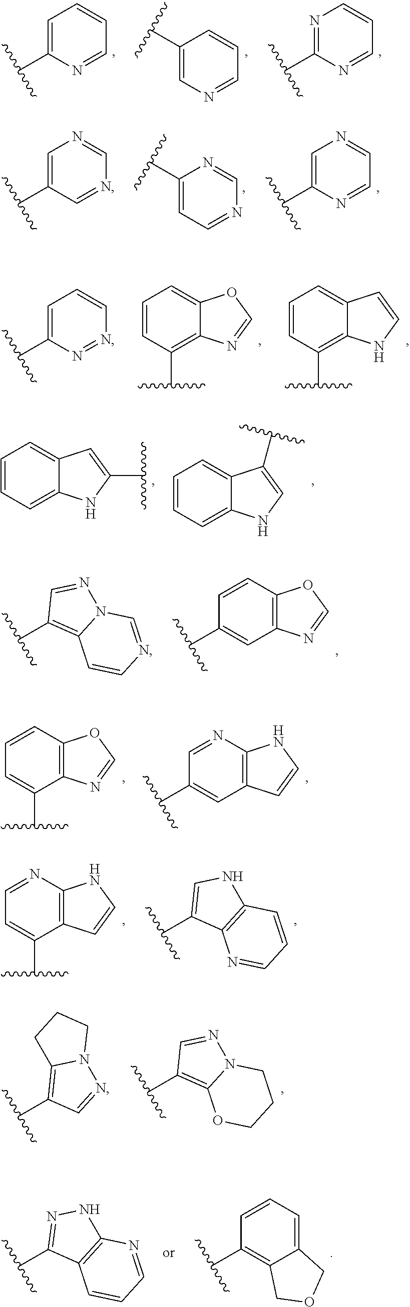

In some embodiments, R¹ is selected from halogen, CN, OH, —NH₂, —N(CH₃)₂, —C₁₋₆ alkyl, haloalkyl, —C₁₋₆ alkoxy, —(CH₂)ₚ(O(CH₂)_{q})ᵣCH₃, —C₃₋₆ cycloalkyl, —C₁₋₆ alkyl substituted with 3- to 7-membered heterocyclyl and

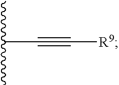

the above mentioned definitions of p, q, r, s and R⁹ are described in any embodiments of the invention.

In certain embodiments, R¹ is selected from halogen, CN, OH, —NH₂, —N(CH₃)₂, methyl, methoxy, —CF₃, methoxymethyl, H, oxo, —F, —Cl, —CHF₂,

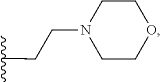

ethoxy, cyclopropyl, ethyl, isopropyl, alkynyl.

In some embodiments, R² is selected from H, halogen, —C₁₋₆ alkyl or —C₁₋₆ alkoxy.

In some embodiments, R² is selected from H, —F, methyl or methoxy.

In some embodiments, R³, R⁴ and R⁷ are independently selected from H, halogen, CN, OH, methyl, ethyl, propyl, isopropyl, methoxy, ethoxy, —OCH₂CF₃ and cyclic propyl; and R³, R⁴ and R⁷ are not H at the same time.

In some embodiments, R³ is selected from H or methoxy.

In some embodiments, R⁴ is selected from H, methoxy, ethoxy or —OCH₂CF₃.

In some embodiments, R⁷ is selected from H, methyl or cyclopropyl.

In some embodiments, R⁵ is selected from —NR¹²R¹¹, —C₁₋₆ alkyl, aryl, heteroaryl, —C₁₋₆ alkoxy, —C₁₋₆ cycloalkyl, 3- to 6-membered heterocyclyl, —(CH₂)ₛCH═CHR¹⁴; the —C₁₋₆ alkyl, aryl, heteroaryl, —C₁₋₆ alkoxy, —C₃₋₆ cycloalkyl and 3- to 6-membered heterocyclyl are optionally substituted or unsubstituted with halogen, OH, NH₂, —C₁₋₆ alkoxy, —C₃, cycloalkyl, substituted C₃₋₆ cycloalkyl, 3- to 6-membered heterocyclyl and substituted 3- to 6-membered heterocyclyl, aryl and heteroaryl.

In some embodiments, R⁵ is a 3- to 6-membered heterocyclyl; the above mentioned 3- to 6-membered heterocyclyl are optionally substituted or not substituted with halogen, OH, NH₂, —C₁₋₆ alkoxy, —C₃₋₆ cycloalkyl, substituted C₃₋₆ cycloalkyl, 3- to 6-membered heterocyclyl, and substituted 3- to 6-membered heterocyclyl, aryl and heteroaryl.

In some embodiments, R⁵ is

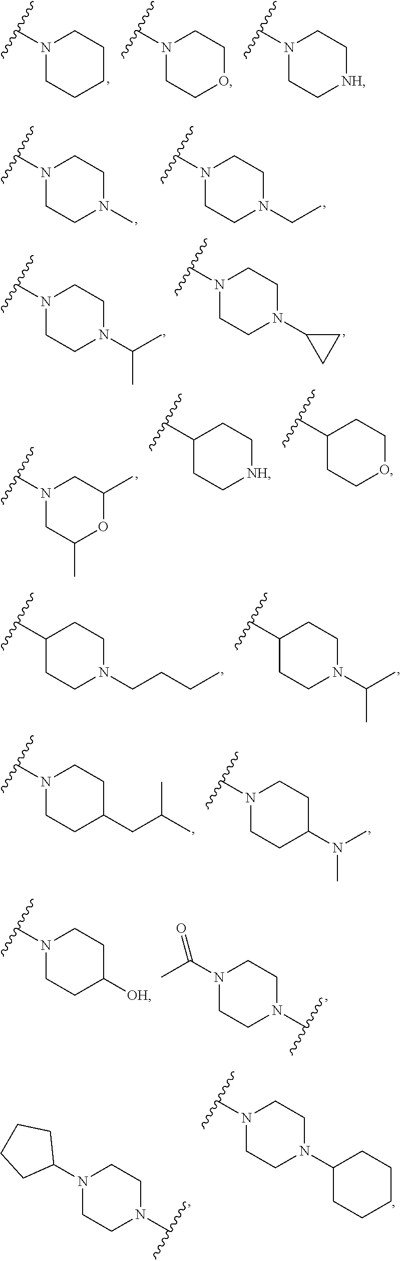

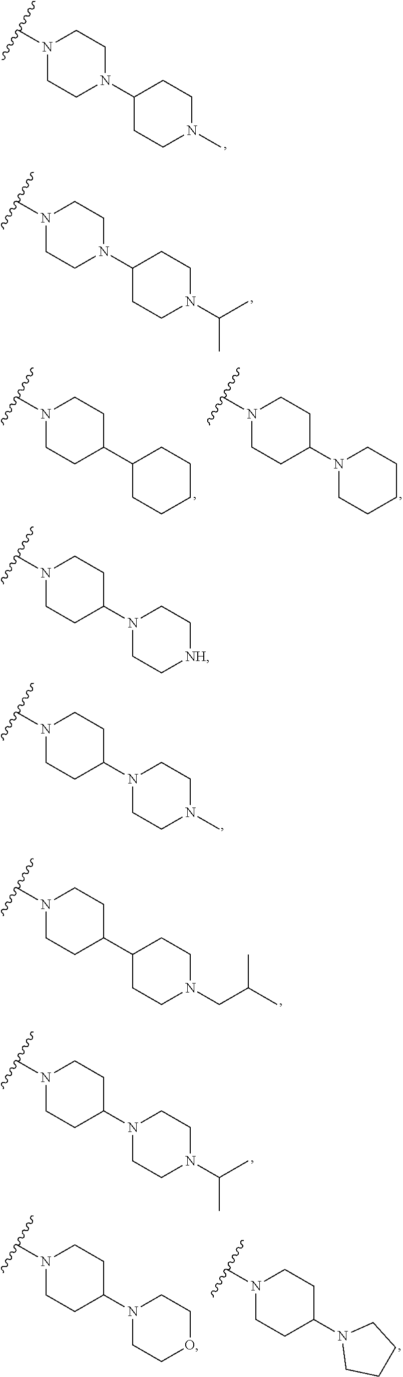

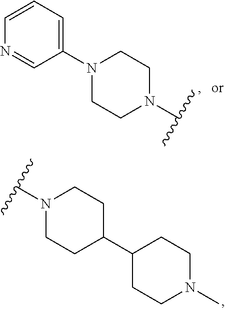 [SMILES: *N1CCN(c2cccnc2)CC1 ; *N1CCC(C2CCN(C)CC2)CC1]

In some embodiments, R⁶ is selected from H, halogen, CN, NO₂, OH, —C₁₋₆ alkyl, aryl, heteroaryl, 3- to 6-membered heterocyclyl, —(CH₂)ₛCH═CHR¹⁴; the above mentioned —C₁₋₆ alkyl, aryl, heteroaryl, and 3- to 6-membered heterocyclyl are optionally substituted or unsubstituted with halogen, OH, NH₂, —C₁₋₆ alkoxy, —C₃₋₆ cycloalkyl, substituted C₃₋₆ cycloalkyl, 3- to 6-membered heterocyclyl and substituted 3- to 6-membered heterocyclyl, aryl and heteroaryl.

In some embodiments, R⁶ is selected from H, methyl, ethyl. CN, allyl,

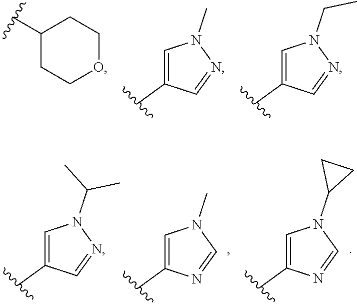

In some embodiments, the compound of Formula I, or its stereoisomer, tautomer, deuterated compound, pharmaceutically acceptable salt, prodrug, chelate, non-covalent complex or solvate are selected from Formula II:

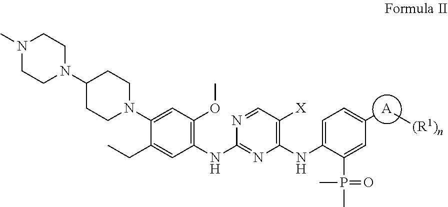

X is halogen;

ring A is a 5- to 10-membered aromatic ring, wherein the 5- to 10-membered aromatic ring is a 5- to 10-membered aryl or a 5- to 10-membered heteroaryl;

R¹ is selected from halogen, CN, OH, —NRR⁸, oxo, —C₁₋₆ alkyl, halogenated C₁₋₆ alkyl, —C₁₋₆ alkoxy, —(CH₂)ₚ(O(CH₂)_{q})ᵣCH₃, —C₁₋₆ cycloalkyl, 3- to 6-membered heterocyclyl, —C₁₋₆ alkyl substituted with 3- to 7-membered heterocyclyl and

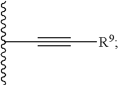

R, R⁸ and R⁹ are independently selected from H, CN, —C₁₋₆ alkyl, —C₃₋₆ cycloalkyl and 3- to 6-membered heterocyclyl, wherein the —C₁₋₆ alkyl, —C₃₋₆ Cycloalkyl and 3- to 6-membered heterocyclyl are optionally substituted or unsubstituted with halogen, OH, NH₂, aryl, heteroaryl, —C₁, alkyl, —C₃₋₆ cycloalkyl, 3- to 6-membered heterocyclyl;

n is any integer selected from 0 to 4;

p is any integer selected from 1 to 3;

s q is any integer selected from 0 to 3;

r is any integer selected from 1 to 3;

In some embodiments, in the Formula II: X is halogen;

R¹ is selected from halogen, cyano, oxo, —N(CH₃)₂, —C₁, alkyl, —C₁, alkyl substituted with 3- to 7-membered heterocyclyl, —C₁₋₆alkoxy or —C₃₋₆ cycloalkyl;

ring A is a 5- to 10-membered aromatic ring;

n is selected from 0, 1 or 2.

In some embodiments, X is fluorine, chlorine, and bromine.

In some embodiments, ring A is a 5- to 10-membered aryl or a 5- to 10-membered heteroaryl; the above mentioned 5- to 10-membered heteroaryl contains 1, 2 or 3 heteroatoms selected from N or S.

In some embodiments, ring A is

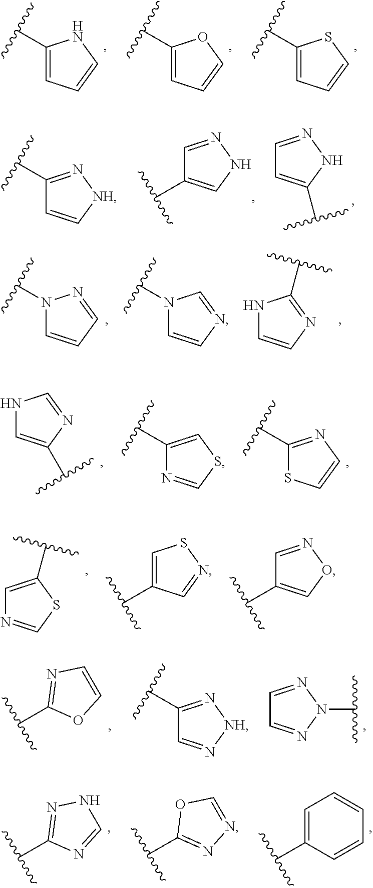

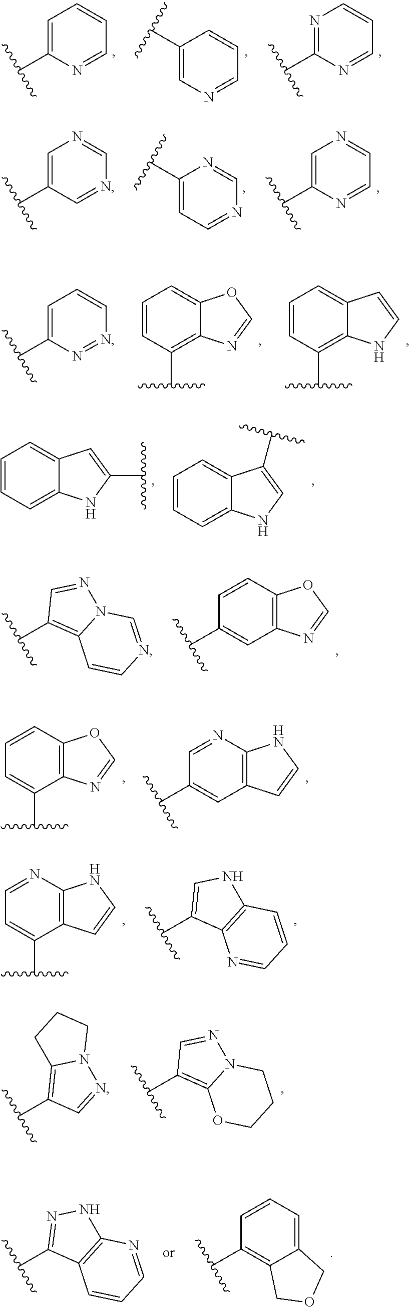

In some embodiments, R¹ is selected from halogen, CN, OH, —NH₂, —N(CH₃)₂, —C₁₋₆ alkyl, haloalkyl, —C₁₋₆ alkoxy, —(CH₂)ₚ(O(CH₂)_{q})ᵣCH₃, —C₃₋₆ cycloalkyl, —C₁₋₆ alkyl substituted with 3- to 7-membered heterocyclyl and

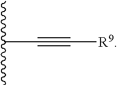

In some embodiments, R¹ is selected from halogen. CN, OH, —NH², —N(CH₃)₂, methyl, methoxy, —CF₃, methoxymethyl, H, oxo, —F, —Cl, —CHF₂,

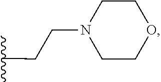

ethoxy, cyclopropyl, ethyl, isopropyl, alkynyl.

In some embodiments, the compound of Formula I is selected from:
(2-((5-Bromo-2-((5-ethyl-2-methoxy-4-(4-(pyrrolidin-1-yl)piperidin-1-yl)phenyl)amino)pyrimidine-4-yl)amino)-5-(1-methyl-1H-pyrazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(4-fluoro-1H-pyrazol-1-yl)phenyl)dimethylphosphine oxide;
(2-((5-Bromo-2-((2-methoxy-4-(4-(4-methylpiperazin-1-yl)piperidin-1-yl)-5-(tetrahydro-2H-pyran-4-yl)phenyl)amino)pyrimidin-4-yl)amino)-5-(thiazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(4-(methoxymethyl)-1H-pyrazol-1-yl)phenyl)dimethylphosphine oxide;
(2-((5-Bromo-2-((5-(1-ethyl-1H-pyrazol-4-yl)-2-methoxy-4-(4-(4-methylpiperazin-1-yl)piperidin-1-yl)phenyl)amino)pyrimidin-4-yl)amino)-5-(1,5-dimethyl-1H-pyrazol-3-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(2-(difluoromethyl)-2H-1,2,3-triazol-4-yl)phenyl)dimethylphosphine oxide;
(6-((5-Bromo-2-((5-ethyl-2-methoxy-4-(4-(4-methylpiperazin-1-yl)piperidin-1-yl)phenyl)amino)pyrimidin-4-yl)amino)-2-fluoro-3-(2-methyl-2H-1,2,3-triazol-4-yl)phenyl)dimethylphosphine oxide;
(2-((5-Bromo-2-((2-methoxy-5-methyl-4-(4-(4-methylpiperazin-1-yl)piperidin-1-yl)phenyl)amino)pyrimidin-4-yl)amino)-5-(2-methyl-2H-1,2,3-triazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(4-ethynyl-1H-pyrazol-1-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-ethynylpyrazin-2-yl)phenyl)dimethylphosphine oxide;
(2-((5-Bromo-2-((2-methoxy-5-methyl-4-(4-(4-methylpiperazin-1-yl)piperidin-1-yl)phenyl)amino)pyrimidin-4-yl)amino)-5-(5-ethynylpyrazin-2-yl)phenyl)dimethylphosphine oxide;
(6-((5-Bromo-2-((5-methoxy-2-methyl-4-(4-(4-methylpiperazin-1-yl)piperidin-1-yl)phenyl)amino)pyrimidin-4-yl)amino)-2-fluoro-3-(2-methyl-2H-1,2,3-triazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(6-ethynylpyrazin-2-yl)phenyl)dimethylphosphine oxide;
(2-((5-Bromo-2-((5-methoxy-2-methyl-4-(4-(4-methylpiperazin-1-yl)piperidin-1-yl)phenyl)amino)pyrimidin-4-yl)amino)-5-(5-ethynylpyrazin-2-yl)phenyl)dimethylphosphine oxide;
(2-((5-Bromo-2-((5-methoxy-2-methyl-4-morpholinophenyl)amino)pyrimidin-4-yl)amino)-5-(2-methyl-2H-1,2,3-triazol-4-yl)phenyl)dimethylphosphine oxide;
(2-((5-Bromo-2-((2-methoxy-5-methyl-4-(4-(4-methylpiperazin-1-yl)piperidin-1-yl)phenyl)amino)pyrimidin-4-yl)amino)-5-(5-ethynylthiophen-2-yl)phenyl)dimethylphosphine oxide;
(2-((5-Bromo-2-((5-methoxy-2-methyl-4-(4-(4-methylpiperazin-1-yl)piperidin-1-yl)phenyl)amino)pyrimidin-4-yl)amino)-5-(pyrazin-2-yl)phenyl)dimethylphosphine oxide;
(2-((5-Bromo-2-((5-methoxy-2-methyl-4-(4-(4-methylpiperazin-1-yl)piperidin-1-yl)phenyl)amino)pyrimidin-4-yl)amino)-5-(4-methoxy-1H-pyrazol-1-yl)phenyl)dimethylphosphine oxide;
(2-((5-Bromo-2-((5-methoxy-2-methyl-4-(4-(4-methylpiperazin-1-yl)piperidin-1-yl)phenyl)amino)pyrimidin-4-yl)amino)-5-(thiazol-2-yl)phenyl)dimethylphosphine oxide;
(2-((2-((4-([1,4′-bipiperidin]-1′-yl)-5-ethyl-2-methoxyphenyl)amino)-5-bromopyrimidine-4-yl)amino)-5-(4-methoxy-1H-pyrazol-1-yl)phenyl)dimethylphosphine oxide;
(2-((5-Bromo-2-((5-methoxy-2-methyl-4-(4-morpholinopiperidin-1-yl)phenyl)amino)pyrimidin-4-yl)amino)-5-(2-methyl-2H-1,2,3-triazol-4-yl)phenyl)dimethylphosphine oxide;
(2-((5-Bromo-2-((5-methoxy-2-methyl-4-morpholinophenyl)amino)pyrimidin-4-yl)amino)-5-(pyrazine-2-yl)phenyl)dimethylphosphine oxide;
(2-((5-Bromo-2-((5-methoxy-2-methyl-4-morpholinophenyl)amino)pyrimidin-4-yl)amino)-5-(4-methoxy-1H-pyrazol-1-yl)phenyl)dimethylphosphine oxide;
(2-((5-Bromo-2-((5-methoxy-2-methyl-4-morpholinophenyl)amino)pyrimidin-4-yl)amino)-5-(thiazol-2-yl)phenyl)dimethylphosphine oxide;
(6-((5-Bromo-2-((5-methoxy-2-methyl-4-morpholinophenyl)amino)pyrimidin-4-yl)amino)-2-fluoro-3-(2-methyl-2H-1,2,3-triazol-4-yl)phenyl)dimethylphosphine oxide;
(2-((5-Bromo-2-((2-cyclopropyl-5-methoxy-4-morpholinophenyl)amino)pyrimidin-4-yl)amino)-5-(4-methoxy-1H-pyrazol-1-yl)phenyl)dimethylphosphine oxide;
5-((5-Bromo-4-((2-(dimethylphosphine oxide)-4-(thiazol-2-yl)phenyl)amino)pyrimidin-2-yl)amino)-4-methoxy-2-morpholinobenzonitrile;
(2-((5-Bromo-2-((5-methoxy-2-methyl-4-(4-methylpiperazin-1-yl)phenyl)amino)pyrimidin-4-yl)amino)-5-(4-methoxy-1H-pyrazol-1-yl)phenyl)-dimethylphosphine oxide;
(2-((5-Bromo-2-((2-methoxy-5-methyl-4-(4-methylpiperazin-1-yl)phenyl)amino)pyrimidin-4-yl)amino)-5-(thiazol-2-yl)phenyl)dimethylphosphine oxide;
(2-((5-Bromo-2-((5-methoxy-2-methyl-4-morpholinophenyl)amino)pyrimidin-4-yl)amino)-5-(1-methyl-1H-pyrazol-4-yl)phenyl)dimethylphosphine oxide;
(2-((5-Bromo-2-((3-methoxy-5-methyl-4-morpholinophenyl)amino)pyrimidin-4-yl)amino)-5-(4-methoxy-1H-pyrazol-1-yl)phenyl)dimethylphosphine oxide;
(2-((5-Bromo-2-((5-methoxy-2-methyl-4-morpholinophenyl)amino)pyrimidin-4-yl)amino)-5-(5-methylpyridine-2-yl)phenyl)dimethylphosphine oxide;
(2-((5-Bromo-2-((5-methoxy-2-methyl-4-morpholinophenyl)amino)pyrimidin-4-yl)amino)-5-(5-cyclopropylpyrazin-2-yl)phenyl)dimethylphosphine oxide;
(6-((5-Bromo-2-((5-ethoxy-2-methyl-4-morpholinophenyl)amino)pyrimidin-4-yl)amino)-2-fluoro-3-(2-methyl-2H-1,2,3-triazol-4-yl)phenyl)dimethylphosphine oxide;
(2-((5-Bromo-2-((5-ethoxy-2-methyl-4-morpholinophenyl)amino)pyrimidin-4-yl)amino)-5-(5-methylpyridine-2-yl)phenyl)dimethylphosphine oxide;
(6-((5-Bromo-2-((2-methyl-4-morpholin-5-(2,2,2-trifluoroethyl)phenyl)amino)pyrimidin-4-yl)amino)-2-fluoro-3-(2-methyl-2H-1,2,3-triazol-4-yl)phenyl)dimethylphosphine oxide;
(6-((5-Bromo-2-((4-(2,6-dimethylmorpholine)-5-methoxy-2-methylphenyl)amino)pyrimidin-4-yl)amino)-2-fluoro-3-(2-methyl-2H-1,2,3-triazol-4-yl)phenyl)dimethylphosphine oxide;
(2-((5-Bromo-2-((4-(4-(dimethylamino)piperidin-1-yl)-5-methoxy-2-methylphenyl)amino)pyrimidine-4-yl)amino)-5-(4-methoxy-1H-pyrazol-1-yl)phenyl)dimethylphosphine oxide;
(2-((5-Bromo-2-((4-(4-hydroxypiperidin-1-yl)-5-methoxy-2-methylphenyl)amino)pyrimidin-4-yl)amino)-5-(4-Methoxy-1H-pyrazol-1-yl)phenyl)dimethylphosphine oxide;
(2-((5-Bromo-2-((5-ethyl-2-methoxy-4-(4-morpholinopiperidin-1-yl)phenyl)amino)pyrimidin-4-yl)amino)-5-(2-methyl-2H-1,2,3-triazol-4-yl)phenyl)dimethylphosphine oxide;
(6-((5-Bromo-2-((5-ethyl-2-methoxy-4-(4-(4-methylpiperazin-1-yl)piperidin-1-yl)phenyl)amino)pyrimidin-4-yl)amino)-2-methoxy-3-(1-methyl-1H-pyrazol-4-yl)phenyl)dimethylphosphine oxide;
(2-((5-Bromo-2-((5-ethyl-2-methoxy-4-(4-morpholinopiperidin-1-yl)phenyl)amino)pyrimidin-4-yl)amino)-5-(1-methyl-1H-pyrazol-4-yl)phenyl)dimethylphosphine oxide;
(2-((5-Bromo-2-((2-methoxy-4-(4-(4-methylpiperazin-1-yl)piperidin-1-yl)-5-propylphenyl)amino)pyrimidin-4-yl)amino)-5-(1-methyl-1H-pyrazol-4-yl)phenyl)dimethylphosphine oxide;
(2-((5-Bromo-2-((4-(4-(dimethylamino)piperidin-1-yl)-5-ethyl-2-methoxyphenyl)amino)pyrimidine-4-yl)amino)-5-(1-methyl-1H-pyrazol-4-yl)phenyl)dimethylphosphine oxide;
(2-((2-((5-Allyl-2-methoxy-4-(4-(4-methylpiperazin-1-yl)piperidin-1-yl)phenyl)amino)-5-bromopyrimidin-4-yl)amino)-5-(1-methyl-1H-pyrazol-4-yl)phenyl)dimethylphosphine oxide;
(6-((5-Bromo-2-((5-ethyl-2-methoxy-4-(4-(4-methylpiperazin-1-yl)piperidin-1-yl)phenyl)amino)pyrimidin-4-yl)amino)-2-methyl-3-(1-methyl-1H-pyrazol-4-yl)phenyl)dimethylphosphine oxide;
(4-((5-Bromo-2-((5-ethyl-2-methoxy-4-(4-morpholinopiperidin-1-yl)phenyl)amino)pyrimidin-4-yl)amino)-[1,1′-biphenyl]-3-yl)dimethylphosphine oxide;
(4-((5-Bromo-2-((5-ethyl-2-methoxy-4-(4-morpholinopiperidin-1-yl)phenyl)amino)pyrimidin-4-yl)amino)-2′-fluoro-[1,1′-biphenyl]-3-yl)dimethylphosphine oxide;
(4-((5-Bromo-2-((5-ethyl-2-methoxy-4-(4-morpholinopiperidin-1-yl)phenyl)amino)pyrimidin-4-yl)amino)-2′-methyl-[1,1′-biphenyl]-3-yl)dimethylphosphine oxide;
(4-((5-Bromo-2-((5-ethyl-2-methoxy-4-(4-(4-methylpiperazin-1-yl)piperidin-1-yl)phenyl)amino)pyrimidin-4-yl)amino)-2-methyl-[1,1′-biphenyl]-3-yl)dimethylphosphine oxide; and
(4-((2-((4-([1,4′-bipiperidin]-1′-yl)-2-methoxy-5-(1-methyl-1H-pyrazole-4-yl)phenyl)amino)-5-bromopyrimidin-4-yl)amino)-2-methyl-[1,1′-biphenyl]-3-yl)dimethylphosphine oxide;
(5-(Benzo[d]oxazol-4-yl)-2-((5-chloro-2-((5-ethyl-2-methoxy-4-(4-(4-methylpiperazin-1-yl)piperidin-1-yl)phenyl)amino)pyrimidin-4-yl)amino)phenyl)dimethylphosphine oxide;
(5-(2-Aminothiazol-4-yl)-2-((5-chloro-2-((5-ethyl-2-methoxy-4-(4-(4-methylpiperazin-1-yl)piperidin-1-yl)phenyl)amino)pyrimidin-4-yl)amino)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(4-methoxy-1H-pyrazol-1-yl)phenyl)dimethylphosphine oxide;
(5-(3-Aminopyridin-2-yl)-2-((5-chloro-2-((5-ethyl-2-methoxy-4-(4-(4-methylpiperazin-1-yl)piperidin-1-yl)phenyl)amino)pyrimidin-4-yl)amino)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-isopropylpyrimidin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(2-(dimethylamino)pyrimidin-5-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-ethoxypyrimidin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(2-ethoxypyrimidin-5-yl)phenyl)dimethylphosphine oxide;
3-(4-((5-Chloro-2-((5-ethyl-2-methoxy-4-(4-(4-methylpiperazin-1-yl)piperidin-1-yl)phenyl)amino)pyrimidin-4-yl)amino)-3-(dimethylphosphine oxide)phenyl)-1-methyl-1H-pyrazol-4-carbonitrile;
(2-((5-Chloro-2-((5-ethyl-2-methoxy-4-(4-(4-methylpiperazin-1-yl)piperidin-1-yl)phenyl)amino)pyrimidin-4-yl)amino)-5-(3-ethoxy-5-methylisoxazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(2-ethyl-2H-1,2,3-triazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(pyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-cyclopropylpyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-methylpyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(1,5-dimethyl-1H-pyrazol-3-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5,6-dimethylpyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(2-isopropyl-2H-1,2,3-triazol-4-yl)phenyl)di methylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(4-methyl-1H-pyrazol-1-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(6-methylpyridin-3-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-methylpyridin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-methoxythiophen-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(4-chlorothiazol-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-methoxypyridin-2-yl)phenyl)dimethylphosphine oxide;
(6-((5-Bromo-2-((5-methoxy-2-methyl-4-(4-(4-methylpiperazin-1-yl)piperidin-1-yl)phenyl)amino)pyrimidin-4-yl)amino)-2-fluoro-3-(4-methoxy-1H-pyrazol-1-yl)phenyl)dimethylphosphine oxide;
(2-((5-Bromo-2-((2-methoxy-5-methyl-4-(4-(4-methylpiperazin-1-yl)piperidin-1-yl)phenyl)amino)pyrimidin-4-yl)amino)-5-(4-methoxy-1H-pyrazol-1-yl)phenyl)dimethylphosphine oxide;
(6-((5-Bromo-2-((2-methoxy-5-methyl-4-(4-(4-methylpiperazin-1-yl)piperidin-1-yl)phenyl)amino)pyrimidin-4-yl)amino)-2-fluoro-3-(4-fluoro-1H-pyrazol-1-yl)phenyl)dimethylphosphine oxide;
(4-((5-Bromo-2-((5-ethyl-2-methoxy-4-(morpholinopiperidin-1-yl)phenyl)amino)pyrimidin-4-yl)amino)-2′-methyl-[1,1′-diphenyl]-3-yl)dimethylphosphine oxide;
(2-((5-Bromo-2-((4-(4-(dimethylamino)piperidin-1-yl)-5-ethyl-2-methoxyphenyl)amino)pyrimidine-4-yl)amino)-5-(1-methyl-1H-pyrazolo[3,4-b]pyridin-3-yl)phenyl)dimethylphosphine oxide;
(2-((5-Bromo-2-((5-(1-ethyl-1H-pyrazol-4-yl)-2-methoxy-4-(4-(4-methylpiperazin-1-yl)piperidin-1-yl)phenyl)amino)pyrimidin-4-yl)amino)-5-(1,5-dimethyl-1H-pyrazol-3-yl)phenyl)dimethylphosphine oxide;
(2-((5-Bromo-2-((5-((4-(4-cyclopentylpiperazin-1-yl)-2-methoxy-5-(1-methyl-1H-Pyrazol-4-yl)phenyl)amino)pyrimidin)-4-yl)amino)-5-(4-methoxy-1H-pyrazol-1-yl)phenyl)dimethylphosphine oxide;
1-(4-(4-((5-Bromo-4-((2-(dimethylphosphate)-4-(4-methoxy-1H-pyrazol-1-yl)phenyl)amino)pyrimidin)-2-yl)amino)-5-methoxy-2-(1-methyl-1H-pyrazol-4-yl)phenyl)piperazin-1-yl-acetylacetone;
(2-((5-Bromo-2-((2-methoxy-5-(1-methyl-1H-pyrazol-4-yl)-4-(4-(pyridin-3-yl)piperazin-1-yl)phenyl)amino)pyrimidin-4-yl)amino)-5-(4-methoxy-1H-imidazol-1-yl)phenyl)dimethylphosphine oxide;
(2-((5-Bromo-2-((5-methoxy-2-methyl-4-(4-(4-methylpiperazin-1-yl)piperidin-1-yl)phenyl]amino]amino]pyrimidin-4-yl)amino)-5-(4-(trifluoromethyl)-1H-pyrazol-1-yl)phenyl)dimethylphosphine oxide;
(2-((5-Bromo-2-((2-methoxy-5-(1-methyl-1H-pyrazol-4-yl)-4-(4-(4-methylpiperazin-1-yl)piperidin-1-yl)phenyl)amino)pyrimidin-4-yl)amino)-5-(4-(trifluoromethyl)-1H-pyrazol-1-yl)phenyl)dimethylphosphine oxide;
(2-((5-Bromo-2-((2-methoxy-5-methyl-4-(4-(4-methylpiperazin-1-yl)piperidin-1-yl)phenyl)amino]amino]pyrimidin-4-yl)amino)-5-(4-(trifluoromethyl)-. H-pyrazol-1-yl)phenyl)dimethylphosphine oxide;
(2-((2-((4-([[1,4′-bipiperidin]-1-1-yl)-2-methoxy-5-methylphenyl)amino)-5-bromopyrimidin-4-yl)amino)-5-(4-methoxy-1H-pyrazol-1-yl)phenyl)dimethylphosphine oxide;
(2-((5-Bromo-2-((2-methoxy-5-methyl-4-(4-(1-methylpiperidin-4-yl)piperazin-1-yl)phenyl)amino]amino]pyrimidin-4-yl)amino)-5-(4-methoxy-1H-pyrazol-1-yl)phenyl)dimethylphosphine oxide;
(2-((5-Bromo-2-((4-(4-cyclopentylpiperazin-1-yl)-2-methoxy-5-methylphenyl)amino)pyrimidin-4-yl)amino)-5-(4-methoxy-1H-pyrazol-1-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(4-(methoxymethyl)-1H-pyrazol-1-yl)phenyl)dimethylphosphine oxide;
(2-((5-Bromo-2-((5-(1-ethyl-1H-pyrazol-4-yl)-2-methoxy-4-(4-(4-methylpiperazin-1-yl)piperidin-1-yl)phenyl)amino)pyrimidin-4-yl)amino)-5-(1H-indol-7-yl)phenyl)dimethylphosphine oxide;
3-(4-((5-Chloro-2-((5-ethyl-2-methoxy-4-(4-(4-methylpiperazin-1-yl)piperidin-1-yl)phenyl)amino)pyrimidin-4-yl)amino)-3-(dimethylphosphoryl)phenyl)pyridin-2(1H)-quinoxalinone;
(4-((5-Bromo-2-((5-ethyl-2-methoxy-4-(4-morpholinopiperidin-1-yl)phenyl)amino)pyrimidin-4-yl)amino)-2′-fluoro-[1,1′-biphenyl]-3-yl)dimethylphosphine oxide;
(2-((5-Bromo-2-((5-ethyl-2-methoxy-4-(4-morpholinopiperidin-1-yl)phenyl)amino)pyrimidin-4-yl)amino)-5-(2-methyl-2H-1,2,3-triazol-4-yl)phenyl)dimethylphosphine oxide;
(5-(2-Aminothiazol-4-yl)-2-((5-chloro-2-((5-ethyl-2-methoxy-4-(4-(4-methylpiperazin-1-yl)piperidin-1-yl)phenyl)amino)pyrimidin-4-yl)amino)phenyl)dimethylphosphine oxide;
(2-((5-Bromo-2-((2-methoxy-4-(4-(4-methylpiperazin-1-yl)piperidin-1-yl)-5-(tetrahydro-2H-pyran-4-yl)phenyl)amino)pyrimidin-4-yl)amino)-5-(thiazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(6-hydroxypyridazin-3-yl)phenyl)dimethylphosphine oxide;
(5-(3-Aminopyridin-2-yl)-2-((5-bromo-2-((5-ethyl-2-methoxy-4-(4-(4-methylpiperazin-1-yl)piperidin-1-yl)phenyl)amino)pyrimidin-4-yl)amino)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(4-hydroxypyrimidin-5-yl)phenyl)dimethylphosphine oxide;
(4-((2-((4-(4-([1,4′-bipiperidin]-1′-yl)-2-methoxy-5-(1-methyl-1H-pyrazole)-4-yl)phenyl)amino)-5-bromopyrimidin-4-yl)amino)-[1,1′-biphenyl]-3-yl)dimethylphosphine oxide;
(2-((5-Bromo-2-((5-ethyl-2-methoxy-4-(4-(4-methylpiperazin-1-yl)piperidin-1-yl)phenyl)aminopyrimidin-4-yl)amino)-5-(1,5-dimethyl-1H-pyrazol-3-yl)phenyl dimethylphosphine oxide;
(2-((5-Bromo-2-((5-(1-ethyl-1H-pyrazol-4-yl)-2-methoxy-4-(4-(4-methylpiperazin-1-yl)piperidin-1-yl)phenyl)amino)pyrimidin-4-yl)amino)-5-(1-methyl-1H-pyrazolo[3,4-b]pyridin-3-yl)phenyl)dimethylphosphine oxide;
(5-(3-Aminopyridin-2-yl)-2-((5-chloro-2-((5-ethyl-2-methoxy-4-(4-(4-methylpiperazin-1-yl)piperidin-1-yl)phenyl)amino)pyrimidin-4-yl)amino)phenyl)dimethylphosphine oxide;
2-((5-Chloro-2-((5-ethyl-2-methoxy-4-(4-(4-methylpiperazin-1-yl)piperidin-1-yl)phenyl)amino)pyrimidin-4-yl)amino)-5-(4,5,6,7-tetrahydropyrazolo[1,5-a]pyridin-3-yl)phenyl)dimethylphosphine oxide;
(4-((5-Bromo-2-((5-ethyl-2-methoxy-4-(4-morpholinopiperidin-1-yl)phenyl)amino)pyrimidin-4-yl)amino)-[1,1′-biphenyl]-3-yl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(2-(difluoromethyl)-2H-1,2,3-triazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(1H-pyrazol-5-yl)phenyl)dimethylphosphine oxide;
(2-((5-Chloro-2-((5-ethyl-2-methoxy-4-(4-(4-methylpiperazin-1-yl)piperidin-1-yl)phenyl)amino)pyrimidin-4-yl)amino)-5-(1-methyl-1-imidazol-2-yl)phenyl)dimethylphosphine oxide;
(2-((5-Chloro-2-((5-ethyl-2-methoxy-4-(4-(4-methylpiperazin-1-yl)piperidin-1-yl)phenyl)amino)pyrimidin-4-yl)amino)-5-(5,5-dimethyl-5,6-dihydro-tetrahydro-pyrrole[1,2-b]pyrazol-3-)phenyl)dimethylphosphine oxide;
(2-((5-Bromo-2-((5-ethyl-2-methoxy-4-(-4-(4-methylpiperazin-1-yl)piperidin-1-yl)phenyl)amino)pyrimidin-4-yl)amino)-5-(1-methyl-1H-pyrrolo[3,2-b]pyridin-3-yl)phenyl)dimethylphosphine oxide;
2-(4-((5-Bromo-2-((5-ethyl-2-methoxy-4-(4-(4-methylpiperazin-1-yl)piperidin-1-yl)Phenyl)amino)pyrimidin-4-yl)amino)-3-(dimethylphosphino)phenyl)-1H-indole-5-carbonitrile;
(2-((5-Bromo-2-((5-ethyl-2-methoxy-4-(4-(4-methylpiperazin-1-yl)piperidin-1-yl)phenyl)amino)pyrimidin-4-yl)amino)-5-(4-fluoro-1H-pyrazol-1-yl)phenyl)dimethylphosphine oxide;
(2-((5-Chloro-2-((5-ethyl-2-methoxy-4-(4-(4-methylpiperazin-1-yl)piperidin-1-yl)phenyl)amino)pyrimidin-4-yl)amino)-5-(5-methyl-1,3,4-oxadiazol-2-yl)phenyl)dimethylphosphine oxide;
(2-((5-Bromo-2-((5-ethyl-2-methoxy-4-(-4-(4-methylpiperazin-1-yl)piperidin-1-yl)phenyl)amino)pyrimidin-4-yl)amino)-5-(1-methyl-1H-pyrrolo[2,3-b]pyridin-3-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(1-(2-morpholinoethyl)-1H-pyrazol-4-yl)phenyl)dimethylphosphine oxide;
(2-((5-Chloro-2-((5-ethyl-2-methoxy-4-(4-(4-methylpiperazin-1-yl)piperidin-1-yl)phenyl)amino)pyrimidin-4-yl)amino)-5-(1H-pyrazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(1H-pyrazol-1-yl)phenyl)dimethylphosphine oxide;
(2-((5-Bromo-2-((5-ethyl-2-methoxy-4-(4-(4-methylpiperazin-1-yl)piperidin-1-yl)phenyl)amino]amino]pyrimidin-4-yl)amino)-5-(4-methoxy-1H-pyrazol-1-yl)phenyl)dimethylphosphine oxide;
4′-((5-Bromo-2-((5-ethyl-2-methoxy-4-(4-(4-methylpiperazin-1-yl)piperidin-1-yl)phenyl)amino)pyrimidin-4-yl)amino)-3′-(dimethylphosphoryl)-[1,1′-biphenyl]-2-carbonitrile;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(1H-indol-7-yl)phenyl)dimethylphosphine oxide;
(2-((5-Chloro-2-((5-ethyl-2-methoxy-4-(4-(4-methylpiperazin-1-yl)piperidin-1-yl)phenyl)amino)pyrimidin-4-yl)amino)-5-(4-methoxypyridin-3-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(1,4-dimethyl-1H-pyrazol-5-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(1-methyl-1H-pyrrolo[2,3-b]pyridin-5-yl)phenyl)dimethylphosphine oxide;
(2-((5-Chloro-2-((5-ethyl-2-methoxy-4-(4-(4-methylpiperazin-1-yl)piperidin-1-yl)phenyl)amino)pyrimidin-4-yl)amino)-5-(1-methyl-1H-pyrrolo[2,3-b]pyridin-4-yl)phenyl)dimethylphosphine oxide;
3-(4-((5-Chloro-2-((5-ethyl-2-methoxy-4-(4-(4-methylpiperazin-1-yl)piperidin-1-yl)phenyl)amino)pyrimidin-4-yl)amino)-3-(dimethylphosphoryl)phenyl)-1-methyl-1H-pyrazol-4-carbonitrile;
(2-((5-Chloro-2-((5-ethyl-2-methoxy-4-(4-(4-methylpiperazin-1-yl)piperidin-1-yl)phenyl)amino)pyrimidin-4-yl)amino)-5-(3-ethoxy-5-methylisoxazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(6-methoxypyrazin-2-yl)phenyl)dimethylphosphine oxide; (2-((5-Bromo-2-((5-ethyl-2-methoxy-4-(4-(4-methylpiperazin-1-yl)piperidin-1-yl)phenyl)amino)amino]pyrimidin-4-yl)amino)-5-(5-methylthiazol-4-yl)phenyl)dimethylphosphine oxide;
(5-(Benzo[d]oxazol-4-yl)-2-((5-chloro-2-((5-ethyl-2-methoxy-4-(4-(4-methylpiperazin-1-yl)piperidin-1-yl)phenyl)amino)pyrimidin-4-yl)amino)phenyl)dimethylphosphine oxide;
(2-((5-Bromo-2-((5-ethyl-2-methoxy-4-(4-(4-methylpiperazin-1-yl)piperidin-1-yl)phenyl)amino]pyrimidin-4-yl)amino)-5-(1-methyl-1H-1,2,4-triazol-5-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(3-methoxy-1-methyl-1H-pyrazol-4-yl)phenyl)dimethylphosphine oxide;
4-(4-((5-Bromo-2-((5-ethyl-2-methoxy-4-(4-(4-methylpiperazin-1-yl)piperidin-1-yl)phenyl)amino]pyrimidin-4-yl)amino)-3-(dimethylphosphoryl)phenyl)-1-methyl-1H-pyrazol-3-carbonitrile;
(2-((5-Chloro-2-((5-ethyl-2-methoxy-4-(4-(4-methylpiperazin-1-yl)piperidin-1-yl)phenyl)amino)pyrimidin-4-yl)amino)-5-(4-methoxypyrimidin-5-yl)phenyl)dimethylphosphine oxide;
(2-((5-Chloro-2-((5-ethyl-2-methoxy-4-(4-(4-methylpiperazin-1-yl)piperidin-1-yl)phenyl)amino)pyrimidin-4-yl)amino)-5-(4-methylpyrimidin-5-yl)phenyl)dimethylphosphine oxide;
(2-((5-Chloro-2-((5-ethyl-2-methoxy-4-(4-(4-methylpiperazin-1-yl)piperidin-1-yl)phenyl)amino)pyrimidin-4-yl)amino)-5-(2-methylpyrimidin-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(1-methyl-1H-imidazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(1-cyclopropyl-1H-imidazol-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-ethylpyrimidin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(6-(dimethylamino)pyridin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(3-methoxypyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-isopropylpyrimidin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(2-(dimethylamino)pyrimidin-5-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(5-ethoxypyrimidin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(2-ethoxypyrimidin-5-yl)phenyl)dimethylphosphine oxide;
(2-((5-Bromo-2-((5-ethyl-2-methoxy-4-(4-(4-methylpiperazin-1-yl)piperidin-1-yl)phenyl)amino]pyrimidin-4-yl)amino)-5-(6-(dimethylamino)pyridazin-3-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(pyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-cyclopropylpyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-methylpyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5,6-dimethylpyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(6-methylpyridin-3-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-methylpyridin-2-yl)phenyl)dimethylphosphine oxide;
(6-((5-Bromo-2-((5-ethyl-2-methoxy-4-(4-(4-methylpiperazin-1-yl)piperidin-1-yl)phenyl)amino)pyrimidin-4-yl)amino)-2-fluoro-3-(2-methyl-2H-1,2,3-triazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(2-isopropyl-2H-1,2,3-triazol-4-yl)phenyl)dimethylphosphine oxide; and
(2-((5-Bromo-2-((5-ethyl-2-methoxy-4-(4-(4-methylpiperazin-1-yl)piperidin-1-yl)phenyl)amino)pyrimidin-4-yl)amino)-5-(4-methyl-1H-pyrazol-1-yl)phenyl)dimethylphosphine oxide.

The second purpose of the present invention provides a pharmaceutical composition includes the compound of Formula I, or its stereoisomer, tautomer, deuterated compound, pharmaceutically acceptable salt, prodrug, chelate, non-covalent complex or solvate as the active component, and at least one pharmaceutically acceptable adjuvant, such as carrier or excipient.

The third purpose of the present invention further provides methods for inhibiting various forms of EGFR, including one or various combination of L858R, Δ19del, T790M and C797S, the method comprising administering to a patient any one of the compounds shown by Formula I, or stereoisomer, tautomer, deuterated compound, pharmaceutically acceptable salt, prodrug, chelate, non-covalent complex or its solvate thereof.

The fourth purpose of the present invention also provides a method for treating EGFR-driven cancer, the method comprising administering to a patient in need thereof a therapeutically effective amount of any of the compounds shown by Formula I, or a stereoisomer, tautomer, deuterated compound, pharmaceutically acceptable salt, prodrug, chelate, non-covalent complex or solvate thereof.

(i) C797S, (ii) L858R and C797S, (iii) C797S and T790M. (iv) L858R. T790M, and C797S, or (v) Δ19del, T790M and C797S. In some embodiments, the EGFR-driven cancer is characterized by the presence of one or more mutations from: (i) C797S. (ii) L858R and C797S. (iii) C797S and T790M, (iv) L858R. T790M and C797S, or (v) Δ19del, T790M and C797S.

In some implementations, the EGFR-driven cancers involve colon cancer, stomach cancer, thyroid cancer, lung cancer, leukemia, pancreatic cancer, melanoma, brain cancer, kidney cancer, prostate cancer, ovarian cancer, or breast cancer.

In some implementations, the mentioned lung cancer is non-small cell lung cancer caused by EGFR^{L858R/T790M/C797S} or EGFR^{Δ19del/T790M/C797S} mutant.

The fifth purpose of the present invention provides a method for inhibiting mutant EGFR in a patient, the method comprising administering to a patient in need thereof a therapeutically effective amount of any of the compounds shown by Formula I, or a stereoisomer, tautomer, deuterated compound, pharmaceutically acceptable salt, prodrug, chelate, non-covalent complex or solvate thereof.

The sixth purpose of the present invention provides use of any of the compounds shown by Formula I, or a stereoisomer, tautomer, deuterated compound, pharmaceutically acceptable salt, prodrug, chelate, non-covalent complex, solvate or pharmaceutical composition thereof in preparation of medicines.

In some implementations, wherein the mentioned drugs are used to treat or prevent cancer.

In some implementations, wherein the cancers involve colon cancer, stomach cancer, thyroid cancer, lung cancer, leukemia, pancreatic cancer, melanoma, brain cancer, kidney cancer, prostate cancer, ovarian cancer, or breast cancer.

In some implementations, the mentioned lung cancer is non-small cell lung cancer caused by EGFRL^{858R/T790M/C797S} or EGFR^{Δ19del/T790M/C797S} mutant.

### Definition and Description

The general chemical terms used in the formula above have their usual meanings. For example, the term “halogen”, as used herein, unless otherwise indicated, means fluoro, chloro, bromo or iodo. The preferred halogen groups include F, Cl and Br.

Unless otherwise indicated, alkyl includes saturated monovalent hydrocarbon radicals having straight or branched chains. For example, alkyl includes methyl, ethyl, propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl and 2-methylpentyl. Similarly, C₁₋₈, as in C₁₋₈ alkyl is defined to identify the group as having 1, 2, 3, 4, 5, 6, 7 or 8 carbon atoms in a straight or branched arrangement.

Similarly, C₃₋₆, as in C₃₋₆ cycloalkyl is defined to identify the group as having 3, 4, 5, 6, 7 or 8 carbon atoms in a substituted or unsubstituted circular arrangement.

Alkoxy radicals are oxygen ethers formed from the previously described straight, branched chain or cyclic alkyl groups.

Unless otherwise indicated, the term “aromatic ring” refers to mono- or polycyclic aromatic system, at least one ring is aromatic. The “aromatic ring” may or may not contain heteroatoms, which are nitrogen, oxygen and sulfur, and the sulfur as used herein can be further oxo-substituted. It is known that when the “aromatic ring” substituent is a bicyclic substituent and one of the rings is a non-aromatic, the attachment to the benzene ring in the general formula is carried out through the aromatic system.

The term “aryl”, as used herein, unless otherwise indicated, refers to an unsubstituted or substituted mono- or polycyclic ring system containing carbon ring atoms, and at least one ring is aromatic. The preferred aryls are mono cyclic or bicyclic 6-10 membered aromatic ring systems. Phenyl and naphthyl are preferred aryls. The most preferred aryl is phenyl.

Unless otherwise indicated, the term “heteroaryl”, as used herein, represents an unsubstituted or substituted stable 5- or 6-membered monocyclic aromatic ring system or unsubstituted or substituted 9- or 10-membered benzo-fused heteroaromatic ring system or bicyclic heteroaromatic ring system, and at least one ring is aromatic. It consists of carbon atoms and one to four heteroatoms selected from N, O or S, and wherein the nitrogen or sulfur heteroatoms may optionally be oxidized, and the nitrogen heteroatom may optionally be quaternized. The heteroaryl group may be attached at any heteroatom or carbon atom which results in the creation of a stable structure. Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyl adeninyl, quinolinyl or isoquinolinyl.

The term “cycloalkyl” refers to a cyclic saturated alkyl chain with carbon atoms, such as cyclopropyl, cyclobutyl, cyclopentyl, or cyclohexyl.

Unless otherwise indicated, the term “heterocyclyl”, as used herein, represents saturated or partially unsaturated stable mono-, bi- and tricyclic structure containing heteroatom groups. They consist of carbon atoms one to four heteroatoms selected from N, O or S, and wherein the nitrogen or sulfur heteroatoms may optionally be oxidized, and the nitrogen heteroatom may optionally be quaternized. The heteroaryl group may be attached at any heteroatom or carbon atom which results in the creation of a stable structure. Examples of heteroaryl groups include, but are not limited to oxiranyl, piperazinyl, morpholinyl, piperidinyl, tetrahydropyrrolyl, tetrahydrofuryl, etc.

Unless otherwise indicated, the term “haloalkyl”, as used herein, refers to straight- or branched-chain alkyl containing 1-6 halogen. The term “halogen” used herein refers to F, Cl, Br or I. For example, haloalkyl includes monofluoromethyl, difluoromethyl, trifluoromethyl, Trifluoroethyl, etc.

The term “substituted” refers to a group in which one or more hydrogen atoms are each independently substituted with the same or different substituents. Typical substituents include but are not limited to halogen (F, Cl, Br or I), C₁₋₈ alkyl, C₃₋₁₂ cycloalkyl, —OR¹, SR¹, ═O, ═S, —C(O)R¹, —C(S)R¹, —NR¹, —C(O)OR¹, —C(S)OR¹, —NR¹R², —C(O)NR¹R², cyano, nitro, —S(O)₂R¹, —OS(O₂)OR¹, —OS(O)₂R¹, —OP(O)(OR¹)OR²), wherein R¹ and R² are independently selected from —H, lower alkyl, and lower halogenated alkyl. In some embodiments, the substituents are independently selected from —F, —Cl, —Br, —I, —OH, trifluoromethoxy, ethoxy, propoxy, isopropoxy, n-butoxy, Isobutoxy, t-butoxy, —SCH₃, —SC₂H₅, formyl, —C(OCH₃), cyano, nitro, CF₃, —OCF₃, amino, dimethylamino, methylthio, sulfonyl and acetyl.

Embodiments of substituted alkyl include, but are not limited to, 2-aminoethyl, 2-hydroxyethyl, pentachloroethyl, trifluoromethyl, methoxymethyl, pentafluoroethyl, and piperazinylmethyl.

Embodiments of substituted alkoxy include, but are not limited to, aminomethoxy, tetrafluoromethoxy, 2-diethylaminoethoxy, 2-ethoxycarbonylethoxy, 3-hydroxypropoxy.

In some embodiments, when the ring A, as used herein, is

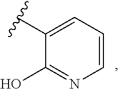

it can be transformed to

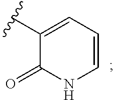

when N in the amide bond of

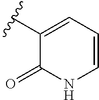

structure is further replaced, it is also intended to be encompassed within the scope of this invention; similarly,

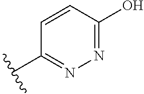

can be transformed into

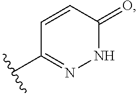

and so on.

The term “composition”, as used herein, is intended to encompass a product comprising the specific ingredients in the specific amounts, as well as any product which results, directly or indirectly, from combinations of the specific ingredients in the specific amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

The compounds of the present invention may also present in the form of pharmaceutically acceptable salts. For use in medicine, the salts of the compounds in this invention refer to a non-toxic “pharmaceutically acceptable salts”. The forms of pharmaceutically acceptable salts include pharmaceutically acceptable acidic/anionic or basic/cationic salts. The pharmaceutically acceptable acid/anionic salts usually take the form of protonation of inorganic or organic acid for basic nitrogen. Representative organic or inorganic acids include hydrochloric acid, hydrobromic acid, hydroiodic acid, perchloric acid, sulfuric acid, nitric acid, phosphoric acid, acetic acid, propionic acid, glycolic acid, lactic acid, succinic acid, maleic acid, fumaric acid, apple Acid, tartaric acid, citric acid, benzoic acid, mandelic acid, methanesulfonic acid, oxyethylsulfonic acid, benzenesulfonic acid, oxalic acid, pamoic acid, 2-naphthalenesulfonic acid, p-toluenesulfonic acid, cyclohexane sulfamic acid, Salicylic acid, saccharin or trifluoroacetic acid. Pharmaceutically acceptable alkaline/cationic salts include, but are not limited to, aluminum, calcium, chloroprocaine, choline, diethanolamine, ethylenediamine, lithium, magnesium, potassium, sodium, and zinc.

The present invention includes within its scope the prodrugs of the compounds of this invention. Generally, the prodrugs refer to functional derivatives that are easily converted in vivo into desired compounds. Therefore, in the therapeutic method of the present invention, the term “administration” shall include the treatment of various conditions described for specific disclosed compounds, or the use of compounds that may not be specifically disclosed, but are converted into specific compounds in vivo after administration. Conventional methods for selecting and preparing suitable derivatives of prodrugs have been documented in books Design of Prodrugs (ed. H. Bundgaard, Elsevier, 1985), etc.

Obviously, the definition of any substituent or variable at a particular location in a molecule is independent of any other position in the molecule. It is easy to understand that the substituent or substituted patterns of the compound of the present invention can be selected by ordinary technicians to provide compounds that are chemically stable and that can be readily synthesized by techniques know in the art as well as those methods set forth herein.

The present invention includes compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof.

The above Formula I are shown without a definitive stereochemistry at certain positions. The present invention includes all stereoisomers of the compounds shown by Formula I and their pharmaceutically acceptable salts. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound shown by Formula I exists, the present invention includes any possible tautomers and pharmaceutically acceptable salts thereof, and mixtures thereof, except where specifically stated otherwise.

When the compound of Formula I and pharmaceutically acceptable salts thereof exist in the form of solvates or polymorphic forms, the present invention includes any possible solvates and polymorph. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline. N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, formic, hydrochloric, maleic, phosphoric, sulfuric and tartaric acids, particularly preferred are formic and hydrochloric acid. Since the compounds of Formula I are intended for pharmaceutical use they are preferably provided in substantially pure form, for example at least 60% pure, more suitably at least 75% pure, especially at least 98% pure (% are on a weight for weight basis).

The pharmaceutical compositions of the present invention comprise a compound represented by Formula I (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I, or a prodrug, or a metabolite, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula I, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently prepared into the desired appearance.

Thus, the pharmaceutical compositions of this invention may include a pharmaceutically acceptable carrier compounds shown by Formula I or their tautomers, polymorphs, solvates, pharmaceutically acceptable salts and prodrugs. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include such as lactose, terra alba, sucrose, talc, gelatin, agar, pectin, acacia, magnesium stearate, and stearic acid. Examples of liquid carriers include such as sugar syrup, peanut oil, olive oil, and water. Examples of gaseous carriers include such as carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, so elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their case of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg or 1000 mg.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier (s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

Generally, dosage levels on the order of from about 0.01 mg/kg to about 150 mg/kg of body weight per day are useful in the treatment of the above-indicated conditions, or alternatively about 0.5 mg to about 7 g per patient per day. For example, inflammation, cancer, psoriasis, allergy/asthma, diseases and discomforts of the immune system, and diseases and discomforts of the central nervous system (CNS), are effectively treated at the dosage levels of the drug ranging from about 0.01 to 50 mg of the compound per kilogram of body weight per day, or alternatively about 0.5 mg to about 3.5 g per patient per day.

It is understood, however, that lower or higher doses than those recited above may be required. Specific dose level and treatment regimens for any particular subject will depend upon a variety of factors, including the activity of the specific compound employed, the age, body weight, general health, sex, diet, time of administration, route of administration, rate of excretion, drug combination, the severity and course of the particular disease undergoing therapy.

These and other aspects will become apparent from the following written description of the invention.

The following Examples are provided to better illustrate the present invention. All parts and percentages are by weight and all temperatures are degrees Celsius, unless explicitly stated otherwise.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. According to at least one of the assay methods described herein, the compounds of the embodiments have been found to have the ability to inhibit L858R, Δ19del, T790M and C797S.

### EXAMPLES

It should be understood that the preceding general descriptions and the detailed descriptions below are exemplary and illustrative only and are not a restriction on any subject requiring protection. Unless expressly stated otherwise, all portions and percentages are by weight, and all temperature units are in degrees Celsius. The compounds described herein can be obtained from commercial sources or synthesized by conventional methods as shown below using commercially available raw materials and reagents.

The following abbreviations have been used in embodiments:

DIEA: N,N-diisopropylethylamine;

DMF: N,N-dimethylformamide;

DMSO: Dimethyl sulfoxide;

HEPES: 4-(2-hydroxyethyl)-1-piperazine ethanesulfonic acid;

LCMS: Liquid Chromatography-Mass Spectrometry;

h or hrs: hours;

Pd/C: Palladium on active carbon;

MeOH: Methanol;

TLC: Thin Layer Chromatography;

Pd(OAc)₂: Palladium acetate;

n-BuOH: n-butanol;

TsOH: p-toluenesulfonic acid;

PTLC: Prepare Thin Layer Chromatography;

Xantphos: 4,5-bis-diphenylphosphanyl-9,9-dimethyl-9h-xanthene;

PCy₃: Tricyclohexylphosphine;

Pd₂(dba)₃: Tridibenzyl acetone dipalladium

t-BuXPhos: 2-Di-tert-butylphosphino-2′,4′,6′-triisopropylbiphenyl

KF: Potassium fluoride.

### Example 1: Synthesis of Compound K-018

### (2-((5-Bromo-2-((5-methoxy-2-methyl-4-(4-(4-methylpiperazin-1-yl)piperidin-1-yl)phenyl)amino)pyrimidin-4-yl)amino)-5-(4-methoxy-1H-pyrazol-1-yl)phenyl)dimethylphosphine oxide

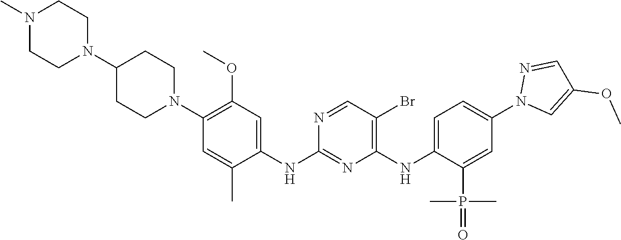

### Step 1: Synthesis of Compound K-018-2

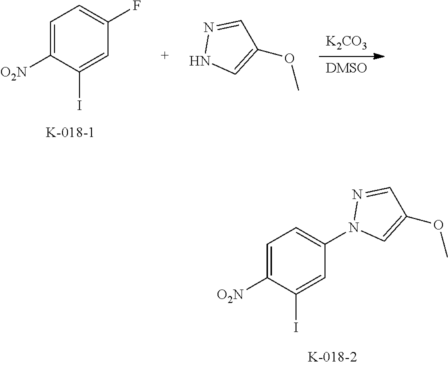

K-018-1 (300 mg) and 4-methoxy-1H-pyrazole (302 mg) was dissolved in DMSO (8 mL), K₂CO₃ (250 mg) was added to the solution at room temperature; the gas in reactor was replaced with nitrogen for three times. After well stirring, the solution was transferred to an oil bath, heated to 100° C., and stirred at 100° C. for 16 hr. The reaction was complete as monitored by LCMS. The reaction solution was cooled down to room temperature, poured into water, and extracted with ethyl acetate (3×50 mL); the organic phase was washed with saturated brine (3×30 mL), dried over anhydrous sodium sulfate, and purified by column chromatography (n-hexane:ethyl acetate=0%-80%). The solvent was removed to afford the desired product K-018-2 (50 mg), MS: 345.96 [M+H]⁺

### Step 2: Synthesis of Compound K-018-3

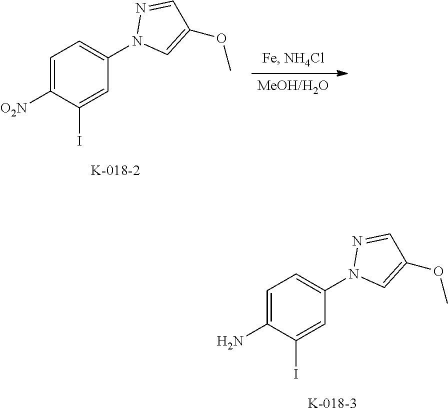

Dissolve K-018-2 (150 mg) in MeOH (8 mL) and H₂O (8 mL), add Fe powder (205 mg) and NH₄Cl (205 mg) to the solution at room temperature; the gas in the reactor was replaced with nitrogen for three times. After well stirring, the solution was transferred to an oil bath, heated to 80° C., and stirred under 80° C. for 3 hr. The reaction was complete as monitored by LCMS. The reaction solution was cooled down to room temperature, poured into water, extracted with DCM/MeOH=10:1 (3×50 mL), the organic phase was washed with saturated brine (3×30 mL), dried over anhydrous sodium sulfate, and purified by column chromatography (DCM/MeOH=0%-10%). The solvent was removed to afford the desired product K-018-3 (180 mg), MS: 315.99 [M+H]⁺

### Step 3: Synthesis of Compound K-018-4

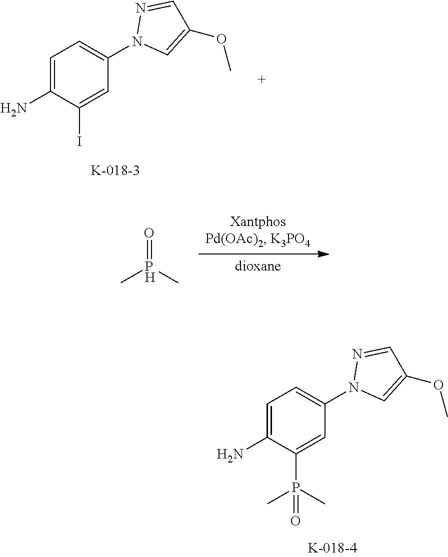

Add compound K-018-3 (3.0 g), dimethylphosphine oxide (865 mg), K₃PO₄ (6.41 g), Pd(OAc)₂ (226 mg), Xantphos (1.75 g), 1,4-Dioxane (60 mL) into the reaction flask, heat to 120° C. under N₂ protection, heat and stir for 16 hr. The reaction was complete as monitored by LCMS and then stopped. The reaction solution was poured into water (50 mL), extracted with dichloromethane (3×50 mL), the organic phase was washed with saturated brine (3×30 mL), dried over anhydrous sodium sulfate, separated and dried by column chromatography (dichloromethane:methanol=15:1), and concentrated to afford the desired yellow solid product K-0184 (2.0 g), MS: 266.10 [M+H]⁺

### Step 4: Synthesis of Compound K-018-5

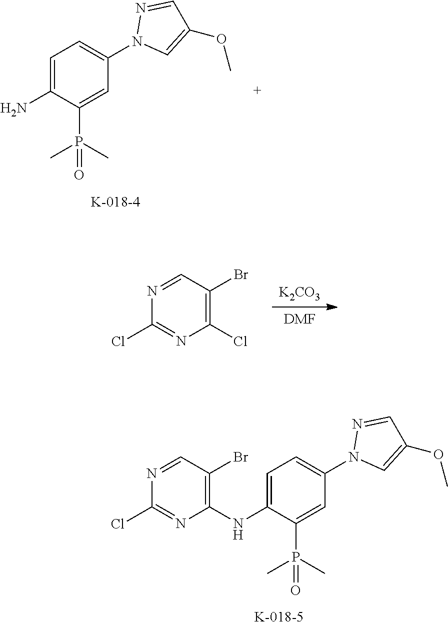

K-018-4 (1.0 g), 5-Bromo-2,4-dichloropyrimidine (1.39 g), K₂CO₃ (1.69 g) and DMF (20 mL) was added to the reaction flask, heated to 90° C., and stirred for 4 hr. The reaction was complete as monitored by LCMS and then stopped. The reaction solution was poured into water (50 mL), extracted with ethyl acetate (3×50 mL), the organic phase was washed with saturated brine (3×30 mL), dried over anhydrous sodium sulfate, separated and purified by column chromatography (dichloromethane:methanol=13:1). The solvent was removed to obtain the desired yellow solid compound K-018-5 (700 mg), MS: 455.99 [M+H]⁺

### Step 5: Synthesis of Compound K-018-8

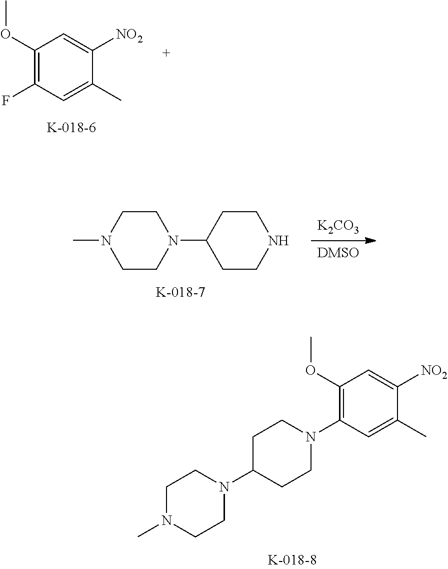

Add K-018-6 (3.00 g), K-018-7 (4.14 g), K₂CO₃ (6.24 g) and DMSO (30 ml) to the reaction flask, heat to 90° C., and stir with heating for 12 hr. The reaction was complete as monitored by LCMS and then stopped. The reaction solution was poured into water (100 ml), filtered by suction: the filter cake was washed with water and dried to obtain the desired yellow solid product K-018-8 (4.20 g), MS: 349.22 [M+H]⁺

### Step 6: Synthesis of Compound K-018-9

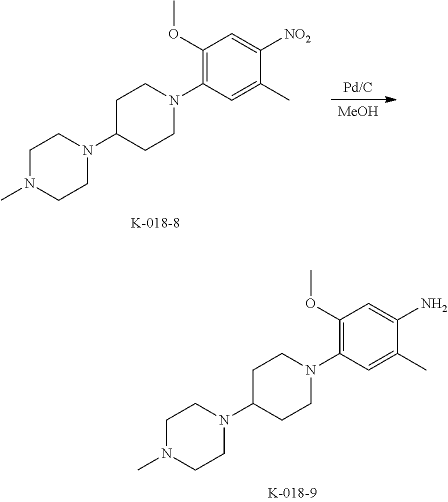

Add compound K-018-8 (4.20 g), Pd/C (1.00 g, 10%) and MeOH (60 ml) to the reaction flask, introduce H₂ and stir the solution at room temperature for 3 hr. The reaction was complete as monitored by LCMS and then stopped. The reaction solution was filtered by suction and rinsed with methanol (20 mL). The organic phase was collected and the solvent was removed to obtain a reddish brown liquid of the desired product compound K-018-9 (3.5 g), MS: 319.24 [M+H]⁺

### Step 7: Synthesis of Compound K-018

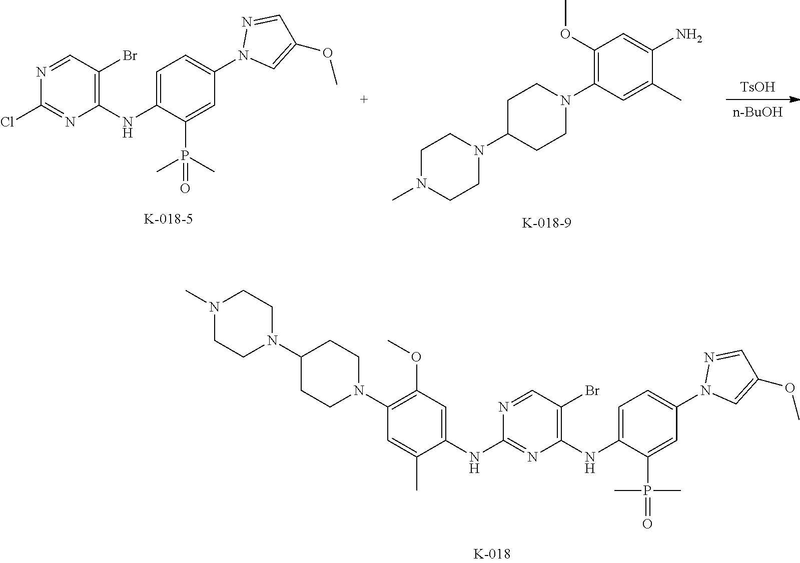

Add compound K-018-5 (100 mg), K-018-9 (76 mg), p-toluenesulfonic acid (60 mg) and n-butanol (2 ml) to the reaction flask, heat at 120° C., and stir for 12 hr. The reaction was complete as monitored by LCMS and then stopped. The reaction solution was poured into sodium carbonate water (5 ml) solution, extracted with dichloromethane (3×10 mL). The organic phase was washed with saturated brine (3×10 mL), dried over anhydrous sodium sulfate, separated and purified by column chromatography (dichloromethane:methanol=12:1), and concentrated to get the compound K-018 (60 mg) as an off-white solid, MS: 738.26 [M+H]⁺

### Example 2: Synthesis of Compound K-023

### (2-((5-Bromo-2-((5-methoxy-2-methyl-4-morpholinophenyl)amino)pyrimidin-4-yl)amino)-5-(4-methoxy-1H-pyrazol-1-yl)phenyl)dimethylphosphine oxide

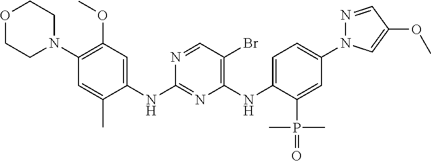 [SMILES: COc1cnn(-c2ccc(Nc3nc(Nc4cc(OC)c(N5CCOCC5)cc4C)ncc3Br)c(P(C)(C)=O)c2)c1]

### Step 1: Synthesis of Compound K-023-3

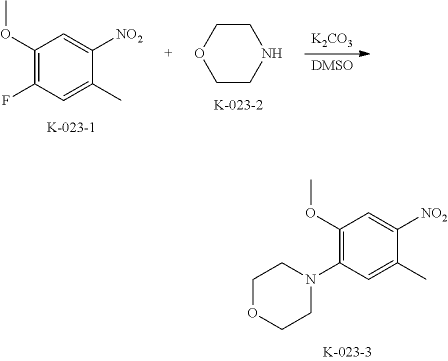

K-023-1 (300 mg) and K-023-2 (302 mg) was dissolved in DMF (8 mL), K₂CO₃ (250 mg) was added to the solution at room temperature; the gas in the reactor was replaced with nitrogen for three times. After well stirring, the solution was transferred to an oil bath, heated to 100° C., and stirred under 100° C. for 16 hr. The reaction was complete as monitored by LCMS. The reaction solution was cooled down to room temperature, poured into water (20 mL), extracted with ethyl acetate (3×20 ml). The organic phase was washed with saturated brine (3×10 mL), dried over anhydrous sodium sulfate, and separated and purified by column chromatography (n-hexane:ethyl acetate=0%-80%). The solvent was removed to obtain the desired product compound K-023-3 (250 mg), MS: 253.11 [M+H]⁺

### Step 2: Synthesis of Compound K-023-4

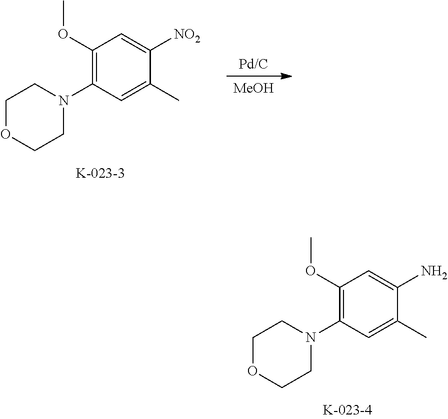 [SMILES: "MeOH" ; COc1cc([N+](=O)[O-])c(C)cc1N1CCOCC1 ; "Pd/C" ; COc1cc(N)c(C)cc1N1CCOCC1]

K-023-3 (4.20 g), Pd/C (1.00 g, 10%) and MeOH (60 mL) was added to the reaction flask, and stirred at room temperature for 3 hr under H₂. The reaction was complete as monitored by LCMS and then stopped. The solution was filtered by suction and rinsed with methanol (20 mL). The organic phase was collected and the solvent was removed to obtain desired product compound K-023-4 (3.5 g) as a reddish-brown liquid, MS: 223.14 [M+H]⁺

### Step 3: Synthesis of Compound K-023

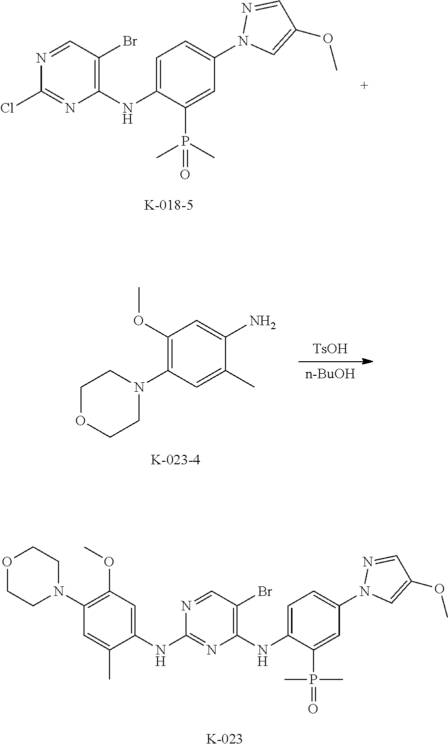

K-018-5 (100 mg), K-023-4 (76 mg), p-toluenesulfonic acid (60 mg) and n-butanol (2 mL) was added to the reaction flask, heated to 120° C. and stirred for 12 hr. The reaction was complete as monitored by LCMS and then stopped. The reaction solution was poured into sodium carbonate water (5 mL) solution, and extracted with dichloromethane (3×50 mL); the organic phase was washed with saturated brine (3×30 mL), dried over anhydrous sodium sulfate, and separated and purified by column chromatography (dichloromethane:methanol=12:1) to obtain the compound K-023 (60 mg) as an off-white solid, MS: 642.15 [M+H]⁺

¹H NMR (500 MHz, DMSO-d6) δ 11.88 (s, 1H), 9.91 (s, 1H), 8.36-8.42 (m, 2H), 7.80-7.83 (d, 1H), 7.59 (s, 2H), 7.11 (s, 1H), 3.84 (s, 4H), 3.80 (s, 3H), 3.70 (s, 3H), 3.19 (s, 4H), 2.14 (s, 3H), 1.88 (s, 3H), 1.86 (s, 3H).

### Example 3: Synthesis of Compound K-028

### (2-((5-Bromo-2-((5-methoxy-2-methyl-4-(4-methylpiperazin-1-yl)phenyl)amino)pyrimidin-4-yl)amino)-5-(4-methoxy-1H-pyrazol-1-yl)phenyl)-dimethylphosphine oxide

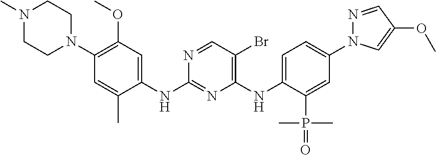

### Step 1: Synthesis of Compound K-028-3

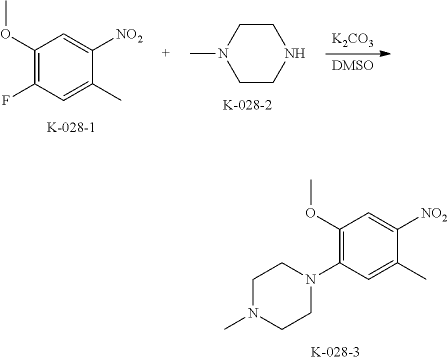

Dissolve K-028-1 (300 mg) and K-028-2 (302 mg) in DMF (8 mL), add K₂CO₃ (250 mg) at room temperature; the gas in reactor was replaced with nitrogen for three times; after well stirring, the solution was transferred to oil bath, heated to 100° C., and stirred at 100° C. for 16 hr. The reaction was complete as monitored by LCMS and then stopped. The reaction solution was cooled down to room temperature, poured into water (20 mL), and extracted with ethyl acetate (3×20 mL); the organic phase was washed with saturated brine (3×10 mL), dried over anhydrous sodium sulfate, separated and purified by column chromatography (n-hexane:ethyl acetate=0%-80%); the solvent was removed to obtain the desired product compound K-028-3 (250 mg), MS: 266.14 [M+H]⁺

### Step 2: Synthesis of Compound K-028-4

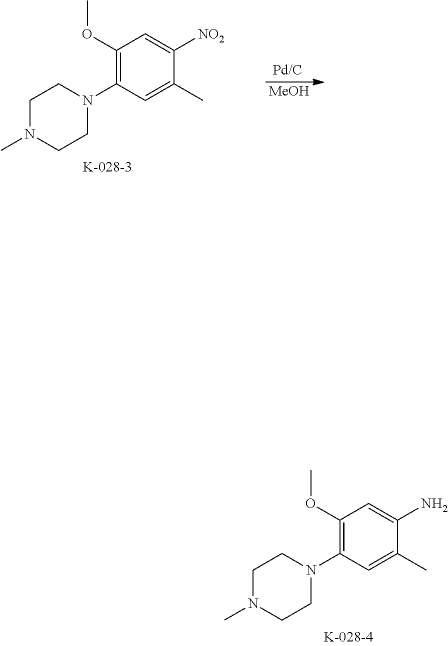

Add compound K-028-3 (4.20 g), Pd/C (1.00 g, 10%) and MeOH (60 ml) to the reaction flask, introduce H₂. The reaction solution was stirred at room temperature for 3 hr. The reaction was complete as monitored by LCMS and then stopped. The solution was filtered with suction, rinsed with methanol (20 mL); collect the organic phase, remove the solvent, and obtain the reddish-brown liquid of desired product compound K-028-4 (3.5 g), MS: 236.17 [M+H]⁺

### Step 3: Synthesis of Compound K-028

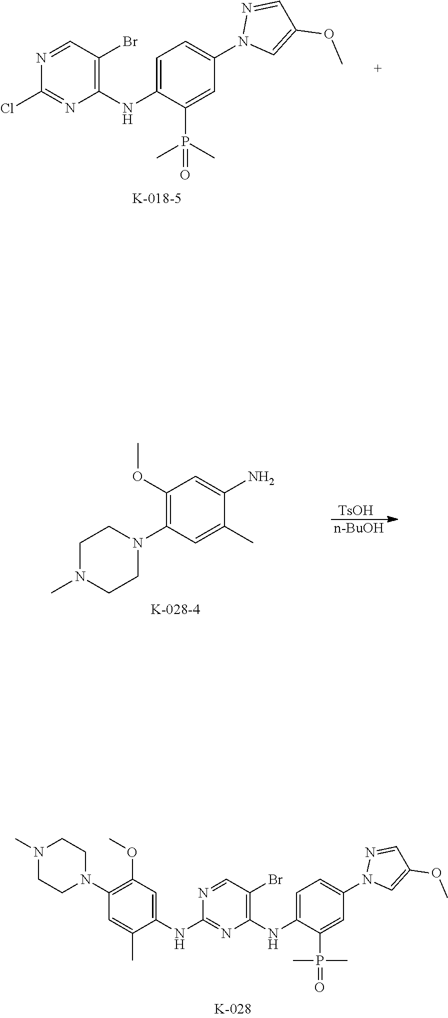

Add compound K-018-5 (100 mg), K-028-4 (76 mg), p-toluenesulfonic acid (60 mg) and n-butanol (2 ml) to the reaction flask, heat to 120° C., and stir for 12 hr. The reaction was complete as monitored by LCMS and then stopped. The reaction solution was poured into sodium carbonate water (5 mL) solution, and extracted with dichloromethane (3×50 mL); the organic phase was washed with saturated brine (3×30 mL), dried over anhydrous sodium sulfate, separated and purified by column chromatography (dichloromethane:methanol=12:1), and concentrated to obtain the off-white solid of compound K-028 (60 mg), MS: 655.18 [M+H]⁺

¹H NMR (500 MHz, DMSO-d6) δ 12.12 (s, 1H), 11.04 (s, 1H), 10.20 (s, 1H), 8.45 (s, 2H), 7.83-7.85 (d, 1H), 7.67 (s, 1H), 7.55 (s, 1H), 7.07 (s, 1H), 6.89 (s, 1H), 3.80 (s, 3H), 3.68 (s, 3H), 3.48-3.56 (dd, 4H), 3.19-3.25 (q, 2H), 3.06-3.11 (t, 2H), 2.83-2.84 (d, 3H), 2.14 (s, 3H), 1.91 (s, 3H), 1.88 (s, 3H).

### Example 4: Synthesis of Compound K-039

### (2-((5-Bromo-2-((4-(4-hydroxypiperidin-1-yl)-5-methoxy-2-methylphenyl)amino)pyrimidin-4-yl)amino)-5-(4-methoxy-1H-pyrazol-1-yl)phenyl)dimethylphosphine oxide

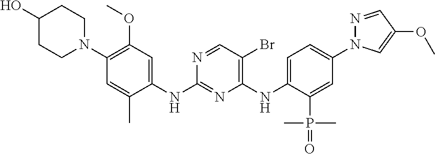 [SMILES: COc1cnn(-c2ccc(Nc3nc(Nc4cc(OC)c(N5CCC(O)CC5)cc4C)ncc3Br)c(P(C)(C)=O)c2)c1]

### Step 1: Synthesis of Compound K-039-3

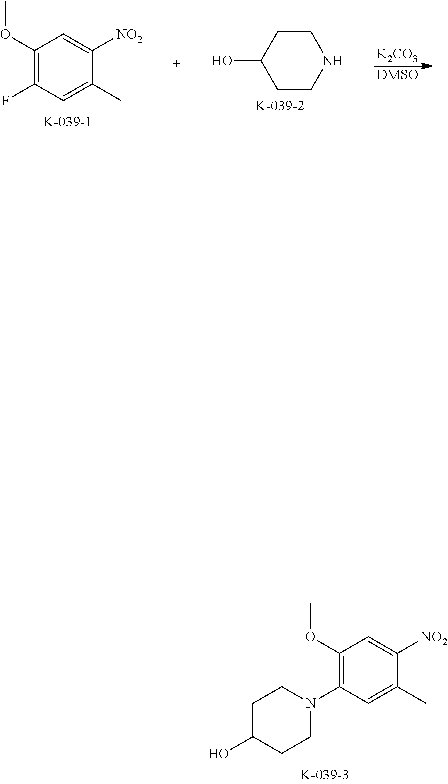

Dissolve K-039-1 (300 mg) and K-039-2 (302 mg) in DMF (8 mL), add K₂CO₃ (250 mg) to the solution at room temperature; the gas in reactor was replaced with nitrogen for three times. After well stirring, the solution was transferred to oil bath, heated to 100° C. and stirred under 100° C. for 16 hr. The reaction was complete as monitored by LCMS. The reaction solution was cooled down to room temperature, poured into water (20 mL), and extracted with ethyl acetate (3×20 mL); the organic phase was washed with saturated brine (3×10 mL), dried over anhydrous sodium sulfate, separated and purified by column chromatography (n-hexane:ethyl acetate=0%-80%); the solvent was removed to obtain the desired product compound K-039-3 (250 mg), MS: 267.13 [M+H]⁺

### Step 2: Synthesis of Compound K-039-4

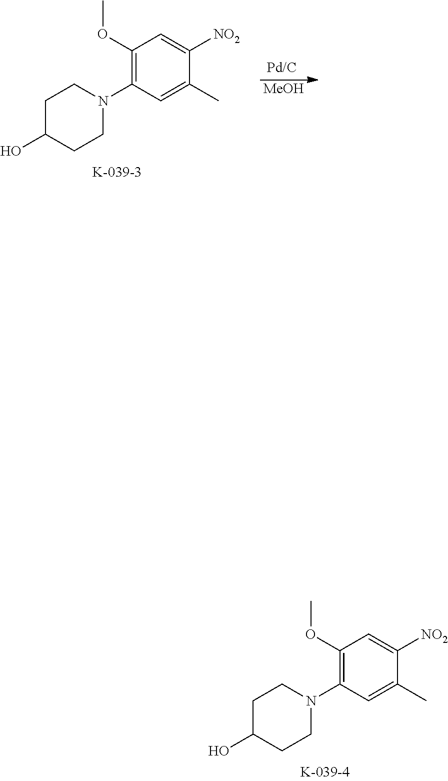 [SMILES: "MeOH" ; COc1cc([N+](=O)[O-])c(C)cc1N1CCC(O)CC1 ; COc1cc([N+](=O)[O-])c(C)cc1N1CCC(O)CC1]

Add compound K-039-3 (4.20 g), Pd/C (1.00 g, 10%) and MeOH (60 mL) to the reaction flask, introduce H₂; the reaction solution was stirred at room temperature for 3 hr. The reaction was complete as monitored by LCMS, and then stopped. The solution was filtered with suction, and rinsed with methanol (20 mL); collect the organic phase, remove the solvent, and obtain the reddish-brown liquid of desired product compound K-039-4 (3.5 g), MS: 237.16 [M+H]⁺

### Step 2: Synthesis of Compound K-039

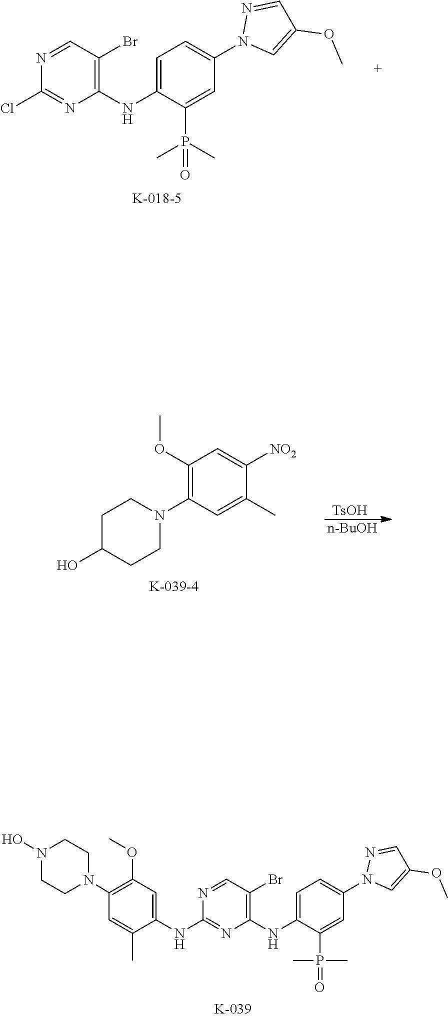

Add compound K-018-5 (100 mg), K-039-4 (76 mg), p-toluenesulfonic acid (60 mg) and n-butanol (2 mL) to the reaction flask, heat to 120° C. and stir for 12 hr. The reaction was complete as monitored by LCMS, and then stopped. The reaction solution was poured into sodium carbonate water (5 mL) solution, and extracted with dichloromethane (3×50 mL); the organic phase was washed with saturated brine (3×30 mL), dried over anhydrous sodium sulfate, separated and purified by column chromatography (dichloromethane:methanol=12:1), and concentrated to obtain the off-white solid compound K-039 (60 mg), MS: 656.17 [M+H]⁺

### Example 5: Synthesis of Compound K-026

### (2-((5-Bromo-2-((2-cyclopropyl-5-methoxy-4-morpholinophenyl)amino)pyrimidin-4-yl)amino)-5-(4-methoxy-1H-pyrazol-1-yl)phenyl)dimethylphosphine oxide

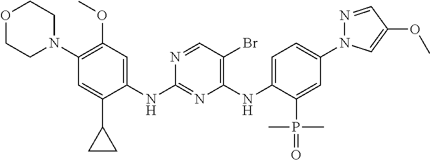 [SMILES: COc1cnn(-c2ccc(Nc3nc(Nc4cc(OC)c(N5CCOCC5)cc4C4CC4)ncc3Br)c(P(C)(C)=O)c2)c1]

### Step 1: Synthesis of Compound K-026-2

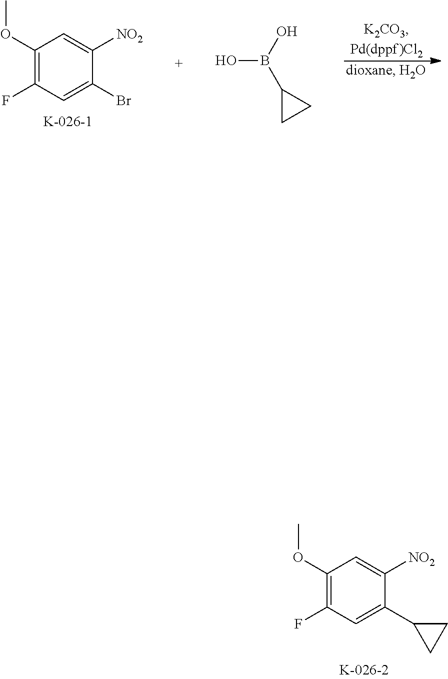

K-026-1 (300 mg) and cyclopropylboronic acid (302 mg) were dissolved in 1,4-dioxane (8 mL) in a reactor equipped with a stir bar and nitrogen protector, K₂CO₃ (250 mg) and Pd(dppf)Cl₂ (200 mg) were added to the solution at room temperature, and the gas in reactor was replaced with nitrogen for three times. After well stirring, the solution was transferred to an oil bath, heated to 100° C., and stirred at 100° C. for 16 hr. The reaction was complete as monitored by TLC. The reaction solution was cooled down to room temperature, poured into water (20 mL), and extracted with ethyl acetate (3×20 mL); the organic phase was washed with saturated brine (3×10 mL), dried over anhydrous sodium sulfate, separated and purified by column chromatography (n-hexane:ethyl acetate=0%-80%), and the solvent was removed to obtain the desired product compound K-026-2 (250 mg).

### Step 2: Synthesis of Compound K-026-4

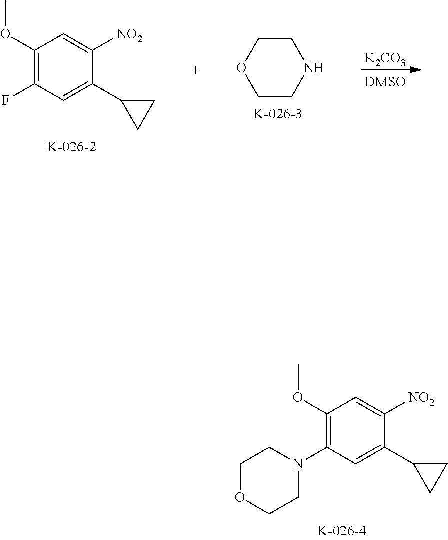

Dissolve K-026-2 (300 mg) and K-026-3 (302 mg) in DMSO (8 mL), K₂CO₃ (250 mg) was added at room temperature, and the gas in reactor was replaced with nitrogen for three times. After well stirring, the solution was transferred to an oil bath, heated to 100° C., and stirred at 100° C. for 16 hr. The reaction was complete as monitored by LCMS. The reaction solution was cooled down to room temperature, poured into water (20 mL), and extracted with ethyl acetate (3×20 mL); the organic phase was washed with saturated brine (3×10 mL), dried over anhydrous sodium sulfate, separated and purified by column chromatography (n-hexane:ethyl acetate-0%-80%), and the solvent was removed to obtain the desired product compound K-026-4 (250 mg), MS: 279.13 [M+H]+

### Step 3: Synthesis of Compound K-026-5

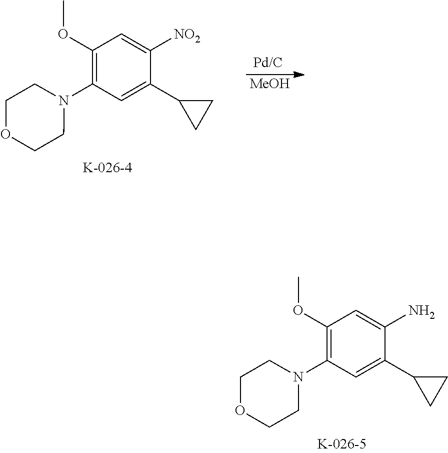 [SMILES: "MeOH" ; COc1cc([N+](=O)[O-])c(C2CC2)cc1N1CCOCC1 ; COc1cc(N)c(C2CC2)cc1N1CCOCC1]

Add compound k-026-4 (4.20 g), Pd/C (1.00 g, 10%) and MeOH (60 ml) into the reaction flask, introduce H₂; the reaction solution was stirred at room temperature for 3 hr. The reaction was complete as monitored by LCMS, and then stopped. The solution was filtered with suction, and rinsed with methanol (20 mL); collect the organic phase, remove the solvent, and obtain the reddish-brown liquid of desired product compound K-026-5 (3.5 g), MS: 249.15 [M+H]+

### Step 4: Synthesis of Compound K-026

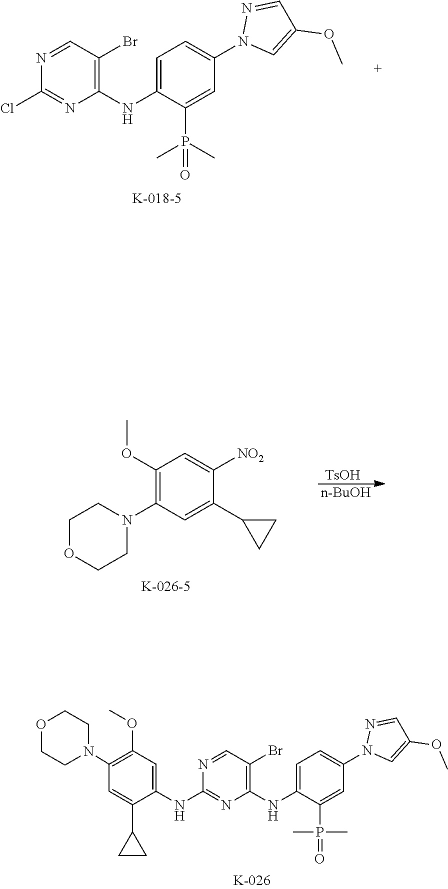

Add compounds K-018-5 (100 mg), k-026-5 (76 mg), p-toluenesulfonic acid (60 mg) and n-butanol (2 ml) to the reaction flask, heat to 120° C., and stir for 12 hr. The reaction was complete as monitored by LCMS, and then stopped. The reaction solution was poured into sodium carbonate water (5 ml) solution, and extracted with dichloromethane (3×50 mL); the organic phase was washed with saturated brine (3×30 mL), dried over anhydrous sodium sulfate, separated and purified by column chromatography (dichloromethane:methanol=12:1), and concentrated to obtain the off-white solid compound K-026 (60 mg), MS: 668.17 [M+H]+

### Example 6: Synthesis of Compound K-020

### (2-((2-((4-[1,4′-Bipiperidin]-1-yl)-5-ethyl-2-methoxyphenyl)amino)-5-bromopyrimidin-4-yl)amino)-5-(4-methoxy-1H-pyrazol-1-yl)phenyl)dimethylphosphine oxide

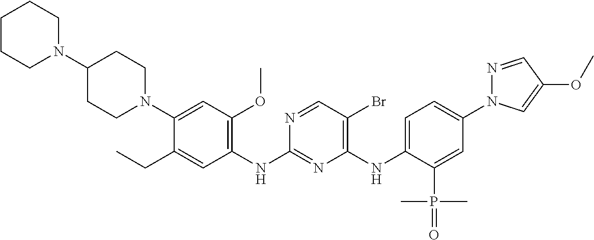 [SMILES: CCc1cc(Nc2ncc(Br)c(Nc3ccc(-n4cc(OC)cn4)cc3P(C)(C)=O)n2)c(OC)cc1N1CCC(N2CCCCC2)CC1]

### Step 1: Synthesis of Compound K-020-3

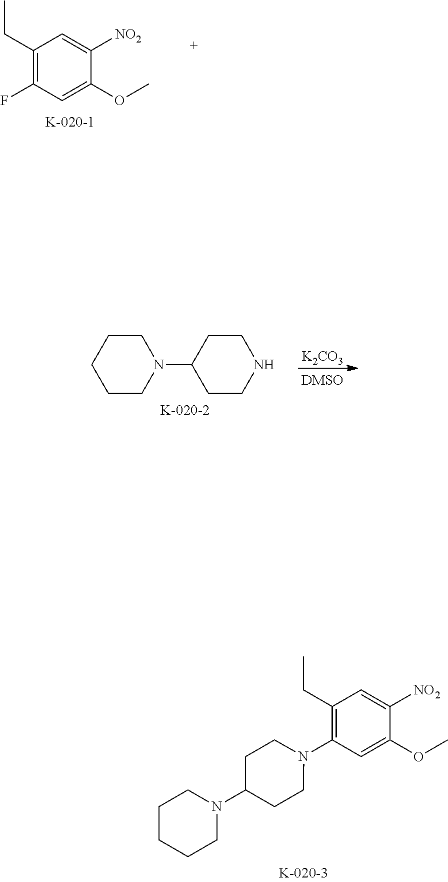

K-020-1 (300 mg) and K-020-2 (302 mg) were dissolved in DMF (8 mL) in a reactor equipped with a stir bar and nitrogen protector. K₂CO₃ (250 mg) were added to the solution at room temperature, and the gas in reactor was replaced with nitrogen for three times. After well stirring, the solution was transferred to an oil bath, heated to 100° C. and stirred at 100° C. for 16 hr. The reaction was complete as monitored by LCMS. The reaction solution was cooled down to room temperature, poured into water (20 mL), and extracted with ethyl acetate (3×20 mL); the organic phase was washed with saturated brine (3×10 mL), dried over anhydrous sodium sulfate, separated and purified by column chromatography (n-hexane:ethyl acetate=0%-80%), and the solvent was removed to obtain the desired product compound K-020-3 (250 mg), MS: 348.22 [M+H]⁺

### Step 2: Synthesis of Compound K-020-4

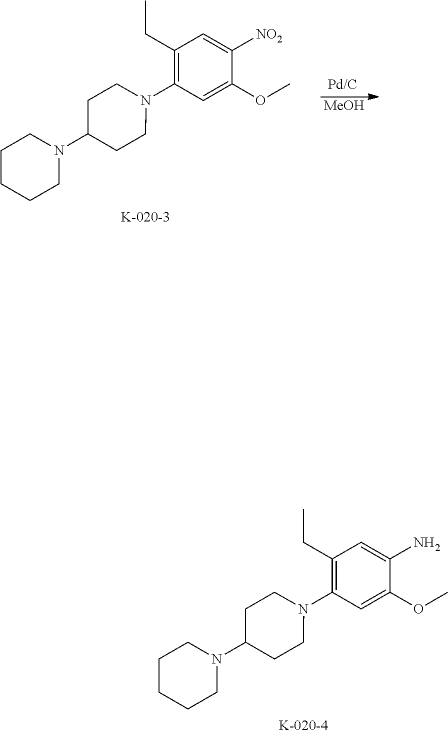 [SMILES: "MeOH" ; CCc1cc([N+](=O)[O-])c(OC)cc1N1CCC(N2CCCCC2)CC1 ; CCc1cc(N)c(OC)cc1N1CCC(N2CCCCC2)CC1]

Add compound K-020-3 (4.20 g), Pd/C (1.00 g, 10%) and MeOH (60 mL) into the reaction flask, introduce H₂; the reaction solution was stirred at room temperature for 3 hr. The reaction was complete as monitored by LCMS, and then stopped. The solution was filtered with suction, and rinsed with methanol (20 mL); collect the organic phase, remove the solvent, and obtain the reddish-brown liquid of desired product compound K-020-4 (3.5 g), MS: 318.25 [M+H]⁺

### Step 3: Synthesis of Compound K-020

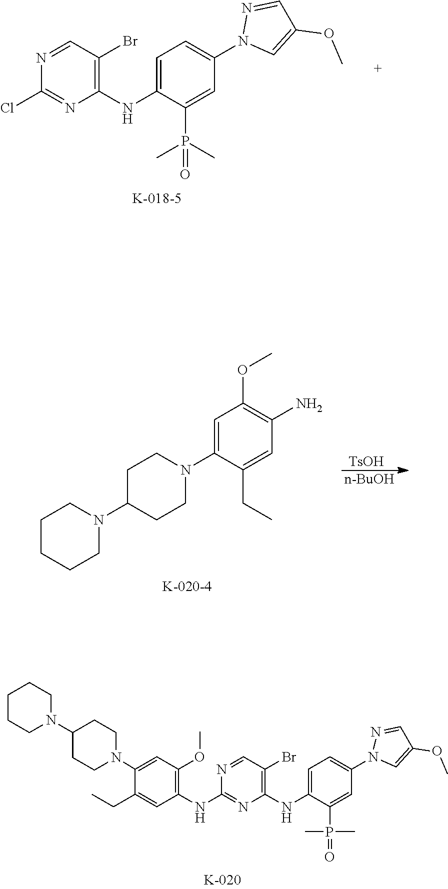

Add compounds K-018-5 (100 mg), K-020-4 (76 mg), p-toluenesulfonic acid (60 mg) and n-butanol (2 ml) to the reaction flask, heat to 120° C., and stir for 12 hr. The reaction was complete as monitored by LCMS, and then stopped. The reaction solution was poured into sodium carbonate water (5 mL) solution, and extracted with dichloromethane (3×50 mL); the organic phase was washed with saturated brine (3×30 mL), dried over anhydrous sodium sulfate, separated and purified by column chromatography (dichloromethane:methanol=12:1), and concentrated to obtain the off-white solid compound K-020 (60 mg), MS: 737.26 [M+H]+

¹H NMR (500 MHz, DMSO-d6) δ 10.90 (s, 1H), 9.73 (s, 1H), 8.31 (s, 1H), 8.35 (s, 1H), 8.22 (s, 1H), 8.18 (s, 1H), 7.75-7.78 (dd, 1H), 7.70-7.72 (d, 1H), 7.62 (s, 1H), 7.36 (s, 1H), 3.79 (s, 3H), 3.75 (s, 3H), 3.47 (br, 2H), 3.34 (s, 6H), 3.24 (br, 1H), 3.10 (br, 2H), 2.98 (br, 2H), 2.76 (m, 2H), 2.14 (m, 2H), 1.85 (s, 3H), 1.82 (s, 3H), 1.42 (br, 2H), 0.99-1.02 (t, 3H).

### Example 7: Synthesis of Compound K-009

### (2-((5-Bromo-2-((5-ethyl-2-methoxy-4-(4-(4-methylpiperazin-1-yl)piperidin-1-yl)phenyl)amino)pyrimidin-4-yl)amino)-5-(4-ethynyl-1H-pyrazol-1-yl)phenyl)dimethylphosphine oxide

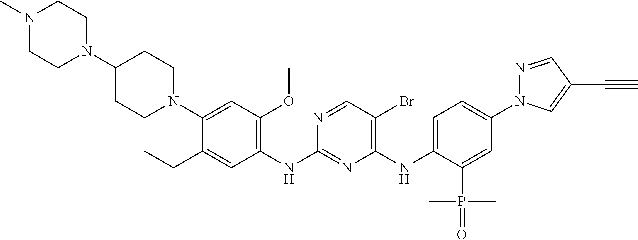 [SMILES: C#Cc1cnn(-c2ccc(Nc3nc(Nc4cc(CC)c(N5CCC(N6CCN(C)CC6)CC5)cc4OC)ncc3Br)c(P(C)(C)=O)c2)c1]

### Step 1: Synthesis of Compound K-009-2

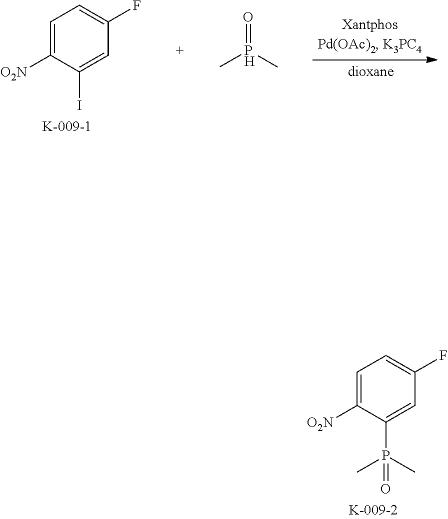

Add compound K-009-1 (3.0 g), dimethylphosphine oxide (865 mg), K₃PO₄ (6.41 g), Pd(OAc)₂ (226 mg), Xantphos (1.75 g), 1,4-Dioxane (60 mL) to the reaction flask, heat to 120° C. under N₂ protection, and stir with heating for 16 hr. The reaction was complete as monitored by LCMS, and then stopped. The reaction solution was poured into water (50 mL) solution, and extracted with dichloromethane (3×50 mL); the organic phase was washed with saturated brine (3×30 mL), dried over anhydrous sodium sulfate, separated and purified by column chromatography (dichloromethane:methanol=15:1), then concentrated and dried to obtain the yellow solid of desired product compound K-009-2 (2.0 g), MS: 218.03 [M+H]⁺

### Step 2: Synthesis of Compound K-009-3

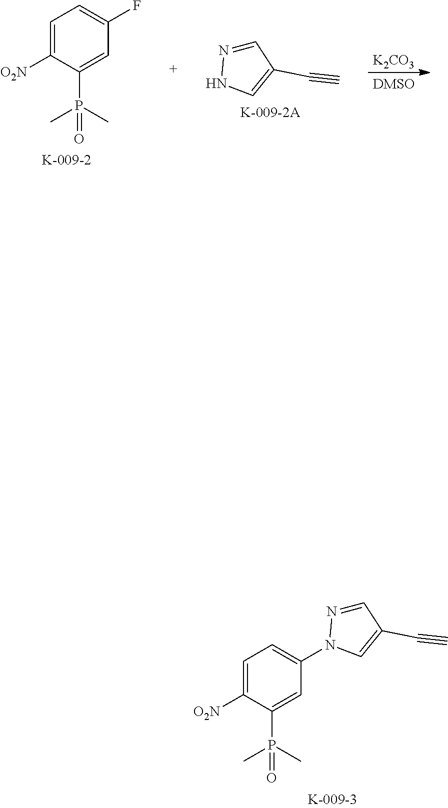

K-009-2 (300 mg) and K-009-2A (302 mg) were dissolved in DMF (8 mL). K₂CO₃ (250 mg) were added to the solution at room temperature, and the gas in reactor was replaced with nitrogen for three times. After well stirring, the solution was transferred to an oil bath, heated to 100° C., and stirred at 100 for 16 hr. The reaction was complete as monitored by LCMS. The reaction solution was cooled down to room temperature, poured into water, and extracted with 30 mL ethyl acetate for three times; the organic phases were concentrated by rotary evaporation. Silica gel was added to the concentrated organic phase to mix the sample, and then separated and purified from 0%-80% PE:EtOAc column by gradient, and then concentrated and dried to obtain 250 mg of compound K-009-3, MS: 290.06 [M+H]⁺

### Step 3: Synthesis of Compound K-009-4

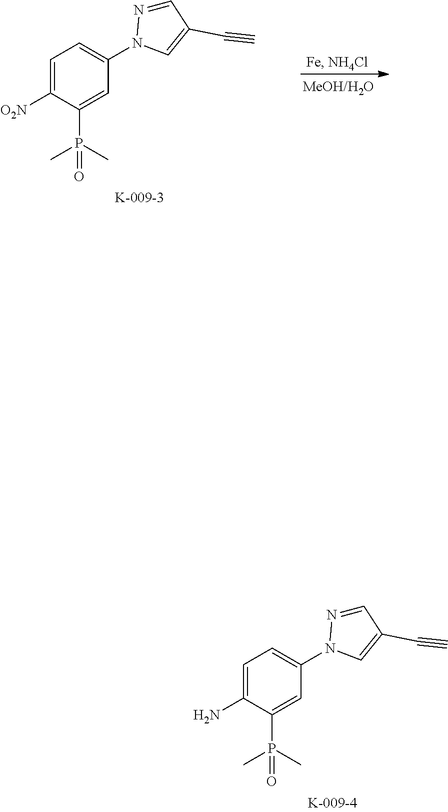

K-009-3 (150 mg) was dissolved in MeOH (8 mL) and H₂O (8 mL) in a reactor equipped with a stir bar and nitrogen protector, Fe powder (205 mg) and NH₄Cl (205 mg) were added to the solution at room temperature, and the gas in reactor was replaced with nitrogen for three times. After well stirring, the solution was transferred to an oil bath, heated to 80° C. and stirred at 80° C. for 3 hr. The reaction was complete as monitored by LCMS. The reaction solution was cooled down to room temperature, poured into water (20 mL), and extracted with ethyl acetate (3×20 mL); the organic phase was washed with saturated brine (3×10 mL), dried over anhydrous sodium sulfate, separated and purified by column chromatography (n-hexane:ethyl acetate-0%-80%), and the solvent was removed to obtain the desired product compound K-009-4 (180 mg), MS: 260.09 [M+H]⁺

### Step 4: Synthesis of Compound K-009-5

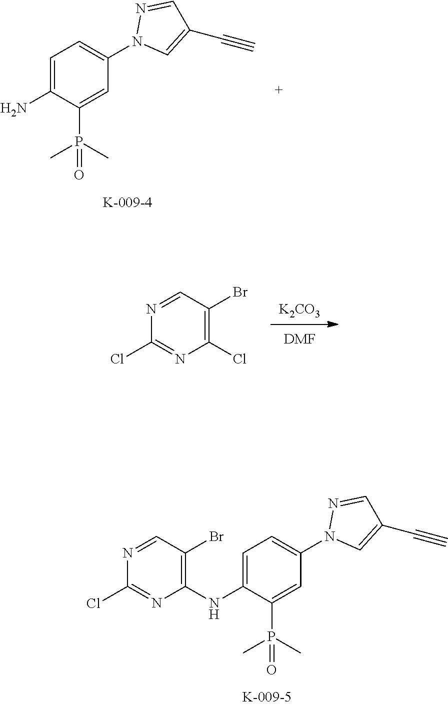

Add compounds K-009-4 (1.0 g), 5-Bromo-2,4-dichloropyrimidine (1.39 g). K₂CO₃ (1.69 g) and DMF (20 ml) to the reaction flask, heat to 90° C., and stir with heating for 4 hr. The reaction was complete as monitored by LCMS, and then stopped. The reaction solution was poured into water (50 mL), and extracted with ethyl acetate (3×50 mL); the organic phase was washed with saturated brine (3×30 mL), dried over anhydrous sodium sulfate, separated and purified by column chromatography (dichloromethane:methanol=13:1), and the solvent was removed to obtain the yellow solid of desired product compound K-009-5 (700 mg), MS: 449.98 [M+H]+

### Step 5: Synthesis of Compound K-009-8

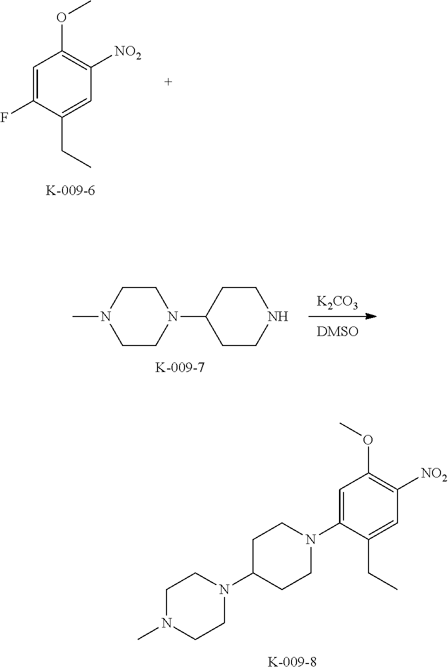

Add compounds K-009-6 (3.00 g), K-009-7 (4.14 g), K₂CO₃ (6.24 g) and DMSO (30 mL) to the reaction flask, heat to 90° C., and stir with heating for 12 hr. The reaction was complete as monitored by LCMS, and then stopped. The reaction solution was poured into water (100 mL), and filtered with suction; the filter cake was washed with water and dried to obtain yellow solid of desired product compound K-009-8 (4.20 g), MS: 363.23 [M+H]⁺

### Step 6: Synthesis of Compound K-009-9

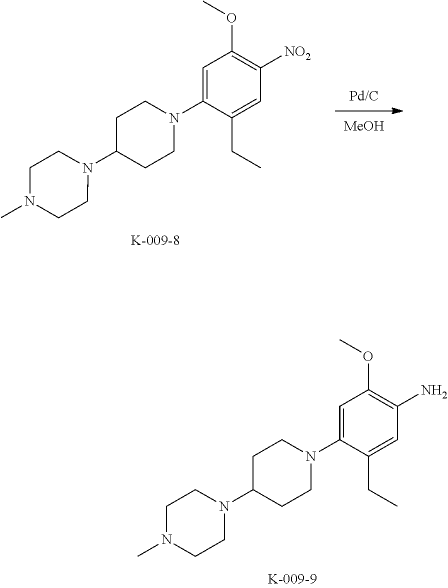 [SMILES: "MeOH" ; CCc1cc([N+](=O)[O-])c(OC)cc1N1CCC(N2CCN(C)CC2)CC1 ; "Pd/C" ; CCc1cc(N)c(OC)cc1N1CCC(N2CCN(C)CC2)CC1]

Add compound K-009-8 (4.20 g), Pd/C (1.00 g, 10%) and MeOH (60 mL) into the reaction flask, introduce H₂; the reaction solution was stirred at room temperature for 3 hr. The reaction was complete as monitored by LCMS, and then stopped. The solution was filtered with suction, and rinsed with methanol (20 mL); collect the organic phase, remove the solvent, and obtain the reddish-brown liquid of desired product compound K-009-4 (3.5 g), MS: 333.26 [M+H]⁺

### Step 7: Synthesis of Compound K-009

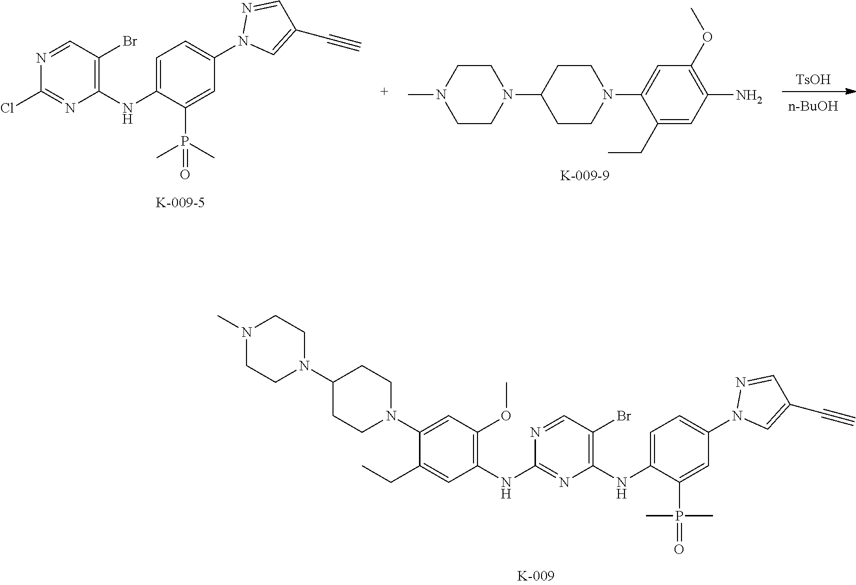

Add compound K-009-5 (100 mg), 7-1 (76 mg), p-toluenesulfonic acid (60 mg) and n-butanol (2 mL) to the reaction flask, heat to 120° C., and stir for 12 hr. The reaction was complete as monitored by LCMS, and then stopped. The reaction solution was poured into sodium carbonate water solution (5 ml), and extracted with dichloromethane (3×50 mL); the organic phase was washed with saturated brine (3×30 mL), dried over anhydrous sodium sulfate, separated and purified by column chromatography (dichloromethane:methanol=12:1), concentrated and dried to obtain off-white solid of desired product compound K-009 (60 mg), MS: 746.26 [M+H]⁺

### Example 8: Synthesis of Compound K-004

(2-((5-Bromo-2-((5-ethyl-2-methoxy-4-(4-(4-methylpiperazin-1-yl)piperidin-1-yl)phenyl)amino)pyrimidin-4-yl)amino)-5-(4-(methoxymethyl)-1H-pyrazol-1-yl)phenyl)dimethylphosphine oxide

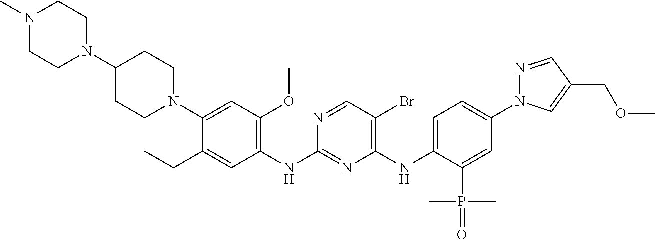

### Step 1: Synthesis of Compound K-004-2

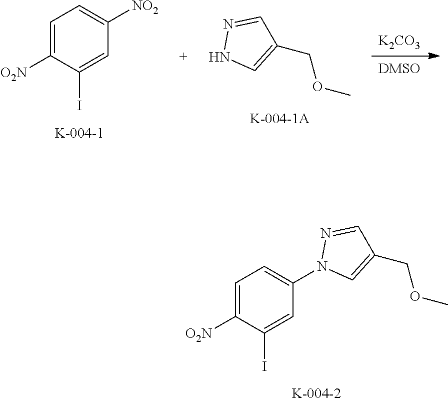

Dissolve K-004-1 (300 mg) and K-004-1A (302 mg) in DMSO (8 mL), K₂CO₃ (250 mg) were added to the solution at room temperature, and the gas in reactor was replaced with nitrogen for three times. After well stirring, the solution was transferred to an oil bath, heated to 100° C., and stirred at 100° C. for 16 hr. The reaction was complete as monitored by LCMS. The reaction solution was cooled down to room temperature, poured into water (50 mL), and extracted with ethyl acetate (3×50 mL); the organic phase was washed with saturated brine (3×30 mL), dried over anhydrous sodium sulfate, separated and purified by column chromatography (dichloromethane:methanol=15:1), then concentrated and dried to obtain a yellow solid of desired product compound K-004-2 (250 mg), MS: 359.98 [M+H]⁺

### Step 2: Synthesis of Compound K-004-3

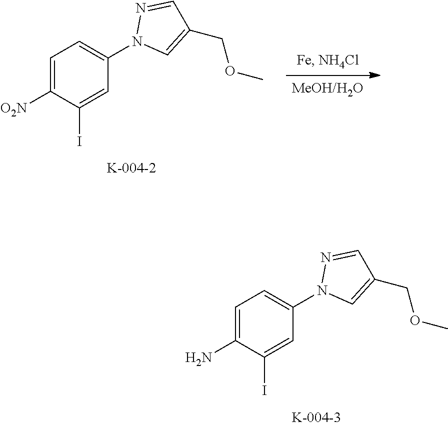

K-004-2 (150 mg) was dissolved in MeOH (8 ml) and H₂O (8 ml) in a reactor equipped with a stir bar and nitrogen protector, Fe powder (205 mg) and NH₄Cl (205 mg) were added to the solution at room temperature, and the gas in reactor was replaced with nitrogen for three times. After well stirring, the solution was transferred to an oil bath, heated to 80° C., and stirred at 80° C. for 3 hr. The reaction was complete as monitored by LCMS. The reaction solution was cooled down to room temperature, poured into water (50 mL), and extracted with dichloromethane (3×50 ml); the organic phase was washed with saturated brine (3×30 mL), dried over anhydrous sodium sulfate, separated and purified by column chromatography (dichloromethane:methanol=15:1), then concentrated and dried to obtain a yellow solid of desired product compound K-004-3 (180 mg), MS: 330.00 [M+H]+

### Step 3: Synthesis of Compound K-004-4

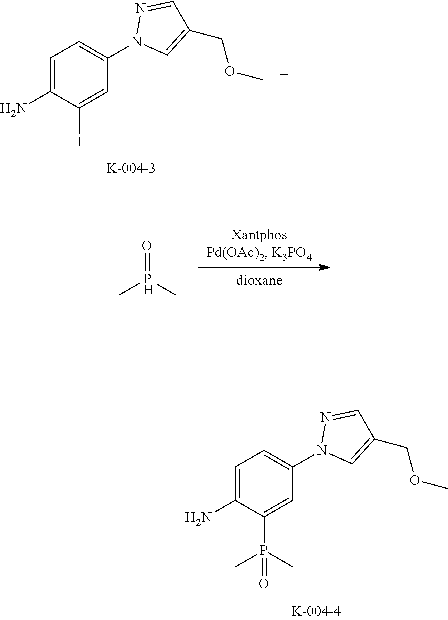

Add compound K-004-3 (3.0 g), dimethylphosphine oxide (865 mg), K₃PO₄ (6.41 g), Pd(OAc)₂ (226 mg), Xantphos (1.75 g), 1,4-Dioxane (60 mL) to the reaction flask, heat to 120° C. under nitrogen protection, and stir with heating for 16 hr. The reaction was complete as monitored by LCMS, and then stopped. The reaction solution was poured into water (50 mL), and extracted with dichloromethane (3×50 mL); the organic phase was washed with saturated brine (3×30 mL), dried over anhydrous sodium sulfate, separated and purified by column chromatography (dichloromethane:methanol=15:1), then concentrated and dried to obtain a yellow solid of desired product compound K-004-4 (2.0 g), MS: 280.11 [M+H]+

### Step 4: Synthesis of Compound K-004-5

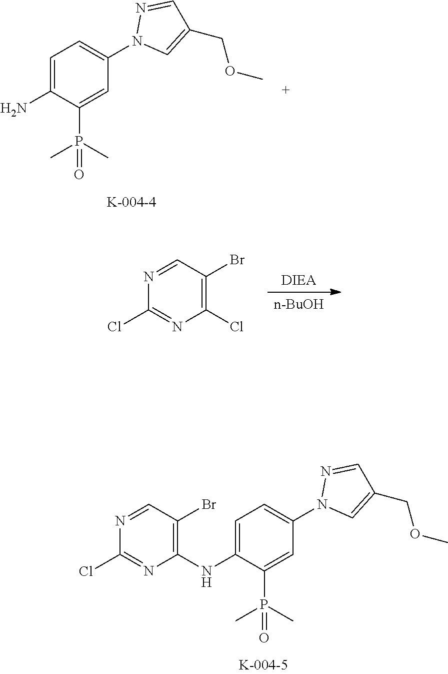

Add compound K-004-4 (1.0 g), 5-Bromo-2,4-dichloropyrimidine (1.39 g), DIEA (1.69 g) and n-BuOH (20 mL) to the reaction flask, heat to 130° C., and stir with heating for 36 hr. The reaction was complete as monitored by LCMS, and then stopped. The reaction solution was poured into water (50 mL), and extracted with ethyl acetate (3×50 mL); the organic phase was washed with saturated brine (3×30 mL), dried over anhydrous sodium sulfate, separated and purified by column chromatography (dichloromethane:methanol=13:1), and the solvent was removed to obtain a yellow solid of desired product compound K-004-5 (700 mg), MS: 471.01 [M+H]+

### Step 5: Synthesis of Compound K-004

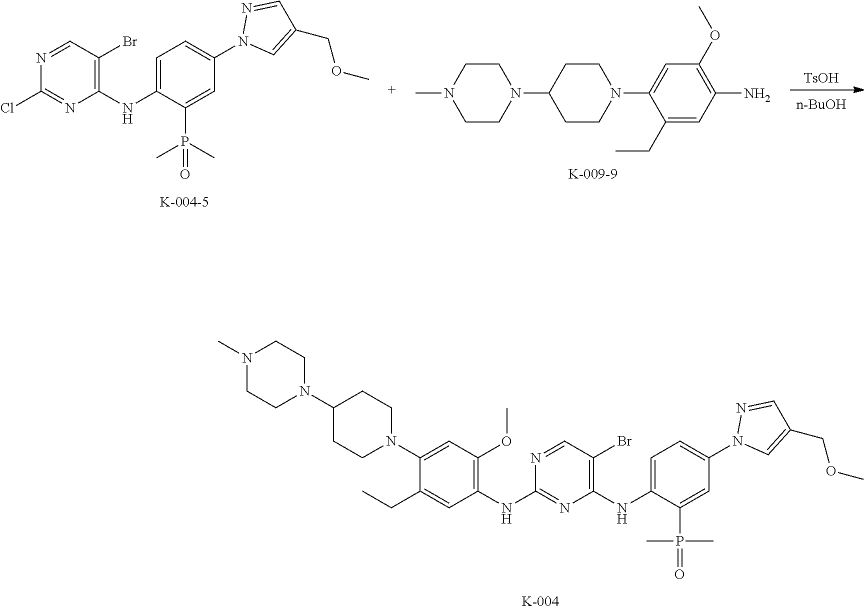

Add compounds K-004-5 (100 mg), K-009-9 (76 mg), p-toluenesulfonic acid mg) and n-butanol (2 mL) to the reaction flask, heat to 120° C., and stir for 12 hr. The reaction was complete as monitored by LCMS, and then stopped. The reaction solution was poured into sodium carbonate water (5 mL) solution, and extracted with dichloromethane (3×50 mL); the organic phase was washed with saturated brine (3×30 mL), dried over anhydrous sodium sulfate, separated and purified by column chromatography (dichloromethane:methanol=12:1), and concentrated to obtain the off-white solid compound K-004 (60 mg), MS: 766.29 [M+H]+

### Example 9: Synthesis of Compound K-031

### (2-((5-Bromo-2-((3-methoxy-5-methyl-4-morpholinophenyl)amino)pyrimidin-4-yl)amino)-5-(4-methoxy-1H-pyrazol-1-yl)phenyl)dimethylphosphine oxide

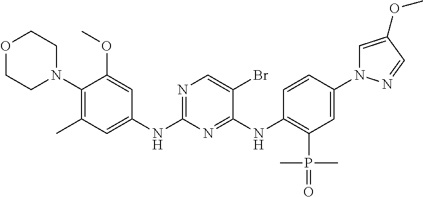 [SMILES: COc1cnn(-c2ccc(Nc3nc(Nc4cc(C)c(N5CCOCC5)c(OC)c4)ncc3Br)c(P(C)(C)=O)c2)c1]

### Step 1: Synthesis of Compound K-031-1

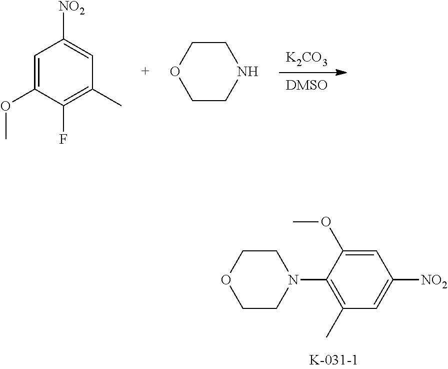

The compound K-031-1 was synthesized as the method described for compound K-018-8 using compound 2-fluoro-1-methoxy-3-methyl-5-nitrobenzene instead of raw compound K-018-6, compound morpholine instead of raw compound K-018-7.

For compound K-031-1, MS: 253.11 [M+H]+

### Step 2: Synthesis of Compound K-031-2

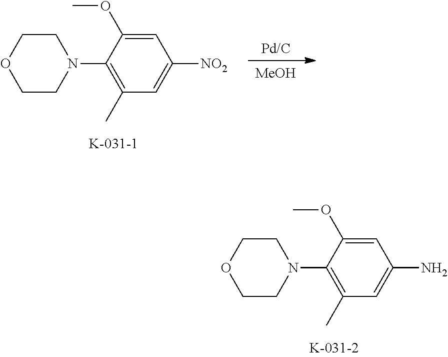 [SMILES: "MeOH" ; COc1cc([N+](=O)[O-])cc(C)c1N1CCOCC1 ; "Pd/C" ; COc1cc(N)cc(C)c1N1CCOCC1]

The compound K-031-2 was synthesized as the method described for compound K-018-9 using compound K-031-1 instead of raw compound K-018-8. For compound K-031-2, MS: 223.14 [M+H]+

### Step 3: Synthesis of Compound K-031

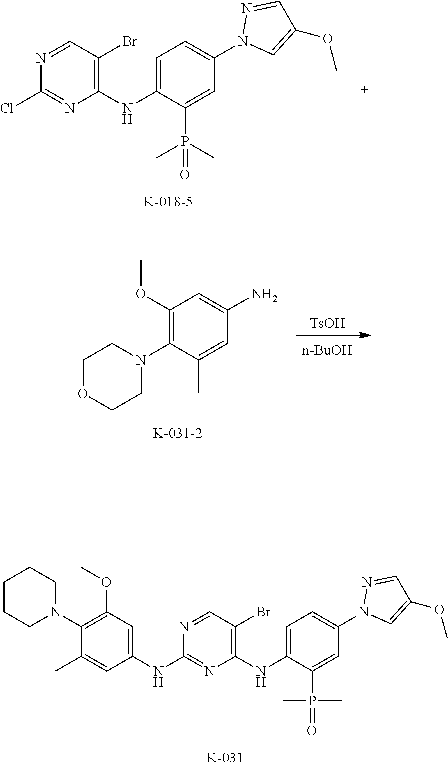

The compound K-031 was synthesized as the method described for compound K-018 using compound K-031-2 instead of raw compound K-018-9. For compound K-031, MS: 642.15 [M+H]+

### Example 10: Synthesis of Compound K-038

### (2-((5-Bromo-2-((4-(4-(dimethylamino)piperidin-1-yl)-5-methoxy-2-methylphenyl)amino)pyrimidine-4-yl)amino)-5-(4-methoxy-1H-pyrazol-1-yl)phenyl)dimethylphosphine oxide

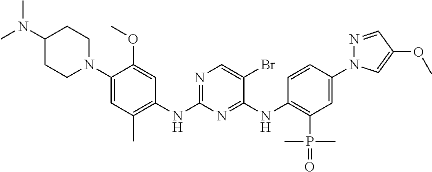

### Step 1: Synthesis of Compound K-038-3

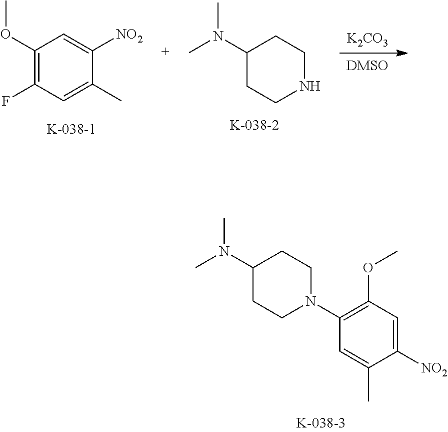

Dissolve compounds K-038-1 (490 mg) and K-038-2 (407 mg) in DMSO (10 mL), add K₂CO₃ (1.10 g) to the solution at room temperature: the reaction solution was heated to 90° C., and stirred for 16 hr. The reaction was complete as monitored by LCMS. The reaction solution was cooled down to room temperature, poured into water, and filtered to obtain a solid. The solid was purified with ether, and then dried to obtain compound K-038-3 (750 mg), MS: 294.17 [M+H]+

### Step 2: Synthesis of Compound K-038-4

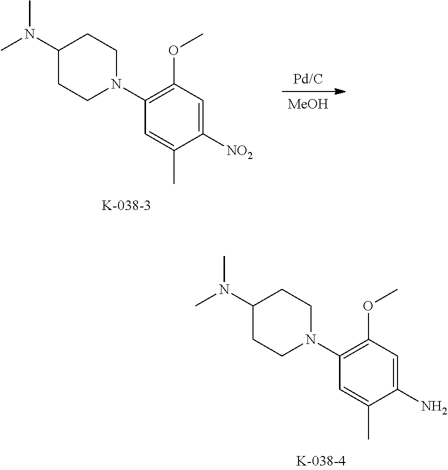 [SMILES: "MeOH" ; COc1cc([N+](=O)[O-])c(C)cc1N1CCC(N(C)C)CC1 ; "Pd/C" ; COc1cc(N)c(C)cc1N1CCC(N(C)C)CC1]

The compound K-038-4 was synthesized as the method described for compound K-018-9 using compound K-038-3 instead of raw compound K-018-8. For compound K-038-4, MS: 264.20 [M+H]+

### Step 3: Synthesis of Compound K-038

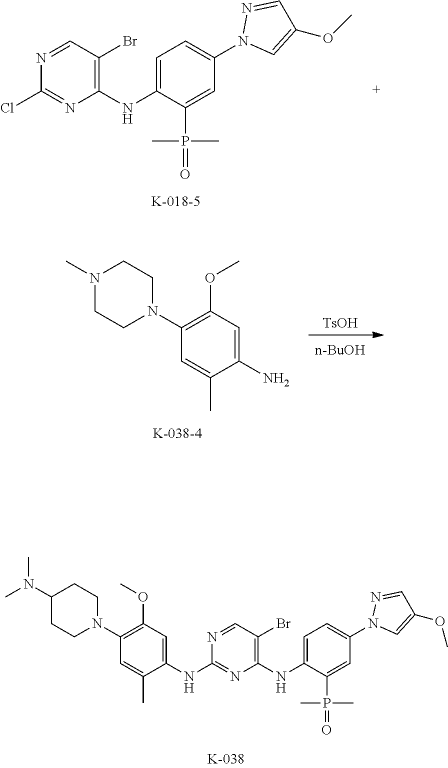

The compound K-038 was synthesized as the method described for compound K-018 using compound K-038-4 instead of raw compound K-018-9. For compound K-038, MS: 683.21 [M+H]+

### Example 11: Synthesis of Compound K-019

### (2-((5-Bromo-2-((5-methoxy-2-methyl-4-(4-(4-methylpiperazin-1-yl)piperidin-1-yl)phenyl)amino)pyrimidin-4-yl)amino)-5-(thiazol-2-yl)phenyl)dimethylphosphine oxide

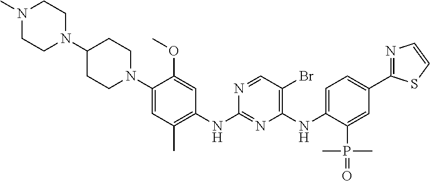 [SMILES: COc1cc(Nc2ncc(Br)c(Nc3ccc(-c4nccs4)cc3P(C)(C)=O)n2)c(C)cc1N1CCC(N2CCN(C)CC2)CC1]

### Step 1: Synthesis of Compound K-019-1

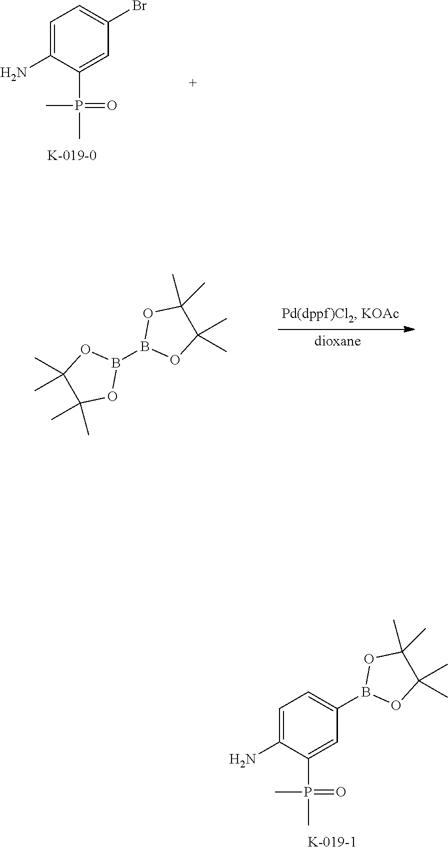

Add compound K-019-0 (5.0 g), Bis(pinacolato)diboron (7.68 g), Pd(dppf)Cl2 (1.65 g), potassium acetate (4.95 g), 1,4-Dioxane (50 mL) to the reaction flask, heat to 100° C. under N₂ protection, and stir with heating for 3 hr. The reaction was complete as monitored by LCMS, and then stopped. The reaction solution was cooled down to room temperature, and filtered; the solvent was removed to obtain the crude product. The crude product was separated and purified by column chromatography (dichloromethane:methanol=15:1), and then the solvent was removed to obtain the brown solid of desired product compound K-019-1 (2.0 g), MS: 296.15 [M+H]+

### Step 2: Synthesis of Compound K-019-2

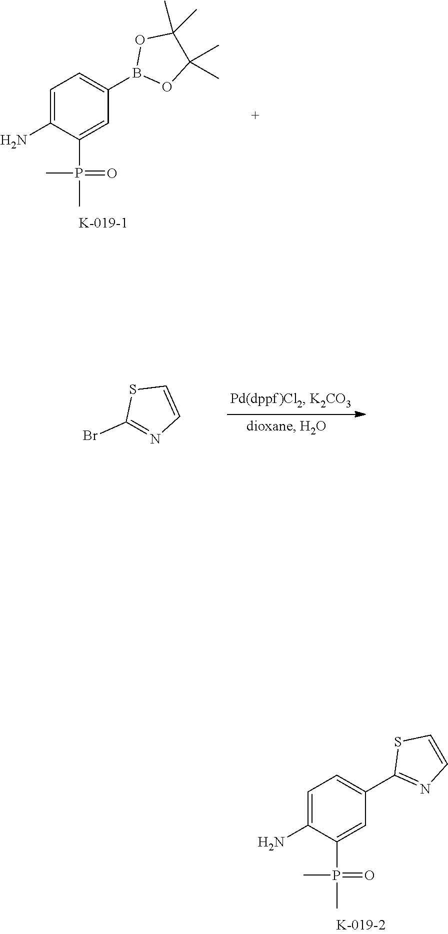 [SMILES: CC1(C)OB(c2ccc(N)c(P(C)(C)=O)c2)OC1(C)C ; Brc1nccs1 ; CP(C)(=O)c1cc(-c2nccs2)ccc1N]

Add compound K-019-1 (1.0 g), 2-Bromothiazole (590 mg), K₂CO₃ (1.67 g), Pd(dppf)Cl₂ (295 mg), 1,4-Dioxane (20 mL), and water (4 ml) to the reaction flask, heat to 100° C. under N₂ protection, and stir with heating for 12 hr. The reaction was complete as monitored by LCMS, and then stopped. Water (5 mL) was added to the reaction solution. And then the solution was extracted with dichloromethane (3×10 mL); the organic phase was washed with saturated brine (3×5 mL), dried over anhydrous sodium sulfate, separated and purified by column chromatography (dichloromethane:methanol=10:1); the solvent was removed to obtain the brown solid of desired product compound K-019-2 (900 mg), MS: 253.05 [M+H]+

### Step 3: Synthesis of Compound K-019-3

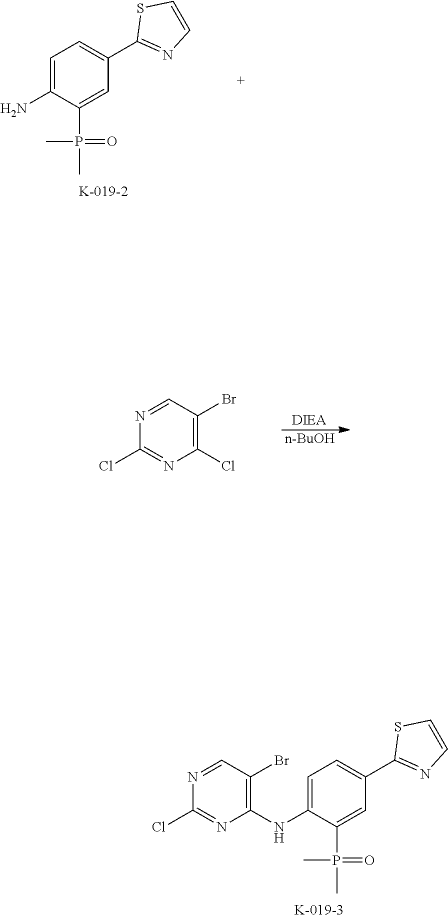

Add compound K-019-2 (2.13 g, 8.45 mmol), 5-Bromo-2,4-dichloropyrimidine (3.85 g, 16.91 mmol), DIEA (3.28 g, 25.36 mmol, 4.42 mL), n-BuOH (40 mL) to the reaction flask, heat to 120° C., and stir for 10 hr. The reaction was complete as monitored by LCMS. The reaction solution was cooled down to room temperature, and filtered, and then the filter cake was dried to obtain the yellowish solid K-019-3 (2.35 g, 5.31 mmol), MS: 442.94 [M+H]+

### Step 4: Synthesis of Compound K-019

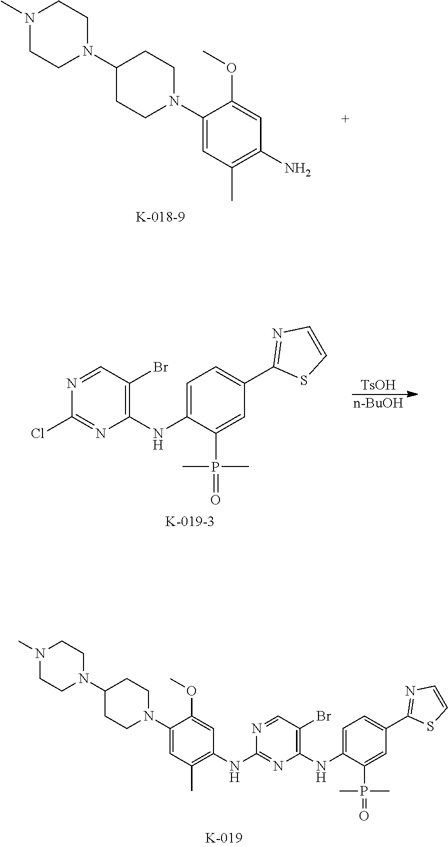

Add compounds K-019-3 (92.8 mg, 210.54 umol). K-018-9 (60.7 mg, 190.0 umol), p-toluenesulfonic acid (65.26 mg, 378.97 umol) and n-BuOH (1 ml) to the reaction flask, stir at 120° C. overnight. The reaction was complete as monitored by LCMS. The reaction solution was cooled down to room temperature, and spin-dried. Saturated Na₂CO₃ aqueous solution (10 mL) was added to the reaction solution. And then the solution was extracted with ethyl acetate (3×10 mL), dried over anhydrous sodium sulfate, and then filtered and spin-dried. The crude product was purified by PTLC (dichloromethane:methanol=10:1) to obtain the white solid of purified K-019 (15.0 mg, 11%), MS: 725.21 [M+H]+

¹H NMR (500 MHz, DMSO-d6) δ 11.29 (s, 1H), 8.79 (s, 1H), 8.45 (s, 1H), 8.19 (s, 1H), 7.96-7.94 (m, 1H), 7.90 (d, J=3.2 Hz, 1H), 7.81 (d, J=3.2 Hz, 1H), 7.61 (s, 1H), 6.87 (s, 1H), 6.78 (s, 1H), 3.68 (s, 3H), 3.48-3.42 (m, 2H), 3.32-3.25 (m, 1H), 2.78-2.52 (m, 7H), 2.39-2.14 (m, 6H), 2.08 (s, 3H), 1.92-1.75 (m, 8H), 1.68-1.52 (m, 2H).

### Example 12: Synthesis of Compound K-006

### (2-((5-Bromo-2-((5-ethyl-2-methoxy-4-(4-(4-methylpiperazin-1-yl)piperidin-1-yl)phenyl)amino)pyrimidin-4-yl)amino)-5-(2-(difluoromethyl)-2H-1,2,3-triazol-4-yl)phenyl)dimethylphosphine oxide

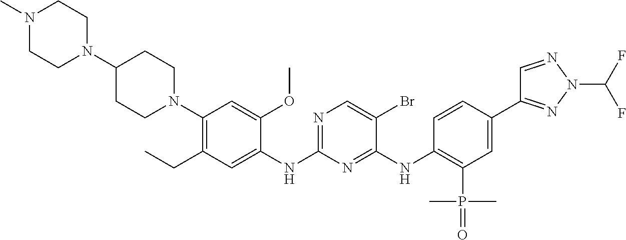 [SMILES: CCc1cc(Nc2ncc(Br)c(Nc3ccc(-c4cnn(C(F)F)n4)cc3P(C)(C)=O)n2)c(OC)cc1N1CCC(N2CCN(C)CC2)CC1]

### Step 1: Synthesis of Compound K-006-1

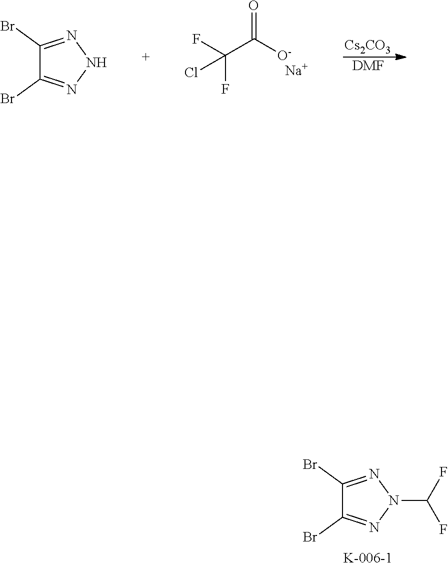

Add compound 4,5-Dibromo-2H-1,2,3-triazole (2.00 g), Sodium chlorodifluoroacetate (2.69 g), Cs₂CO₃ (8.62 g) and DMSO (20 mL) to the reaction flask, heat to 90° C., and stir with heating for 12 hr. The reaction was complete as monitored by LCMS, and then stopped. The reaction solution was poured into water (100 mL) solution, and extracted with dichloromethane (3×40 mL); the organic phase was washed with saturated brine (3×30 mL), dried over anhydrous sodium sulfate, separated and purified by column chromatography (n-hexane:EA=10:1) to obtain the white solid of desired product compound K-006-1 (4.20 g), MS: 275.85 [M+H]⁺

### Step 2: Synthesis of Compound K-006-2

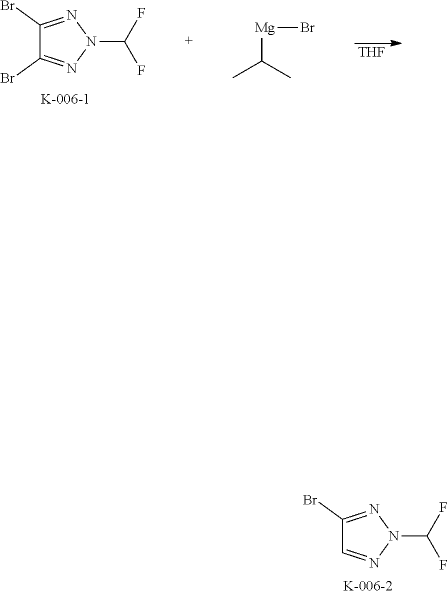

K-006-1 (1.0 g, 3.61 mmol) was dissolved in THF (10 mL), the solution was cooled down to −10° C., and isopropylmagnesium bromide (1.3 M, 3.06 mL) was slowly added dropwise. Keep the reaction temperature at −5 to 5° C., and continue stirring for 30 min, then slowly add water (2 ml) dropwise and stir for 1 hr. Add hydrochloric acid to adjust pH value of 1-2; the reaction solution was extracted with ethyl acetate (3×30 mi); the organic phase was washed with saturated brine (3×30 mL), and dried over anhydrous sodium sulfate to obtain a brown liquid of desired product compound K-006-2 (500 mg), MS: 197.94 [M+H]+

### Step 3: Synthesis of Compound K-006-3

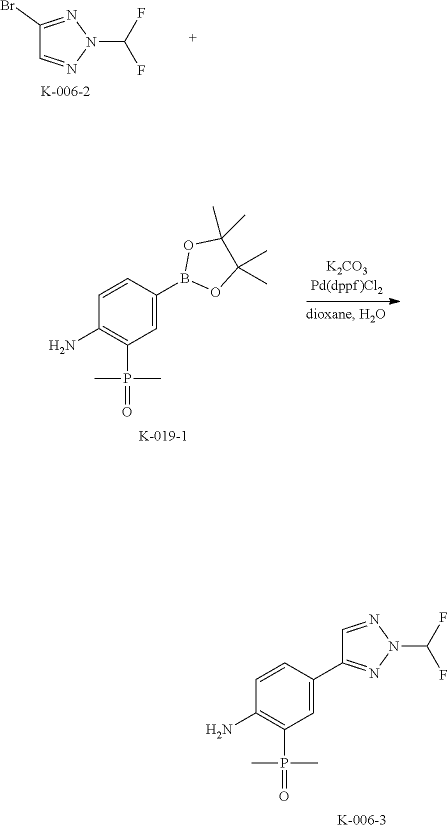 [SMILES: FC(F)n1ncc(Br)n1 ; CC1(C)OB(c2ccc(N)c(P(C)(C)=O)c2)OC1(C)C ; CP(C)(=O)c1cc(-c2cnn(C(F)F)n2)ccc1N]

The compound K-006-3 was synthesized as the method described for compound K-019-2 using compound K-006-2 instead of raw compound 2-Bromothiazole. For compound K-006-3, MS: 287.08 [M+H]⁺

### Step 4: Synthesis of Compound K-006-4

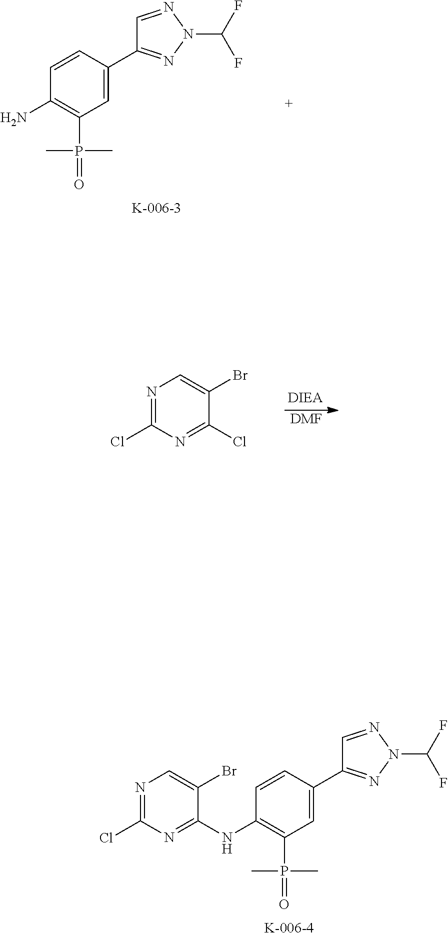

The compound K-006-4 was synthesized as the method described for compound K-018-5 using compound K-006-3 instead of raw compound K-018-4. For compound K-006-4, MS: 476.97 [M+H]⁺

### Step 5: Synthesis of Compound K-006

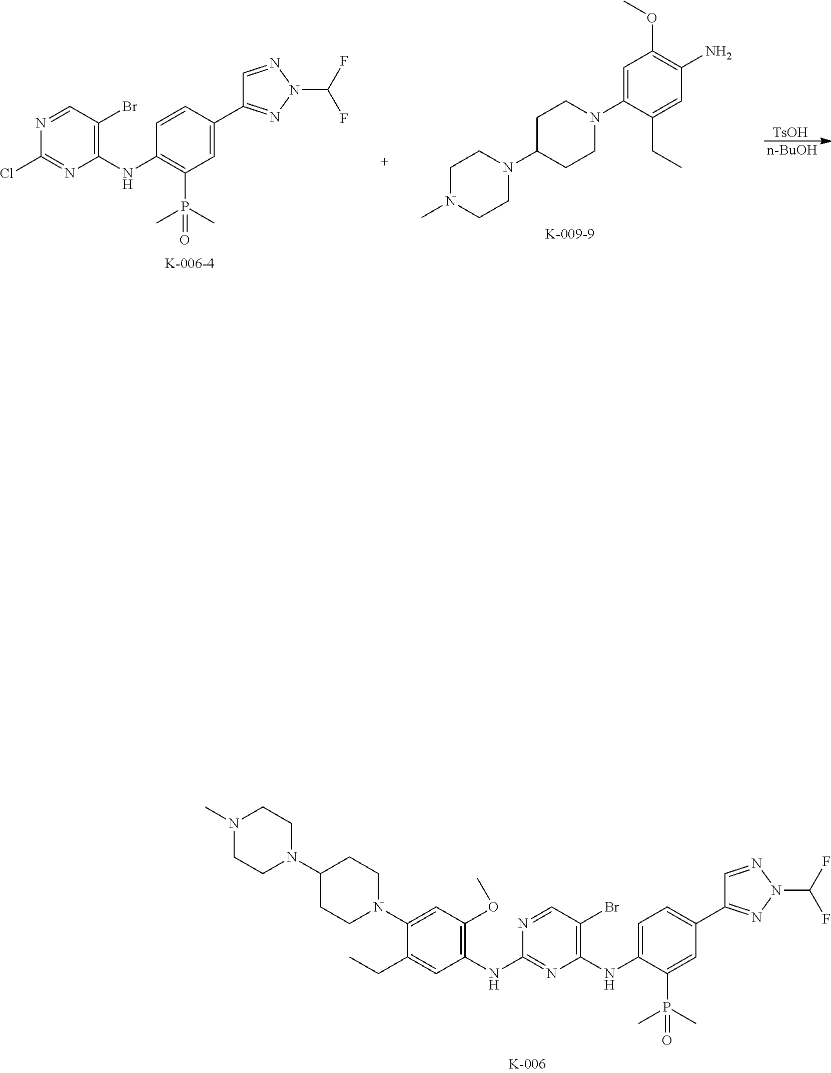

The compound K-006 was synthesized as the method described for compound K-018 using compound K-009-9 instead of raw compound K-018-9, compound K-006-4 instead of raw compound K-018-5. For compound K-006, MS: 773.25 [M+H]⁺

### Example 13: Synthesis of Compound K-024

### (2-((5-Bromo-2-((5-methoxy-2-methyl-4-morpholinophenyl)amino)pyrimidin-4-yl)amino)-5-(thiazol-2-yl)phenyl)dimethylphosphine oxide

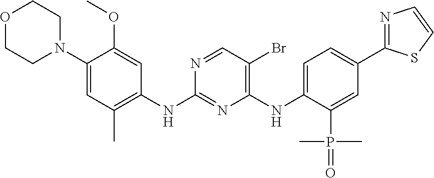 [SMILES: COc1cc(Nc2ncc(Br)c(Nc3ccc(-c4nccs4)cc3P(C)(C)=O)n2)c(C)cc1N1CCOCC1]

### Step 1: Synthesis of Compound K-024-1

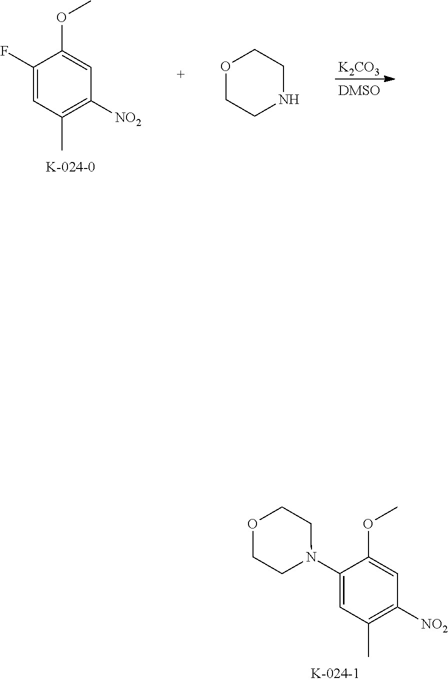

Add compounds K-024-0 (1.00 g), morpholine (611 mg), K₂CO₃ (2.22 g) and DMSO (10 ml) to the reaction flask, heat to 90° C., and stir with heating for 12 hr. The reaction was complete as monitored by LCMS, and then stopped. The reaction solution was poured into water (30 mL), and filtered with suction; the filter cake was washed with water and dried to obtain the yellow solid of desired product compound K-024-1 (1.1 g), MS: 253.11 [M+H]⁺

### Step 2: Synthesis of Compound K-024-2

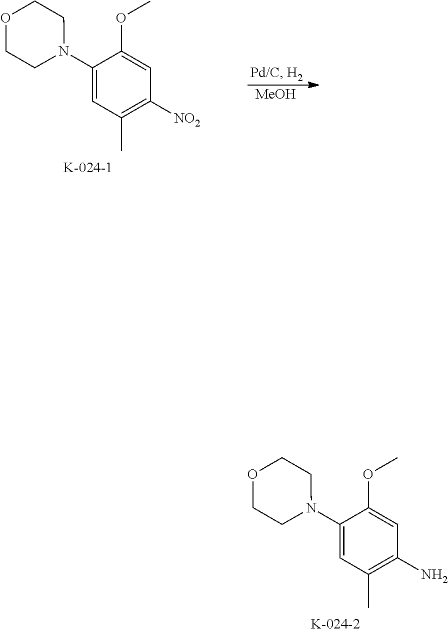 [SMILES: "MeOH" ; COc1cc([N+](=O)[O-])c(C)cc1N1CCOCC1 ; "Pd/C, H2" ; COc1cc(N)c(C)cc1N1CCOCC1]

Add compound K-024-1 (1.1 g), Pd/C (300 mg, 10%) and methanol (20 mL) to the reaction flask, introduce H₂; the reaction solution was stirred at room temperature for 3 hr. The reaction was complete as monitored by LCMS, and then stopped. The solution was filtered with suction, and rinsed with methanol (20 mL); collect the organic phase, remove the solvent, and obtain the white solid of desired product compound K-024-2, MS: 223.14 [M+H]⁺

### Step 3: Synthesis of Compound K-024

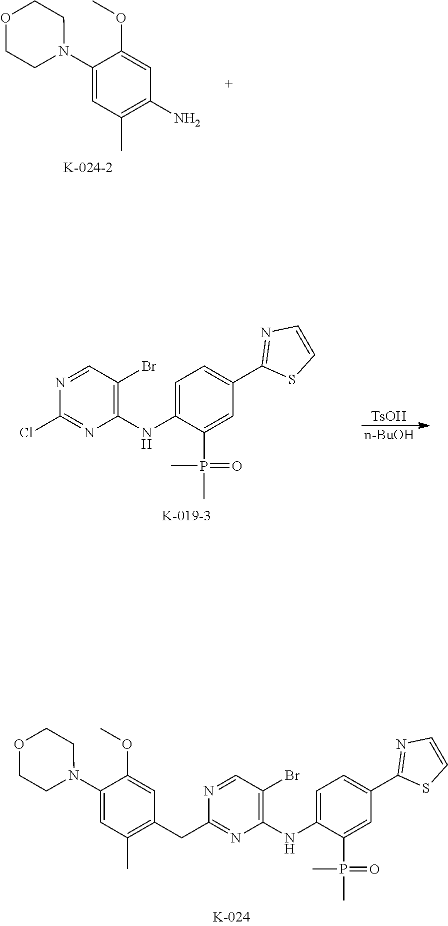

The compound K-024 was synthesized as the method described for compound K-019 using compound K-024-2 instead of raw compound K-018-9. For compound K-024, MS: 629.10 [M+H]⁺

¹H NMR (500 MHz, DMSO-d6) δ 11.22 (s, 1H), 8.49 (s, 1H), 8.42 (s, 1H), 8.27 (s, 1H), 8.07-8.02 (m, 1H), 7.92 (d, J=3.2 Hz, 2H), 7.89 (d, J=9.3 Hz, 1H), 7.81 (d, J=3.2 Hz, 111), 6.80 (s, 1H), 3.89 (s, 311), 3.83-3.76 (m, 4H), 3.23-3.13 (m, 4H), 1.87 (s, 311), 1.84 (s, 311).

### Example 14: Synthesis of Compound K-027

### 5-((5-Bromo-4-((2-(dimethylphosphine oxide)-4-(thiazol-2-yl)phenyl)amino)pyrimidin-2-yl)amino)-4-methoxy-2-morpholinobenzonitrile

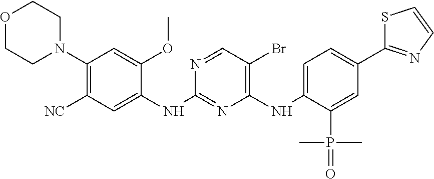 [SMILES: COc1cc(N2CCOCC2)c(C#N)cc1Nc1ncc(Br)c(Nc2ccc(-c3nccs3)cc2P(C)(C)=O)n1]

### Step 1: Synthesis of Compound K-027-1

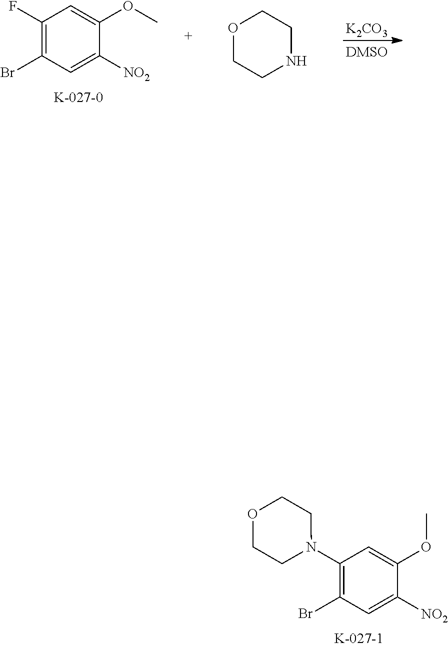

Add compound K-027-0 (3.00 g), morpholine (4.14 g), K₂CO₃ (6.24 g) and DMF (30 mL) to the reaction flask, heat to 100° C. and stir with heating for 12 hr. The reaction was complete as monitored by LCMS, and then stopped. The reaction solution was poured into water (25 mL), and filtered with suction; the filter cake was washed with water and dried to obtain the yellow solid of desired product compound K-027-1 (4.20 g), MS: 317.01 [M+H]⁺

### Step 2: Synthesis of Compound K-027-2

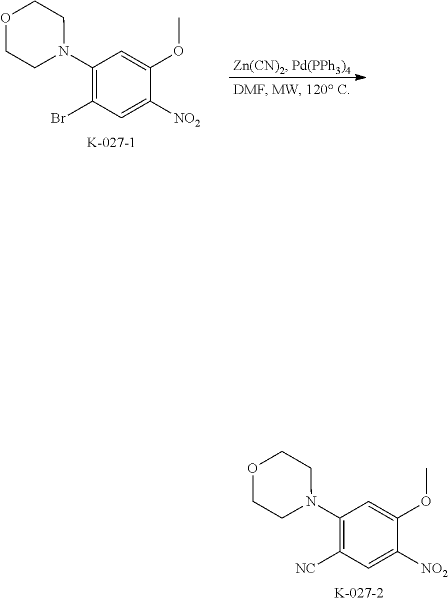

Dissolve K-027-1 (1.1 g, 3.47 mmol), Zn(CN)₂ (611 mg, 5.20 mmol), Pd(PPh₃)₄ (400 mg, 0.34 mmol) in DMF (10 mL). Under nitrogen protection, keep microwave reaction at 120° C. for 2 hr. The reaction was complete as monitored by LCMS. The reaction solution was cooled down to room temperature, and add water (20 mL) into the solution. And then the reaction solution was filtered, rinsed with a small amount of water, then washed with dichloromethane (15 mL), dried over anhydrous sodium sulfate, then filtered and spin-dried, and purified by column chromatography (dichloromethane/methanol=15:1). And then the solvent was spin-dried to obtain the white solid of desired product compound K-027-2 (700 mg, 2.66, 77%), MS: 264.09 [M+H]⁺

### Step 3: Synthesis of Compound K-027-3

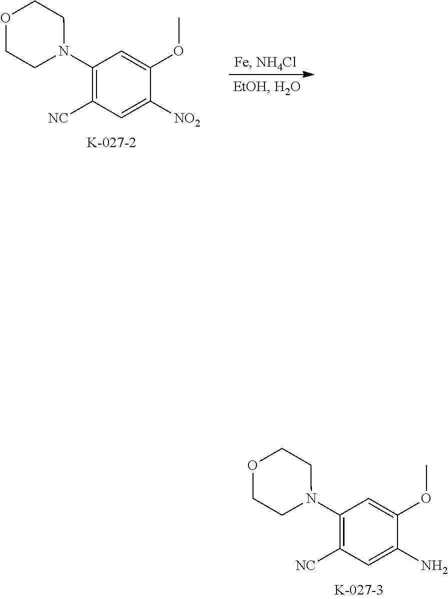

Dissolve K-027-2 (700 mg) in ethanol (5 mL) and water (2 mL). Add reduced iron powder (1.5 g) and NH₄Cl (1.5 g) to the solution, and keep the reaction at 80° C. for 2 hr. The reaction was complete as monitored by LCMS. The reaction solution was cooled down to room temperature; 10 mL of saturated Na₂CO₃ aqueous solution was added to the solution. And then the reaction solution was extracted with dichloromethane (3×10 mL), dried, filtered, and spin-dried to obtain the yellowish solid K-027-3 (500 mg), MS: 234.12 [M+H]⁺

### Step 4: Synthesis of Compound K-027

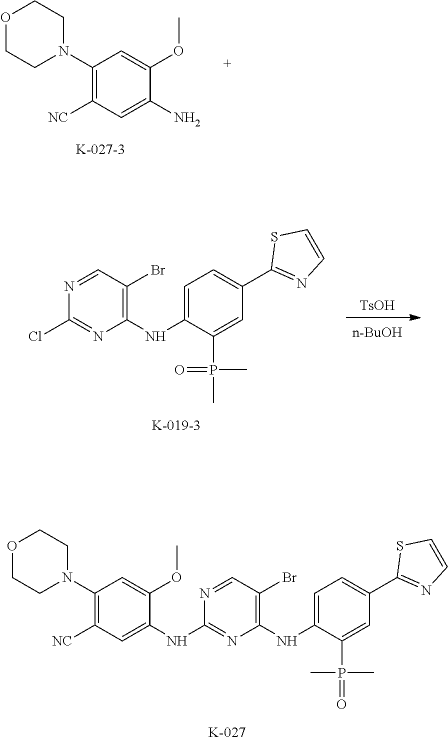

The compound K-027 was synthesized as the method described for compound K-019 using compound K-027-3 instead of raw compound K-018-9. For compound K-027, MS: 640.08 [M+H]⁺

### Example 15: Synthesis of Compound K-003

### (2-((5-Bromo-2-((2-methoxy-4-(4-(4-methylpiperazin-1-yl)piperidin-1-yl)-5-(tetrahydro-2H-pyran-4-yl)phenyl)amino)pyrimidin-4-yl)amino)-5-(thiazol-4-yl)phenyl)dimethylphosphine oxide

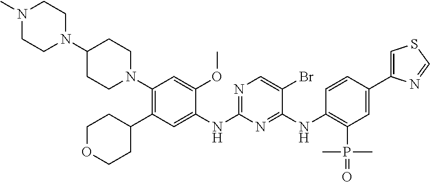 [SMILES: COc1cc(N2CCC(N3CCN(C)CC3)CC2)c(C2CCOCC2)cc1Nc1ncc(Br)c(Nc2ccc(-c3cscn3)cc2P(C)(C)=O)n1]

### Step 1: Synthesis of Compound K-003-1

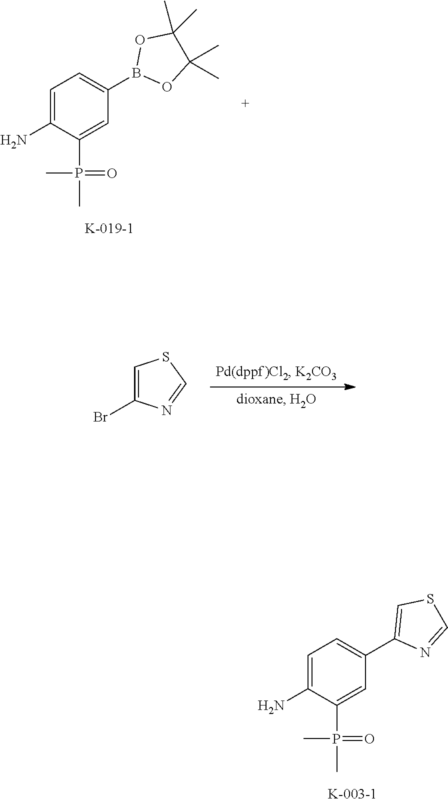 [SMILES: CC1(C)OB(c2ccc(N)c(P(C)(C)=O)c2)OC1(C)C ; Brc1cscn1 ; "Pd(dppf)Cl2, K2CO3" ; "dioxane, H2O" ; CP(C)(=O)c1cc(-c2cscn2)ccc1N]

The compound K-003-1 was synthesized as the method described for compound K-019-2 using 4-Bromothiazole instead of 2-Bromothiazole. For compound K-003-1, MS: 253.05 [M+H]⁺

### Step 2: Synthesis of Compound K-003-2

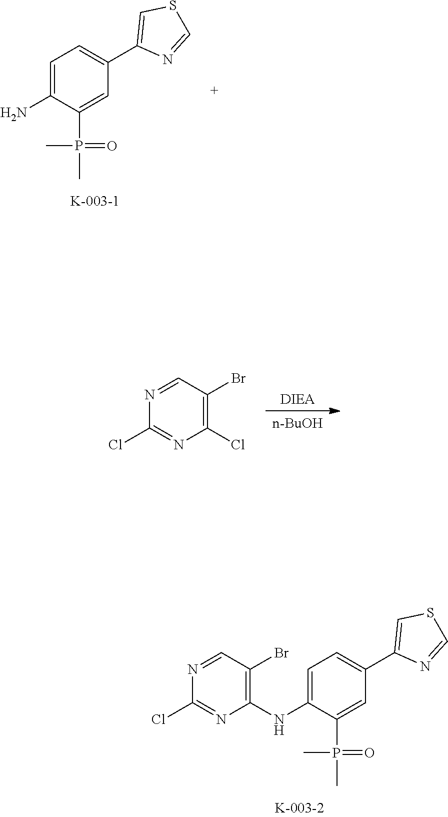

The compound K-003-2 was synthesized as the method described for compound K-019-3 using compound K-003-1 instead of compound K-019-2. For compound K-003-2, MS: 442.94 [M+H]⁺

### Step 3: Synthesis of Compound K-003-3

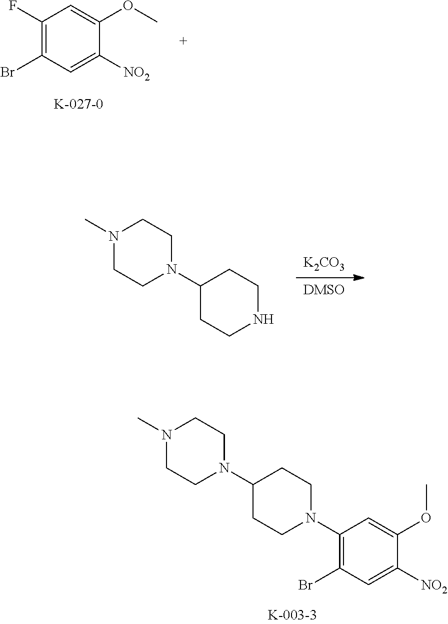

The compound K-003-3 was synthesized as the method described for compound K-024-1 using compound K-027-0 instead of raw compound K-024-0, compound 1-Methyl-4-(piperidin-4-yl)piperazine instead of morpholine. For compound K-003-3, MS: 413.11 [M+H]⁺

### Step 4: Synthesis of Compound K-003-4

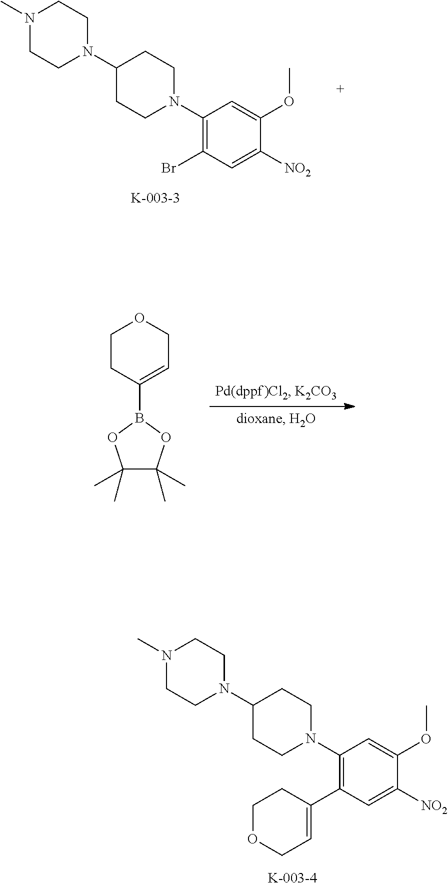

The compound K-003-4 was synthesized as the method described for compound K-019-2 using 3,6-Dihydro-2H-pyran-4-boronic acid pinacol ester instead of 2-Bromothiazole, compound K-003-3 instead of compound K-019-1. For compound K-003-4, MS: 417.24 [M+H]⁺

### Step 5: Synthesis of Compound K-003-5

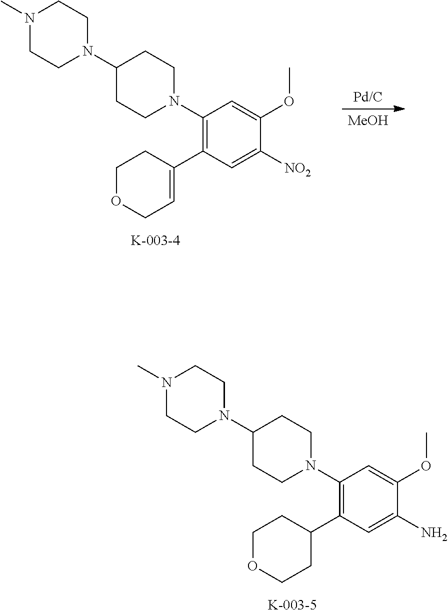

The compound K-003-5 was synthesized as the method described for compound K-024-2 using compound K-003-4 instead of raw compound K-024-1. For compound K-003-5, MS: 389.28 [M+H]⁺

### Step 6: Synthesis of Compound K-003

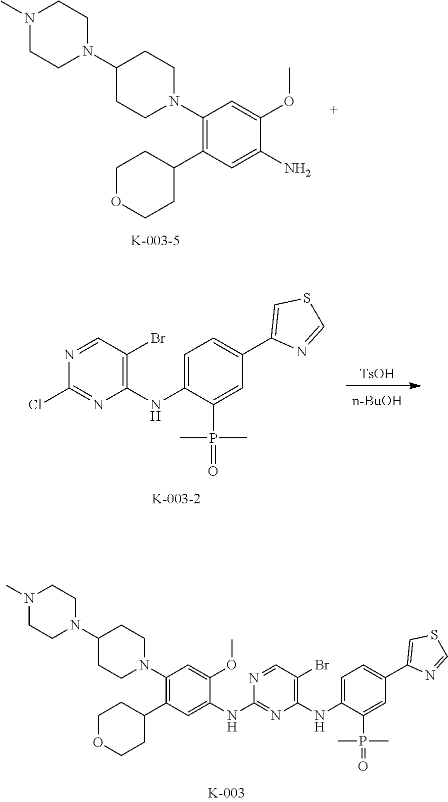

Add compounds K-003-2 (92.8 mg, 210.54 umol), K-003-5 (73.0 mg, 190.0 umol), p-toluenesulfonic acid (65.0 mg, 378.0 umol) and n-BuOH (1 mL) to the reaction flask, stir at 120° C. overnight. The reaction was complete as monitored by LCMS. The reaction solution was cooled down to room temperature, and spin-dried. Saturated Na₂CO₃ aqueous solution (10 mL) was added to the reaction solution. And then the solution was extracted with dichloromethane (3×10 mL), dried over anhydrous sodium sulfate, and then filtered and spin-dried. The crude product was purified by PTLC (dichloromethane:methanol=10:1) to obtain desired product compound K-003, MS: 795.25 [M+H]⁺

### Example 16: Synthesis of Compound K-029

### (2-((5-Bromo-2-((2-methoxy-5-methyl-4-(4-methylpiperazin-1-yl)phenyl)amino)pyrimidin-4-yl)amino)-5-(thiazol-2-yl)phenyl)dimethylphosphine oxide

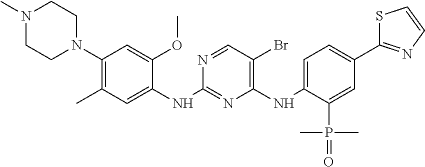 [SMILES: COc1cc(N2CCN(C)CC2)c(C)cc1Nc1ncc(Br)c(Nc2ccc(-c3nccs3)cc2P(C)(C)=O)n1]

### Step 1: Synthesis of Compound K-029-1

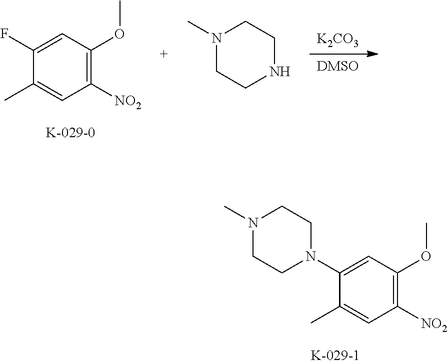

The compound K-029-1 was synthesized as the method described for compound K-024-1 using compound K-029-0 instead of raw compound K-024-0. N-methylpiperazine instead of morpholine. For compound K-029-1, MS: 266.14 [M+H]⁺

### Step 2: Synthesis of Compound K-029-2

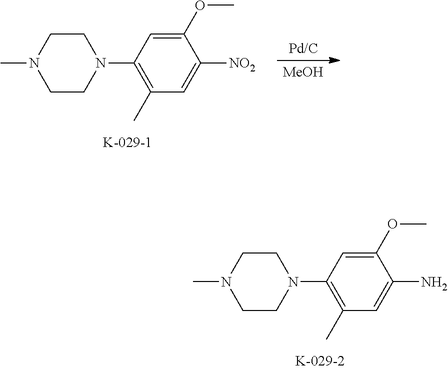

The compound K-029-2 was synthesized as the method described for compound K-024-2 using compound K-029-1 instead of raw compound K-024-1. For compound K-029-2, MS: 236.17 [M+H]⁺

### Step 3: Synthesis of Compound K-029

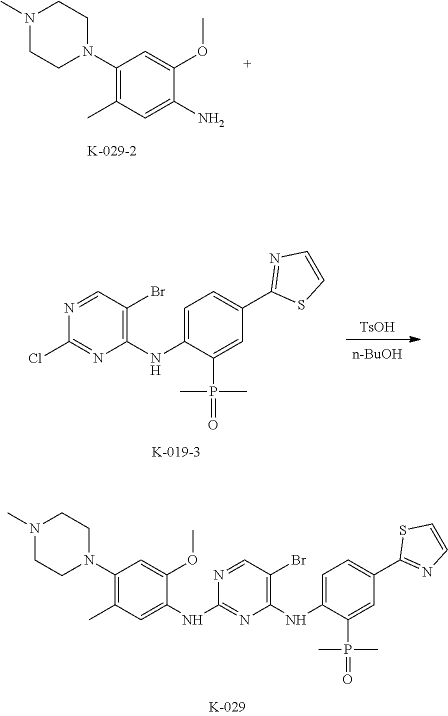

The compound K-029 was synthesized as the method described for compound K-019 using compound K-029-2 instead of raw compound K-018-9. For compound K-029, MS: 642.13 [M+H]⁺

### Example 17: Synthesis of Compound K-030

### (2-((5-Bromo-2-((5-methoxy-2-methyl-4-morpholinophenyl)amino)pyrimidin-4-yl)amino)-5-(1-methyl-1H-pyrazol-4-yl)phenyl)dimethylphosphine oxide

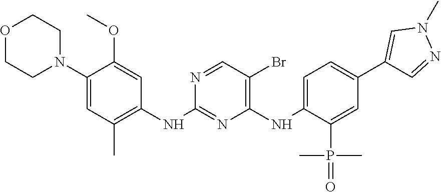 [SMILES: COc1cc(Nc2ncc(Br)c(Nc3ccc(-c4cnn(C)c4)cc3P(C)(C)=O)n2)c(C)cc1N1CCOCC1]

### Step 1: Synthesis of Compound K-030-1

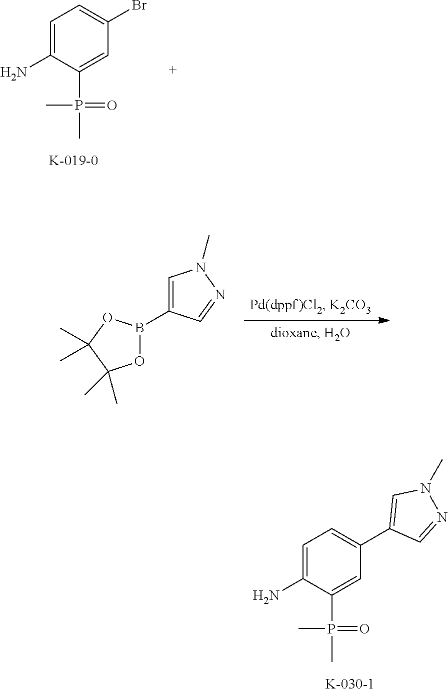 [SMILES: CP(C)(=O)c1cc(Br)ccc1N ; Cn1cc(B2OC(C)(C)C(C)(C)O2)cn1 ; "Pd(dppf)Cl2, K2CO3" ; "dioxane, H2O" ; Cn1cc(-c2ccc(N)c(P(C)(C)=O)c2)cn1]

The compound K-030-1 was synthesized as the method described for compound K-019-1 using 1-Methylpyrazole-4-boronic acid pinacol ester instead of Bis(pinacolato)diboron. For compound K-030-1, MS: 236.17 [M+H]⁺

### Step 2: Synthesis of Compound K-030-2

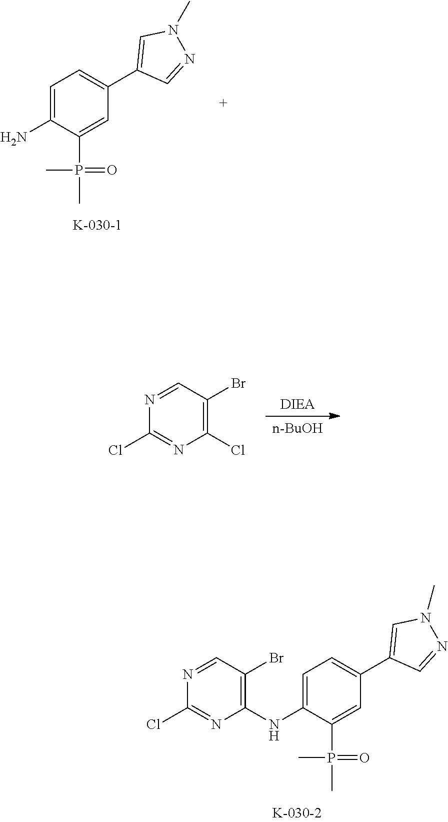

The compound K-030-2 was synthesized as the method described for compound K-019-3 using compound K-030-1 instead of compound K-019-2. For compound K-030-2, MS: 440.00 [M+H]⁺

### Step 3: Synthesis of Compound K-030

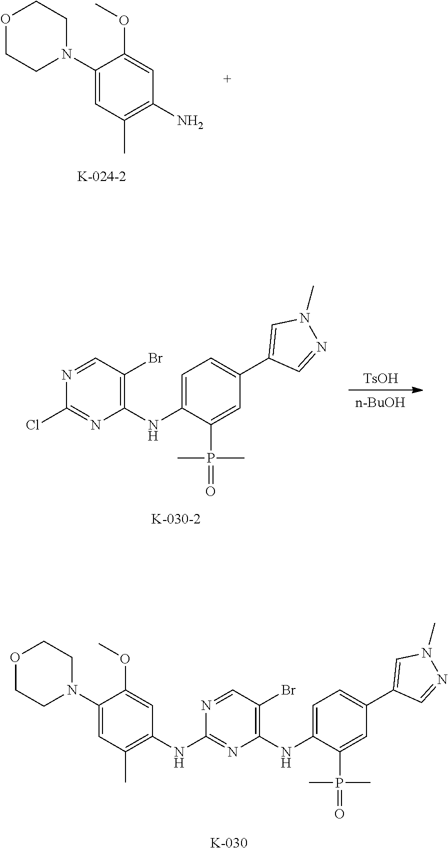

The compound K-030 was synthesized as the method described for compound K-019 using compound K-024-2 instead of raw compound K-018-9, compound K-030-2 instead of compound K-019-3. For compound K-030, MS: 626.16 [M+H]⁺

### Example 18: Synthesis of Compound K-048

### (3-((5-Bromo-2-((5-ethyl-2-methoxy-4-(4-morpholinopiperidin-1-yl)phenyl)amino)pyrimidin-4-yl)amino)-2′-fluoro-[1,1′-biphenyl]-3-yl)dimethylphosphine oxide

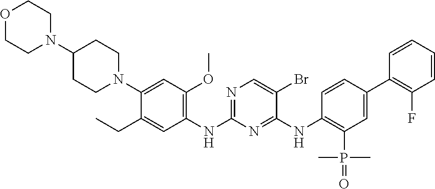 [SMILES: CCc1cc(Nc2ncc(Br)c(Nc3ccc(-c4ccccc4F)cc3P(C)(C)=O)n2)c(OC)cc1N1CCC(N2CCOCC2)CC1]

### Step 1: Synthesis of Compound K-048-1

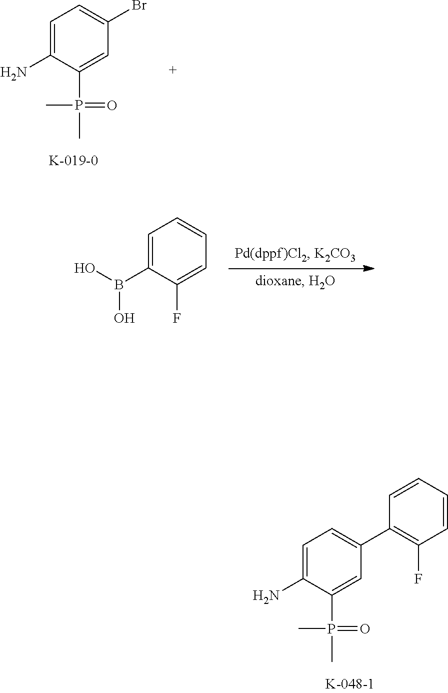 [SMILES: CP(C)(=O)c1cc(Br)ccc1N ; OB(O)c1ccccc1F ; "Pd(dppf)Cl2, K2CO3" ; "dioxane, H2O" ; CP(C)(=O)c1cc(-c2ccccc2F)ccc1N]

The compound K-048-1 was synthesized as the method described for compound K-019-1 using 2-Fluorophenylboronic acid instead of Bis(pinacolato)diboron. For compound K-048-1, MS: 264.09 [M+H]⁺

### Step 2: Synthesis of Compound K-048-2

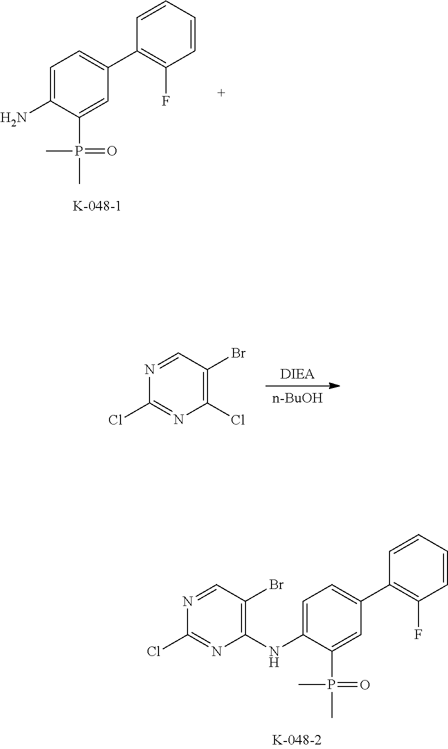

The compound K-048-2 was synthesized as the method described for compound K-019-3 using compound K-048-1 instead of compound K-019-2. For compound K-048-2, MS: 453.98 [M+H]⁺

### Step 3: Synthesis of Compound K-048-3

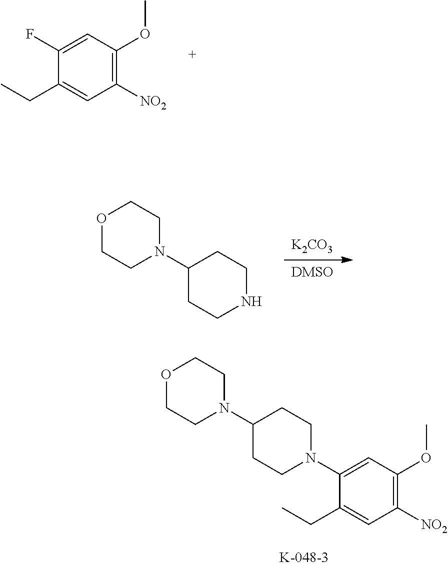

The compound K-048-3 was synthesized as the method described for compound K-024-1 using 4-Morpholinopiperidine and 1-Ethyl-2-fluoro-4-methoxy-5-nitrobenzene instead of two raw compounds. For compound K-048-3, MS: 350.20 [M+H]⁺

### Step 4: Synthesis of Compound K-048-4

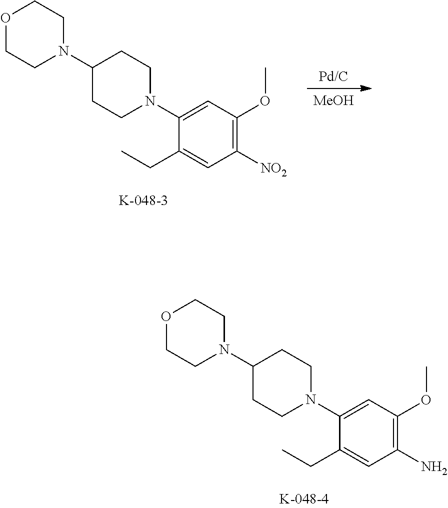 [SMILES: "MeOH" ; CCc1cc([N+](=O)[O-])c(OC)cc1N1CCC(N2CCOCC2)CC1 ; "Pd/C" ; CCc1cc(N)c(OC)cc1N1CCC(N2CCOCC2)CC1]

The compound K-048-4 was synthesized as the method described for compound K-024-2 using compound K-048-3 instead of raw compound K-024-1. For compound K-048-4, MS: 320.23 [M+H]⁺

### Step 5: Synthesis of Compound K-048

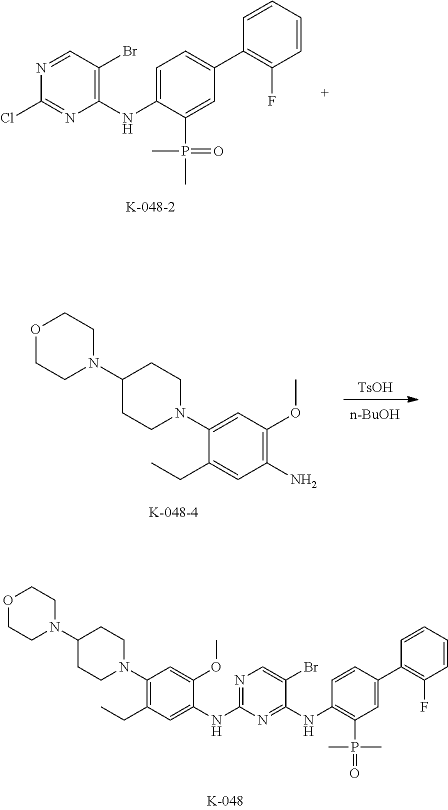

The compound K-048 was synthesized as the method described for compound K-019 using compound K-048-4 instead of raw compound K-018-9, compound K-048-2 instead of compound K-019-3. For compound K-048, MS: 737.23 [M+H]⁺

### Example 19: Synthesis of Compound K-044

### (2-((5-Bromo-2-((4-(4-(dimethylamino)piperidin-1-yl)-5-ethyl-2-methoxyphenyl)amino)pyrimidin-4-yl)amino)-5-(1-methyl-1H-pyrazol-4-yl)phenyl)dimethylphosphine oxide

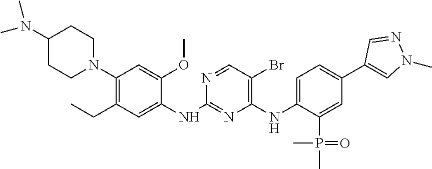

### Step 1: Synthesis of Compound K-044-1

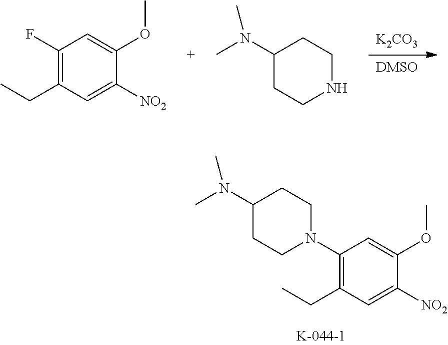

The compound K-044-1 was synthesized as the method described for compound K-018-8 using compound 1-Ethyl-2-fluoro-4-methoxy-5-nitrobenzene instead of raw compound K-018-6, compound 4-Dimethylaminopiperidine instead of raw compound K-018-7. For compound K-044-1, MS: 308.19 [M+H]⁺

### Step 2: Synthesis of Compound K-044-2

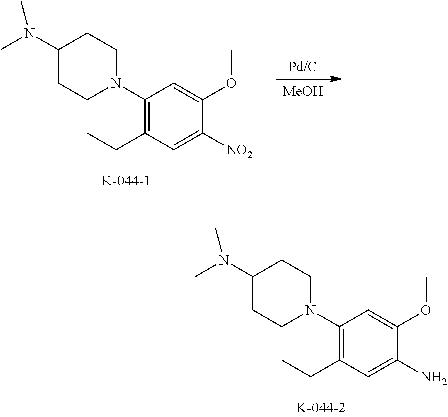

The compound K-044-2 was synthesized as the method described for compound K-018-9 using compound K-044-1 instead of raw compound K-018-8. For compound K-044-2, MS: 278.22 [M+H]⁺

### Step 3: Synthesis of Compound K-044

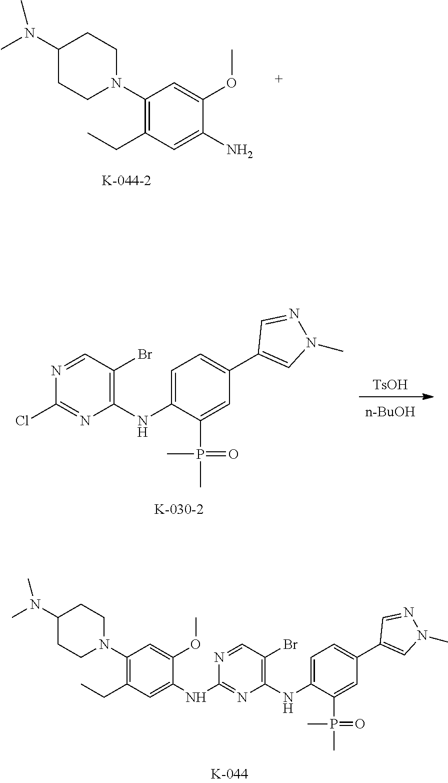

The compound K-044 was synthesized as the method described for compound K-019 using compound K-044-2 instead of raw compound K-018-9, compound K-030-2 instead of compound K-019-3. For compound K-044, MS: 681.24 [M+H]⁺

### Example 20: Synthesis of Compound K-042

### (2-((5-Bromo-2-((5-ethyl-2-methoxy-4-(4-morpholinopiperidin-1-vi)phenyl)amino)pyrimidin-4-yl)amino)-5-(1-methyl-1-pyrazol-4-yl)phenyl)-dimethylphosphine oxide

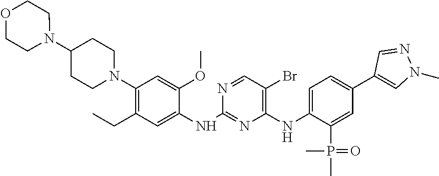 [SMILES: CCc1cc(Nc2ncc(Br)c(Nc3ccc(-c4cnn(C)c4)cc3P(C)(C)=O)n2)c(OC)cc1N1CCC(N2CCOCC2)CC1]

### Step 1: Synthesis of Compound K-042

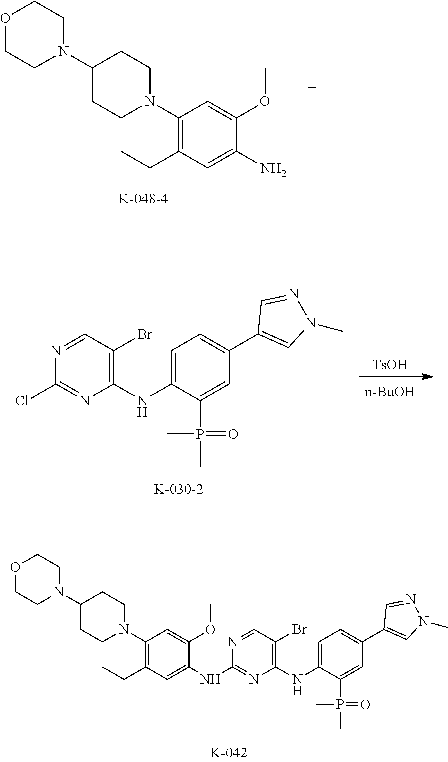

The compound K-042 was synthesized as the method described for compound K-019 using compound K-048-4 instead of raw compound K-018-9, compound K-030-2 instead of compound K-019-3. For compound K-042, MS: 723.25 [M+H]⁺

### Example 21: Synthesis of Compound K-047

### (4-((5-Bromo-2-((5-ethyl-2-methoxy-4-(4-morpholinopiperidin-1-yl)phenyl)amino)pyrimidin-4-yl)amino)-[1,1′-biphenyl]-3-yl)dimethylphosphine oxide

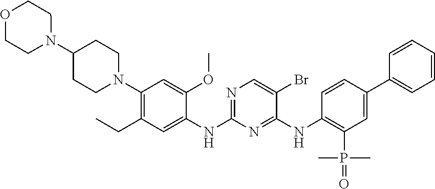 [SMILES: CCc1cc(Nc2ncc(Br)c(Nc3ccc(-c4ccccc4)cc3P(C)(C)=O)n2)c(OC)cc1N1CCC(N2CCOCC2)CC1]

### Step 1: Synthesis of Compound K-047-1

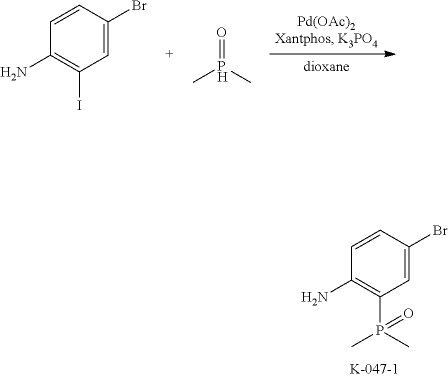

Add 4-Bromo-2-iodoaniline (3.0 g), dimethylphosphine oxide (865 mg), K₃PO₄ (6.41 g), Pd(OAc)₂ (226 mg). Xantphos (1.75 g), Dioxane (60 mL) into the reaction flask, heat to 90° C., and stir with heating for 4 hr under nitrogen protection. The reaction was complete as monitored by LCMS, and then stopped. Add water (50 mL) into the reaction solution. And then the reaction solution was extracted with dichloromethane (3×50 mL); the organic phase was washed with saturated brine (3×30 ml), dried over anhydrous sodium sulfate, separated and purified by column chromatography (dichloromethane:methanol=15:1), and the solvent was removed to obtain the brown solid of desired product compound K-047-1 (2.0 g), MS: 247.98 [M+H]⁺

### Step 2: Synthesis of Compound K-047-2

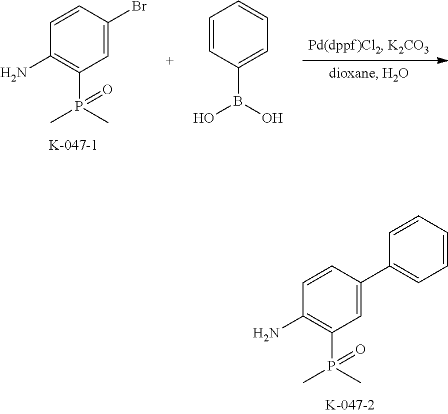 [SMILES: CP(C)(=O)c1cc(Br)ccc1N ; OB(O)c1ccccc1 ; "Pd(dppf)Cl2, K2CO3" ; "dioxane, H2O" ; CP(C)(=O)c1cc(-c2ccccc2)ccc1N]

Add compound K-047-1 (1.0 g), phenylboronic acid (590 mg), K₂CO₃ (1.67 g), Pd(dppf)Cl₂ (295 mg), dioxane (20 mL) and water (4 mL) into the reaction flask, heat to 100° C., and stir with heating for 12 hr under nitrogen protection. The reaction was complete as monitored by LCMS, and then stopped. Water (50 mL) was added to the reaction solution. And then the reaction solution was extracted with dichloromethane (3×50 mL); the organic phase was washed with saturated brine (3×30 mL), dried over anhydrous sodium sulfate, separated and purified by column chromatography (dichloromethane:methanol=10:1), and the solvent was removed to obtain the brown solid of desired product compound K-047-2 (900 mg), MS: 246.10 [M+H]⁺

### Step 3: Synthesis of Compound K-047-3

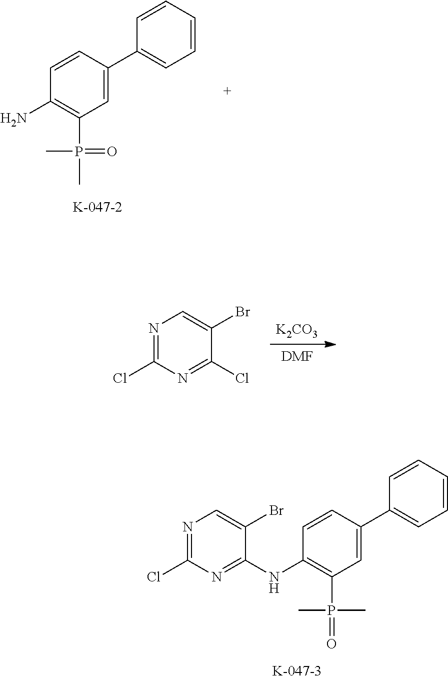

Add compound K-047-2 (1.0 g), 5-Bromo-2,4-dichloropyrimidine (1.39 g), K₂CO₃ (1.69 g) and DMF (20 mL) into the reaction flask, heat to 100° C., and stir with heating for 12 hr. The reaction was complete as monitored by LCMS, and then stopped. The reaction solution was poured into water (50 mL), and extracted with ethyl acetate (3×50 mL); the organic phase was washed with saturated brine (3×30 mL), dried over anhydrous sodium sulfate, separated and purified by column chromatography (dichloromethane:methanol=13:1), and the solvent was removed to obtain the yellow solid of desired product compound K-047-3 (700 mg), MS: 435.99 [M+H]⁺

### Step 4: Synthesis of Compound K-047

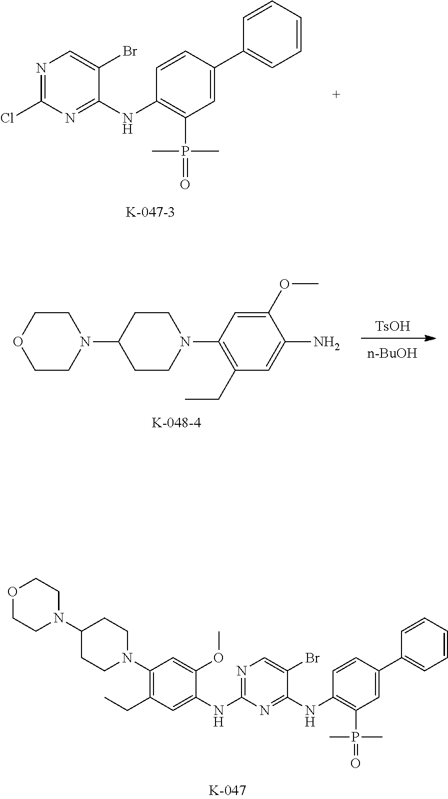

Add compound K-047-3 (100 mg), K-048-4 (76 mg), p-toluenesulfonic acid (60 mg) and n-butanol (2 mL) into the reaction flask, heat to 120° C., and stir for 12 hr. The reaction was complete as monitored by LCMS, and then stopped. The reaction solution was poured into sodium carbonate solution (5 mL), and extracted with dichloromethane (3×50 mL); the organic phase was washed with saturated brine (3×30 mL), dried over anhydrous sodium sulfate, separated and purified by column chromatography (dichloromethane:methanol=12:1), and then concentrated to obtain the off-white solid of compound K-047 (60 mg), MS: 719.24 [M+H]⁺

### Example 22: Synthesis of Compound K-010

### (2-((5-Bromo-2-((5-ethyl-2-methoxy-4-(4-(4-methylpiperazin-1-yl)piperidin-1-yl)phenyl)amino)pyrimidin-4-yl)amino)-5-(5-ethynylpyrazin-2-yl)phenyl)dimethylphosphine oxide

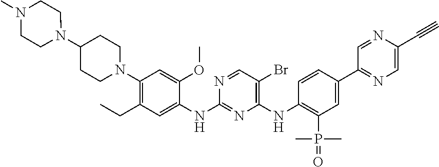 [SMILES: C#Cc1cnc(-c2ccc(Nc3nc(Nc4cc(CC)c(N5CCC(N6CCN(C)CC6)CC5)cc4OC)ncc3Br)c(P(C)(C)=O)c2)cn1]

### Step 1: Synthesis of Compound K-010-2

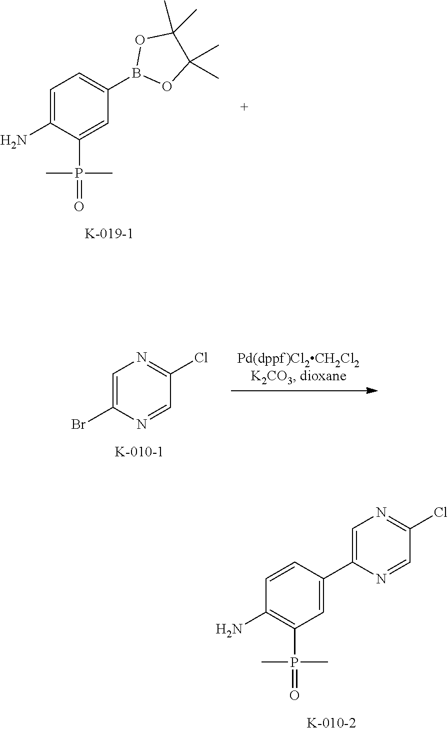 [SMILES: CC1(C)OB(c2ccc(N)c(P(C)(C)=O)c2)OC1(C)C ; Clc1cnc(Br)cn1 ; "Pd(dppf)Cl2·CH2Cl2K2CO3, dioxane" ; CP(C)(=O)c1cc(-c2cnc(Cl)cn2)ccc1N]

The compound K-010-2 was synthesized as the method described for compound K-047-2 using compound K-010-1 instead of compound K-047-1, compound K-019-1 instead of phenylboronic acid. For compound K-010-2, MS: 282.05 [M+H]⁺

### Step 2: Synthesis of Compound K-010-4

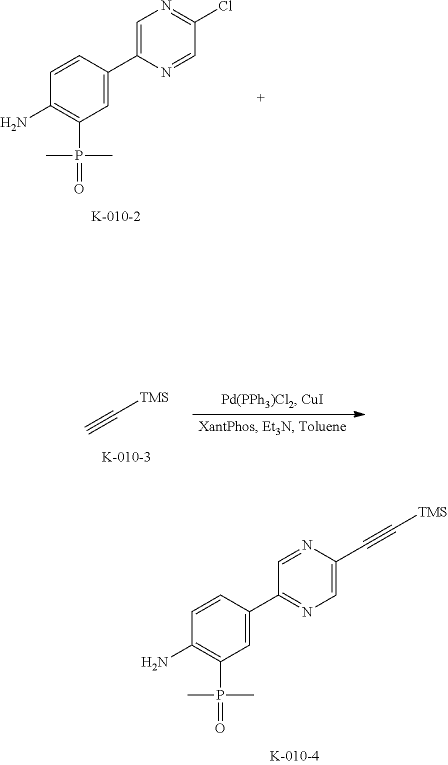

Add compounds K-010-2 (290 mg) K-0-3 (1.0 g), Pd(PPh₃)Cl₂ (72 mg), XantPhos (119 mg), CuI (59 mg), Et₃N (313 mg) and toluene (5 mL) to the reaction flask, and the gas in reactor was replaced with nitrogen for three times. Heat to 110° C. and stir for 12 hr. The reaction was complete as monitored by LCMS, and then stopped. The excess solvent was removed by rotary evaporator. And then the solution was separated and purified by column chromatography (dichloromethane:methanol=20:1) to obtain the yellowish solid of desired product compound K-00-4, MS: 344.13 [M+H]⁺

### Step 2: Synthesis of Compound K-010-4

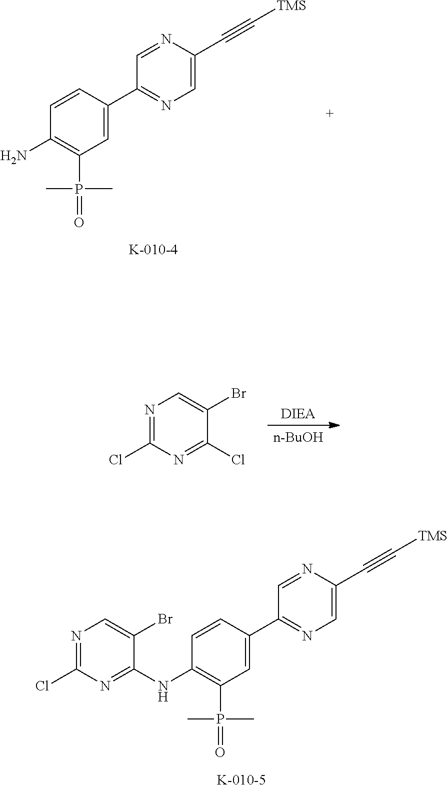

The compound K-010-5 was synthesized as the method described for compound K-004-5 using compound K-010-4 instead of raw compound K-0044. For compound K-30-4, MS: 534.02 [M+H]⁺

### Step 4: Synthesis of Compound K-010-6

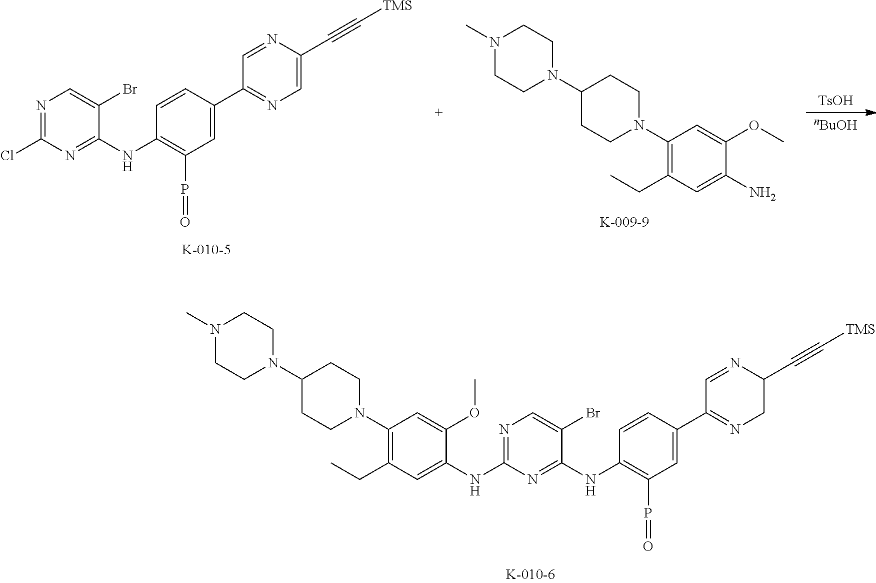

The compound K-010-6 was synthesized as the method described for compound K-018 using compound K-009-9 instead of raw compound K-018-9, compound K-010-5 instead of raw compound K-018-5. For compound K-010-6, MS: 830.30 [M+H]⁺

### Step 5: Synthesis of Compound K-010

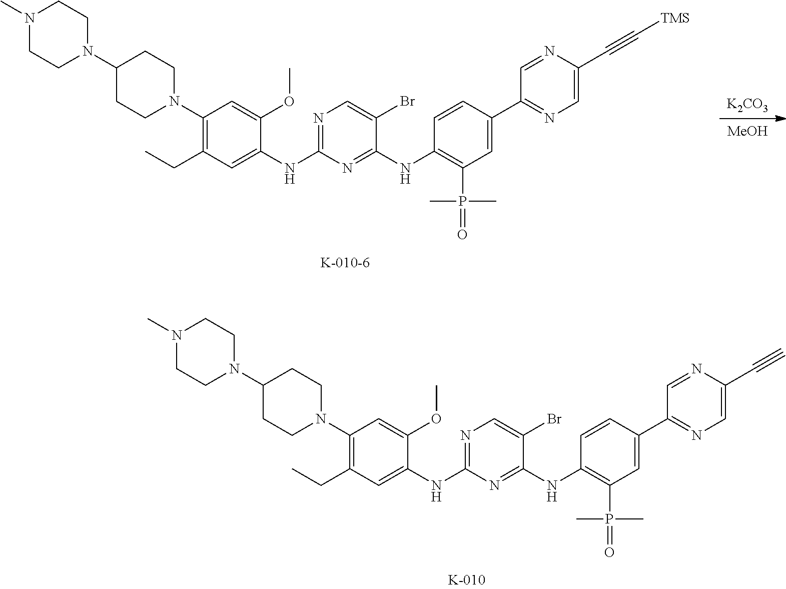 [SMILES: "MeOH" ; CCc1cc(Nc2ncc(Br)c(Nc3ccc(-c4cnc(C#C[Si](C)(C)C)cn4)cc3P(C)(C)=O)n2)c(OC)cc1N1CCC(N2CCN(C)CC2)CC1 ; "K2CO3" ; C#Cc1cnc(-c2ccc(Nc3nc(Nc4cc(CC)c(N5CCC(N6CCN(C)CC6)CC5)cc4OC)ncc3Br)c(P(C)(C)=O)c2)cn1]

Add compound K-010-6 (54 mg), K₂CO₃ (18 mg) and MeOH (3 mL) to the reaction flask, and stir at room temperature for 0.5 hr. The reaction was complete as monitored by LCMS, and then stopped. The excess solvent was removed by rotary evaporator. And then thin layer chromatography (dichloromethane:methanol=8:1) was prepared for separation and purification of the solution to obtain the yellowish solid of desired product compound K-010 (44 mg), MS: 758.26 [M+H]⁺

### Example 23: Synthesis of Compound K-011

### (2-((5-Bromo-2-((2-methoxy-5-methyl-4-(4-(4-methylpiperazin-1-yl)piperidin-1-yl)phenyl))amino)pyrimidin-4-yl)amino)-5-(5-ethynylpyrazin-2-yl)phenyl)dimethylphosphine oxide

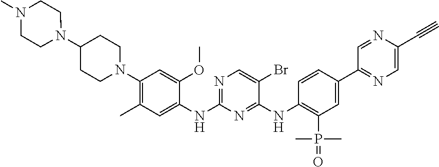 [SMILES: C#Cc1cnc(-c2ccc(Nc3nc(Nc4cc(C)c(N5CCC(N6CCN(C)CC6)CC5)cc4OC)ncc3Br)c(P(C)(C)=O)c2)cn1]

### Step 1: Synthesis of Compound K-011-3

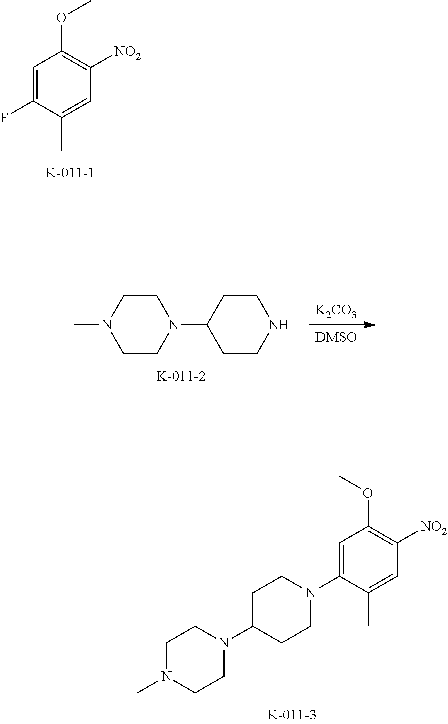

Add compounds K-011-1 (3.00 g), K-011-2 (4.13 g), K₂CO₃ (6.24 g) and DMSO (30 mL) to the reaction flask, heat to 90° C., and stir with heating for 12 hr. The reaction was complete as monitored by LCMS, and then stopped. The reaction solution was poured into water (100 mL), and filtered with suction; the filter cake was washed with water and dried to obtain the yellow solid of desired product compound K-011-3 (4.10 g), MS: 349.22 [M+H]⁺

### Step 2: Synthesis of Compound K-011-4

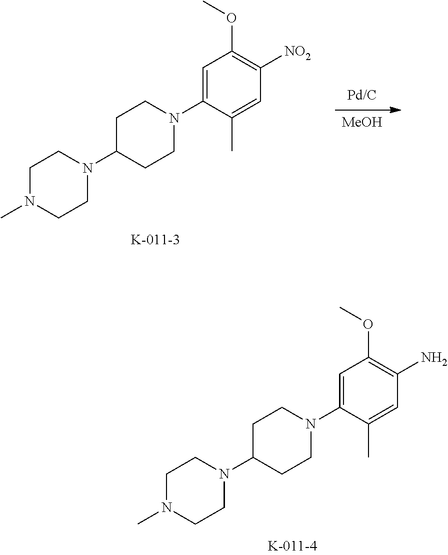 [SMILES: "MeOH" ; COc1cc(N2CCC(N3CCN(C)CC3)CC2)c(C)cc1[N+](=O)[O-] ; "Pd/C" ; COc1cc(N2CCC(N3CCN(C)CC3)CC2)c(C)cc1N]

Add compound K-011-3 (4.10 g), Pd/C (1.00 g, 10%) and MeOH (60 mL) to the reaction flask, introduce 112, and stir with heating at room temperature for 3 hr, the reaction was complete as monitored by LCMS, and then stopped. The solution was filtered with suction, and rinsed with methanol (20 mL); collect the organic phase, remove the solvent, and obtain the reddish-brown liquid of desired product compound K-011-4 (3.30 g), MS: 319.24 [M+H]⁺

### Step 3: Synthesis of Compound K-011-5

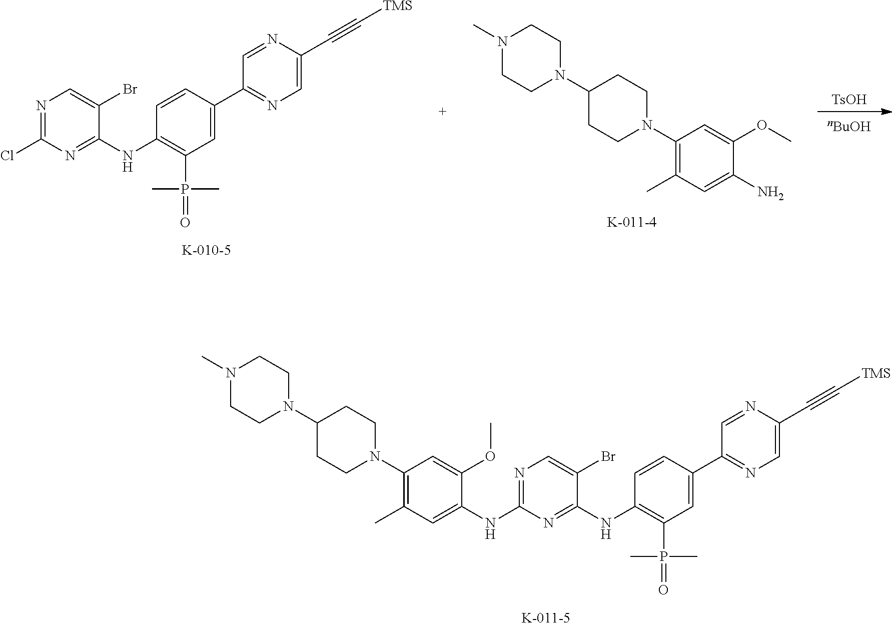

The compound K-011-5 was synthesized as the method described for compound K-018 using compound K-011-4 instead of raw compound K-018-9, compound K-010-5 instead of raw compound K-018-5. For compound K-011-5, MS: 816.29 [M+H]⁺

### Step 2: Synthesis of Compound K-011

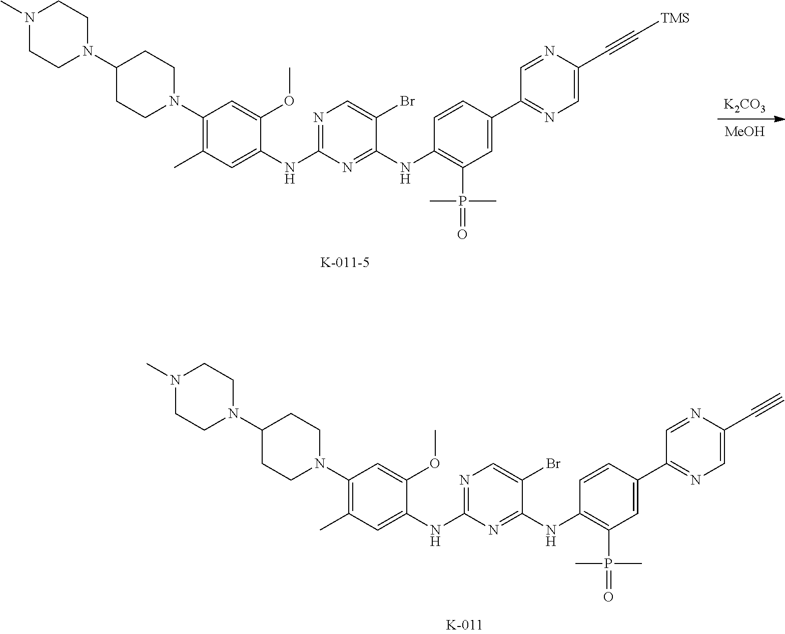 [SMILES: "MeOH" ; COc1cc(N2CCC(N3CCN(C)CC3)CC2)c(C)cc1Nc1ncc(Br)c(Nc2ccc(-c3cnc(C#C[Si](C)(C)C)cn3)cc2P(C)(C)=O)n1 ; "K2CO3" ; C#Cc1cnc(-c2ccc(Nc3nc(Nc4cc(C)c(N5CCC(N6CCN(C)CC6)CC5)cc4OC)ncc3Br)c(P(C)(C)=O)c2)cn1]

The compound K-011 was synthesized as the method described for compound K-010 using compound K-011-5 instead of raw compound K-010-6, For compound K-011, MS: 744.25 [M+H]⁺

### Example 24: Synthesis of Compound K-014

### (2-((5-Bromo-2-((5-methoxy-2-methyl-4-(4-(4-methylpiperazin-1-yl)piperidin-1-yl)phenyl)imino)pyrimidin-4-yl)amino)-5-(5-ethynylpyrazin-2-yl)phenyl)dimethylphosphine oxide

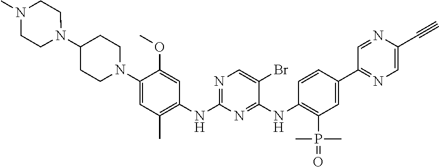 [SMILES: C#Cc1cnc(-c2ccc(Nc3nc(Nc4cc(OC)c(N5CCC(N6CCN(C)CC6)CC5)cc4C)ncc3Br)c(P(C)(C)=O)c2)cn1]

### Step 1: Synthesis of Compound K-014-1

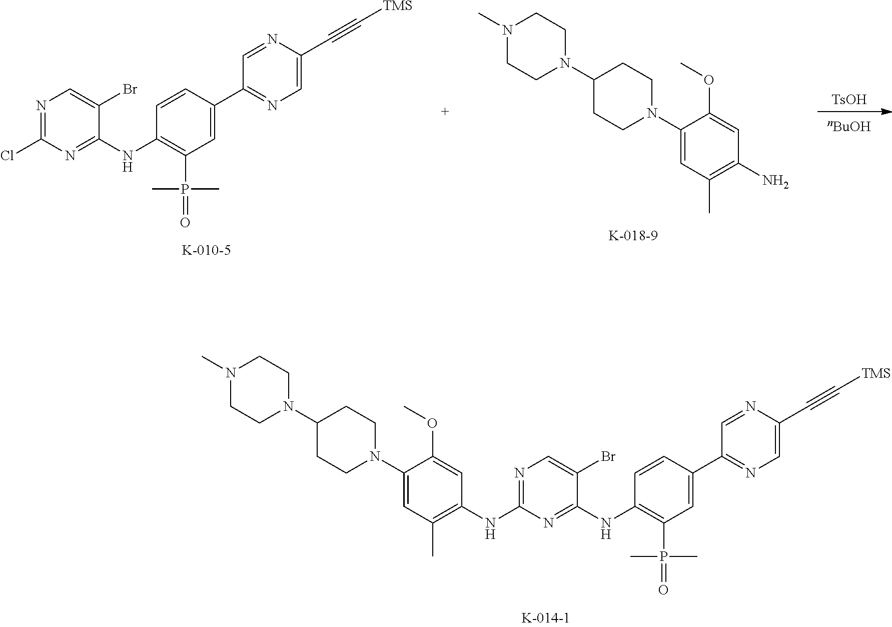

The compound K-014-1 was synthesized as the method described for compound K-018 using compound K-010-5 instead of raw compound K-018-5. For compound K-014-1, MS: 816.29 [M+H]⁺

### Step 2: Synthesis of Compound K-014

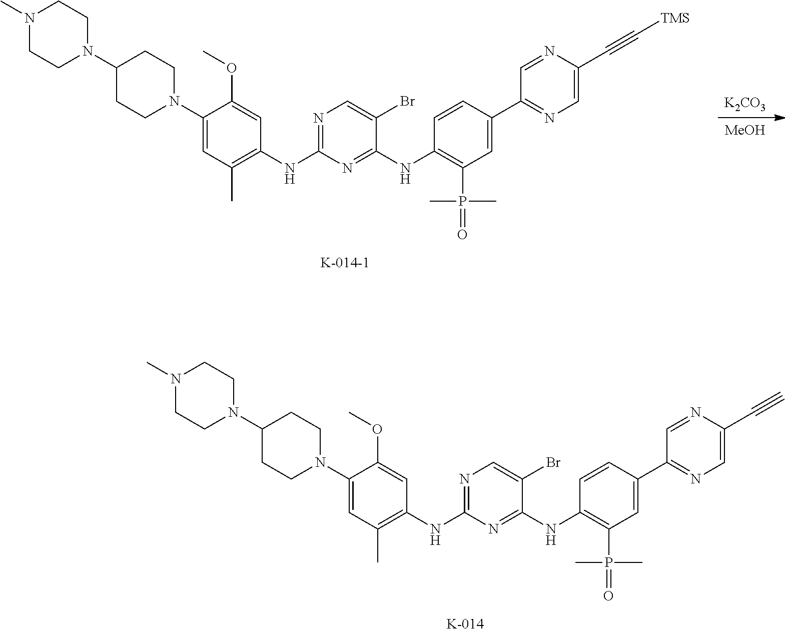 [SMILES: "MeOH" ; COc1cc(Nc2ncc(Br)c(Nc3ccc(-c4cnc(C#C[Si](C)(C)C)cn4)cc3P(C)(C)=O)n2)c(C)cc1N1CCC(N2CCN(C)CC2)CC1 ; "K2CO3" ; C#Cc1cnc(-c2ccc(Nc3nc(Nc4cc(OC)c(N5CCC(N6CCN(C)CC6)CC5)cc4C)ncc3Br)c(P(C)(C)=O)c2)cn1]

The compound K-014 was synthesized as the method described for compound K-010 using compound K-014-1 instead of raw compound K-010-6. For compound K-014, MS: 744.25 [M+H]⁺

¹H NMR (500 MHz, DMSO-d₆) δ 11.39 (s, 1H), 9.31 (d, J=1.6 Hz, 1H), 8.79 (s, 2H), 8.44 (s, 1H), 8.21-8.14 (m, 2H), 7.85 (s, 1H), 6.84 (s, 1H), 6.77 (s, 1H), 4.75 (s, 1H), 3.65 (s, 3H), 3.42 (d, J=10.9 Hz, 2H), 3.34 (s, 6H), 2.69-2.51 (m, 4H), 2.30 (s, 4H), 2.07 (s, 3H), 1.86 (d, J=13.6 Hz, 6H), 1.79 (s, 211), 1.57 (s, 211).

### Example 25: Synthesis of Compound K-013

### (2-((5-Bromo-2-((5-ethyl-2-methoxy-4-(4-(4-methylpiperazin-1-yl)piperidin-1-yl)phenyl)amino)pyrimidin-4-yl)amino)-5-(6-ethynylpyrazin-2-yl)phenyl)dimethylphosphine oxide

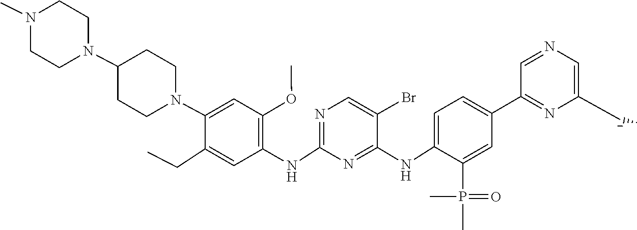

### Step 1: Synthesis of Compound K-013-2

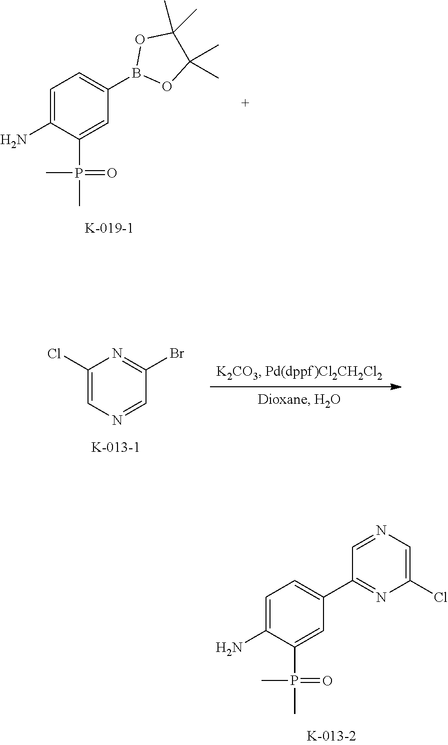 [SMILES: CC1(C)OB(c2ccc(N)c(P(C)(C)=O)c2)OC1(C)C ; Clc1cncc(Br)n1 ; "Dioxane, H2O" ; "K2CO3, Pd(dppf)Cl2CH2Cl2" ; CP(C)(=O)c1cc(-c2cncc(Cl)n2)ccc1N]

The compound K-013-2 was synthesized as the method described for compound K-047-2 using compound K-013-1 instead of compound K-047-1, compound K-019-1 instead of phenylboronic acid. For compound K-013-2. MS: 282.05 [M+H]⁺

### Step 2: Synthesis of Compound K-013-3

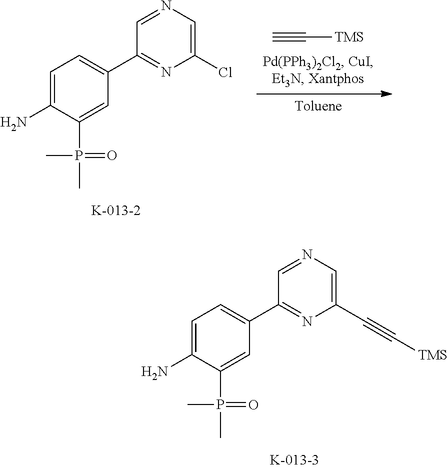

Add K-013-2 (380 mg), trimethylsilylacetylene (1.9 mL), Xantphos (156 mg), Pd(PPh₃)₂Cl₂ (95 mg), CuI (77 mg). Et₃N (410 mg) and methylbenzene (10 mL) to the reaction flask, heat to 100° C., and react for 12 hr under nitrogen protection. After complete reaction, the solution was treated with column chromatography (dichloromethane:methanol=10:1) to obtain the red powder compound K-013-3 (409 mg), MS: 344.13 [M+H]⁺

### Step 3: Synthesis of Compound K-013-4

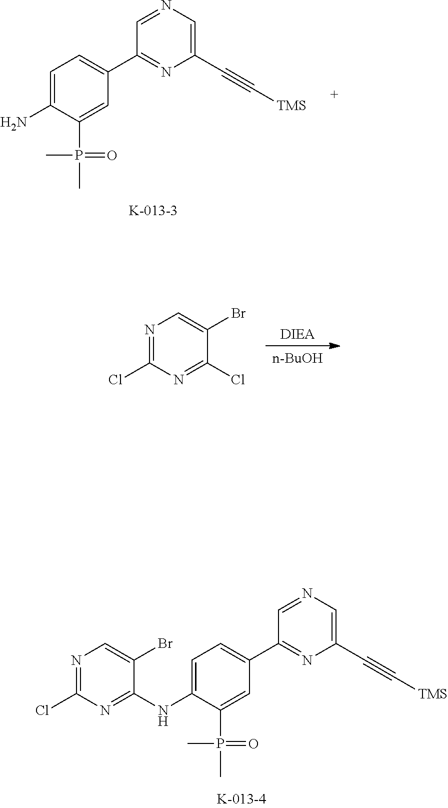

The compound K-013-4 was synthesized as the method described for compound K-004-5 using compound K-013-3 instead of raw compound K-004-4. For compound K-013-4, MS: 534.02 [M+H]⁺

### Step 4: Synthesis of Compound K-013-5

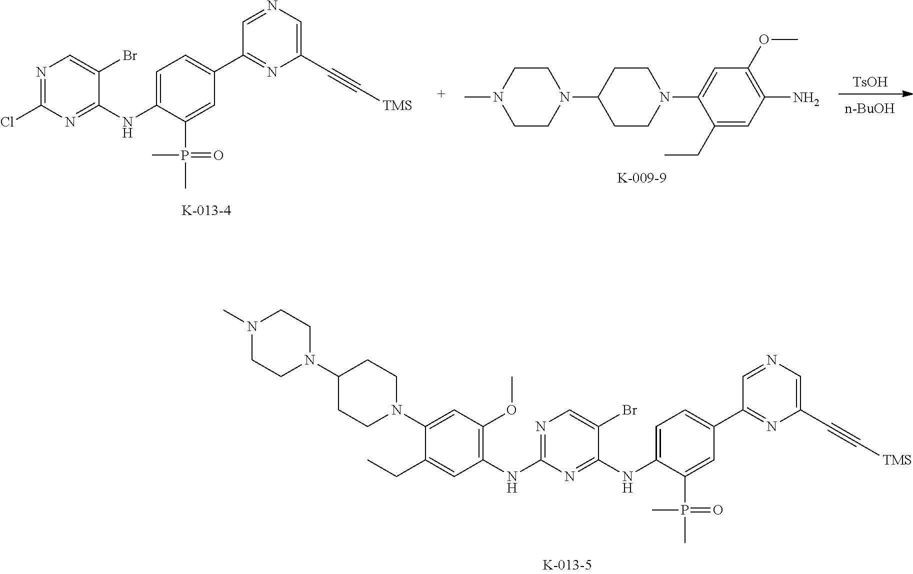

The compound K-013-5 was synthesized as the method described for compound K-018 using compound K-009-9 instead of raw compound K-018-9, compound K-013-4 instead of raw compound K-018-5. For compound K-013-5, MS: 830.30 [M+H]⁺

### Step 5: Synthesis of Compound K-013

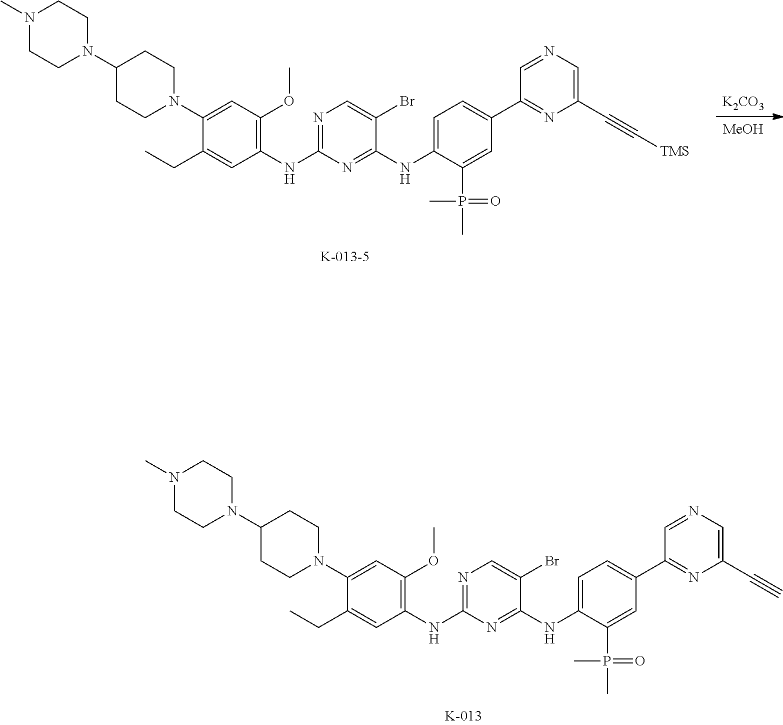 [SMILES: "MeOH" ; CCc1cc(Nc2ncc(Br)c(Nc3ccc(-c4cncc(C#C[Si](C)(C)C)n4)cc3P(C)(C)=O)n2)c(OC)cc1N1CCC(N2CCN(C)CC2)CC1 ; "K2CO3" ; C#Cc1cncc(-c2ccc(Nc3nc(Nc4cc(CC)c(N5CCC(N6CCN(C)CC6)CC5)cc4OC)ncc3Br)c(P(C)(C)=O)c2)n1]

The compound K-013 was synthesized as the method described for compound K-010 using compound K-013-5 instead of raw compound K-010-6. For compound K-013, MS: 758.26 [M+H]⁺

### Example 26: Synthesis of Compound K-015

### (2-((5-Bromo-2-((5-methoxy-2-methyl-4-morpholinophenyl)amino)pyrimidin-4-yl)amino)-5-(2-methyl-2H-1,2,3-triazol-4-yl)phenyl)dimethylphosphine oxide

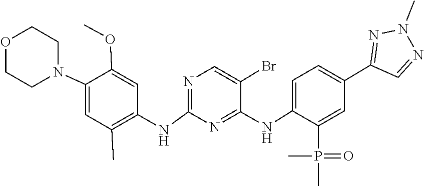 [SMILES: COc1cc(Nc2ncc(Br)c(Nc3ccc(-c4cnn(C)n4)cc3P(C)(C)=O)n2)c(C)cc1N1CCOCC1]

### Step 1: Synthesis of Compound K-015-2

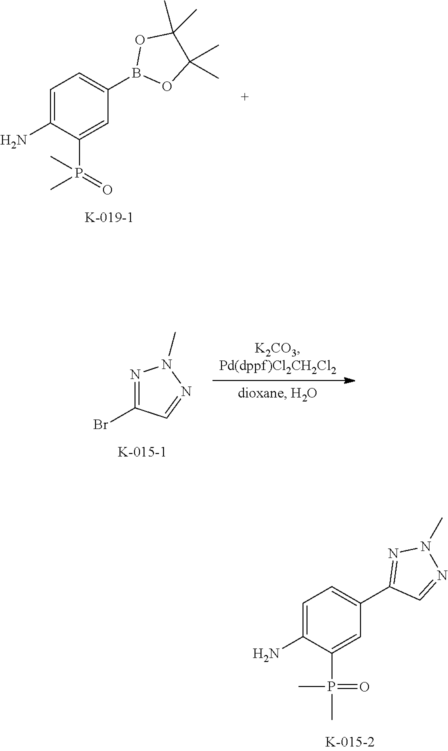

The compound K-015-2 was synthesized as the method described for compound K-047-2 using compound K-015-1 instead of compound K-047-1, compound K-019-1 instead of phenylboronic acid, to obtain the brown powder compound K-015-2 (330 mg). For compound K-015-2, MS: 251.10 [M+H]⁺

### Step 2; Synthesis of Compound K-015-3

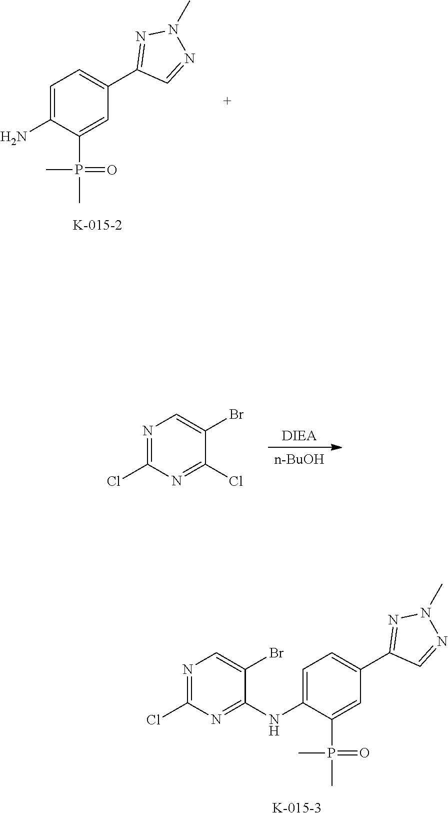

The compound K-015-3 was synthesized as the method described for compound K-004-5 using compound K-015-3 instead of raw compound K-004-4 to obtain the off-white powder of compound K-015-3 (343 mg). For compound K-015-3, MS: 440.99 [M+H]⁺

### Step 3: Synthesis of Compound K-015

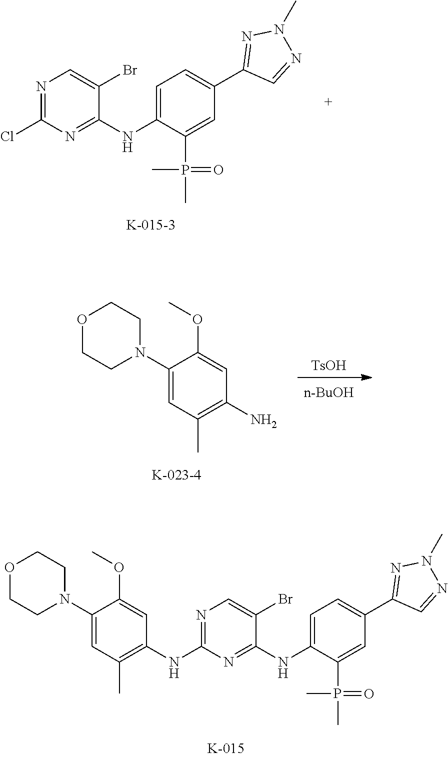

The compound K-015 was synthesized as the method described for compound K-018 using compound K-023-4 instead of raw compound K-018-9, compound K-015-3 instead of raw compound K-018-5. For compound K-015, MS: 627.15 [M+H]⁺

### Example 27: Synthesis of Compound K-040

### (2-((5-Bromo-2-((5-ethyl-2-methoxy-4-(4-morpholinopiperidin-1-yl)phenyl)amino)pyrimidin-4-yl)amino)-5-(2-methyl-2H-1,2,3-triazol-4-yl)phenyl)dimethylphosphine oxide

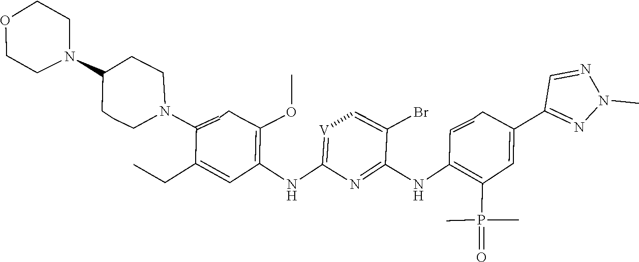

### Step 1: Synthesis of Compound K-040

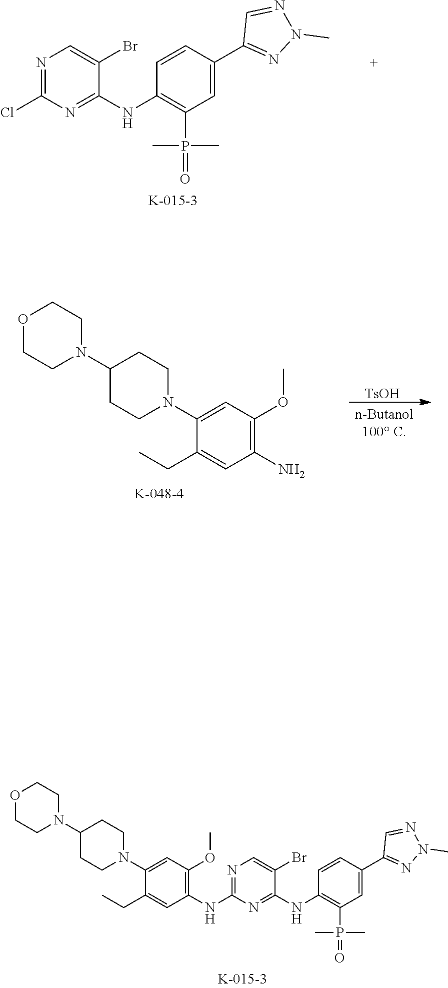 [SMILES: Cn1ncc(-c2ccc(Nc3nc(Cl)ncc3Br)c(P(C)(C)=O)c2)n1 ; CCc1cc(N)c(OC)cc1N1CCC(N2CCOCC2)CC1 ; "TsOH" ; CCc1cc(Nc2ncc(Br)c(Nc3ccc(-c4cnn(C)n4)cc3P(C)(C)=O)n2)c(OC)cc1N1CCC(N2CCOCC2)CC1]

The compound K-040 was synthesized as the method described for compound K-018 using compound K-048-4 instead of raw compound K-018-9, compound K-015-3 instead of raw compound K-018-5. For compound K-040, MS: 724.24 [M+H]⁺

### Example 28: Synthesis of Compound K-021

### (2-((5-Bromo-2-((5-methoxy-2-methyl-4-(4-morpholinopiperidin-1-yl)phenyl)amino)pyrimidin-4-yl)amino)-5-(2-methyl-2H-1,2,3-triazol-4-yl)phenyl)dimethylphosphine oxide

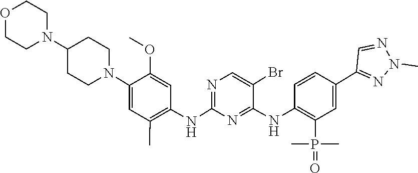 [SMILES: COc1cc(Nc2ncc(Br)c(Nc3ccc(-c4cnn(C)n4)cc3P(C)(C)=O)n2)c(C)cc1N1CCC(N2CCOCC2)CC1]

### Step 1: Synthesis of Compound K-021-3

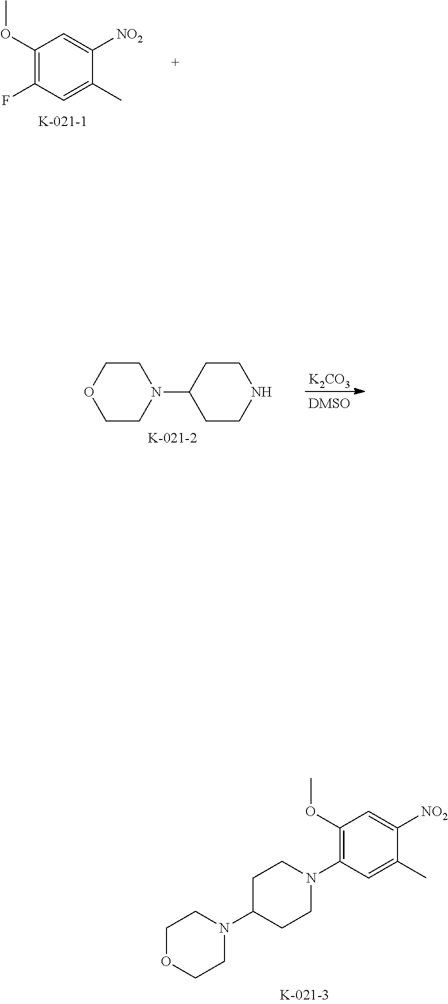

Add K-021-1 (3.00 g). K-021-2 (4.11 g), K₂CO₃ (6.24 g) and DMSO (30 mL) to the reaction flask, raise temperature to 90° C., and stir with heating for 12 hr. The reaction was complete as monitored by LCMS, and then stopped. The reaction solution was poured into water (100 mL), and filtered with suction; the filter cake was washed with water and dried to obtain the yellow solid of desired product K-021-3 (4.00 g), MS: 336.18 [M+H]⁺

### Step 2: Synthesis of Compound K-021-4

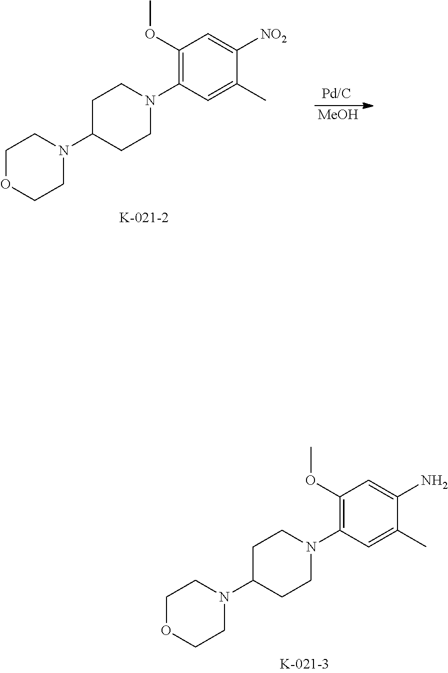 [SMILES: "MeOH" ; COc1cc([N+](=O)[O-])c(C)cc1N1CCC(N2CCOCC2)CC1 ; COc1cc(N)c(C)cc1N1CCC(N2CCOCC2)CC1]

Add compound K-021-2 (4.20 g), Pd/C (1.00 g, 10%) and MeOH (60 mL) to the reaction flask, introduce 112, and stir at room temperature for 3 hr, the reaction was complete as monitored by LCMS, and then stopped. The solution was filtered with suction, and rinsed with methanol (20 mL); collect the organic phase, remove the solvent, and obtain the reddish-brown liquid of desired product compound K-021-3 (3.2 g), MS: 306.21 [M+H]⁺

### Step 3: Synthesis of Compound K-021

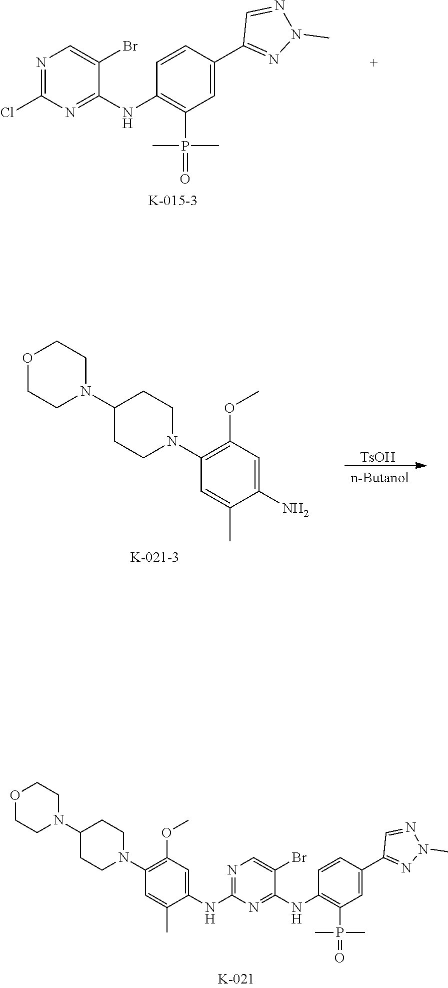 [SMILES: Cn1ncc(-c2ccc(Nc3nc(Cl)ncc3Br)c(P(C)(C)=O)c2)n1 ; COc1cc(N)c(C)cc1N1CCC(N2CCOCC2)CC1 ; "TsOH" ; COc1cc(Nc2ncc(Br)c(Nc3ccc(-c4cnn(C)n4)cc3P(C)(C)=O)n2)c(C)cc1N1CCC(N2CCOCC2)CC1]

The compound K-021 was synthesized as the method described for compound K-018 using compound K-021-3 instead of raw compound K-018-9, compound K-015-3 instead of raw compound K-018-5. For compound K-021, MS: 710.23 [M+H]⁺

¹H NMR (500 MHz, DMSO-d6) δ 11.22 (s, 1H), 8.74 (s, 1H), 8.39 (s, 1H), 8.22 (s, 1H), 8.16 (s, 1H), 7.83 (d, J=14.3 Hz, 1H), 7.54 (s, 1H), 6.85 (s, 1H), 6.78 (s, 1H), 4.19 (s, 3H), 3.66 (s, 3H), 3.59 (s, 6H), 3.46 (s, 2H), 2.58 (t, J=11.6 Hz, 4H), 2.26 (s, 1H), 2.07 (s, 3H), 1.83 (d, J=13.6 Hz, 8H), 1.59 (s, 2H).

### Example 29: Synthesis of Compound K-008

### (2-((5-Bromo-2-((2-methoxy-5-methyl-4-(4-(4-methylpiperazin-1-yl)piperidin-1-yl)phenyl)amino)pyrimidin-4-yl)amino)-5-(2-methyl-2H-1,2,3-triazol-4-yl)phenyl)dimethylphosphine oxide

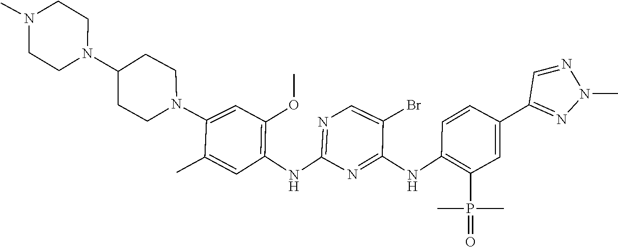

### Step 1: Synthesis of Compound K-008

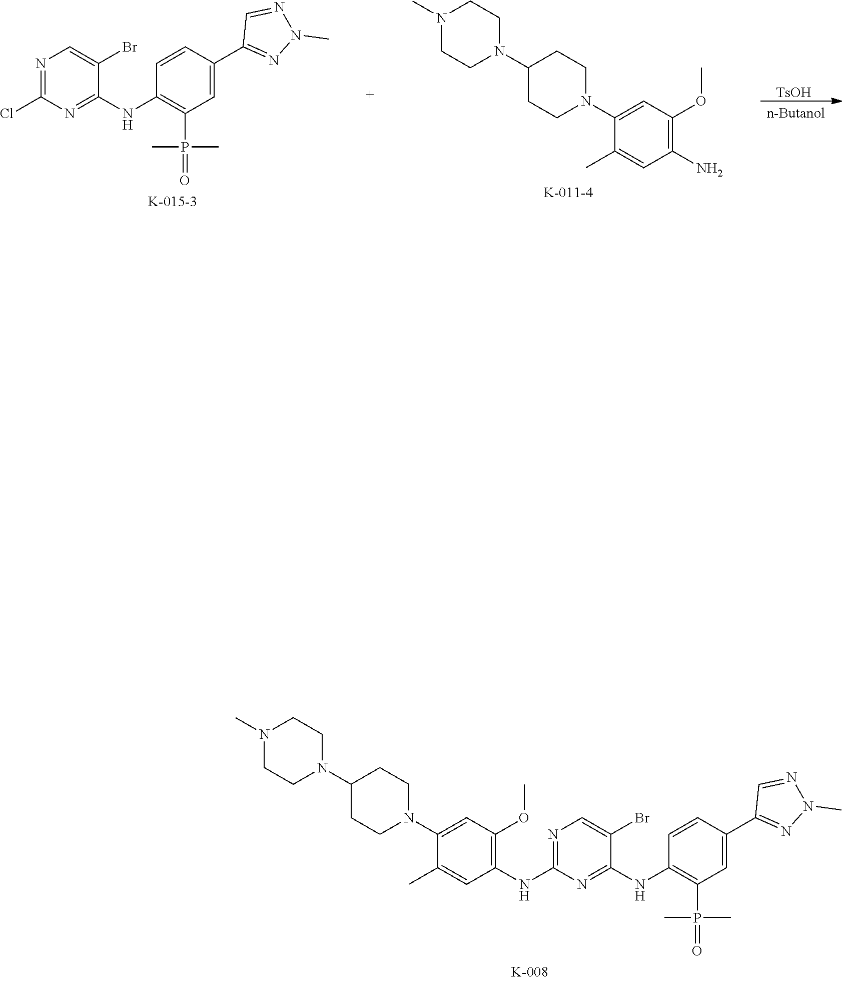

The compound K-008 was synthesized as the method described for compound K-018 using compound K-011-4 instead of raw compound K-018-9, compound K-015-3 instead of raw compound K-018-5. For compound K-008, MS: 723.26 [M+H]⁺

### Example 30: Synthesis of Compound K-049

### (4-((5-Bromo-2-((5-ethyl-2-methoxy-4-(4-morpholinopiperidin-1-yl)phenyl)amino)pyrimidin-4-yl)amino)-2′-methyl-[1,1′-biphenyl]-3-yl)dimethylphosphine oxide

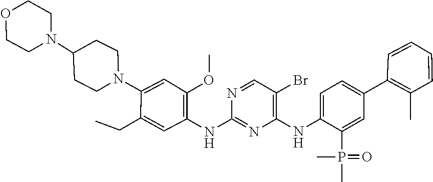 [SMILES: CCc1cc(Nc2ncc(Br)c(Nc3ccc(-c4ccccc4C)cc3P(C)(C)=O)n2)c(OC)cc1N1CCC(N2CCOCC2)CC1]

### Step 1: Synthesis of Compound K-049-1

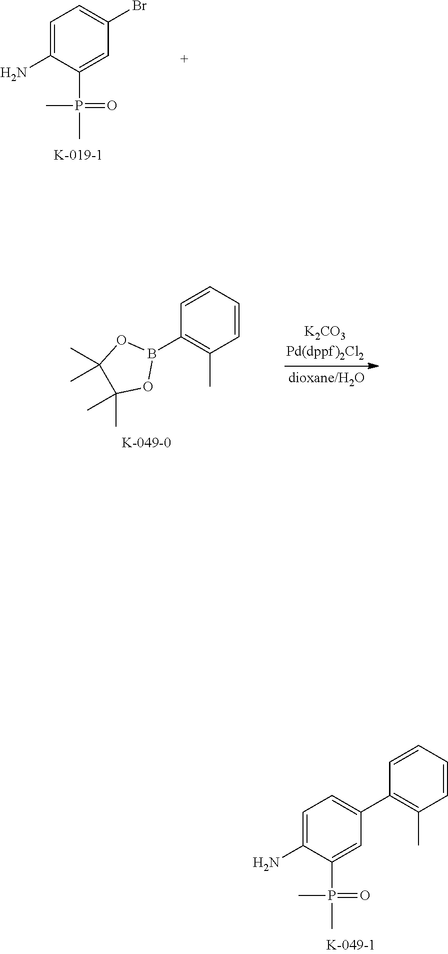 [SMILES: CP(C)(=O)c1cc(Br)ccc1N ; Cc1ccccc1B1OC(C)(C)C(C)(C)O1 ; Cc1ccccc1-c1ccc(N)c(P(C)(C)=O)c1]

The compound K-049-1 was synthesized as the method described for compound K-047-2 using compound K-019-1 instead of compound K-047-1, compound K-049-0 instead of phenylboronic acid. For compound K-049-1, MS: 260.11 [M+H]⁺

### Step 2: Synthesis of Compound K-049-2

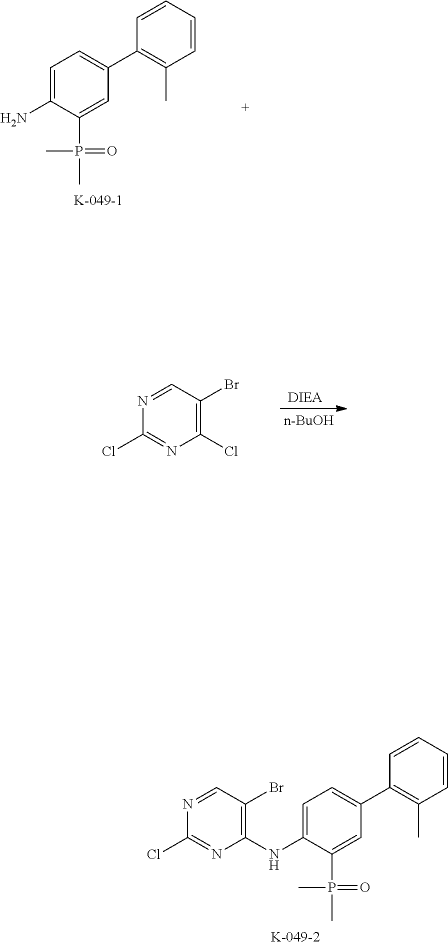

The compound K-049-2 was synthesized as the method described for compound K-004-5 using compound K-049-1 instead of raw compound K-004-4. For compound K-049-2, MS: 450.01 [M+H]⁺

### Step 3: Synthesis of Compound K-049

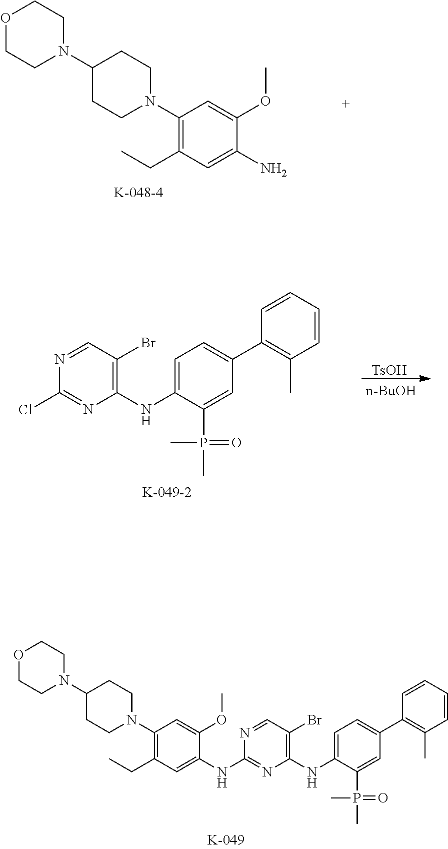

The compound K-049-5 was synthesized as the method described for compound K-018 using compound K-048-4 instead of raw compound K-018-9, compound K-049-2 instead of raw compound K-018-5. For compound K-049, MS: 733.26 [M+H]⁺

### Example 31: Synthesis of Compound K-007

### (6-((5-Bromo-2-((5-ethyl-2-methoxy-4-(4-(4-methylpiperazin-1-yl)piperidin-1-yl)phenyl)amino)pyrimidin-4-yl)amino)-2-fluoro-3-(2-methyl-2H-1,2,3-triazol-4-yl)phenyl)dimethylphosphine oxide

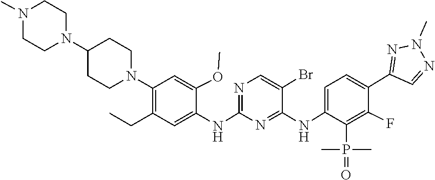 [SMILES: CCc1cc(Nc2ncc(Br)c(Nc3ccc(-c4cnn(C)n4)c(F)c3P(C)(C)=O)n2)c(OC)cc1N1CCC(N2CCN(C)CC2)CC1]

### Step 1: Synthesis of Compound K-007-3

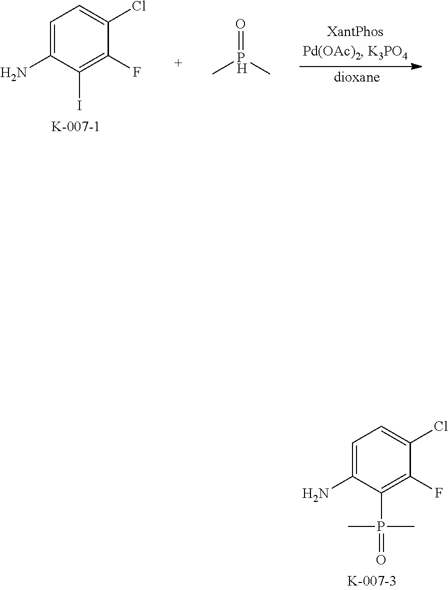

Add compound K-007-1 (500 mg), dimethylphosphine oxide (216 mg), Pd(OAc)₂ (41 mg), Xantphos (107 mg), K₃PO₄ (586 mg) and 1,4-Dioxane (15 mL) into the reaction flask; the gas in the reactor was replaced with nitrogen for three times; raise temperature to 100° C. and stir for 12 hr. The reaction was complete as monitored by LCMS, and then stopped. Filter the solution, remove the solvent and carry out column chromatography (dichloromethane:methanol=20:1) for separation and purification of the solution to obtain the brown solid of desired product compound K-007-3 (390 mg), MS: 222.02 [M+H]⁺

### Step 2: Synthesis of Compound K-007-6

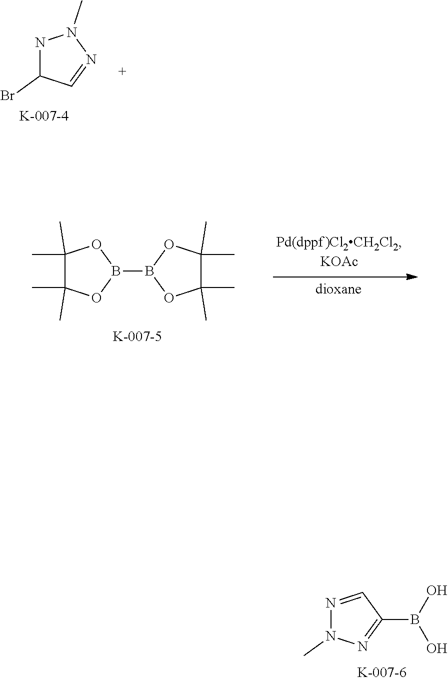

Add compounds K-007-4 (500 mg), K-007-5 (784 mg), Pd(dppf)Cl₂.CH₂Cl₂ (252 mg), KOAc (606 mg) and 1,4-Dioxane (15 mL) to the reaction flask; the gas in the reactor was replaced with nitrogen for three times; raise temperature to 100° C., and stir for 12 hr. The reaction was complete as monitored by LCMS, and then stopped. Filter the solution, remove the solvent and carry out column chromatography (petroleum ether:ethyl acetate=6:1) for separation and purification of the solution to obtain the brown solid of desired product compound K-007-6 (640 mg, crude product), MS: 128.06 [M+H]⁺

### Step 3: Synthesis of Compound K-007-7

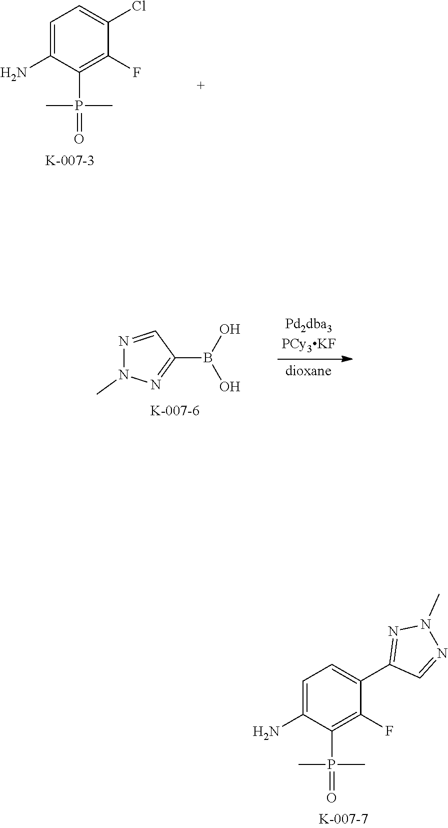

Add K-007-3 (159 mg), K-007-6 (300 mg), PCy₃ (40 mg), Pd₂(dba)₃ (66 mg), KF (125 mg) and 1,4-Dioxane (10 mL) to the reaction flask; the gas in the reactor was replaced with nitrogen for three times; raise temperature to 160° C. and keep microwave reaction for 2.5 hr. The reaction was complete as monitored by LCMS, and then stopped. Filter the solution, remove the solvent and carry out column chromatography (dichloromethane:methanol=30:1) for separation and purification of the solution to obtain the yellow solid of desired product K-007-7 (120 mg), MS: 269.09 [M+H]⁺

### Step 4: Synthesis of Compound K-007-8

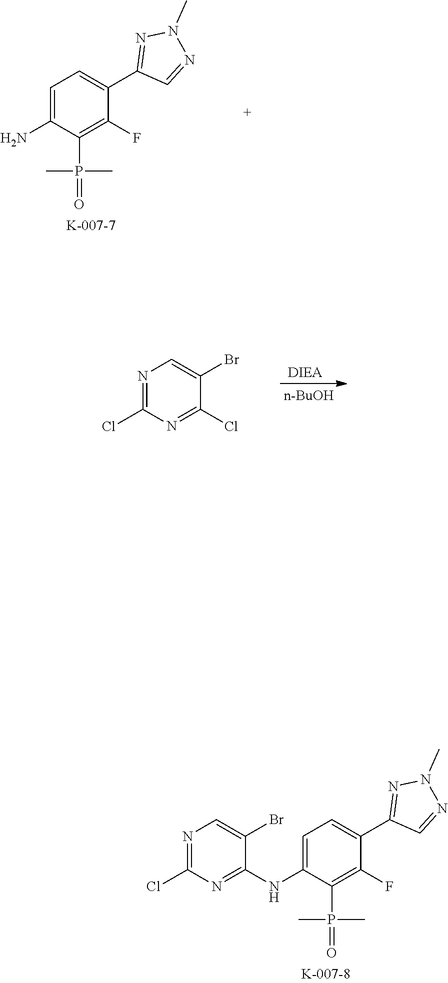

The compound K-007-8 was synthesized as the method described for compound K-004-5 using compound K-007-7 instead of raw compound K-004-4. For compound K-007-8, MS: 458.98 [M+H]⁺

### Step 5: Synthesis of Compound K-007

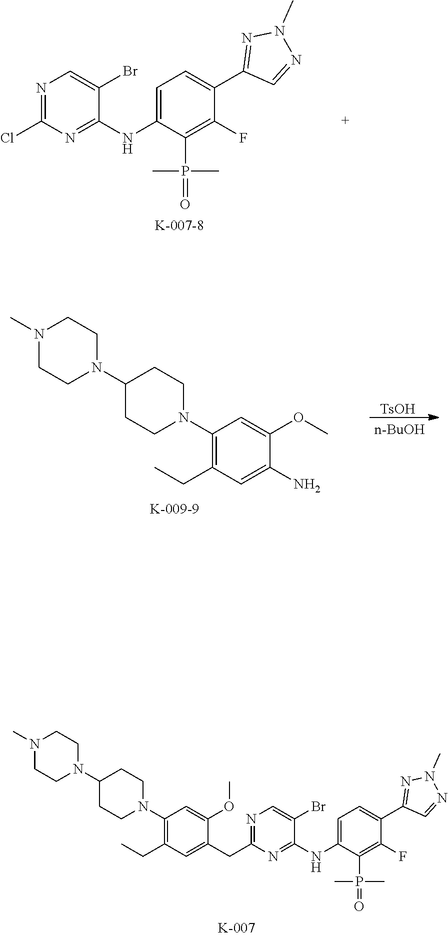

The compound K-007 was synthesized as the method described for compound K-018 using compound K-009-9 instead of raw compound K-018-9, compound K-007-8 instead of raw compound K-018-5. For compound K-007, MS: 755.26 [M+H]⁺

### Example 32: Synthesis of Compound K-037

### (6-((5-Bromo-2-((4-(2,6-dimethylmorpholine)-5-methoxy-2-methylphenyl)amino)pyrimidin-4-yl)amino)-2-fluoro-3-(2-methyl-2H-1,2,3-triazol-4-yl)phenyl)dimethylphosphine oxide

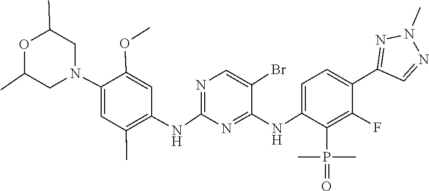 [SMILES: COc1cc(Nc2ncc(Br)c(Nc3ccc(-c4cnn(C)n4)c(F)c3P(C)(C)=O)n2)c(C)cc1N1CC(C)OC(C)C1]

### Step 1: Synthesis of Compound K-037-2

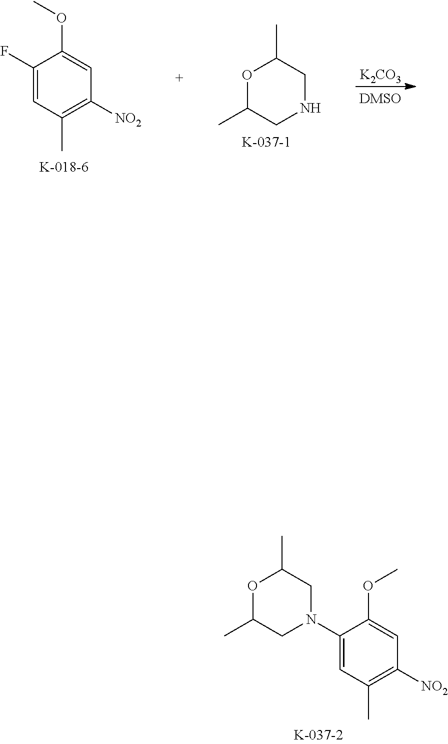

The compound K-037-2 was synthesized as the method described for compound K-018-8 using compound K-037-1 instead of raw compound K-018-7. For compound K-037-2, MS: 281.14 [M+H]⁺

### Step 2: Synthesis of Compound K-037-3

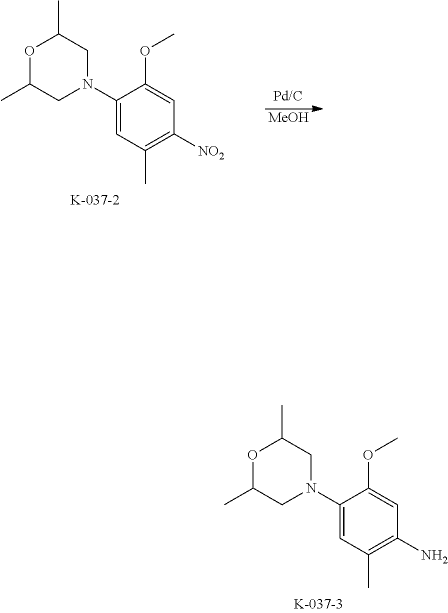 [SMILES: "MeOH" ; COc1cc([N+](=O)[O-])c(C)cc1N1CC(C)OC(C)C1 ; COc1cc(N)c(C)cc1N1CC(C)OC(C)C1]

The compound K-037-3 was synthesized as the method described for compound K-018-9 using compound K-037-2 instead of raw compound K-018-8. For compound K-037-3, MS: 251.17 [M+H]⁺

### Step 3: Synthesis of Compound K-037

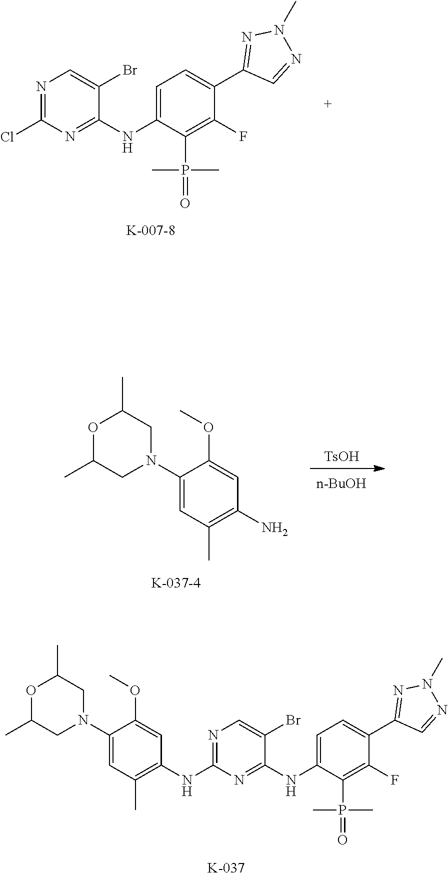

The compound K-037 was synthesized as the method described for compound K-018 using compound K-037-4 instead of raw compound K-018-9, compound K-007-8 instead of raw compound K-018-5. For compound K-037, MS: 673.17 [M+H]⁺

### Example 33: Synthesis of Compound K-025

### (6-((5-Bromo-2-((5-methoxy-2-methyl-4-morpholinophenyl)amino)pyrimidin-4-yl)amino)-2-fluoro-3-(2-methyl-2H-1,2,3-triazol-4-yl)phenyl)dimethylphosphine oxide

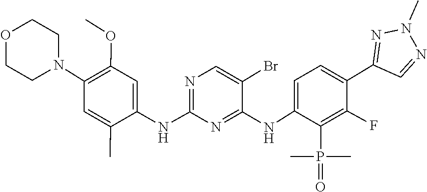 [SMILES: COc1cc(Nc2ncc(Br)c(Nc3ccc(-c4cnn(C)n4)c(F)c3P(C)(C)=O)n2)c(C)cc1N1CCOCC1]

### Step 1: Synthesis of compound K-025

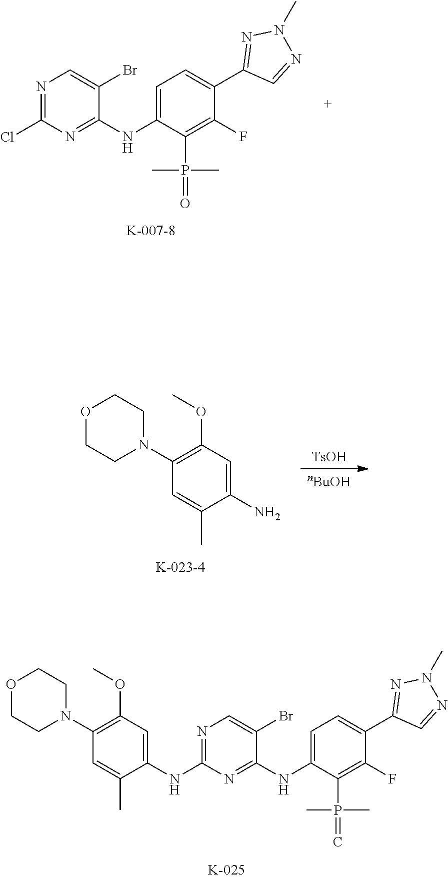

The compound K-025 was synthesized as the method described for compound K-018 using compound K-023-4 instead of raw compound K-018-9. compound K-007-8 instead of raw compound K-OI8-5. For compound K-025, MS: 645.14 [M+H]⁺

### Example 34: Synthesis of compound K-012

### (6-((5-Bromo-2-((5-methoxy-2-methyl-4-(4-(4-methylpiperazin-1-yl)piperidin-1-yl) phenyl)amino)pyrimidin-4-yl)amino)-2-fluoro-3-(2-methyl-2H-1,2,3-triazol-4-yl)ph enyl)diniethylphosphine oxide

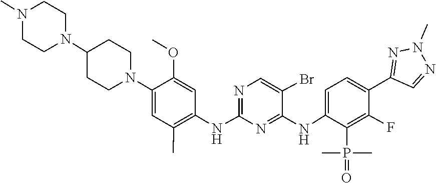 [SMILES: COc1cc(Nc2ncc(Br)c(Nc3ccc(-c4cnn(C)n4)c(F)c3P(C)(C)=O)n2)c(C)cc1N1CCC(N2CCN(C)CC2)CC1]

### Step 1: Synthesis of Compound K-012

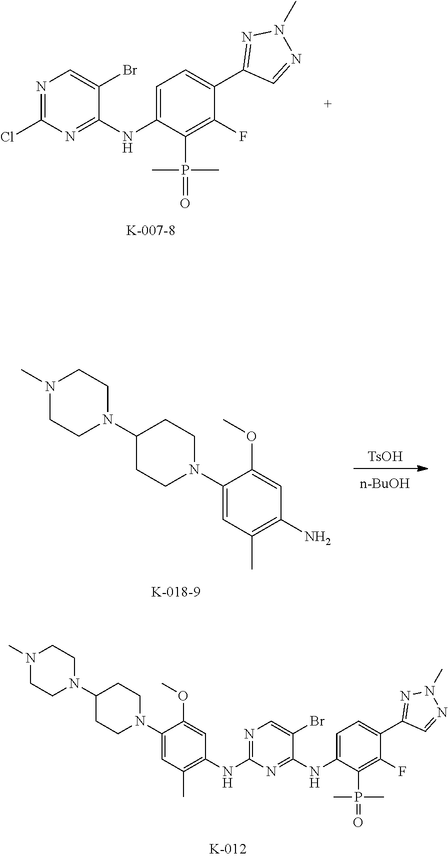

Add compounds K-007-8 (95 mg), K-018-9 (66 mg), TsOH (71 mg) and n-BuOH (3 mL) to the reaction flask, raise temperature to 115° C. and keep microwave reaction for 1.5 hr. The reaction was complete as monitored by LCMS, and then stopped. Filter the solution, remove the solvent and prepare plate (dichloromethane:methanol=8:1) for separation and purification of the solution to get the yellow solid compound K-012 (36 mg), MS: 741.25 [M+H]⁺

¹H NMR (500 MHz, DMSO-d₆) δ 11.93 (s, 1), 8.82 (s, 1H), 8.37 (s, 1H), 8.19 (s, 1H), 7.97 (d, J=3.9 Hz, 1H), 7.56 (s, 1H), 6.84 (s, 1H), 6.80 (s, 1H), 4.23 (s, 3H), 3.68 (s, 3H), 3.49 (d, J=11.0 Hz, 2H), 2.70 (m, 8H), 2.60 (t, J=11.6 Hz, 2H), 2.50 (d, J=5.8 Hz, 4H), 2.07 (s, 3H), 1.89 (dd, J=13.9, 2.3 Hz, 8H), 1.65 (s, 2H).

### Example 35: Synthesis of Compound K-022

### ((2-((5-Bromo-2-((5-methoxy-2-methyl-4-morpholinophenyl)amino)pyrimidin-4-yl)amino)-5-(pyrazin-2-yl)phenyl)dimethylphosphine oxide

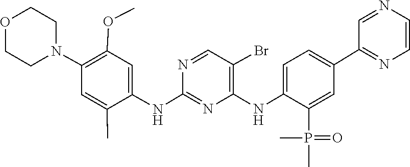 [SMILES: COc1cc(Nc2ncc(Br)c(Nc3ccc(-c4cnccn4)cc3P(C)(C)=O)n2)c(C)cc1N1CCOCC1]

### Step 1: Synthesis of Compound K-022-2

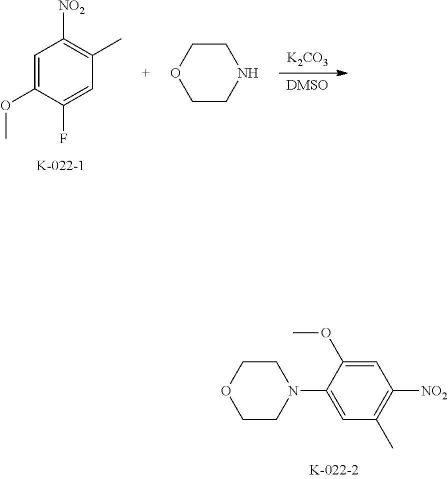

The compound K-022-2 was synthesized as the method described for compound K-018-8 using compound K-022-1 instead of raw compound K-018-6, compound morpholine instead of raw compound K-018-7. For compound K-022-2, MS: 253.11 [M+H]⁺

### Step 2: Synthesis of Compound K-022-3

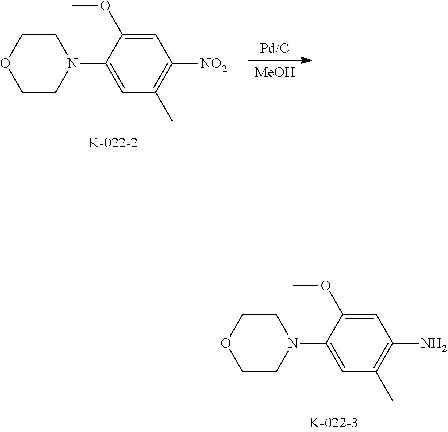 [SMILES: "MeOH" ; COc1cc([N+](=O)[O-])c(C)cc1N1CCOCC1 ; "Pd/C" ; COc1cc(N)c(C)cc1N1CCOCC1]

The compound K-022-3 was synthesized as the method described for compound K-018-9 using compound K-022-2 instead of raw compound K-018-8. For compound K-022-3, MS: 223.14 [M+H]⁺

### Step 3: Synthesis of Compound K-022-6

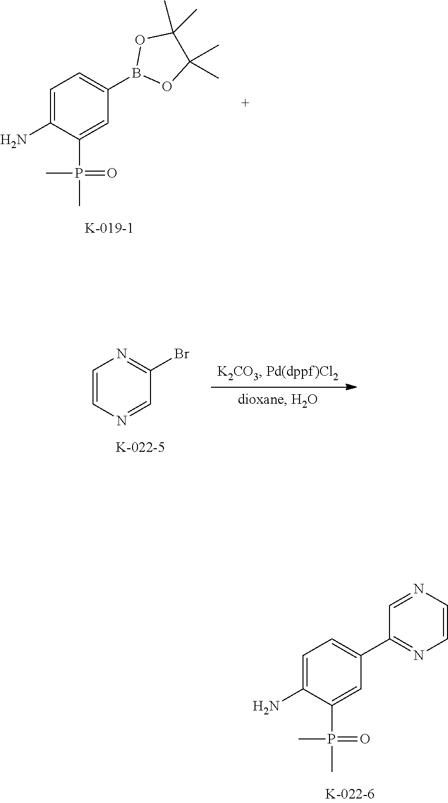 [SMILES: CC1(C)OB(c2ccc(N)c(P(C)(C)=O)c2)OC1(C)C ; Brc1cnccn1 ; "K2CO3, Pd(dppf)Cl2" ; "dioxane, H2O" ; CP(C)(=O)c1cc(-c2cnccn2)ccc1N]

Dissolve compounds K-019-1 (2.13 g, 10.00 mmol), K-022-5 (3.14 g, 20 mmol), Pd(dppf)₂Cl₂ (0.58 g, 1.0 mmol), K₂CO₃ (2.76 g, 20 mmol) in 1,4-Dioxane (20 mL) and H₂O (4 mL); heat to 100° C. under nitrogen protection, and stir for 16 hr. The reaction was complete as monitored by TLC and the solution was cooled down to room temperature. Water (30 mL) was added to the solution, which was extracted with ethyl acetate (3×30 mL); the organic phase was washed with saturated brine (3×30 ml), dried over anhydrous sodium sulfate, and purified by column chromatography (n-hexane:ethyl acetate-15:1) to get the pale yellow solid compound K-022-6 (1.5 g).

### Step 4: Synthesis of Compound K-022-7

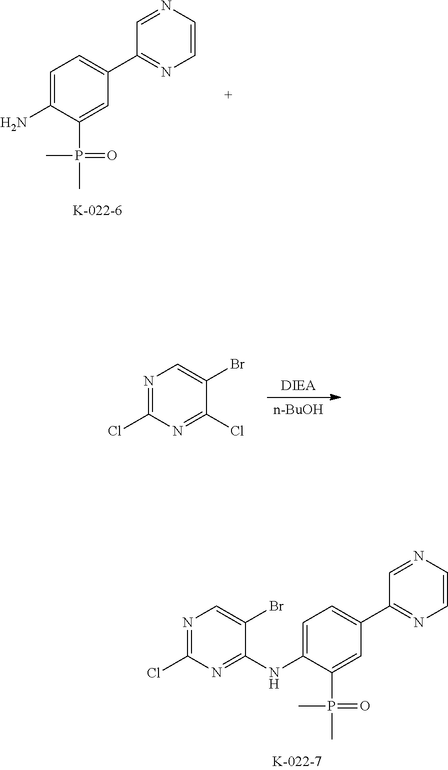

The compound K-022-7 was synthesized as the method described for compound K-004-5 using compound K-022-6 instead of raw compound K-004-4. For compound K-022-7, MS: 437.98 [M+H]⁺

### Step 5: Synthesis of Compound K-022

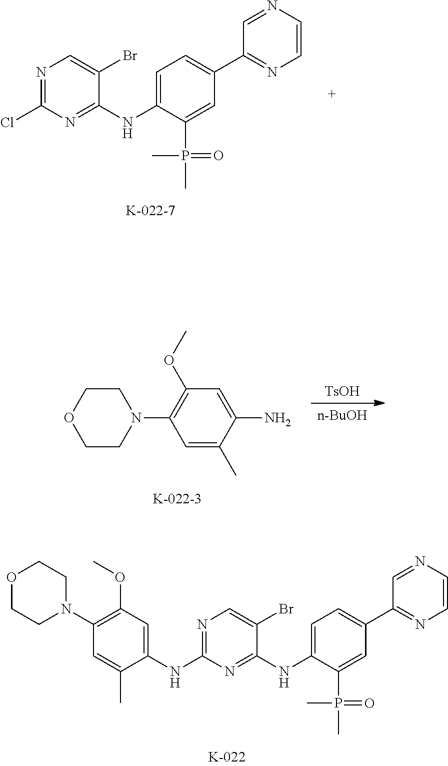

The compound K-022 was synthesized as the method described for compound K-018 using compound K-022-3 instead of raw compound K-018-9, compound K-022-7 instead of raw compound K-018-5. For compound K-022, MS: 624.14 [M+H]⁺

### Example 36: Synthesis of Compound K-017

### (2-((5-Bromo-2-((5-methoxy-2-methyl-4-(4-(4-methylpiperazin-1-yl)piperidin-1-yl)phenyl)amino)pyrimidin-4-yl)amino)-5-(pyrazin-2-yl)phenyl)dimethylphosphine oxide

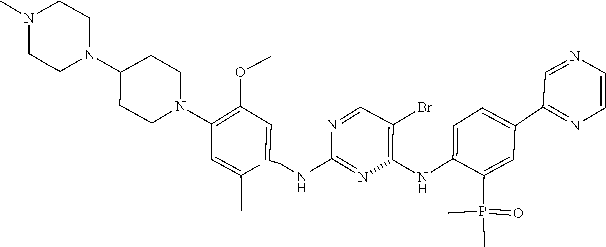

### Step 1: Synthesis of Compound K-017

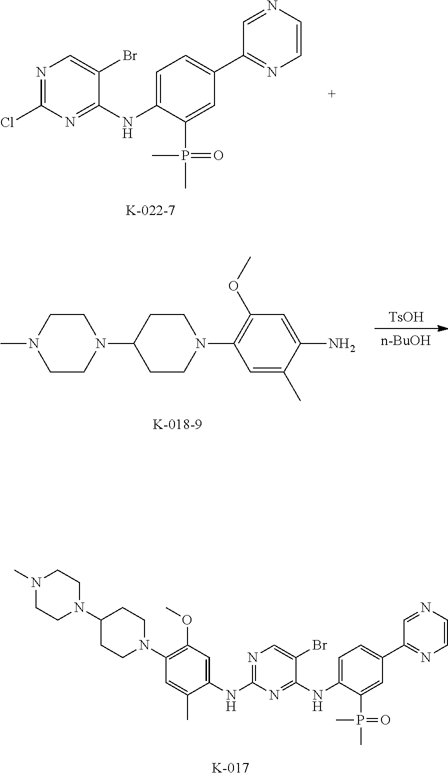

The compound K-017 was synthesized as the method described for compound K-018 using compound K-022-7 instead of raw compound K-018-5. For compound K-017, MS: 720.25 [M+H]⁺

### Example 37: Synthesis of Compound K-005

### (2-((5-Bromo-2-((5-(1-ethyl-1H-pyrazol-4-yl)-2-methoxy-4-(4-(4-methylpiperazin-1-yl)piperidin-1-yl)phenyl)amino)pyrimidin-4-yl)amino)-5-(1,5-dimethyl-1H-pyrazol-3-yl)phenyl)dimethylphosphine oxide

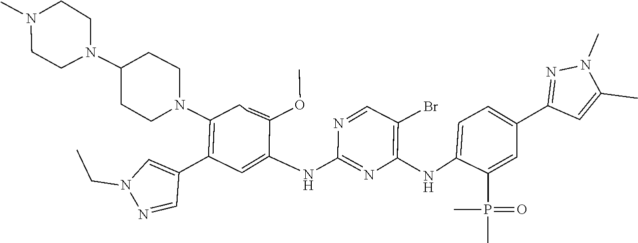

### Step 1: Synthesis of Compound K-005-1

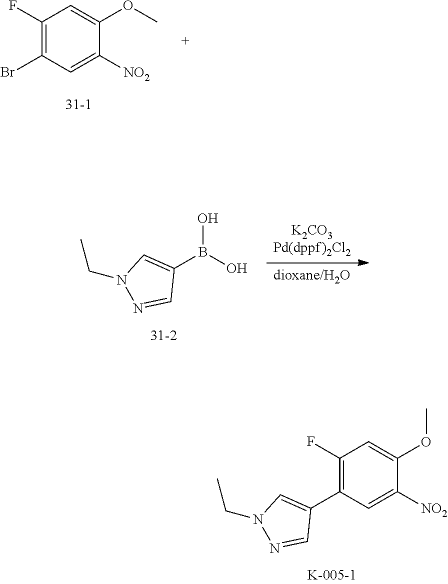

The compound K-005-1 was synthesized as the method described for compound K-022-6 using compound 31-2 instead of raw compound K-022-4, compound 31-1 instead of raw compound K-022-5. For compound K-005-1, MS: 226.09 [M+H]⁺

### Step 2: Synthesis of Compound K-005-2

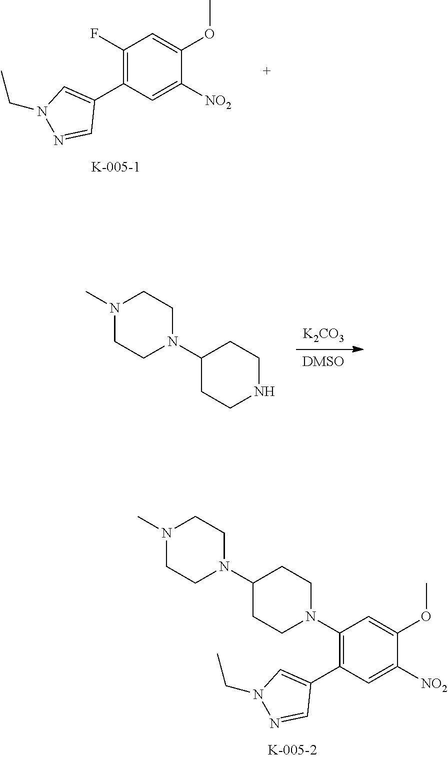

The compound K-005-2 was synthesized as the method described for compound K-018-8 using compound K-005-1 instead of raw compound K-018-6, compound 1-Methyl-4-(piperidin-4-yl)piperazine instead of raw compound K-018-7. For compound K-005-1, MS: 429.25 [M+H]⁺

### Step 3: Synthesis of Compound K-005-3

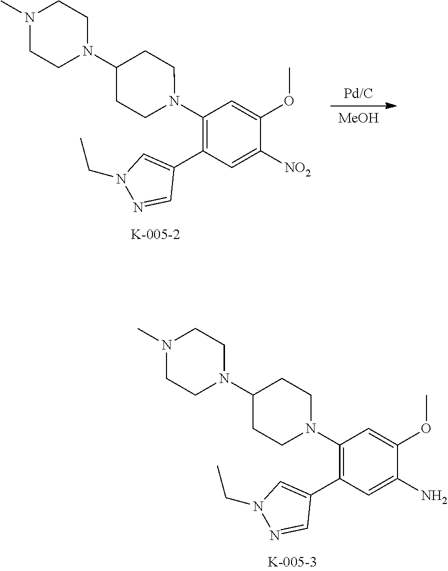

The compound K-005-3 was synthesized as the method described for compound K-018-9 using compound K-005-2 instead of raw compound K-018-8. For compound K-005-3, MS: 399.28 [M+H]⁺

### Step 4: Synthesis of Compound K-005

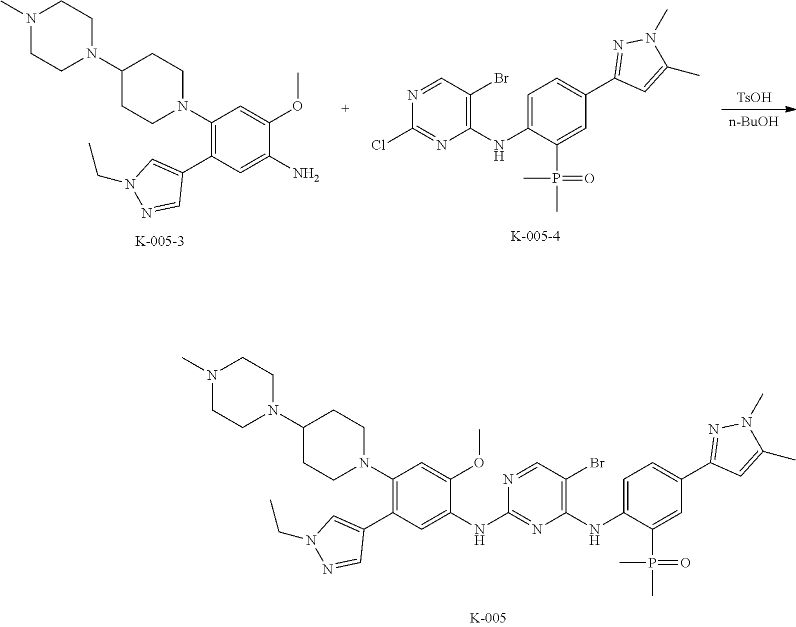

The compound K-005 was synthesized as the method described for compound K-018 using compound K-005-3 instead of raw compound K-018-9, compound K-005-4 instead of raw compound K-018-5. For compound K-005, MS: 816.31 [M+H]⁺

### Example 38: Synthesis of Compound K-016

### (2-((5-Bromo-2-((2-methoxy-5-methyl-4-(4-(4-methylpiperazin-1-yl)piperidin-1-yl)phenyl)amino)pyrimidin-4-yl)amino)-5-(5-ethynylthiophen-2-yl)phenyl)dimethylphosphine oxide

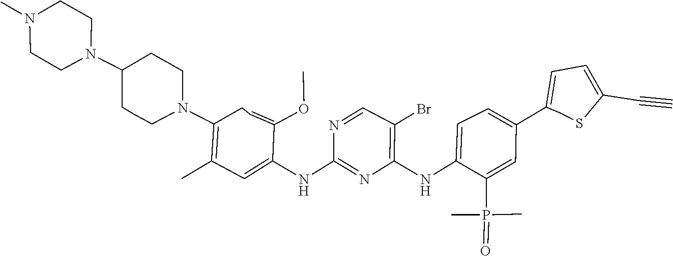 [SMILES: C#CC1=CC=C(c2ccc(Nc3nc(NC4=CC(C)=C(N5CCCC(N6CCN(C)CC6)CC5)CC=C4OC)ncc3Br)c(P(C)(C)=O)c2)CS1]

### Step 1: Synthesis of Compound K-016-1

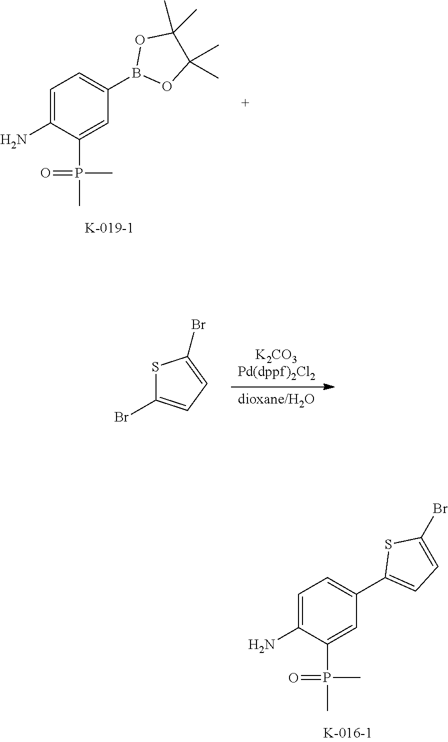 [SMILES: CC1(C)OB(c2ccc(N)c(P(C)(C)=O)c2)OC1(C)C ; Brc1ccc(Br)s1 ; "K2CO3Pd(dppf)2Cl2" ; "dioxane/H2O" ; CP(C)(=O)c1cc(-c2ccc(Br)s2)ccc1N]

The compound K-016-1 was synthesized as the method described for compound K-019-2 using 2,5-Dibromothiophene instead of 2-Bromothiazole. For compound K-016-1, MS: 329.96 [M+H]⁺

### Step 2: Synthesis of Compound K-016-2

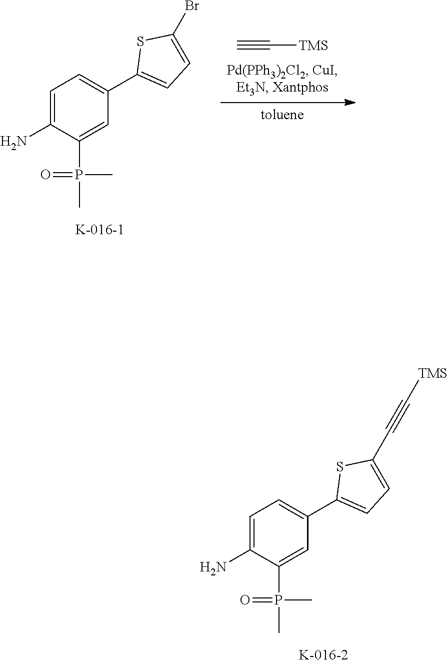

The compound K-016-2 was synthesized as the method described for compound K-013-3 using compound K-016-1 instead of compound K-013-2. For compound K-016-2, MS: 348.09 [M+H]⁺

### Step 3: Synthesis of Compound K-016-3

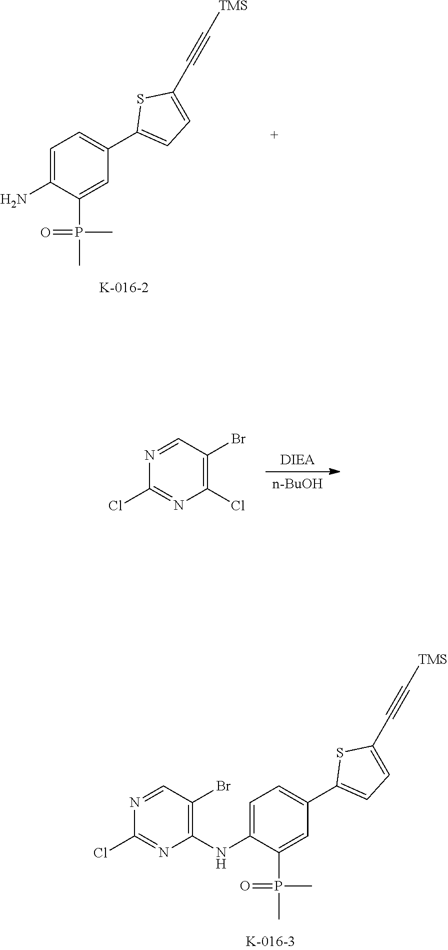

The compound K-016-3 was synthesized as the method described for compound K-019-3 using compound K-016-2 instead of raw compound K-019-2. For compound K-016-3, MS: 537.99 [M+H]⁺

### Step 4: Synthesis of Compound K-016-4

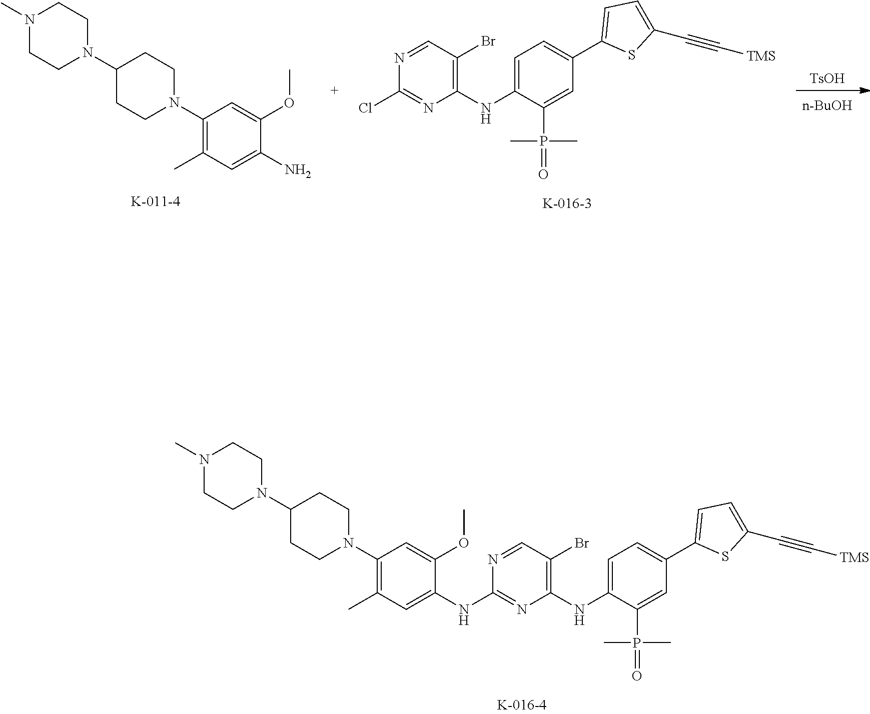

The compound K-016-4 was synthesized as the method described for compound K-019 using compound K-011-4 instead of raw compound K-018-9, compound K-016-3 instead of raw compound K-019-3. For compound K-016-4, MS: 820.25 [M+H]⁺

### Step 5: Synthesis of Compound K-016

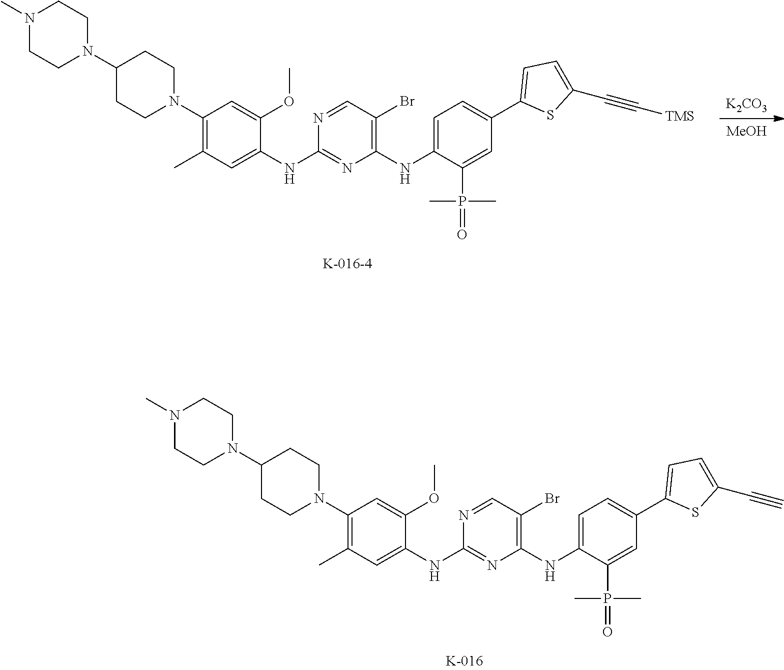

The compound K-016 was synthesized as the method described for compound K-010 using compound K-016-4 instead of raw compound K-010-6. For compound K-016 synthesized with general methods, MS: 748.21 [M+H]⁺

### Example 39: Synthesis of Compound K-002

### (2-((5-Bromo-2-((5-ethyl-2-methoxy-4-(4-(4-methylpiperazin-1-yl)piperidin-1-yl)phenyl)amino)pyrimidin-4-yl)amino)-5-(4-fluoro-1H-pyrazol-1-yl)phenyl)dimethylphosphine oxide

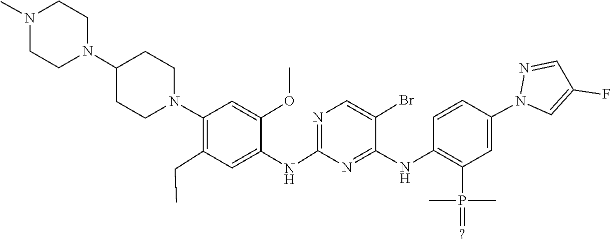

### Step 1: Synthesis of Compound K-002-1

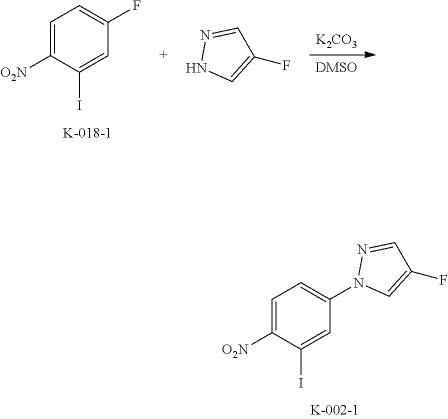

The compound K-002-1 was synthesized as the method described for compound K-018-2 using compound 4-Fluoro-1H-pyrazole instead of raw compound K-018-1. For compound K-002-1, MS: 334 [M+H]⁺

### Step 2: Synthesis of Compound K-002-2

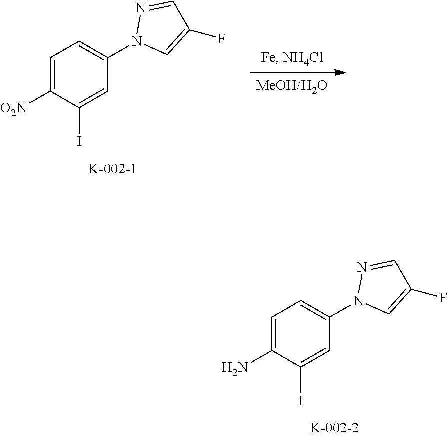

The compound K-002-2 was synthesized as the method described for compound K-018-3 using compound K-002-1 instead of raw compound K-018-2. For compound K-002-2, MS: 304 [M+H]⁺

### Step 3: Synthesis of Compound K-002-3

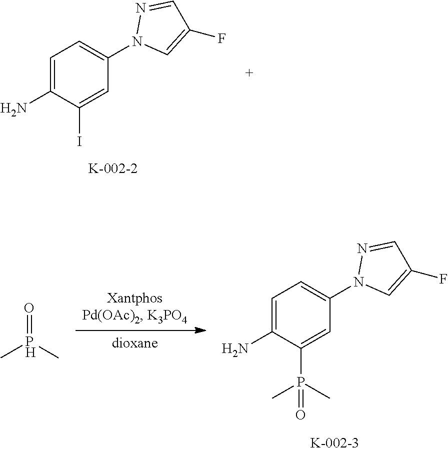

The compound K-002-3 was synthesized as the method described for compound K-018-4 using compound K-002-2 instead of raw compound K-018-3. For compound K-002-3, MS: 254 [M+H]⁺

### Step 4: Synthesis of Compound K-002-4

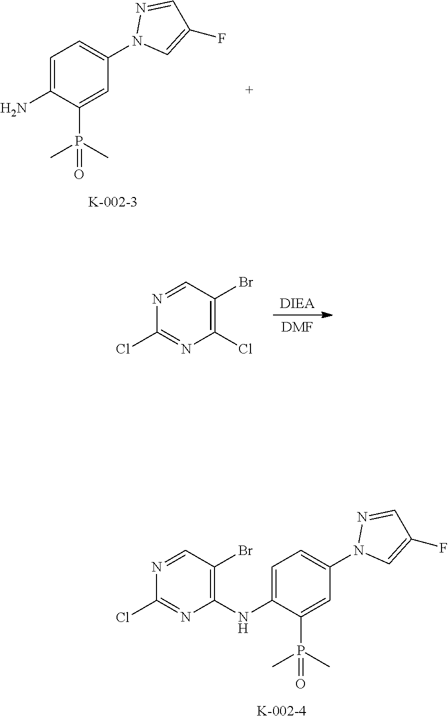

The compound K-002-4 was synthesized as the method described for compound K-018-5 using compound K-002-3 instead of raw compound K-018-4. For compound K-002-4, MS: 443.97 [M+H]⁺

### Step 5: Synthesis of Compound K-002

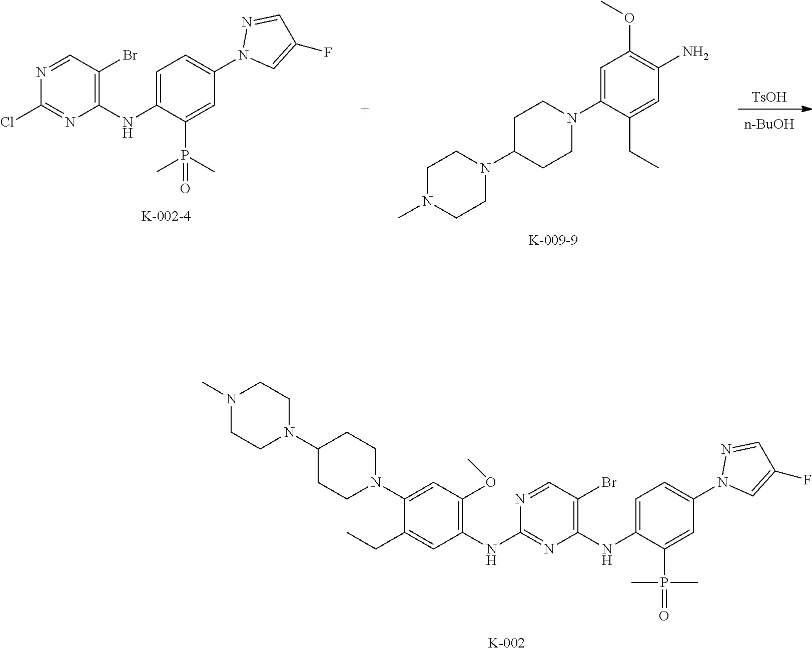

Add compound K-002-4 (100 mg), K-009-9 (75 mg), p-toluenesulfonic acid (58 mg) and n-butanol (2 mL) to the reaction flask, heat to 120° C., and stir for 12 hr. The reaction was complete as monitored by LCMS, and then stopped. The reaction solution was poured into sodium carbonate water (5 mL) solution, and extracted with dichloromethane (3×10 mL); the organic phase was washed with saturated brine (3×10 mL), dried over anhydrous sodium sulfate, separated and purified by column chromatography (chloromethane:methanol=12:1), and then concentrated to obtain the off-white solid compound K-002 (70 mg), MS: 740.25 [M+H]⁴

### Example 40: Synthesis of Compound K-041

### (6-((5-Bromo-2-((5-ethyl-2-methoxy-4-(4-(4-methylpiperazin-1-yl)piperidin-1-yl)phenyl)amino)pyrimidin-4-yl)amino)-2-methoxy-3-(1-methyl-1H-pyrazol-4-yl)phenyl)dimethylphosphine oxide

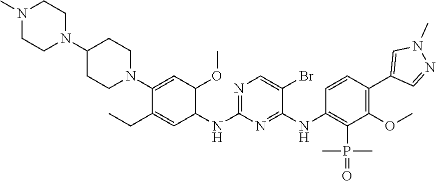 [SMILES: CCC1=CC(Nc2ncc(Br)c(Nc3ccc(-c4cnn(C)c4)c(OC)c3P(C)(C)=O)n2)C(OC)C=C1N1CCC(N2CCN(C)CC2)CC1]

### Step 1: Synthesis of Compound K-041-1

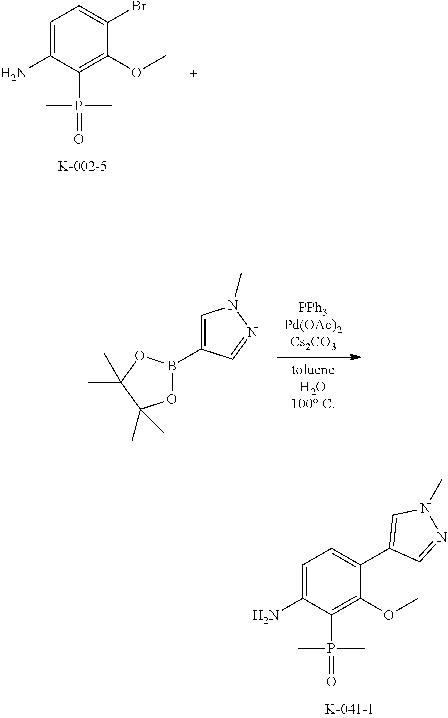 [SMILES: COc1c(Br)ccc(N)c1P(C)(C)=O ; Cn1cc(B2OC(C)(C)C(C)(C)O2)cn1 ; "tolueneH2O100° C." ; COc1c(-c2cnn(C)c2)ccc(N)c1P(C)(C)=O ; "PPh3Pd(OAc)2Cs2CO3"]

The compound K-041-1 was synthesized as the method described for compound K-019-1 using compound 1-methylpyrazol-4-boronic acid pinacol ester instead of raw compound bis(pinacolato)diboron. For compound K-041-1, MS: 280.11 [M+H]⁺

### Step 2: Synthesis of Compound K-041-2

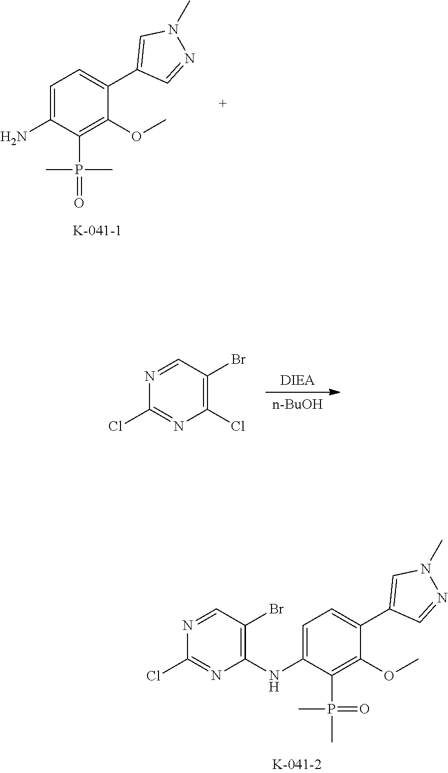

The compound K-041-2 was synthesized as the method described for compound K-019-3 using compound K-041-1 instead of raw compound K-019-2. For compound K-041-2, MS: 470.01 [M+H]⁺

### Step 3: Synthesis of Compound K-041

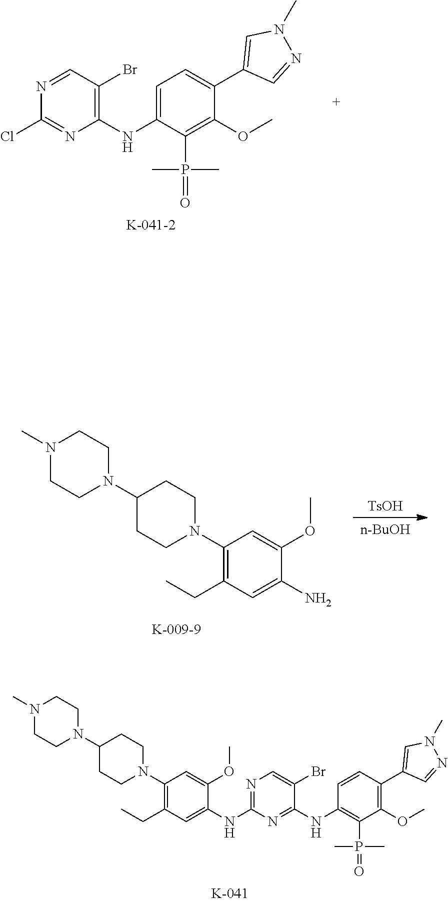

The compound K-041 was synthesized as the method described for compound K-019 using compound K-009-9 instead of raw compound K-018-9, compound K-041-2 instead of raw compound K-019-3. For compound K-041, MS: 766.29 [M+H]⁺

### Example 41: Synthesis of Compound

### (2-((5-Bromo-2-((5-ethyl-2-methoxy-4-(4-(pyrrolidin-1-yl)piperidin-1-yl)phenyl)aminopyrimidin-4-yl)amino)-5-(1-methyl-1-1H-pyrazol-4-yl)phenyl)dimethylphosphine oxide

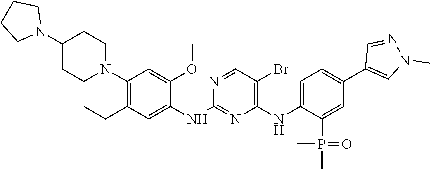 [SMILES: CCc1cc(Nc2ncc(Br)c(Nc3ccc(-c4cnn(C)c4)cc3P(C)(C)=O)n2)c(OC)cc1N1CCC(N2CCCC2)CC1]

### Step 1: Synthesis of Compound K-001-1

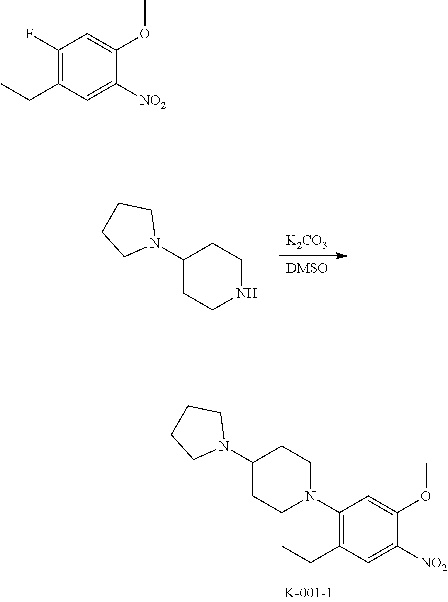

The compound K-001 was synthesized as the method described for compound K-018-8 using compound 1-Ethyl-2-fluoro-4-methoxy-5-nitrobenzene instead of raw compound K-018-6, compound 4-(1-Pyrrolidine)piperidine instead of raw compound K-018-7. For compound K-001-1, MS: 334.21 [M+H]⁺

### Step 2: Synthesis of Compound K-001-2

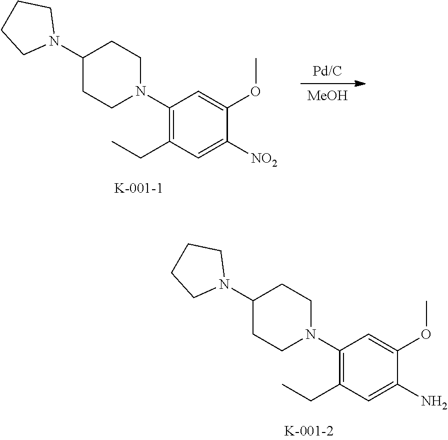 [SMILES: "MeOH" ; CCc1cc([N+](=O)[O-])c(OC)cc1N1CCC(N2CCCC2)CC1 ; "Pd/C" ; CCc1cc(N)c(OC)cc1N1CCC(N2CCCC2)CC1]

The compound K-001-2 was synthesized as the method described for compound K-018-9 using compound K-001-1 instead of raw compound K-018-8. For compound K-001-2, MS: 304.23 [M+H]⁺

### Step 3: Synthesis of Compound K-001

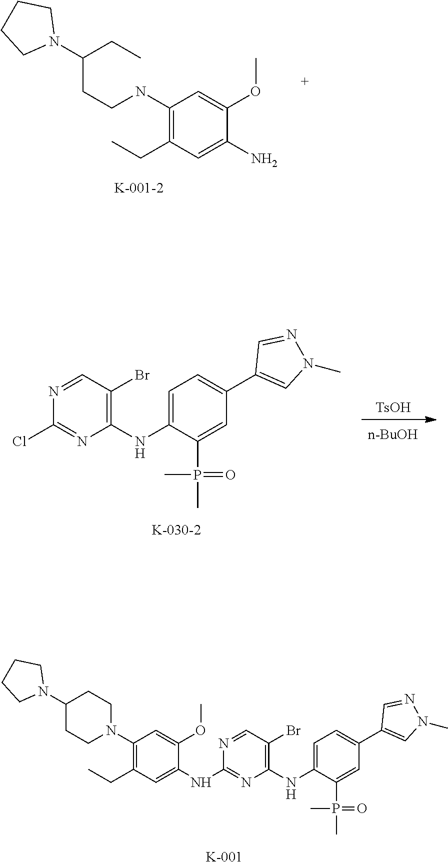

The compound K-001 was synthesized as the method described for compound K-019 using compound K-001-2 instead of raw compound K-018-9, compound K-030-2 instead of raw compound K-019-3. For compound K-001, MS: 707.25 [M+H]⁺

### Example 42: Synthesis of Compound K-032

### (2-((5-Bromo-2-((5-methoxy-2-methyl-4-morpholinophenyl)amino)pyrimidin-4-yl)amino)-5-(5-methylpyridin-2-yl)phenyl)dimethylphosphine oxide

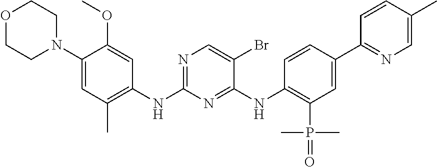 [SMILES: COc1cc(Nc2ncc(Br)c(Nc3ccc(-c4ccc(C)cn4)cc3P(C)(C)=O)n2)c(C)cc1N1CCOCC1]

### Step 1: Synthesis of Compound K-032-2

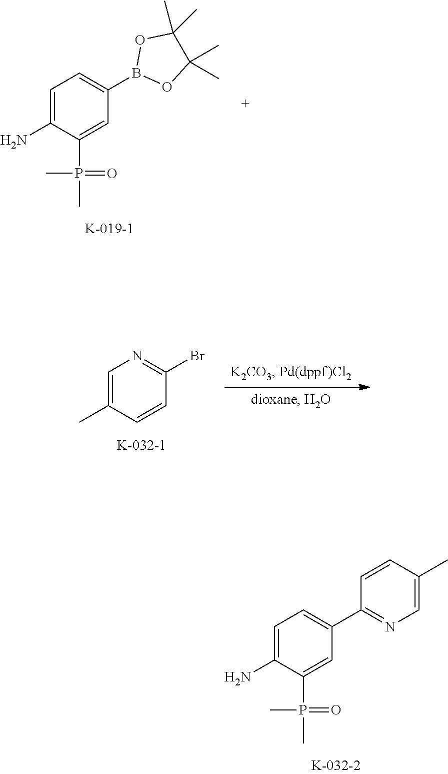

The compound K-032-2 was synthesized as the method described for compound K-022-6 using compound K-032-1 instead of raw compound K-022-5. For compound K-032-2, MS: 261.11 [M+H]⁺

### Step 2: Synthesis of Compound K-032-3

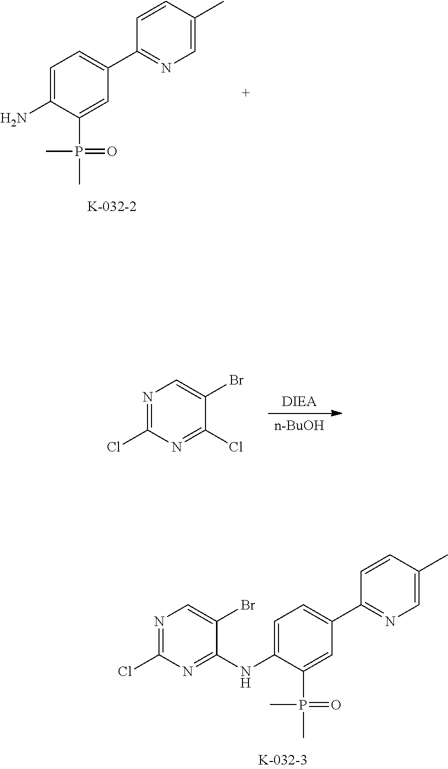

The compound K-032-3 was synthesized as the method described for compound K-004-5 using compound K-032-2 instead of raw compound K-004-4. For compound K-032-3, MS: 451.00 [M+H]⁺

### Step 3: Synthesis of Compound K-032

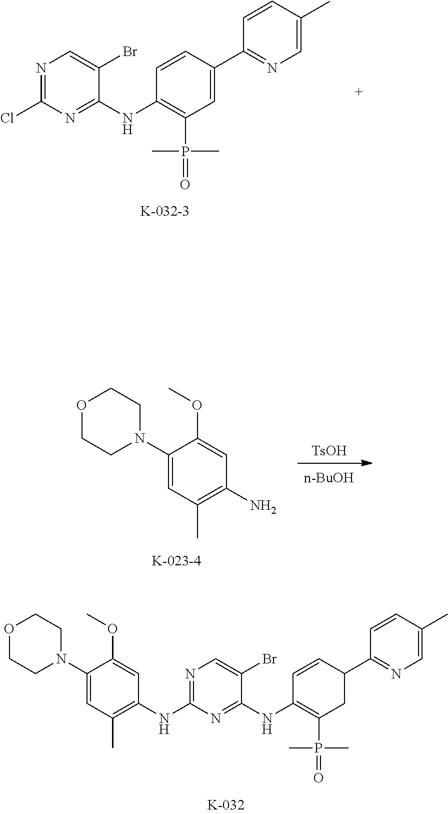

The compound K-032 was synthesized as the method described for compound K-018 using compound K-032-3 instead of raw compound K-018-5, compound K-023-4 instead of raw compound K-018-9. For compound K-032, MS: 637.16 [M+H]⁺

### Example 43: Synthesis of Compound K-035

### (2-((5-Bromo-2-((5-ethoxy-2-methyl-4-morpholinophenyl)amino)pyrimidin-4-yl)amino)-5-(5-methylpyridin-2-yl)phenyl)dimethylphosphine oxide

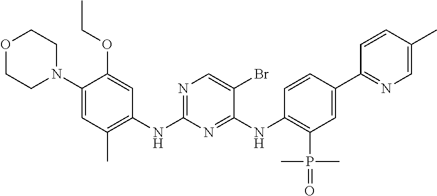 [SMILES: CCOc1cc(Nc2ncc(Br)c(Nc3ccc(-c4ccc(C)cn4)cc3P(C)(C)=O)n2)c(C)cc1N1CCOCC1]

### Step 1: Synthesis of Compound K-035-2

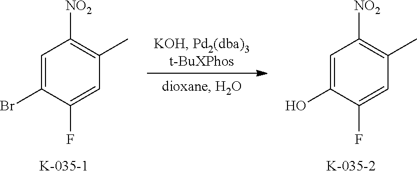

Add K-035-1 (10 g), KOH (3.6 g), t-BuXPhos (1.0 g), Pd₂(dba)₃ (1.97 g), 1,4-Dioxane (100 mL) and H₂O (10 mL) to the reaction flask, raise temperature to 100° C. and react for 3 hr under nitrogen protection. Lower the temperature, add 500 mL of DCM, adjust pH value of the solution to be acidic with 4N HCl; the layers were separated, and the organic phase was concentrated and purified by column chromatography (ethyl acetate:petroleum ether=3:1) to obtain compound K-035-2 (1.3 g), MS: 172.03 [M+H]⁺

### Step 2: Synthesis of Compound K-035-3

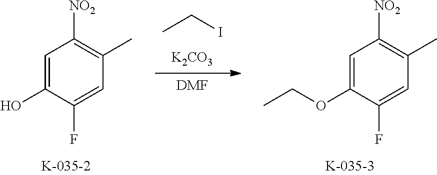

Add compound K-035-2 (300 mg), iodoethane (547 mg), potassium carbonate (484 mg) and DMF (5 mL) to the reaction flask, raise temperature to 50° C. and react for 6 hr. Lower the temperature, add 20 ml, of DCM for washing, saturated brine for washing; the organic phase was dried, concentrated and purified by column chromatography (ethyl acetate:petroleum ether-5:1) to obtain compound K-035-3 (140 mg).

### Step 3: Synthesis of Compound K-035-3

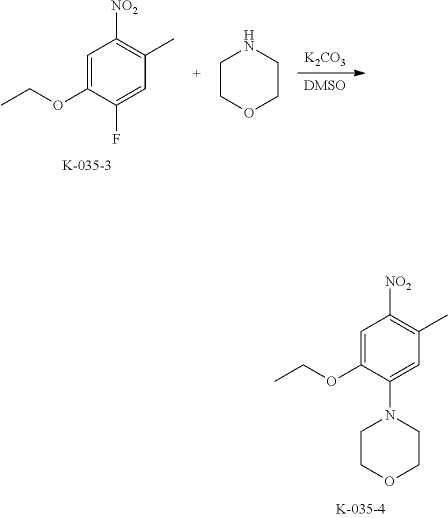

The compound K-Uh-q was synthesized as the method described for compound K-024-1 using morpholine and K-035-3 instead of two raw compounds. For compound K-035-4, MS: 267.13 [M+H]⁺

### Step 2: Synthesis of Compound K-035-5

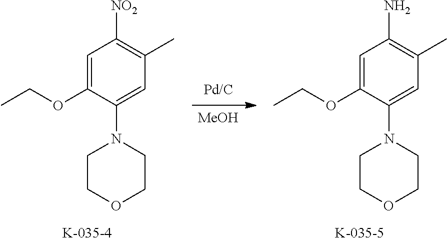 [SMILES: "MeOH" ; CCOc1cc([N+](=O)[O-])c(C)cc1N1CCOCC1 ; "Pd/C" ; CCOc1cc(N)c(C)cc1N1CCOCC1]

The compound K-035-5 was synthesized as the method described for compound K-024-2 using compound K-035-4 instead of raw compound K-024-1. For compound K-035-5, MS: 237.15 [M+H]⁺

### Step 3: Synthesis of Compound K-035

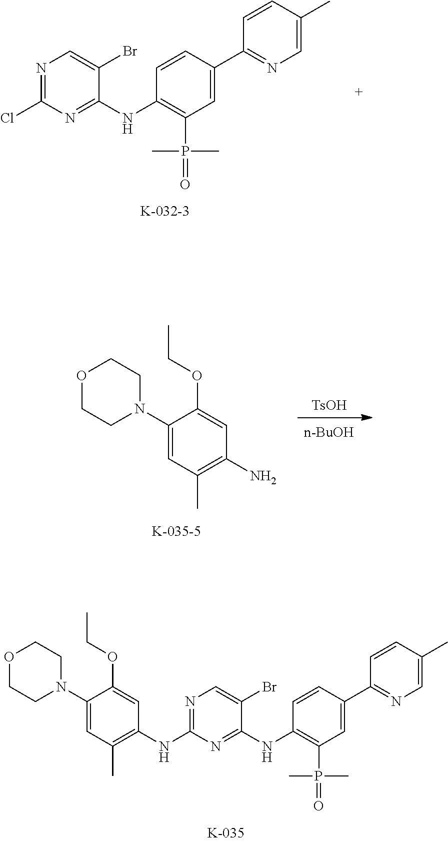

The compound K-035 was synthesized as the method described for compound K-018 using compound K-032-3 instead of raw compound K-018-5, compound K-035-5 instead of raw compound K-018-9. For compound K-035, MS: 651.18 [M+H]⁺

### Example 44: Synthesis of Compound K-034

### (6-((5-Bromo-2-((5-ethoxy-2-methyl-4-morpholinophenyl)amino)pyrimidin-4-yl)amino)-2-fluoro-3-(2-methyl-2H-1,2,3-triazol-4-yl)phenyl)dimethylphosphine oxide

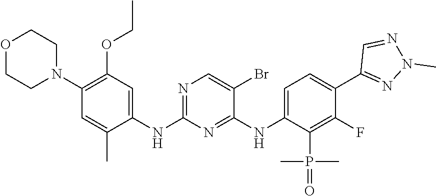 [SMILES: CCOc1cc(Nc2ncc(Br)c(Nc3ccc(-c4cnn(C)n4)c(F)c3P(C)(C)=O)n2)c(C)cc1N1CCOCC1]

### Step 1: Synthesis of Compound K-034

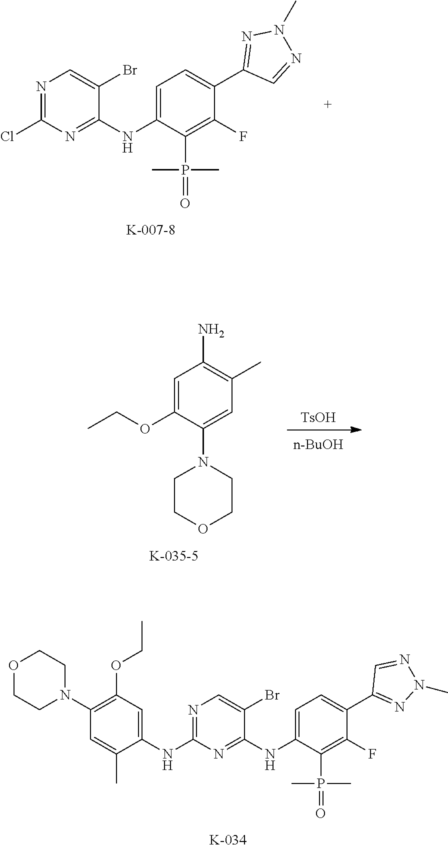

The compound K-007 was synthesized as the method described for compound K-018 using compound K-035-5 instead of raw compound K-018-9, compound K-007-8 instead of raw compound K-018-5. For compound K-034, MS: 659.16 [M+H]⁺

¹H NMR (500 MHz, DMSO-d6) δ 11.91 (s, 1H), 8.81 (s, 1H), 8.35 (s, 1H), 8.19 (s, 1H), 7.96 (d, J=3.9 Hz, 1H), 7.60 (s, 1H), 6.84 (s, 1H), 6.79 (s, 1H), 4.21 (s, 3H), 3.90 (q, J=6.9 Hz, 2H), 3.78 (t, J=4.5 Hz, 4H), 3.06 (t, J=4.6 Hz, 4H), 2.07 (s, 3H), 1.89 (dd, J=13.9, 2.3 Hz, 6H), 1.24 (d, J=7.0 Hz, 3H).

### Example 45: Synthesis of Compound K-036

### (6-((5-Bromo-2-((2-methyl-4-morpholin-5-(2,2,2-trifluoroethoxy)phenyl)amino)pyrimidin-4-yl)amino)-2-fluoro-3-(2-methyl-2H-1,2,3-triazol-4-yl)phenyl)dimethylphosphine oxide

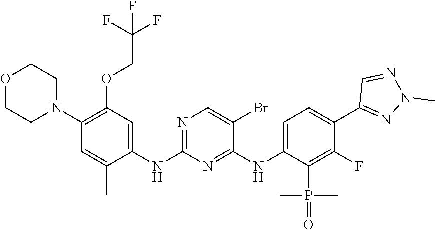 [SMILES: Cc1cc(N2CCOCC2)c(OCC(F)(F)F)cc1Nc1ncc(Br)c(Nc2ccc(-c3cnn(C)n3)c(F)c2P(C)(C)=O)n1]

### Step 1: Synthesis of Compound K-036-1

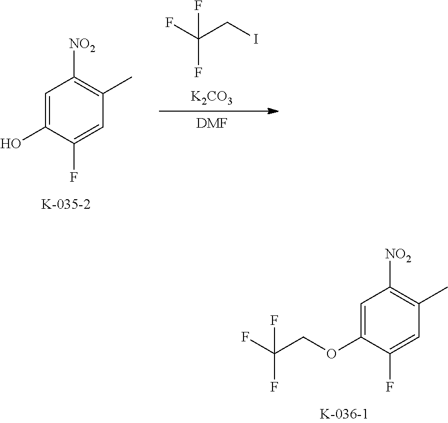

The compound K-036-1 was synthesized as the method described for compound K-035-3 using compound 1,1,1-Trifluoro-2-iodoethane instead of raw compound iodoethane.

### Step 2: Synthesis of Compound K-036-2

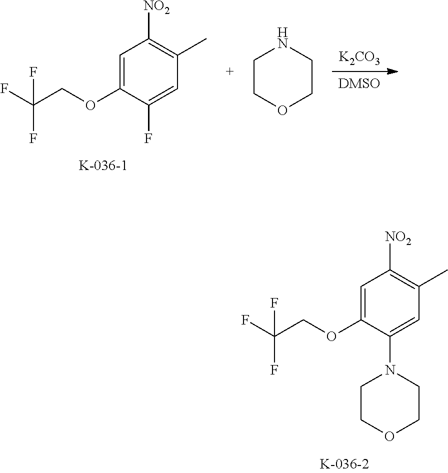

The compound K-036-2 was synthesized as the method described for compound K-035-4 using compound K-036-1 instead of raw compound K-035-3. For compound K-036-2, MS: 321.10 [M+H]⁺

### Step 2: Synthesis of Compound K-036-3

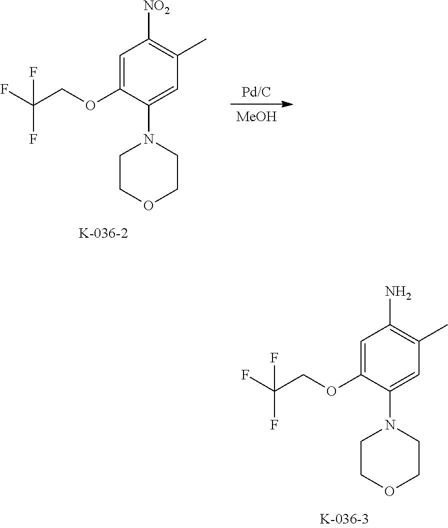 [SMILES: "MeOH" ; Cc1cc(N2CCOCC2)c(OCC(F)(F)F)cc1[N+](=O)[O-] ; "Pd/C" ; Cc1cc(N2CCOCC2)c(OCC(F)(F)F)cc1N]

The compound K-036-3 was synthesized as the method described for compound K-035-5 using compound K-036-2 instead of raw compound K-035-4. For compound K-036-3, MS: 291.12 [M+H]⁺

### Step 3: Synthesis of Compound K-036

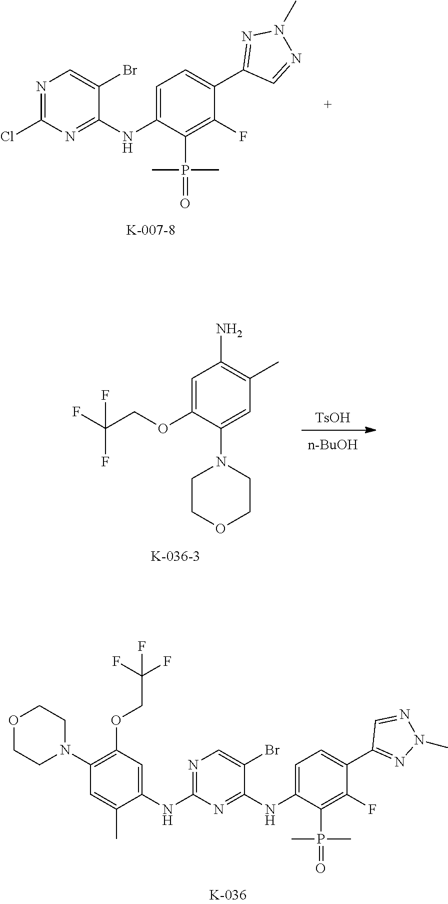

The compound K-036 was synthesized as the method described for compound K-034 using compound K-036-3 instead of raw compound K-035-5. For compound K-036, MS: 713.13 [M+H]⁺

### Example 46: Synthesis of Compound K-046

### (6-((5-Bromo-2-((5-ethyl-2-methoxy-4-(4-(4-methylpiperazin-1-yl)piperidin-1-yl)phenyl)amino)pyrimidin-4-yl)amino)-2-methyl-3-(1-methyl-1H-pyrazol-4-yl)phenyl)dimethylphosphine oxide

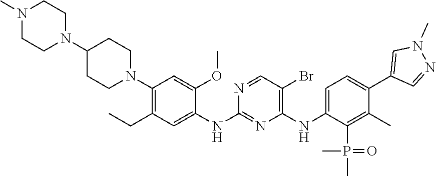 [SMILES: CCc1cc(Nc2ncc(Br)c(Nc3ccc(-c4cnn(C)c4)c(C)c3P(C)(C)=O)n2)c(OC)cc1N1CCC(N2CCN(C)CC2)CC1]

### Step 1: Synthesis of Compound K-046-3

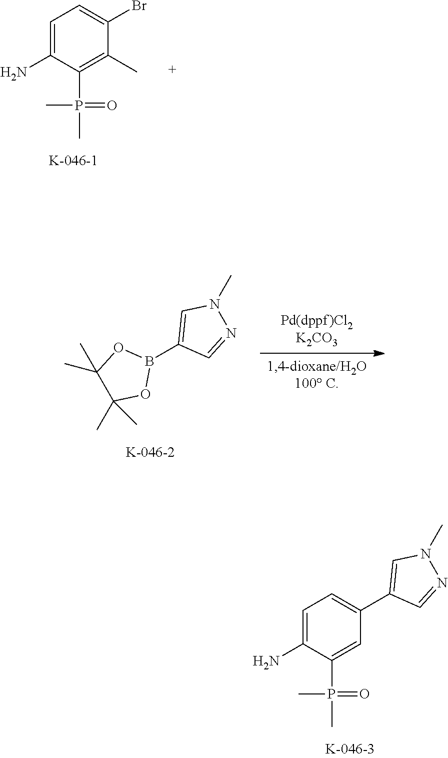

Dissolve compounds K-046-1 (500 mg), K-046-2 (476.33 mg), Pd(dppf)Cl₂ (156 mg) and anhydrous potassium carbonate (791 mg) to 1,4-dioxane (10 mL) and H₂O (2 mL), heat to 100° C. and stir overnight under nitrogen protection. The reaction resolution was cooled down to room temperature, concentrated, and purified by column chromatography (DCM/MeOH; MeOH: 0-5%). It was concentrated to obtain compound K-046-3, MS: 264.12 [M+H]⁺

### Step 2: Synthesis of Compound K-046-4

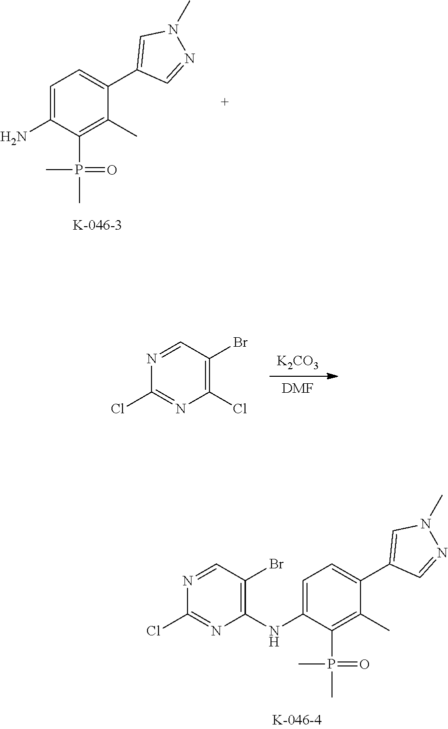

K-046-3 (300 mg) was dissolved to N,N-dimethylformamide (10 mL), 5-Bromo-2,4-dichloropyrimidine (779 mg) and anhydrous carbonate (709 mg) were added to the solution. The reaction solution was heated to 100° C. and stirred overnight. The reaction was complete as monitored by LCMS. The reaction solution was cooled down to room temperature, diluted with water (20 mL), extracted with ethyl acetate (20 mL) for three times; combined with organic phase, washed once with saturated sodium chloride (20 mL), dried, filtered and concentrated. The solution was purified by column chromatography (dichloromethane/methanol; methanol: 0-5%), and then concentrated to obtain the compound K-046-4, MS: 454.01 [M+H]⁺

### Step 3: Synthesis of Compound K-046

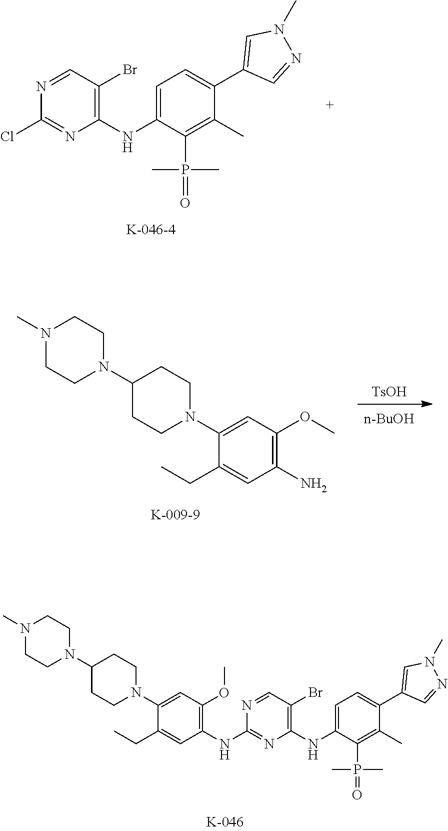

Dissolve compounds K-046-4 (100 mg), K-009-9 (88 mg) and p-toluenesulfonic acid (57 mg) to n-butanol (5 mL), heat to 100° C. and stir overnight. The reaction solution was cooled down to room temperature, concentrated, adjusted pH of 9-10 with saturated sodium carbonate solution, extracted with dichloromethane (20 mL×2), combined with organic phase, dried, filtered and concentrated. It was purified by preparative thin layer chromatography (DCM/MeOH-10/1) to obtain compound K-046, MS: 750.29 [M+H]⁺

### Example 47: Synthesis of Compound K-050

### (4-((5-Bromo-2-((5-ethyl-2-methoxy-4-(4-(4-methylpiperazin-1-yl)piperidin-1-yl)phenyl)amino)pyrimidin-4-yl)amino)-2-methyl-[1,1′-biphenyl]-3-yl)dimethylphosphine oxide

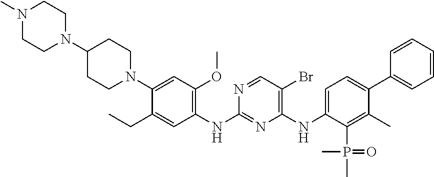 [SMILES: CCc1cc(Nc2ncc(Br)c(Nc3ccc(-c4ccccc4)c(C)c3P(C)(C)=O)n2)c(OC)cc1N1CCC(N2CCN(C)CC2)CC1]

### Step 1: Synthesis of Compound K-050-2

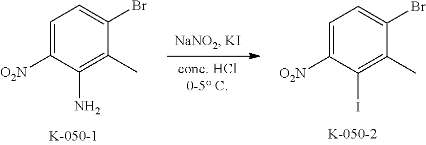

Dissolve K-050-1 (5.00 g) in concentrated hydrochloric acid (20 mL), cool down to 0-5° C. in an ice-salt bath, add dropwise an aqueous solution (20 mL) of sodium nitrite (1.79 g), after that, stir at −5° C. for 0.5 h, then add dropwise an aqueous solution (20 mL) of KI (5.39 g) at 0-5° C. and stir at room temperature overnight. Ethyl acetate (60 mL) was added to the reaction solution, and which was stirred for layering; the organic phase was collected, washed with water once, with aqueous sodium thiosulfate (20 mL×2) solution, saturated aqueous sodium chloride solution (20 mL), and dried, filtered and concentrated to obtain the compound K-050-2 (6.38 g).

### Step 2: Synthesis of Compound K-050-3

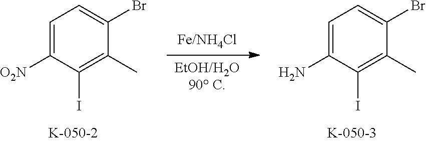

Dissolve K-050-2 (6.38 g) to anhydrous ethanol (100 mL) and water (20 mL), add iron powder (10.42 g) and anhydrous ammonium chloride (9.98 g), the reaction solution was heated to 90° C. and stirred for 2 hr. After complete reaction, the solution was cooled down to room temperature, and filtered with suction. The filter cake was rinsed with absolute ethanol; the filtrate was collected and concentrated. Add dichloromethane (100 mL), wash with water (2×20 mL), dry over anhydrous sodium sulfate, filter and concentrate to get the compound K-050-3 (5.42 g), MS: 311.88 [M+H]⁺

### Step 3: Synthesis of Compound K-050-4

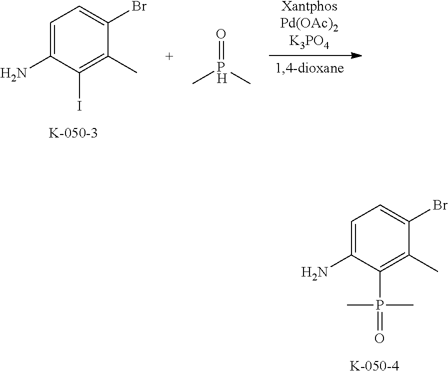

Dissolve compound K-050-3 (5.42 g) to 1, 4-dioxane (100 mL), add dimethylphosphine oxide (1.63 g). Xantphos (2.01 g), palladium acetate (391 mg) and anhydrous potassium phosphate (11.06 g). Heat to 100° C. under nitrogen and stir overnight. The reaction was complete as monitored by LCMS. The reaction solution was cooled down to room temperature, and filtered. The filtrate was collected, concentrated, separated and purified by column chromatography (DCM/MeOH; phase B: 0-5% of methanol), and then concentrated again to obtain the compound K-050-4 (3.56 g), MS: 261.99 [M+H]⁺

### Step 4: Synthesis of Compound K-050-5

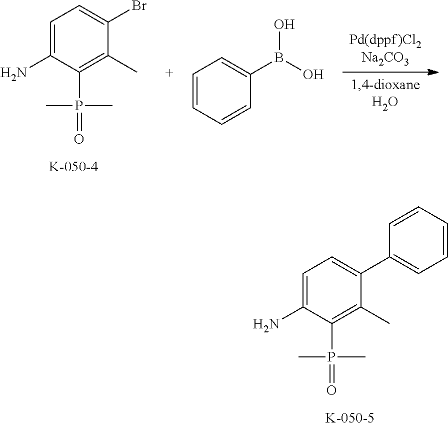 [SMILES: Cc1c(Br)ccc(N)c1P(C)(C)=O ; OB(O)c1ccccc1 ; "1,4-dioxaneH2O" ; "Pd(dppf)Cl2Na2CO3" ; Cc1c(-c2ccccc2)ccc(N)c1P(C)(C)=O]

Dissolve compound K-050-4 (500 mg) to 1,4-dioxane (10 mL) and water (2 mL), add phenylboronic acid (302 mg), Pd(dppf)Cl₂ (156 mg) and anhydrous sodium carbonate (607 mg). Heat to 100° C. under nitrogen and stir overnight. The reaction solution was cooled down to room temperature, concentrated under reduced pressure, separated and purified by column chromatography (phase A: dichloromethane; phase B: 0-5% of methanol), and then concentrated to obtain the compound K-050-5 (389 mg), MS: 260.11 [M+H]⁺

### Step 5: Synthesis of Compound K-050-6

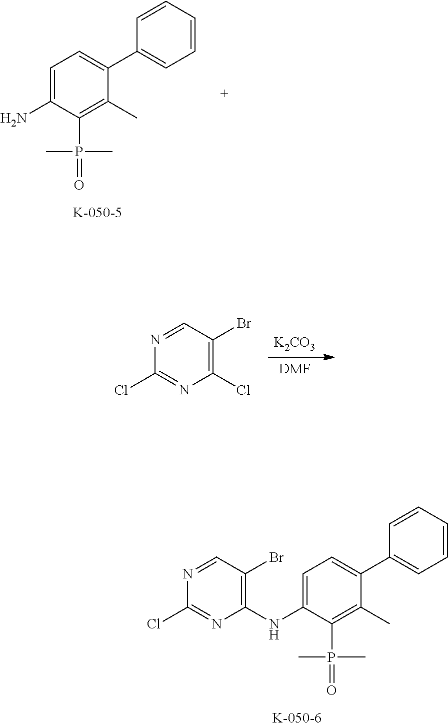

The compound K-050-6 was synthesized as the method described for compound K-046-4 using compound K-050-5 instead of raw compound K-046-3. For compound K-050-6, MS: 450.01 [M+H]⁺

### Step 6: Synthesis of Compound K-050

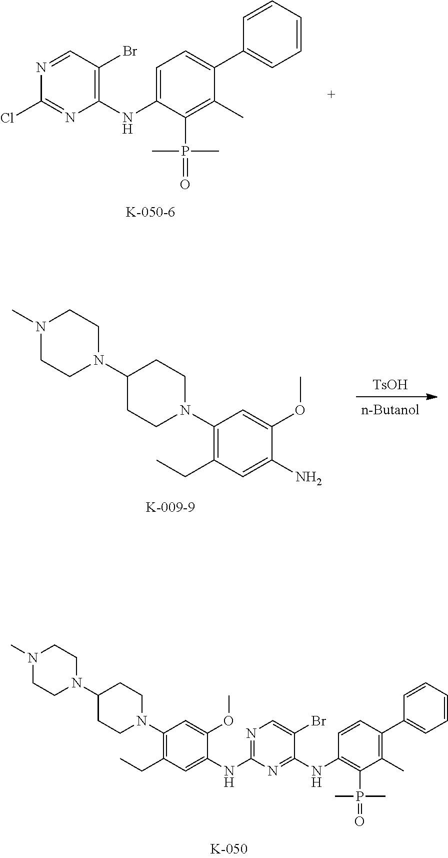

The compound K-050 was synthesized as the method described for compound K-046 using compound K-050-6 instead of raw compound K-046-4. For compound K-050, MS: 746.29 [M+H]⁺

### Example 48: Synthesis of Compound K-051

### (4-((2-((4-([1,4′-bipiperidin]-1′-yl)-2-methoxy-5-(1-methyl-1H-pyrazole-4-yl)phenyl)amino)-5-bromopyrimidin-4-yl)amino)-2-methyl-[(1,1′-biphenyl]-3-yl)dimethylphosphine oxide

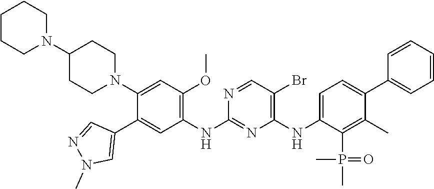 [SMILES: COc1cc(N2CCC(N3CCCCC3)CC2)c(-c2cnn(C)c2)cc1Nc1ncc(Br)c(Nc2ccc(-c3ccccc3)c(C)c2P(C)(C)=O)n1]

### Step 1: Synthesis of Compound K-051-3

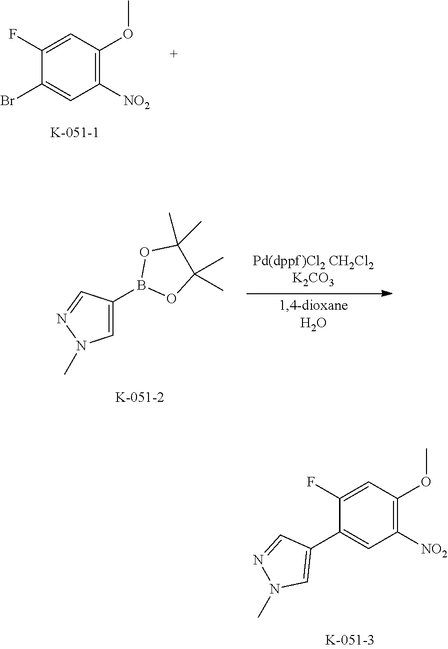 [SMILES: COc1cc(F)c(Br)cc1[N+](=O)[O-] ; Cn1cc(B2OC(C)(C)C(C)(C)O2)cn1 ; "1,4-dioxaneH2O" ; "Pd(dppf)Cl2 CH2Cl2K2CO3" ; COc1cc(F)c(-c2cnn(C)c2)cc1[N+](=O)[O-]]

Dissolve compounds K-051-1 (3.00 g) and K-051-2 (3.74 g) to 1,4-dioxane (50 mL) and water (10 mL), heat to 100° C. under nitrogen protection and stir overnight. The reaction solution was cooled down to room temperature, filtered, and purified by column chromatography (phase A: dichloromethane; phase B: 0-5% of methanol) to obtain the compound K-051-3 (2.20 g), MS: 252.07 [M+H]⁺

### Step 2: Synthesis of Compound K-051-5

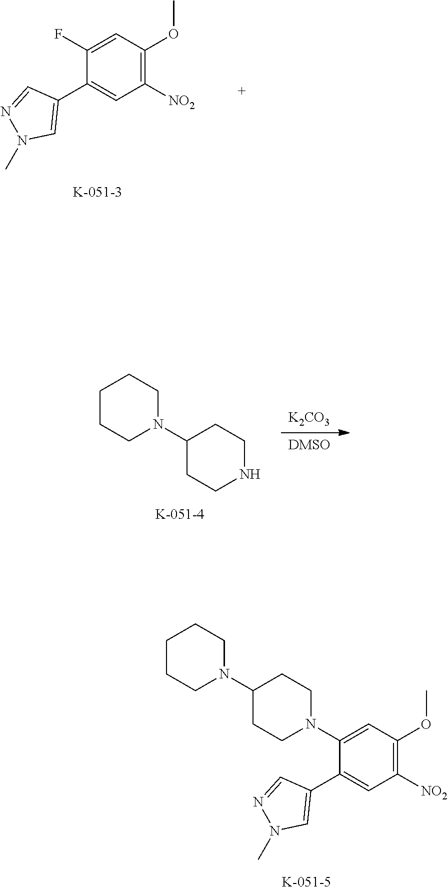

Dissolve compound K-051-3 (300 mg) in DMSO (10 mL), and add K-051-4 (301 mg) and anhydrous potassium carbonate (495 mg) to the solution. The reaction solution was heated to 90° C., stirred overnight and naturally cooled down to room temperature. After adding ethyl acetate (20 mL) and water (20 mL), the solution was extracted twice with ethyl acetate (20 mL×2), combined with the organic phase, washed with water (20 mL) and saturated sodium chloride solution (20 mL), dried, filtered and concentrated under reduced pressure to obtain the compound K-051-5 (240 mg), MS: 400.23 [M+H]⁺

### Step 3: Synthesis of Compound K-051-6

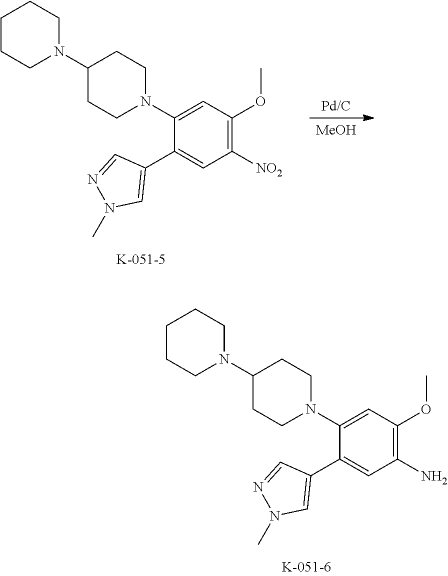 [SMILES: "MeOH" ; COc1cc(N2CCC(N3CCCCC3)CC2)c(-c2cnn(C)c2)cc1[N+](=O)[O-] ; "Pd/C" ; COc1cc(N2CCC(N3CCCCC3)CC2)c(-c2cnn(C)c2)cc1N]

Add compound K-051-5 (240 mg), Pd/C (50 mg) and absolute methanol (10 mL), and introduce H₂. The reaction solution was stirred at room temperature for 3 hr. The reaction was complete as monitored by LCMS, and then stopped. The solution was filtered with suction, and rinsed with methanol; the filtrate was collected and concentrated under reduced pressure to obtain desired product compound K-051-6 (200 mg), MS: 370.25 [M+H]⁺

### Step 4: Synthesis of Compound K-051

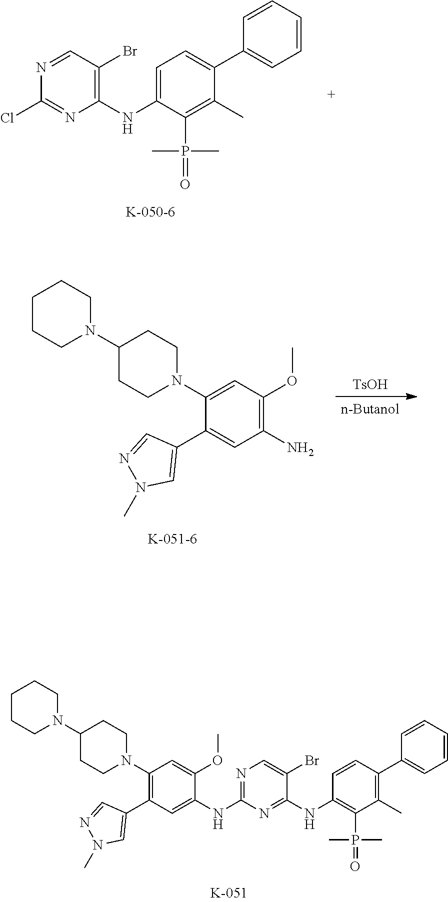 [SMILES: Cc1c(-c2ccccc2)ccc(Nc2nc(Cl)ncc2Br)c1P(C)(C)=O ; COc1cc(N2CCC(N3CCCCC3)CC2)c(-c2cnn(C)c2)cc1N ; "TsOH" ; "n-Butanol" ; COc1cc(N2CCC(N3CCCCC3)CC2)c(-c2cnn(C)c2)cc1Nc1ncc(Br)c(Nc2ccc(-c3ccccc3)c(C)c2P(C)(C)=O)n1]

The compound K-051 was synthesized as the method described for compound K-046 using compound K-050-6 instead of raw compound K-046-4, compound K-051-6 instead of raw compound K-046-5. For compound K-051, MS: 783.28 [M+H]⁺

### Example 49: Synthesis of Compound K-043

### (2-((5-Bromo-2-((2-methoxy-4-(4-(4-methylpiperazin-1-yl)piperidin-1-yl)-5-propylphenyl)amino)pyrimidin-4-yl)amino)-5-(1-methyl-1H-pyrazol-4-yl)phenyl)dimethylphosphine oxide

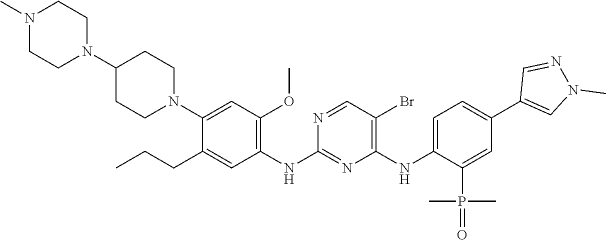

### Step 1: Synthesis of Compound K-043-3

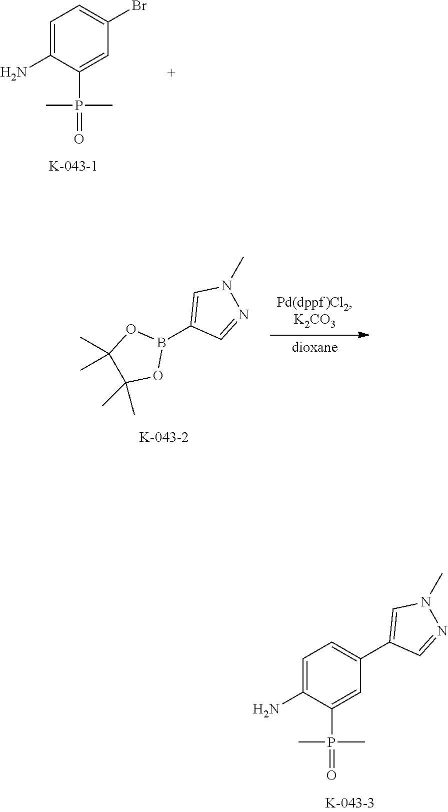

Add compounds K-043-1 (1.5 g). K-043-2 (1.51 g), Pd(dppf)Cl₂ (937 mg), K₂CO₃ (1.25 g), 1,4-dioxane (30 mL) and water (3 mL) to the reaction flask, heat to 1210° C. under nitrogen protection, and stir with heating for 16 hr. The reaction was complete as monitored by LCMS, and then stopped. The reaction solution was poured into water (50 mL), and extracted with dichloromethane (3×50 mL); the organic phase was washed with saturated brine (3×30 mL), dried over anhydrous sodium sulfate, separated and purified by column chromatography (dichloromethane:methanol=15:1), then concentrated and dried to obtain yellow solid of desired product compound K-043-3 (1.5 g), MS: 250.10 [M+H]⁺

### Step 2: Synthesis of Compound K-043-5

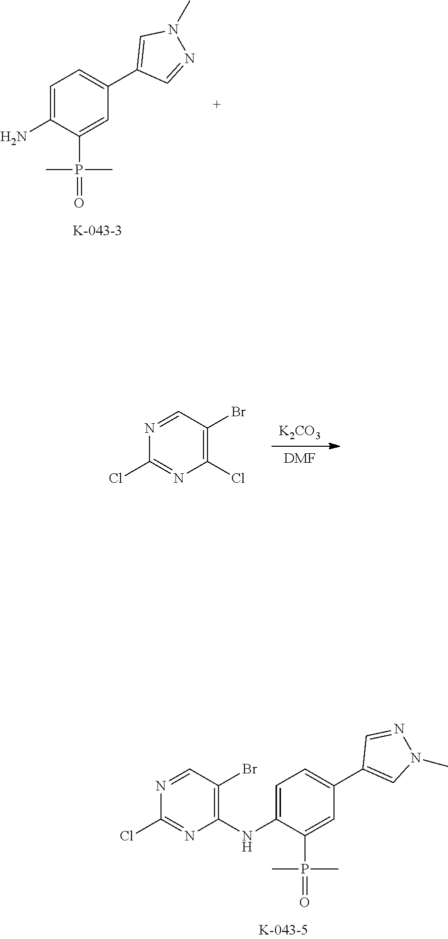

Dissolve K-043-3 (1.5 g), 5-Bromo-2,4-dichloropyrimidine (2.06 g) to DMF (20 mL), and add K₂CO₃ (1.66 g) at room temperature; and the gas in reactor was replaced with nitrogen for three times. After well stirring, the solution was transferred to an oil bath, heated to 100° C., and stirred at 100° C. for 16 hr. The reaction was complete as monitored by LCMS. The reaction solution was cooled down to room temperature, poured into water, and extracted with ethyl acetate (3×30 mL), dried over anhydrous sodium sulfate, separated and purified by column chromatography (dichloromethane:methanol=15:1) to obtain desired compound K-043-5 (790 mg), MS: 440.00 [M+H]⁺

### Step 3: Synthesis of Compound K-043-7

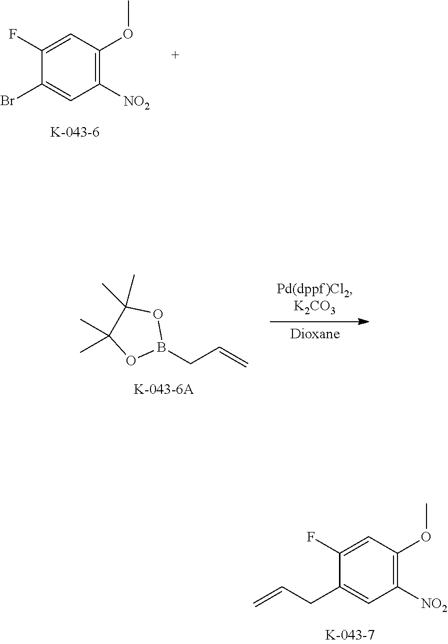

Add compounds K-043-6 (1.5 g), K-043-6A (2.02 g), Pd(dppf)Cl₂ (489 mg), K₂CO₃ (1.24 g), dioxane (30 ml) and water (3 mL) to the reaction flask, heat to 100° C. under nitrogen protection, and stir with heating for 1.5 hr. The reaction was complete as monitored by LCMS, and then stopped. The reaction solution was poured into water (50 mL), and extracted with dichloromethane (3×50 mL); the organic phase was washed with saturated brine (3×30 mL), dried over anhydrous sodium sulfate, separated and purified by column chromatography (N-hexane:ethyl acetate=2:1) to obtain the yellow solid of desired product K-043-7 (1.0 g), MS: 212.06 [M+H]⁺

### Step 4: Synthesis of Compound K-043-8

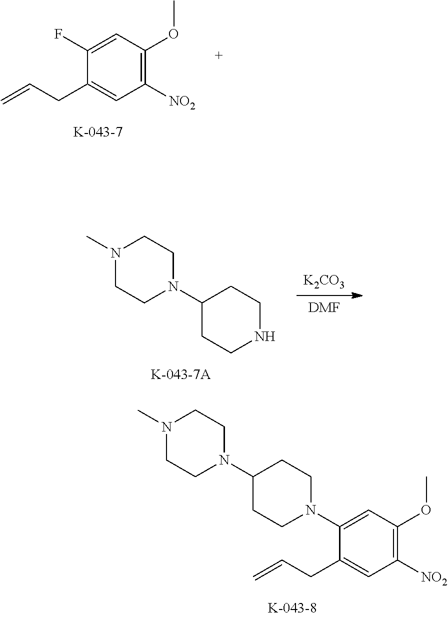

Dissolve K-043-7 (1.0 g) and K-043-7A (867 mg) in DMF (20 mL), add K₂CO₃ (1.31 g) to the solution at room temperature; the gas in reactor was replaced with nitrogen for three times; after well stirring, the solution was transferred to an oil bath, heated to 100° C., and stirred at 90° C. for 16 hr. The reaction was complete as monitored by LCMS. The reaction solution was cooled down to room temperature, poured into water, extracted with ethyl acetate (3×30 mL), dried over anhydrous sodium sulfate, separated and purified by column chromatography (dichloromethane:methanol=15:1) to obtain desired compound K-043-8 (400 mg), MS: 375.23 [M+H]⁺

### Step 5: Synthesis of Compound K-043-9

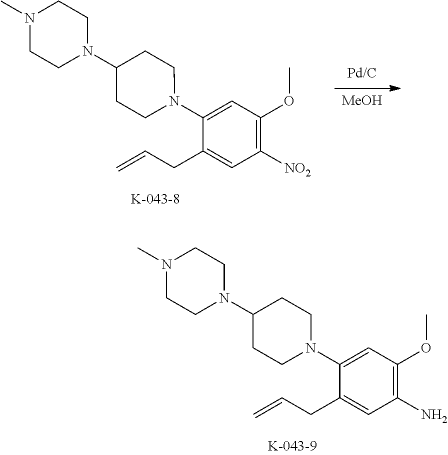 [SMILES: "MeOH" ; C=CCc1cc([N+](=O)[O-])c(OC)cc1N1CCC(N2CCN(C)CC2)CC1 ; "Pd/C" ; C=CCc1cc(N)c(OC)cc1N1CCC(N2CCN(C)CC2)CC1]

Dissolve K-043-8 (400 mg) in MeOH (20 mL), add Pd/C (200 mg) to the solution at room temperature, stir well, introduce H₂ and stir at room temperature for 3 hr. The reaction was complete as monitored by LCMS. After suction filtration, the filter cake was washed with methanol (20 mL). The organic phase was collected and concentrated to obtain the compound K-043-9 (200 mg), MS: 347.27 [M+H]⁺

### Step 5: Synthesis of Compound K-043

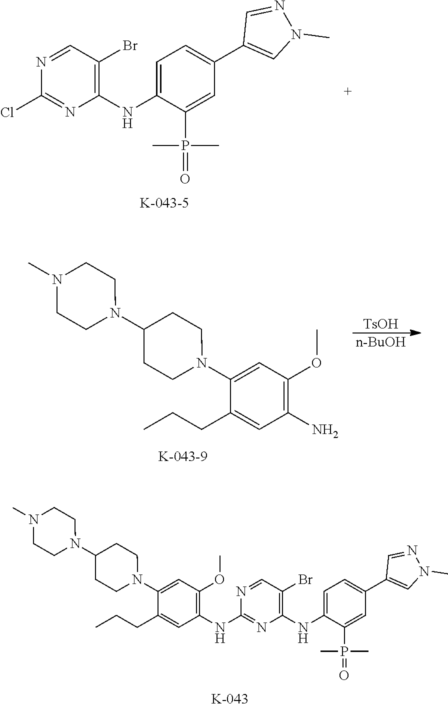

Add compound K-043-5 (100 mg), K-043-9 (78 mg), p-toluenesulfonic acid (23 mg) and n-butanol (2 mL) into the reaction flask, heat to 120°, and stir for 12 hr. The reaction was complete as monitored by LCMS, and then stopped. The reaction solution was poured into sodium carbonate solution (5 mL), and extracted with dichloromethane (3×50 mL); the organic phase was washed with saturated brine (3×30 mL), dried over anhydrous sodium sulfate, separated and purified by column chromatography (dichloromethane:methanol=12:1), and then concentrated to obtain off-white solid of compound K-043 (80 mg), MS: 750.29 [M+H]⁺

### Example 50: Synthesis of Compound K-045

### (2-((2-((5-Allyl-2-methoxy-4-(4-(4-methylpiperazin-1-yl)piperidin-1-yl)phenyl)amino)-5-bromopyrimidin-4-yl)amino)-5-(1-methyl-1H-pyrazol-4-yl)phenyl)dimethylphosphine oxide

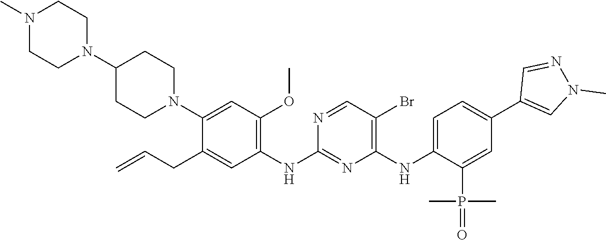

### Step 1: Synthesis of Compound K-045-7

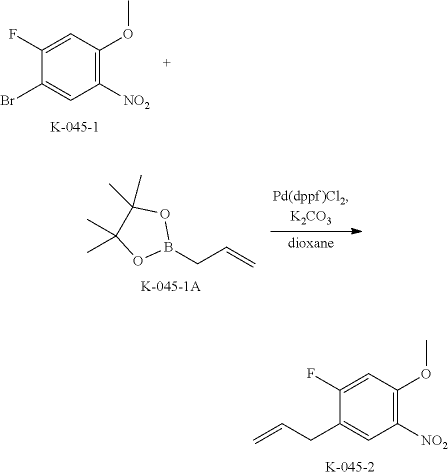

Add compounds K-045-1 (1.5 g), K-045-1A (2.02 g), Pd(dppf)Cl₂ (489 mg), K₂CO₃ (1.24 g), dioxane (30 mL) and water (3 mL) to the reaction flask, raise temperature to 100° C. under nitrogen protection and stir with heating for 1.5 hr. The reaction was complete as monitored by LCMS, and then stopped. The reaction solution was poured into water (50 mL), and extracted with dichloromethane (3×50 ml); the organic phase was washed with saturated brine (3×30 mL), dried over anhydrous sodium sulfate, separated and purified by column chromatography (N-hexane:ethyl acetate=1:1), concentrated and dried to obtain the yellow solid of desired product K-045-2 (1.0 g).

### Step 4: Synthesis of Compound K-045-3

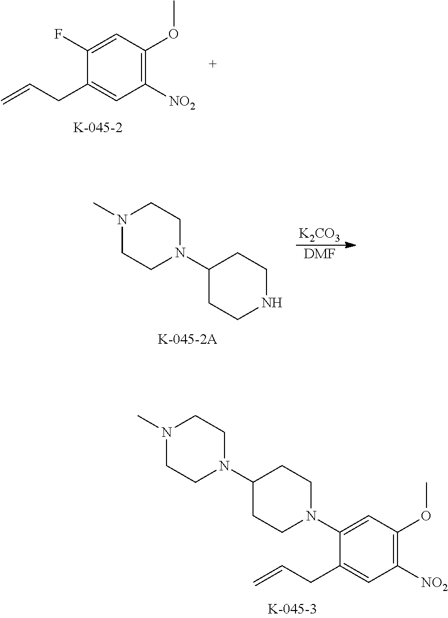

Dissolve K-045-2 (1.0 g) and K-045-2A (867 mg) in DMF (20 mL), and add K₂CO₃ (1.31 g) at room temperature; the gas in reactor was replaced with nitrogen for three times. After well stirring, the solution was transferred to an oil bath, heated to 100° C., and stirred at 100° C. for 16 hr. The reaction was complete as monitored by LCMS. The reaction solution was poured into water (50 mL), and extracted with dichloromethane (3×50 mL); the organic phase was washed with saturated brine (3×30 mL), dried over anhydrous sodium sulfate, separated and purified by column chromatography (N-hexane:ethyl acetate=1:1), then concentrated and dried to obtain desired product K-045-3 (400 mg), MS: 375.23 [M+H]⁺

### Step 5: Synthesis of Compound K-045-4

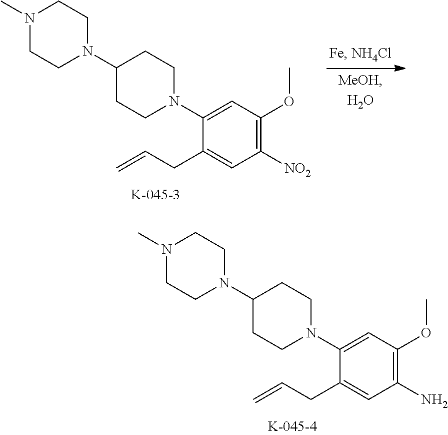 [SMILES: C=CCc1cc([N+](=O)[O-])c(OC)cc1N1CCC(N2CCN(C)CC2)CC1 ; C=CCc1cc(N)c(OC)cc1N1CCC(N2CCN(C)CC2)CC1]

Dissolve K-045-3 (400 mg) in MeOH (8 mL) and H₂O (8 mL), add iron powder (150 mg) and NH₄Cl (120 mg) to the solution; the gas in reactor was replaced with nitrogen for three times. After well stirring, the solution was transferred to an oil bath, heated to 80° C., and stirred at 80° C. for 3 hr. The reaction was complete as monitored by LCMS. After suction filtration, the filter cake was washed with methanol (20 mL). The reaction solution was poured into water (20 mL), and extracted with dichloromethane (3×50 mL); the organic phase was washed with saturated brine (3×30 mL), dried over anhydrous sodium sulfate, separated and purified by column chromatography (DCM:MeOH=10:1), then concentrated and dried to obtain desired product K-045-4 (180 mg), MS: 345.26 [M+H]⁺

### Step 6: Synthesis of Compound K-045

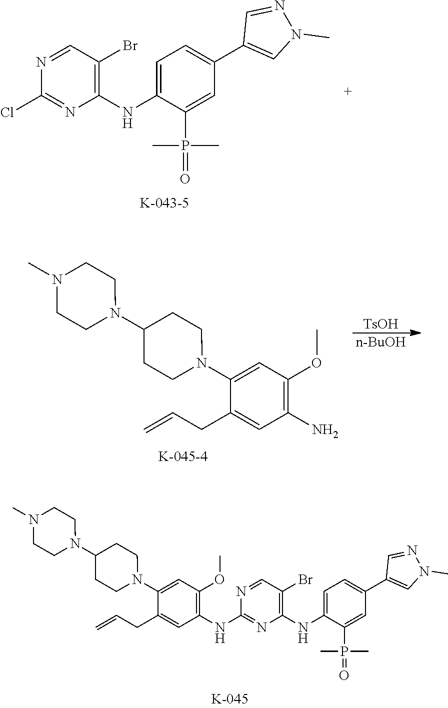

Add compound K-043-5 (100 mg), K-045-4 (78 mg), p-toluenesulfonic acid (23 mg) and n-butanol (2 mL) into the reaction flask, heat to 120° C., and stir for 12 hr. The reaction was complete as monitored by LCMS, and then stopped. The reaction solution was poured into sodium carbonate solution (5 mL), and extracted with dichloromethane (3×50 mL); the organic phase was washed with saturated brine (3×30 mL), dried over anhydrous sodium sulfate, separated and purified by column chromatography (dichloromethane:methanol=12:1), and then concentrated to obtain off-white solid of compound K-045 (45 mg), MS: 748.28 [M+H]⁺

### Example 51: Synthesis of Compound K-033

### (2-((5-Bromo-2-((5-methoxy-2-methyl-4-morpholinophenyl)amino)pyrimidin-4-yl)amino)-5-(5-cylopropylpyrazin-2-yl)phenyl)dimethylphosphine oxide

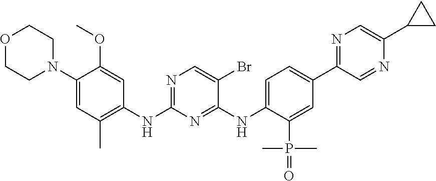 [SMILES: COc1cc(Nc2ncc(Br)c(Nc3ccc(-c4cnc(C5CC5)cn4)cc3P(C)(C)=O)n2)c(C)cc1N1CCOCC1]

### Step 1: Synthesis of Compound K-033-2

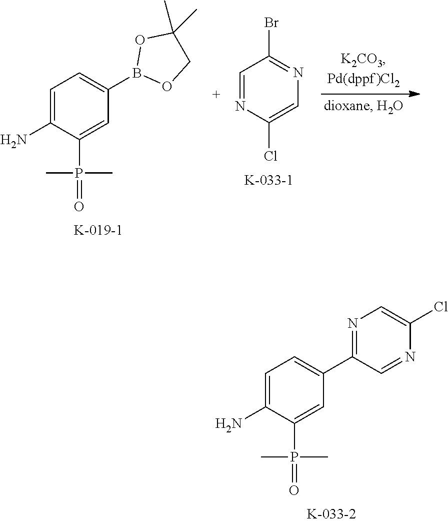 [SMILES: CC1(C)COB(c2ccc(N)c(P(C)(C)=O)c2)O1 ; Clc1cnc(Br)cn1 ; CP(C)(=O)c1cc(-c2cnc(Cl)cn2)ccc1N]

The compound K-033-2 was synthesized as the method described for compound K-022-6 using compound K-033-1 instead of raw compound K-022-5 to obtain pale brown powder of compound K-033-2, MS: 282.05 [M+H]⁺

### Step 2: Synthesis of Compound K-033-3

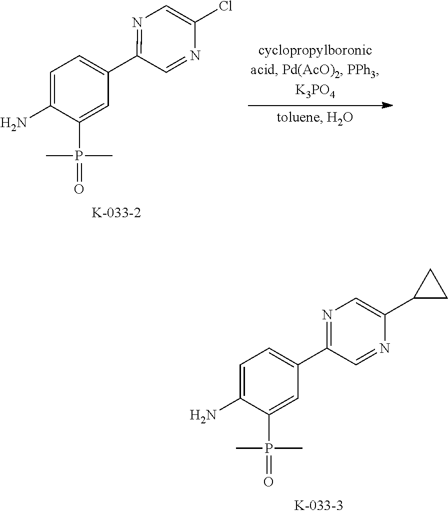 [SMILES: CP(C)(=O)c1cc(-c2cnc(Cl)cn2)ccc1N ; CP(C)(=O)c1cc(-c2cnc(C3CC3)cn2)ccc1N]

Add K-033-2 (160 mg), cyclopropylboronic acid (195 mg), palladium acetate (12.8 mg), triphenylphosphine (30 mg), potassium phosphate (362 mg), toluene (10 mL) and water (2 mL) to the reaction flask, heat to 100° C. under nitrogen protection. After complete reaction, the residue was spun off, and the residue was purified by column chromatography (dichloromethane:methanol=15:1) to obtain the yellow powder compound K-033-3 (82 mg), MS: 288.12 [M+H]⁺.

### Step 3: Synthesis of Compound K-033-4

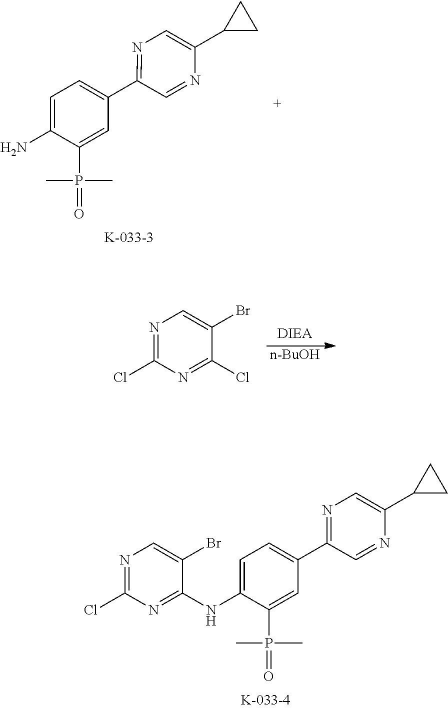

The compound K-033-4 was synthesized as the method described for compound K-004-5 using compound K-033-3 instead of raw compound K-004-4. For compound K-033-4, MS: 478.01 [M+H]⁺

### Step 4: Synthesis of Compound K-033

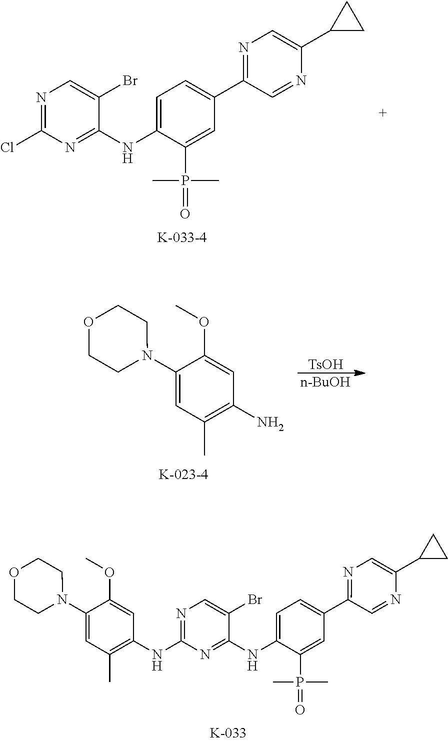

The compound K-033 was synthesized as the method described for compound K-018 using compound K-023-4 instead of raw compound K-018-9, compound K-033-4 instead of raw compound K-018-5 to obtain solid powder of hydrochloride salt of compound K-033 through the salt preparation process. For compound K-033, MS: 664.17 [M+H]⁺

### Example 52: Synthesis of Compound K-052

### (5-(Benzo[d]oxazol-4-yl)-2-((5-chloro-2-((5-ethyl-2-methoxy-4-(4-(4-methylpiperazin-1-yl)piperidin-1-yl)phenyl)amino)pyrimidin-4-yl)amino)phenyl)dimethylphosphine oxide

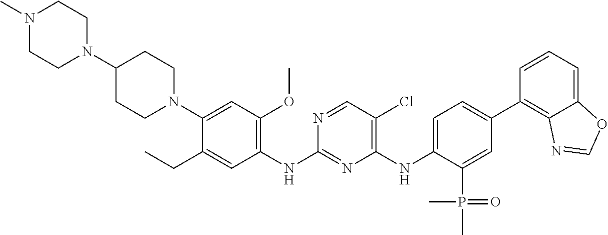 [SMILES: CCc1cc(Nc2ncc(Cl)c(Nc3ccc(-c4cccc5ocnc45)cc3P(C)(C)=O)n2)c(OC)cc1N1CCC(N2CCN(C)CC2)CC1]

### Step 1: Synthesis of Compound K-052-1

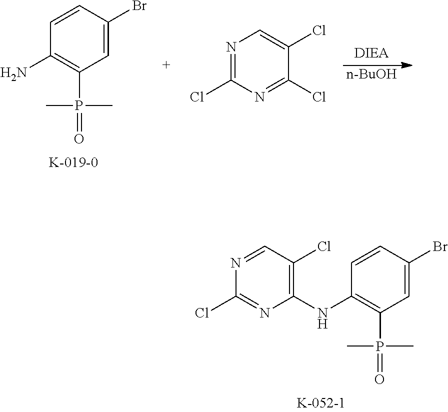

Add compound K-019-0 (1.0 g), 2,4,5-trichloropyrimidine (1.5 g), DIEA (1.3 g) and n-BuOH (20 ml) successively to the reaction flask, heat to 100° C., and stir with heating for 12 hr. The reaction was complete as monitored by LCMS, and then stopped. The reaction solution was poured into water (50 ml) solution, and extracted with ethyl acetate (3×50 mL); the organic phase was washed with saturated brine (3×30 ml), dried over anhydrous sodium sulfate, separated and purified by column chromatography (dichloromethane:methanol=20:1), and the solvent was removed to obtain yellow solid of desired product compound K-052-1 (700 mg), MS: 394 [M+H]⁺

### Step 2: Synthesis of Compound K-052-2

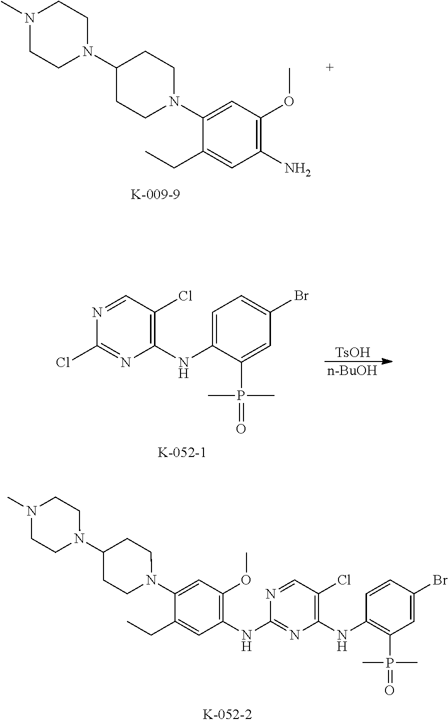

The compound K-052-2 was synthesized as the method described for compound K-018, that is compound K-009-9 reacts with compound K-052-1 to obtain compound K-052-2, MS: 690 [M+H]⁺

### Step 1: Synthesis of Compound K-052-3

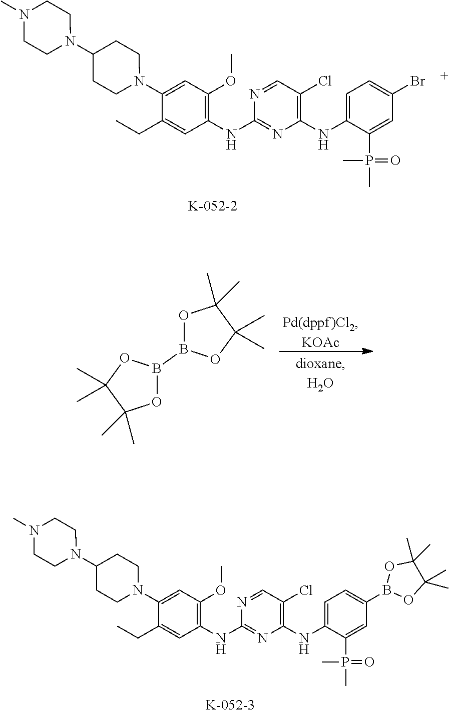

Add compound K-052-2 (1.5 g), bis(pinacolato)diborane (0.77 g), Pd(dppf)Cl₂ (0.18 g), potassium acetate (0.64 g) and dioxane (20 mL) successively to the reaction flask, heat to 100° C. under nitrogen protection and stir with heating for 3 hr. The reaction was complete as monitored by LCMS, and then stopped. The reaction solution was cooled down to room temperature, filtered and concentrated to obtain the crude product. The crude product was separated and purified by column chromatography (dichloromethane:methanol=15:1). The solvent was removed to get the yellow solid of desired product K-052-3 (0.8 g), MS: 738 [M+H]⁺

### Step 1: Synthesis of Compound K-052

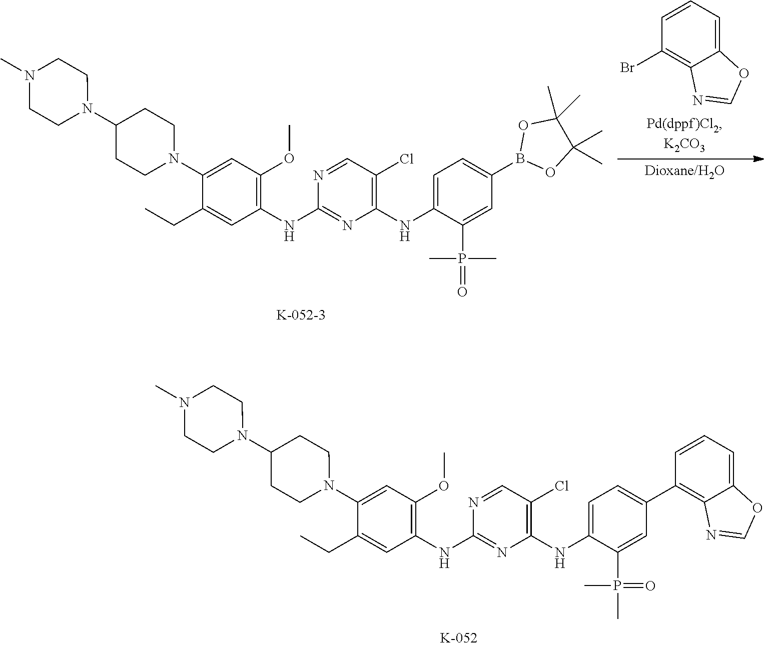

The compound K-052 was synthesized as the method described for compound K-060 using 4-Bromobenzo[d]oxazole instead of K-060-1. For compound K-052, MS: 729.31 [M+H]⁺

### Example 53: Synthesis of Compound K-053

### (5-(2-Aminothiazol-4-yl)-2-((5-chloro-2-((5-ethyl-2-methoxy-4-(4-(4-methylpiperazine-1-yl)piperidin-1-yl)phenyl)amino)pyrimidin-4-yl)amino)phenyl)dimethylphosphine oxide

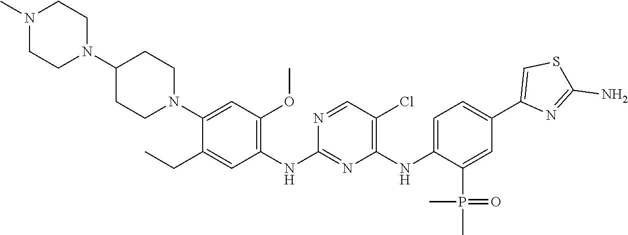 [SMILES: CCc1cc(Nc2ncc(Cl)c(Nc3ccc(-c4csc(N)n4)cc3P(C)(C)=O)n2)c(OC)cc1N1CCC(N2CCN(C)CC2)CC1]

### Step 1: Synthesis of Compound K-053

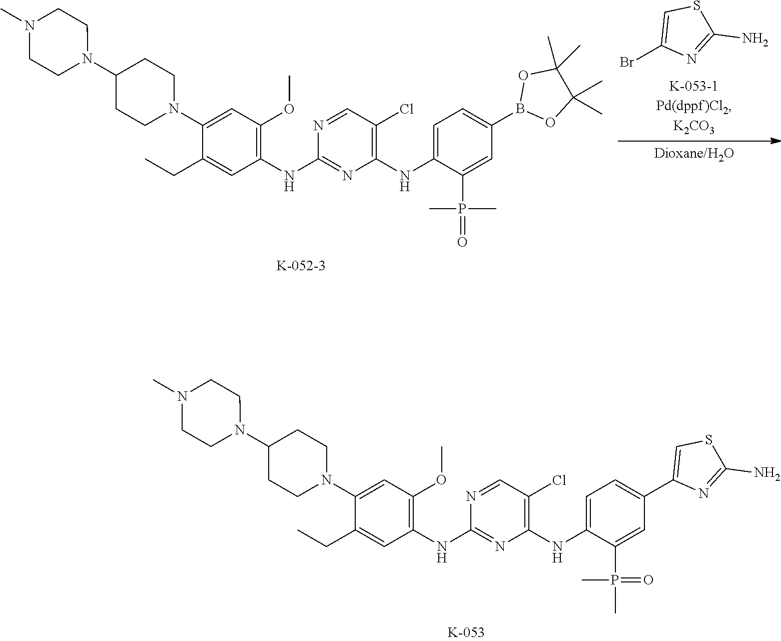

The compound K-053 was synthesized as the method described for compound K-060 using compound K-053-1 instead of raw compound K-060-2. For compound K-053, MS: 710.28 [M+H]⁺

### Example 54: Synthesis of Compound K-054

### (2-((5-Bromo-2-((5-ethyl-2-methoxy-4-(4-(4-methylpiperazin-1-yl)piperidin-1-yl)phenyl)amino)pyrimidin-4-yl)amino)-5-(4-methoxy-1H-pyrazol-1-yl)phenyl)dimethylphosphine oxide

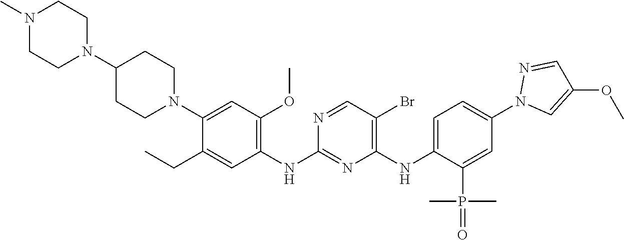

### Step 27: Synthesis of Compound K-054

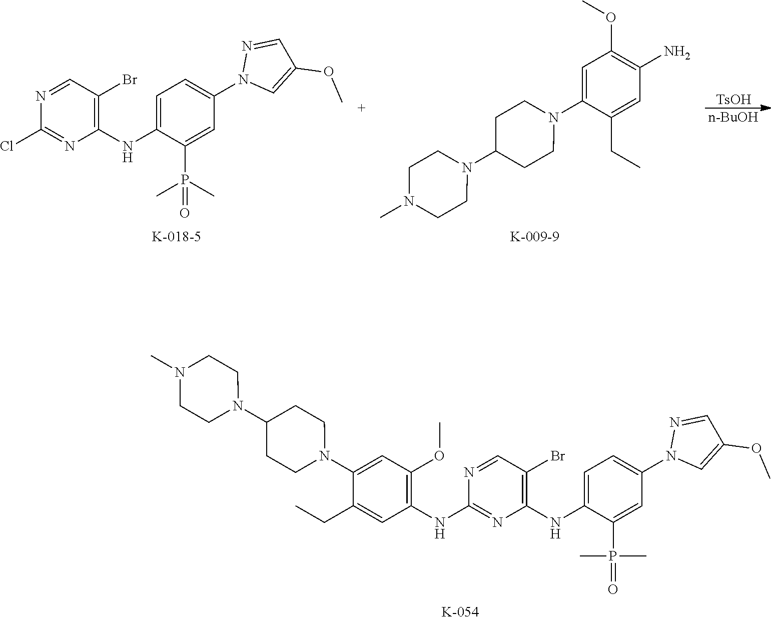

Add compound K-018-5 (100 mg), K-009-9 (76 mg), p-toluenesulfonic acid (60 mg) and n-butanol (2 mL) into the reaction flask, heat to 120° C., and stir for 12 hr. The reaction was complete as monitored by LCMS, and then stopped. The reaction solution was poured into sodium carbonate solution (5 mL), and extracted with dichloromethane (3×10 mL); the organic phase was washed with saturated brine (3×10 mL), and dried over anhydrous sodium sulfate; after positive phase separation (MeOH:H₂O, 0%-30%), the resulting product was extracted, and then separated and purified by column chromatography (dichloromethane:methanol=10:1), concentrated to obtain the off-white solid of compound K-054 (40 mg), MS: 752.27 [M+H]⁺

### Example 55: Synthesis of Compound K-055

### (5-(3-Aminopyridin-2-yl)-2-((5-chloro-2-((5-ethyl-2-methoxy-4-(4-(4-methylpiperazin-1-yl)piperidin-1-yl)phenyl)amino)pyrimidin-4-yl)amino)phenyl)dimethylphosphine oxide

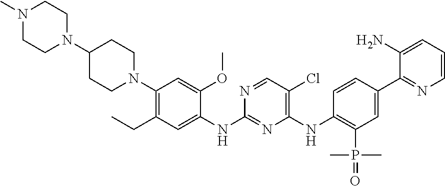 [SMILES: CCc1cc(Nc2ncc(Cl)c(Nc3ccc(-c4ncccc4N)cc3P(C)(C)=O)n2)c(OC)cc1N1CCC(N2CCN(C)CC2)CC1]

### Step 1: Synthesis of Compound K-055

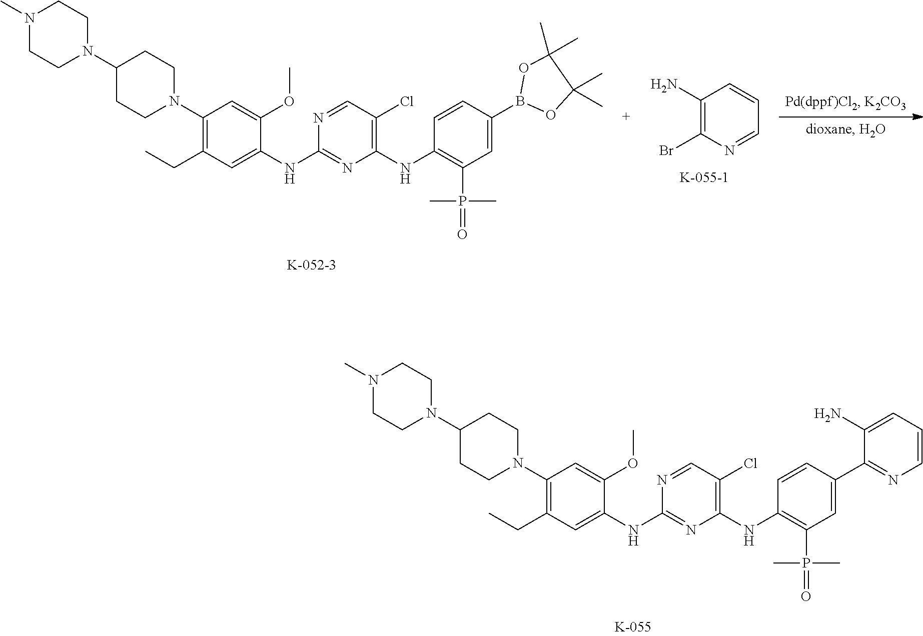

Add compounds K-052-3 (70 mg), K-055-1 (30 mg), Pd(dppf)Cl₂ (12 mg). K₂CO₃ (40 mg), dioxane (4 mL) and water (1 mL) successively to the reaction flask, raise temperature to 100° C. under nitrogen protection and stir with heating for 12 hr. The reaction was complete as monitored by LCMS, and then stopped. After removing the solvent, the residue was separated and purified by the preparative TLC (dichloromethane:methanol=6:1) to obtain the yellowish solid of desired compound K-055 (22.3 mg), MS: 704.33 [M+H]⁺

### Example 56: Synthesis of Compound K-056

### (2-((5-Bromo-2-((5-ethyl-2-methoxy-4-(4-(4-methylpiperazin-1-yl)piperidin-1-yl)phenyl)amino)pyrimidin-4-yl)amino)-5-(5-isopropylpyrimidin-2-yl)phenyl)dimethylphosphine oxide

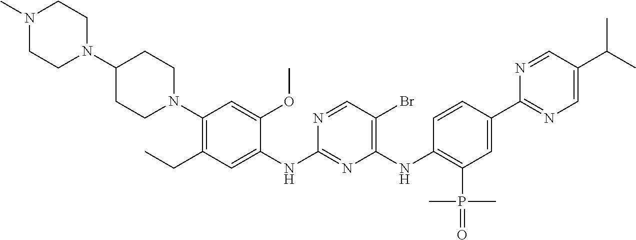 [SMILES: CCc1cc(Nc2ncc(Br)c(Nc3ccc(-c4ncc(C(C)C)cn4)cc3P(C)(C)=O)n2)c(OC)cc1N1CCC(N2CCN(C)CC2)CC1]

### Step 2: Synthesis of Compound K-056-1

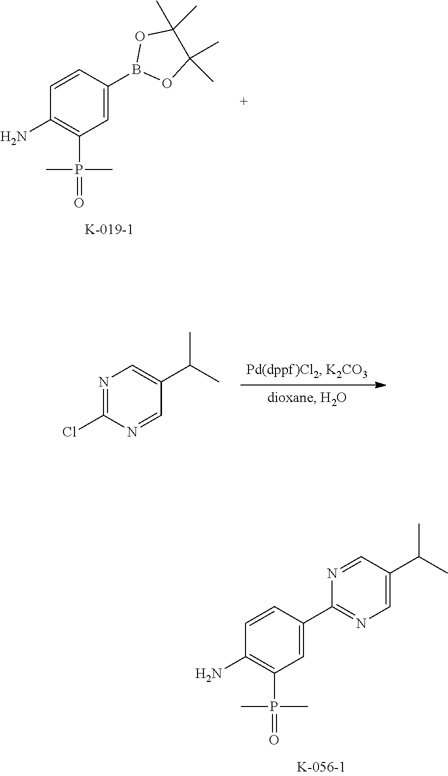

Add compounds K-019-1 (200 mg), 2-Chloro-5-isopropylpyrimidine (159 mg). Pd(dppf)Cl₂ (100 mg), K₂CO₃ (200 mg), dioxane (4 mL) and water (1 mL) successively to the reaction flask, raise temperature to 100° C. under nitrogen protection and stir with heating for 12 hr. The reaction was complete as monitored by LCMS, and then stopped. After removing the solvent, the residue was separated and purified by the preparative TLC (dichloromethane:methanol=20:1) to obtain the brown solid of desired product K-056-1 (40 mg), MS: 290 [M+H]⁺

### Step 3: Synthesis of Compound K-056-2

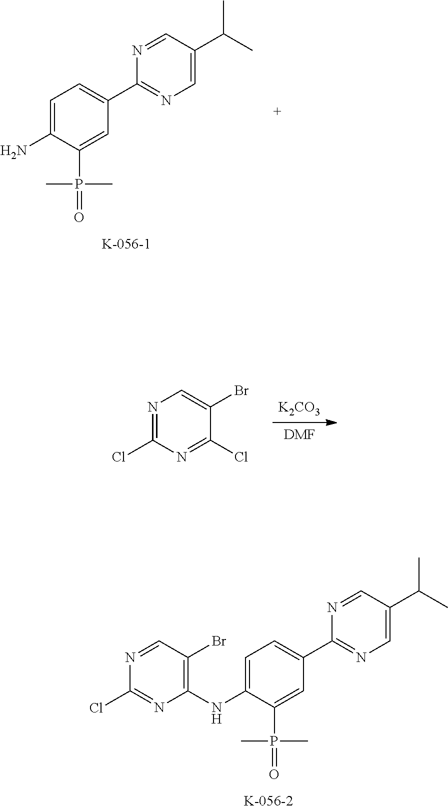

The compound K-056-2 was synthesized as the method described for compound K-018-5 using compound K-056-1 instead of raw compound K-018-4. For compound K-056-2, MS: 480 [M+H]⁺

### Step 4: Synthesis of Compound K-056

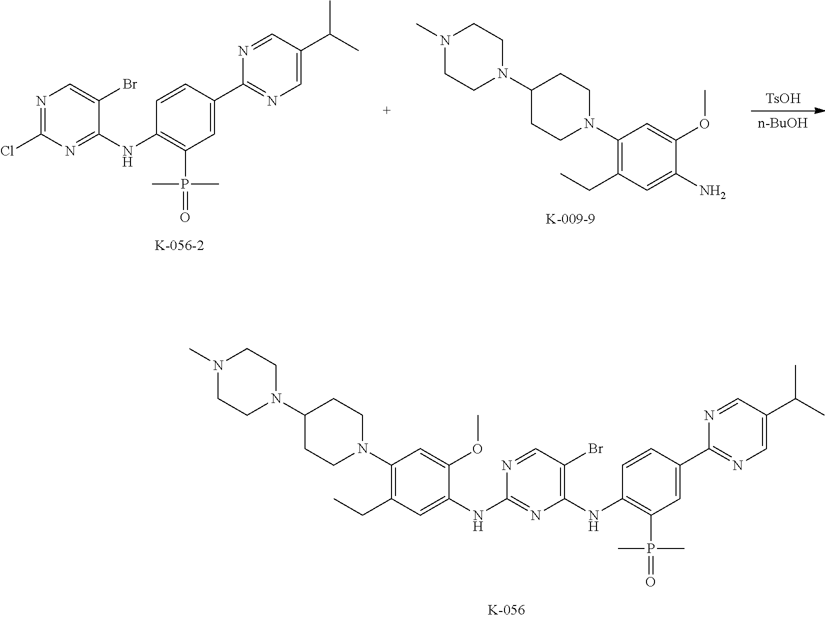

The compound K-056 was synthesized as the method described for compound K-018 using compound K-009-9 instead of raw compound K-056-2. For compound K-056, MS: 776.31 [M+H]⁺

### Example 57: Synthesis of Compound K-057

### (2-((5-Bromo-2-((5-ethyl-2-methoxy-4-(4-(4-methylpiperazin-1-yl)piperidin-1-yl)phenyl)amino)pyrimidin-4-yl)amino)-5-(2-(dimethylamino)pyrimidin-5-yl)phenyl)dimethylphosphine oxide

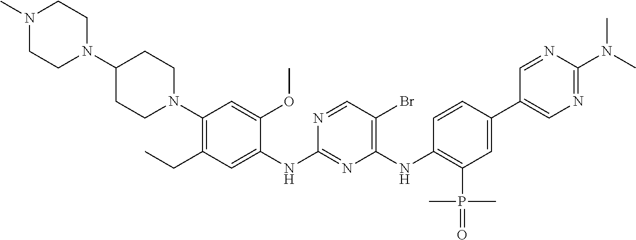

### Step 1: Synthesis of Compound K-057-0

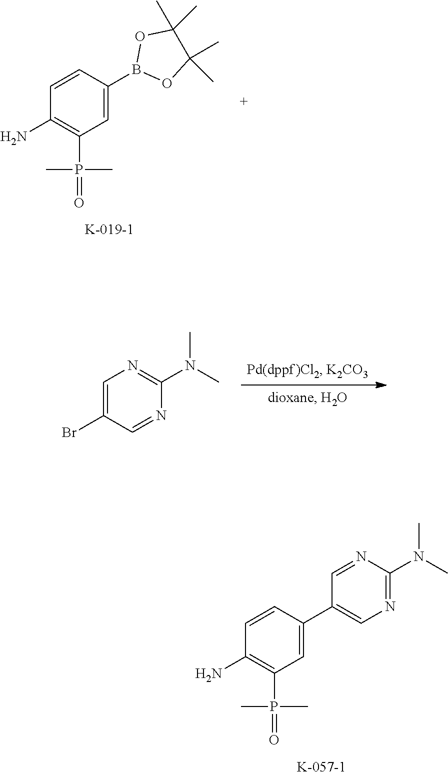

The compound K-057-1 was synthesized as the method described for compound K-019-2, that is compound K-019-1 reacted with compound 5-Bromo-N,N-dimethylpyrimidin-2-amine to produce compound K-057-1, MS: 291 [M+H]⁺

### Step 2: Synthesis of Compound K-057-2

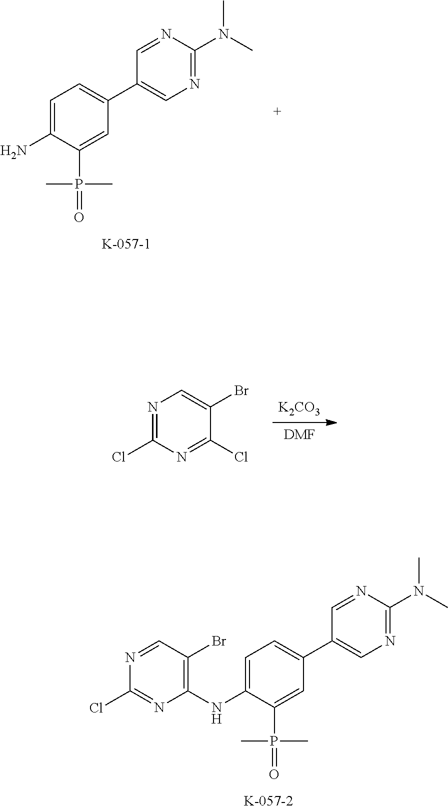

The compound K-057-2 was synthesized as the method described for compound K-018-5, that is compound K-057-1 reacted with compound 5-Bromo-2,4-dichloropyrimidine to produce compound K-057-2, MS: 481 [M+H]⁺

### Step 3: Synthesis of Compound K-057

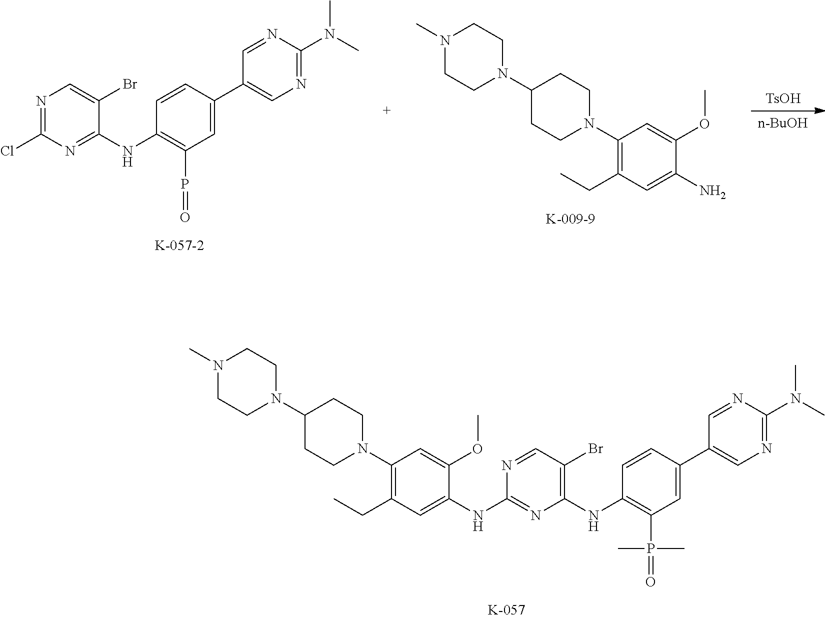

The compound K-057 was synthesized as the method described for compound K-018, that is compound K-009-9 reacted with compound K-057-2 to produce compound K-057, MS: 777.30 [M+H]⁺

### Example 58: Synthesis of Compound K-058

### (2-((5-Bromo-2-((5-ethyl-2-methoxy-4-(4-(4-methylpiperazin-1-yl)piperidin-1-yl)phenyl)amino)pyrimidin-4-yl)amino)-5-(5-ethoxypyrimidin-2-yl)phenyl)dimethylphosphine oxide

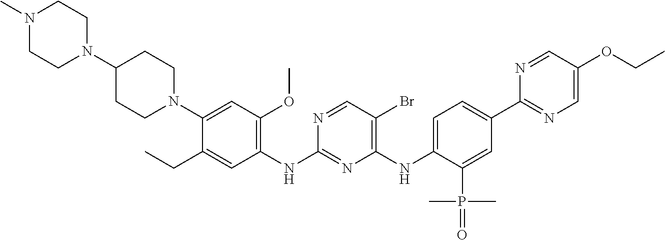 [SMILES: CCOc1cnc(-c2ccc(Nc3nc(Nc4cc(CC)c(N5CCC(N6CCN(C)CC6)CC5)cc4OC)ncc3Br)c(P(C)(C)=O)c2)nc1]

### Step 1: Synthesis of Compound K-058-1

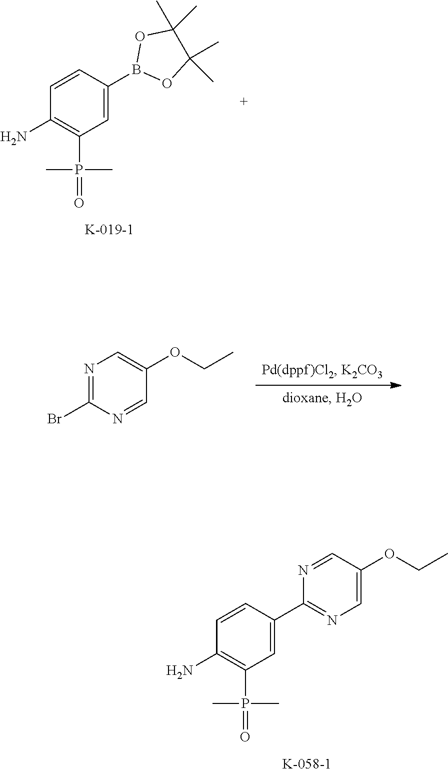

The compound K-058-1 was synthesized as the method described for compound K-019-2, that is compound K-019-1 reacted with 2-Bromo-5-ethoxypyrimidine to produce compound K-058-1, MS: 292.111 [M+H]⁺

### Step 2: Synthesis of Compound K-057-2

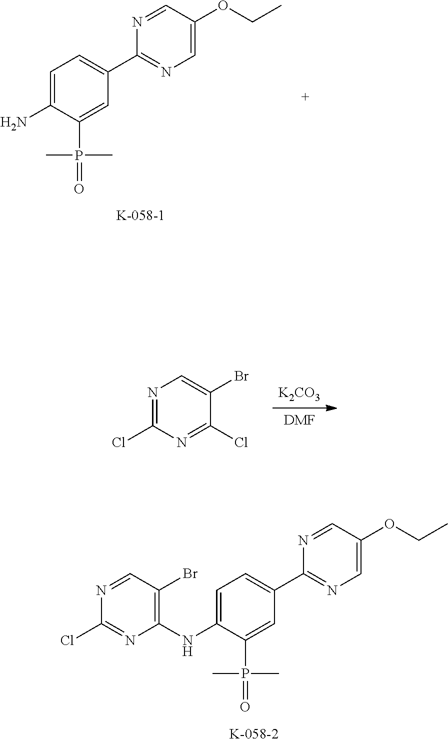

The compound K-058-2 was synthesized as the method described for compound K-018-5, that is compound K-058-1 reacted with 5-Bromo-2,4-dichloropyrimidine to produce compound K-058-2, MS: 482.01 [M+H]⁺

### Step 3: Synthesis of Compound K-058

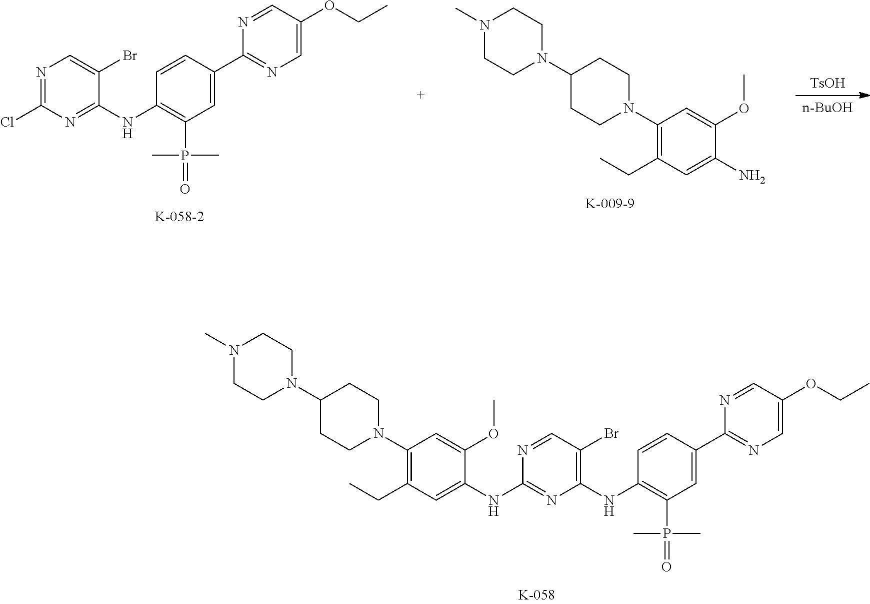

The compound K-058 was synthesized as the method described for compound K-018, that is compound K-058-2 reacted with compound K-009-9 to produce compound K-058, MS: 778.29 [M+H]⁺

### Example 59: Synthesis of Compound K-059

### (2-((5-Bromo-2-((5-ethyl-2-methoxy-4-(4-(4-methylpiperazin-1-yl)piperidin-1-yl)phenyl)amino)pyrimidin-4-yl)amino)-5-(2-ethoxypyrimidin-5-yl)phenyl)dimethylphosphine oxide

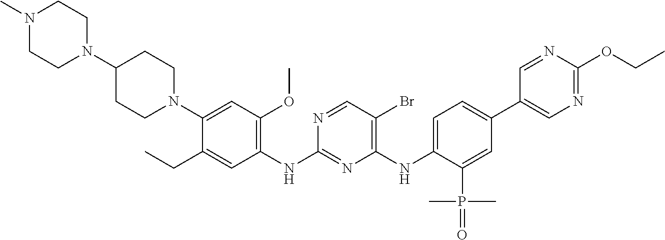 [SMILES: CCOc1ncc(-c2ccc(Nc3nc(Nc4cc(CC)c(N5CCC(N6CCN(C)CC6)CC5)cc4OC)ncc3Br)c(P(C)(C)=O)c2)cn1]

### Step 1: Synthesis of Compound K-059-1

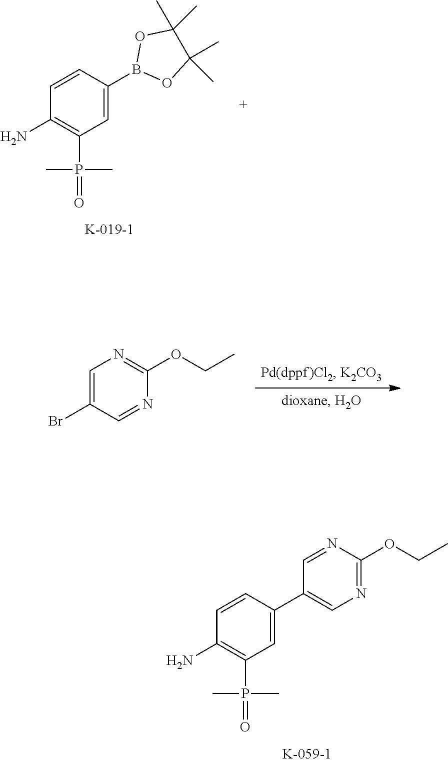

The compound K-059-1 was synthesized as the method described for compound K-019-2 using compound 5-Bromo-2-ethoxypyrimidine instead of compound K-019-1. For compound K-059-1, MS: 292.11 [M+H]⁺

### Step 2: Synthesis of Compound K-059-2

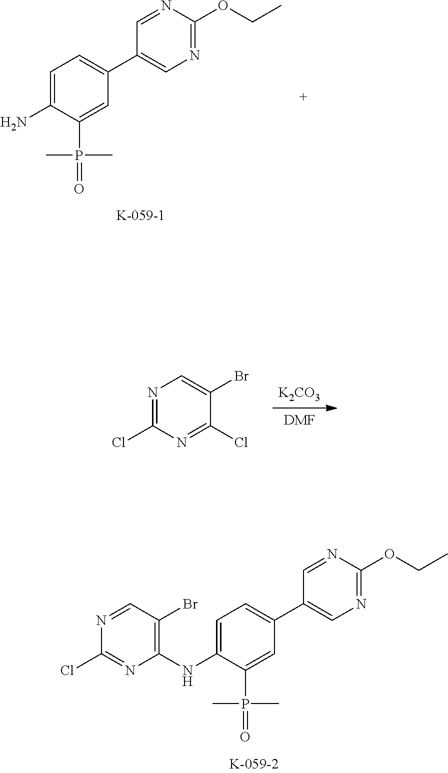

The compound K-059-2 was synthesized as the method described for compound K-018-5, that is 5-Bromo-2,4-dichloropyrimidine reacted with compound K-059-1 to produce compound K-057-2, MS: 482.01 [M+H]⁺

### Step 3: Synthesis of Compound K-059

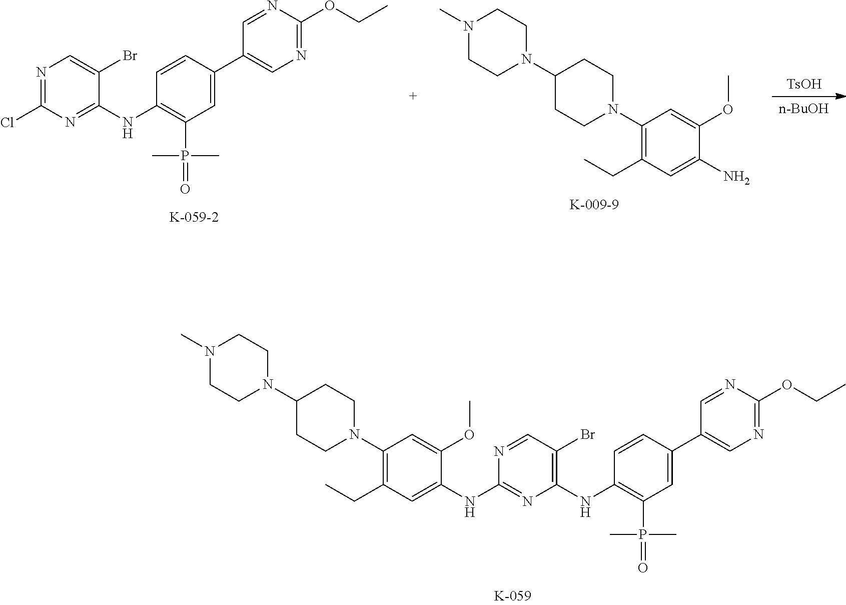

The compound K-059 was synthesized as the method described for compound K-018, that is compound K-059-2 reacted with compound K-009-9 to produce compound K-059, MS: 778.29 [M+H]⁺

### Example 60: Synthesis of Compound K-060

### 3-(4-((5-Chloro-2-((5-ethyl-2-methoxy-4-(4-(4-methylpiperazin-1-yl)piperidin-1-yl)phenyl)amino)pyrimidin-4-yl)amino)-3-(dimethylphosphine oxide)phenyl)-1-methyl-1H-pyrazol-4-carbonitrile

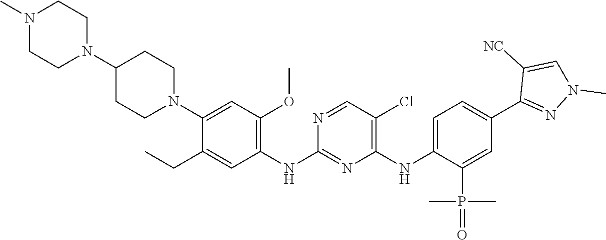

### Step 1: Synthesis of Compound K-060

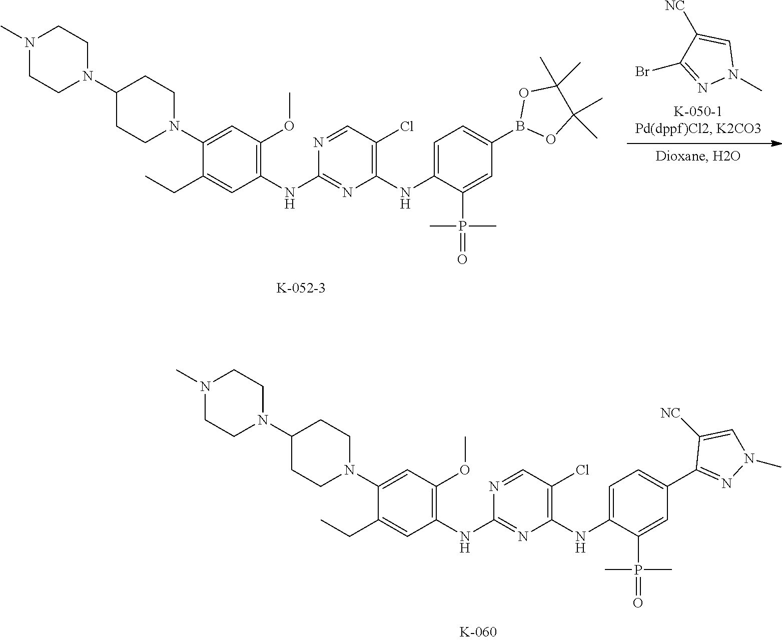

Add compounds K-052-3 (100 mg), K-060-1 (76 mg), Pd(dppf)Cl₂ (22 mg), and K₂CO₃ (86 mg) successively to dioxane (8 mL) and water (2 ml), heat to 100° C. and stir for 12 hr. The reaction was complete as monitored by LCMS, and then stopped. The reaction solution was cooled down to room temperature, poured into water (5 mL), and extracted with dichloromethane (3×10 mL); the organic phase was washed with saturated brine (3×10 mL), dried over anhydrous sodium sulfate, separated and purified by column chromatography (dichloromethane:methanol=10:1), then concentrated to obtain the off-white solid product K-060 (80 mg), MS: 717.32 [M+H]⁺

### Example 61: Synthesis of Compound K-061

### (2-((5-Chloro-2-((5-ethyl-2-methoxy-4-(4-(4-methylpiperazin-1-yl)piperidin-1-yl)phenyl)amino)pyrimidin-4-yl)amino)-5-(3-ethoxy-5-methylisoxazol-4-yl)phenyl)dimethylphosphine oxide

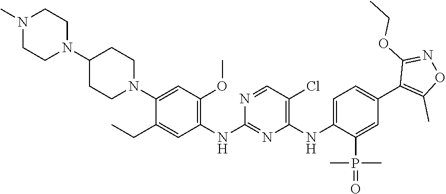 [SMILES: CCOc1noc(C)c1-c1ccc(Nc2nc(Nc3cc(CC)c(N4CCC(N5CCN(C)CC5)CC4)cc3OC)ncc2Cl)c(P(C)(C)=O)c1]

### Step 1: Synthesis of Compound K-061

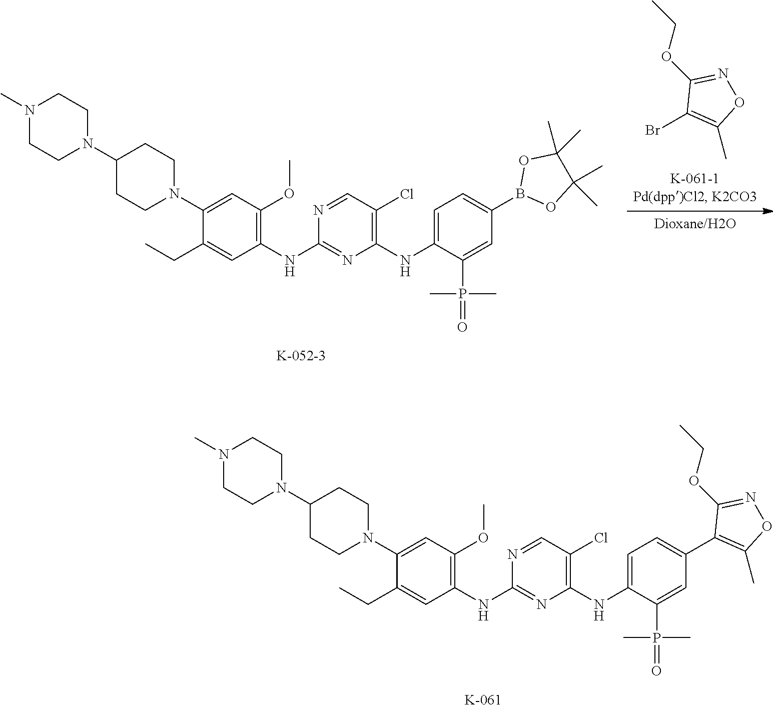

Add compounds K-052-3 (100 mg), K-060-1 (78 mg), Pd(dppf)Cl₂ (22 mg), and K₂CO₃ (86 mg) successively to dioxane (8 mL) and water (2 ml), heat to 100° C. and stir for 12 hr. The reaction was complete as monitored by LCMS, and then stopped. The reaction solution was cooled down to room temperature, poured into water (5 mL), and extracted with dichloromethane (3×10 mL); the organic phase was washed with saturated brine (3×10 mL), dried over anhydrous sodium sulfate, separated and purified by column chromatography (dichloromethane:methanol=10:1), then concentrated to obtain the oft-white solid compound K-061 (10 mg), MS: 737.34 [M+H]⁺

### Example 62: Synthesis of Compound K-062

### (2-((5-Bromo-2-((5-ethyl-2-methoxy-4-(4-(4-methylpiperazin-1-yl)piperidin-1-yl)phenyl)amino)pyrimidin-4-yl)amino)-5-(2-ethyl-2H-1,2,3-triazol-4-yl)phenyl)dimethylphosphine oxide

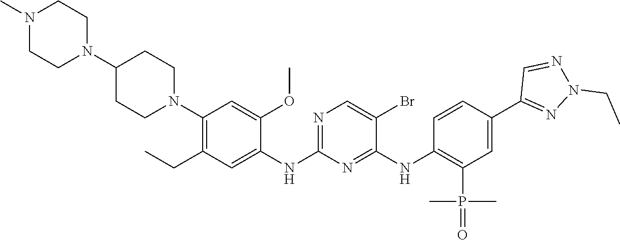 [SMILES: CCc1cc(Nc2ncc(Br)c(Nc3ccc(-c4cnn(CC)n4)cc3P(C)(C)=O)n2)c(OC)cc1N1CCC(N2CCN(C)CC2)CC1]

Step 1: Synthesis of Compound K-062-1

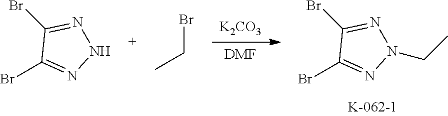

The compound K-062-1 was synthesized as the method described for compound K-068-1 using compound bromoethane instead of compound 2-Bromopropane. For compound K-062-3, MS: 253.89 [M+H]⁺

### Step 2: Synthesis of Compound K-062-2

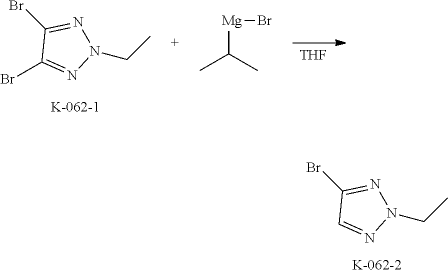

The compound K-062-2 was synthesized as the method described for compound K-068-2 using compound K-062-1 instead of raw compound K-068-1. For compound K-062-2, MS: 175.97 [M+H]⁺

### Step 3: Synthesis of Compound K-062-3

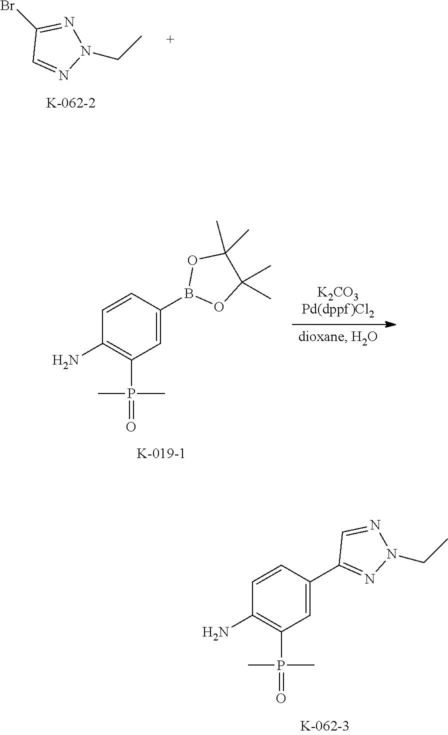

The compound K-062-3 was synthesized as the method described for compound K-019-2 using compound K-062-2 instead of raw compound 2-Bromothiazole. For compound K-062-3, MS: 265.11 [M+H]⁺

### Step 4: Synthesis of Compound K-062-4

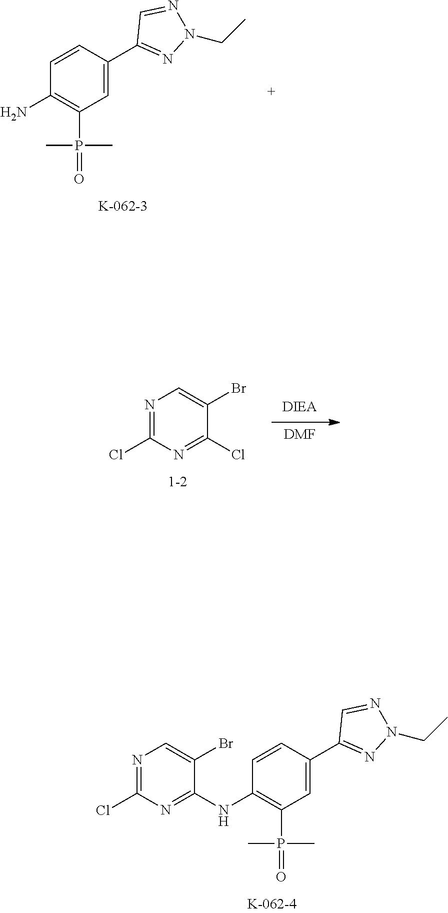

The compound K-062-4 was synthesized as the method described for compound K-018-5 using compound K-062-3 instead of raw compound K-018-4. For compound K-062-4, MS: 455.01 [M+H]⁺

### Step 5: Synthesis of Compound K-062

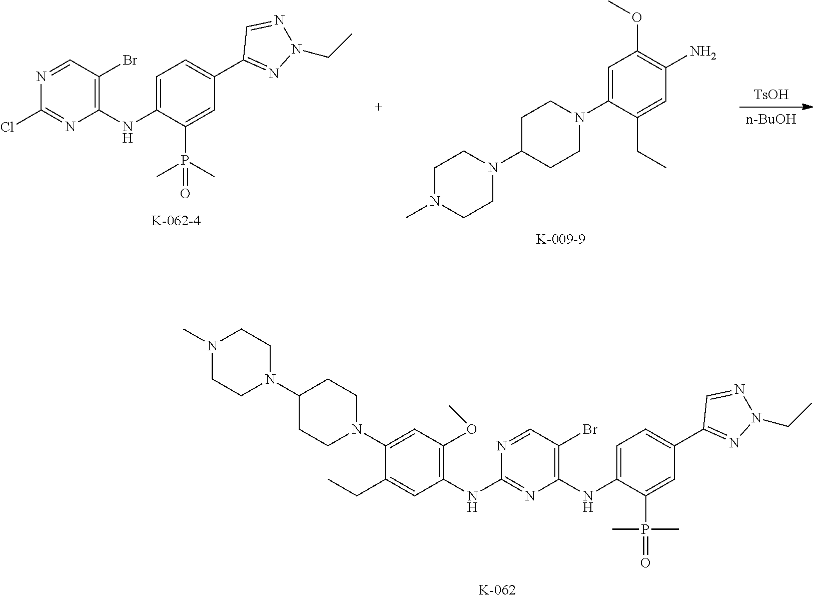

The compound K-062 was synthesized as the method described for compound K-018 using compound K-009-9 instead of raw compound K-018-9, compound K-062-4 instead of raw compound K-018-5. For compound K-062, MS: 751.29 [M+H]⁺

### Example 63: Synthesis of Compound K-063

### (2-((5-Bromo-2-((5-ethyl-2-methoxy-4-(4-(4-methylpiperazin-1-yl)piperidin-1-yl)phenyl)amino)pyrimidin-4-yl)amino)-5-(pyrazin-2-yl)phenyl)dimethylphosphine oxide

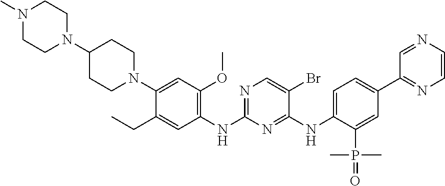 [SMILES: CCc1cc(Nc2ncc(Br)c(Nc3ccc(-c4cnccn4)cc3P(C)(C)=O)n2)c(OC)cc1N1CCC(N2CCN(C)CC2)CC1]

### Step 1: Synthesis of Compound K-057-1

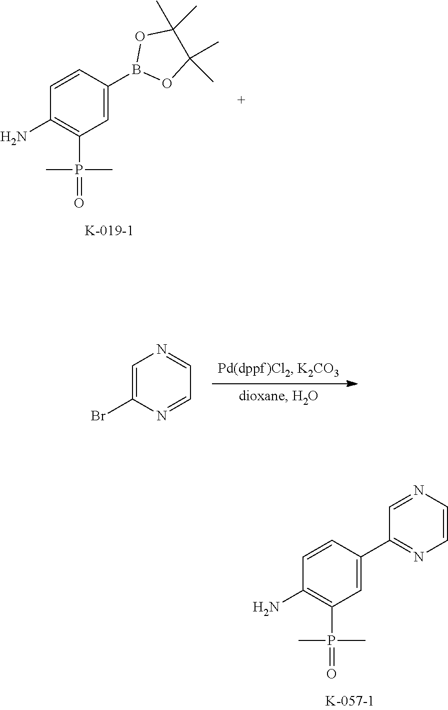 [SMILES: CC1(C)OB(c2ccc(N)c(P(C)(C)=O)c2)OC1(C)C ; Brc1cnccn1 ; "Pd(dppf)Cl2, K2CO3" ; "dioxane, H2O" ; CP(C)(=O)c1cc(-c2cnccn2)ccc1N]

The compound K-057-1 was synthesized as the method described for compound K-019-2, that is compound K-019-1 reacted with compound 2-Bromopyrazine to produce compound K-057-1, MS: 248.09 [M+H]⁺

### Step 2: Synthesis of Compound K-057-2

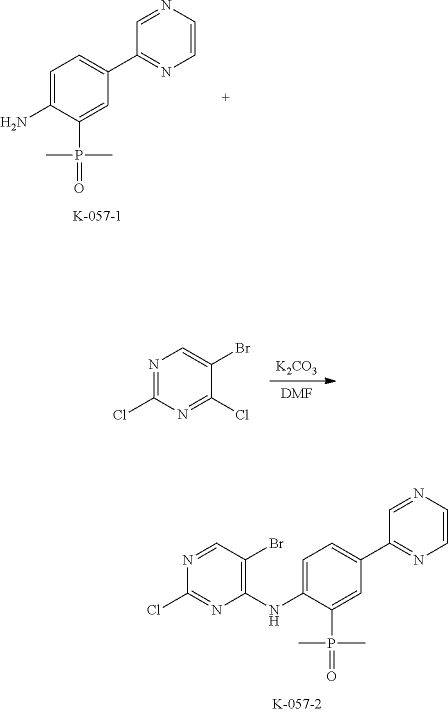

The compound K-057-2 was synthesized as the method described for compound K-018-5, that is 5-Bromo-2,4-dichloropyrimidine reacted with compound K-057-1 to produce compound K-057-2, MS: 437.98 [M+H]⁺

### Step 3: Synthesis of Compound K-057

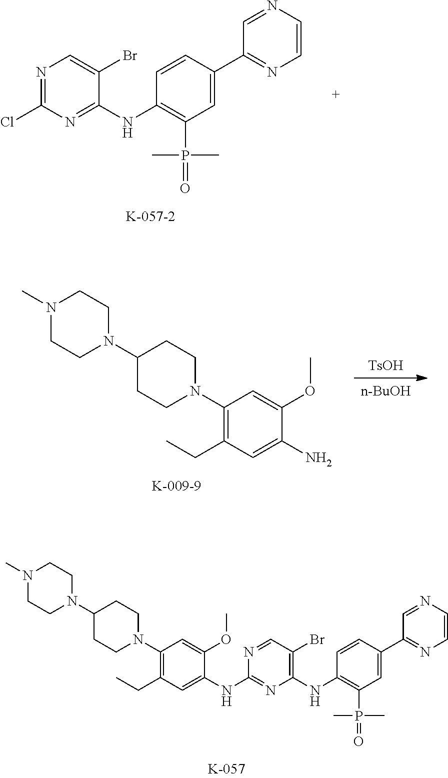

The compound K-057 was synthesized as the method described for compound K-018, that is compound K-057-2 reacted with compound K-009-9 to produce compound K-057, MS: 734.26 [M+H]⁺

### Example 64: Synthesis of Compound K-064

(2-((5-Bromo-2-((5-ethyl-2-methoxy-4-(4-(4-methylpiperazin-1-yl)piperidin-1-yl)phenyl)amino)pyrimidin-4-yl)amino)-5-(5-cyclopropylpyrazin-2-yl)phenyl)dimethylphosphine oxide

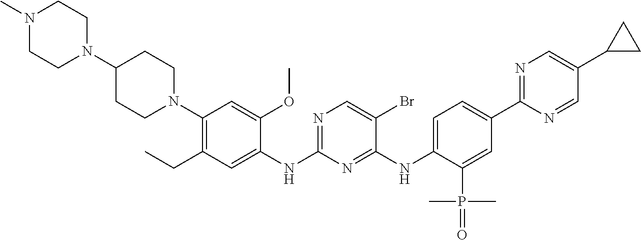 [SMILES: CCc1cc(Nc2ncc(Br)c(Nc3ccc(-c4ncc(C5CC5)cn4)cc3P(C)(C)=O)n2)c(OC)cc1N1CCC(N2CCN(C)CC2)CC1]

### Step 1: Synthesis of Compound K-064-1

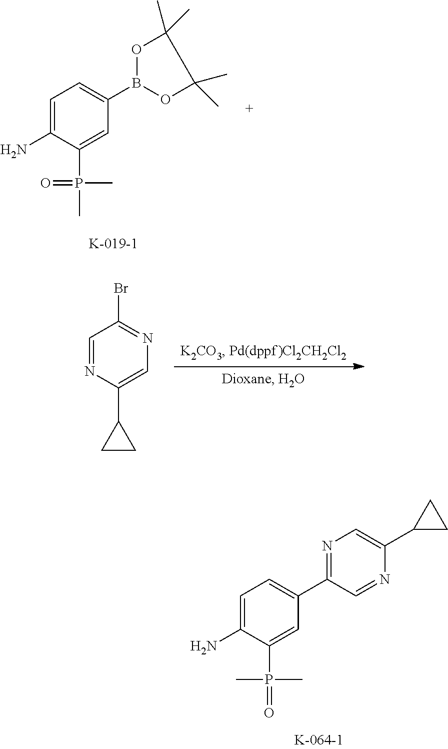 [SMILES: CC1(C)OB(c2ccc(N)c(P(C)(C)=O)c2)OC1(C)C ; Brc1cnc(C2CC2)cn1 ; "Dioxane, H2O" ; "K2CO3, Pd(dppf)Cl2CH2Cl2" ; CP(C)(=O)c1cc(-c2cnc(C3CC3)cn2)ccc1N]

Add compounds K-064-1 (300 mg), 2-Brumo-5-cyclopenlyrazine (202 mg), K2CO3 (281 mg). Pd(dppf)Cl2CH2Cl2 (83 mg), dioxane (5 mL) and water (2 mL) successively to the reaction flask, raise temperature to 100° C. under nitrogen protection and stir with heating for 12 hr. The reaction was complete as monitored by LCMS, and then stopped. The solution was separated and purified by column chromatography (dichloromethane:methanol=15:1), and then concentrate to obtain the brown solid of desired product K-064-1 (278 mg), MS: 288.12[M+H]⁺

### Step 2: Synthesis of Compound K-064-2

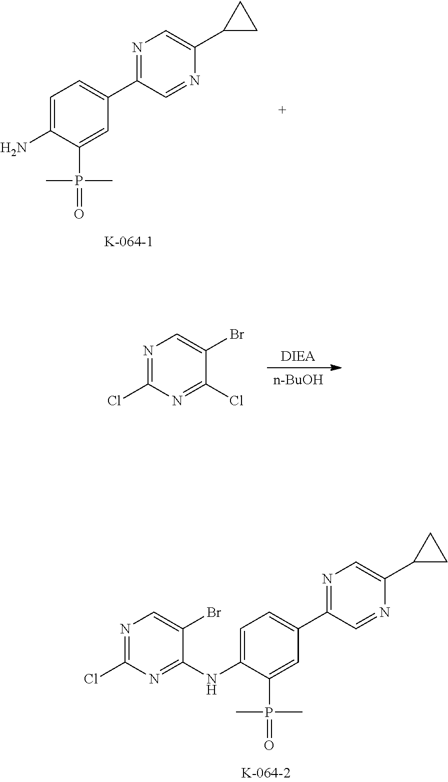

Add compound K-064-1 (278 mg), 5-Bromo-2,4-dichloropyrimidine (1.1 g), DIEA (375 mg) and n-BuOH (8 mL) successively to the reaction flask, heat to 110° C. under nitrogen protection, and stir with heating for 12 hr. The reaction was complete as monitored by LCMS, and then stopped. The solid is precipitated during reaction, filtered, washed with n-butanol, diethyl ether successively, and dried to obtain the brown solid of desired product compound K-064-2 (328 mg), MS: 478.01 [M+H]⁺

### Step 3: Synthesis of Compound K-064

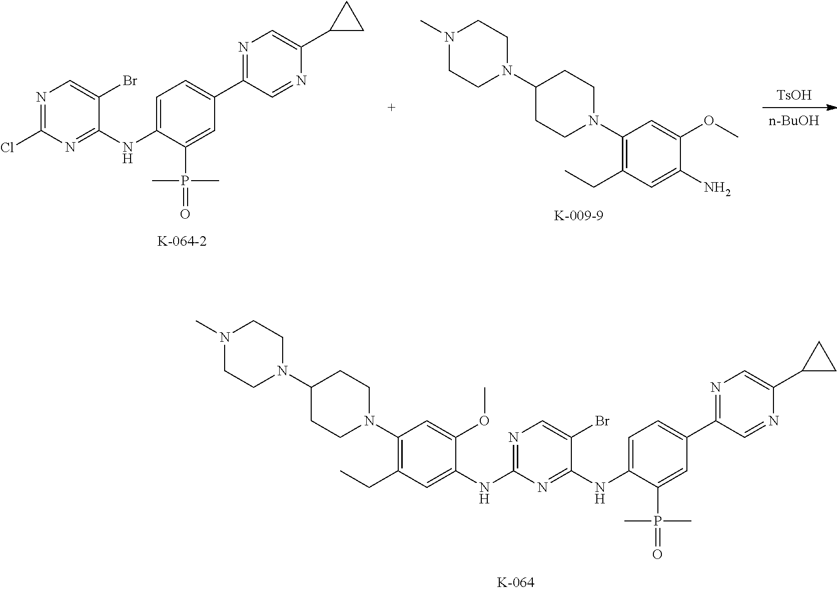

Add compounds K-064-2 (328 mg), K-009-9 (228 mg), p-toluenesulfonic acid (236 mg) and n-butanol (5 mL) successively to the reaction flask, heat to 100° C. under nitrogen protection and stir for 12 hr. The reaction was complete as monitored by LCMS, and then stopped. Triethylamine was added dropwise to the solution with pH value of 8, and then concentrated; the residue was separated and purified with a thick preparative plate (dichloromethane:methanol=7:1), and the eluted product was concentrated to obtain the off-white solid of compound K-064 (111 mg), MS: 774.29 [M+H]⁺

### Example 65: Synthesis of Compound K-065

### (2-((5-Bromo-2-((5-ethyl-2-methoxy-4-(4-(4-methylpiperazin-1-yl)piperidin-1-yl)phenyl)amino)pyrimidin-4-yl)amino)-5-(5-methylpyrazin-2-yl)phenyl)dimethylphosphine oxide

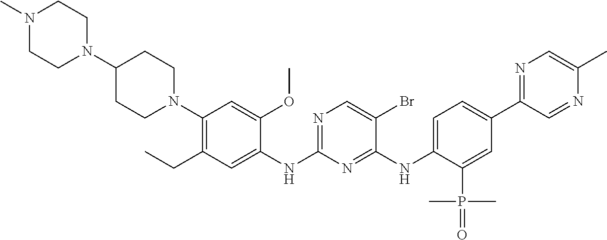

### Step 1: Synthesis of Compound K-065-1

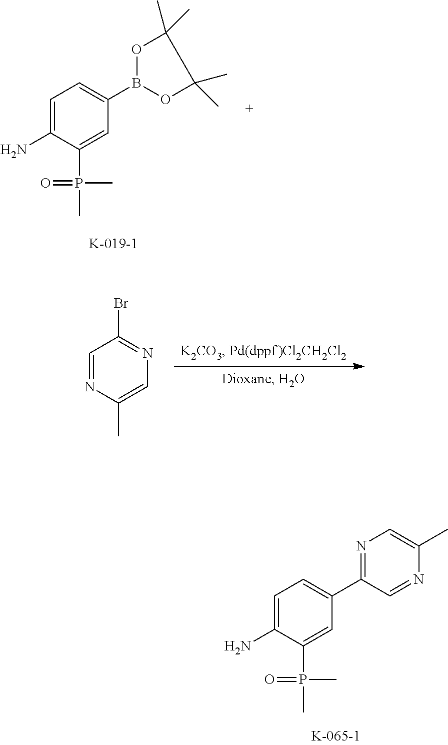

The compound K-065-1 was synthesized as the method described for compound K-019-2, that is compound K-019-1 reacted with 2-Bromo-5-methylpyrazine (200 mg) to produce a solid powder of compound K-065-1 (134 mg), MS: 262.10 [M+H]⁺.

### Step 2: Synthesis of Compound K-065-2

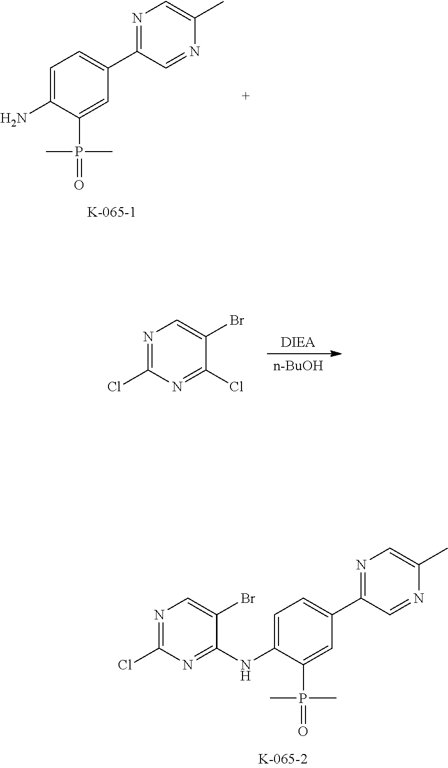

The compound K-065-2 was synthesized as the method described for compound K-018-5, that is 5-Bromo-2,4-dichloropyrimidine reacted with compound K-065-1 (113 mg) to produce a solid powder of compound K-065-2 (220 mg), MS: 452.00 [M+H]⁺.

### Step 2: Synthesis of Compound K-065

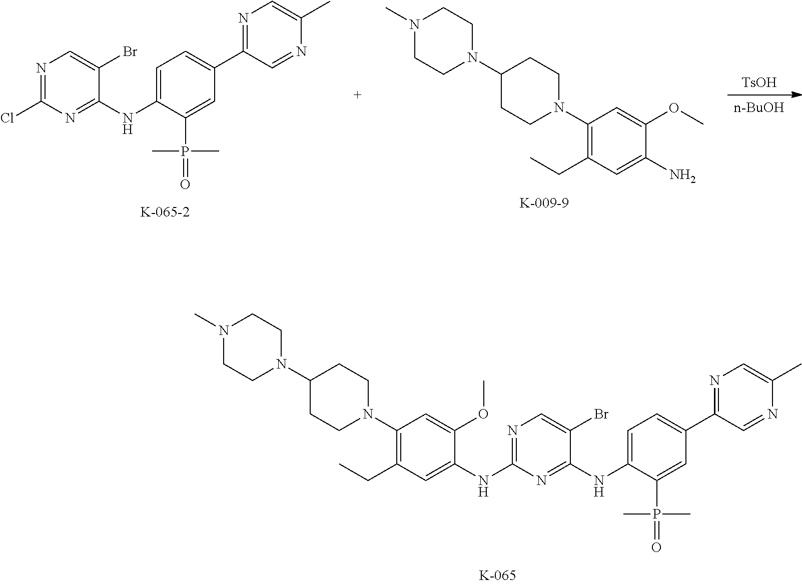

The compound K-065 was synthesized as the method described for compound K-018, that is compound K-009-9 reacted with compound K-065-2 (220 mg) to produce a solid powder of compound K-065 (65 mg), MS: 748.28 [M+H]⁺.

### Example 66: Synthesis of Compound K-066

### (2-((5-Bromo-2-((5-ethyl-2-methoxy-4-(4-(4-methylpiperazin-1-yl)piperidin-1-yl)phenyl)amino)pyrimidin-4-yl)amino)-5-(1,5-dimethyl-1H-pyrol-3-yl)phenyl)dimethylphosphine oxide

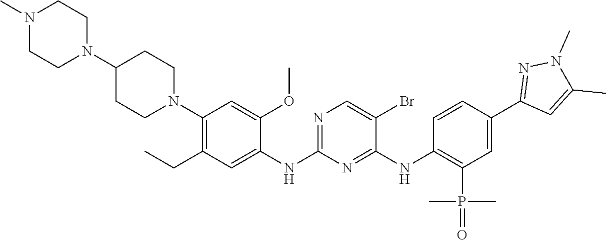

### Step 2: Synthesis of Compound K-066-1

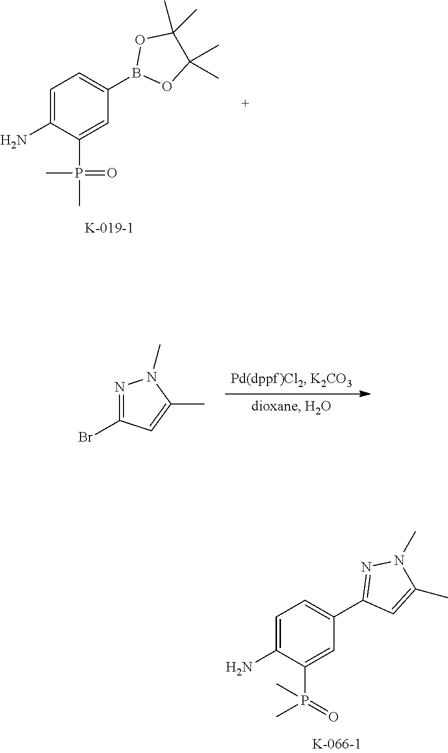

Add compound K-019-1 (500 mg), 1,5-Dimethyl-3-bromo-1h-pyrazole (297 mg), K₂CO₃ (702 mg), Pd(dppf)Cl₂ (124 mg), 1,4-dioxane (10 mL) and water (2 mL) to the reaction flask, heat to 100° C. under nitrogen protection, and stir with heating for 12 hr. The reaction was complete as monitored by LCMS, and then stopped. Add water (5 mL) into the reaction solution. And then the reaction solution was extracted with dichloromethane (3×10 mL); the organic phase was washed with saturated brine (3×5 mL), dried over anhydrous sodium sulfate, separated and purified by column chromatography (dichloromethane:methanol=10:1), and the solvent was removed to obtain brown solid of desired product K-066-1 (400 mg), MS: 264.12 [M+H]⁺

### Step 3: Synthesis of Compound K-066-2

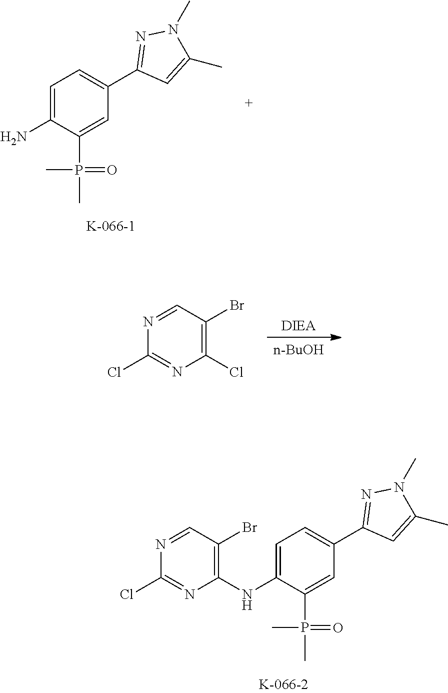

Add K-066-1 (400 mg), 5-Bromo-2,4-dichloropyrimidine (519 mg), DIEA (589 mg), n-BuOH (5 mL) to the reaction flask, heat to 120° C. and stir for 10 hr. The reaction was complete as monitored by LCMS. The reaction solution was cooled down to room temperature and filtered. The filter cake was dried to obtain the white solid K-066-2 (300 mg), MS: 454.01 [M+H]⁺

### Step 4: Synthesis of Compound K-066

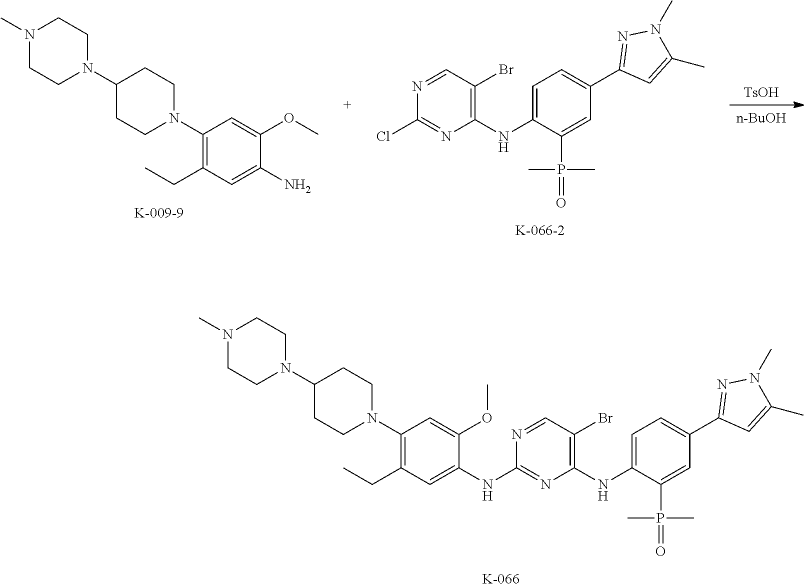

Add K-009-9 (73 mg), K-066-2 (100 mg), p-toluenesulfonic acid (57 mg) and n-BuOH (1 mL) to the reaction flask, and stir at 120° C. overnight. The reaction was complete as monitored by LCMS. The reaction solution was cooled down to room temperature, and spin-dried. Saturated Na₂CO₃ aqueous solution (10 mL) was added to the reaction solution. And then the solution was extracted with ethyl acetate (3×10 mL), dried over anhydrous sodium sulfate, and then filtered and spin-dried. The crude product was purified by preparative thin-layer chromatography (dichloromethane:methanol=10:1) to obtain white solid of purified K-066 (90 mg), MS: 750.29 [M+H]⁺

### Example 67: Synthesis of Compound K-067

### (2-((5-Bromo-2-((5-ethyl-2-methoxy-4-(4-(4-methylpiperazin-1-/l)piperidin-1-yl)phenyl)amino)pyrimidin-4-yl)amino)-5-(5,6-dimethylpyrazin-2-yl)phenyl)dimethylphosphine oxide

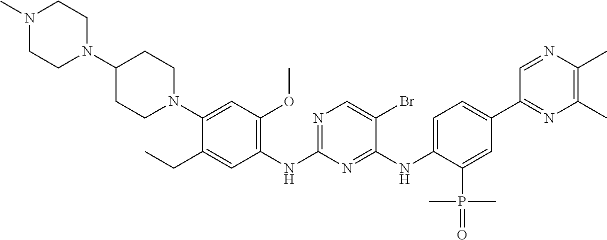

### Step 1: Synthesis of Compound K-067-1

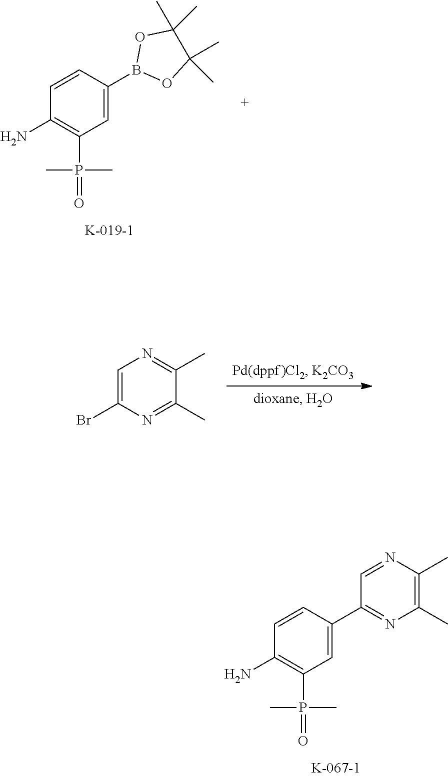

The compound K-067-1 was synthesized as the method described for compound K-019-2, that is compound K-019-1 reacted with 5-Bromo-2,3-dimethylpyrazine to produce compound K-067-1, MS: 276.12 [M+H]⁺

### Step 2: Synthesis of Compound K-067-2

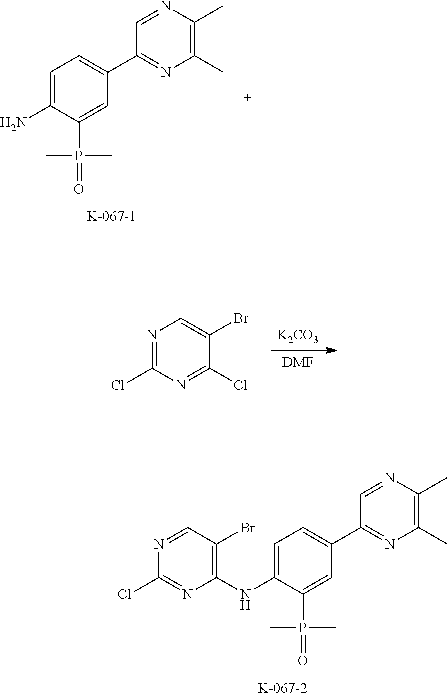

The compound K-067-2 was synthesized as the method described for compound K-018-5, that is 5-Bromo-2,4-dichloropyrimidine reacted with compound K-067-1 to produce compound K-067-2, MS: 466.01 [M+H]⁺

### Step 3: Synthesis of Compound K-067

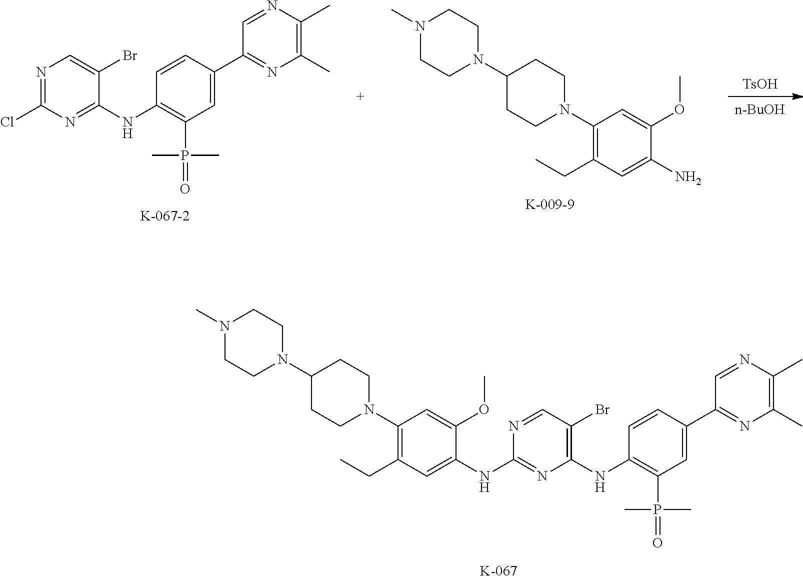

The compound K-067 was synthesized as the method described for compound K-018, that is compound K-009-9 reacted with compound K-067-2 to produce compound K-067, MS: 762.29 [M+H]⁺

### Example 68: Synthesis of Compound K-068

### (2-((5-Bromo-2-((5-ethyl-2-methoxy-4-(4-(4-methylpiperazin-1-yl)piperidin-1-yl)phenyl)amino)pyrimidin-4-yl)amino)-5-(2-isopopyl-2H-1,2,3-triazol-4-yl)phenyl)dimethylphosphine oxide

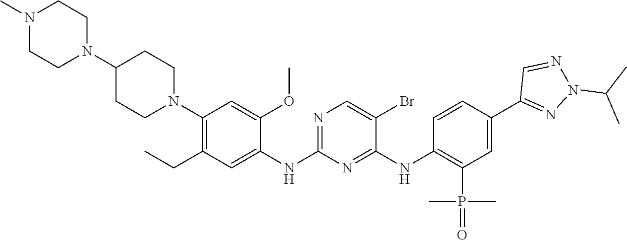 [SMILES: CCc1cc(Nc2ncc(Br)c(Nc3ccc(-c4cnn(C(C)C)n4)cc3P(C)(C)=O)n2)c(OC)cc1N1CCC(N2CCN(C)CC2)CC1]

### Step 1: Synthesisofcompound K-068-1

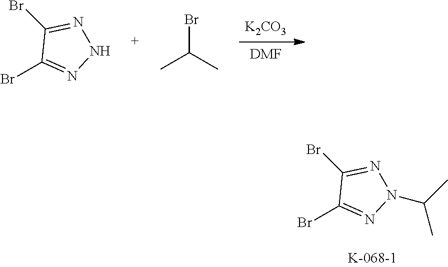

Add 4.5-Dibromo-2H-1,2,3-triazole (3.00 g), 2-Bromopropane (1.79 g), K2CO3 (2.74 g) and DMF (50 mL) to the reaction flask, raise temperature to 90° C., and stir with heating for 12 hr. The reaction solution was poured into water (100 mL), and extracted with dichloromelhane (3×40 mL); the organic phase was washed with saturated brine (3×30 mL), dried over anhydrous sodium sulfate, separated and purified by column chromatography (n-hexane:EA=10:1) to obtain the white solid of desired product, MS: 267.90 [M+H]⁺

### Step 2: Synthesis of Compound K-068-2

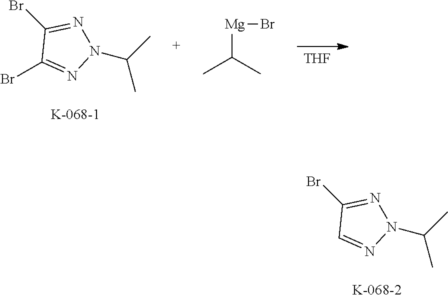

K-068-1 (300 mg) was dissolved in THF (3 mL), the solution was cooled down to −10° C. and isopropylmagnesium bromide (1.3 M, 0.94 mL) was slowly added dropwise. Keep the reaction temperature at ±5° C., and continue stirring for 30 min, then slowly add water (2 ml) dropwise and stir for 1 hr. The pH of reaction solution was adjusted to 1-2 with hydrochloric acid, extracted with ethyl acetate (3×30 ml); the organic phase was washed with saturated brine (3×30 mL), and dried over anhydrous sodium sulfate to obtain brown liquid of desired product compound K-068-2 (500 mg), MS: 189.99 [M+H]⁺

### Step 3: Synthesis of Compound K-068-3

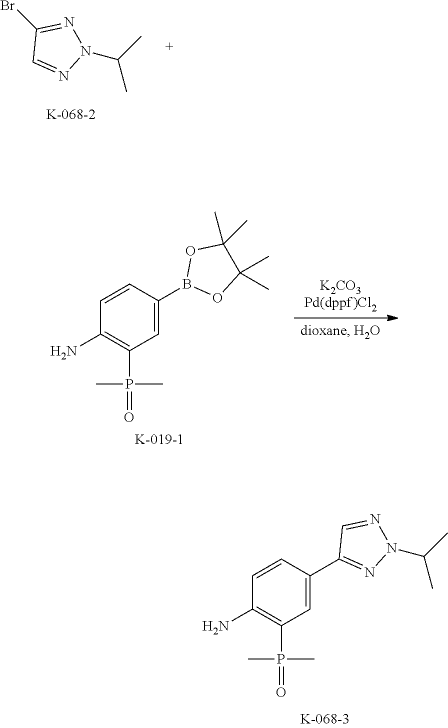

The compound K-068-3 was synthesized as the method described for compound K-019-2 using compound K-068-2 instead of raw compound 2-Bromothiazole. For compound K-068-3, MS: 279.13 [M+H]⁺

### Step 4: Synthesis of Compound K-068-4

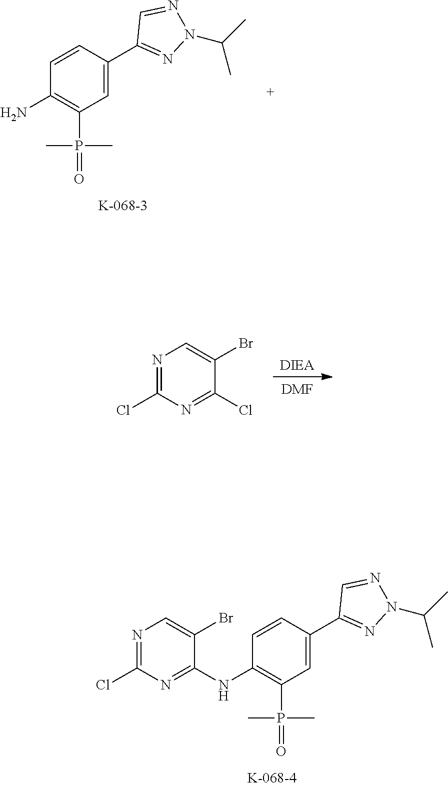

The compound K-068-4 was synthesized as the method described for compound K-018-5 using compound K-068-3 instead of raw compound K-018-4. For compound K-068-4, MS: 469.70 [M+H]⁺

### Step 5: Synthesis of Compound K-068

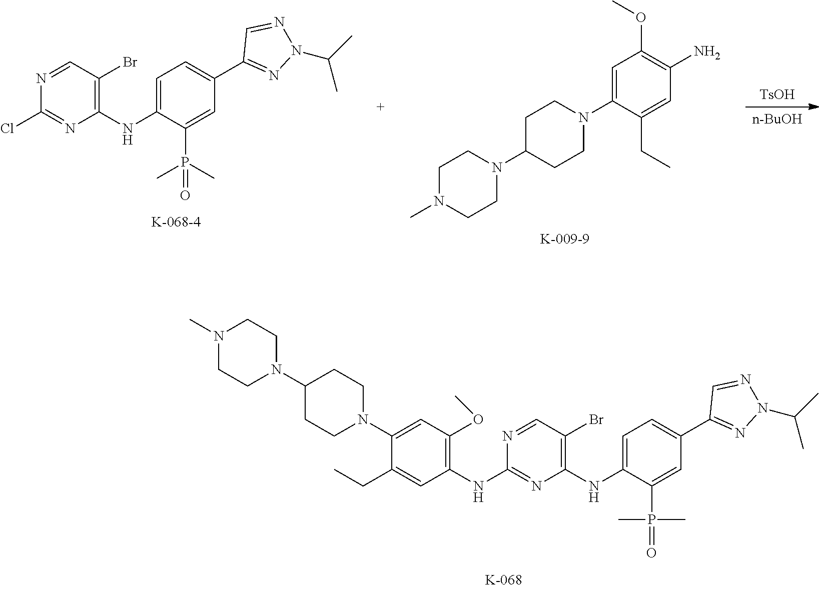

The compound K-068 was synthesized as the method described for compound K-018 using compound K-009-9 instead of raw compound K-018-9, compound K-068-4 instead of raw compound K-018-5. For compound K-068, MS: 765.30 [M+H]⁺

### Example 69: Synthesis of Compound K-069

### (2-((5-Bromo-2-((5-ethyl-2-methoxy-4-(4-(4-methylpiperazin-1-yl)piperidin-1-yl)phenyl)amino)pyrimidin-4-yl)amino)-5-(4-methyl-1H-pyrazol-1-yl)phenyl)dimethylphosphine oxide

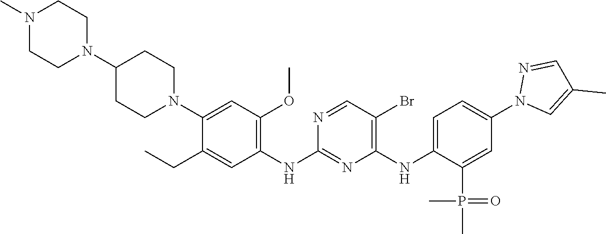

### Step 1: Synthesis of Compound K-069-1

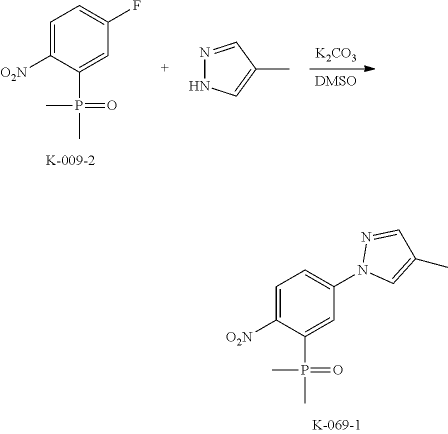

The compound K-069-1 was synthesized as the method described for compound K-009-3 using compound 4-Methylpyrazole instead of raw compound K-009-2A. For compound K-069-1, MS: 280.08 [M+H]⁺

### Step 2: Synthesis of Compound K-069-2

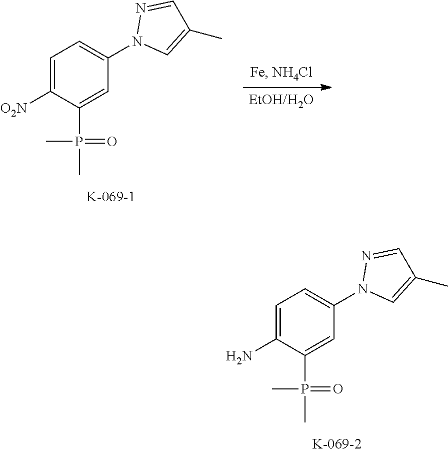

The compound K-069-2 was synthesized as the method described for compound K-009-4 using compound K-069-1 instead of raw compound K-009-3. For compound K-069-2, MS: 250.10 [M+H]⁺

### Step 4: Synthesis of Compound K-069-3

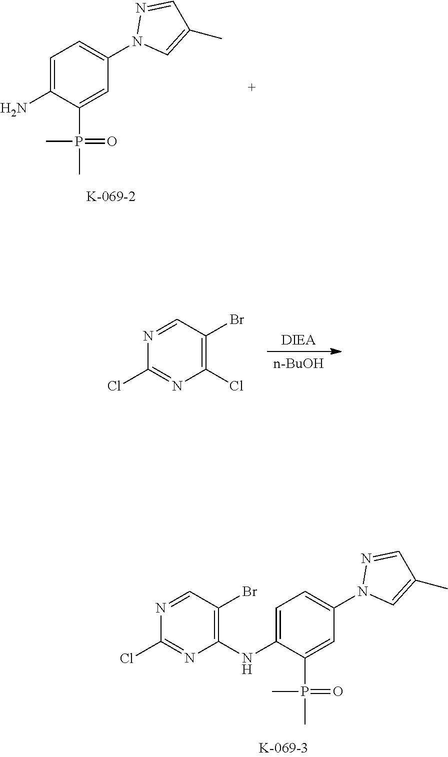

The compound K-069-3 was synthesized as the method described for compound K-009-5 using compound K-069-2 instead of raw compound K-009-4. For compound K-069-3, MS: 440.00 [M+H]⁺

### Step 5: Synthesis of Compound K-069

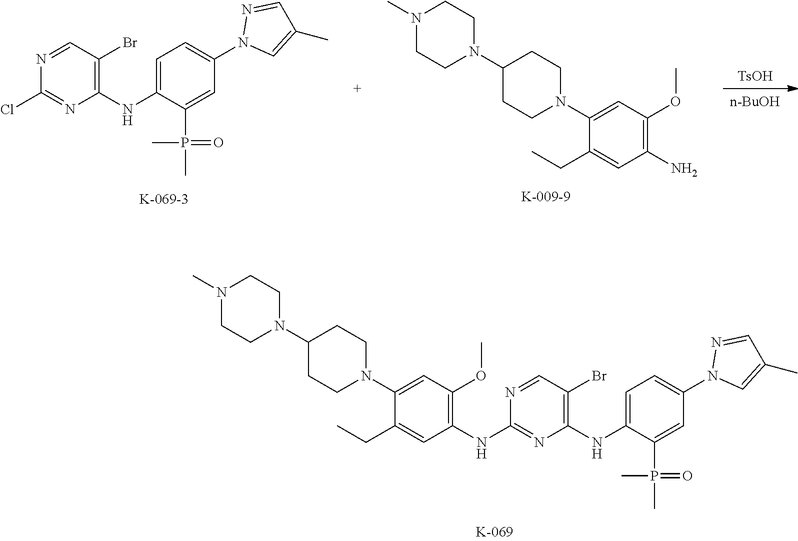

The compound K-069 was synthesized as the method described for compound K-009 using compound K-069-3 instead of raw compound K-009-5. For compound K-069, MS: 736.28 [M+H]⁺

### Example 70: Synthesis of Compound K-070

### (2-((5-Bromo-2-((5-ethyl-2-methoxy-4-(4-(4-methylpiperazin-1-yl)piperidin-1-yl)phenyl)amino)pyrimidin-4-yl)amino)-5-(6-methylpyridin-3-yl)phenyl)dimethylphosphine oxide

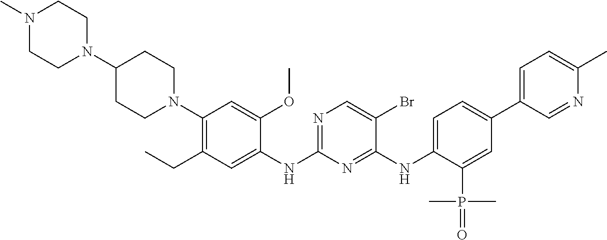

### Step 1: Synthesis of Compound K-070-1

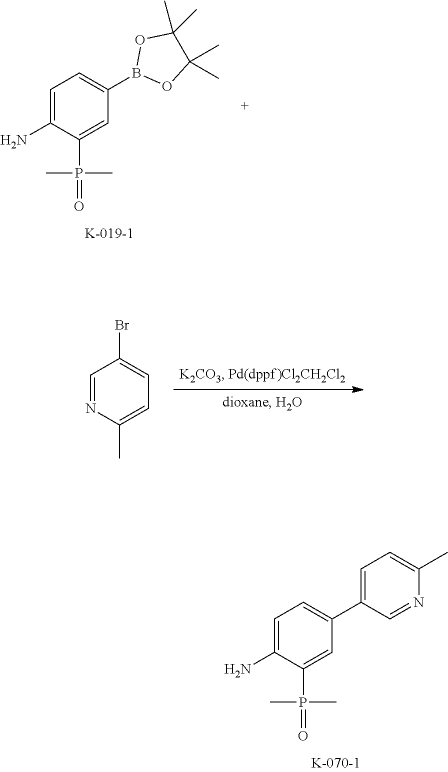

The compound K-070-1 was synthesized as the method described for compound K-019-2, that is compound K-019-1 reacted with 5-Bromo-2-methylpyridine (175 mg) to produce a solid powder of compound K-070-1 (227 mg), MS: 261.11 [M+H]⁺.

### Step 2: Synthesis of Compound K-070-2

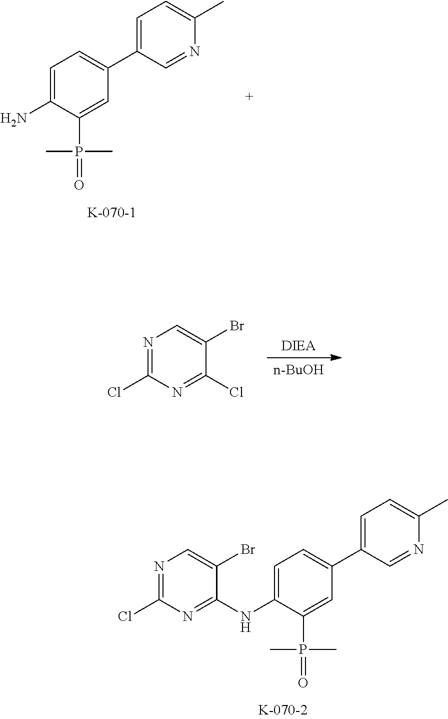

The compound K-070-2 was synthesized as the method described for compound K-018-5, that is 5-Bromo-2,4-dichloropyrimidine reacted with compound K-070-1 (227 mg) to produce a solid powder of compound K-070-2 (300 mg), MS: 451.00 [M+H]⁺.

### Step 3: Synthesis of Compound K-070

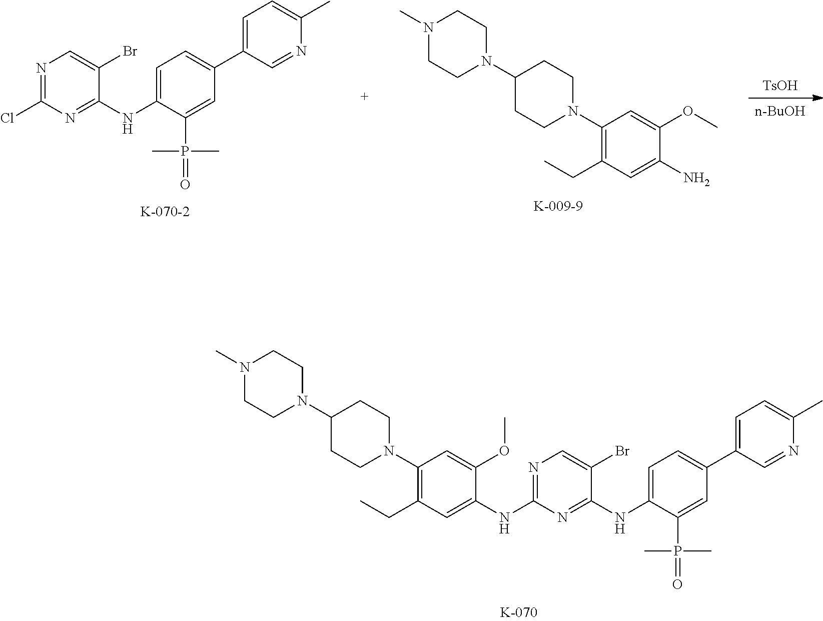

The compound K-070 was synthesized as the method described for compound K-018, that is compound K-009-9 reacted with compound K-070-2 (300 mg) to produce a brown powder of compound K-070 (38.5 mg), MS: 747.28 [M+H]⁺.

### Example 71: Synthesis of Compound K-071

(2-((5-Bromo-2-((5-ethyl-2-methoxy-4-(4-(4-methylpiperazin-1-yl)piperidin-1-yl)phenyl)amino)pyrimidin-4-yl)amino)-5-(5-methylpyridin-2-yl)phenyl)dimethylphosphine oxide

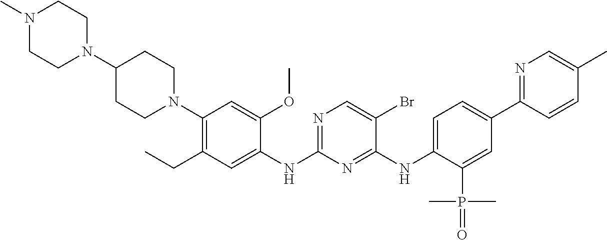

### Step 1: Synthesis of Compound K-071-1

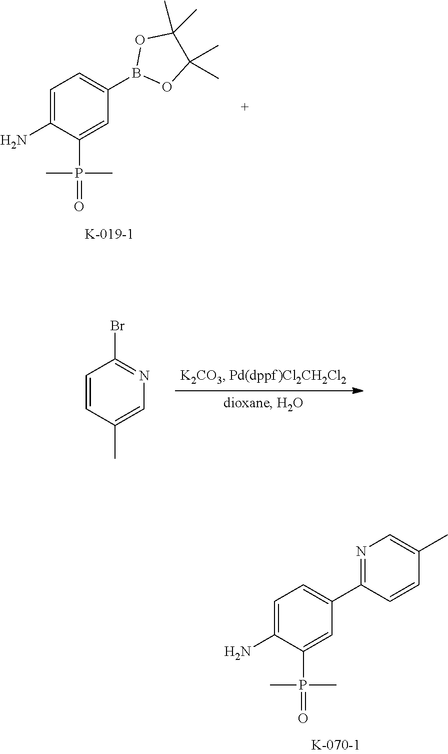

The compound K-070-1 was synthesized as the method described for compound K-019-2, that is compound K-019-1 reacted with 2-Bromo-5-methylpyridine (175 mg) to produce a solid powder of compound K-071-1 (225 mg), MS: 261.11 [M+H]⁺.

### Step 2: Synthesis of Compound K-071-2

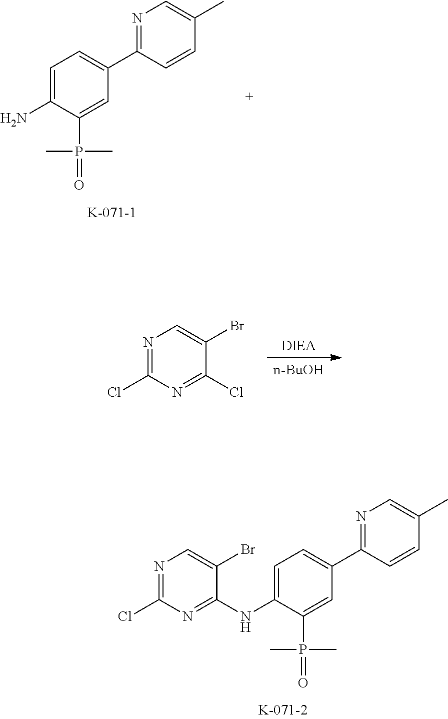

The compound K-071-2 was synthesized as the method described for compound K-018-5, that is 5-Bromo-2,4-dichloropyrimidine reacted with compound K-071-1 (225 mg) to produce a solid powder of compound K-071-2 (300 mg), MS: 451.00 [M+H]⁺.

### Step 3: Synthesis of Compound K-071

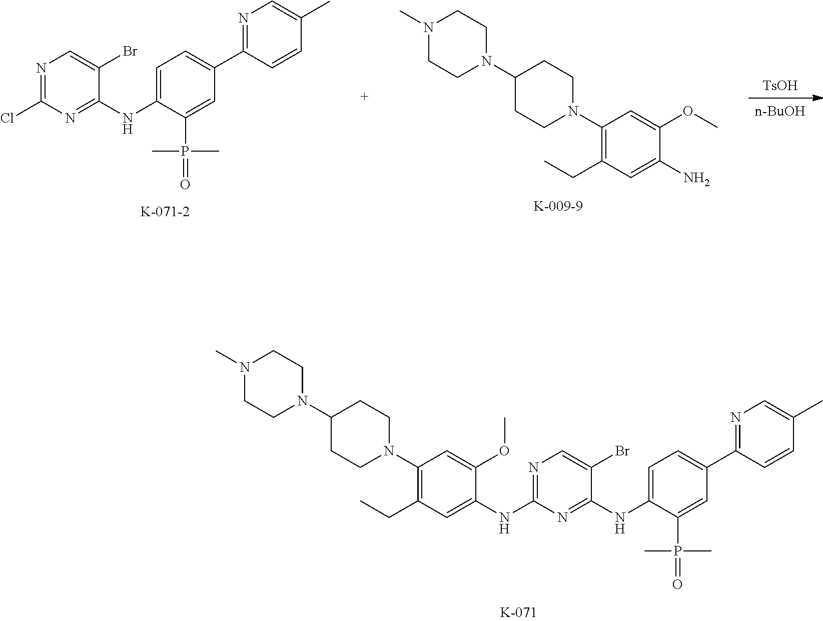

The compound K-071 was synthesized as the method described for compound K-018, that is compound K-009-9 reacted with compound K-071-2 (300 mg) to produce a brown powder of compound K-071 (54.5 mg), MS: 747.28 [M+H]⁺.

### Example 72: Synthesis of Compound K-072

### (2-((5-Bromo-2-((5-ethyl-2-methoxy-4-(4-(4-methylpiperazin-1-yl)piperidin-1-yl)phenyl)amino)pyrimidin-4-yl)amino)-5-(5-methoxythiophen-2-yl)phenyl)dimethylphosphine oxide

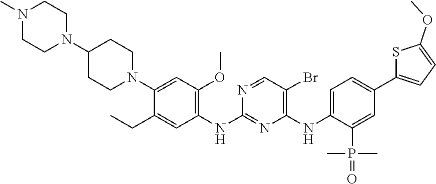 [SMILES: CCc1cc(Nc2ncc(Br)c(Nc3ccc(-c4ccc(OC)s4)cc3P(C)(C)=O)n2)c(OC)cc1N1CCC(N2CCN(C)CC2)CC1]

### Step 1: Synthesis of Compound K-072-1

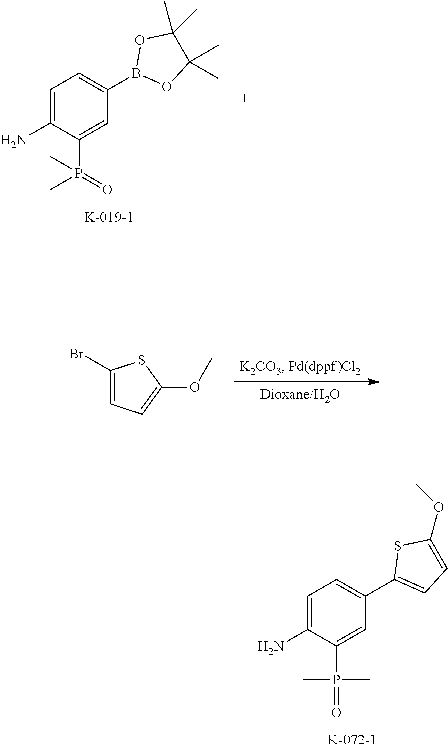

Compound K-019-1 (500 mg), 2-Bromo-5-methoxy thiophene (327 mg), Pd(dppf)Cl₂ (276 mg) and K₂CO₃ (351 mg) were added to the reaction flask, and then added to dioxane (8 mL) and water (2 mL) successively. The solution was heated to 100° C. and stirred for 12 hr. The reaction was complete as monitored by LCMS, and then stopped. The reaction solution was poured into water (5 mL), and extracted with dichloromethane (3×10 mL); the organic phase was washed with saturated brine (3×10 mL), dried over anhydrous sodium sulfate, extracted, and then separated and purified by column chromatography (dichloromethane:methanol=10:1), followed by concentration to obtain the brown solid of desired compound K-072 (400 mg), MS: 282.06 [M+H]⁺

### Step 2: Synthesis of Compound K-072-2

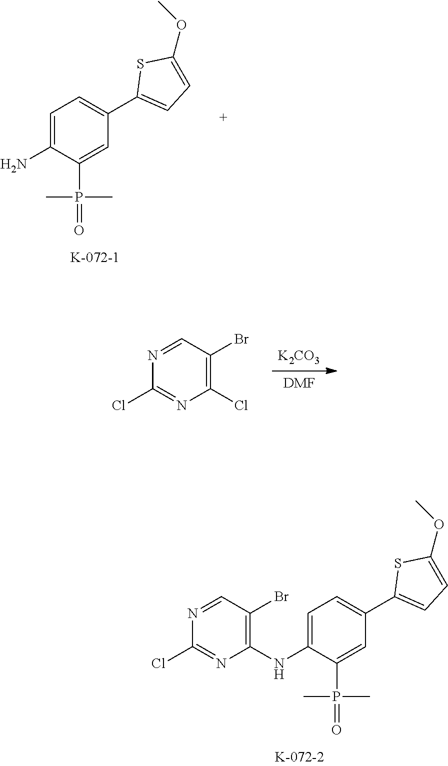

Add compound K-072-1 (180 mg), 5-Bromo-2,4-dichloropyrimidine (291 mg), K₂CO₃ (176 mg) and DMF (20 mL) to the reaction flask, heat to 90° C., and stir with heating for 4 hr. The reaction was complete as monitored by LCMS, and then stopped. The reaction solution was poured into water (50 mL), and extracted with ethyl acetate (3×50 mL); the organic phase was washed with saturated brine (3×30 mL), dried over anhydrous sodium sulfate, separated and purified by column chromatography (dichloromethane:methanol=13:1). The solvent was removed to obtain the yellow solid of desired product compound K-072-2 (180 mg), MS: 471.96 [M+H]⁺

### Step 3: Synthesis of Compound K-072

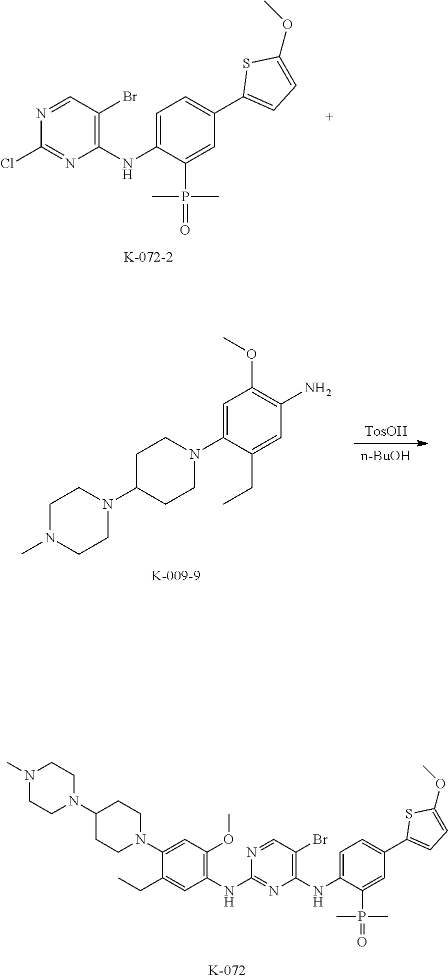

Add compounds K-072-2 (120 mg), K-009-9 (101 mg), p-toluenesulfonic acid (34 mg) and n-BuOH (2 mL) to the reaction flask, heat to 120° C. and stir for 12 hr. The reaction was complete as monitored by LCMS, and then stopped. The reaction solution was poured into sodium carbonate solution (5 mL), and extracted with dichloromethane (3×10 mL); the organic phase was washed with saturated brine (3×10 mL), and dried over anhydrous sodium sulfate; after positive phase separation (MeOH:H₂O, 0%-30%), the resulting product was extracted, and then separated and purified by column chromatography (dichloromethane:methanol=10:1), concentrated to obtain the off-white solid of compound K-072 (12 mg), MS: 768.24 [M+H]⁺

### Example 73: Synthesis of Compound K-073

### (2-((5-Bromo-2-((5-ethyl-2-methoxy-4-(4-(4-methylpiperazin-1-yl)piperidin-1-yl)phenyl)amino)pyrimidin-4-yl)amino)-5-(4-chlorothiazol-2-yl)phenyl)dimethylphosphine oxide

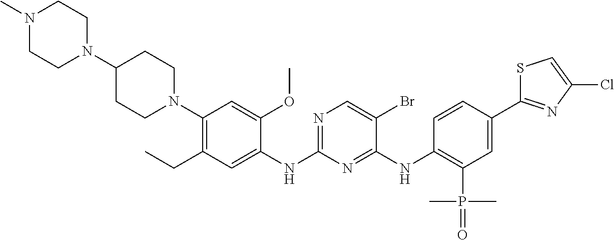 [SMILES: CCc1cc(Nc2ncc(Br)c(Nc3ccc(-c4nc(Cl)cs4)cc3P(C)(C)=O)n2)c(OC)cc1N1CCC(N2CCN(C)CC2)CC1]

### Step 2: Synthesis of Compound K-073-1

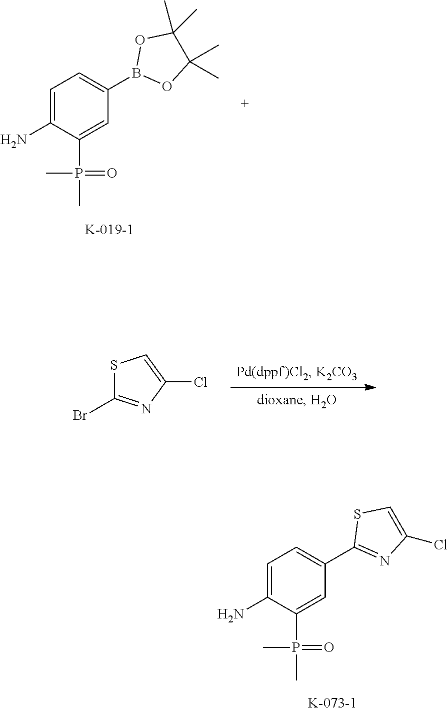 [SMILES: CC1(C)OB(c2ccc(N)c(P(C)(C)=O)c2)OC1(C)C ; Clc1csc(Br)n1 ; "Pd(dppf)Cl2, K2CO3" ; "dioxane, H2O" ; CP(C)(=O)c1cc(-c2nc(Cl)cs2)ccc1N]

Add compound K-019-1 (2.00 g), 2-Bromo-4-chlorothiazole (1.34 g), K₂CO₃ (2.81 g), Pd(dppf)Cl₂ (496 mg), 1,4-dioxane (20 mL) and water (4 mL) to the reaction flask; heat to 100° C. under nitrogen protection and stir for 12 hr. The reaction was complete as monitored by LCMS, and then stopped. After adding water (5 mL), the reaction solution was extracted with dichloromethane (3×10 mL); the organic phase was washed with saturated brine (3×5 mL), dried over anhydrous sodium sulfate, separated and purified by column chromatography (dichloromethane:methanol=10:1). The solvent was removed to obtain the brown solid of desired compound K-073-1 (1.5 g), MS: 287 [M+H]⁺

### Step 3: Synthesis of Compound K-073-2

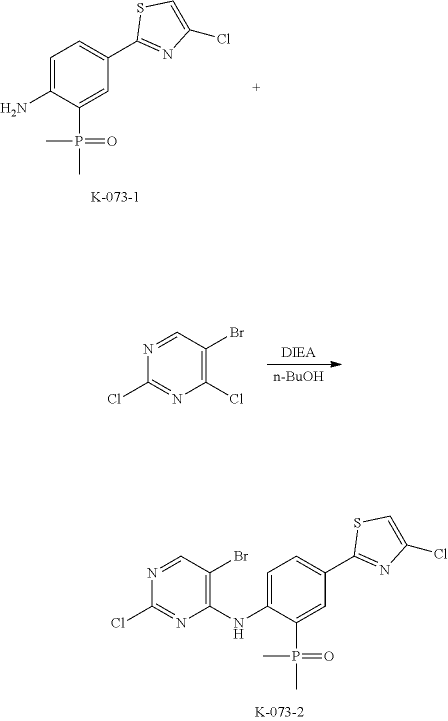

Add K-073-1 (600 mg), 5-Bromo-2,4-dichloropyrimidine (715 mg), DIEA (1.35 g) and n-BuOH (10 mL) to the reaction flask, heat to 120° C. and stir for 10 hr. The reaction was complete as monitored by LCMS. The reaction solution was cooled down to room temperature, and filtered. The filter cake was dried to obtain the yellowish solid K-073-2 (300 mg), MS: 476.90 [M+H]⁺

### Step 4: Synthesis of Compound K-07

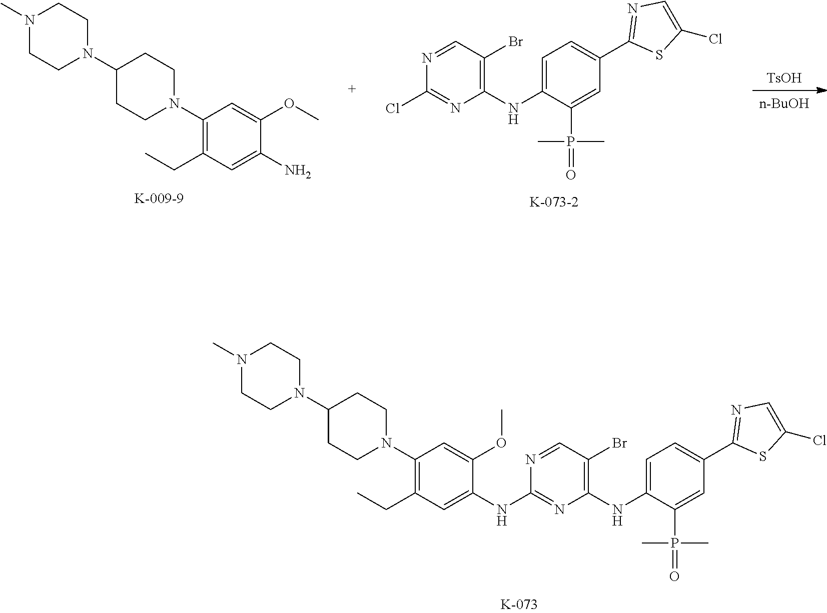

Add compound K-009-9 (100 mg), K-073-2 (70 mg), p-toluenesulfonic acid (54 mg) and n-BuOH (1 mL) to the reaction flask, stir at 120° C. overnight. The reaction was complete as monitored by LCMS. The reaction solution was cooled down to room temperature, and spin-dried. Saturated Na₂CO₃ aqueous solution (10 mL) was added to the reaction solution. And then the solution was extracted with dichloromethane (3×10 mL), dried over anhydrous sodium sulfate, filtered and spin-dried. The crude product was purified by PTLC (dichloromethane:methanol=10:1) to obtain white solid of purified K-073 (87 mg), MS: 773.18 [M+H]⁺

### Example 74: Synthesis of Compound K-074

### (2-((5-Bromo-2-((5-ethyl-2-methoxy-4-(4-(4-methylpiperazin-1-yl)piperidin-1-yl)phenyl)amino)pyrimidin-4-yl)amino)-5-(5-methoxypyridin-2-yl)phenyl)dimethylphosphine oxide

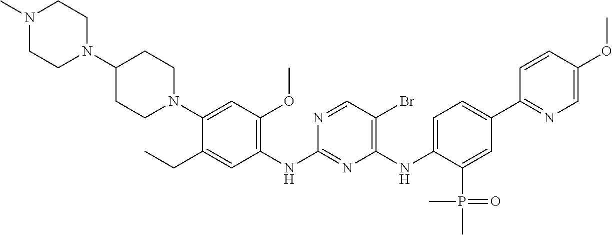

### Step 1: Synthesis of Compound K-074-1

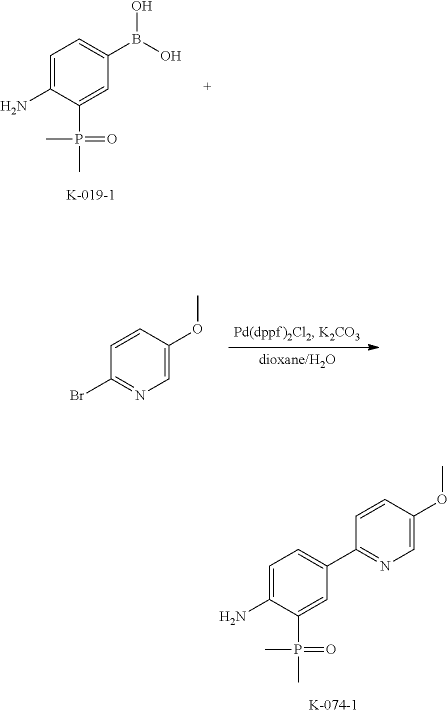

The compound K-074-1 was synthesized as the method described for compound K-019-2 using compound 2-Bromo-5-methoxypyridine instead of raw compound 2-Bromothiazole. For compound K-074-1, MS: 277.10 [M+H]⁺

### Step 3: Synthesis of Compound K-074-2

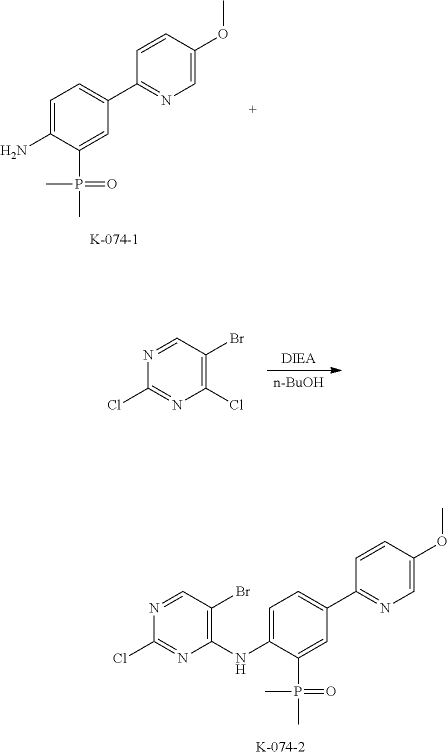

The compound K-074-2 was synthesized as the method described for compound K-019-3 using compound K-074-1 instead of raw compound K-019-2. For compound K-074-2, MS: 467.10 [M+H]⁺

### Step 3: Synthesis of Compound K-074

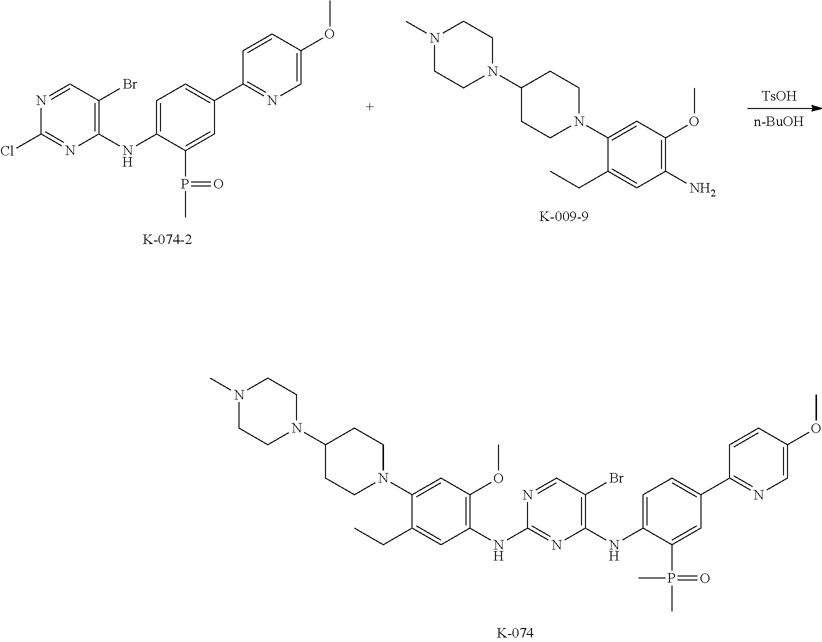

The compound K-074 was synthesized as the method described for compound K-019 using compound K-074-2 instead of raw material K-019-3, compound K-009-9 instead of raw material K-019-8. For compound K-074, MS: 763.28 [M+H]⁺

### Example 75: Synthesis of Compound K-075

### (6-((5-Bromo-2-((5-methoxy-2-methyl-4-(4-(4-methylpiperazin-1-yl)piperidin-1-yl)phenyl)amino)pyrimidin-4-yl)amino)-2-fluoro-3-(4-methoxy-1H-pyrazol-1-yl)phenyl)dimethylphosphine oxide

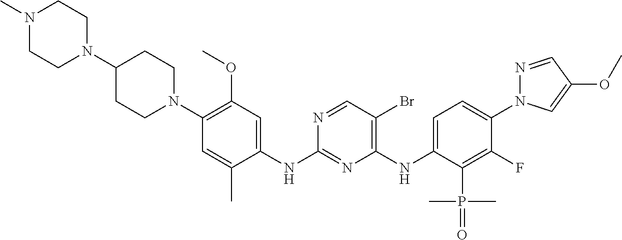

### Step 1: Synthesis of Compound K-075-2

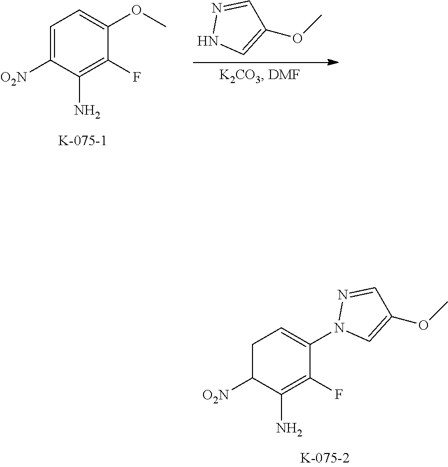

Dissolve K-075-1 (1.0 g), 4-Methoxy-1H-pyrazole (563 mg) in DMF (8 mL), add K₂CO₃ (1.19 mg) to the solution at room temperature; the gas in reactor was replaced with nitrogen for three times. After well stirring, the solution was transferred to an oil bath, heated to 100° C., and stirred at 100° C. for 16 hr. The reaction was complete as monitored by LCMS. The reaction solution was cooled down to room temperature, poured into water, and extracted with ethyl acetate (3×50 mL); the organic phase was washed with saturated brine (3×30 mL), dried over anhydrous sodium sulfate, separated and purified by column chromatography (n-hexane:ethyl acetate=0%-80%). The solvent was removed to obtain the desired product K-075-2 (1.2 g), MS: 253.07 [M+H]⁺

### Step 2: Synthesis of Compound K-075-3

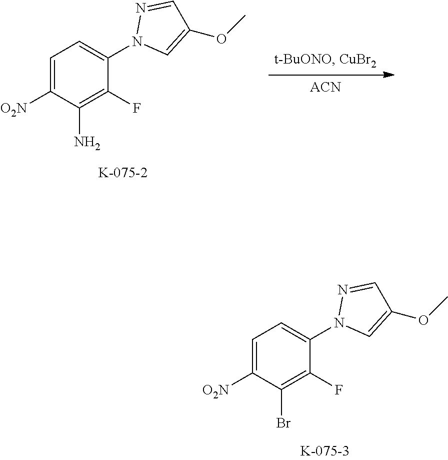

tBuONO (245 mg) and CuBr₂ (425 mg) were dissolved in 10 mL acetonitrile in a reactor equipped with a stir bar and nitrogen protector. After heating to 80° C., K-075-2 (0.4 g) was dissolved in ACN (10 mL), and solution of which was added dropwise to above-mentioned system, the gas in reactor was replaced with nitrogen for three times. After well stirring, the solution was stirred at 80° C. for 1 hr. The reaction was complete as monitored by TLC. The reaction solution was poured into water, and extracted with EtOAc (50 ml) for three times; the organic phase was dried over anhydrous sodium sulfate; after filtering off anhydrous sodium sulfate, the solution was concentrated by rotary evaporator. Silica gel was added to the concentrated organic phase to mix the sample, and then which was separated and purified by PE:EtOAc column with gradient from 0% to 40%. After that, it was concentrated and dried to obtain the purple solid K-075-3 (0.45 g).

### Step 3: Synthesis of Compound K-075-4

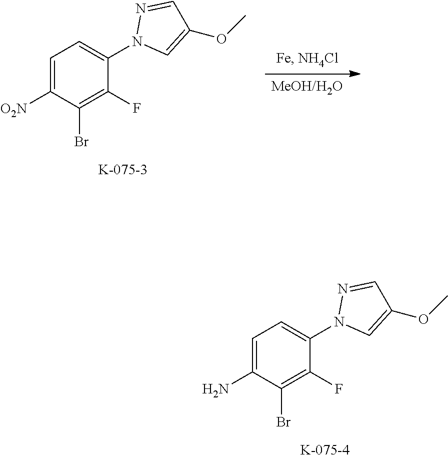

Dissolve K-075-3 (45 mg) in MeOH (8 ml) and H₂O (8 mL), add iron powder (397 mg) and NH₄Cl (152 mg) at room temperature; the gas in reactor was replaced with nitrogen for three times. After well stirring, the solution was transferred to an oil bath, heated to 80° C., and stirred at 80° C. for 3 hr. The reaction was complete as monitored by LCMS. The reaction solution was cooled down to room temperature, poured into water, and extracted with DCM/MeOH-10:1 (3×50 mL); the organic phase was washed with saturated brine (3×30 mL), dried over anhydrous sodium sulfate, separated and purified by column chromatography (DCM/MeOH=0%-10% h). The solvent was removed to obtain desired product K-075-4 (350 mg), MS: 285.99 [M+H]⁺

### Step 4: Synthesis of Compound K-075-5

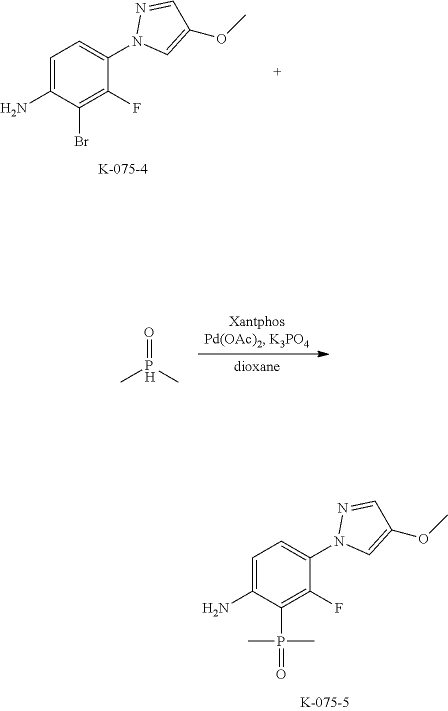

Add compound K-075-4 (0.4 g), dimethylphosphine oxide (143 mg), K₃PO₄ (389 mg), Pd(OAc)₂ (27 mg), Xantphos (70 g), 1,4-dioxane (10 ml) to the reaction flask, heat to 120° C. under nitrogen protection, and stir with heating for 16 hr. The reaction was complete as monitored by LCMS, and then stopped. The reaction solution was poured into water (5 mL), and extracted with dichloromethane (3×50 mL); the organic phase was washed with saturated brine (3×30 mL), dried over anhydrous sodium sulfate, separated and purified by column chromatography (dichloromethane:methanol=15:1), then concentrated and dried to obtain yellow solid of desired product K-075-5 (150 mg), MS: 284.09 [M+H]⁺

### Step 5: Synthesis of Compound K-075-6

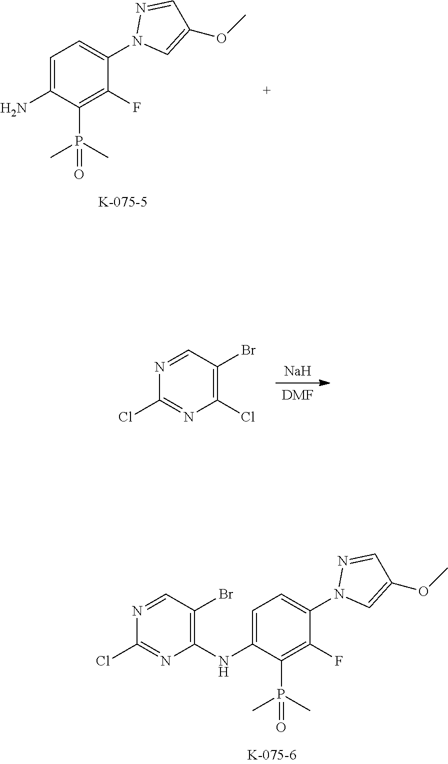

Add compound K-075-5 (150 mg), NaH (42 mg) and DMF (20 mL) to the reaction flask, and then add 5-Bromo-2,4-dichloropyrimidine (1.39 g), heat to 60° C., and stir with heating for 2 hr. The reaction was complete as monitored by LCMS, and then stopped. The reaction solution was poured into water (20 ml) solution, and extracted with ethyl acetate (3×20 mL); the organic phase was washed with saturated brine (3×30 ml), dried over anhydrous sodium sulfate, separated and purified by column chromatography (dichloromethane:methanol=13:1), and the solvent was removed to obtain brown solid of desired product compound K-075-6 (160 mg), MS: 473.98 [M+H]⁺

### Step 6: Synthesis of Compound K-075

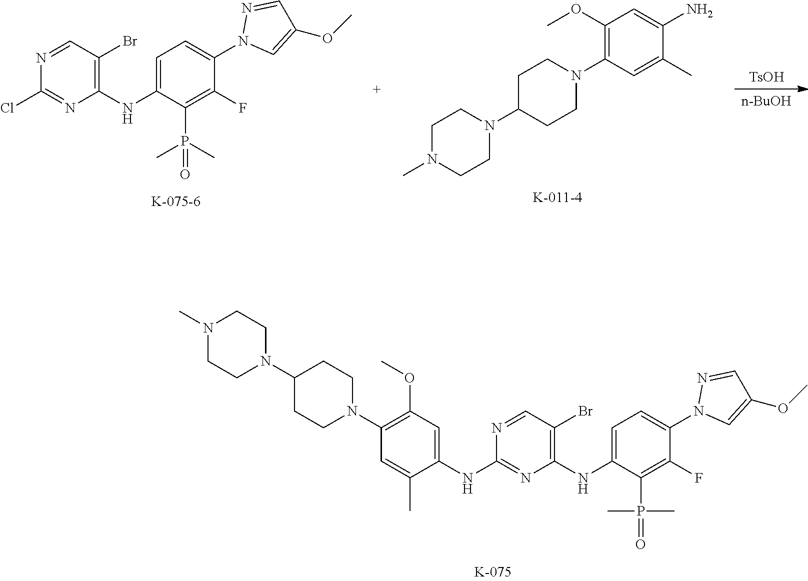

Add compounds K-075-6 (100 mg, purity: 30%). K-011-4 (20 mg), p-toluenesulfonic acid (10 mg) and n-butanol (2 mL) to the reaction flask, heat to 120° C. and stir for 12 hr. The reaction was complete as monitored by LCMS, and then stopped. The reaction solution was poured into sodium carbonate solution (5 mL), and extracted with dichloromethane (3×10 ml); the organic phase was washed with saturated brine (3×10 mL), and dried over anhydrous sodium sulfate; after negative phase separation (MeOH:H₂O, 0%/6-30%), the resulting product was extracted, and then separated and purified by column chromatography (dichloromethane:methanol=10:1), followed by concentration to obtain the white solid of compound K-075 (II mg), MS: 756.25 [M+H]⁺

### Example 76: Synthesis of Compound K-076

### (2-((5-Bromo-2-((2-methoxy-5-methyl-4-(4-(4-methylpiperazin-1-yl)piperidin-1-yl)phenyl))amino)pyrimidin-4-yl)amino)-5-(4-methoxy-1H-pyrazol-1-yl)phenyl)dimethylphosphine oxide

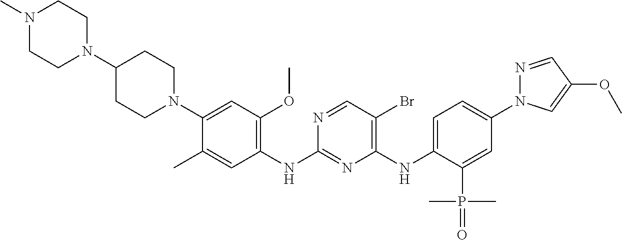

### Step 1: Synthesis of Compound K-076

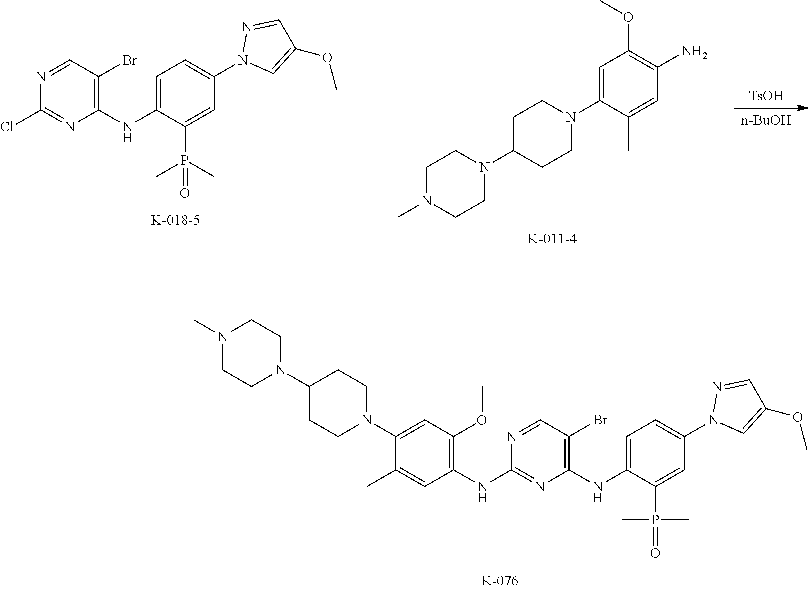

Add compounds K-018-5 (150 mg), K-011-4 (104 mg), p-toluenesulfonic acid (44 mg) and n-butanol (5 mL) to the reaction flask, heat to 120° C. and stir for 12 hr. The reaction was complete as monitored by LCMS, and then stopped. The reaction solution was poured into sodium carbonate solution (5 mL), and extracted with dichloromethane (3×10 mL); the organic phase was washed with saturated brine (3×10 mL), and dried over anhydrous sodium sulfate; after positive phase separation (MeOH:H₂O, 0%-30%), the resulting product was extracted, and then separated and purified by column chromatography (dichloromethane:methanol=10:1), followed by concentration to obtain the off-white solid of compound K-076 (54 mg), MS: 738.26 [M+H]⁺

### Example 77: Synthesis of Compound K-077

### (6-((5-Bromo-2-((2-methoxy-5-methyl-4-(4-(4-methylpiperazin-1-yl)piperidin-1-yl)phenyl)amino)pyrimidin-4-yl)amino)-2-fluoro-3-(4-fluoro-1H-pyrazol-1-yl)phenyl)dimethylphosphine oxide

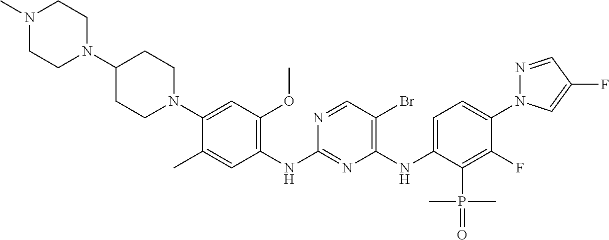 [SMILES: COc1cc(N2CCC(N3CCN(C)CC3)CC2)c(C)cc1Nc1ncc(Br)c(Nc2ccc(-n3cc(F)cn3)c(F)c2P(C)(C)=O)n1]

### Step 1: Synthesis of Compound K-077-2

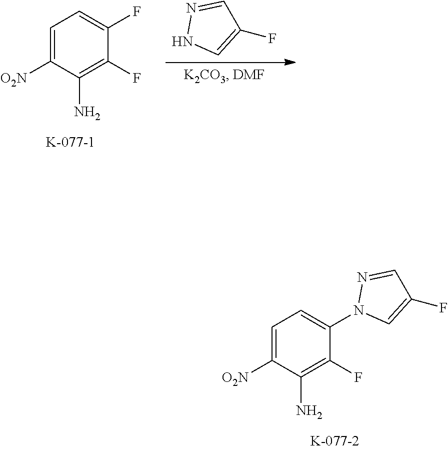

Dissolve K-077-1 (1.0 g) and 4-Fluoro-1H-pyrazole (494 mg) in DMSO (8 mL), add K₂CO₃ (1.19 mg) at room temperature; the gas in reactor was replaced with nitrogen for three times. After well stirring, the solution was transferred to an oil bath, heated to 100° C., and stirred under 100° C. for 16 hr. The reaction was complete as monitored by LCMS. The reaction solution was cooled down to room temperature, poured into water, extracted with ethyl acetate (3×50 mL), the organic phase was washed with saturated brine (3×30 mL), dried over anhydrous sodium sulfate, and purified by column chromatography (n-hexane:ethyl acetate=0%-80%). The solvent was removed to afford the desired product K-077-2 (1.21 g), MS: 241.05 [M+H]⁺

### Step 2: Synthesis of Compound K-077-3

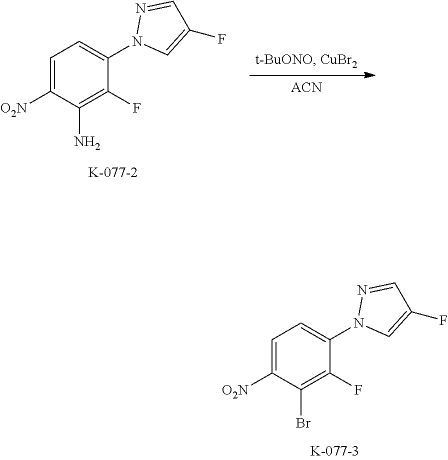

tBuONO (368 mg) and CuBr₂ (637.63 mg, 2.85 mmol) were dissolved in 10 mL acetonitrile in a reactor equipped with a stir bar and nitrogen protector. After heating to 80° C., K-077-2 (I g) was dissolved in ACN (10 mL), and solution of which was added dropwise to above-mentioned system, the gas in reactor was replaced with nitrogen for three times. After well stirring, the solution was stirred at 80° C. for 1 hr. The reaction was complete as monitored by TLC. The reaction solution was poured into water, and extracted with EtOAc (50 mL) for three times; the organic phase was dried over anhydrous sodium sulfate; after filtering off anhydrous sodium sulfate, the solution was concentrated by rotary evaporator. Silica gel was added to the concentrated organic phase to mix the sample, and then which was separated and purified by PE:EtOAc column with gradient from 0% to 40%. After that, it was concentrated and dried to obtain the purple solid K-077-3 (800 mg).

### Step 3: Synthesis of Compound K-077-4

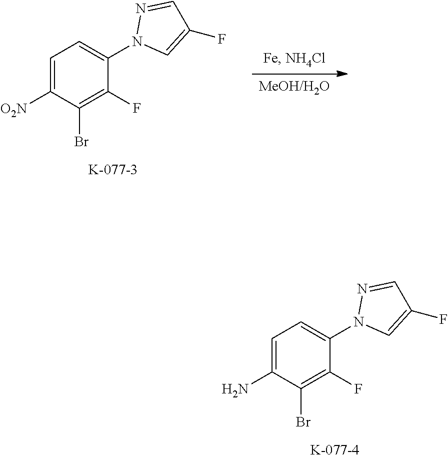

Dissolve K-077-3 (800 mg) in MeOH (8 mL) and H₂O (8 mL), add iron powder (706 mg) and NH₄Cl (270 mg) at room temperature; the gas in reactor was replaced with nitrogen for three times. After well stirring, the solution was transferred to an oil bath, heated to 80° C., and stirred at 80° C. for 3 hr. The reaction was complete as monitored by LCMS. The reaction solution was cooled down to room temperature, poured into water, and extracted with DCM/MeOH-10:1 (3×50 mL); the organic phase was washed with saturated brine (3×30 mL), dried over anhydrous sodium sulfate, separated and purified by column chromatography (DCM/MeOH=0%-10%). The solvent was removed to obtain desired product K-077-4 (700 mg), MS: 273.97 [M+H]⁺

### Step 4: Synthesis of Compound K-077-5

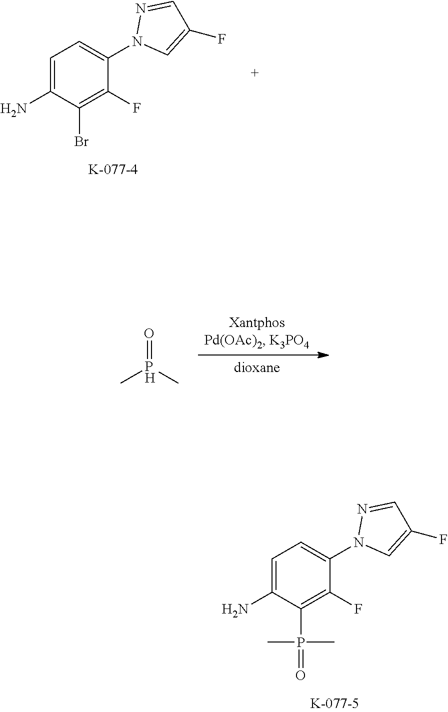

Add compound K-077-4 (0.4 g), dimethylphosphine oxide (170 mg), K₃PO₄ (464 mg), Pd(OAc)₂ (32 mg). Xantphos (84 g), 1,4-dioxane (10 mL) to the reaction flask, heat to 120° C. under nitrogen protection, and stir with heating for 16 hr. The reaction was complete as monitored by LCMS, and then stopped. The reaction solution was poured into water (50 mL), and extracted with dichloromethane (3×50 mL); the organic phase was washed with saturated brine (3×30 ml), dried over anhydrous sodium sulfate, separated and purified by column chromatography (dichloromethane:methanol=15:1), then concentrated and dried to obtain the yellow solid of desired product K-077-5 (150 mg), MS: 272.07 [M+H]⁺

### Step 5: Synthesis of Compound K-077-6

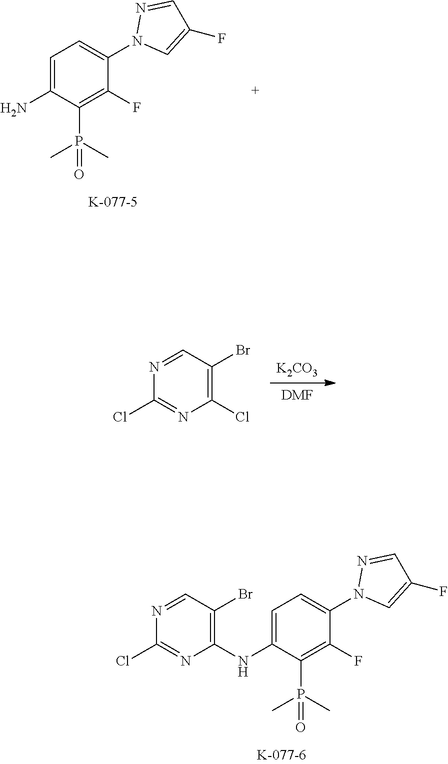

Add compound K-077-5 (150 mg), 5-Bromo-2,4-dichloropyrimidine (1.39 g), K₂CO₃ (141 mg) and DMF (20 mL) to the reaction flask, heat to 90° C., and stir with heating for 4 hr. The reaction was complete as monitored by LCMS, and then stopped. The reaction solution was poured into water (50 mL), and extracted with ethyl acetate (3×50 mL); the organic phase was washed with saturated brine (3×30 mL), dried over anhydrous sodium sulfate, separated and purified by column chromatography (dichloromethane:methanol=13:1), and the solvent was removed to obtain brown solid of desired product compound K-077-6 (260 mg), MS: 462.96 [M+H]⁺

### Step 6: Synthesis of Compound K-077

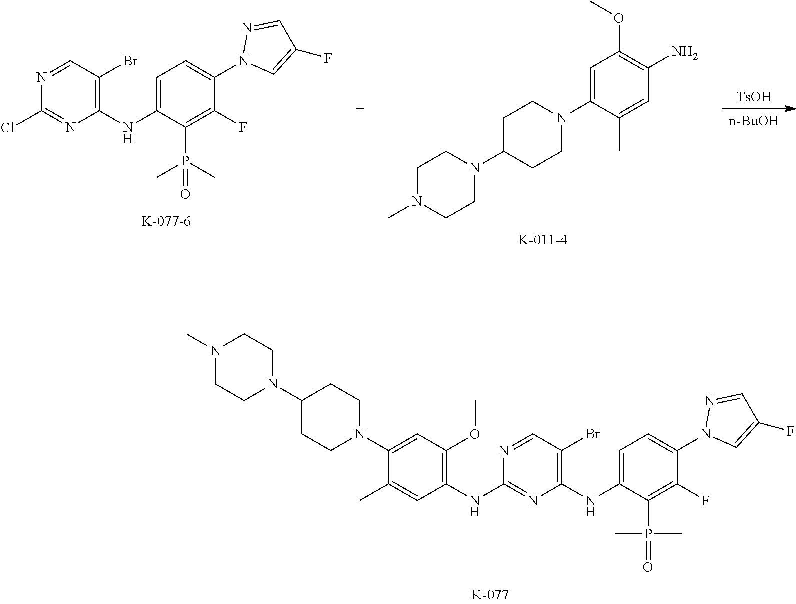

Add compounds K-077-6 (150 mg), K-011-4 (51 mg), p-toluenesulfonic acid (22 mg) and n-butanol (2 mL) to the reaction flask, heat to 120° C. and stir for 12 hr. The reaction was complete as monitored by LCMS, and then stopped. The reaction solution was poured into sodium carbonate solution (S ml), and extracted with dichloromethane (3×10 mL); the organic phase was washed with saturated brine (3×10 ml), and dried over anhydrous sodium sulfate; after negative phase separation (MeOH:H₂O, 0%-30%), the resulting product was extracted, and then separated and purified by column chromatography (dichloromethane:methanol=10:1), followed by concentration to obtain the off-white solid compound K-077 (20 mg), MS: 744.23 [M+H]⁺

### Example 78: Synthesis of Compound K-078

### (4-((5-Bromo-2-((5-ethyl-2-methoxy-4-(morpholinopiperidin-1-yl)phenyl)amino)pyrimidin-4-yl)amino)-2′-methyl-[1,1′-diphenyl]-3-yl)dimethylphosphine oxide

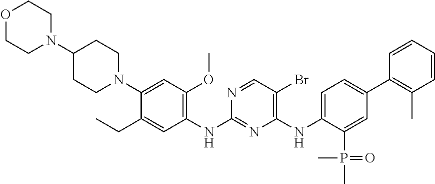 [SMILES: CCc1cc(Nc2ncc(Br)c(Nc3ccc(-c4ccccc4C)cc3P(C)(C)=O)n2)c(OC)cc1N1CCC(N2CCOCC2)CC1]

### Step 1: Synthesis of Compound K-078

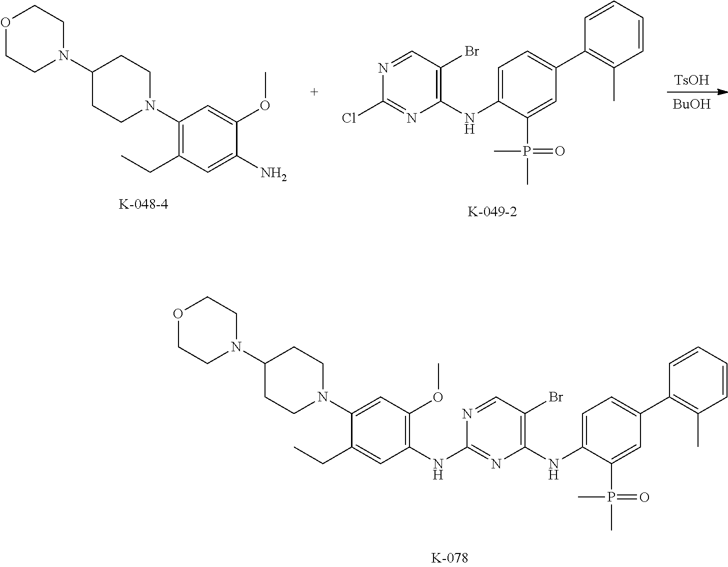

The compound K-078 was synthesized as the method described for compound K-018 using compound K-049-2 instead of raw compound K-018-5, compound K-093-4 instead of raw compound K-018-8. For compound K-078, MS: 733.26 [M+H]⁺

### Example 79: Synthesis of Compound K-079

### (2-((5-Bromo-2-((4-(4-(dimethylamino)piperidin-1-yl)-5-ethyl-2-methoxyphenyl)amino)pyrimidine-4-yl)amino)-5-(1-methyl-1H-pyrazolo[3,4-b]pyridin-3-yl)phenyl)dimethylphosphine oxide

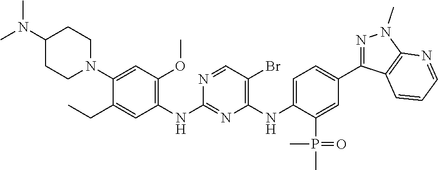 [SMILES: CCc1cc(Nc2ncc(Br)c(Nc3ccc(-c4nn(C)c5ncccc45)cc3P(C)(C)=O)n2)c(OC)cc1N1CCC(N(C)C)CC1]

### Step 1: Synthesis of Compound K-079-1

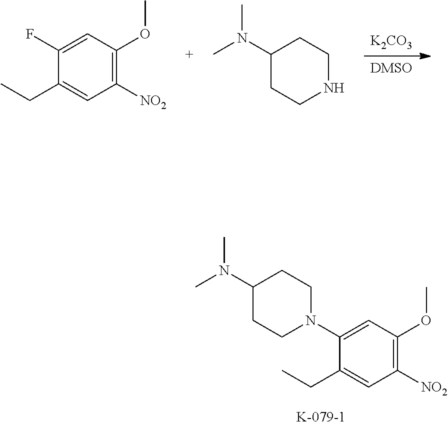

The compound K-079-1 was synthesized as the method described for compound K-024-1 using N,N-dimethylpiperidin-4-amine and 1-Ethyl-2-fluoro-4-methoxy-5-nitrobenzene instead of two raw compounds. For compound K-079-1, MS: 308.19 [M+H]⁺

### Step 2: Synthesis of Compound K-079-2

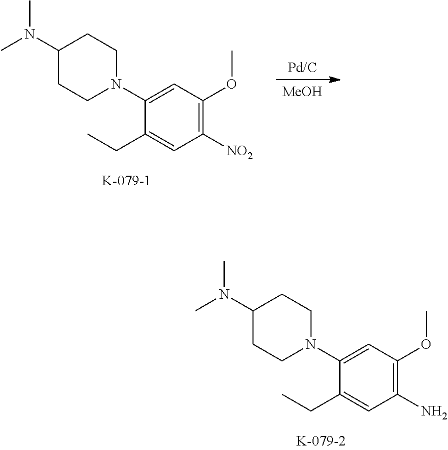

The compound K-079-2 was synthesized as the method described for compound K-051-6 using compound K-079-1 instead of raw compound K-051-5. For compound K-079-2, MS: 278.22 [M+H]⁺

### Step 3: Synthesis of Compound K-079

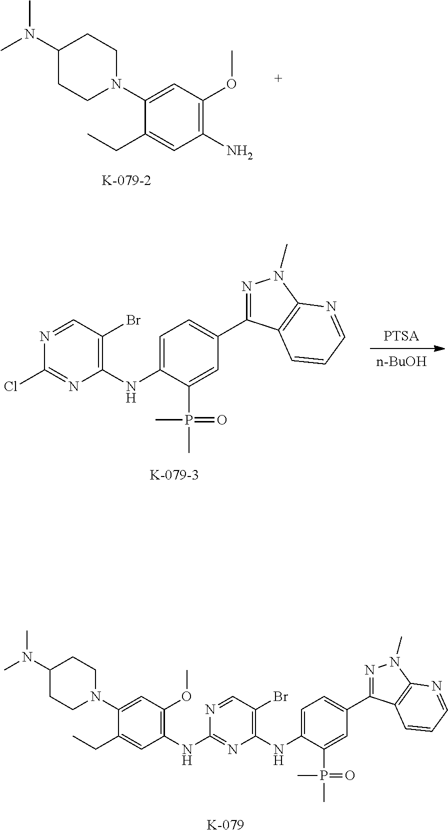

The compound K-079 was synthesized as the method described for compound K-019 using compound K-079-2 instead of raw compound K-019-3, compound K-079-2 instead of raw compound K-018-9. For compound K-079, MS: 732.25 [M+H]⁺

### Example 80: Synthesis of Compound K-080

### (2-((5-Bromo-2-((5-(1-ethyl-1H-pyrazol-4-yl)-2-methoxy-4-(4-(4-methylpiperazin-1-yl)piperidin-1-yl)phenyl)amino)pyrimidin-4-yl)amino)-5-(1,5-dimethyl-1H-pyrazol-3-yl)phenyl)dimethylphosphine oxide

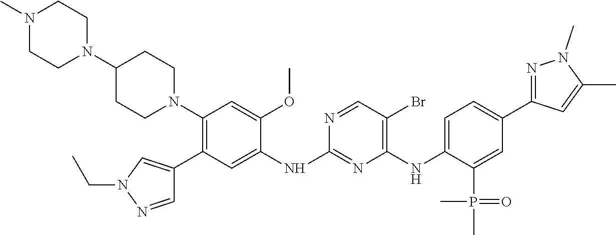

### Step 1: Synthesis of Compound K-080

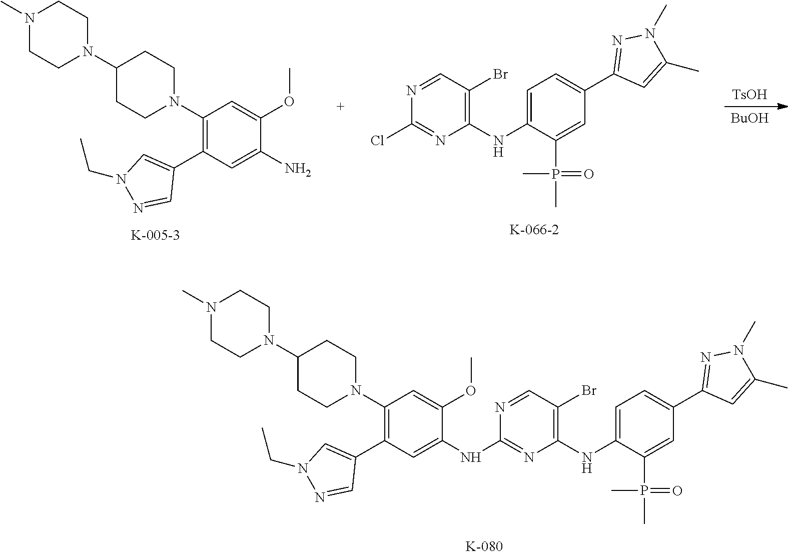

The compound K-080 was synthesized as the method described for compound K-018 using compound K-066-2 instead of raw compound K-018-5, compound K-005-3 instead of raw compound K-018-8. For compound K-080, MS: 816.31 [M+H]⁺

### Example 81: Synthesis of Compound K-081

### (2-((5-Bromo-2-((5-((4-(4-cyclopentylpiperazin-1-yl)-2-methoxy-5-(1-methyl-1H-pyrazol-4-ylphenyl)amino)pyrimidin)-4-yl)amino)-5-(4-methoxy-1H-pyrazol-1-yl)phenyl)dimethylphosphine oxide

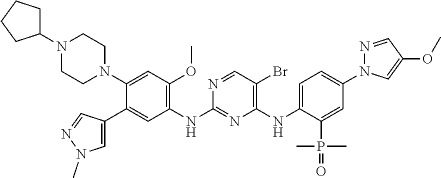 [SMILES: COc1cnn(-c2ccc(Nc3nc(Nc4cc(-c5cnn(C)c5)c(N5CCN(C6CCCC6)CC5)cc4OC)ncc3Br)c(P(C)(C)=O)c2)c1]

### Step 1: Synthesis of Compound K-081-2

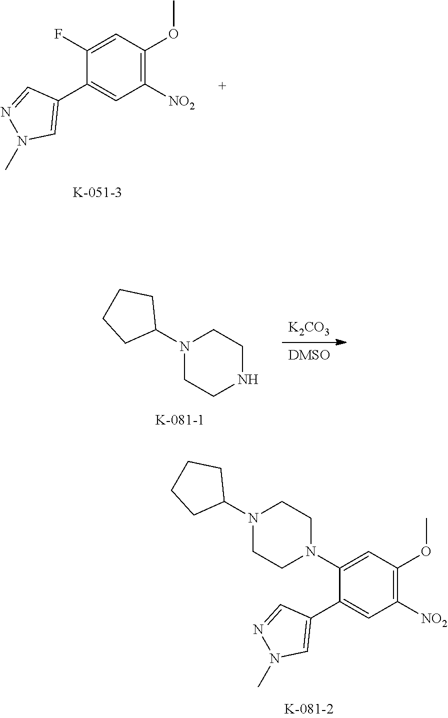

The compound K-081-2 was synthesized as the method described for compound K-051-5 using compound K-081-1 instead of raw compound K-051-4. For compound K-081-2, MS: 386.21 [M+H]⁺

### Step 2: Synthesis of Compound K-081-3

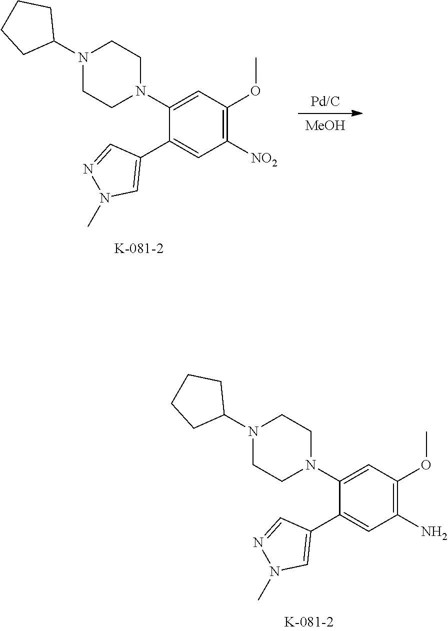 [SMILES: "MeOH" ; COc1cc(N2CCN(C3CCCC3)CC2)c(-c2cnn(C)c2)cc1[N+](=O)[O-] ; "Pd/C" ; COc1cc(N2CCN(C3CCCC3)CC2)c(-c2cnn(C)c2)cc1N]

The compound K-081-3 was synthesized as the method described for compound K-051-6 using compound K-081-2 instead of raw compound K-051-5. For compound K-081-3, MS: 356.24 [M+H]⁺

### Step 3: Synthesis of Compound K-081

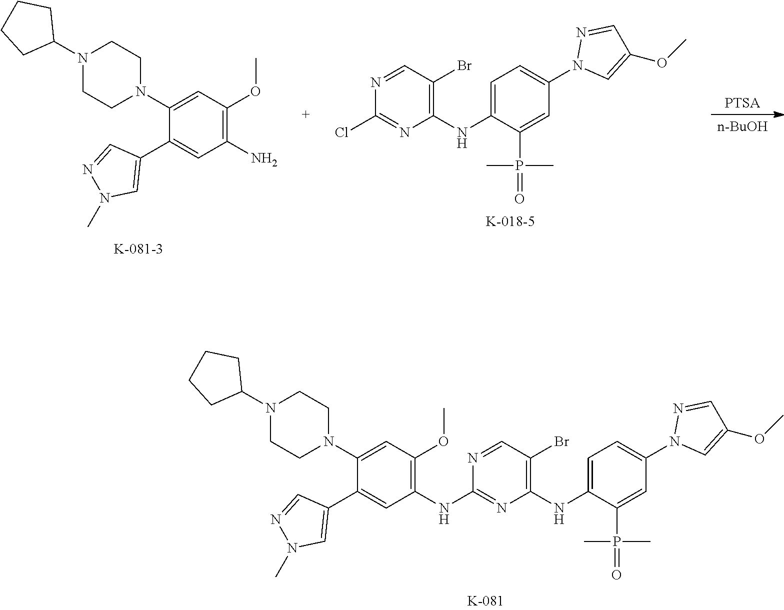

The compound K-081 was synthesized as the method described for compound K-019 using compound K-081-3 instead of raw compound K-019-8. For compound K-081, MS: 775.25 [M+H]⁺

### Example 82: Synthesis of Compound K-082

### 1-(4-(4-((5-Bromo-4-((2-(dimethylphosphate)-4-(4-methoxy-1H-pyrazol-1-yl)phenyl)amino)pyrimidin)-2-yl)amino)-5-methoxy-2-(1-methyl-1H-pyrazol-4-yl)phenyl)piperazin-1-yl-acetylacetone

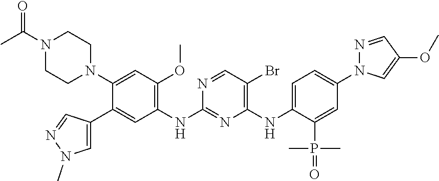 [SMILES: COc1cnn(-c2ccc(Nc3nc(Nc4cc(-c5cnn(C)c5)c(N5CCN(C(C)=O)CC5)cc4OC)ncc3Br)c(P(C)(C)=O)c2)c1]

### Step 1: Synthesis of compound K-082-2

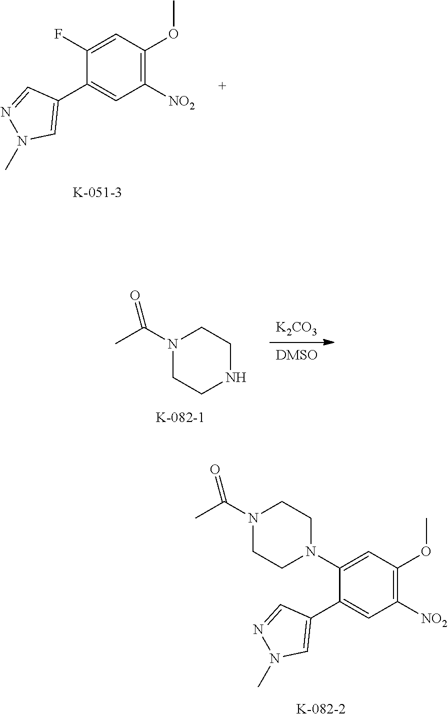

The compound K-082-2 was synthesized as the method described for compound K-051-5 using compound K-082-1 instead of raw compound K-051-4. For compound K-082-2. MS: 360.15 [M+H]⁺

### Step 2: Synthesis of Compound K-082-3

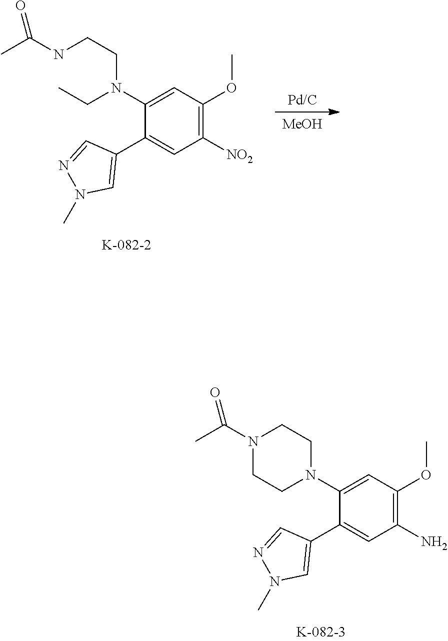 [SMILES: "MeOH" ; CCN(CCNC(C)=O)c1cc(OC)c([N+](=O)[O-])cc1-c1cnn(C)c1 ; "Pd/C" ; COc1cc(N2CCN(C(C)=O)CC2)c(-c2cnn(C)c2)cc1N]

The compound K-082-3 was synthesized as the method described for compound K-051-6 using compound K-082-2 instead of raw compound K-051-5. For compound K-082-3, MS: 330.19 [M+H]⁺

### Step 3: Synthesis of Compound K-082

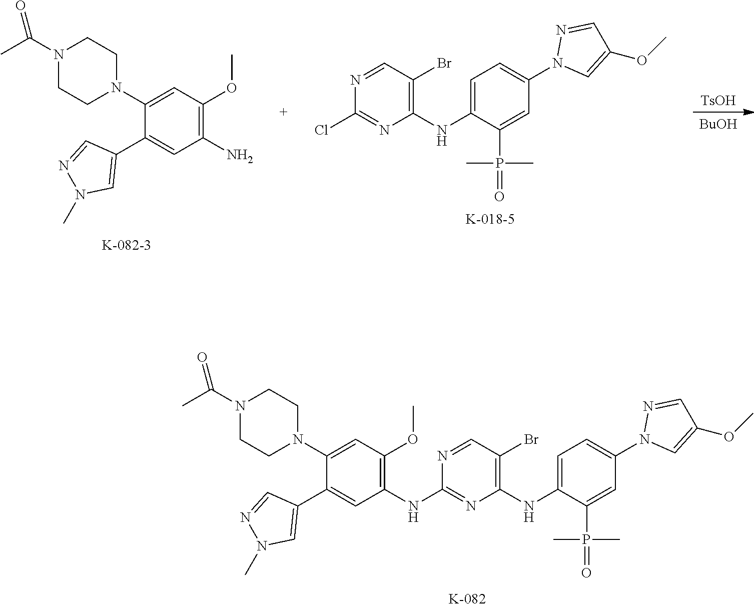

The compound K-082 was synthesized as the method described for compound K-018 using compound K-082-3 instead of raw compound K-018-8. For compound K-082, MS: 749.20 [M+H]⁺

### Example 83: Synthesis of Compound K-083

### (2-((5-Bromo-2-((2-methoxy-5-(1-methyl-1H-pyrazol-4-yl)-4-(4-(pyridin-3-yl)Piperazin-1-yl)phenyl)amino)pyrimidin-4-yl)amino)-5-(4-methoxy-1H-imidazol-1-yl)phenyl)dimethylphosphine oxide

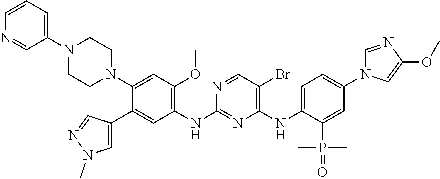 [SMILES: COc1cn(-c2ccc(Nc3nc(Nc4cc(-c5cnn(C)c5)c(N5CCN(c6cccnc6)CC5)cc4OC)ncc3Br)c(P(C)(C)=O)c2)cn1]

### Step 1: Synthesis of Compound K-083-2

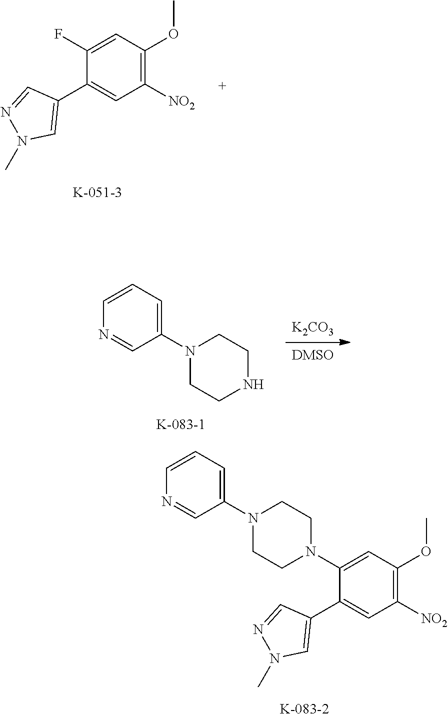

The compound K-083-2 was synthesized as the method described for compound K-051-5 using compound K-083-1 instead of raw compound K-051-4. For compound K-083-2, MS: 395.18 [M+H]⁺

### Step 2: Synthesis of Compound K-083-3

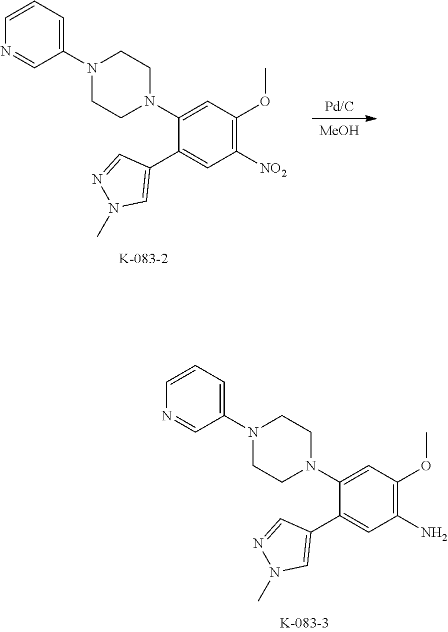 [SMILES: "MeOH" ; COc1cc(N2CCN(c3cccnc3)CC2)c(-c2cnn(C)c2)cc1[N+](=O)[O-] ; "Pd/C" ; COc1cc(N2CCN(c3cccnc3)CC2)c(-c2cnn(C)c2)cc1N]

The compound K-083-3 was synthesized as the method described for compound K-051-6 using compound K-083-2 instead of raw compound K-051-5. For compound K-083-3, MS: 365.20 [M+H]⁺

### Step 3: Synthesis of Compound K-083

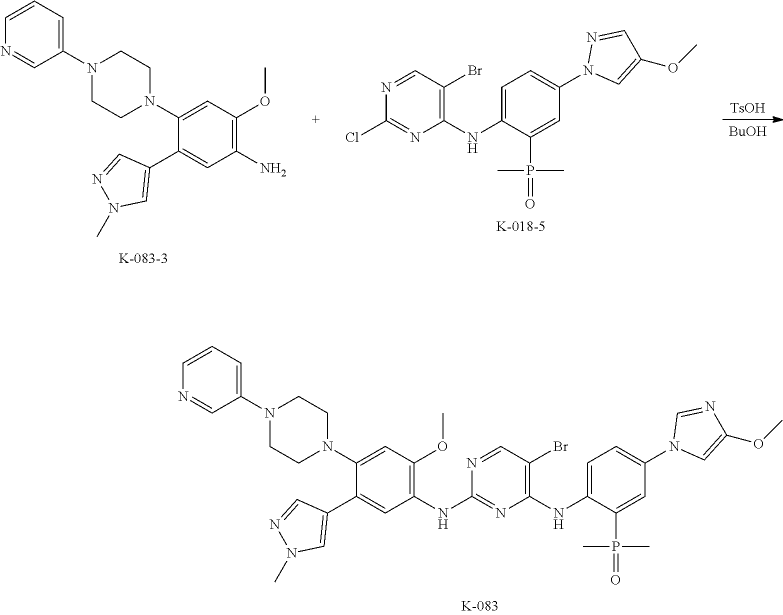

The compound K-083 was synthesized as the method described for compound K-018 using compound K-083-3 instead of raw compound K-018-8. For compound K-083, MS: 784.22 [M+H]⁺

### Example 84: Synthesis of Compound K-084

### (2-((5-Brno-2-((5-methoxy-2-methyl-4-(4-(4-methylpiperazin-1-yl)piperidin-1-yl)phenyl]amino]amino]pyrimidin-4-yl)amino)-5-(4-(trifluoromethyl)-1H-pyrazol-1-yl)phenyl)dimethylphosphine oxide

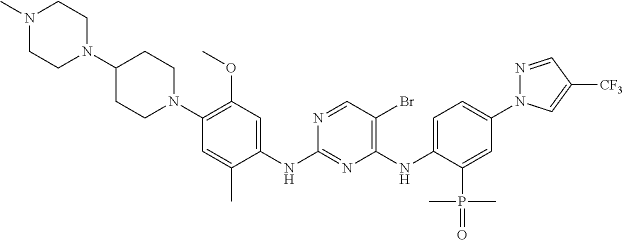 [SMILES: COc1cc(Nc2ncc(Br)c(Nc3ccc(-n4cc(C(F)(F)F)cn4)cc3P(C)(C)=O)n2)c(C)cc1N1CCC(N2CCN(C)CC2)CC1]

### Step 1: Synthesis of Compound K-084-2

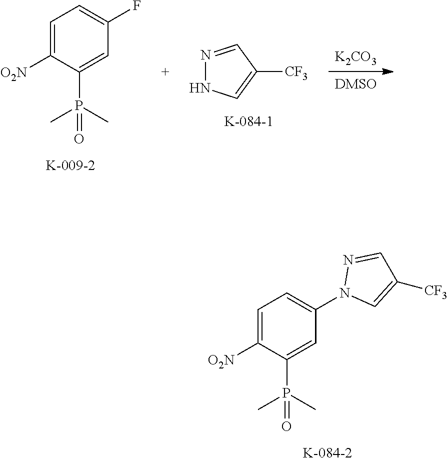

The compound K-084-2 was synthesized as the method described for compound K-009-3 using compound K-084-1 instead of raw compound K-009-2A. For compound K-184-2, MS: 334.05 [M+H]⁺

### Step 2: Synthesis of Compound K-084-3

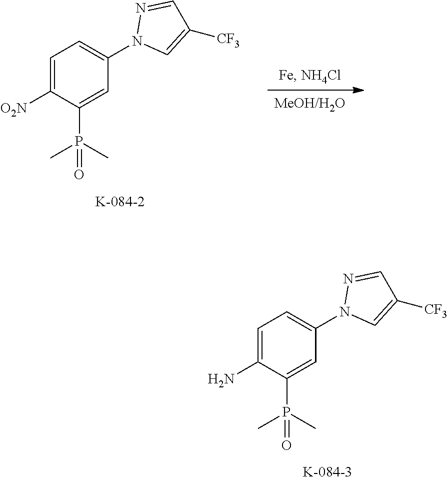

The compound K-084-3 was synthesized as the method described for compound K-009-4 using compound K-084-2 instead of raw compound K-009-3. For compound K-084-3, MS: 304.07 [M+H]⁺

### Step 3: Synthesis of Compound K-084-4

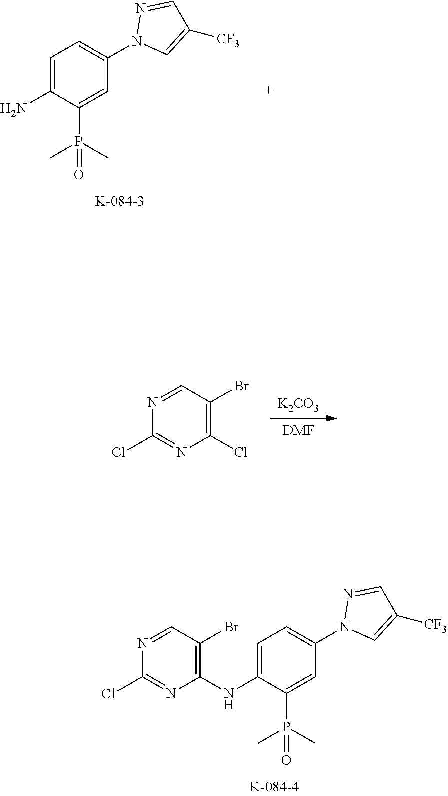

The compound K-084-4 was synthesized as the method described for compound K-009-5 using compound K-084-3 instead of raw compound K-009-4. For compound K-084-4, MS: 493.97 [M+H]⁺

### Step 4: Synthesis of Compound K-084

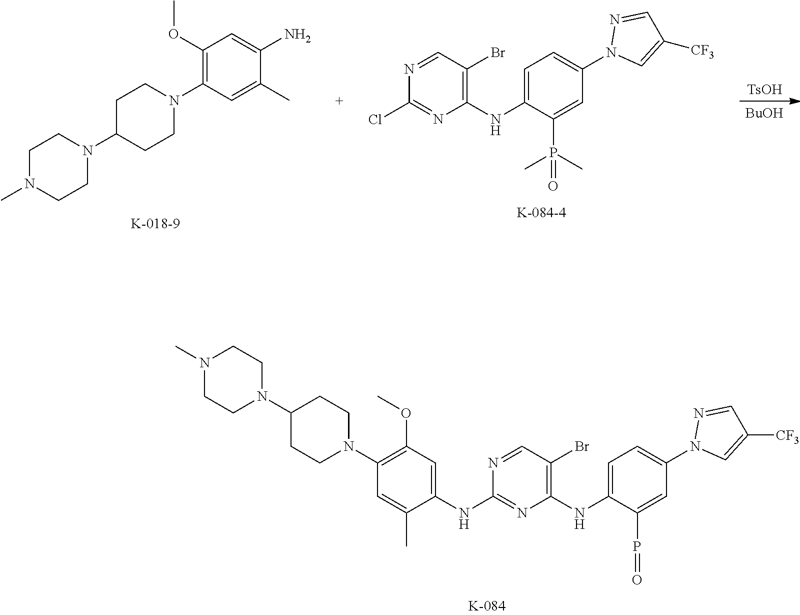

The compound K-084 was synthesized as the method described for compound K-009 using compound K-084-4 instead of raw compound K-009-5, compound K-018-9 instead of raw compound K-009-9. For compound K-084, MS: 776.23 [M+H]⁺

### Example 85: Synthesis of Compound K-085

### (2-((5-Bromo-2-((2-methoxy-5-(1-methyl-1H-pyrazol-4-yl)-4-(4-(4-methylpiperazin-1-yl)piperidin-1-)phenyl)amino)pyrimidin-4-yl)amino)-5-(4-(trifluoromethyl)-1H-pyrazol-1-yl)phenyl)dimethylphosphine oxide

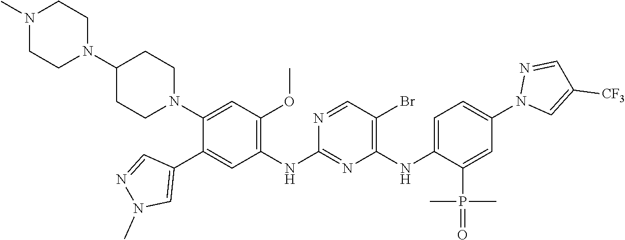 [SMILES: COc1cc(N2CCC(N3CCN(C)CC3)CC2)c(-c2cnn(C)c2)cc1Nc1ncc(Br)c(Nc2ccc(-n3cc(C(F)(F)F)cn3)cc2P(C)(C)=O)n1]

### Step 1: Synthesis of Compound K-085

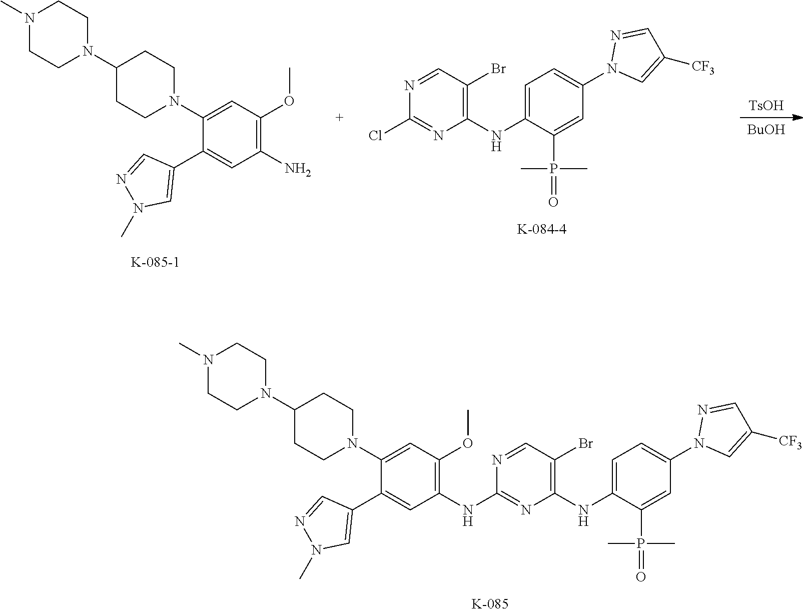

The compound K-085 was synthesized as the method described for compound K-009 using compound K-084-4 instead of raw compound K-009-5, compound K-085-1 instead of raw compound K-009-9. For compound K-085, MS: 842.26 [M+H]⁺

### Example 86: Synthesis of Compound K-086

### (2-((5-Bromo-2-((2-methoxy-5-methyl-4-(4-(4-methylpiperazin-1-yl)piperidin-1-yl)phenyl))amino]amino]pyrimidin-4-yl)amino)-5-(4-(trifluoromethyl)-1H-pyrazol-1-yl)phenyl)dimethylphosphine oxide

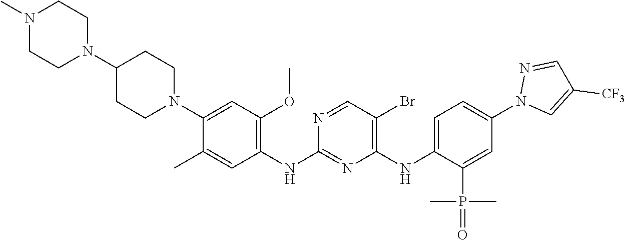 [SMILES: COc1cc(N2CCC(N3CCN(C)CC3)CC2)c(C)cc1Nc1ncc(Br)c(Nc2ccc(-n3cc(C(F)(F)F)cn3)cc2P(C)(C)=O)n1]

### Step 1: Synthesis of Compound K-086

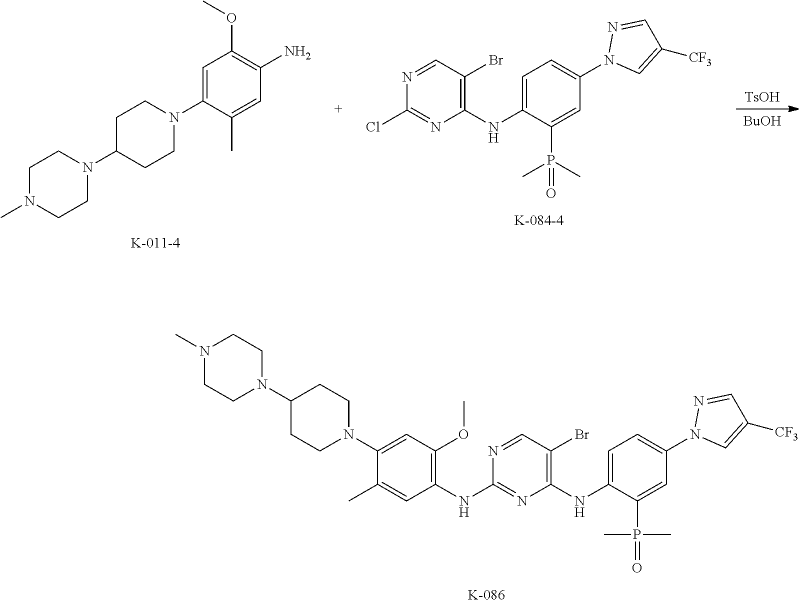

The compound K-086 was synthesized as the method described for compound K-009 using compound K-084-4 instead of raw compound K-009-5, compound K-011-4 instead of raw compound K-009-9. For compound K-086, MS: 776.23 [M+H]⁺

### Example 87: Synthesis of Compound K-087

### (2-((2-((4-([[1,4′-bispiperidin]-1-1-yl)-2-methoxy-5-methylphenyl)amino)-5-bromopyrimidin-4-yl)amino)-5-(4-methoxy-1H-pyrazol-1-yl)phenyl)dimethylphosphine oxide

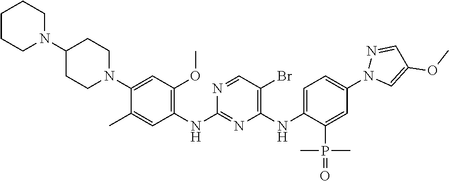 [SMILES: COc1cnn(-c2ccc(Nc3nc(Nc4cc(C)c(N5CCC(N6CCCCC6)CC5)cc4OC)ncc3Br)c(P(C)(C)=O)c2)c1]

### Step 1: Synthesis of Compound K-087-1

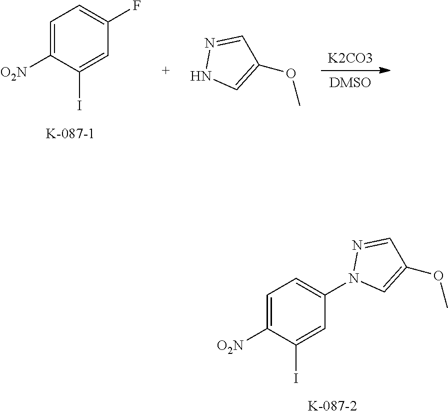

K-087-1 and 4-methoxy-1h-pyrazole were dissolved in DMF in a reactor equipped with a stir bar and nitrogen protector, and K₂CO₃ was added at room temperature; the gas in reactor was replaced with nitrogen for three times. After well stirring, the solution was transferred to an oil bath, heated to 100° C., and stirred at 100° C. for 16 hr. The reaction was complete as monitored by LCMS. The reaction solution was cooled down to room temperature, poured into water, and extracted with ethyl acetate (30 ml) for three times; the organic phase was concentrated by rotary evaporator. Silica gel was added to the concentrated organic phase to mix the sample, and then which was separated and purified by PE:EtOAc column with gradient from 0% to 80%. After that, it was concentrated and dried to obtain the compound K-087-2 (2.5 g), MS: 345.96 [M+H]⁺

### Step 2: Synthesis of Compound K-087-3

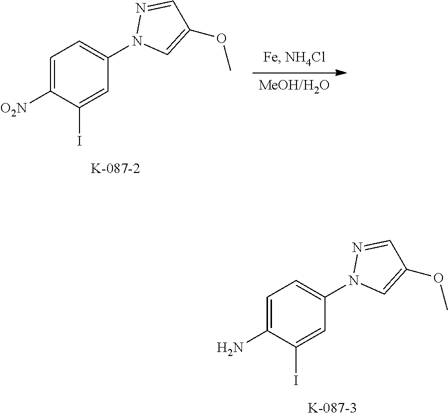

K-087-2 was dissolved in MeOH and H₂O in a reactor equipped with a stir bar and nitrogen protector, Fe powder and NH₄Cl were added at room temperature, and the gas in reactor was replaced with nitrogen for three times. After well stirring, the solution was transferred to an oil bath, heated to 80° C., and stirred at 80° C. for 3 hr. The reaction was complete as monitored by LCMS. The reaction solution was cooled down to room temperature, poured into water (50 mL), and extracted with DCM/MeOH=10:1 (30 mL) for three times; the organic phase was dried over anhydrous sodium sulfate; after filtration of anhydrous sodium sulfate, the organic phase was concentrated by rotary evaporator. Silica gel was added to the concentrated organic phase to mix the sample, and then which was separated and purified by DCM/MeOH with gradient from 0% to 10%. After that, it was concentrated and dried to obtain the compound K-087-3 (1.8 g), MS: 315.99 [M+H]⁺

### Step 3: Synthesis of Compound K-087-4

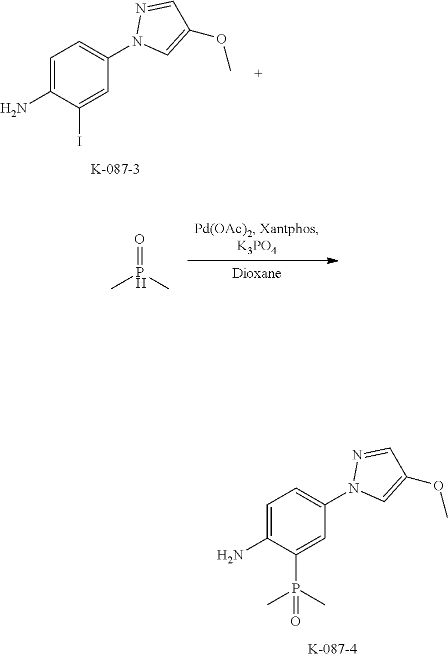

Add compound K-087-3, dimethylphosphine oxide, K₃PO₄, Pd(OAc)₂, Xantphos and dioxane successively to the reaction flask, heat to 120° C. under nitrogen protection, and stir with heating for 16 hr. The reaction was complete as monitored by LCMS, and then stopped. The reaction solution was poured into water (50 mL), and extracted with dichloromethane (3×50 mL); the organic phase was washed with saturated brine (3×30 mL), dried over anhydrous sodium sulfate, separated and purified by column chromatography (dichloromethane:methanol=15:1), then concentrated and dried to obtain yellow solid of desired product K-087-4, MS: 266.10 [M+H]⁺

### Step 4: Synthesis of Compound K-087-5

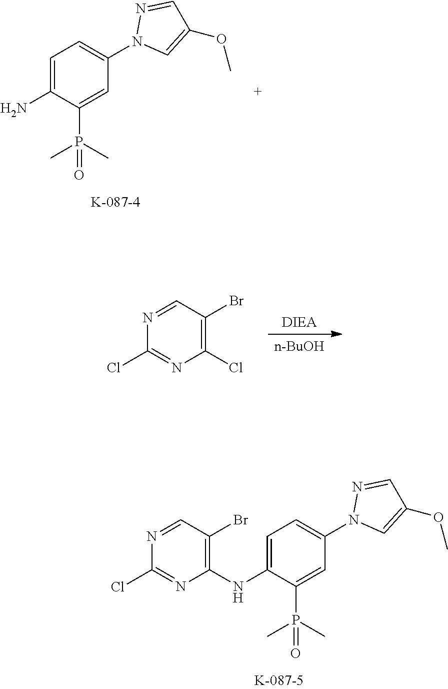

Add compound K-087-4, 5-Bromo-2,4-dichloropyrimidine, DIEA and n-butanol successively to the reaction flask, heat to 130° C., and stir with heating for 36 hr. The reaction was complete as monitored by LCMS, and then stopped. The reaction solution was poured into water (50 mL), and extracted with ethyl acetate (3×50 mL); the organic phase was washed with saturated brine (3×30 mL), dried over anhydrous sodium sulfate, separated and purified by column chromatography (dichloromethane:methanol=13:1), and the solvent was removed to obtain yellow solid of desired product compound K-087-5, MS: 456.99 [M+H]⁺

### Step 5: Synthesis of Compound K-087-8

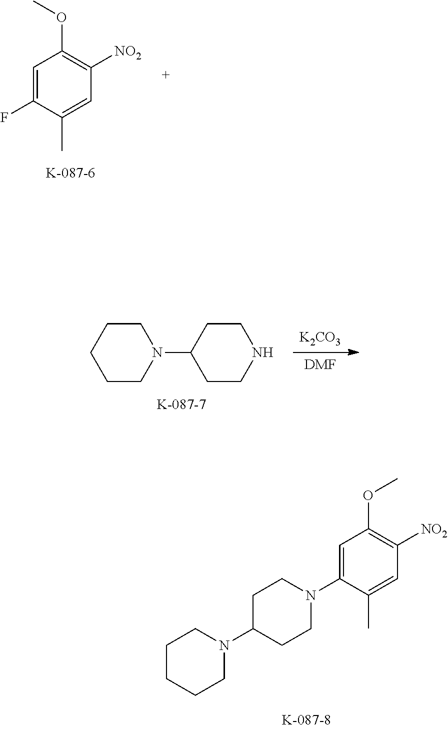

Add compounds K-087-6, K-087-7, K₂CO₃ and DMF successively to the reaction flask, heat to 90° C., and stir with heating for 12 hr. The reaction was complete as monitored by LCMS, and then stopped. The reaction solution was poured into water (100 mL), filtered with suction, and the filter cake was washed with water and dried to obtain the yellow solid of desired product compound K-087-8, MS: 334.21 [M+H]⁺

### Step 6: Synthesis of Compound K-087-9

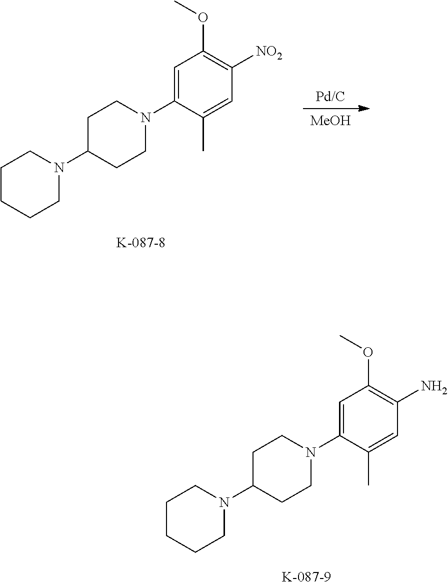 [SMILES: "MeOH" ; COc1cc(N2CCC(N3CCCCC3)CC2)c(C)cc1[N+](=O)[O-] ; "Pd/C" ; COc1cc(N2CCC(N3CCCCC3)CC2)c(C)cc1N]

Add compound K-087-8, Pd/C (10%) and MeOH successively to the reaction flask, introduce H₂; the reaction solution was stirred at room temperature for 3 hr. The reaction was complete as monitored by LCMS, and then stopped. The solution was filtered with suction, and rinsed with methanol (20 mL); collect the organic phase, remove the solvent, and obtain the reddish-brown liquid of desired product compound K-087-9, MS: 304.23 [M+H]⁺

### Step 7: Synthesis of Compound K-087

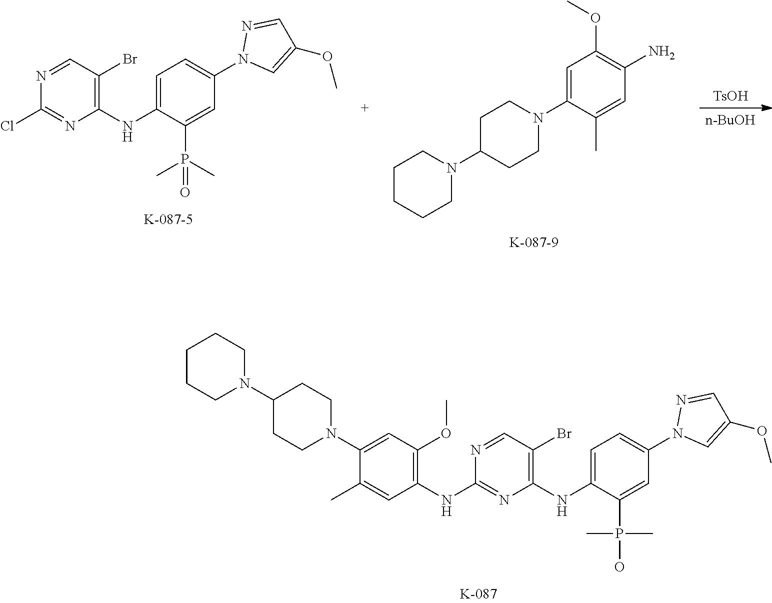

Add compounds K-087-5, K-087-9, p-toluenesulfonic acid and n-butanol successively into the reaction flask, heat to 120° C., and stir for 12 hr. The reaction was complete as monitored by LCMS, and then stopped. The reaction solution was poured into sodium carbonate solution (5 mL), and extracted with dichloromethane (3×50 mL); the organic phase was washed with saturated brine (3×30 mL), dried over anhydrous sodium sulfate, separated and purified by column chromatography (dichloromethane:methanol=12:1), and then concentrated to obtain an off-white solid compound K-087, MS: 724.24 [M+H]⁺

### Example 88: Synthesis of Compound K-088

### (2-((5-Bromo-2-((2-methoxy-5-methyl-4-(4-(1-methylpiperidin-4-yl)piperazin-1-yl)phenyl)amino]amino]pyrimidin-4-yl)amino)-5-(4-methoxy-1H-pyrazol-1-yl)phenyl)dimethylphosphine oxide

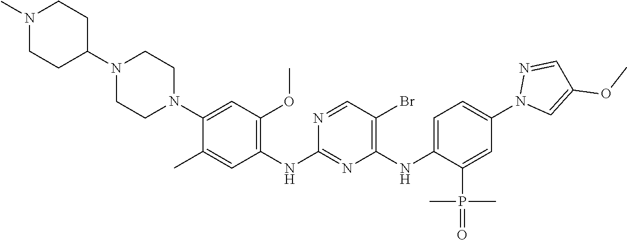

The compounds K-088-2, K-088-3, K-088-4 and K-088-5 were synthesized as the procedure described for compound K-087, except that the reaction raw materials were replaced. The raw materials used in this example were the intermediates or commercially available compounds synthesized in other examples of the invention.

### Step 1: Synthesis of Compound K-088-8

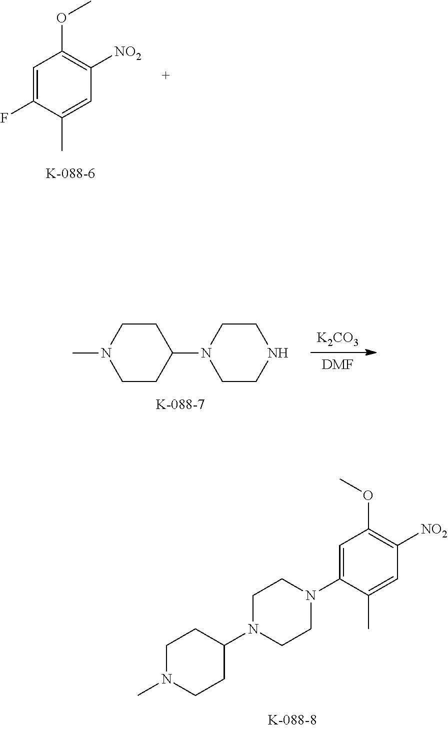

Add compounds K-088-6, K-088-7, K₂CO₃ and DMF successively to the reaction flask, heat to 90° C., and stir with heating for 12 hr. The reaction was complete as monitored by LCMS, and then stopped. The reaction solution was poured into water (100 mL), filtered with suction, and the filter cake was washed with water and dried to obtain the yellow solid of desired product compound K-088-8, MS: 349.22 [M+H]⁺

### Step 6: Synthesis of Compound K-088-9

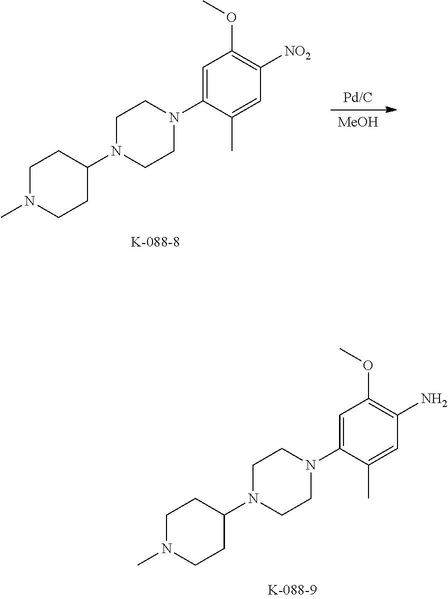 [SMILES: "MeOH" ; COc1cc(N2CCN(C3CCN(C)CC3)CC2)c(C)cc1[N+](=O)[O-] ; "Pd/C" ; COc1cc(N2CCN(C3CCN(C)CC3)CC2)c(C)cc1N]

Add compound K-088-8, Pd/C (10%) and MeOH successively to the reaction flask, introduce H₂; the reaction solution was stirred at room temperature for 3 hr. The reaction was complete as monitored by LCMS, and then stopped. The solution was filtered with suction, and rinsed with methanol (20 mL); collect the organic phase, remove the solvent, and obtain the reddish-brown liquid of desired product compound K-088-9, MS: 319.25 [M+H]⁺

### Step 7: Synthesis of Compound K-088

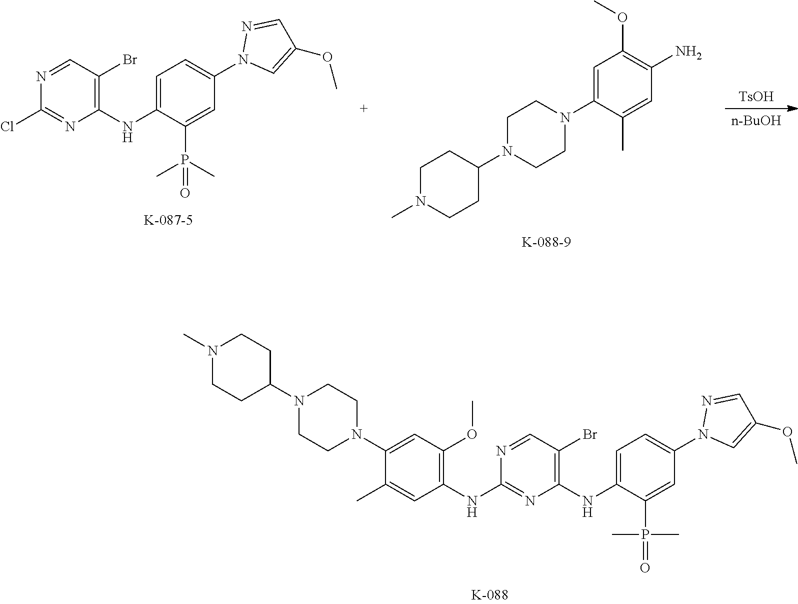

Add compounds K-087-5, K-088-9, p-toluenesulfonic acid and n-butano successively into the reaction flask, heat to 120° C., and stir for 12 hr. The reaction was complete as monitored by LCMS, and then stopped. The reaction solution was poured into sodium carbonate solution (5 mL), and extracted with dichloromethane (3×50 mL); the organic phase was washed with saturated brine (3×30 mL), dried over anhydrous sodium sulfate, separated and purified by column chromatography (dichloromethane:methanol=12:1), and then concentrated to obtain off-white solid of compound K-088, MS: 739.25 [M+H]⁺

### Example 89: Synthesis of Compound K-089

### (2-((5-Bromo-2-((4-(4-cyclopentylpiperazin-1-yl)-2-methoxy-5-methylphenyl)amino)pyrimidin-4-yl)amino)-5-(4-methoxy-1H-pyrazol-1-yl)phenyl)dimethylphosphine oxide

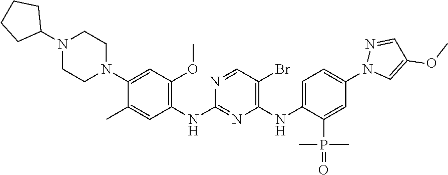 [SMILES: COc1cnn(-c2ccc(Nc3nc(Nc4cc(C)c(N5CCN(C6CCCC6)CC5)cc4OC)ncc3Br)c(P(C)(C)=O)c2)c1]

Compounds K-089-2, K-089-3, K-089-4 and K-089-5 were synthesized as the procedure described for compound K-087, except that the reaction raw materials were replaced. The raw materials used in this example were the intermediates or commercially available compounds synthesized in other examples of the invention

### Step 1: Synthesis of Compound K-089-8

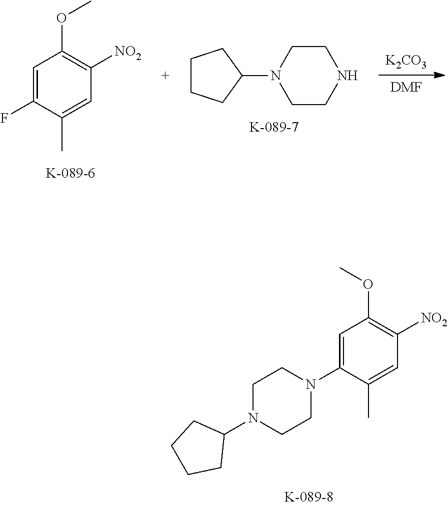

Add compounds K-089-6, K-089-7, K₂CO₃ and DMF to the reaction flask, heat to 90° C., and stir with heating for 12 hr. The reaction was complete as monitored by LCMS, and then stopped. The reaction solution was poured into water (100 mL), filtered with suction, and the filter cake was washed with water and dried to obtain yellow solid of desired product K-089-8, MS: 320.19 [M+H]⁺

### Step 6: Synthesis of Compound K-089-9

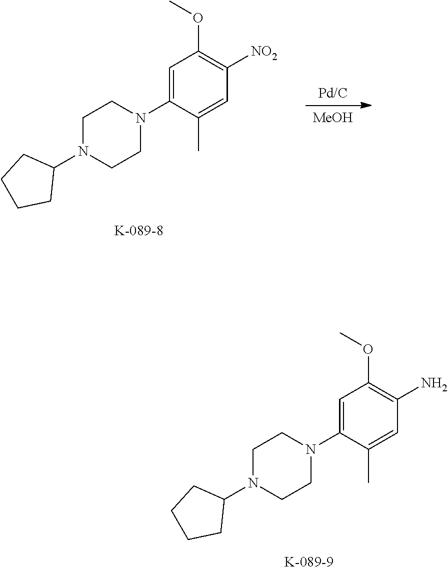 [SMILES: "MeOH" ; COc1cc(N2CCN(C3CCCC3)CC2)c(C)cc1[N+](=O)[O-] ; "Pd/C" ; COc1cc(N2CCN(C3CCCC3)CC2)c(C)cc1N]

Add compound K-089-8, Pd/C (10%) and MeOH successively to the reaction flask, introduce H₂; the reaction solution was stirred at room temperature for 3 hr. The reaction was complete as monitored by LCMS, and then stopped. The solution was filtered with suction, and rinsed with methanol (20 mL); collect the organic phase, remove the solvent, and obtain the reddish-brown liquid of desired product compound K-089-9, MS: 320.19 [M+H]⁺

### Step 7: Synthesis of Compound K-089

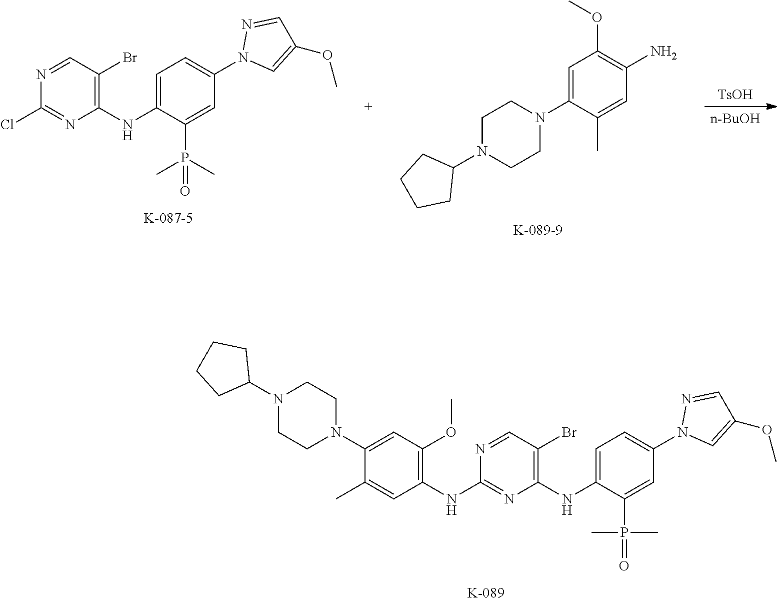

Add compound K-087-5, K-089-9, p-toluenesulfonic acid and n-butanol successively into the reaction flask, heat to 120° C., and stir for 12 hr. The reaction was complete as monitored by LCMS, and then stopped. The reaction solution was poured into sodium carbonate solution (5 mL), and extracted with dichloromethane (3×50 mL); the organic phase was washed with saturated brine (3×30 mL), dried over anhydrous sodium sulfate, separated and purified by column chromatography (dichloromethane:methanol=12:1), and then concentrated to obtain off-white solid of compound K-089, MS: 710.23 [M+H]⁺

### Example 90: Synthesis of Compound K-090

### (2-((5-Bromo-2-((5-ethyl-2-methoxy-4-(4-(4-methylpiperazin-1-yl)piperidin-1-yl)phenyl)amino)amino]pyrimidin-4-yl)amino)-5-(4-(methoxymethyl)-1H-pyrazol-1-yl)phenyl)dimethylphosphine oxide

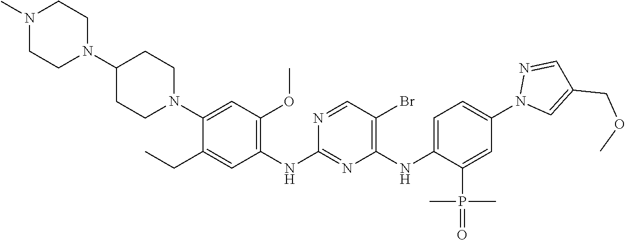 [SMILES: CCc1cc(Nc2ncc(Br)c(Nc3ccc(-n4cc(COC)cn4)cc3P(C)(C)=O)n2)c(OC)cc1N1CCC(N2CCN(C)CC2)CC1]

### Step 1: Synthesis of Compound K-090-2

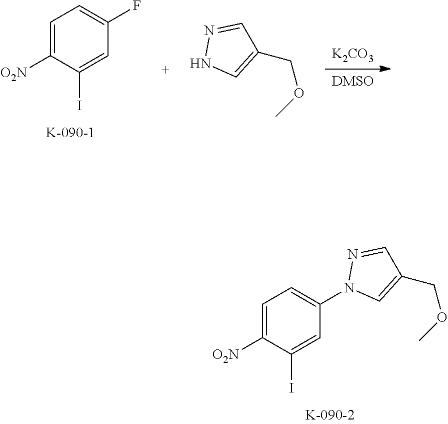

K-090-1 and 4-(Methoxy methyl)-1H-pyrazole were dissolved in DMF in a reactor equipped with a stir bar and nitrogen protector, K₂CO₃ was added to the solution at room temperature, and the gas in reactor was replaced with nitrogen for three times. After well stirring, the solution was transferred to an oil bath, heated to 100° C., and stirred at 100° C. for 16 hr. The reaction was complete as monitored by LCMS. The reaction solution was cooled down to room temperature, poured into water, and extracted with ethyl acetate (30 mL) for three times; the organic phase was concentrated by rotary evaporator. Silica gel was added to the concentrated organic phase to mix the sample, and then which was separated and purified by PE:EtOAc column with gradient from 0% to 80%. After that, it was concentrated and dried to obtain the compound K-090-2 (2.5 g), MS: 359.98 [M+H]⁺

### Step 2: Synthesis of Compound K-090-3

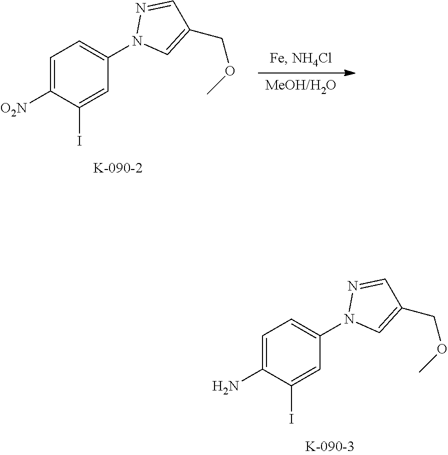

K-090-2 was dissolved in MeOH and H₂O in a reactor equipped with a stir bar and nitrogen protector, Fe powder and NH₄Cl were added at room temperature, and the gas in reactor was replaced with nitrogen for three times. After well stirring, the solution was transferred to an oil bath, heated to 80° C., and stirred at 80° C. for 3 hr. The reaction was complete as monitored by LCMS. The reaction solution was cooled down to room temperature, poured into water, and extracted with DCM/MeOH-10:1 (30 ml) for three times; the organic phase was dried over anhydrous sodium sulfate; after filtration of anhydrous sodium sulfate, the organic phase was concentrated by rotary evaporator. Silica gel was added to the concentrated organic phase to mix the sample, and then which was separated and purified by DCM/MeOH with gradient from 0% to 10%. After that, it was concentrated and dried to obtain the compound K-090-3, MS: 330.00 [M+H]⁺

### Step 3: Synthesis of Compound K-090-4

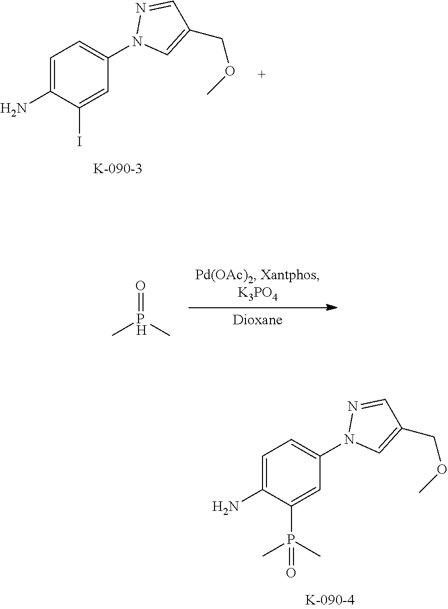

Add compound K-090-3, dimethylphosphine oxide. K₃PO₄, Pd(OAc)₂, Xantphos and dioxane to the reaction flask, heat to 120° C. under nitrogen protection, and stir with heating for 16 hr. The reaction was complete as monitored by LCMS, and then stopped. The reaction solution was poured into water (5 mL), and extracted with dichloromethane (3×50 mL); the organic phase was washed with saturated brine (3×30 mL), dried over anhydrous sodium sulfate, separated and purified by column chromatography (dichloromethane:methanol=15:1), then concentrated and dried to obtain yellow solid of desired product K-090-4, MS: 280.12 [M+H]⁺

### Step 4: Synthesis of Compound K-090-5

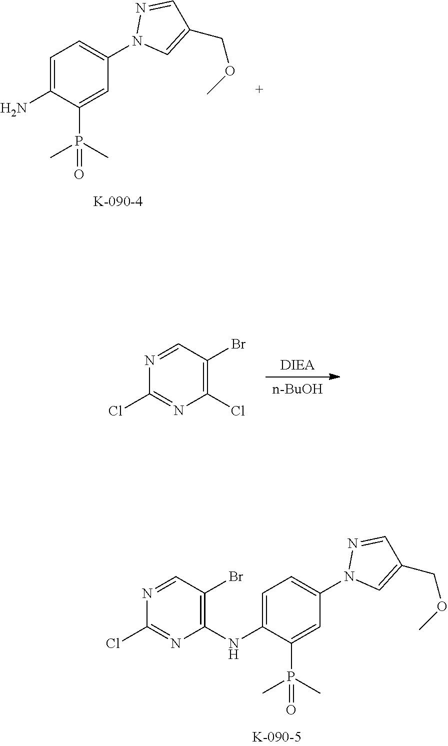

Add compound K-090-4, 5-Bromo-2,4-dichloropyrimidine, DIEA and n-butanol successively to the reaction flask, heat to 130° C., and stir with heating for 16 hr. The reaction was complete as monitored by LCMS, and then stopped. The reaction solution was poured into water (50 mL) solution, and extracted with ethyl acetate (3×50 mL); the organic phase was washed with saturated brine (3×30 mL), dried over anhydrous sodium sulfate, separated and purified by column chromatography (dichloromethane:methanol=13:1), and the solvent was removed to obtain the yellow solid of desired product compound K-090-5, MS: 471.00 [M+H]⁺

### Step 5: Synthesis of Compound K-090-8

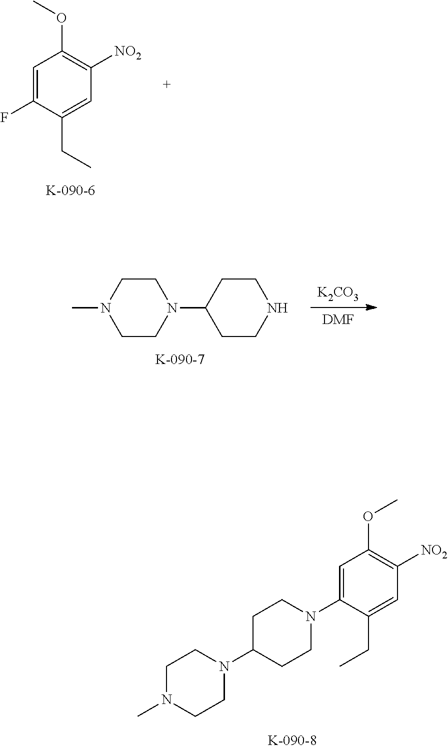

Add compounds K-090-6, K-090-7, K₂CO₃ and DMF to the reaction flask, heat to 90° C., and stir with heating for 12 hr. The reaction was complete as monitored by LCMS, and then stopped. The reaction solution was poured into water (100 mL), filtered with suction, and the filter cake was washed with water and dried to obtain yellow solid of desired product compound K-090-8, MS: 363.24 [M+H]⁺

### Step 6: Synthesis of Compound K-090-9

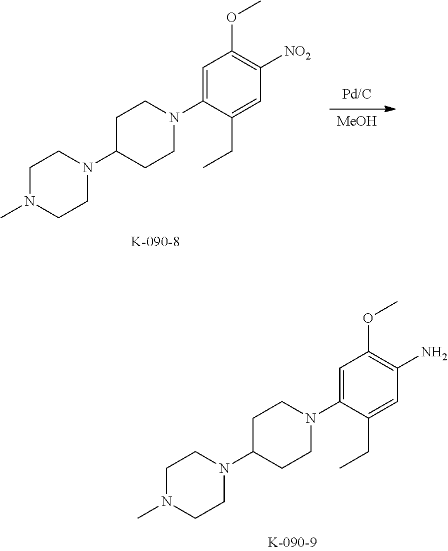 [SMILES: "MeOH" ; CCc1cc([N+](=O)[O-])c(OC)cc1N1CCC(N2CCN(C)CC2)CC1 ; "Pd/C" ; CCc1cc(N)c(OC)cc1N1CCC(N2CCN(C)CC2)CC1]

Add compound K-090-8, Pd/C (10%) and MeOH to the reaction flask, and introduce H₂; the reaction solution was stirred at room temperature for 3 hr. The reaction was complete as monitored by LCMS, and then stopped. The solution was filtered with suction, and rinsed with methanol (20 mL); collect the organic phase, remove the solvent, and obtain the reddish-brown liquid of desired product compound K-090-9, MS: 333.26 [M+H]⁺

### Step 7: Synthesis of Compound K-090

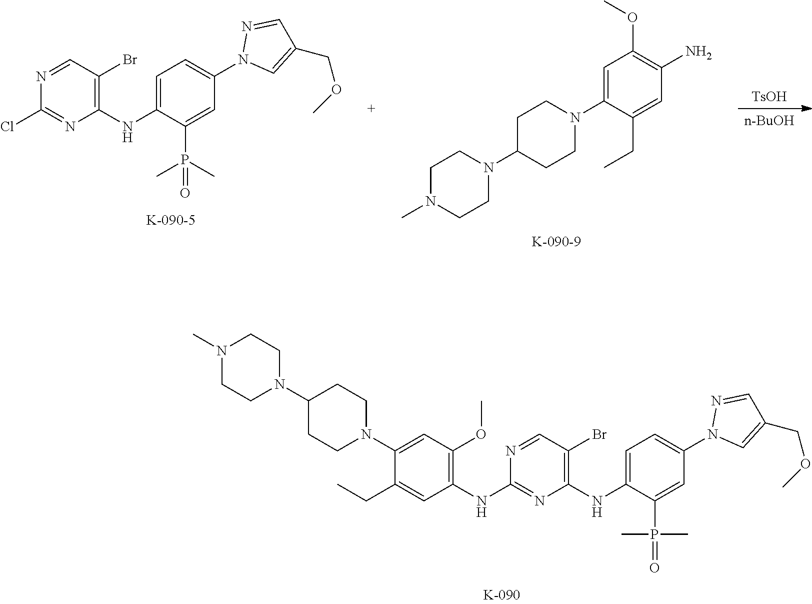

Add compound K-090-5, K-090-9, p-toluenesulfonic acid and n-butanol into the reaction flask, heat to 120° C., and stir for 12 hr. The reaction was complete as monitored by LCMS, and then stopped. The reaction solution was poured into sodium carbonate solution (5 mL), and extracted with dichloromethane (3×50 mL); the organic phase was washed with saturated brine (3×30 mL), dried over anhydrous sodium sulfate, separated and purified by column chromatography (dichloromethane:methanol=12:1), and then concentrated to obtain off-white solid of compound K-090, MS: 766.29 [M+H]⁺

### Example 91: Synthesis of Compound K-091

### (2-((5-Bromo-2-((5-(1-ethyl-1H-pyrazol-4-yl)-2-methoxy-4-(4-(4-methylpiperazin-1-yl)piperidin-1-yl)phenyl)amino)pyrimidin-4-yl)amino)-5-(1H-indol-7-yl)phenyl)dimethylphosphine oxide

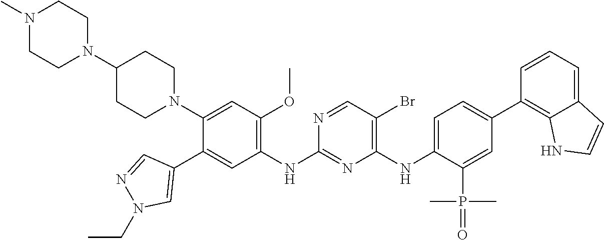 [SMILES: CCn1cc(-c2cc(Nc3ncc(Br)c(Nc4ccc(-c5cccc6cc[nH]c56)cc4P(C)(C)=O)n3)c(OC)cc2N2CCC(N3CCN(C)CC3)CC2)cn1]

### Step 3: Synthesis of Compound K-091-3

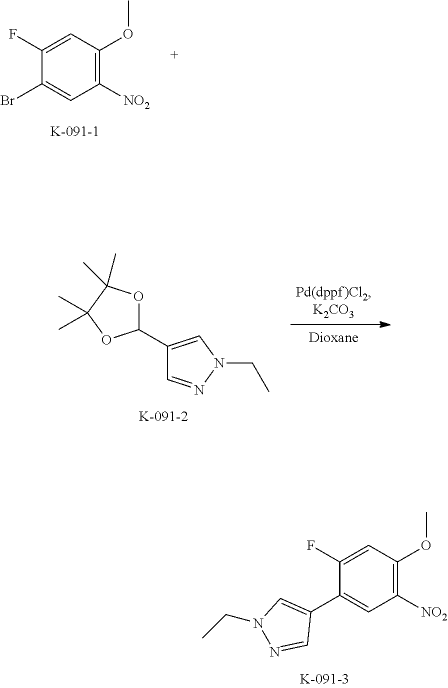 [SMILES: COc1cc(F)c(Br)cc1[N+](=O)[O-] ; CCn1cc(C2OC(C)(C)C(C)(C)O2)cn1 ; "Dioxane" ; "Pd(dppf)Cl2,K2CO3" ; CCn1cc(-c2cc([N+](=O)[O-])c(OC)cc2F)cn1]

Add compounds K-091-1, K-091-2, Pd(dppf)Cl₂, K₂CO₃, dioxane (30 mL) and water (3 mL) to the reaction flask, heat to 100° C. under nitrogen protection, and stir with heating for 1.5 hr. The reaction was complete as monitored by LCMS, and then stopped. The reaction solution was poured into water (50 mL), and extracted with dichloromethane (3×50 mL); the organic phase was washed with saturated brine (3×30 mL), dried over anhydrous sodium sulfate, separated and purified by PE:EtOAc (0%-50%), then concentrated and dried to obtain yellow solid of desired product K-091-3, MS: 266.09 [M+H]⁺

### Step 4: Synthesis of Compound K-091-5

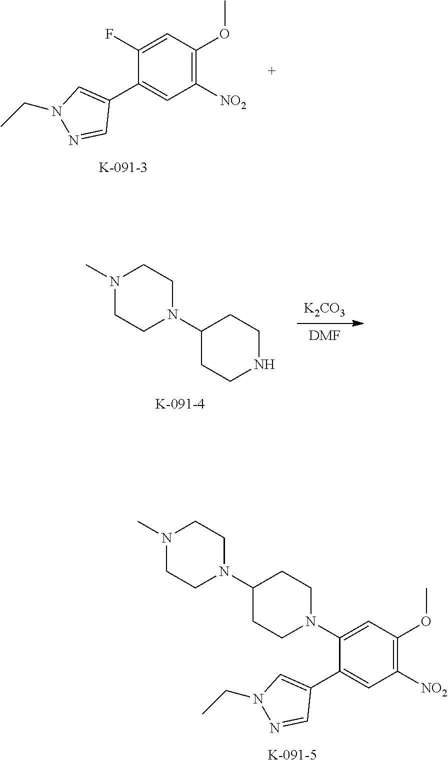

K-091-3 and K-091-4 were dissolved in DMF (20 mL) in a reactor equipped with a stir bar and nitrogen protector, K₂CO₃ was added to the solution at room temperature, and the gas in reactor was replaced with nitrogen for three times. After well stirring, the solution was transferred to an oil bath, heated to 100° C., and stirred at 90° C. for 16 hr. The reaction was complete as monitored by LCMS. The reaction solution was cooled down to room temperature, poured into water (50 mL), and extracted with ethyl acetate (30 mL) for three times; the organic phase was concentrated by rotary evaporator. Silica gel was added to the concentrated organic phase to mix the sample, and then which was separated and purified by PE:EtOAc column with gradient from 0% to 60%. After that, it was concentrated and dried to obtain the compound K-091-5 (400 mg), MS: 429.26 [M+H]⁺

### Step 5: Synthesis of Compound K-091-6

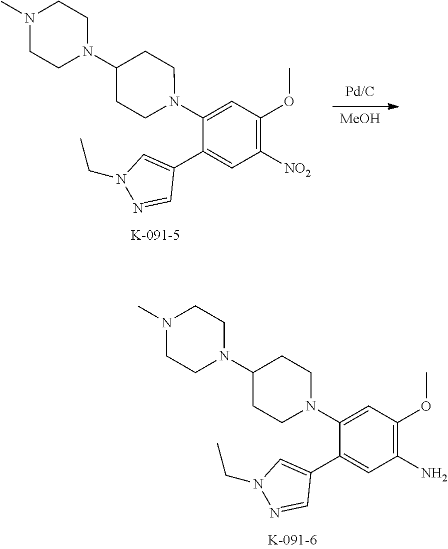

K-091-5 was dissolved in MeOH (20 mL) in a reactor equipped with a stir bar and nitrogen protector, and Pd/C was added to the solution at room temperature. After well stirring, the solution was stirred at 28° C. for 3 hr. The reaction was complete as monitored by LCMS. The reaction solution was filtered and concentrated. The crude product was directly put into the next step without purification. It was dried to obtain the compound K-091-6, MS: 399.28 [M+H]⁺

### Step 5: Synthesis of Compound K-091

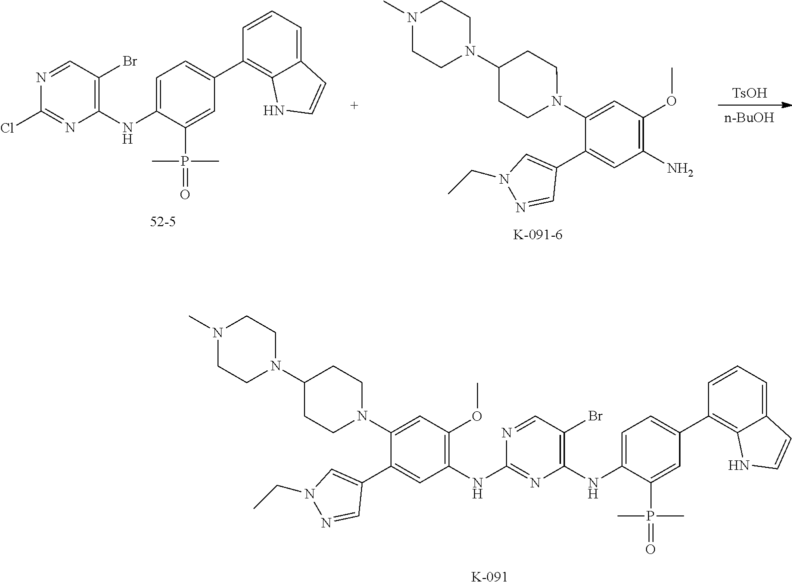

Add compound 52-5, K-091-6, p-toluenesulfonic acid and n-butanol successively to the reaction flask, heat to 120° C. and stir for 12 hr. The reaction was complete as monitored by LCMS, and then stopped. The reaction solution was poured into sodium carbonate solution (5 mL), and extracted with dichloromethane (3×50 mL); the organic phase was washed with saturated brine (3×30 mL), dried over anhydrous sodium sulfate, separated and purified by column chromatography (dichloromethane:methanol=12:1), and then concentrated to obtain off-white solid of compound K-091, MS: 838.30 [M+H]⁺

### Example 92: Synthesis of Compound K-092

### 3-(4-((5-Chloro-2-((5-ethyl-2-methoxy-4-(4-(4-methylpiperazin-1-yl)piperidin-1-yl)phenyl)amino)pyrimidin-4-yl)amino)-3-(dimethylphosphoryl)phenyl)pyridin-2(1H)-one

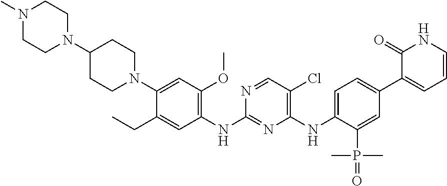 [SMILES: CCc1cc(Nc2ncc(Cl)c(Nc3ccc(-c4ccc[nH]c4=O)cc3P(C)(C)=O)n2)c(OC)cc1N1CCC(N2CCN(C)CC2)CC1]

### Step 1: Synthesis of Compound K-092

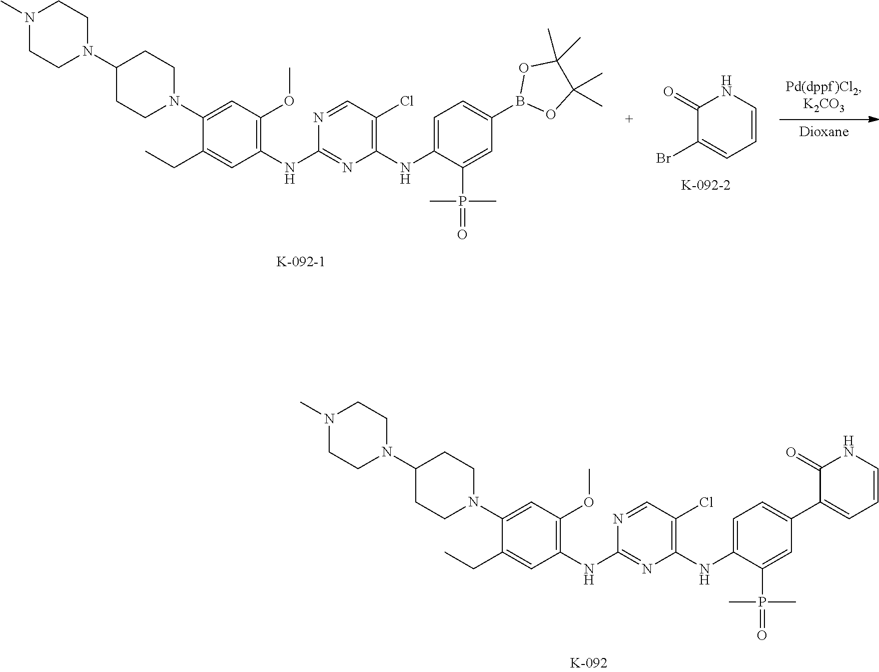

Ada compounds K-092-1, K-092-2, Pd(dppf)Cl₂, K₂CO₃, dioxane and water to the reaction flask, heat to 110° C. and stir with heating for 16 hr. The reaction was complete as monitored by LCMS, and then stopped. The reaction solution was poured into water (50 mL), and extracted with dichloromethane (3×50 mL); the organic phase was washed with saturated brine (3×30 mL), dried over anhydrous sodium sulfate, separated and purified by column chromatography (dichloromethane:methanol=15:1), then concentrated and dried to obtain the yellow solid of desired product K-092, MS: 705.32 [M+H]⁺

### Example 93: Synthesis of Compound K-093

### (4-(5-Bromo-2-((5-ethyl-2-methoxy-4-(4-morpholinopiperidin-1-yl)phenyl)amino)pyrimidin-4-yl)amino)-2′-fluoro-[1,1′-biphenyl]-3-yl)dimethylphosphine oxide

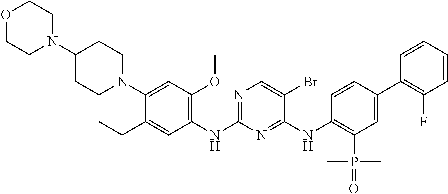 [SMILES: CCc1cc(Nc2ncc(Br)c(Nc3ccc(-c4ccccc4F)cc3P(C)(C)=O)n2)c(OC)cc1N1CCC(N2CCOCC2)CC1]

### Step 1: Synthesis of Compound K-093-1

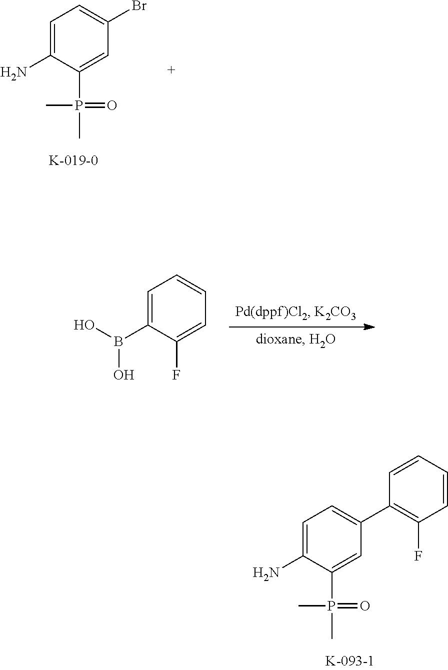 [SMILES: CP(C)(=O)c1cc(Br)ccc1N ; OB(O)c1ccccc1F ; "Pd(dppf)Cl2, K2CO3" ; "dioxane, H2O" ; CP(C)(=O)c1cc(-c2ccccc2F)ccc1N]

The compound K-093-1 was synthesized as the method described for compound K-019-1 using 2-Fluorophenylboronic acid instead of bis(pinacolato)diboron. For compound K-093-1, MS: 264.09 [M+H]⁺

### Step 2: Synthesis of Compound K-093-2

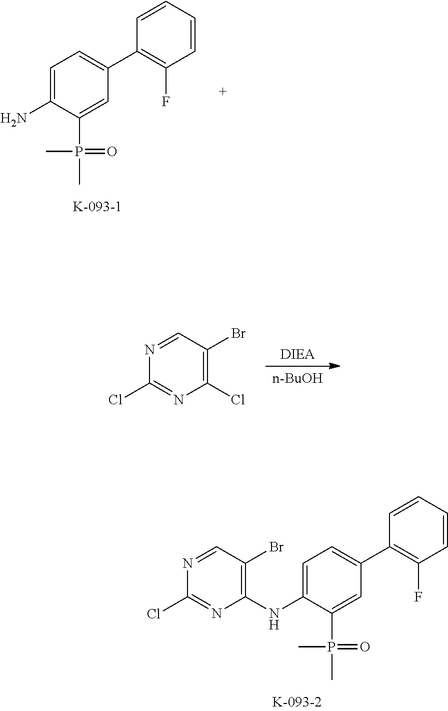

The compound K-093-2 was synthesized as the method described for compound K-019-3 using compound K-093-1 instead of compound K-019-2. For compound K-093-2, MS: 453.98 [M+H]⁺

### Step 3: Synthesis of Compound K-093-3

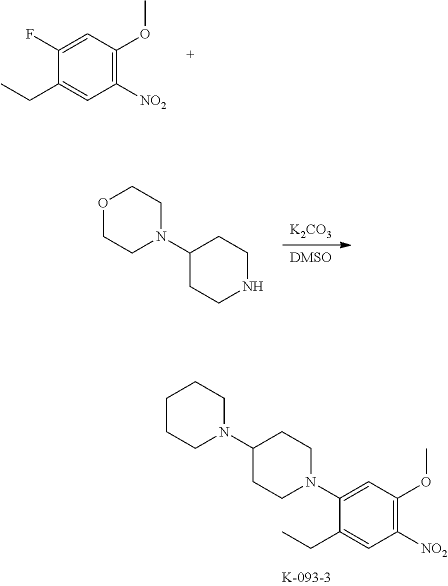

The compound K-093-3 was synthesized as the method described for compound ns K-024-1 using 4-Morpholinopiperidine and 1-Ethyl-2-fluoro-4-methoxy-5-nitrobenzene instead of two raw compounds. For compound K-093-3, MS: 350.20 [M+H]⁺

### Step 4: Synthesis of Compound K-093-4

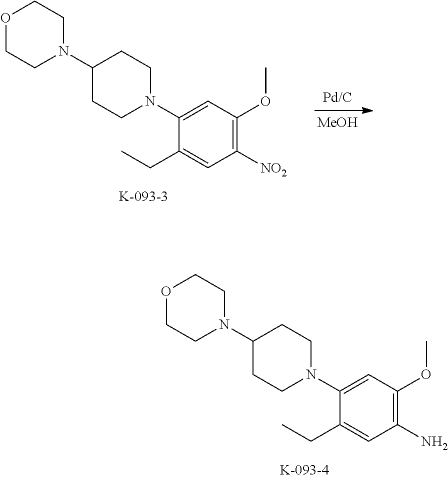 [SMILES: "MeOH" ; CCc1cc([N+](=O)[O-])c(OC)cc1N1CCC(N2CCOCC2)CC1 ; CCc1cc(N)c(OC)cc1N1CCC(N2CCOCC2)CC1]

The compound K-093-4 was synthesized as the method described for compound K-024-2 using compound K-093-3 instead of raw compound K-024-1. For compound K-093-4, MS: 320.23 [M+H]⁺

### Step 5: Synthesis of Compound K-093

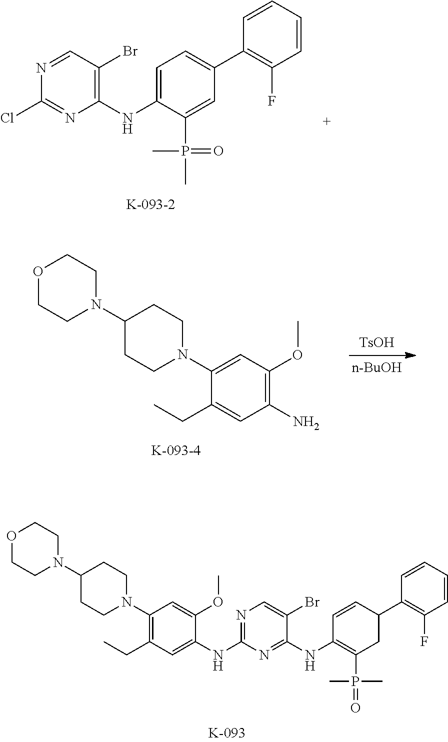

The compound K-093 was synthesized as the method described for compound K-019 using compound K-093-4 instead of raw compound K-018-9, compound K-093-2 instead of compound K-019-3. For compound K-093, MS: 737.23 [M+H]⁺

### Example 94: Synthesis of Compound K-094

### (2-(5-Bromo-2-((5-ethyl-2-methoxy-4-(4-morpholinopiperidin-1-yl)phenyl)amino)pyrimidin-4-yl)amino)-5-(2-methyl-2H-1,2,3-triazol-4-yl)phenyl)dimethylphosphine oxide

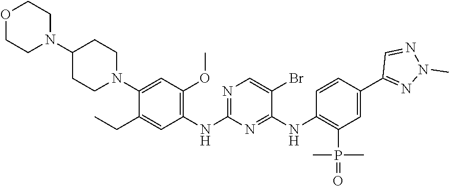 [SMILES: CCc1cc(Nc2ncc(Br)c(Nc3ccc(-c4cnn(C)n4)cc3P(C)(C)=O)n2)c(OC)cc1N1CCC(N2CCOCC2)CC1]

### Step 1: Synthesis of Compound K-094

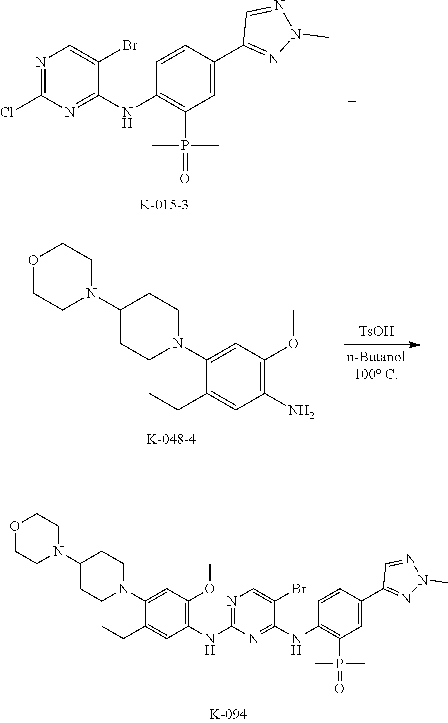 [SMILES: Cn1ncc(-c2ccc(Nc3nc(Cl)ncc3Br)c(P(C)(C)=O)c2)n1 ; CCc1cc(N)c(OC)cc1N1CCC(N2CCOCC2)CC1 ; "TsOH" ; "n-Butanol100° C." ; CCc1cc(Nc2ncc(Br)c(Nc3ccc(-c4cnn(C)n4)cc3P(C)(C)=O)n2)c(OC)cc1N1CCC(N2CCOCC2)CC1]

The compound K-094 was synthesized as the method described for compound K-018 using compound K-093-4 instead of raw compound K-018-9, compound K-015-3 instead of raw compound K-018-5. For compound K-094, MS: 724.24 [M+H]⁺

### Example 95: Synthesis of Compound K-095

### (5-(2-Aminothiazol-4-yl)-2-((5-chloro-2-((5-ethyl-2-methoxy-4-(4-(4-methylpiperazin-1-yl)piperidin-1-yl)phenyl)amino)pyrimidin-4-yl)amino)phenyl)dimethylphosphine oxide

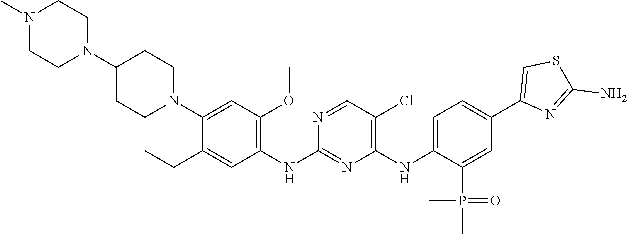 [SMILES: CCc1cc(Nc2ncc(Cl)c(Nc3ccc(-c4csc(N)n4)cc3P(C)(C)=O)n2)c(OC)cc1N1CCC(N2CCN(C)CC2)CC1]

### Step 1: Synthesis of Compound K-095

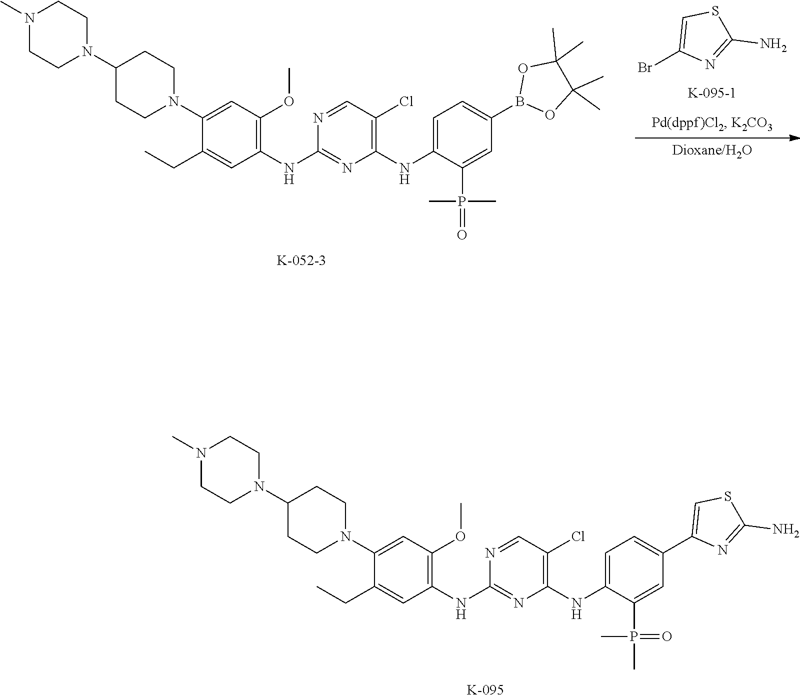

The compound K-095 was synthesized as the method described for compound K-060 using compound K-095-1 instead of raw compound K-060-2. For compound K-095, MS: 710.28 [M+H]⁺

### Example 96: Synthesis of Compound K-096

### (2-((5-Bromo-2-((2-methoxy-4-(4-(4-methylpiperazin-1-vi)piperidin-1-yl)-5-(tetrahydro-2H-pyran-4-yl)phenyl)amino)pyrimidin-4-yl)amino)-5-(thiazol-4-yl)phenyl)dimethylphosphine oxide

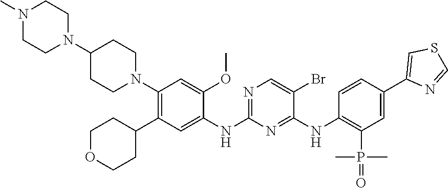 [SMILES: COc1cc(N2CCC(N3CCN(C)CC3)CC2)c(C2CCOCC2)cc1Nc1ncc(Br)c(Nc2ccc(-c3cscn3)cc2P(C)(C)=O)n1]

### Step 1: Synthesis of Compound K-096-1

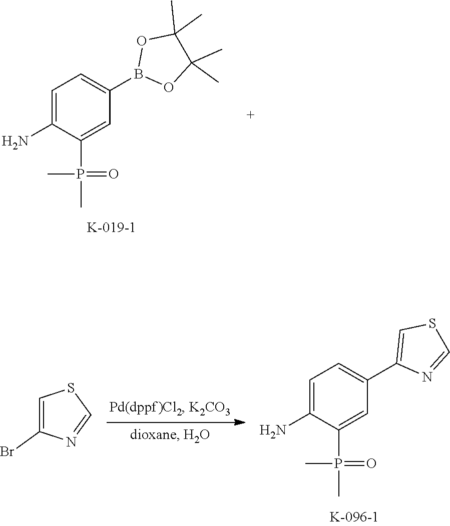 [SMILES: CC1(C)OB(c2ccc(N)c(P(C)(C)=O)c2)OC1(C)C ; Brc1cscn1 ; CP(C)(=O)c1cc(-c2cscn2)ccc1N]

The compound K-096-1 was synthesized as the method described for compound K-019-2 using 4-Bromothiazolc instead of 2-Bromothiazole. For compound K-096-1, MS: 253.05 [M+H]⁺

### Step 2: Synthesis of Compound K-096-2

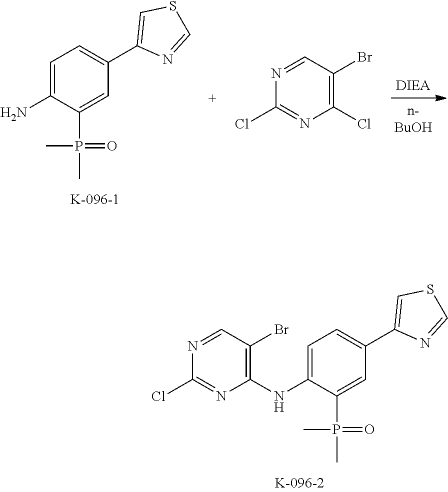 [SMILES: CP(C)(=O)c1cc(-c2cscn2)ccc1N ; Clc1ncc(Br)c(Cl)n1 ; CP(C)(=O)c1cc(-c2cscn2)ccc1Nc1nc(Cl)ncc1Br]

The compound K-096-2 was synthesized as the method described for compound K-019-3 using compound K-096-1 instead of raw compound K-019-2. For compound K-096-2, MS: 442.94 [M+H]⁺

### Step 3: Synthesis of Compound K-096-3

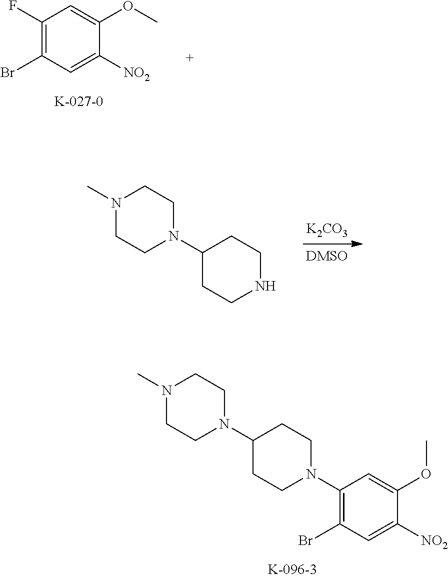

Compound K-096-3 was synthesized as the method described for compound K-024-1 using compound K-027-0 instead of raw compound K-024-0, 1-Methyl-4-(piperidin-4-yl)piperazine instead of morpholine. For compound K-096-3, MS: 413.11 [M+H]⁺

### Step 4: Synthesis of Compound K-096-4

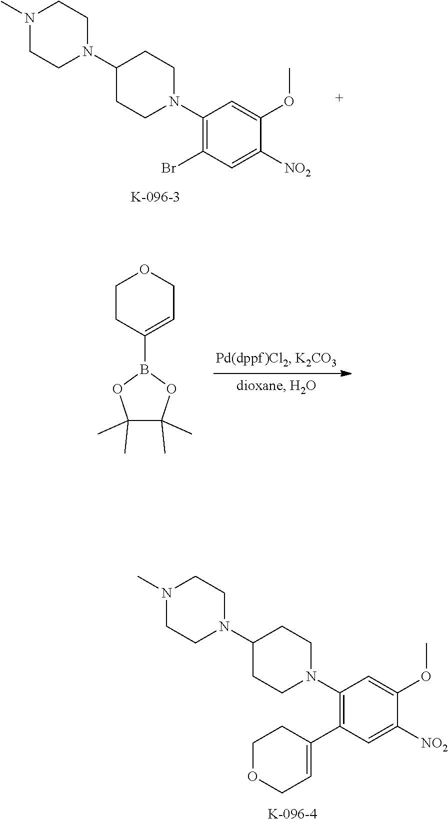

The compound K-096-4 was synthesized as the method described for compound K-019-2 using 3,6-Dihydro-2H-pyran-4-boronic acid pinacol ester instead of 2-Bromothiazole, compound K-096-3 instead of compound K-019-1. For compound K-096-4, MS: 417.24 [M+H]⁺

### Step 5: Synthesis of Compound K-096-5

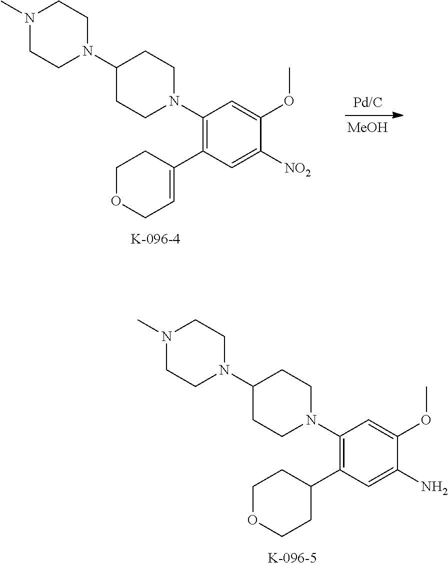 [SMILES: "MeOH" ; COc1cc(N2CCC(N3CCN(C)CC3)CC2)c(C2=CCOCC2)cc1[N+](=O)[O-] ; COc1cc(N2CCC(N3CCN(C)CC3)CC2)c(C2CCOCC2)cc1N]

The compound K-096-5 was synthesized as the method described for compound K-024-2 using compound K-096-4 instead of raw compound K-024-1. For compound K-096-5, MS: 389.28 [M+H]⁺

### Step 6: Synthesis of Compound K-096

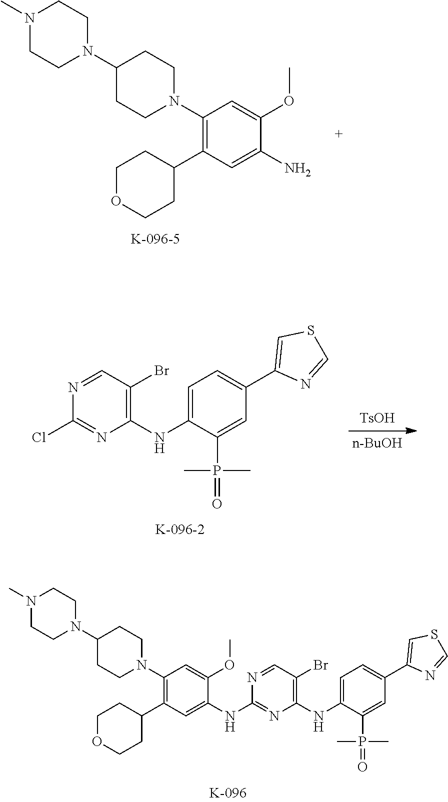

Add compounds (92.8 mg, 210.54 umol), K-096-5 (73.0 mg, 190.0 umol), p-toluenesulfonic acid (65.0 mg, 378.0 umol) and n-BuOH (1 mL) to the reaction flask, stir at 120° C. overnight. The reaction was complete as monitored by LCMS. The reaction solution was cooled down to room temperature, and spin-dried. Saturated Na₂CO₃ aqueous solution (10 mL) was added to the reaction solution. And then the solution was extracted with dichloromethane (3×10 mL), dried over anhydrous sodium sulfate, and then filtered and spin-dried. The crude product was purified by PTLC (dichloromethane:methanol=10:1) to obtain the desired compound K-096, MS: 795.25 [M+H]⁺

### Example 97: Synthesis of Compound K-097

### (2-((5-Bromo-2-((5-ethyl-2-methoxy-4-(4-(4-methylpiperazin-1-yl)piperidin-1-yl)phenyl)amino)amino]pyrimidin-4-yl)amino)-5-(6-hydroxypyridazin-3-yl)phenyl)dimethylphosphine oxide

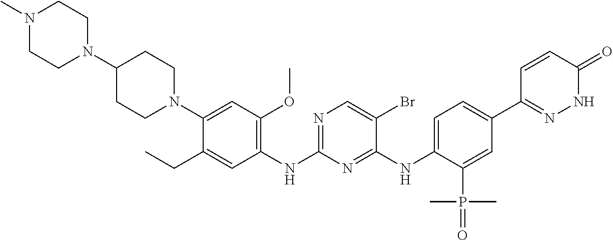 [SMILES: CCc1cc(Nc2ncc(Br)c(Nc3ccc(-c4ccc(=O)[nH]n4)cc3P(C)(C)=O)n2)c(OC)cc1N1CCC(N2CCN(C)CC2)CC1]

### Step 1: Synthesis of Compound K-097-2

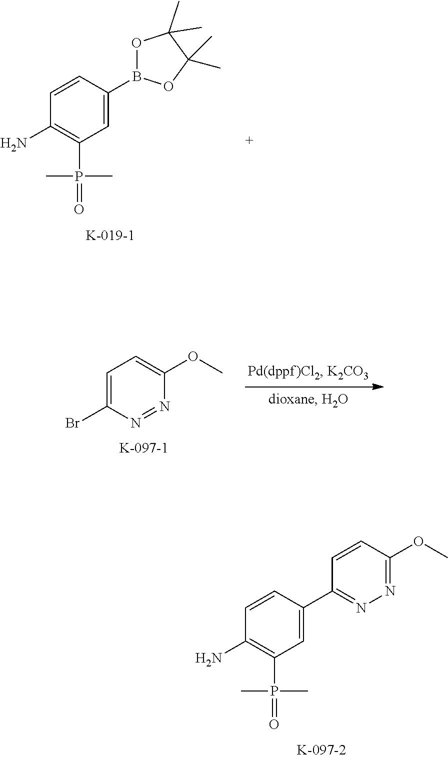

The compound K-097-2 was synthesized as the method described for compound K-019-2 using compound K-097-1 instead of 2-Bromothiazole. For compound K-097-2, MS: 278.10 [M+H]⁺

### Step 2: Synthesis of Compound K-097-3

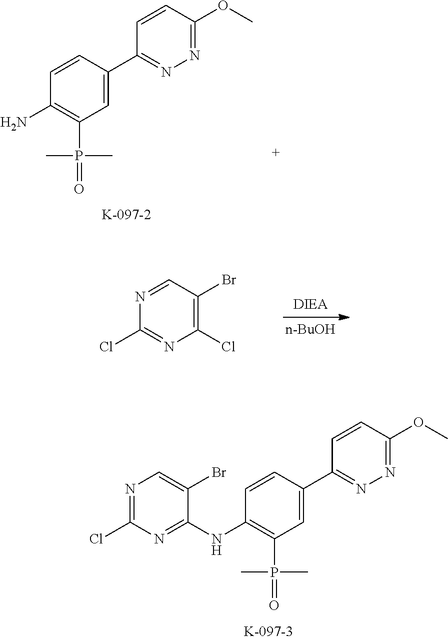

The compound K-097-3 was synthesized as the method described for compound K-019-3 using compound K-097-2 instead of compound K-019-2. For compound K-097-3, MS: 467.99 [M+H]⁺

### Step 3: Synthesis of Compound K-097

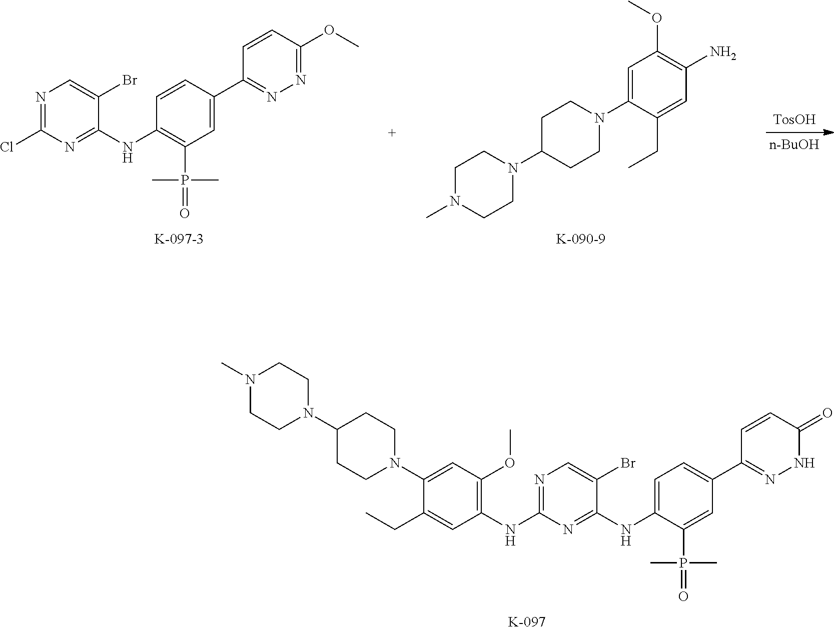

The compound K-097 was synthesized as the method described for compound K-090 using compound K-097-3 instead of raw compound K-090-5. For compound K-097, MS: 750.26 [M+H]⁺

### Example 98: Synthesis of Compound K-098

### (5-(3-Aminopyridin-2-yl)-2-((5-bromo-2-((5-ethyl-2-methoxy-4-(4-(4-methylpiperazin-1-yl)piperidin-1-yl)phenyl)amino)pyrimidin-4-yl)amino)phenyl)dimethylphosphine oxide

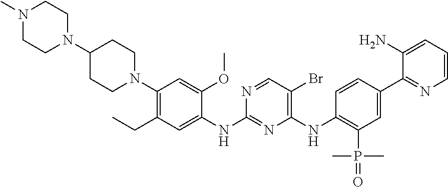 [SMILES: CCc1cc(Nc2ncc(Br)c(Nc3ccc(-c4ncccc4N)cc3P(C)(C)=O)n2)c(OC)cc1N1CCC(N2CCN(C)CC2)CC1]

### Step 1: Synthesis of Compound K-098

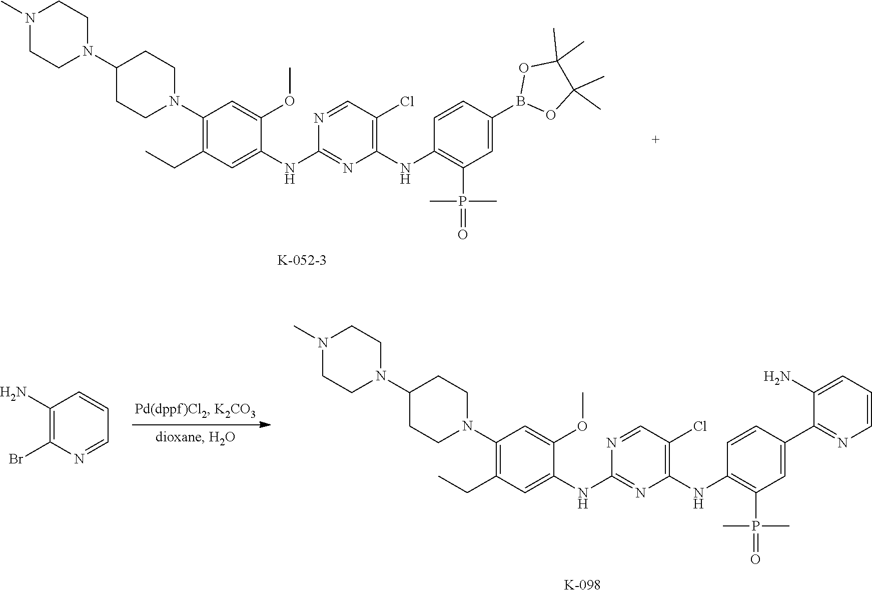

Add compound K-052-3 (70 mg), 2-Bromo-3-aminopyrimidine (30 mg), Pd(dppf)Cl₂ (12 mg), K₂CO₃ (40 mg), dioxane (4 mL) and water (1 mL) successively to the reaction flask, heat to 100° C. under nitrogen protection and stir with heating for 12 hr. The reaction was complete as monitored by LCMS, and then stopped. After removing the solvent, the residue was separated and purified by the preparative TLC (dichloromethane:methanol=6:1) to obtain the yellowish solid of desired compound 14 (22.3 mg), MS: 704.33 [M+H]+

### Example 99: Synthesis of Compound K-099

### (2-((5-Bromo-2-((5-ethyl-2-methoxy-4-(4-(4-methylpiperazin-1-yl)piperidin-1-yl)phenyl)amino)amino]pyrimidin-4-yl)amino)-5-(4-hydroxypyrimidin-5-yl)phenyl)dimethylphosphine oxide

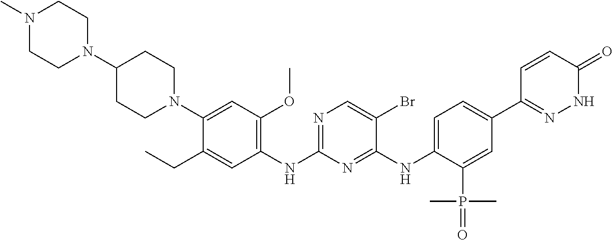 [SMILES: CCc1cc(Nc2ncc(Br)c(Nc3ccc(-c4ccc(=O)[nH]n4)cc3P(C)(C)=O)n2)c(OC)cc1N1CCC(N2CCN(C)CC2)CC1]

### Step 1: Synthesis of Compound K-099-2

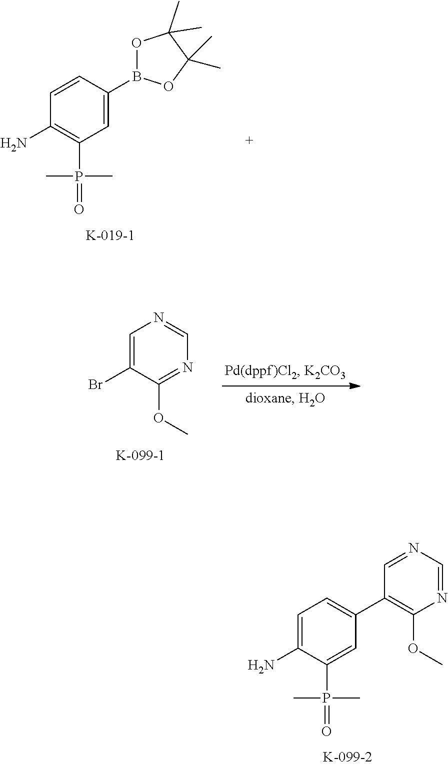

The compound K-099-2 was synthesized as the method described for compound K-019-2 using compound K-099-1 instead of 2-Bromothiazole. For compound K-099-2, MS: 278.10 [M+H]⁺

### Step 2: Synthesis of Compound K-099-3

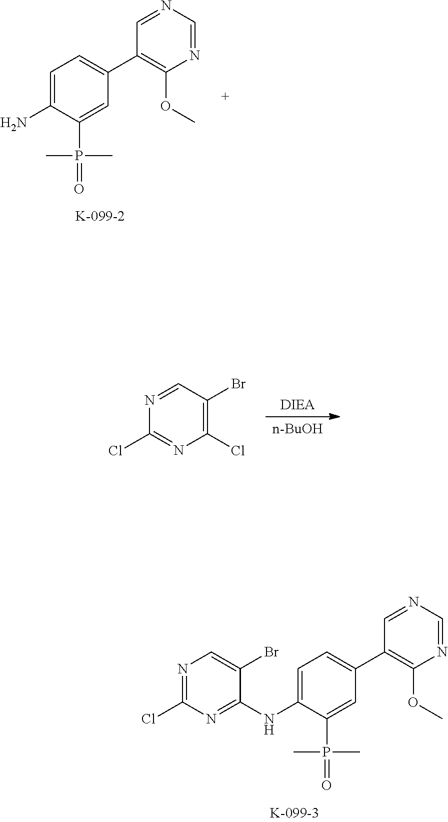

The compound K-099-3 was synthesized as the method described for compound K-019-3 using compound K-099-2 instead of raw compound K-019-2. For compound K-099-3, MS: 467.99 [M+H]⁺

### Step 3: Synthesis of Compound K-099

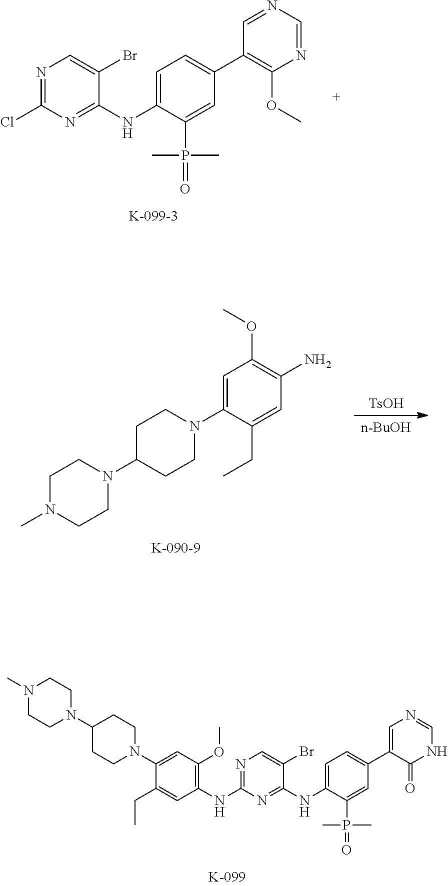

The compound K-099-3 was synthesized as the method described for compound K-090 using compound K-099-3 instead of compound K-090-5. For compound K-099, MS: 750.26 [M+H]⁺

### Example 100: Synthesis of Compound K-100

### (4-((2-((4-(4-([1,4′-Bipiperidin]-1′-yl)-2-methoxy-5-(1-methyl-1H-pyrazole-4-yl)phenyl)amino)-5-bromopyrimidin-4-yl)amino)-[1,1′-biphenyl]-3-yl)dimethylphosphine oxide

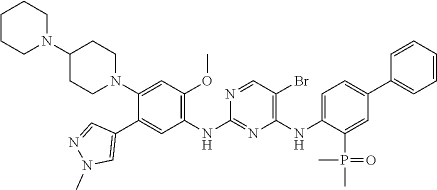 [SMILES: COc1cc(N2CCC(N3CCCCC3)CC2)c(-c2cnn(C)c2)cc1Nc1ncc(Br)c(Nc2ccc(-c3ccccc3)cc2P(C)(C)=O)n1]

### Step 1: Synthesis of Compound K-100

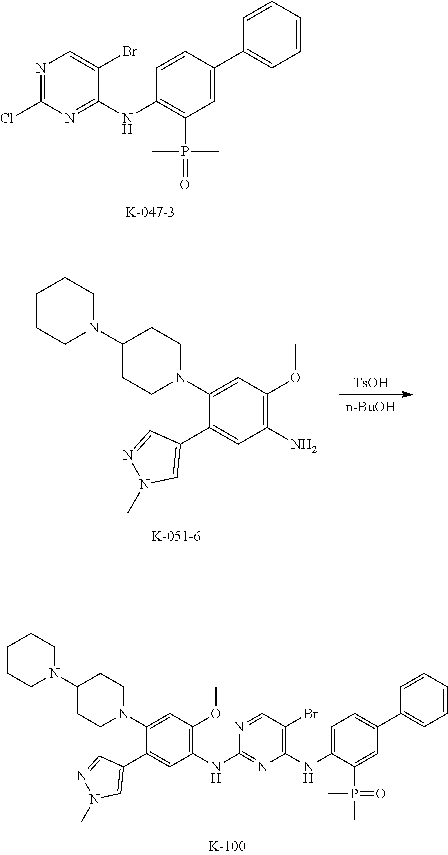

The compound K-100 was synthesized as the method described for compound K-047 using compound K-051-6 instead of raw compound K-093-4. For compound K-100, MS: 769.27 [M+H]⁺

### Example 101: Synthesis of Compound K-101

### (2-((5-Bromo-2-((5-ethyl-2-methoxy-4-(4-(4-methylpiperazin-1-yl)piperidin-1-yl)phenyl)aminopyrimidin-4-yl)amino)-5-(1,5-dimethyl-1H-pyrazol-3-yl)phenyl)dimethylphosphine oxide

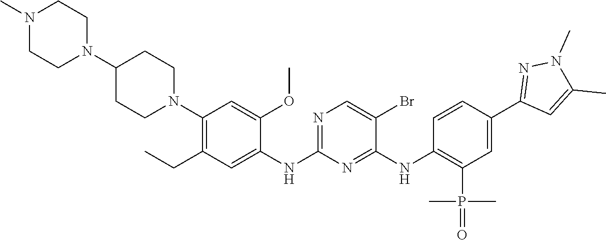

### Step 1: Synthesis of Compound K-101

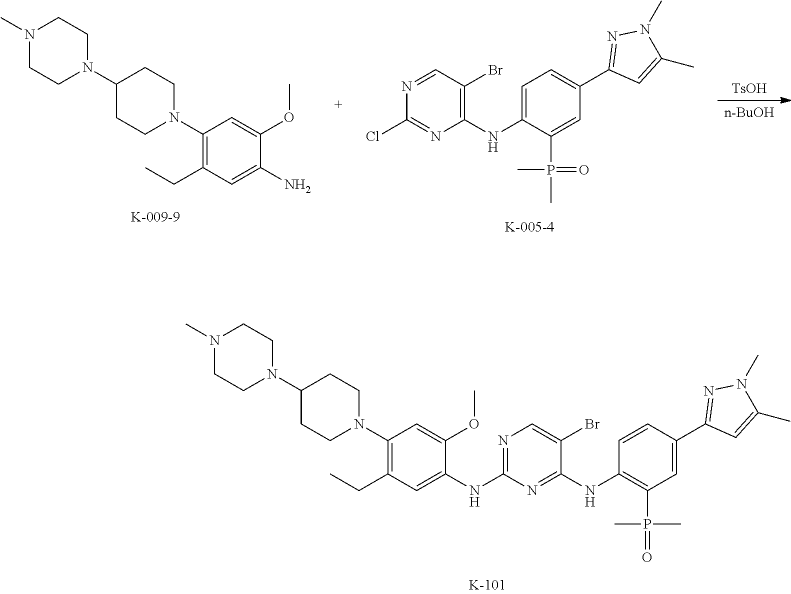

The compound K-101 was synthesized as the method described for compound K-018 using compound K-009-9 instead of raw compound K-018-9, compound K-005-4 instead of raw compound K-018-5. For compound K-101, MS: 750.29 [M+H]⁺

### Example 102: Synthesis of Compound K-102

### (2-((5-Bromo-2-((5-(1-ethyl-1H-pyrazol-4-yl)-2-methoxy-4-(4-(4-methylpiperazin-1-yl)piperidin-1-yl)phenyl)amino)pyrimidin-4-yl)amino)-5-(1-methyl-1H-pyrazolo[3,4-b]pyridin-3-yl)phenyl)dimethylphosphine oxide

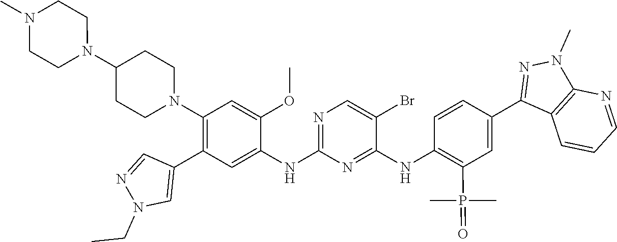

### Step 1: Synthesis of Compound K-102

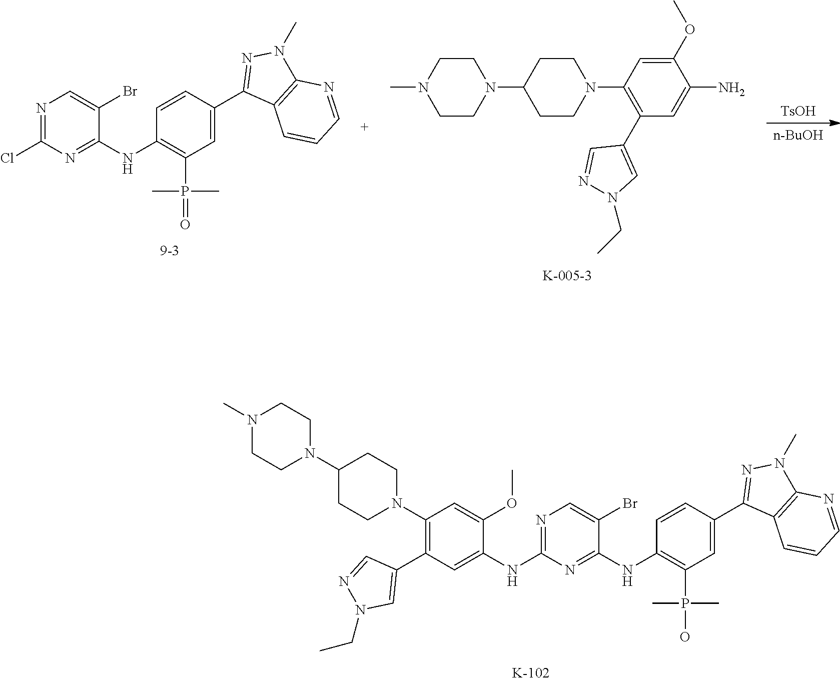

The compound K-102 was synthesized as the method described for compound K-018 using compound K-005-3 instead of raw compound K-018-9, compound 9-3 instead of raw compound K-018-5. For compound K-102, MS: 853.31 [M+H]⁺

### Example 103: Synthesis of Compound K-103

### 5-(3-Aminopyridin-2-yl)-2-((5-chloro-2-((5-ethyl-2-methoxy-4-(4-(4-methylpiperazin-1-yl)piperidin-1-yl)phenyl)amino)pyrimidin-4-yl)amino)phenyl)dimethylphosphine oxide

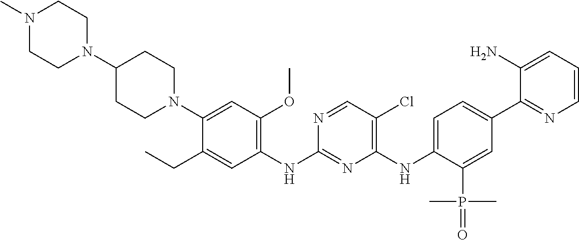 [SMILES: CCc1cc(Nc2ncc(Cl)c(Nc3ccc(-c4ncccc4N)cc3P(C)(C)=O)n2)c(OC)cc1N1CCC(N2CCN(C)CC2)CC1]

### Step 1: Synthesis of Compound K-103

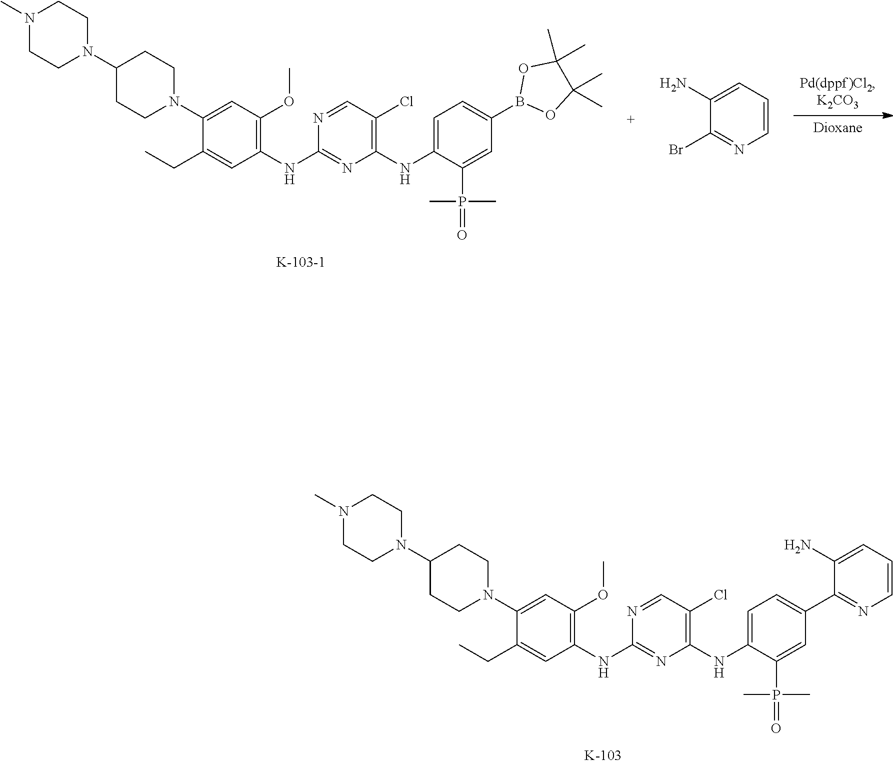

Add compound K-103-1, 2-Bromo-3-aminopyrimidine, Pd(dppf)Cl₂, K₂CO₃, dioxane and water successively to the reaction flask, heat to 110° C. under nitrogen protection, and stir with heating for 16 hr. The reaction was complete as monitored by LCMS, and then stopped. The reaction solution was poured into water (50 mL), and extracted with dichloromethane (3×50 mL); the organic phase was washed with saturated brine (3×30 mL), dried over anhydrous sodium sulfate, separated and purified by column chromatography (dichloromethane:methanol=15:1), then concentrated and dried to obtain the yellow solid of desired product K-103, MS: 704.33 [M+H]⁺

### Example 104: Synthesis of Compound K-104

### (2-((5-Chloro-2-((5-ethyl-2-methoxy-4-(4-(4-methylpiperazin-1-yl)piperidin-1-yl)phenyl)amino)pyrimidin-4-yl)amino)-5-(4,5,6,7-tetrahydropyrazolo[1,5-a]pyridin-3-yl)phenyl)dimethylphosphine oxide

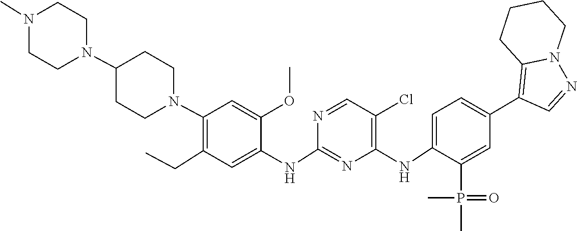 [SMILES: CCc1cc(Nc2ncc(Cl)c(Nc3ccc(-c4cnn5c4CCCC5)cc3P(C)(C)=O)n2)c(OC)cc1N1CCC(N2CCN(C)CC2)CC1]

### Step 1: Synthesis of Compound K-104

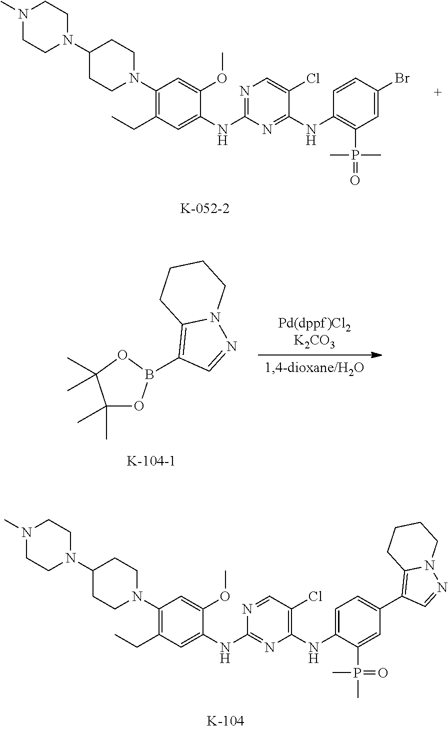 [SMILES: CCc1cc(Nc2ncc(Cl)c(Nc3ccc(Br)cc3P(C)(C)=O)n2)c(OC)cc1N1CCC(N2CCN(C)CC2)CC1 ; CC1(C)OB(c2cnn3c2CCCC3)OC1(C)C ; "1,4-dioxane/H2O" ; "Pd(dppf)Cl2K2CO3" ; CCc1cc(Nc2ncc(Cl)c(Nc3ccc(-c4cnn5c4CCCC5)cc3P(C)(C)=O)n2)c(OC)cc1N1CCC(N2CCN(C)CC2)CC1]

Dissolve compound K-052-2 (50 mg) in 1,4-dioxane (5 ml) and water (1 ml) in a 50 ml stand-up bottle, and then successively add K-104-1 (20 mg), Pd (dppf)Cl₂ (6 mg) and anhydrous potassium carbonate (30 mg). The reaction system was replaced with nitrogen, then heated to 100° C. and stirred overnight. It was naturally cooled down to room temperature, and the solution was diluted with water (20 ml), extracted twice with dichloromethane (20 ml*2), combined with organic phase, dried over anhydrous sodium sulfate, filtered and concentrated under reduced pressure. The residue was dissolved in a small amount of dichloromethane, loaded with samples and purified on a thick preparation plate (DCM/MeOH=10/1) to obtain compound K-104 (22.7 mg), MS: 732.36 [M+H]⁺

The compounds in example 105-106 were synthesized as the method described in example 104, except that the raw materials replaced.

| | | | Molecular |
|---|---|---|---|
| | | | weight |
| Example | Structural formula | Name | [M + H]⁺ |
| Example 105 (compound K-105) | 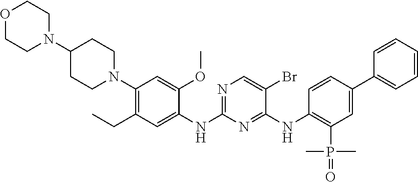 [SMILES: CCc1cc(Nc2ncc(Br)c(Nc3ccc(-c4ccccc4)cc3P(C)(C)=O)n2)c(OC)cc1N1CCC(N2CCOCC2)CC1] | (4-((5-Bromo-2-((5-ethyl-2-methoxy-4- (4-morpholinopiperidin-1-yl)phenyl) amino)pyrimidin-4-yl)amino)-[1,1′- biphenyl]-3-yl)dimethylphosphine oxide | 719.24 |
| Example 106 (compound K-106) | 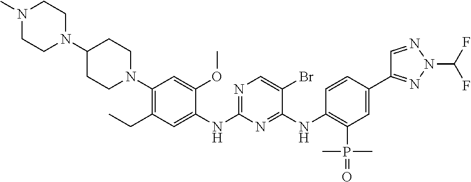 [SMILES: CCc1cc(Nc2ncc(Br)c(Nc3ccc(-c4cnn(C(F)F)n4)cc3P(C)(C)=O)n2)c(OC)cc1N1CCC(N2CCN(C)CC2)CC1] | (2-((5-Bromo-2-((5-ethyl-2-methoxy-4- (4-(4-methylpiperazin-1-yl)piperidin-1- yl)phenyl)amino)pyrimidin-4-yl)amino)- 5-(2-(difluoromethyl)-2H-1,2,3-triazol- 4-yl)phenyl)dimethylphosphine oxide | 773.25 |

### Example 107: Synthesis of Compound 1

### (4-((5-Bromo-2-((5-ethyl-2-methoxy-4-(4-methylpiperazin-1-yl)piperidin-1-yl)phenyl)amino)pyrimidin-4-yl)amino)pyrimidin-4-yl)amino)-[1,1′-biphenyl]-3-yl)dimethylphosphino oxide

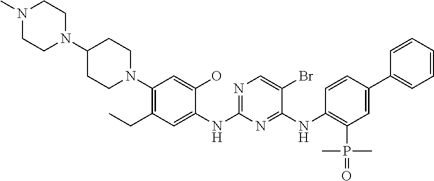

### Step 1: Synthesis of Compound 1-3

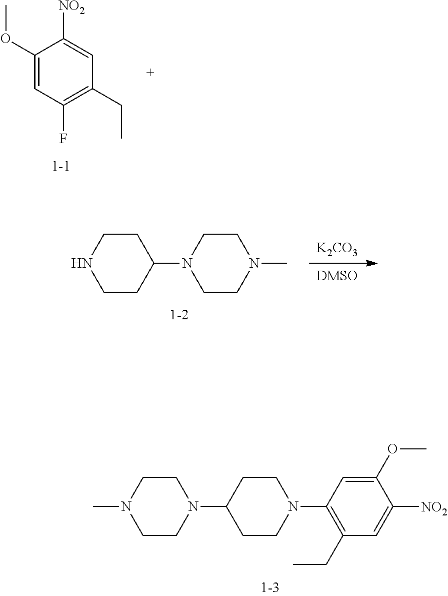

Add compounds 1-1 (3.00 g), 1-2 (4.14 g), K₂CO₃ (6.24 g) and DMSO (30 mL) successively to the reaction flask, heat to 90° C., and stir with heating for 12 hr. The reaction was complete as monitored by LCMS, and then stopped. The reaction solution was poured into water (100 mL), filtered with suction, and the filter cake was washed with water and dried to obtain the yellow solid of desired product 1-3 (4.20 g), MS: 363.23 [M+H]⁺

### Step 2: Synthesis of Compound 1-4

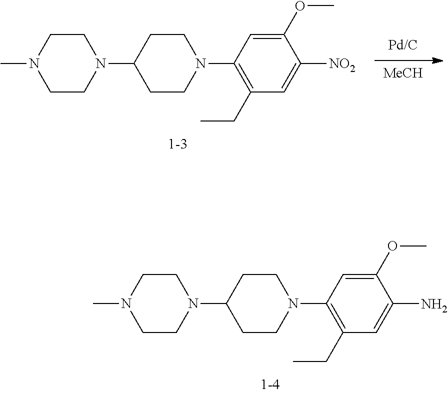

Add compound 1-3 (4.20 g), Pd/C (1.00 g) and MeOH (60 ml) successively to the reaction flask, introduce H₂; the reaction solution was stirred at room temperature for 3 hr. The reaction was complete as monitored by LCMS, and then stopped. The solution was filtered with suction, and rinsed with methanol (20 mL); collect the organic phase, remove the solvent, and obtain the reddish-brown liquid of desired product compound 1-4 (3.5 g), MS: 333.26 [M+H]⁺

### Step 3: Synthesis of Compound 1-6

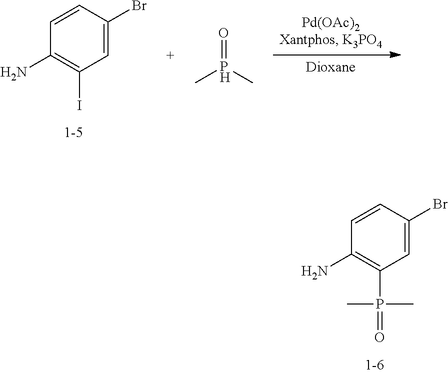

Add compound 1-5 (3.0 g), dimethylphosphine oxide (865 mg), K₃PO₄ (6.41 g), Pd(OAc)₂ (226 mg), Xantphos (1.75 g) and dioxane (60 mL) successively to the reaction flask, heat to 100° C. under nitrogen protection, and stir with heating for 4 hr. The reaction was complete as monitored by LCMS, and then stopped. Add water (50 mL) into the reaction solution. And then the reaction solution was extracted with dichloromethane (3×50 mL); the organic phase was washed with saturated brine (3×30 mL), dried over anhydrous sodium sulfate, separated and purified by column chromatography (dichloromethane:methanol=15:1), and the solvent was removed to obtain brown solid of desired product 1-6 (2.0 g), MS: 247.98 [M+H]⁺

### Step 4: Synthesis of Compound 1-8

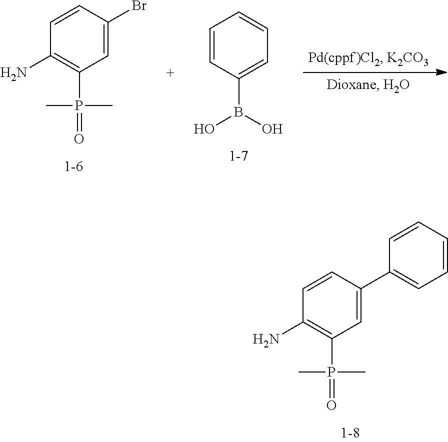 [SMILES: CP(C)(=O)c1cc(Br)ccc1N ; OB(O)c1ccccc1 ; "Dioxane, H2O" ; "Pd(cppf)Cl2, K2CO3" ; CP(C)(=O)c1cc(-c2ccccc2)ccc1N]

Add compounds 1-6 (1.0 g), 1-7 (590 mg), K₂CO₃ (1.67 g), Pd(dppf)Cl₂ (295 mg), dioxane (20 ml) and water (4 ml) successively to the reaction flask, heat to 100° C. under nitrogen protection, and stir with heating for 12 hr. The reaction was complete as monitored by LCMS, and then stopped. Add water (50 ml) into the reaction solution. And then the reaction solution was extracted with dichloromethane (3×50 mL); the organic phase was washed with saturated brine (3×30 mL), dried over anhydrous sodium sulfate, separated and purified by column chromatography (dichloromethane:methanol=10:1), and the solvent was removed to obtain the brown solid of desired product 1-8 (900 mg), MS: 246.10 [M+H]+

### Step 5: Synthesis of Compound 1-10

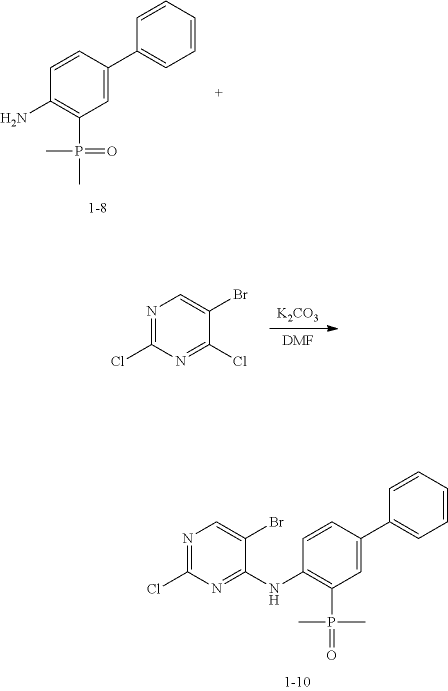

Add compound 1-8 (1.0 g), 5-Bromo-2, 4-dichloropyrimidine (1.39 g), K₂CO₃ (1.69 g) and DMF (20 ml) successively to the reaction flask, heat to 100° C. under nitrogen protection, and stir with heating for 12 hr. The reaction was complete as monitored by LCMS, and then stopped. Add water (50 ml) into the reaction solution. And then the reaction solution was extracted with ethyl acetate (3×50 mL); the organic phase was washed with saturated brine (3×30 mL), dried over anhydrous sodium sulfate, separated and purified by column chromatography (dichlormethane:methanol=13:1), and the solvent was removed to obtain the yellow solid of desired product compound 1-10 (700 mg), MS: 435.99 [M+H]⁺

### Step 6: Synthesis of Compound 1

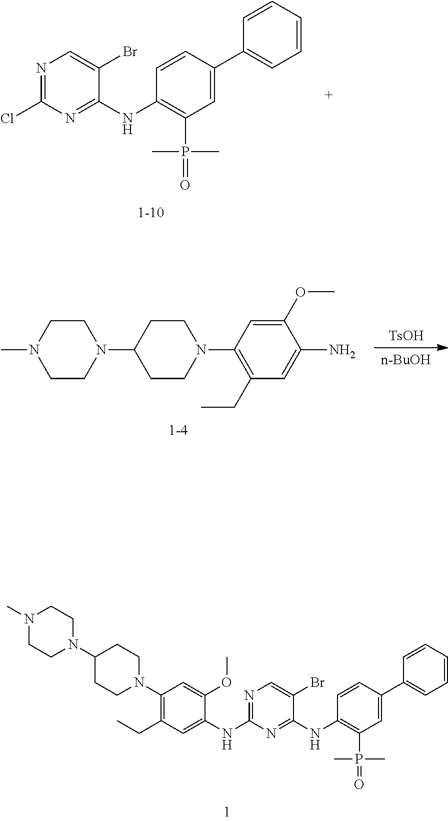

Add compounds 1-10 (100 mg), 1-4 (76 mg), p-toluenesulfonic acid (60 mg) and n-butanol (2 mL) successively into the reaction flask, heat to 120° C., and stir for 12 hr. The reaction was complete as monitored by LCMS, and then stopped. The reaction solution was poured into sodium carbonate solution (5 mL), and extracted with dichloromethane (3×50 mL); the organic phase was washed with saturated brine (3×30 mL), dried over anhydrous sodium sulfate, separated and purified by column chromatography (dichloromethane:methanol=12:1), and then concentrated to obtain off-white solid of compound 1 (60 mg), MS: 732.27 [M+H]⁺

### Example 108: Synthesis of Compound 2

### (4-((5-Chloro-2-((5-ethyl-2-methoxy-4-(4-(4-methylpiperazin-1-yl)piperidin-1-yl)phenyl)amino)pyrimidin-4-yl)amino)-[1,1′-biphenyl]-3-yl)dimethylphosphine oxide

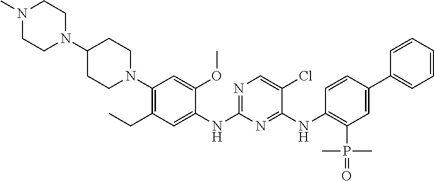 [SMILES: CCc1cc(Nc2ncc(Cl)c(Nc3ccc(-c4ccccc4)cc3P(C)(C)=O)n2)c(OC)cc1N1CCC(N2CCN(C)CC2)CC1]

### Step 1: Synthesis of Compound 2-2

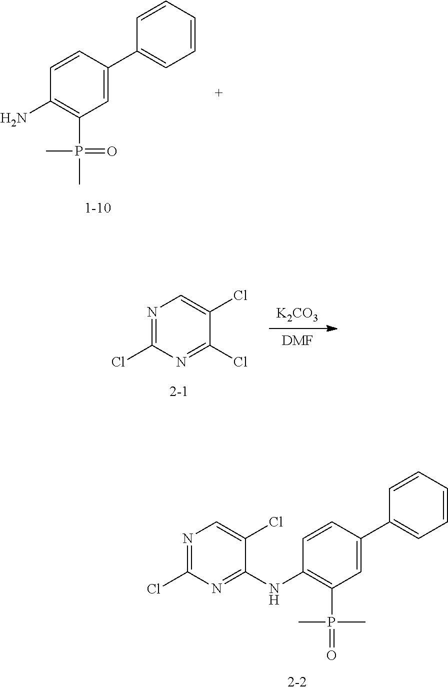

The compound 2-2 was synthesized as the method described for compound 1-10 using compound 2-1 instead of raw compound 5-Bromo-2,4-dichloropyrimidine. For compound 2-2, MS: 392.04 [M+H]⁺

### Step 2: Synthesis of Compound 2

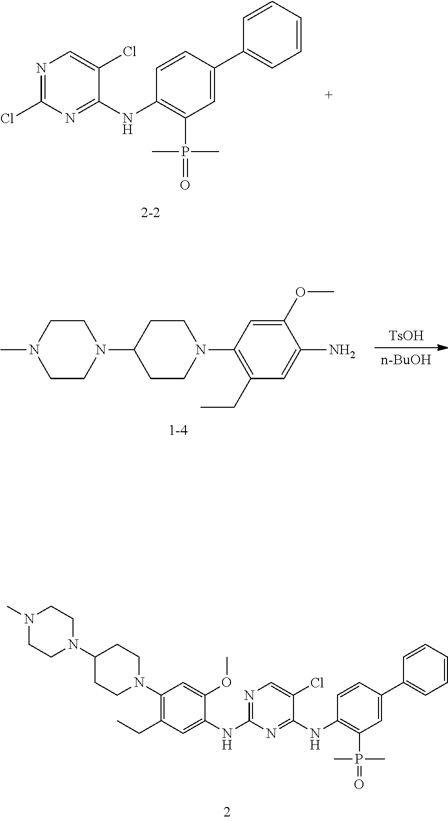

The compound 2 was synthesized as the method described for compound 1 using compound 2-2 instead of raw compound 1-10. For compound 2, MS: 688.32 [M+H]⁺

### Example 109: Synthesis of Compound 3

### (2-((5-Bromo-2-((5-ethyl-2-methoxy-4-(4-(4-methylpiperazin-1-yl)piperidin-1-yl)phenyl)amino)pyrimidin-4-yl)amino)-5-(1-methyl-1hydro-pyrazol-4-yl)phenyl)dimethylphosphine oxide

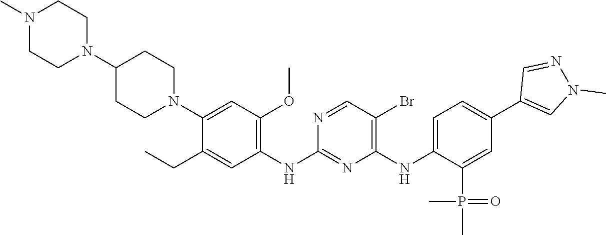

### Step 1: Synthesis of Compound 3-2

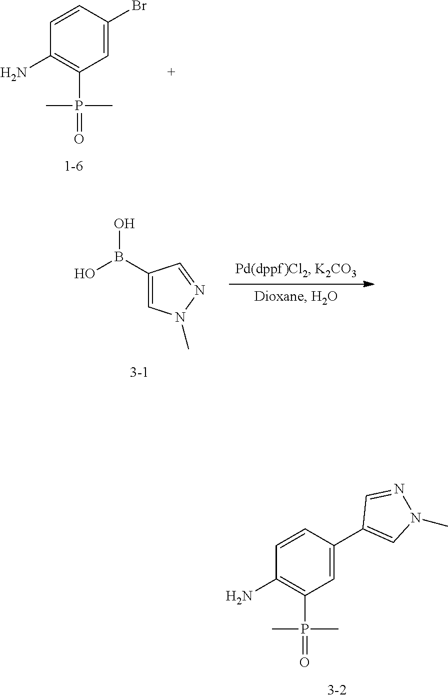

The compound 3-2 was synthesized as the method described for compound 1-8 using compound 3-1 instead of raw compound 1-7. For compound 3-2, MS: 250.10 [M+H]⁺

### Step 2: Synthesis of Compound 3-3

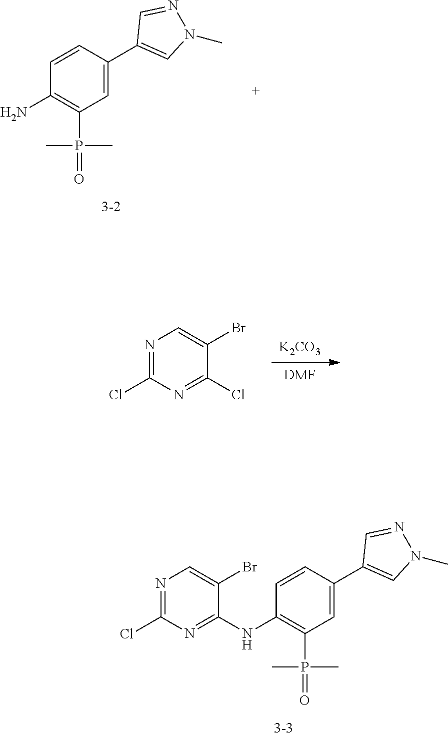

The compound 3-3 was synthesized as the method described for compound 1-10 using compound 3-2 instead of raw compound 1-8. For compound 3-3, MS: 440.00 [M+H]⁺

### Step 3: Synthesis of Compound 3

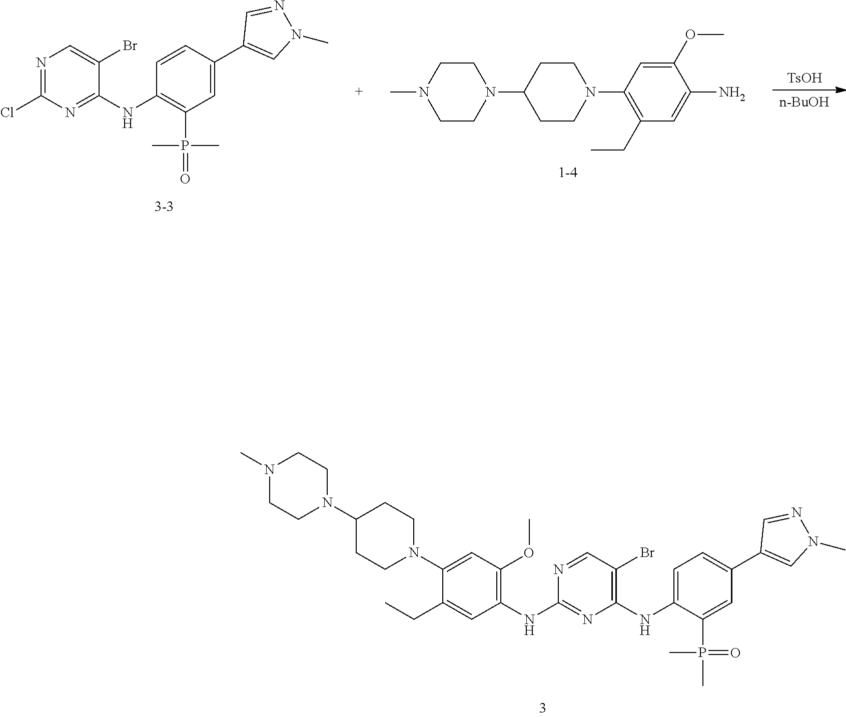

The compound 3 was synthesized as the method described for compound 1 using compound 3-3 instead of raw compound 1-10. For compound 3, MS: 736.28 [M+H]⁺

### Example 110: Synthesis of Compound 4

### (2-((5-Bromo-2-((5-ethyl-2-methoxy-4-(4-(4-methylpiperazin-1-yl)piperidin-1-yl)phenyl)amino)pyrimidin-4-yl)amino)-5-(1-ethyl-1hydro-pyrazol-4-yl)phenyl)dimethylphosphine oxide

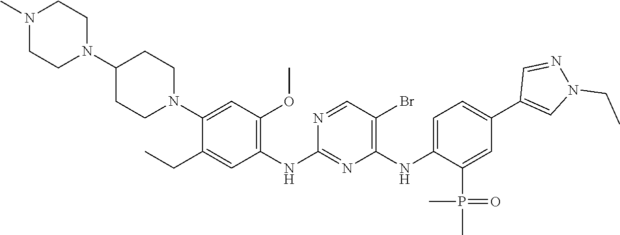 [SMILES: CCc1cc(Nc2ncc(Br)c(Nc3ccc(-c4cnn(CC)c4)cc3P(C)(C)=O)n2)c(OC)cc1N1CCC(N2CCN(C)CC2)CC1]

### Step 1: Synthesis of Compound 4-2

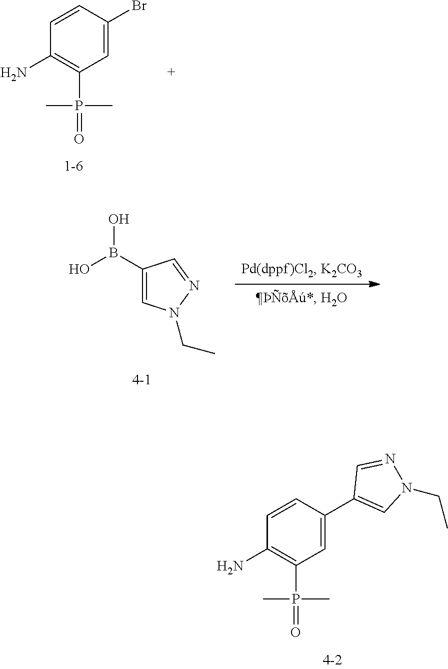

The compound 4-2 was synthesized as the method described for compound 1-8 using compound 4-1 instead of raw compound 1-7. For compound 4-2, MS: 264.12 [M+H]⁺

### Step 2: Synthesis of Compound 4-3

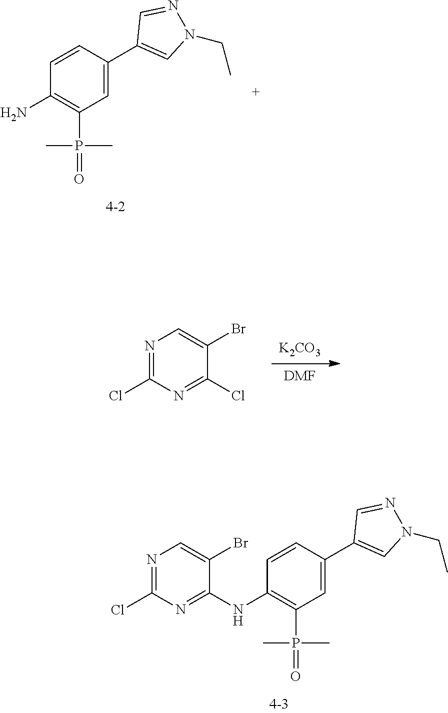

The compound 4-3 was synthesized as the method described for compound 1-10 using compound 4-2 instead of raw compound 1-8. For compound 4-3, MS: 454.01 [M+H]⁺

### Step 3: Synthesis of Compound 4

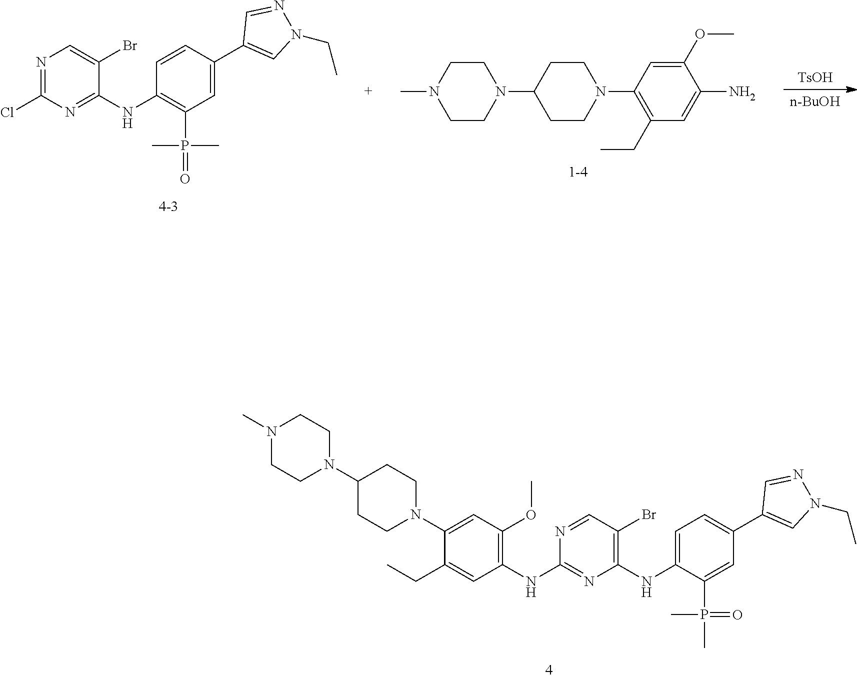

The compound 4 was synthesized as the method described for compound 1 using compound 4-3 instead of raw compound 1-10. For compound 4, MS: 750.29 [M+H]⁺

### Example 111: Synthesis of Compound 5

### 4′-((5-Bromo-2-((5-ethyl-2-methoxy-4-(4-(4-methylpiperazin-1-yl)piperidin-1-yl)phenyl)amino)pyrimidin-4-yl)amino)-3′-(dimethylphosphine oxide)-[1,1′-diphenyl]-3-carbonitrile

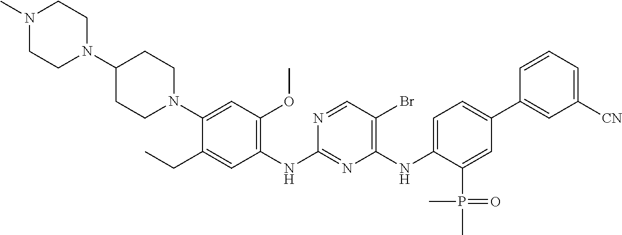 [SMILES: CCc1cc(Nc2ncc(Br)c(Nc3ccc(-c4cccc(C#N)c4)cc3P(C)(C)=O)n2)c(OC)cc1N1CCC(N2CCN(C)CC2)CC1]

### Step 1: Synthesis of Compound 5-2

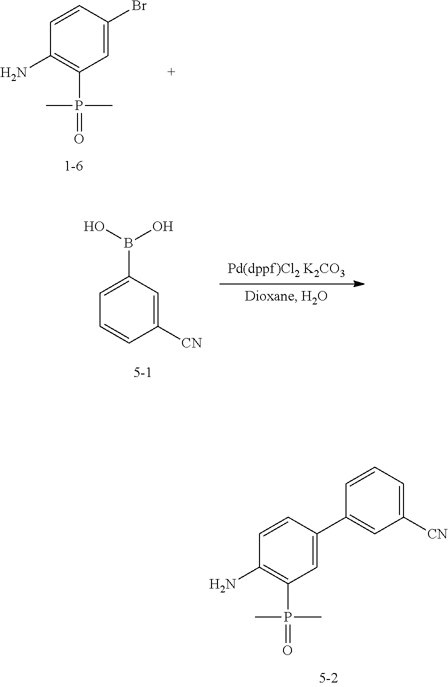 [SMILES: CP(C)(=O)c1cc(Br)ccc1N ; N#Cc1cccc(B(O)O)c1 ; "Dioxane, H2O" ; "Pd(dppf)Cl2 K2CO3" ; CP(C)(=O)c1cc(-c2cccc(C#N)c2)ccc1N]

The compound 5-2 was synthesized as the method described for compound 1-8 using compound 5-1 instead of raw compound 1-7. For compound 5-2, MS: 271.09 [M+H]⁺

### Step 2: Synthesis of Compound 5-3

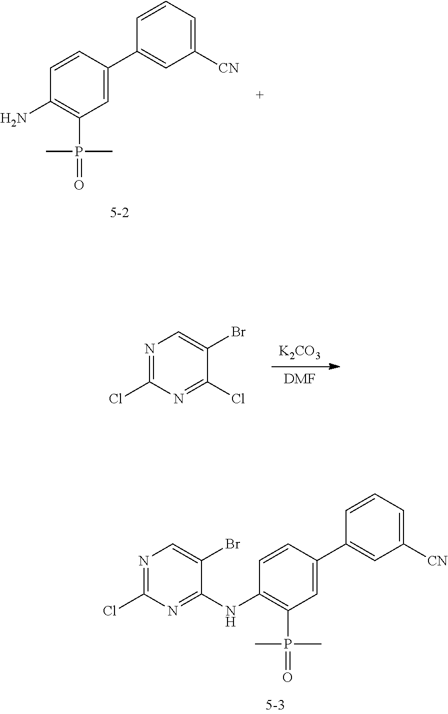

The compound 5-3 was synthesized as the method described for compound 1-10 using compound 5-2 instead of raw compound 1-8. For compound 5-3 MS: 460.99 [M+H]⁺

### Step 3: Synthesis of Compound 5

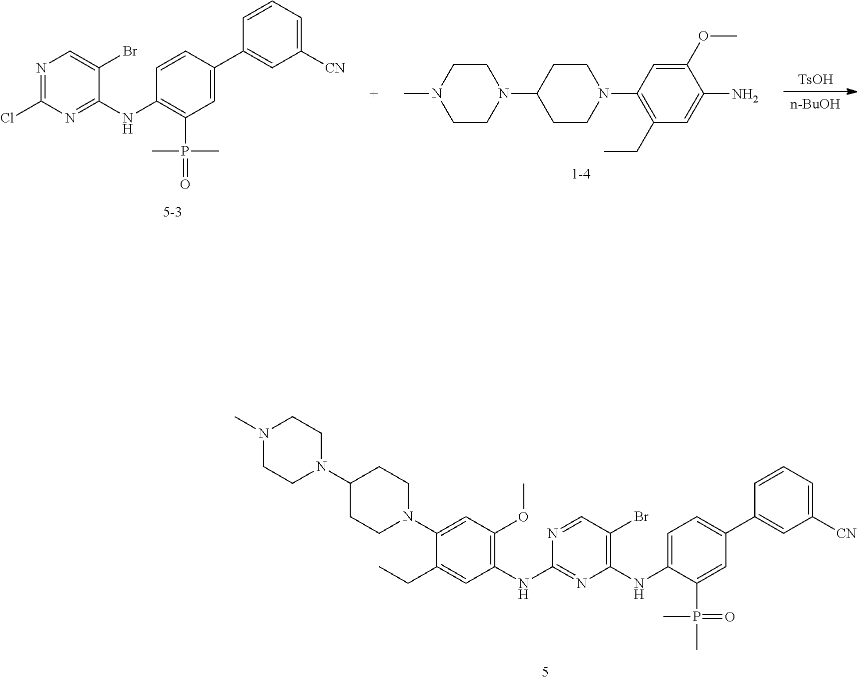

The compound 5 was synthesized as the method described for compound 1 using compound 5-3 instead of raw compound 1-10. For compound 5, MS: 757.27 [M+H]⁺

### Example 112: Synthesis of Compound 6

### (4-((5-Bromo-2-((5-ethyl-2-methoxy-4-(4-(4-methylpiperazin-1-yl)piperidin-1-yl)phenyl)amino)pyrimidin-4-yl)amino)-2′,4′-difluoro-[1,1′-biphenyl]-3-yl)dimethylphosphine oxide

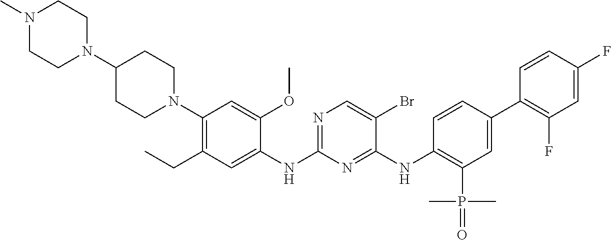 [SMILES: CCc1cc(Nc2ncc(Br)c(Nc3ccc(-c4ccc(F)cc4F)cc3P(C)(C)=O)n2)c(OC)cc1N1CCC(N2CCN(C)CC2)CC1]

### Step 1: Synthesis of Compound 6-2

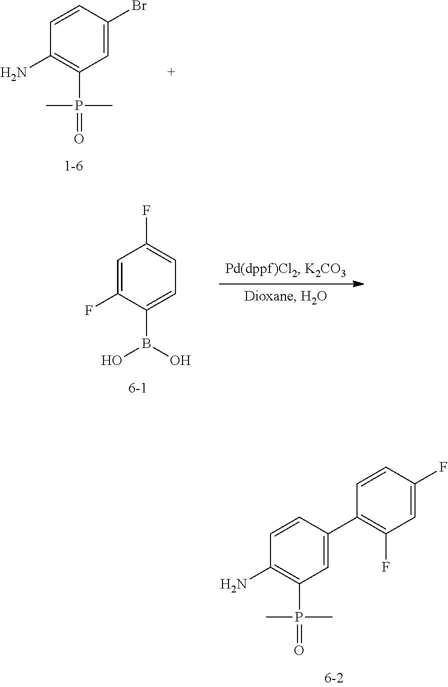 [SMILES: CP(C)(=O)c1cc(Br)ccc1N ; OB(O)c1ccc(F)cc1F ; "Dioxane, H2O" ; "Pd(dppf)Cl2, K2CO3" ; CP(C)(=O)c1cc(-c2ccc(F)cc2F)ccc1N]

Add compounds 1-6 (500 mg), 6-1 (384 mg), K₂CO₃ (836 mg), Pd(dppf)Cl₂ (147 mg), dioxane (10 mL) and water (2 mL) successively to the reaction flask, heat to 100° C. under nitrogen protection, and stir with heating for 12 hr. The reaction was complete as monitored by LCMS, and then stopped. Add water (50 mL) into the reaction solution. And then the reaction solution was extracted with dichloromethane (3×50 mL); the organic phase was washed with saturated brine (3×30 mL), dried over anhydrous sodium sulfate, separated and purified by column chromatography (dichloromethane:methanol=10:1), and the solvent was removed to obtain the brown solid of desired product compound 6-2 (500 mg), MS: 282.08 [M+H]⁺

### Step 2: Synthesis of Compound 6-3

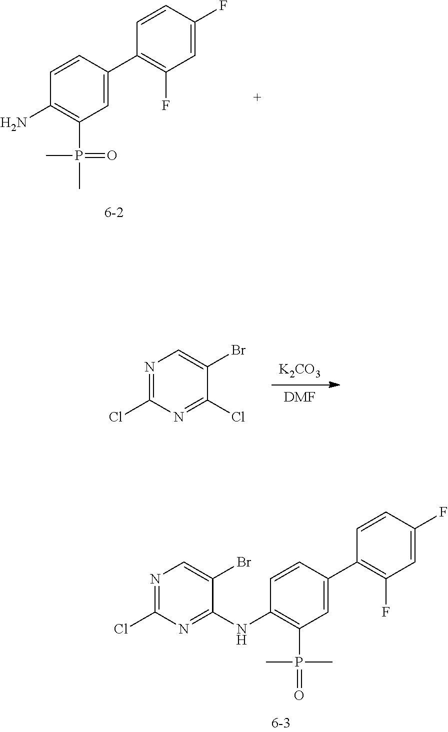

Add compounds 6-2 (500 mg), 3-Bromo-2,4-dichloropyrimidine (608 mg), K₂CO₃ (737 mg) and DMF (20 mL) successively to the reaction flask, heat to 100° C. and stir with heating for 12 hr. The reaction was complete as monitored by LCMS, and then stopped. The reaction solution was poured into water (50 mL), and extracted with ethyl acetate (3×50 mL); the organic phase was washed with saturated brine (3×30 mL), dried over anhydrous sodium sulfate, separated and purified by column chromatography (dichloromethane:methanol=12:1), and the solvent was removed to obtain the yellow solid of desired product compound 6-3 (500 mg), MS: 471.97 [M+H]⁺

### Step 3: Synthesis of Compound 6

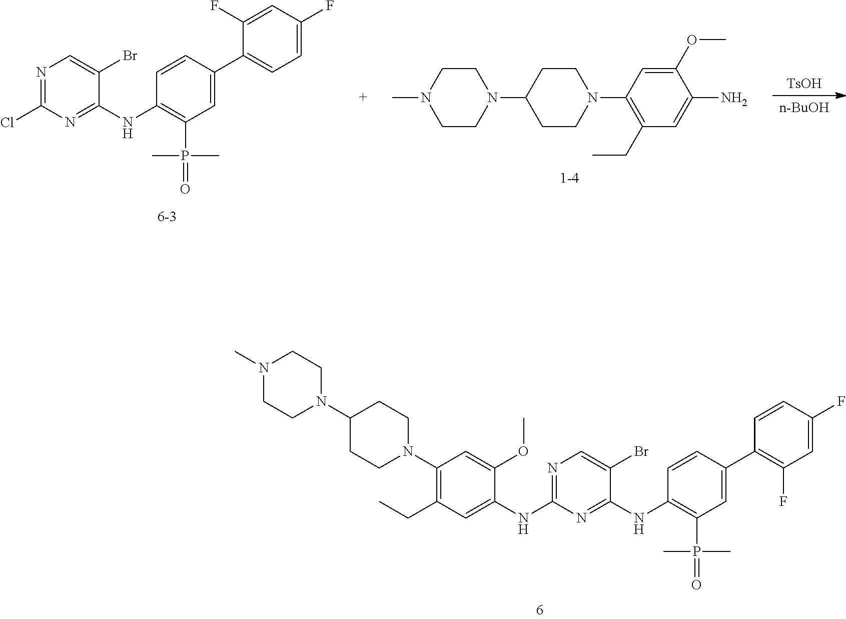

Add compound 6-3 (100 mg), 1-4 (70 mg), p-toluenesulfonic acid (55 mg) and n-butanol (2 mL) successively into the reaction flask, heat to 120° C., and stir for 12 hr. The reaction was complete as monitored by LCMS, and then stopped. The reaction solution was poured into sodium carbonate solution (5 mL), and extracted with dichloromethane (3×50 mL); the organic phase was washed with saturated brine (3×30 mL), dried over anhydrous sodium sulfate, separated and purified by column chromatography (dichloromethane:methanol=12:1), and then concentrated to obtain off-white solid of compound 6 (58 mg).

MS: 768.25 [M+H]⁺

¹H NMR (500 MHz, DMSO-d₆) δ 12.00 (s, 1H), 9.72 (s, 1H), 8.42-8.31 (m, 2H), 7.77-7.75 (m, 1H), 7.66-7.63 (m, 1H), 7.51-7.47 (m, 2H), 7.36 (s, 1H), 7.30-7.27 (m, 1H), 6.87 (s, 1H), 3.82-3.80 (m, 8H), 3.76-3.74 (m, 5H), 3.58-3.39 (m, 4H), 2.89-2.80 (m, 5H), 2.58-2.54 (m, 211), 2.22-2.21 (m, 2H), 1.96-1.94 (m, 2H), 1.87-1.84 (m, 6H).

### Example 113: Synthesis of Compound 7

### (2-((5-Chloro-2-((5-ethyl-2-methoxy-4-(4-(4-methylpiperazin-1-yl)piperidin-1-yl)phenyl)amino)pyrimidin-4-yl)amino)-5-(6,7-dihydro-5H-pyrazolo[5,1-b][1,3]nitrox-3-yl)phenyl)dimethylphosphine oxide

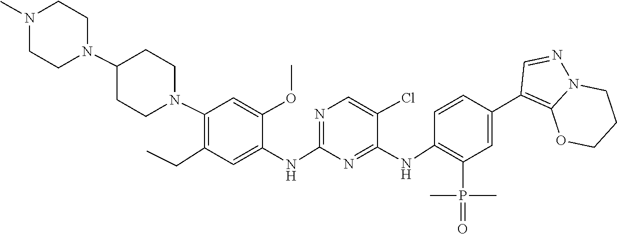

### Step 1: Synthesis of Compound 7

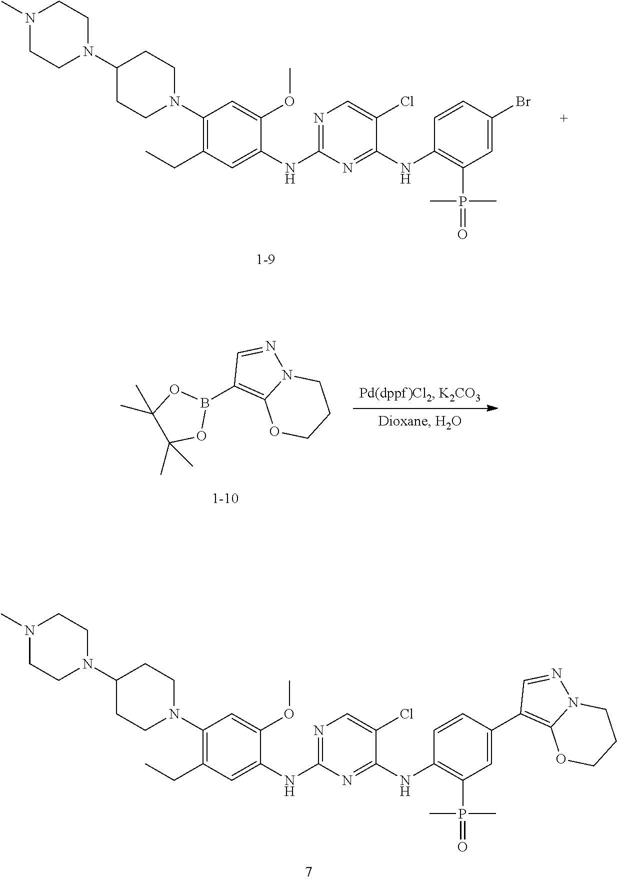 [SMILES: CCc1cc(Nc2ncc(Cl)c(Nc3ccc(Br)cc3P(C)(C)=O)n2)c(OC)cc1N1CCC(N2CCN(C)CC2)CC1 ; CC1(C)OB(c2cnn3c2OCCC3)OC1(C)C ; "Dioxane, H2O" ; "Pd(dppf)Cl2, K2CO3" ; CCc1cc(Nc2ncc(Cl)c(Nc3ccc(-c4cnn5c4OCCC5)cc3P(C)(C)=O)n2)c(OC)cc1N1CCC(N2CCN(C)CC2)CC1]

Add 5-Bromo-2,4-dichloropyrimidine (100 mg), compound 1-10 (36 mg), K₂CO₃ (60 mg), Pd(dppf)Cl₂ (11 mg), dioxane (5 mL) and water (1 mL) successively to the reaction flask, heat to 100° C. under nitrogen protection, and stir with heating for 12 hr. The reaction was complete as monitored by LCMS, and then stopped. Add water (10 mL) to the reaction solution; and then the reaction solution was extracted with dichloromethane (3×10 mL); the organic phase was washed with saturated brine (3×10 mL), dried over anhydrous sodium sulfate, separated and purified by column chromatography (dichloromethane:methanol=10:1), and the solvent was removed to obtain the brown solid of desired product compound 7 (50 mg), MS: 734.34 [M+H]⁺

### Example 114: Synthesis of Compound 8

### (2-((5-Bromo-2-((5-ethyl-2-methoxy-4-(4(4-methylpiperazin-1-yl)piperidin-1-yl)phenyl)amino)pyrimidin-4-yl)amino)-5-(2-methyl-2H-1,2,3-triazol-4-yl)phenyl)dimethylphosphine oxide

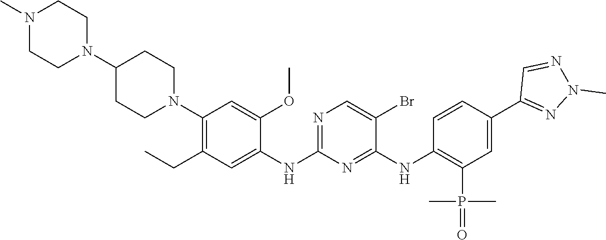

### Step 1: Synthesis of Compound 8-1

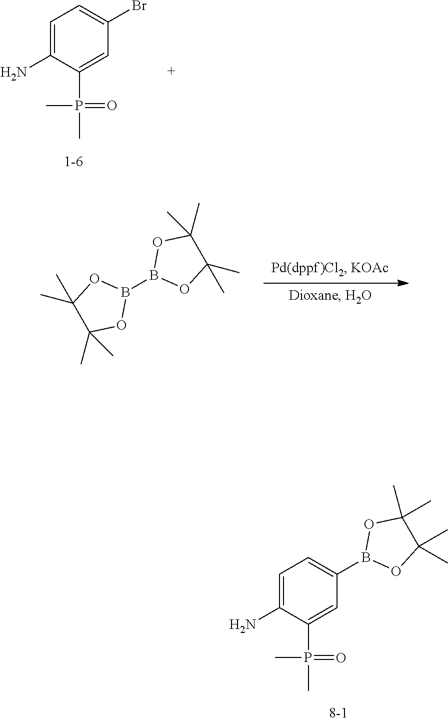

Add compound 1-6 (5.0 g), bis(pinacolato)diborane (7.68 g), Pd(dppf)Cl₂ (1.65 g), potassium acetate (4.95 g) and dioxane (50 mL) successively to the reaction flask, heat to 100° C. under nitrogen protection and stir with heating for 3 hr. The reaction was complete as monitored by LCMS, and then stopped. The reaction solution was cooled down to room temperature, filtered and concentrated to obtain the crude product. The crude product was separated and purified by column chromatography (dichloromethane:methanol=15:1). The solvent was removed to get the brown solid of desired product 8-1 (2.0 g), MS: 296.15 [M+H]⁺

### Step 2: Synthesis of Compound 8-3

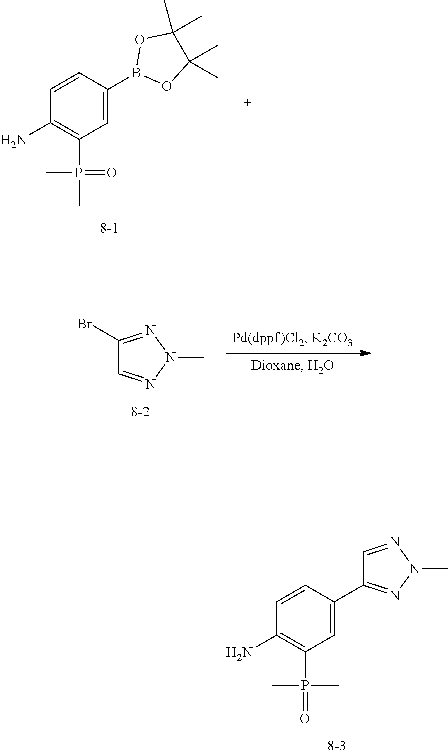

Add compounds 8-1 (4.0 g), 8-2 (2.20 g), K₂CO₃ (5.62 g), Pd(dppf)Cl₂ (992 mg), dioxane (80 mL) and water (20 mL) to the reaction flask, heat to 100° C. under nitrogen protection and stir with heating for 12 hr. The reaction was complete as monitored by LCMS, and then stopped. The reaction solution was poured into water (50 mL), and extracted with dichloromethane (3×100 mL); the organic phase was washed with saturated brine (3×50 mL), dried over anhydrous sodium sulfate, separated and purified by column chromatography (dichloromethane:methanol=10:1), and the solvent was removed to obtain brown solid of desired product 8-3 (3 g), MS: 251.10 [M+H]⁺

### Step 2: Synthesis of Compound 8-4

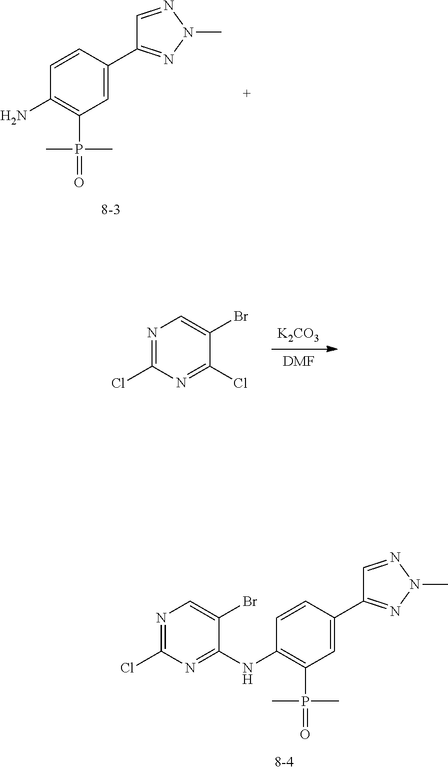

The compound 8-4 was synthesized as the method described for compound 1-10 using compound 8-3 instead of raw compound 1-8. For compound 8-4, MS: 440.99 [M+H]⁺

### Step 3: Synthesis of Compound 8

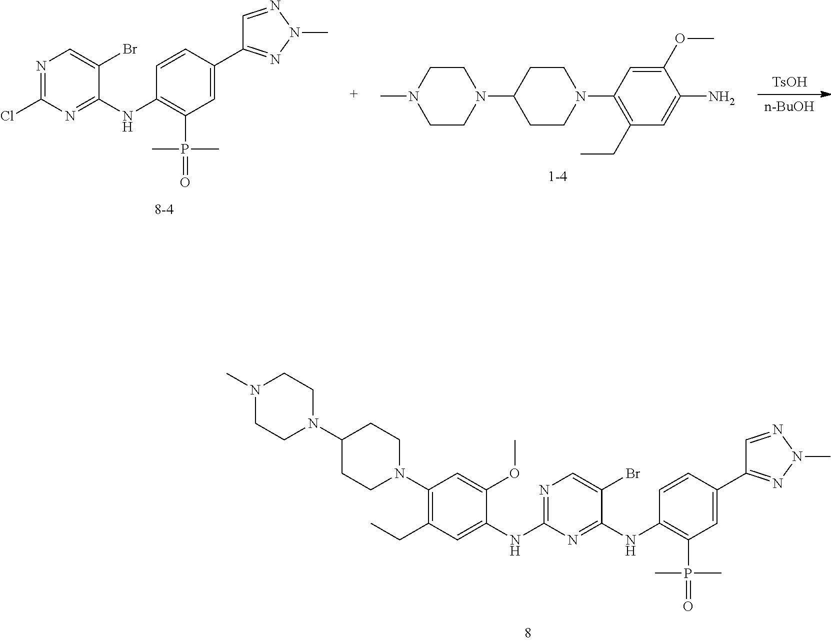

The compound 8 was synthesized as the method described for compound 1 using compound 8-4 instead of raw compound 1-10. For compound 8, MS: 737.27 [M+H]⁺

### Example 115: Synthesis of Compound 9

### (2-((5-Bromo-2-((5-ethyl-2-methoxy-4-(4-(4-methylpiperazin-1-yl)piperidin-1-yl)phenyl)amino)pyrimidin-4-yl)amino)-5-(1-methyl-1H-pyrazol[3,4-b]pyridin-3-yl)phenyl)dimethylphosphine oxide

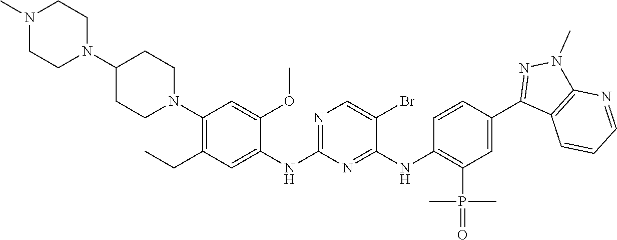 [SMILES: CCc1cc(Nc2ncc(Br)c(Nc3ccc(-c4nn(C)c5ncccc45)cc3P(C)(C)=O)n2)c(OC)cc1N1CCC(N2CCN(C)CC2)CC1]

### Step 1: Synthesis of Compound 9-2

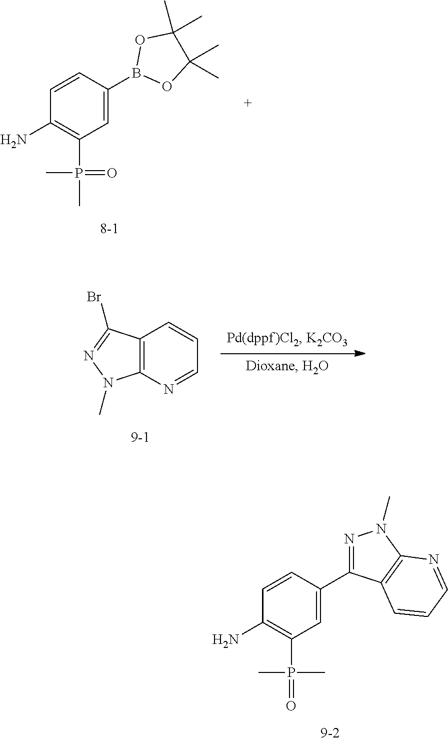 [SMILES: CC1(C)OB(c2ccc(N)c(P(C)(C)=O)c2)OC1(C)C ; Cn1nc(Br)c2cccnc21 ; "Dioxane, H2O" ; "Pd(dppf)Cl2, K2CO3" ; Cn1nc(-c2ccc(N)c(P(C)(C)=O)c2)c2cccnc21]

The compound 9-2 was synthesized as the method described for compound 8-3 using compound 9-2 instead of raw compound 8-2. For compound 9-1, MS: 301.11 [M+H]⁺

### Step 2: Synthesis of Compound 9-3

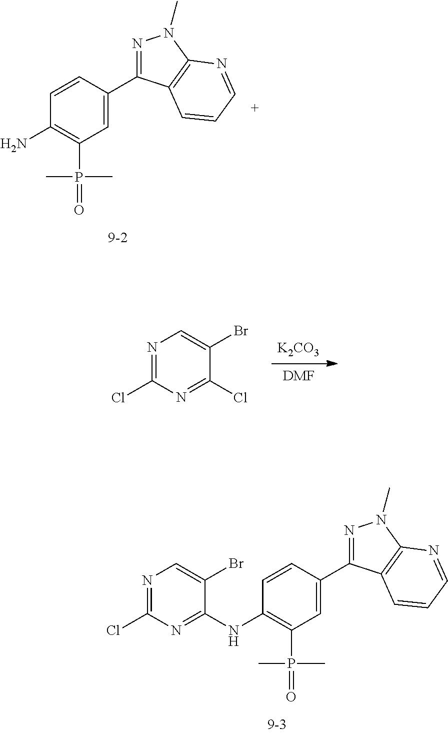

The compound 9-3 was synthesized as the method described for compound 1-10 using compound 9-2 instead of raw compound 1-. For compound 9-3, MS: 491.01 [M+H]⁺

### Step 3: Synthesis of Compound 9

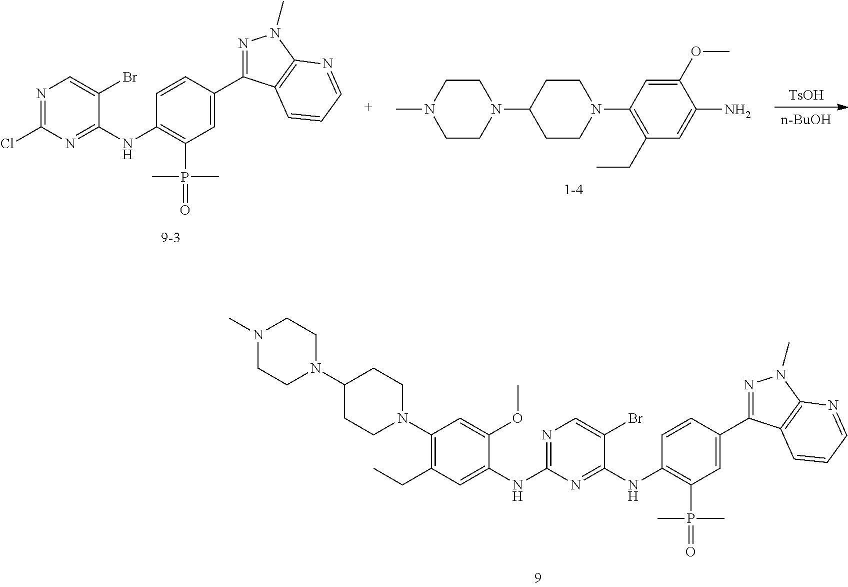

The compound 9 was synthesized as the method described for compound 1 using compound 9-3 instead of raw compound 1-10.

For compound 9, MS: 787.29 [M+H]⁺

¹H NMR (500 MHz, DMSO-d₆) δ 11.53 (s, 1H), 8.68-8.67 (m, 1H), 8.57-8.55 (m, 1H), 8.34-8.33 (m, 2H), 8.09-8.06 (m, 1H), 7.90 (s, 1H), 7.48-7.38 (m, 2H), 7.12-7.11 (m, 1H), 6.84 (s, 1H), 4.14 (s, 3H), 3.79-3.73 (m, 5H), 3.57-3.52 (m, 4H), 3.50-3.49 (m, 411), 3.16-3.12 (m, 511), 2.86-2.77 (m, 511), 2.14 (s, 1H), 2.10-2.05 (m, 2H), 1.91-88 (m, 8H).

### Example 116: Synthesis of Compound 10

### 4′-((5-Bromo-2-((5-ethyl-2-methoxy-4-(4-(4-methylpiperazin-1-yl)piperidin-1-yl)phenyl)amino)pyrimidin-4-yl)amino)-3′-(dimethylphosphino)-[1,1′-biphenyl]-4-carbonitrile

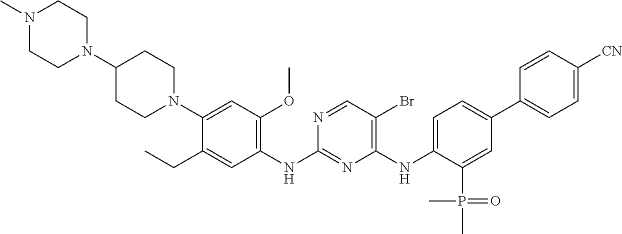 [SMILES: CCc1cc(Nc2ncc(Br)c(Nc3ccc(-c4ccc(C#N)cc4)cc3P(C)(C)=O)n2)c(OC)cc1N1CCC(N2CCN(C)CC2)CC1]

### Step 1: Synthesis of Compound 10-2

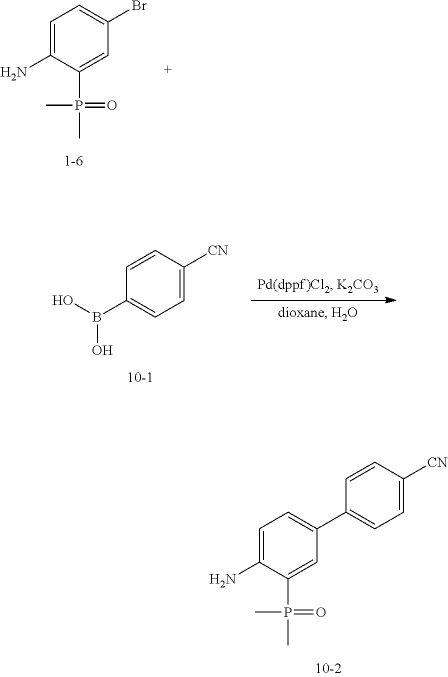 [SMILES: CP(C)(=O)c1cc(Br)ccc1N ; N#Cc1ccc(B(O)O)cc1 ; "Pd(dppf)Cl2, K2CO3" ; "dioxane, H2O" ; CP(C)(=O)c1cc(-c2ccc(C#N)cc2)ccc1N]

The compound 10-2 was synthesized as the method described for compound 1-8 using compound 10-1 instead of raw compound 1-7. For compound 10-2, MS: 271.09 [M+H]⁺

### Step 2: Synthesis of Compound 10-3

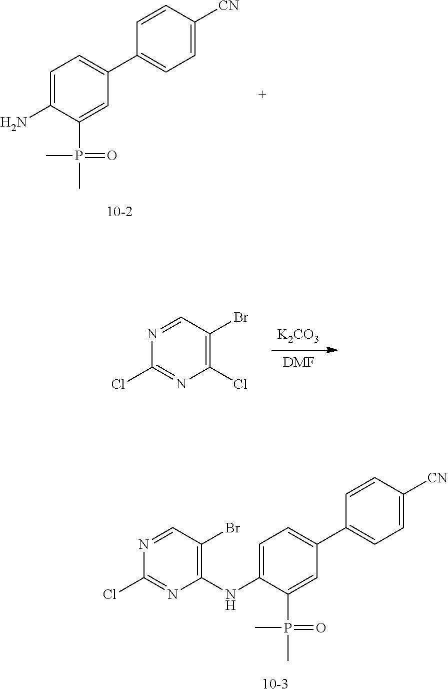

The compound 10-3 was synthesized as the method described for compound 1-10 using compound 10-2 instead of raw compound 1-8. For compound 10-3, MS: 460.99 [M+H]⁺

### Step 3: Synthesis of Compound 10

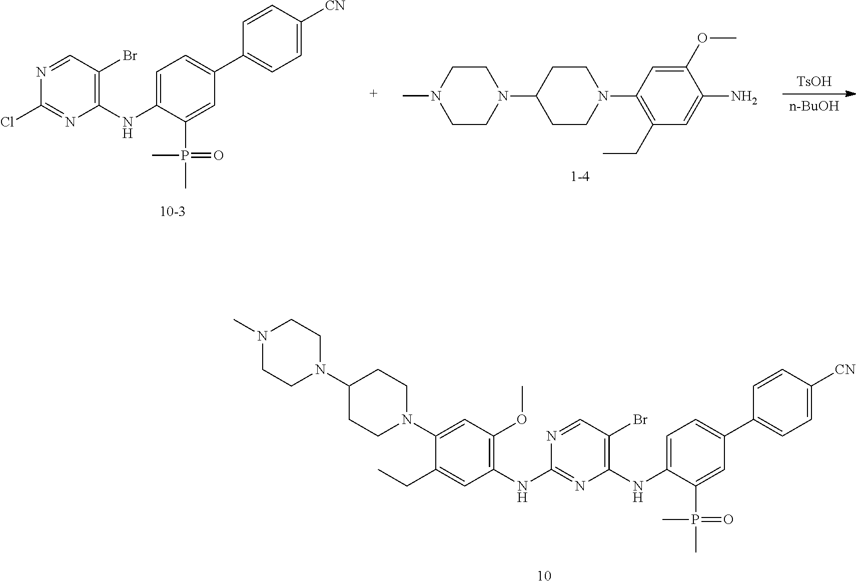

The compound 10 was synthesized as the method described for compound 1 using compound 10-3 instead of raw compound 1-10.

For compound 10, MS: 757.27 [M+H]⁺

¹H NMR (500 MHz, DMSO-d₆) δ 11.14 (s, 1H), 8.38 (s, 1H), 8.25 (d, 2H), 8.20 (s, 1H), 8.10-7.92 (m, 4H), 7.87-7.40 (m, 2H), 7.63 (s, 1H), 7.38 (s, 1H), 6.81 (s, 1H), 3.76 (s, 3H), 2.96 (d, 2H), 2.70-2.51 (m, 10H), 2.49-2.38 (m, 2H), 2.37-2.22 (m, 2H), 1.89 (s, 3H), 1.86 (s, 3H), 1.77-1.71 (m, 2H), 1.59-1.42 (m, 2H), 1.01 (t, 3H).

### Example 117: Synthesis of Compound 11

### (2-((5-Bromo-2-((5-ethyl-2-methoxy-4-(4-(4-methylpiperazin-1-yl)piperidin-1-yl)phenyl)amino)pyrimidin-4-yl)amino)-5-(1,3-dihydroisobenzofuran-4-yl)phenyl)dimethylphosphine oxide

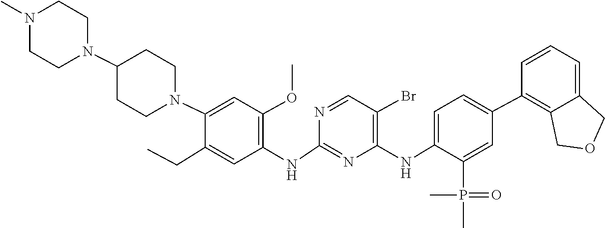 [SMILES: CCc1cc(Nc2ncc(Br)c(Nc3ccc(-c4cccc5c4COC5)cc3P(C)(C)=O)n2)c(OC)cc1N1CCC(N2CCN(C)CC2)CC1]

### Step 1: Synthesis of Compound 11-2

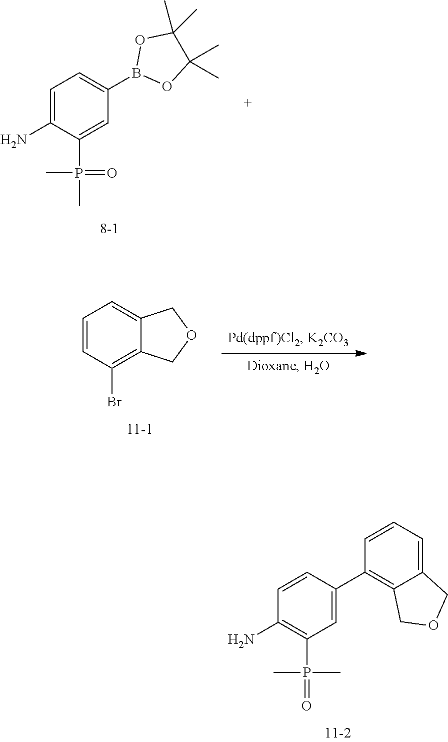 [SMILES: CC1(C)OB(c2ccc(N)c(P(C)(C)=O)c2)OC1(C)C ; Brc1cccc2c1COC2 ; "Dioxane, H2O" ; "Pd(dppf)Cl2, K2CO3" ; CP(C)(=O)c1cc(-c2cccc3c2COC3)ccc1N]

The compound 11-2 was synthesized as the method described for compound 8-3 using compound 11-1 instead of raw compound 18-2. For compound 11-2, MS: 288.11 [M+H]⁺

### Step 3: Synthesis of Compound 11-3

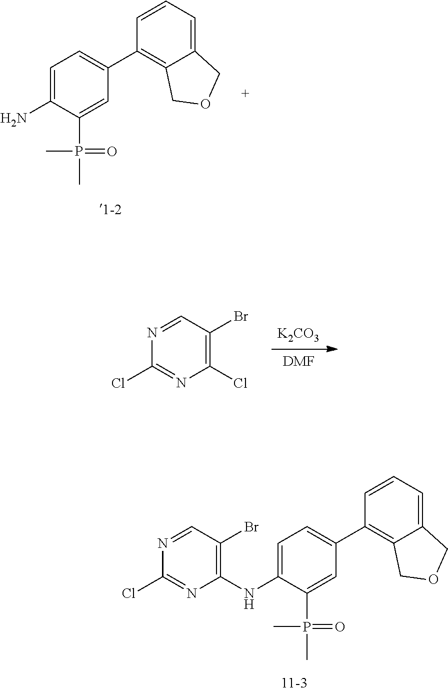

The compound 11-3 was synthesized as the method described for compound 1-10 using compound 11-2 instead of raw compound 1-8. For compound 11-3, MS: 478.00 [M+H]⁺

### Step 4: Synthesis of Compound 11

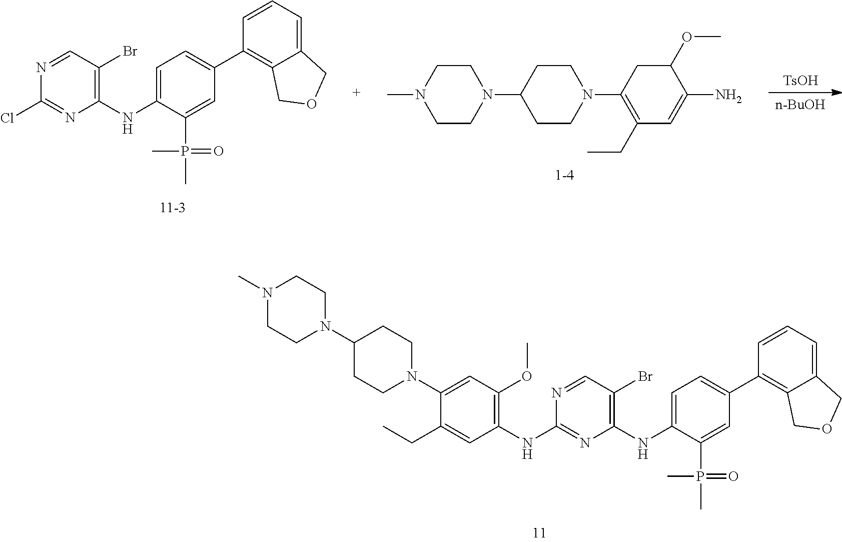

The compound 11 was synthesized as the method described for compound 1 using compound 11-3 instead of raw compound 1-10.

For compound 11, MS: 774.28 [M+H]⁺.

¹H NMR (500 MHz, DMSO-d₆) δ 11.10 (s, 1H), 8.37 (s, 1H), 8.25 (s, 1H), 8.18 (s, 1H), 7.55 (d, 1H), 7.42 (d, 11H), 7.39-7.30 (m, 4H), 6.78 (s, 1H), 5.14 (s, 2H), 5.08 (s, 2H), 3.74 (s, 3H), 2.99 (d, 3H), 2.89-2.52 (m, 7H), 2.49-2.22 (m, 6H), 1.83 (s, 3H), 1.80 (s, 3H), 1.749-1.74 (m, 3H), 1.65-1.50 (m, 3H), 1.03 (t, 3H).

### Example 118: Synthesis of Compound 12

### (2-((5-Bromo-2-((5-ethyl-2-methoxy-4-(4-(4-methylpiperazin-1-yl)piperidin-1-yl)phenyl)amino)pyrimidin-4-yl)amino)-5-(thiazol-2-yl)phenyl)dimethylphosphine oxide

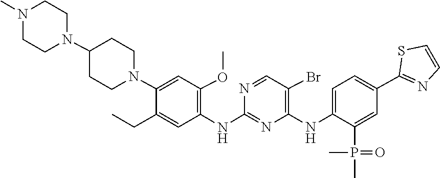 [SMILES: CCc1cc(Nc2ncc(Br)c(Nc3ccc(-c4nccs4)cc3P(C)(C)=O)n2)c(OC)cc1N1CCC(N2CCN(C)CC2)CC1]

### Step 1: Synthesis of Compound 12-2

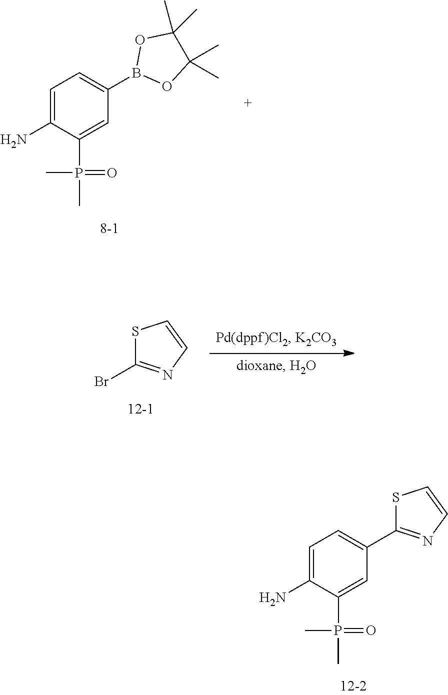 [SMILES: CC1(C)OB(c2ccc(N)c(P(C)(C)=O)c2)OC1(C)C ; Brc1nccs1 ; "Pd(dppf)Cl2, K2CO3" ; "dioxane, H2O" ; CP(C)(=O)c1cc(-c2nccs2)ccc1N]

The compound 12-2 was synthesized as the method described for compound 8-1 using compound 12-1 instead of raw compound 8-2. For compound 12-2, MS: 253.05 [M+H]⁺

### Step 2: Synthesis of Compound 12-3

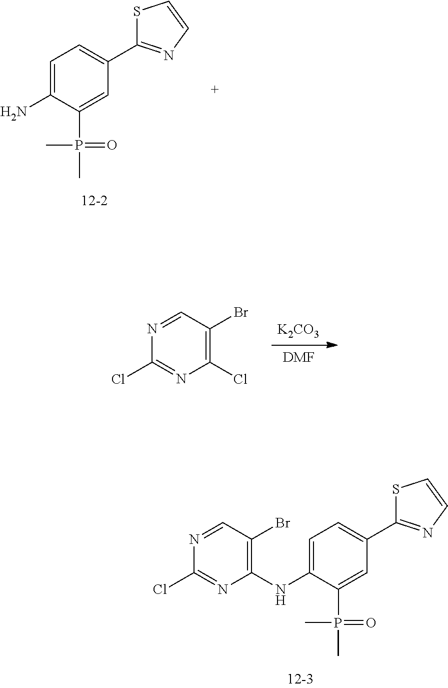

The compound 12-3 was synthesized as the method described for compound 1-10 using compound 12-2 instead of raw compound 1-8. For compound 12-3, MS: 442.94 [M+H]⁺

### Step 3: Synthesis of Compound 12

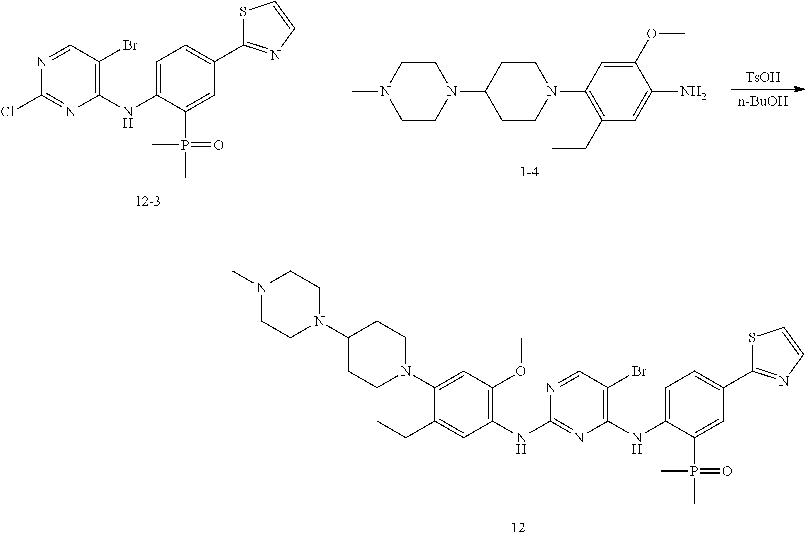

The compound 12 was synthesized as the method described for compound 1 using compound 12-3 instead of raw compound 1-10. For compound 12, MS: 739.22 [M+H]⁺

### Example 119: Synthesis of Compound 13

### (2-((5-Bromo-2-((5-ethyl-2-methoxy-4-(4-(4-methyl-1-yl)piperazin-1-yl)piperidin-1-yl)phenyl)amino)pyrimidin-4-yl)amino)-5-(6-methoxypyrazin-2-yl)phenyl)dimethylphosphine oxide

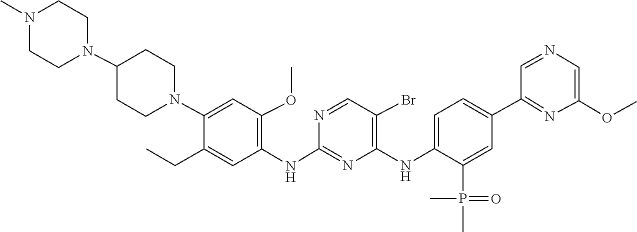

### Step 1: Synthesis of Compound 13-2

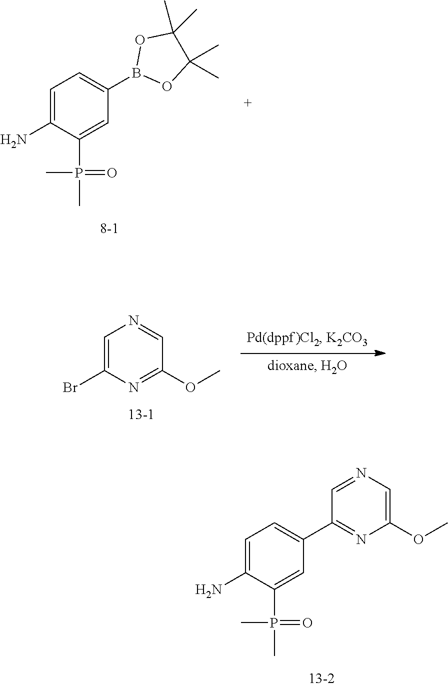

The compound 13-2 was synthesized as the method described for compound 8-3 using compound 13-1 instead of raw compound 8-2. For compound 13-2, MS: 278.10 [M+H]⁺

### Step 2: Synthesis of Compound 13-3

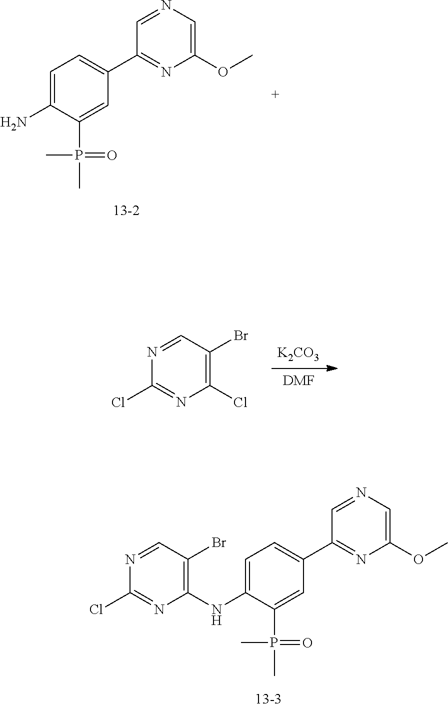

The compound 13-3 was synthesized as the method described for compound 1-10 using compound 13-2 instead of raw compound 1-8. For compound 13-3, MS: 467.99 [M+H]⁺

### Step 3: Synthesis of Compound 13

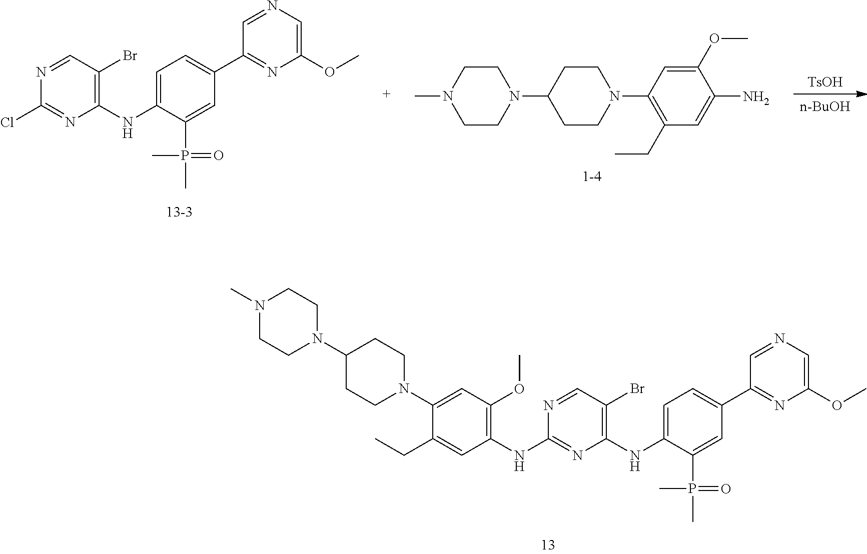

The compound 13 was synthesized as the method described for compound 1 using compound 13-3 instead of raw compound 1-10. For compound 13, MS: 764.27 [M+H]⁺

### Example 120: Synthesis of Compound 14

### (2-((5-Chloro-2-((5-ethyl-2-methoxy-4-(4-(4-methylpiperazin-1-yl)piperidin-1-yl)phenyl)amino)pyrimidin-4-yl)amino)-5-(thiazol-4-yl)phenyl)dimethylphosphine oxide

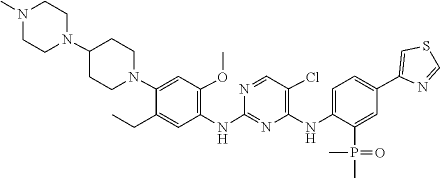 [SMILES: CCc1cc(Nc2ncc(Cl)c(Nc3ccc(-c4cscn4)cc3P(C)(C)=O)n2)c(OC)cc1N1CCC(N2CCN(C)CC2)CC1]

### Step 1: Synthesis of Compound 14-1

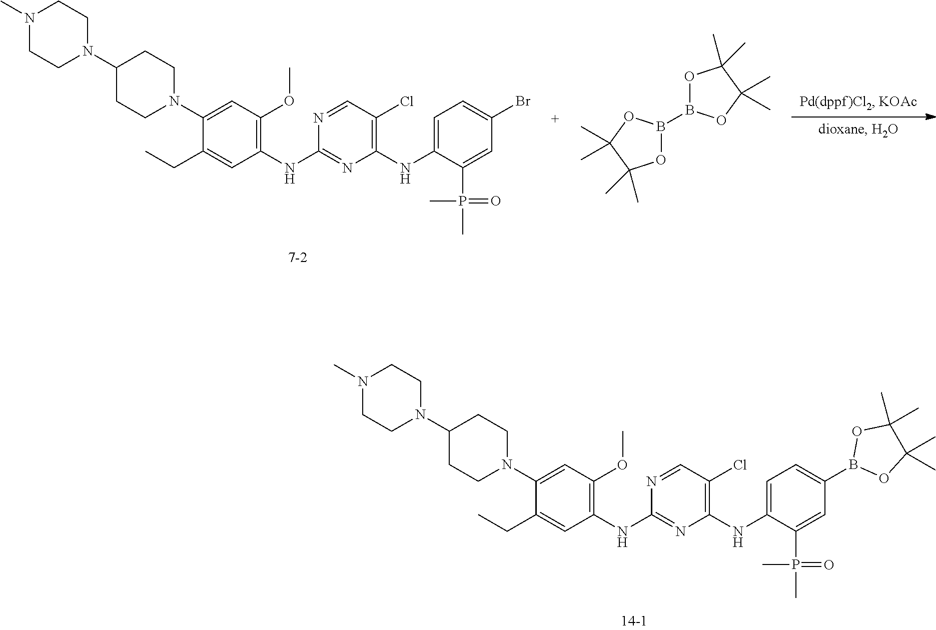

Add compound 7-2 (1.5 g), bis(pinacolato)diborane (0.77 g), Pd(dppf)Cl₂ (0.18 g), potassium acetate (0.64 g) and dioxane (20 ml) to the reaction flask, heat to 100° C. under nitrogen protection and stir with heating for 3 hr. The reaction was complete as monitored by LCMS, and then stopped. The reaction solution was cooled down to room temperature, and filtered. The crude product was obtained after removing the solvent, separated and purified by column chromatography (dichloromethane:methanol=15:1). The solvent was removed to get the yellow solid of desired product 14-1 (0.8 g), MS: 738.38 [M+H]⁺

### Step 2: Synthesis of Compound 14

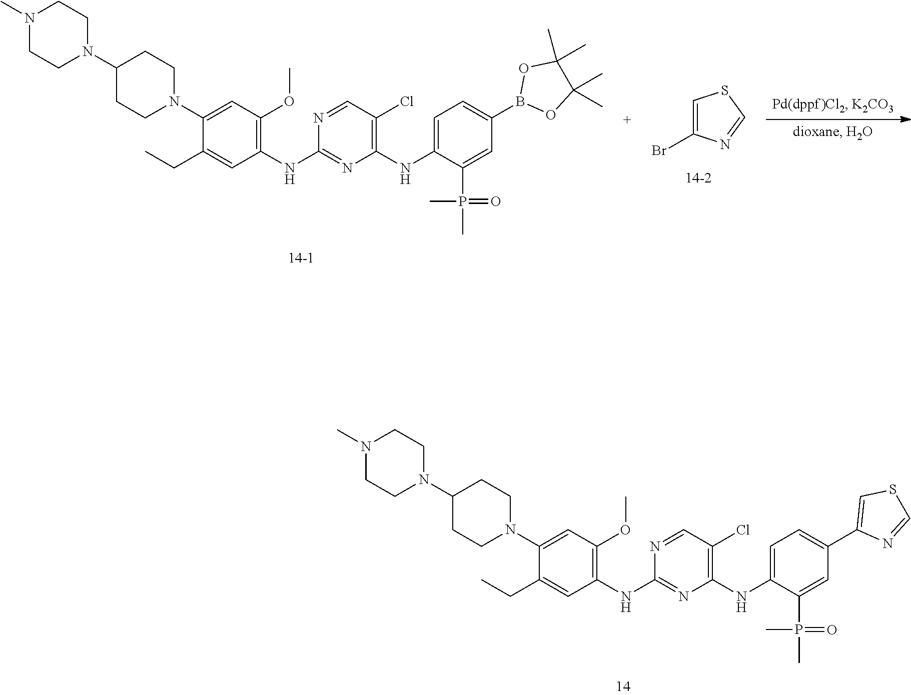

Add compounds 14-1 (100 mg), 14-2 (22 mg), K₂CO₃ (20 g), Pd(dppf)Cl₂ (14 mg), dioxane (2 mL) and water (0.5 mL) successively to the reaction flask, heat to 100° C. under nitrogen protection and stir with heating for 12 hr. The reaction was complete as monitored by LCMS, and then stopped. Add water (15 mL) into the reaction solution. And then the reaction solution was extracted with dichloromethane (3×15 mL); the organic phase was washed with saturated brine (3×30 mL), and dried over anhydrous sodium sulfate. The solvent was spin dry. The crude product was separated and purified by column chromatography on silica gel (dichloromethane:methanol=10:1), and the solvent was removed to obtain the yellow solid of desired compound 14 (29 mg).

For compound 14, MS: 695.27 [M+H]⁺.

¹H NMR (500 MHz, DMSO-d6) δ 11.28 (s, 1H), 9.20 (s, 1H), 8.44 (s, 1H), 8.21 (d, 2H), 8.11 (s, 1H), 8.03 (d, 1H), 7.93 (d, 1H), 7.38 (s, 1H), 6.80 (s, 1H), 3.75 (s, 3H), 3.04 (d, 2H), 2.69-2.53 (m, 10H), 2.49-2.23 (m, 6H), 1.86 (s, 3H), 1.83 (s, 3H), 1.82-1.79 (m, 2H), 1.56-1.52 (m, 2H), 1.00 (t, 3H).

### Example 121: Synthesis of Compound 15

### (2-((5-Chloro-2-((5-ethyl-2-methoxy-4-(4-(4-methylpiperazin-1-yl)piperidin-1-yl)phenyl)amino)pyrimidin-4-yl)amino)-5-(thiazol-5-yl)phenyl)dimethylphosphine oxide

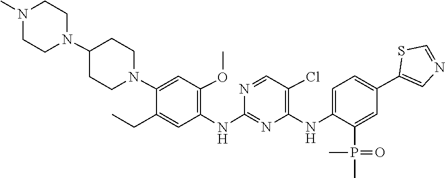 [SMILES: CCc1cc(Nc2ncc(Cl)c(Nc3ccc(-c4cncs4)cc3P(C)(C)=O)n2)c(OC)cc1N1CCC(N2CCN(C)CC2)CC1]

### Step 1: Synthesis of Compound 15

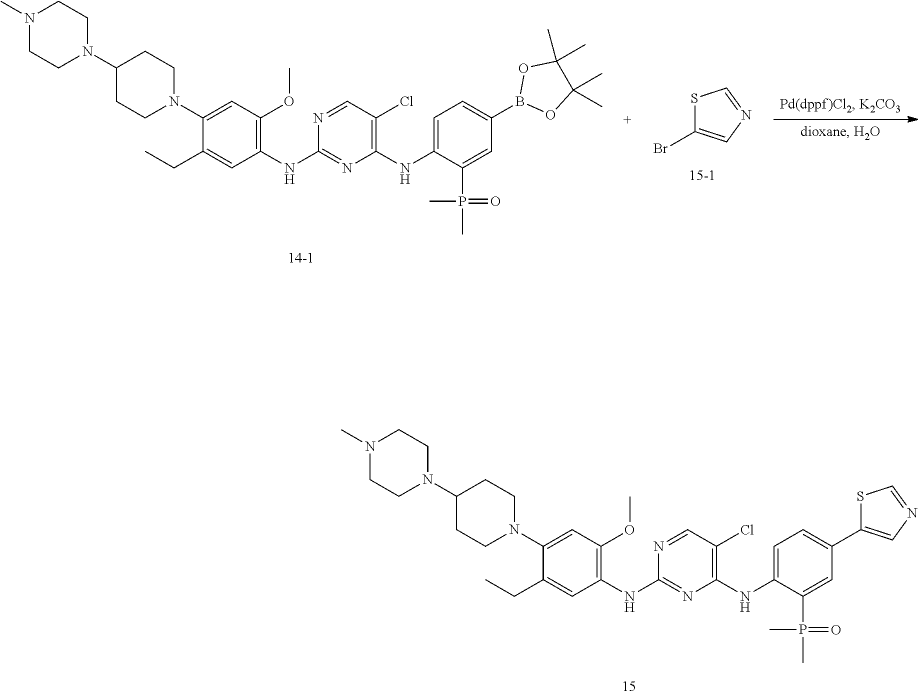

The compound 15 was synthesized as the method described for compound 14 using compound 15-1 instead of raw compound 14-2. For compound 15, MS: 695.27 [M+H]⁺

### Example 122: Synthesis of Compound 16

### (2-((5-Bromo-2-((5-ethyl-2-methoxy-4-(4-(4-methylpiperazin-1-yl)piperidin-1-yl)phenyl)amino)pyrimidin-4-yl)amino)-5-(furan-2-yl)phenyl)dimethylphosphine oxide

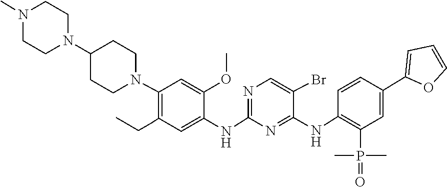 [SMILES: CCCc1cc(Nc2ncc(Br)c(Nc3ccc(-c4ccco4)cc3P(C)(C)=O)n2)c(OC)cc1N1CCC(N2CCN(C)CC2)CC1]

### Step 1: Synthesis of Compound 16-2

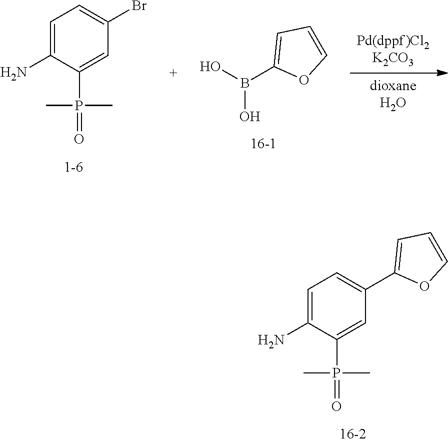 [SMILES: CP(C)(=O)c1cc(Br)ccc1N ; OB(O)c1ccco1 ; "Pd(dppf)Cl2K2CO3" ; "dioxaneH2O" ; CP(C)(=O)c1cc(-c2ccco2)ccc1N]

The compound 16-2 was synthesized as the method described for compound 1-8 using compound 16-1 instead of raw compound 1-7. For compound 16-2, MS: 236.08 [M+H]⁺

### Step 2: Synthesis of Compound 16-3

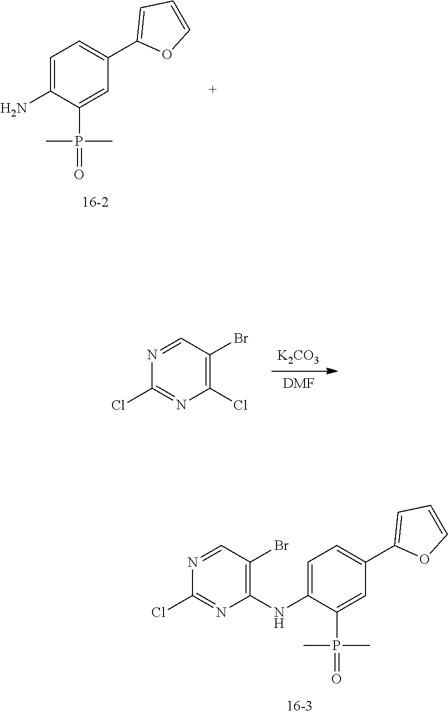

The compound 16-3 was synthesized as the method described for compound 1-10 using compound 16-2 instead of raw compound 1-8. For compound 16-3, MS: 425.97 [M+H]⁺

### Step 3: Synthesis of Compound 16

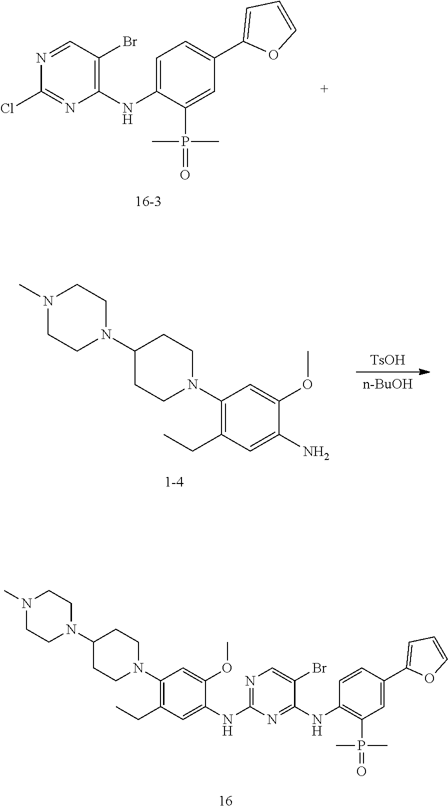

The compound 16 was synthesized as the method described for compound 1 using compound 16-3 instead of raw compound 1-10. For compound 16, MS: 722.25 [M+H]⁺

### Example 123: Synthesis of Compound 17

### (5-(Benzo[d]oxazol-5-yl)-2-((5-chloro-2-((5-ethyl-2-methoxy-4-(4-(4-methylpiperazin-1-yl)piperidin-1-yl)phenyl)amino)pyrimidin-4-yl)amino)phenyl)dimethylphosphine oxide

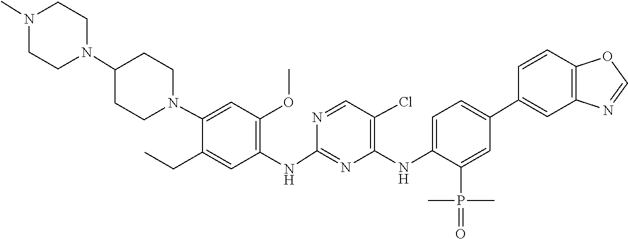 [SMILES: CCc1cc(Nc2ncc(Cl)c(Nc3ccc(-c4ccc5ocnc5c4)cc3P(C)(C)=O)n2)c(OC)cc1N1CCC(N2CCN(C)CC2)CC1]

### Step 2: Synthesis of Compound 17

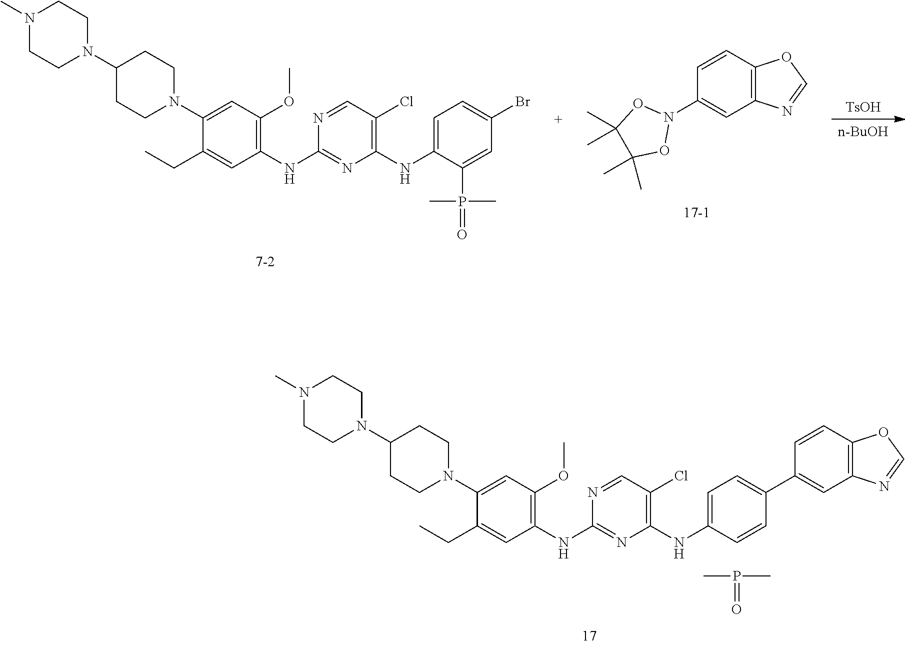

Dissolve 7-2 (100 mg) and 17-1 (56 mg) to 1120 (2 mL) and dioxane (8 mL) in a reactor equipped with a stir bar and nitrogen protector, add Pd(dppf)Cl₂ (23 mg) and K₂CO₃ (30 mg) at room temperature, and the gas in reactor was replaced with nitrogen for three times. After well stirring, the solution was transferred to an oil bath, heated to 110° C., and stirred at 110° C. for 4 hr. The reaction was complete as monitored by LCMS. The reaction solution was cooled down to room temperature, poured into water, and extracted with EtOAc (3×30 mL); the organic phase was dried over anhydrous sodium sulfate, and concentrated by rotary evaporator. Silica gel was added to the concentrated organic phase to mix the sample, and then which was separated and purified by PE:EtOAc column with gradient from 0% to 100%, and by MeOH:H₂O (0% to 40%). After that, it was concentrated and dried to obtain the compound 17 (17 mg), MS: 729.31 [M+H]⁺

### Example 124: Synthesis of Compound 18

### (2-((5-Chloro-2-((5-ethyl-2-methoxy-4-(4-(4-methylpiperazin-1-yl)piperidin-1-yl)phenyl)amino)pyrimidin-4-yl)amino)-5-(5-fluoropyrimidin-2-yl)phenyl)dimethylphosphine oxide

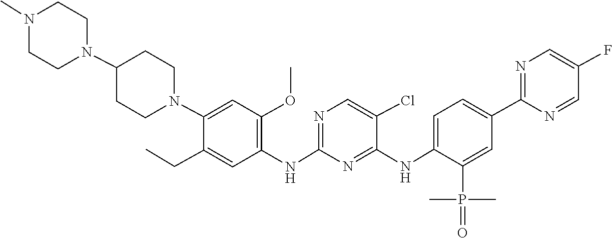 [SMILES: CCc1cc(Nc2ncc(Cl)c(Nc3ccc(-c4ncc(F)cn4)cc3P(C)(C)=O)n2)c(OC)cc1N1CCC(N2CCN(C)CC2)CC1]

### Step 1: Synthesis of Compound 18

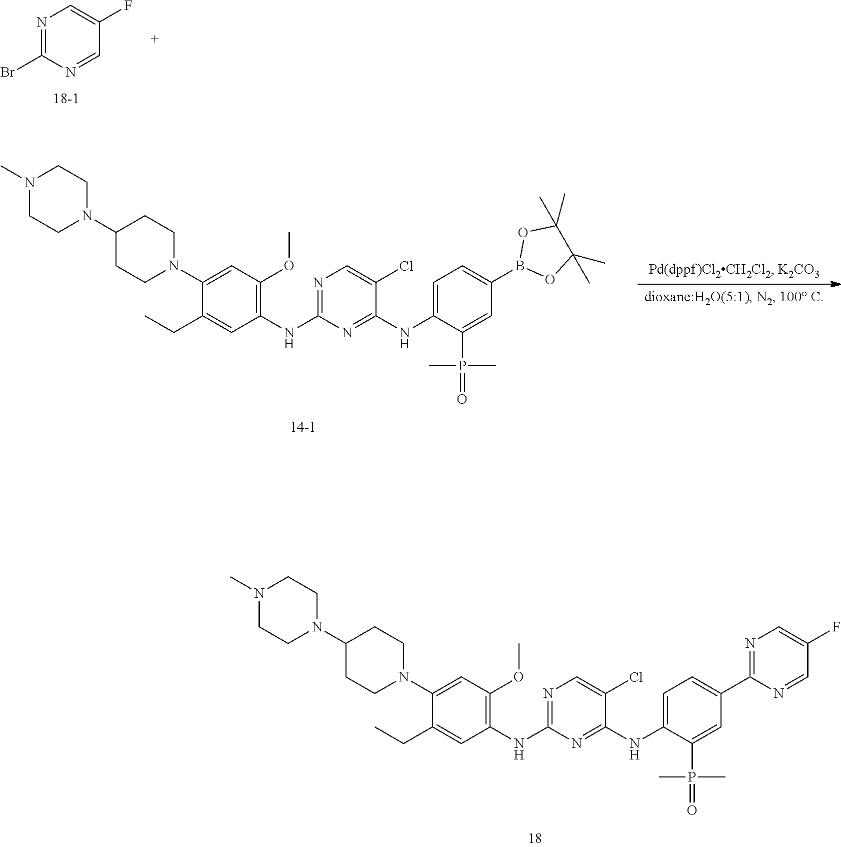

18-1 (15 mg), 14-1 (50 mg), K₂CO₃ (28 mg), Pd(dppf)Cl₂.CH₂Cl₂ (5 mg), 1,4-dioxane (5 mL) and water (1 mL) was added successively to the reaction flask, and mixed well followed by nitrogen protection. Raise temperature to 100° C., stir with heating overnight. The reaction was complete as monitored by LCMS, and then stopped. The reaction mixture was filtered with suction to remove solid granules, concentrated to remove solvent, and purified by preparative TCL (EtOAc:MeOH=1:1.5) to obtain the desired product 18 (19 mg), MS: 708.30 [M+H]⁺

### Example 125: Synthesis of Compound 19

### (2-((5-Chloro-2-((5-ethyl-2-methoxy-4-(4-(4-methylpiperazin-1-yl)piperidin-1-yl)phenyl)amino)pyrimidin-4-yl)amino)-5-(4-methoxypyrimidin-2-yl)phenyl)dimethylphosphine oxide

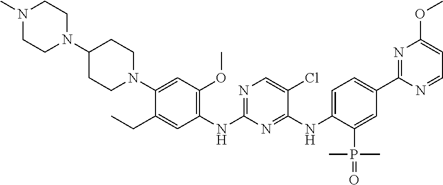

### Step 1: Synthesis of Compound 19

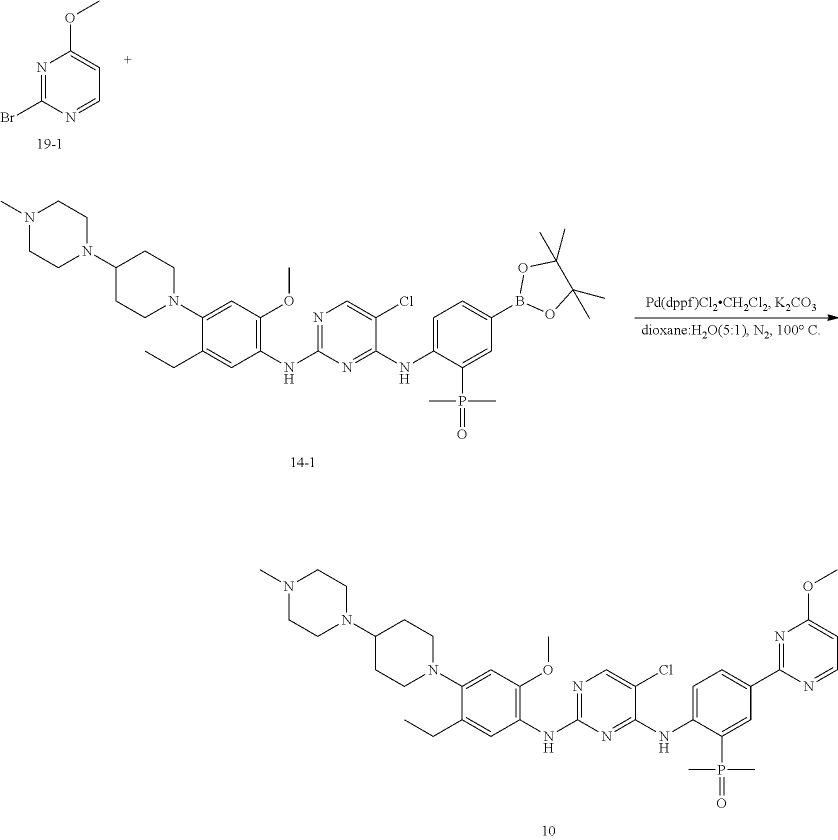

The compound 19 was synthesized as the method described for compound 18 using compound 19-1 instead of raw compound 18-1. For compound 19, MS: 702.32 [M+H]⁺

### Example 126: Synthesis of Compound 20

### (2-((5-Chloro-2-((5-ethyl-2-methoxy-4-(4-(4-methylpiperazin-1-yl)piperidin-1-yl)phenyl)amino)pyrimidin-4-yl)amino)-5-(5-methoxypyrimidin-2-yl)phenyl)dimethylphosphine oxide

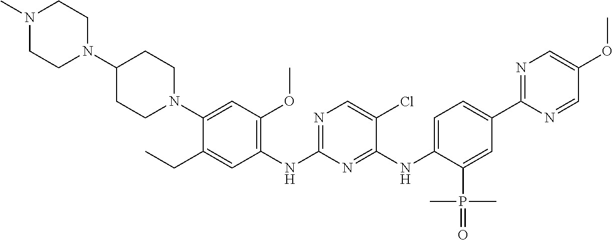

### Step 1: Synthesis of Compound 20

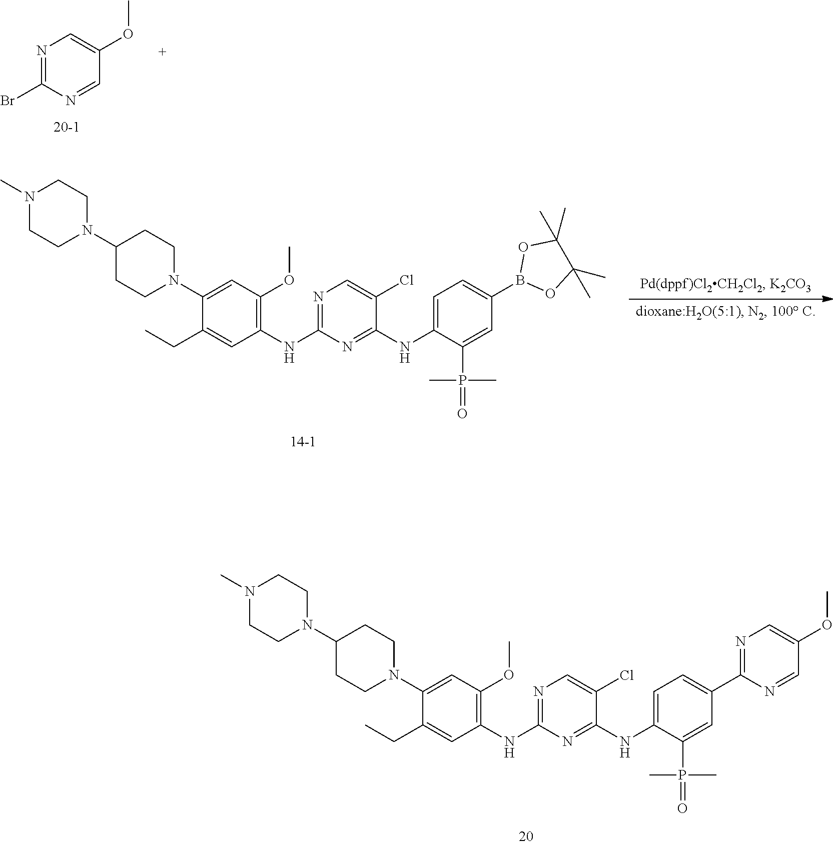

The compound 20 was synthesized as the method described for compound 18 using compound 20-1 instead of raw compound 18-1. For compound 20, MS: 720.32 [M+H]⁺

### Example 127: Synthesis of Compound 21

### (2-((5-Bromo-2-((5-ethyl-2-methoxy-4-(4-(4-methylpiperazin-1-yl)piperidin-1-yl)phenyl)amino)pyrimidin-4-yl)amino)-5-(5-methylpyrimidin-2-yl)phenyl)dimethylphosphine oxide

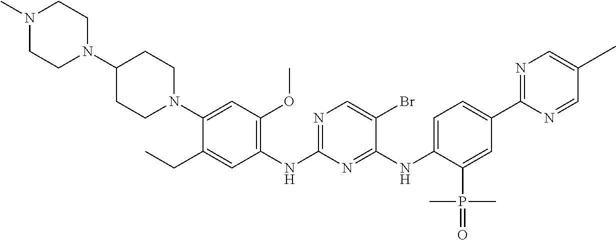

### Step 1: Synthesis of Compound 21-2

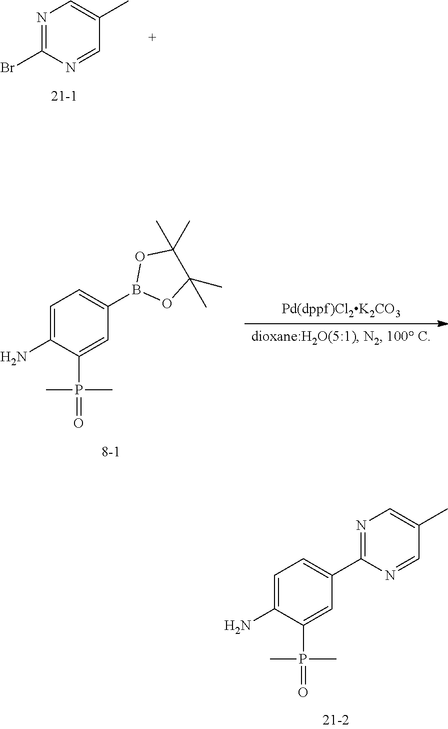

The compound 21-2 was synthesized as the method described for compound 8-3 using compound 21-1 instead of raw compound 8-2. For compound 21-2, MS: 262.10 [M+H]⁺

### Step 2: Synthesis of Compound 21-3

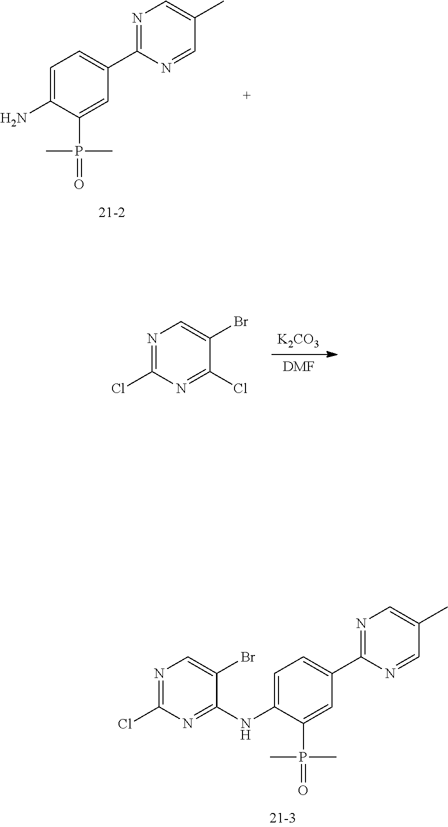

The compound 21-3 was synthesized as the method described for compound 1-10 using compound 21-2 instead of raw compound 1-8. For compound 21-3, MS: 452.00 [M+H]⁺

### Step 3: Synthesis of Compound 21

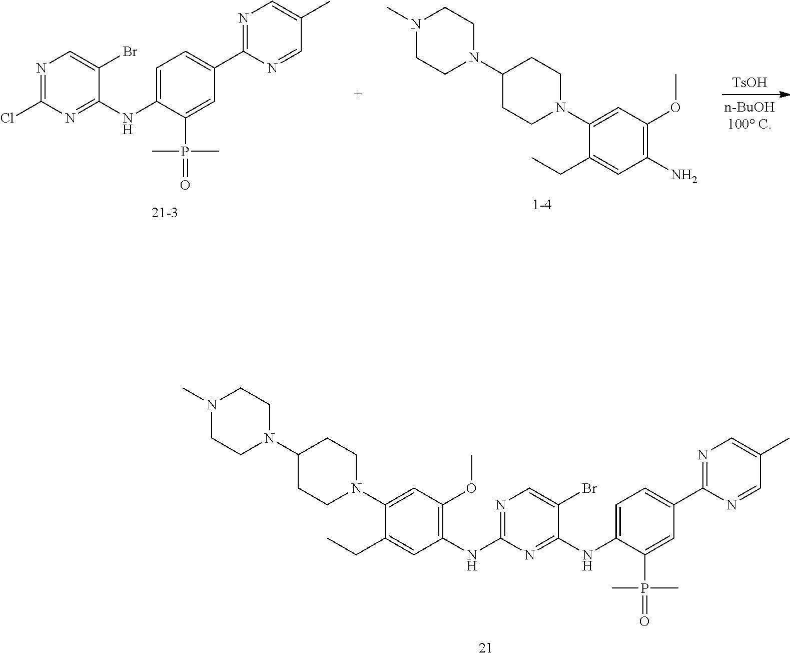

The compound 21 was synthesized as the method described for compound 1 using compound 21-3 instead of raw compound 1-10. For compound 21, MS: 748.28 [M+H]⁺

### Example 128: Synthesis of Compound 22

### (2-((5-Bromo-2-((5-ethyl-2-methoxy-4-(4-(4-methylpiperazin-1-yl)piperidin-1-yl)phenyl)amino)pyrimidin-4-yl)amino)-5-(pyrimidin-5-yl)phenyl)dimethylphosphine oxide

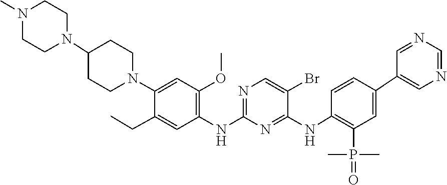 [SMILES: CCc1cc(Nc2ncc(Br)c(Nc3ccc(-c4cncnc4)cc3P(C)(C)=O)n2)c(OC)cc1N1CCC(N2CCN(C)CC2)CC1]

### Step 1: Synthesis of Compound 22-2

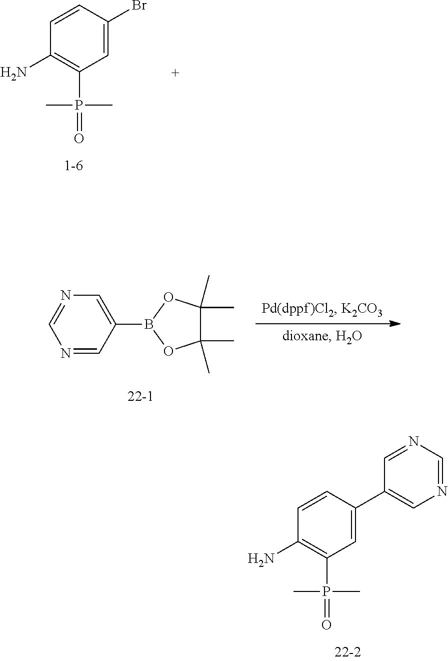 [SMILES: CP(C)(=O)c1cc(Br)ccc1N ; CC1(C)OB(c2cncnc2)OC1(C)C ; "Pd(dppf)Cl2, K2CO3" ; "dioxane, H2O" ; CP(C)(=O)c1cc(-c2cncnc2)ccc1N]

Add compounds 1-6 (0.6 g), 22-1 (0.75 g), K₂CO₃ (0.67 g), Pd(dppf)Cl₂ (0.2 g), dioxane (20 ml) and water (4 ml) successively to the reaction flask, heat to 100° C. under nitrogen protection, and stir with heating for 12 hr. The reaction was complete as monitored by LCMS, and then stopped. After adding water (50 mL), the reaction solution was extracted with dichloromethane (3×50 mL); the organic phase was washed with saturated brine (3×30 mL), dried over anhydrous sodium sulfate, separated and purified by column chromatography (dichloromethane:methanol=20:1). The solvent was removed to obtain the brown solid of desired product 22-2 (1.0 g), MS: 248.09 [M+H]⁺

### Step 2: Synthesis of Compound 22-3

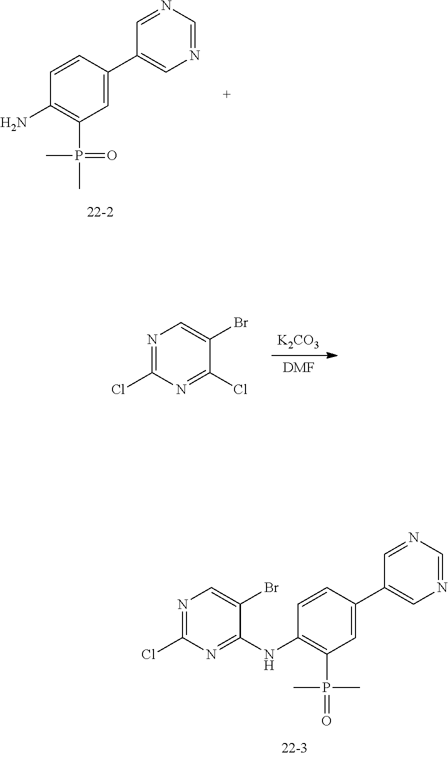

Add compound 22-2 (1.0 g), 5-Bromo-2,4-dichloropyrimidine (1.9 g), K₂CO₃ (2.0 g) and DMF (20 ml) successively into the reaction flask, heat to 50° C., and stir with heating for 12 hr. The reaction was complete as monitored by LCMS, and then stopped. The reaction solution was poured into water (50 ml), and extracted with ethyl acetate (3×50 mL); the organic phase was washed with saturated brine (3×30 mL), dried over anhydrous sodium sulfate, separated and purified by column chromatography (dichloromethane:methanol=20:1). The solvent was removed to obtain the yellowish solid of desired compound 22-3 (700 mg), MS: 437.98 [M+H]⁺

### Step 3: Synthesis of Compound 22

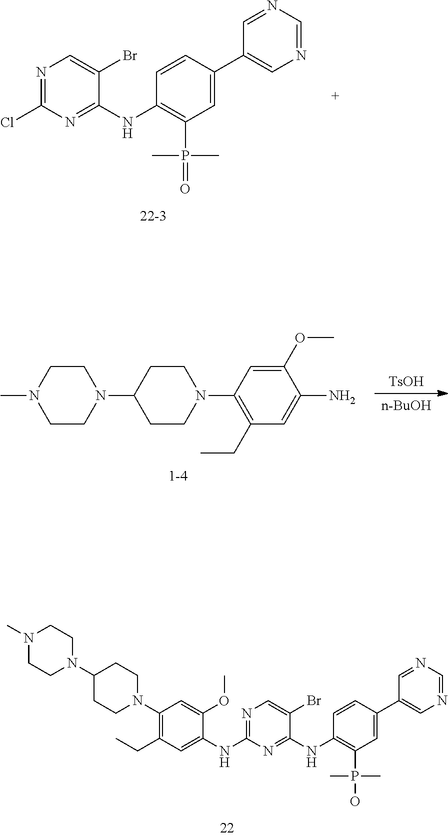

Add compound 22-3 (100 mg), 1-4 (113 mg), p-toluenesulfonic acid (118 mg) and n-butanol (10 ml) successively into the reaction flask, heat to 120° C. and stir for 12 hr. The reaction was complete as monitored by LCMS, and then stopped. The reaction solution was poured into sodium carbonate solution (5 mL), and extracted with dichloromethane (3×50 mL); the organic phase was washed with saturated brine (3×30 mL), dried over anhydrous sodium sulfate, separated and purified by preparative TLC (dichloromethane:methanol=8:1), and then concentrated to obtain yellowish solid of compound 22 (40 mg), MS: 734.26 [M+H]⁺

### Example 129: Synthesis of Compound 23

### (2-((5-Chloro-2-((5-ethyl-2-methoxy-4-(4-(4-methylpiperazin-1-yl)piperidin-1-yl)phenyl)amino)pyrimidin-4-yl)amino)-5-(pyrazolo[1,5-c]pyrimidin-3-yl)phenyl)dimethylphosphine oxide

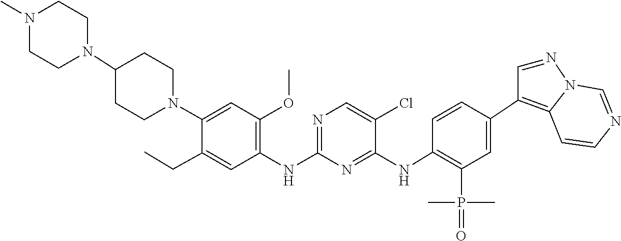 [SMILES: CCc1cc(Nc2ncc(Cl)c(Nc3ccc(-c4cnn5cnccc45)cc3P(C)(C)=O)n2)c(OC)cc1N1CCC(N2CCN(C)CC2)CC1]

### Step 1: Synthesis of Compound 23

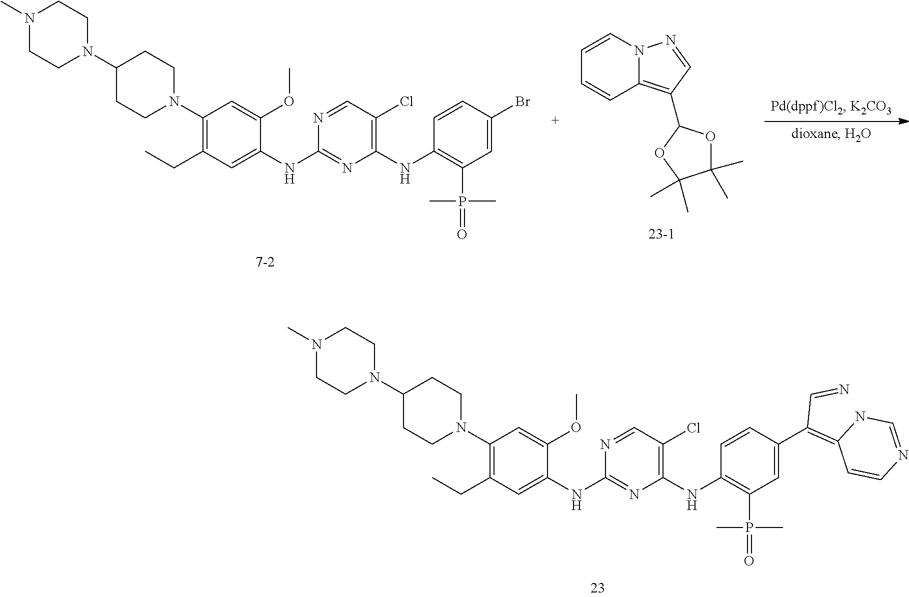 [SMILES: CCc1cc(Nc2ncc(Cl)c(Nc3ccc(Br)cc3P(C)(C)=O)n2)c(OC)cc1N1CCC(N2CCN(C)CC2)CC1 ; CC1(C)OC(c2cnn3ccccc23)OC1(C)C ; "Pd(dppf)Cl2, K2CO3" ; "dioxane, H2O" ; CCc1cc(Nc2ncc(Cl)c(Nc3ccc(/C(C=N)=C4\C=CN=CN4)cc3P(C)(C)=O)n2)c(OC)cc1N1CCC(N2CCN(C)CC2)CC1]

Add compounds 7-2 (50 mg), 23-1 (20 mg), Pd(dppf)Cl₂ (6 mg), K₂CO₃ (20 mg), dioxane (4 ml) and water (1 ml) successively to the reaction flask, raise temperature to 100° C. under nitrogen protection and stir with heating for 12 hr. The reaction was complete as monitored by LCMS, and then stopped. After removing the solvent, the residue was separated and purified by the preparative TLC (dichloromethane:methanol=10:1) to obtain the yellowish solid of desired compound 23 (24.7 mg), MS: 729.32 [M+H]⁺

### Example 130: Synthesis of Compound 24

### (2-((5-Chloro-2-((5-ethyl-2-methoxy-4-(4-(4-methylpiperazin-1-yl)piperidin-1-yl)phenyl)amino)pyrimidin-4-yl)amino)-5-(2-methylpyrimidin-5-yl)phenyl)dimethylphosphine oxide

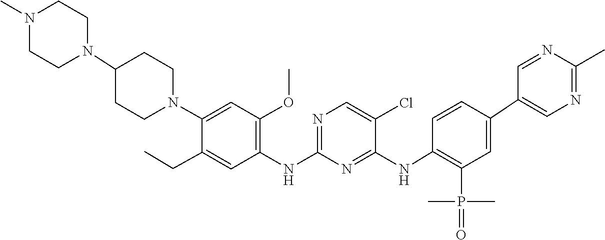

### Step 1: Synthesis of Compound 24

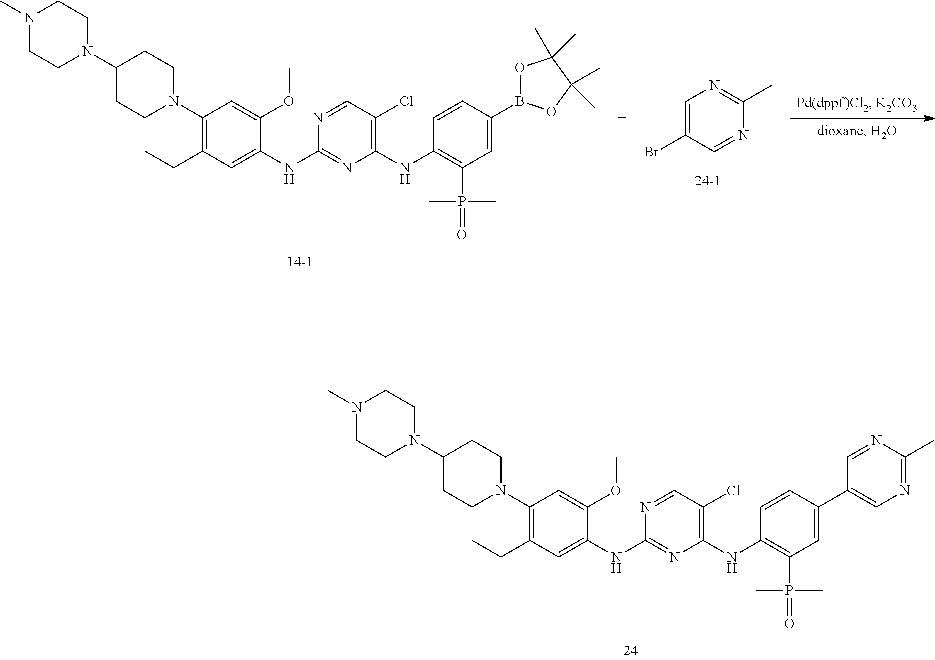

Add compounds 14-1 (50 mg), 24-1 (24 mg), Pd(dppf)Cl₂ (6 mg), K₂CO₃ (20 mg), dioxane (4 mL) and water (1 ml) successively to the reaction flask, raise temperature to 100° C. under nitrogen protection and stir with heating for 12 hr. The reaction was complete as monitored by LCMS, and then stopped. After removing the solvent, the residue was separated and purified by the preparative TLC (dichloromethane:methanol=10:1) to obtain the yellowish solid of desired compound 24 (9.1 mg), MS: 704.33 [M+H]⁺

### Example 131: Synthesis of Compound 25

### (2-((5-Chloro-2-((5-ethyl-2-methoxy-4-(4-(4-methylpiperazin-1-yl)piperidin-1-yl)phenyl)amino)pyrimidin-4-yl)amino)-5-(2-methoxypyrimidin-5-yl)phenyl)dimethylphosphine oxide

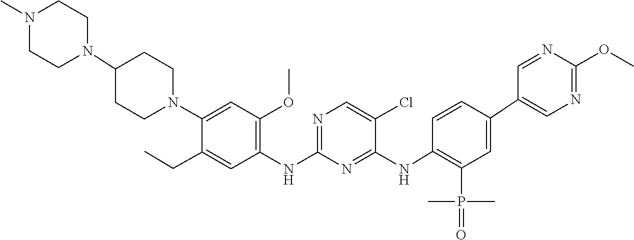

### Step 1: Synthesis of Compound 25

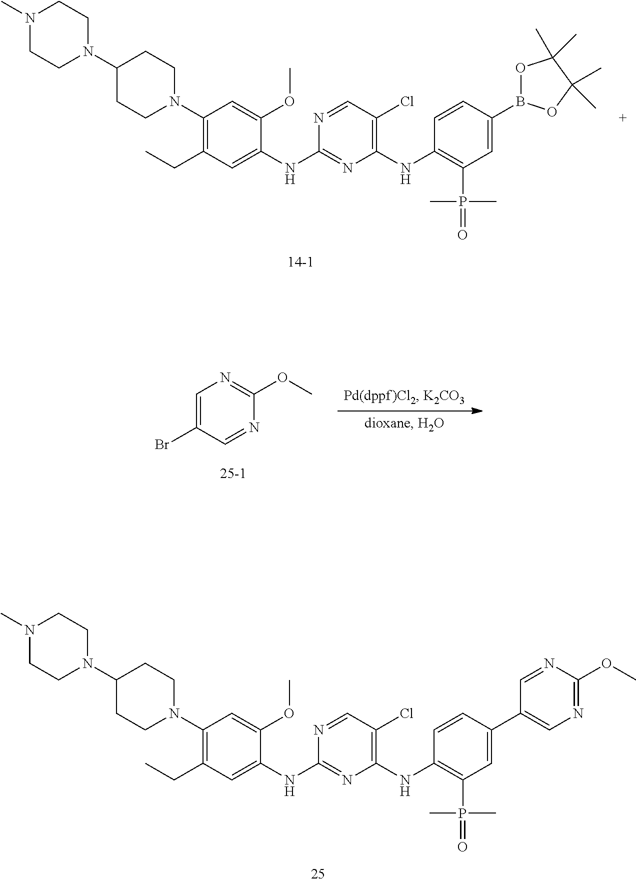

Add compounds 14-1 (50 mg), 25-1 (27 mg), Pd(dppf)Cl₂ (6 mg), K₂CO₃ (20 mg), dioxane (4 mL) and water (1 mL) successively to the reaction flask, raise temperature to 100° C. under nitrogen protection and stir with heating for 12 hr. The reaction was complete as monitored by LCMS, and then stopped. After removing the solvent, the residue was separated and purified by the preparative TLC (dichloromethane:methanol=10:1) to obtain the yellowish solid of desired compound 25 (21.5 mg), MS: 720.32 [M+H]+

### Example 132: Synthesis of Compound 26

### (2-((5-Bromo-2-((5-ethyl-2-methoxy-4-(4-(4-methylpiperazin-1-yl)piperidin-1-yl)phenyl)amino)pyrimidin-4-yl)amino)-5-(pyrimidin-2-yl)phenyl)dimethylphosphine oxide

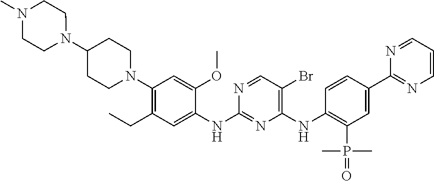 [SMILES: CCc1cc(Nc2ncc(Br)c(Nc3ccc(-c4ncccn4)cc3P(C)(C)=O)n2)c(OC)cc1N1CCC(N2CCN(C)CC2)CC1]

### Step 1: Synthesis of Compound 26-2

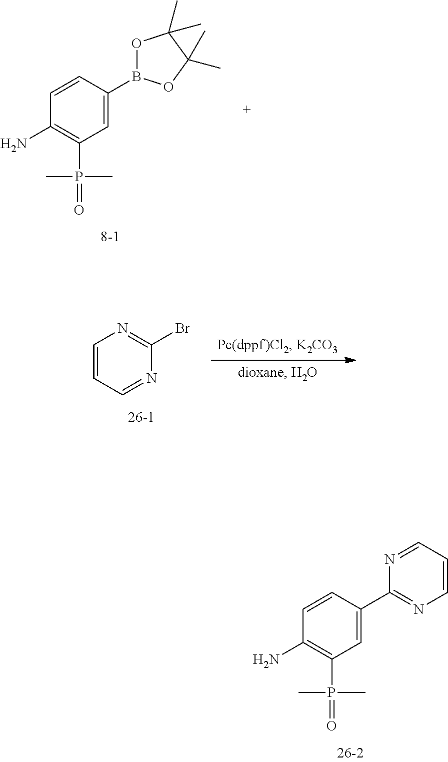 [SMILES: CC1(C)OB(c2ccc(N)c(P(C)(C)=O)c2)OC1(C)C ; Brc1ncccn1 ; "Pc(dppf)Cl2, K2CO3" ; "dioxane, H2O" ; CP(C)(=O)c1cc(-c2ncccn2)ccc1N]

The compound 26-2 was synthesized as the method described for compound 8-3 using compound 26-1 instead of raw compound 8-2. For compound 26-2, MS: 248.09 [M+H]⁺

### Step 2: Synthesis of Compound 26-3

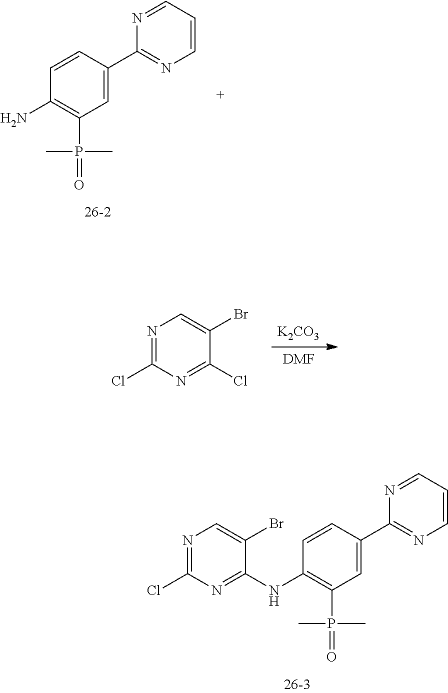

The compound 26-3 was synthesized as the method described for compound 1-10 using compound 26-2 instead of raw compound 1-8. For compound 26-3, MS: 437.98 [M+H]⁺

### Step 3: Synthesis of Compound 26

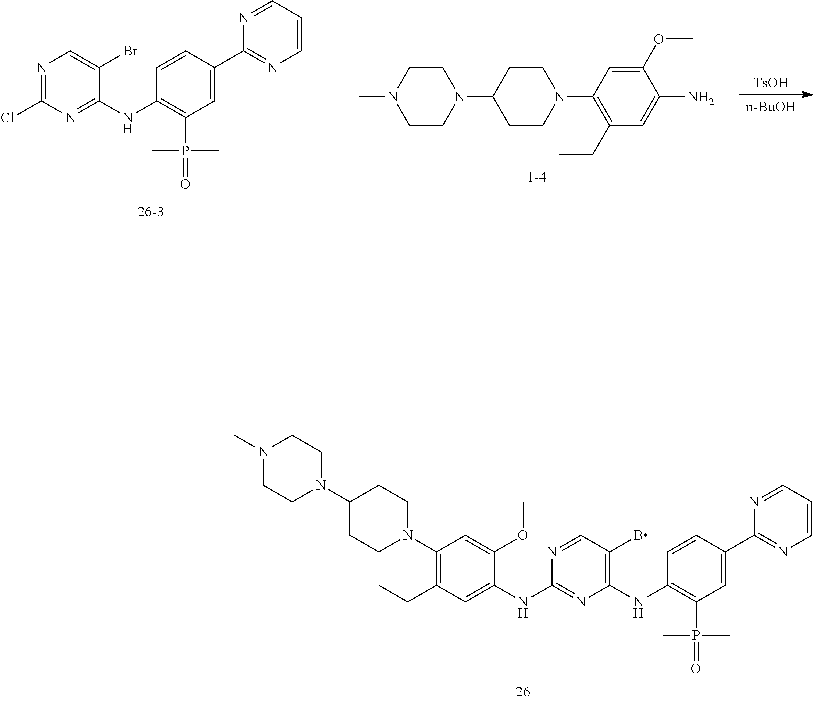

The compound 26 was synthesized as the method described for compound 1 using compound 26-3 instead of raw compound 1-10. For compound 26, MS: 734.26 [M+H]⁺

### Example 133: Synthesis of Compound 27

### (2-((5-Bromo-2-((5-ethyl-2-methoxy-4-(4-(4-methylpiperidin-1-yl)piperidin-1-yl)phenyl))amino)pyrimidin-4-yl)amino)-5-(oxazol-2-yl)phenyl)dimethylphosphine oxide

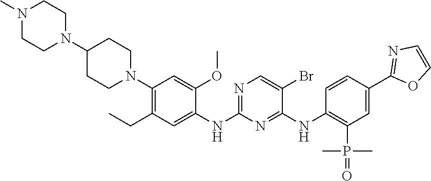 [SMILES: CCc1cc(Nc2ncc(Br)c(Nc3ccc(-c4ncco4)cc3P(C)(C)=O)n2)c(OC)cc1N1CCC(N2CCN(C)CC2)CC1]

### Step 1: Synthesis of Compound 27-2

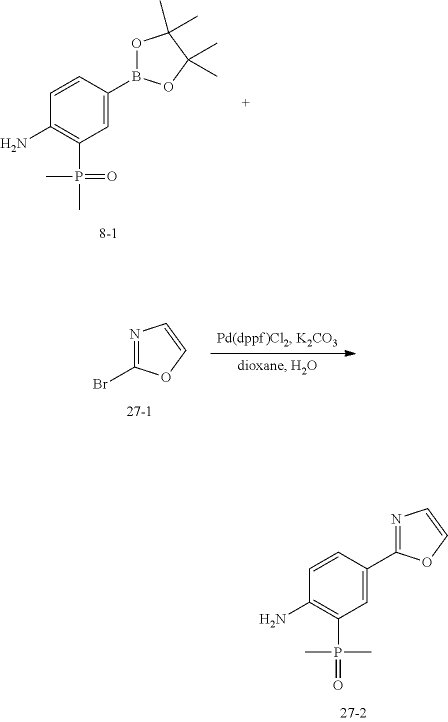 [SMILES: CC1(C)OB(c2ccc(N)c(P(C)(C)=O)c2)OC1(C)C ; Brc1ncco1 ; "Pd(dppf)Cl2, K2CO3" ; "dioxane, H2O" ; CP(C)(=O)c1cc(-c2ncco2)ccc1N]

The compound 27-2 was synthesized as the method described for compound 8-3 using compound 27-1 instead of raw compound 8-2. For compound 27-2, MS: 237.07 [M+H]⁺

### Step 2: Synthesis of Compound 27-3

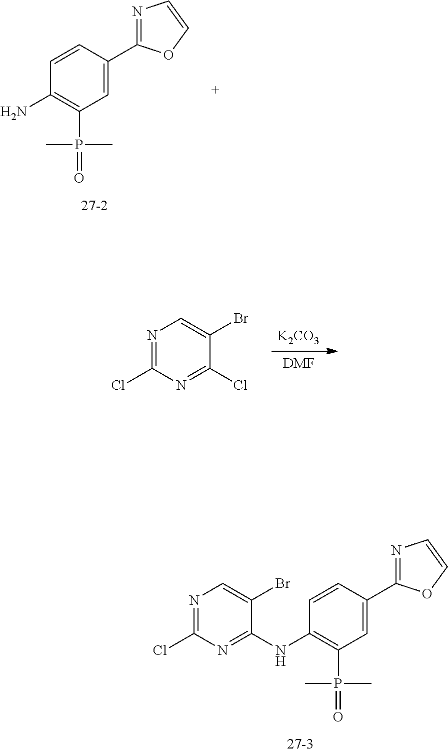

The compound 27-3 was synthesized as the method described for compound 1-10 using compound 27-2 instead of raw compound 1-8. For compound 27-3, MS: 426.% [M+H]⁺

### Step 3: Synthesis of Compound 27

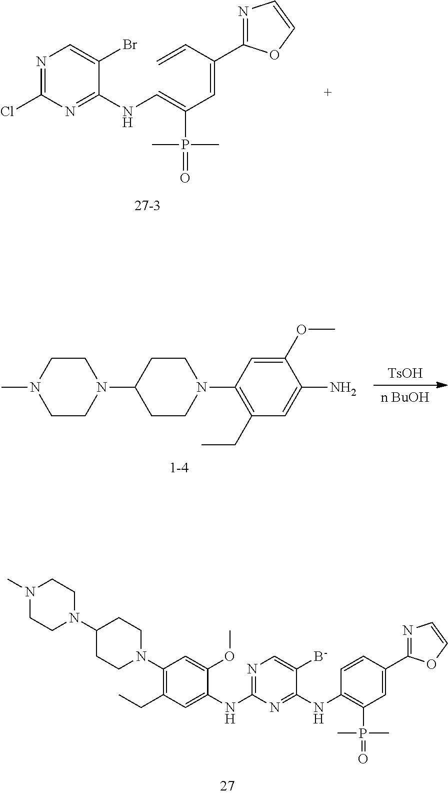

The compound 27 was synthesized as the method described for compound 1 using compound 27-3 instead of raw compound 1-10. For compound 27, MS: 723.25 [M+H]⁺

### Example 134: Synthesis of Compound 28

### (2-((5-Bromo-2-((5-ethyl-2-methoxy-4-(4-(4-methylpiperazin-1-yl)piperidin-1-yl)phenyl)amino)pyrimidin-4-yl)amino)-5-(thiazol-4-yl)phenyl)dimethylphosphine oxide

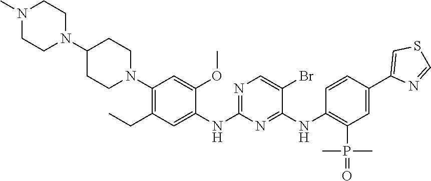 [SMILES: CCc1cc(Nc2ncc(Br)c(Nc3ccc(-c4cscn4)cc3P(C)(C)=O)n2)c(OC)cc1N1CCC(N2CCN(C)CC2)CC1]

### Step 1: Synthesis of Compound 28-2

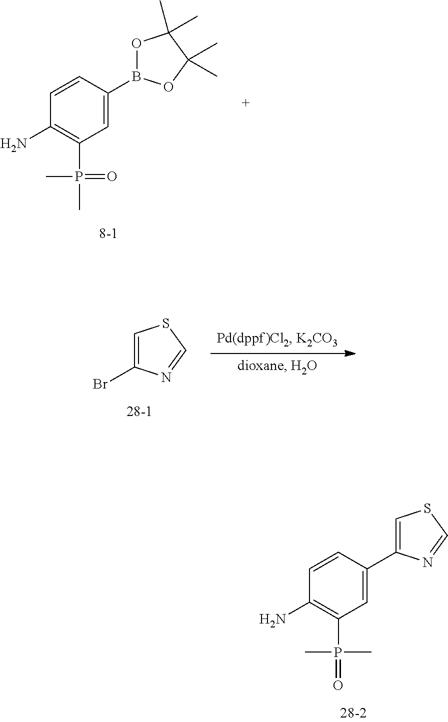 [SMILES: CC1(C)OB(c2ccc(N)c(P(C)(C)=O)c2)OC1(C)C ; Brc1cscn1 ; "Pd(dppf)Cl2, K2CO3" ; "dioxane, H2O" ; CP(C)(=O)c1cc(-c2cscn2)ccc1N]

The compound 28-2 was synthesized as the method described for compound 8-3 using compound 28-1 instead of raw compound 8-2. For compound 28-2, MS: 253.05 [M+H]⁺

### Step 2: Synthesis of Compound 28-3

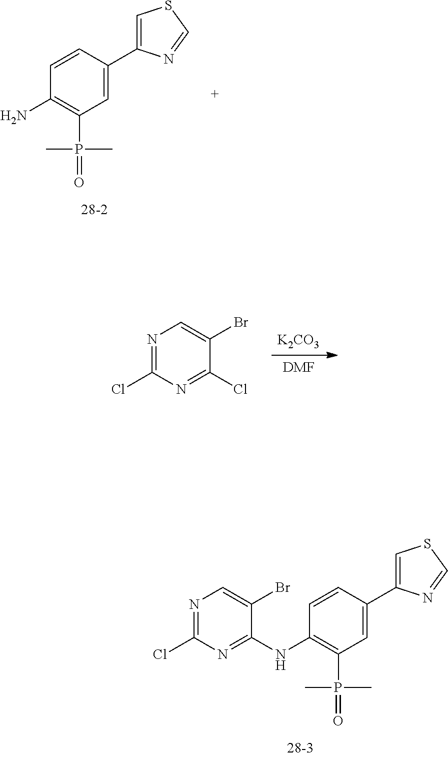

The compound 28-3 was synthesized as the method described for compound 1-10 using compound 28-2 instead of raw compound 1-8. For compound 28-3, MS: 442.94 [M+H]⁺

### Step 3: Synthesis of Compound 28

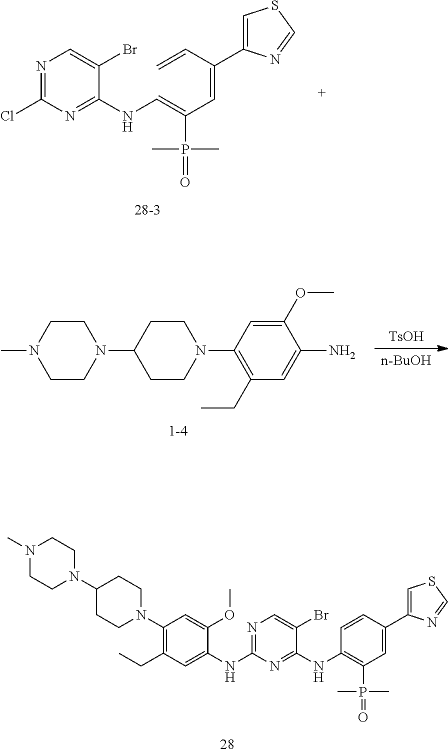

The compound 28 was synthesized as the method described for compound 1 21 using compound 28-3 instead of raw compound 1-10.

For compound 28, MS: 739.22 [M+H]⁺

¹H NMR (500 MHz, DMSO-d6) δ 11.04 (s, 1H), 9.39 (s, 1H), 9.09 (s, 1H), 8.35 (s, 1H), 8.18 (d, J=13.0 Hz, 2H), 7.90 (d, J=14.3 Hz, 1H), 7.71 (d, J=8.4 Hz, 1H), 7.42 (s, 1H), 6.81 (s, 1H), 3.78 (s, 3H), 2.94 (d, J=51.1 Hz, 4H), 2.75-2.54 (m, 4H), 2.53 (s, 3H), 2.50 (m, 6H), 2.37 (s, 1H), 1.85 (d, J=13.6 Hz, 6H), 1.76 (d, J=16.3 Hz, 2H), 1.56 (s, 2H), 1.00 (t, J=7.5 Hz, 3H).

### Example 135: Synthesis of Compound 29

### (2-((5-Bromo-2-((5-ethyl-2-methoxy-4-(4-(4-methylpiperazin-1-yl)piperidin-1-yl)phenyl)amino)pyrimidin-4-yl)amino)-5-(5-fluoropyrimidin-2-yl)phenyl)dimethylphosphine oxide

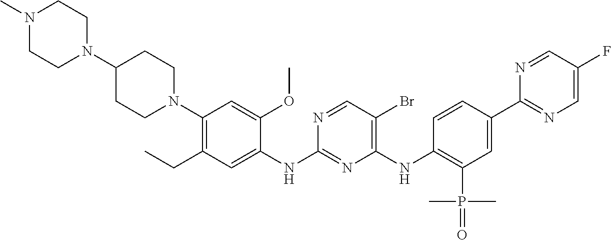 [SMILES: CCc1cc(Nc2ncc(Br)c(Nc3ccc(-c4ncc(F)cn4)cc3P(C)(C)=O)n2)c(OC)cc1N1CCC(N2CCN(C)CC2)CC1]

### Step 1: Synthesis of Compound 29-2

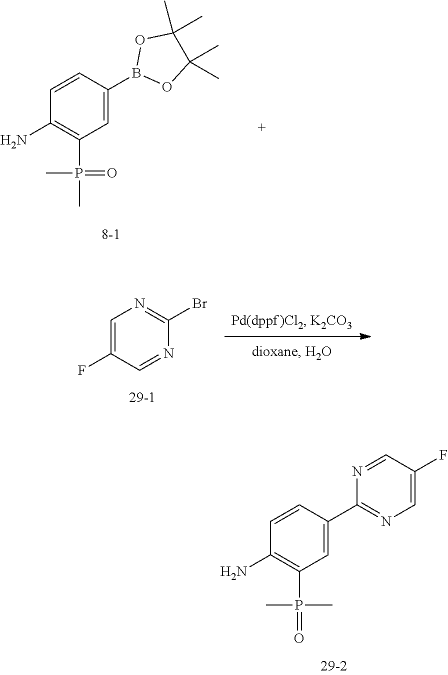 [SMILES: CC1(C)OB(c2ccc(N)c(P(C)(C)=O)c2)OC1(C)C ; Fc1cnc(Br)nc1 ; CP(C)(=O)c1cc(-c2ncc(F)cn2)ccc1N]

The compound 29-2 was synthesized as the method described for compound 8-3 using compound 29-1 instead of raw compound 8-2. For compound 29-2, MS: 266.08 [M+H]⁺

### Step 2: Synthesis of Compound 29-3

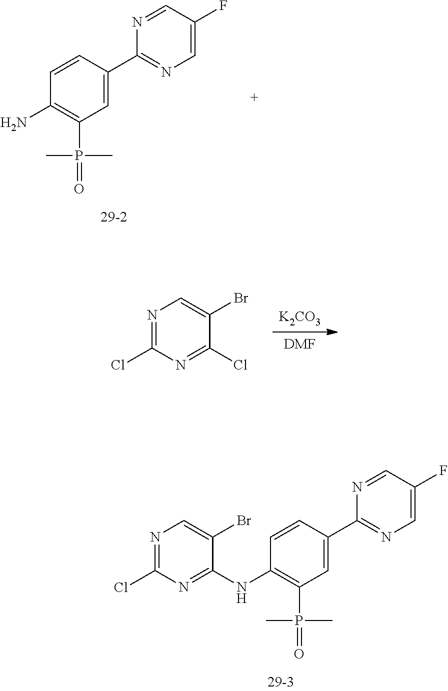

The compound 29-3 was synthesized as the method described for compound 1-10 using compound 29-2 instead of raw compound 1-8. For compound 29-3, MS: 455.97 [M+H]⁺

### Step 3: Synthesis of Compound 29

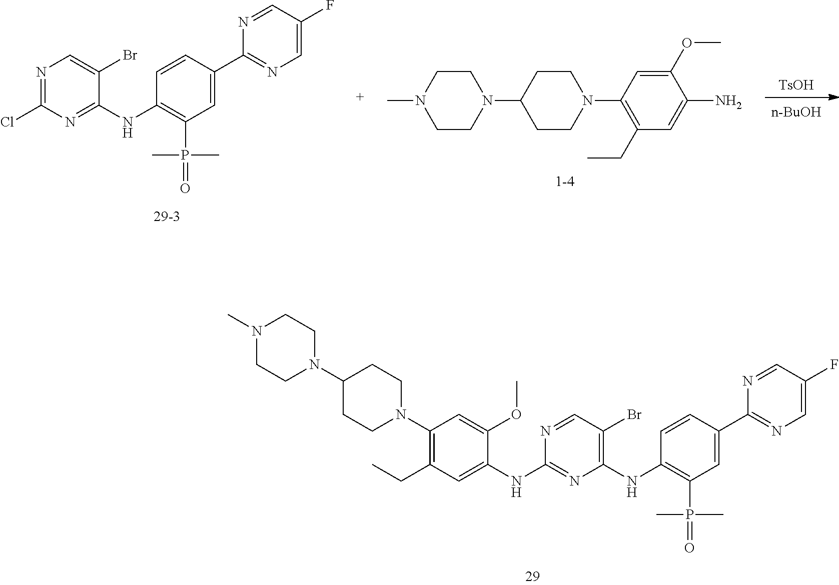

The compound 29 was synthesized as the method described for compound 1 using compound 29-3 instead of raw compound 1-10. For compound 29, MS: 752.25 [M+H]⁺

### Example 136: Synthesis of Compound 30

### (2-((5-Bromo-2 (-((5-ethyl-2-methoxy-4-(4-(4-methylpiperazin-1-yl)piperidin-1-yl)phenyl)amino)pyrimidin-4-yl)amino)-5-(5-methoxypyrimidin-2-yl)phenyl)dimethylphosphine oxide

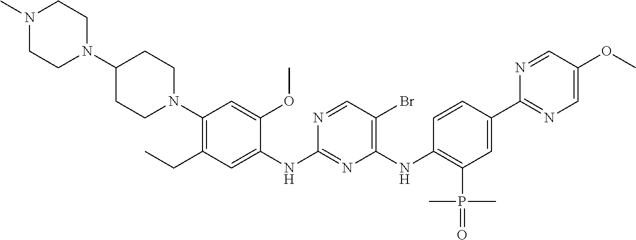

### Step 1: Synthesis of Compound 30-2

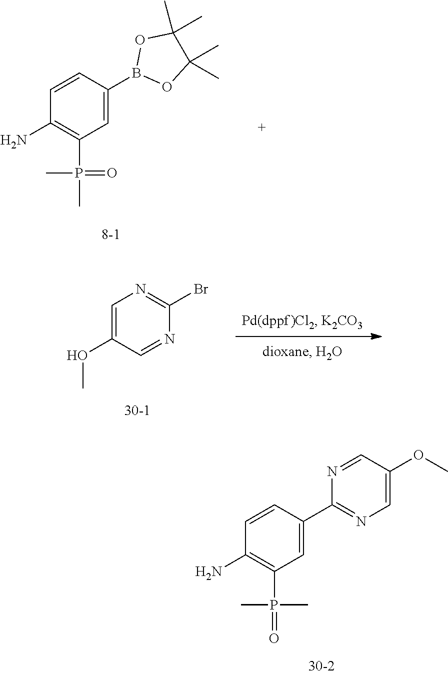

The compound 30-2 was synthesized as the method described for compound 8-3 using compound 30-1 instead of raw compound 8-2. For compound 30-2, MS: 278.10 [M+H]⁺

### Step 2: Synthesis of Compound 30-3

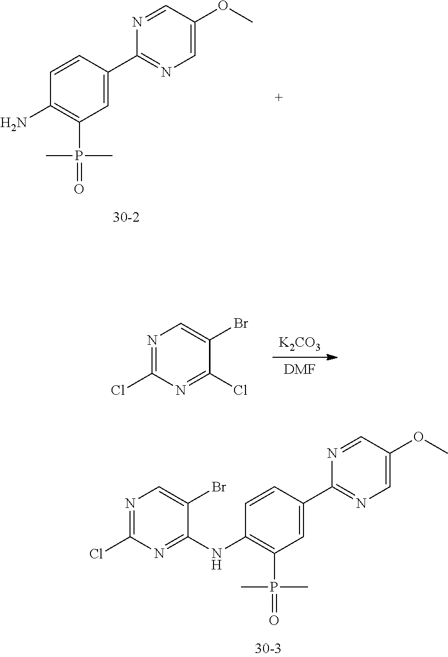

The compound 30-3 was synthesized as the method described for compound 1-10 using compound 30-2 instead of raw compound 1-8. For compound 30-3, MS: 467.99 [M+H]⁺

### Step 3: Synthesis of Compound 30

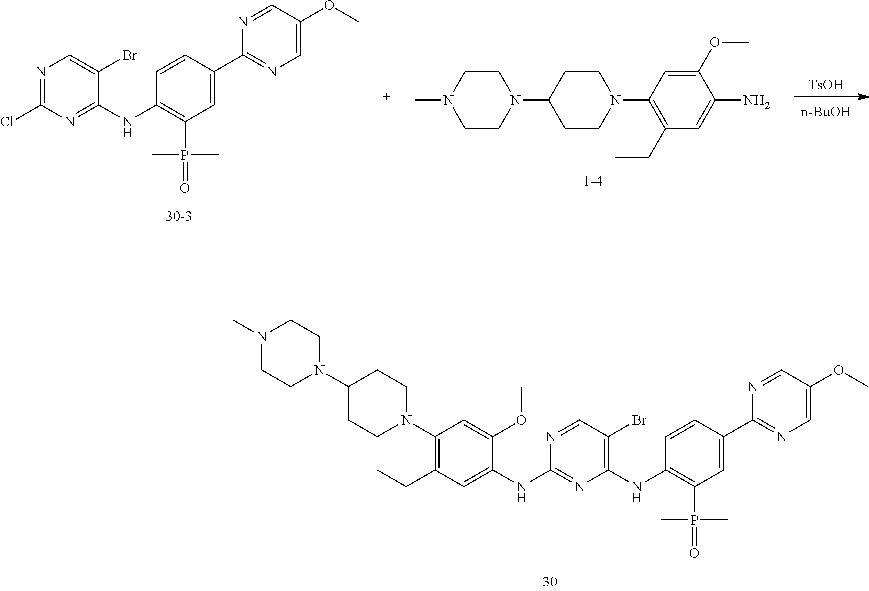

The compound 30 was synthesized as the method described for compound 1 using compound 30-3 instead of raw compound 1-10. For compound 30, MS: 764.27 [M+H]⁺

### Example 137: Synthesis of Compound 31

### (2-((5-Bromo-2-((5-ethyl-2-methoxy-4-(4-(4-methylpiperazin-1-yl)piperidin-1-yl)phenyl)amino)pyrimidin-4-yl)amino)-5-(1-methyl-1H-1,2,4-triazol-3-yl)phenyl)dimethylphosphine oxide

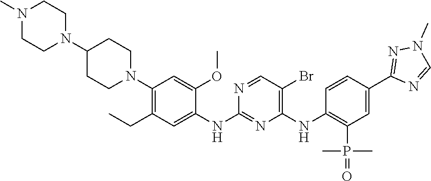 [SMILES: CCc1cc(Nc2ncc(Br)c(Nc3ccc(-c4ncn(C)n4)cc3P(C)(C)=O)n2)c(OC)cc1N1CCC(N2CCN(C)CC2)CC1]

### Step 1: Synthesis of Compound 31-2

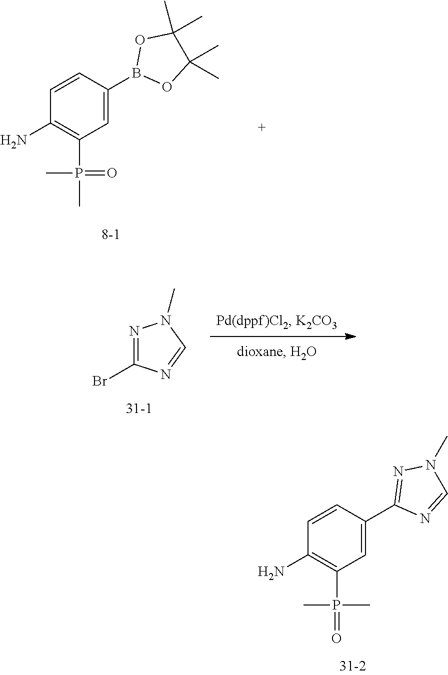

The compound 31-2 was synthesized as the method described for compound 8-3 using compound 31-1 instead of raw compound 8-2. For compound 31-2, MS: 251.10 [M+H]⁺

### Step 2: Synthesis of Compound 31-3

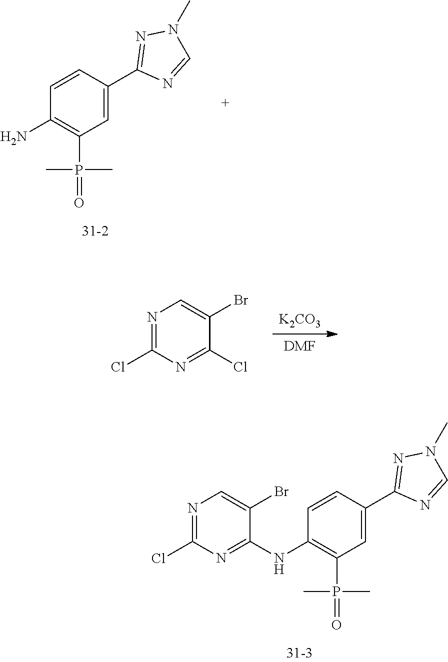

The compound 31-3 was synthesized as the method described for compound 1-10 using compound 31-2 instead of raw compound 1-8. For compound 31-3, MS: 440.99 [M+H]⁺

### Step 3: Synthesis of Compound 31

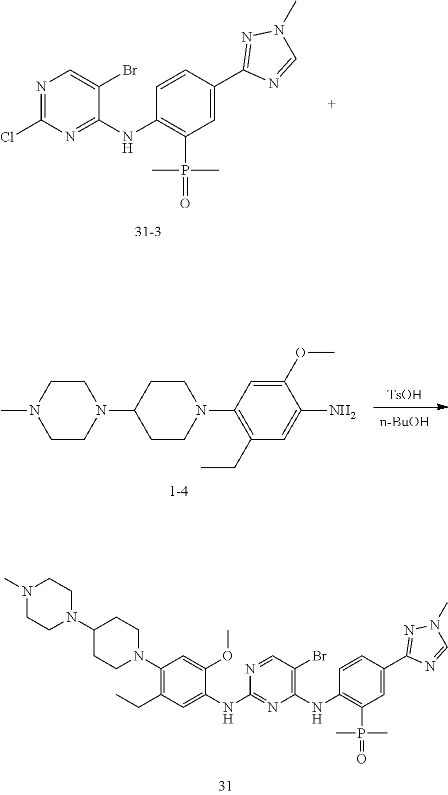

The compound 31 was synthesized as the method described for compound 1 using compound 31-3 instead of raw compound 1-10. For compound 31, MS: 737.27 [M+H]⁺

### Example 138: Synthesis of Compound 32

### (2-((5-Chloro-2-((5-ethyl-2-methoxy-4-(4-(4-methylpiperazin-1-yl)piperidin-1-yl)phenyl)amino)pyrimidin-4-yl)amino)-5-(pyrazin-2-yl)phenyl)dimethylphosphine oxide

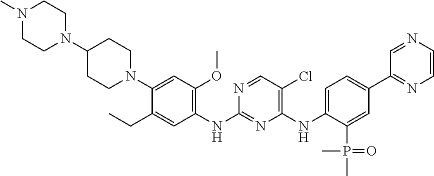 [SMILES: CCc1cc(Nc2ncc(Cl)c(Nc3ccc(-c4cnccn4)cc3P(C)(C)=O)n2)c(OC)cc1N1CCC(N2CCN(C)CC2)CC1]

### Step 1: Synthesis of Compound 32

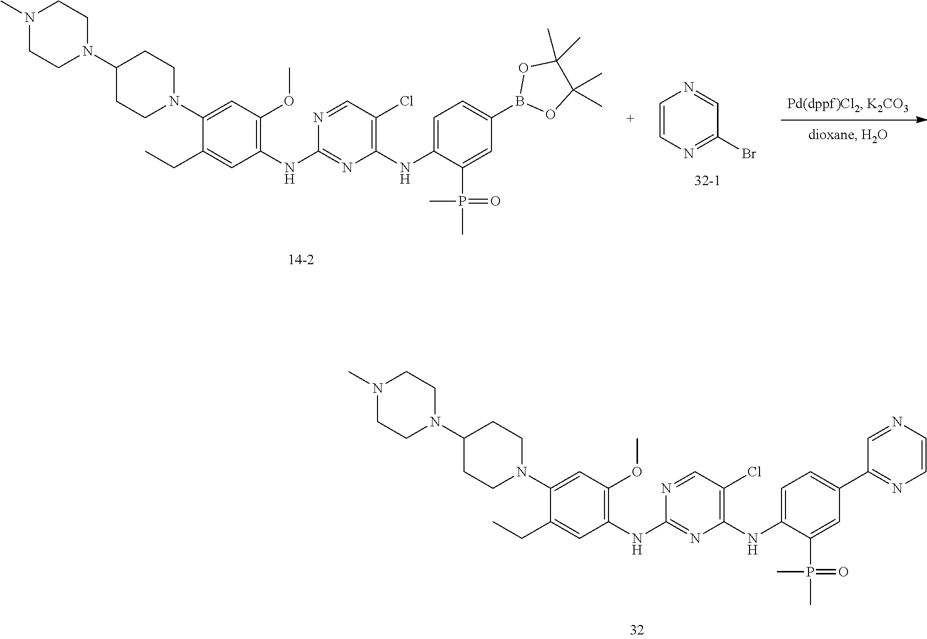

The compound 32 was synthesized as the method described for compound 14 using compound 32-1 instead of raw compound 14-2. For compound 32, MS: 690.31 [M+H]⁺

### Example 139: Synthesis of Compound 33

### (2-((5-Bromo-2-((5-ethyl-2-methoxy-4-(4-(4-methylpiperazin-1-yl)piperidin-1-yl)phenyl)amino)pyrimidin-4-yl)amino)-5-(6-methylpyridazin-3-yl)phenyl)dimethylphosphine oxide

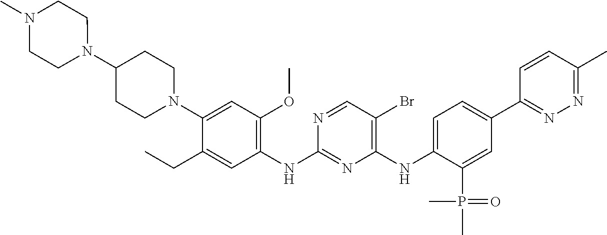

### Step 1: Synthesis of Compound 33-2

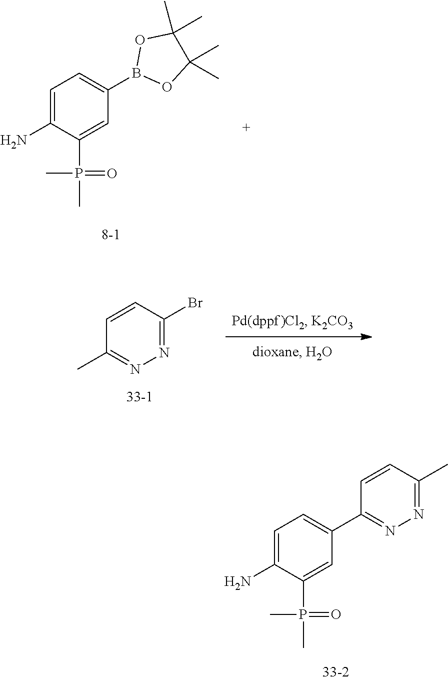

The compound 33-2 was synthesized as the method described for compound 8-3 using compound 33-1 instead of raw compound 8-2. For compound 33-2, MS: 262.10 [M+H]⁺

### Step 2: Synthesis of Compound 33-3

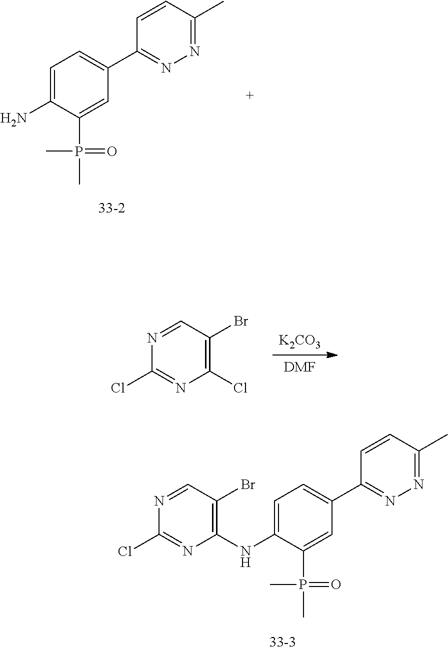

The compound 33-3 was synthesized as the method described for compound 1-10 using compound 33-2 instead of raw compound 1-8. For compound 33-3, MS: 452.00 [M+H]⁺

### Step 3: Synthesis of Compound 33

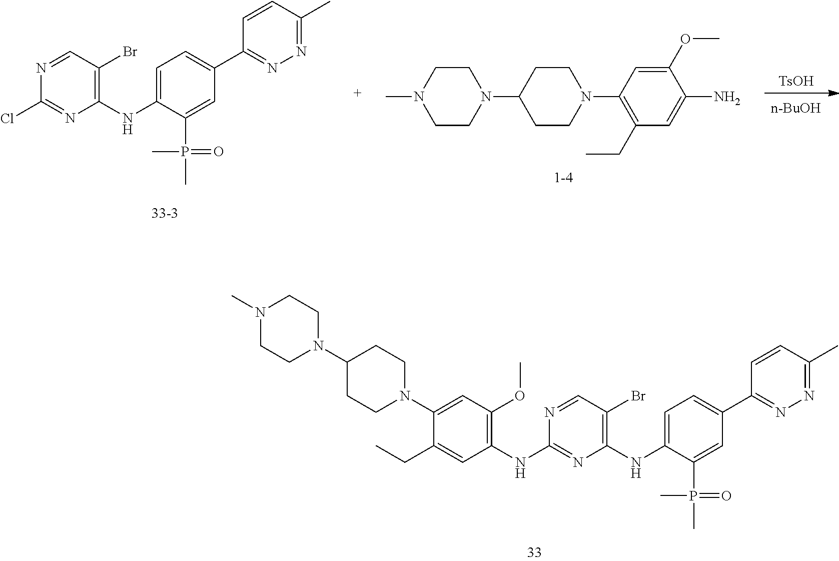

The compound 33 was synthesized as the method described for compound 1 using compound 33-3 instead of raw compound 1-10. For compound 33, MS: 748.28 [M+H]⁺

### Example 140: Synthesis of Compound 34

### (2-((5-Bromo-2-((5-ethyl-2-methoxy-4-(4-(4-methylpiperazin-1-yl)piperidin-1-yl)phenyl)amino)pyrimidin-4-yl)amino)-5-(2-methoxypyrimidin-5-yl)phenyl)dimethylphosphine oxide

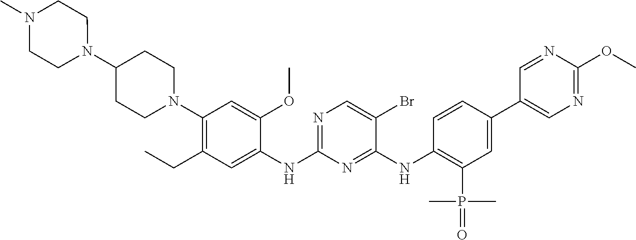

### Step 1: Synthesis of Compound 34-2

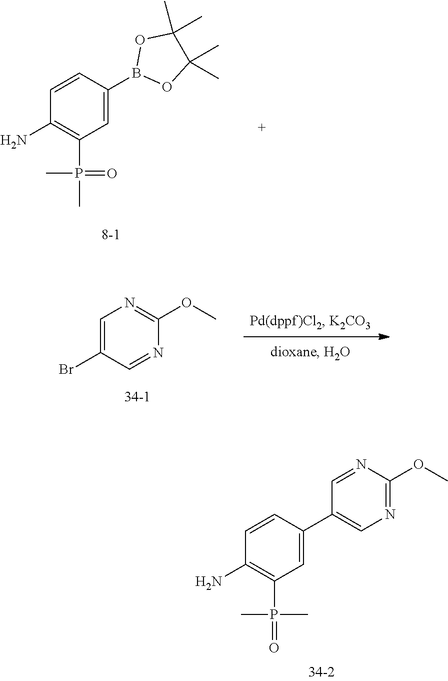

The compound 34-2 was synthesized as the method described for compound 8-3 using compound 34-1 instead of raw compound 8-2. For compound 34-2, MS: 278.10 [M+H]⁺

### Step 2: Synthesis of Compound 34-3

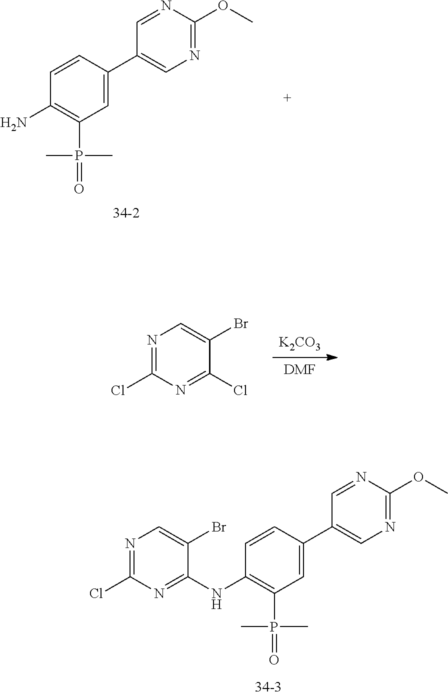

The compound 34-3 was synthesized as the method described for compound 1-10 using compound 34-2 instead of raw compound 1-8. For compound 34-3, MS: 467.99 [M+H]⁺

### Step 3: Synthesis of Compound 34

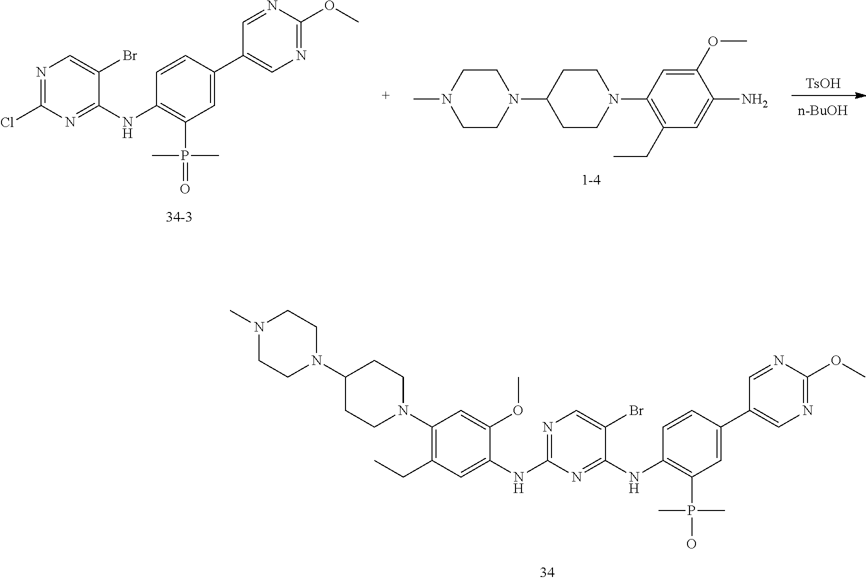

The compound 34 was synthesized as the method described for compound 1 using compound 34-3 instead of raw compound 1-10, the stirring time for reaction was changed to 3 hr. For compound 34, MS: 764.27 [M+H]⁺

### Example 141: Synthesis of Compound 35

### (2-((5-Bromo-2-((5-ethyl-2-methoxy-4-(4-(4-methylpiperazin-1-yl)piperidin-1-yl)phenyl)amino)pyrimidin-4-yl)amino)-5-(5-(dimethylamino)pyrazin-2-yl)phenyl)dimethylphosphine oxide

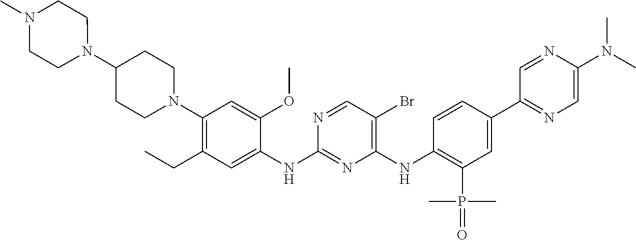

### Step 1: Synthesis of Compound 35-2

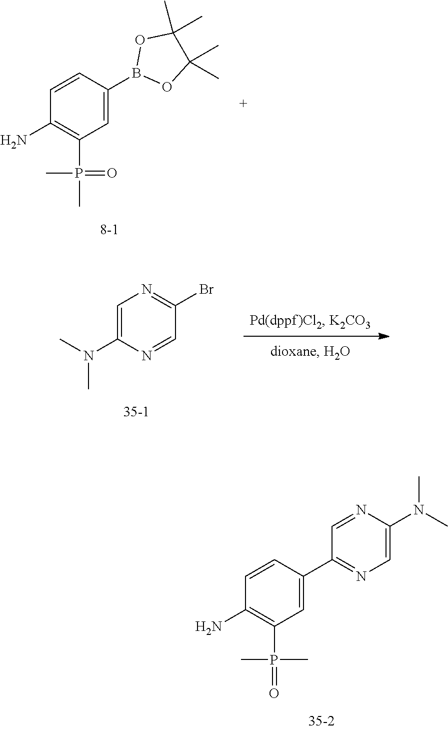

The compound 35-2 was synthesized as the method described for compound 8-3 using compound 35-1 instead of raw compound 8-2. For compound 35-2, MS: 291.13 [M+H]⁺

### Step 2: Synthesis of Compound 35-3

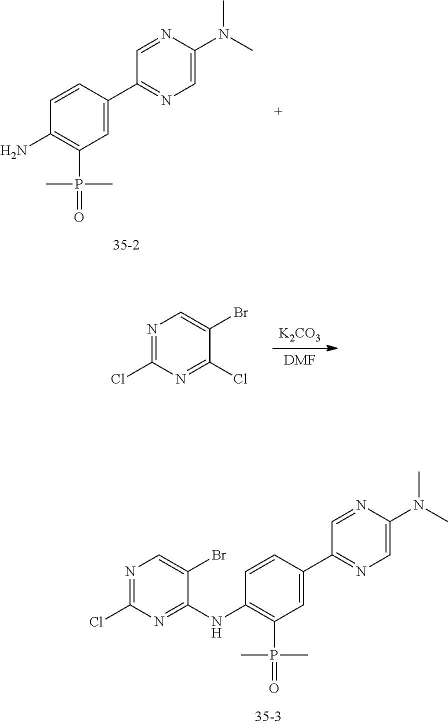

T e compound 3$-3 was synthesized as the method described tor compound 1-10 using compound 35-2 instead of raw compound 1-8. For compound 35-3, MS: 481.02 [M+H]⁺

### Step 3: Synthesis of Compound 35

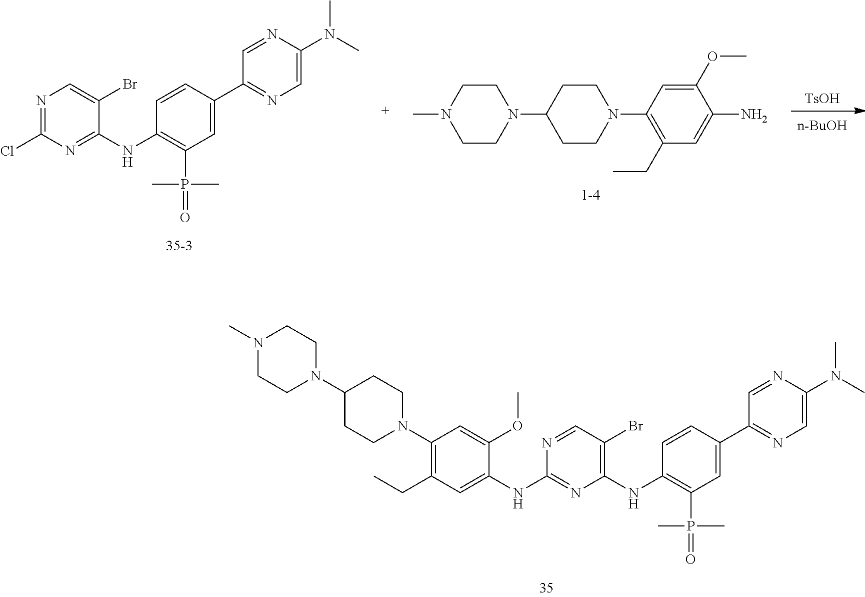

The compound 35 was synthesized as the method described for compound 1 using compound 35-3 instead of raw compound 1-10. For compound 35, MS: 777.30 [M+H]⁺

### Example 142: Synthesis of Compound 36

### (2-((5-Bromo-2-((5-ethyl-2-methoxy-4-(4-(4-methylpiperazin-1-yl)piperidin-1-yl)phen yl)amino)pyrimidin-4-yl)amino)-5-(6-cyclopropylpyridazin-3-yl)phenyl)dimethylphosphine oxide

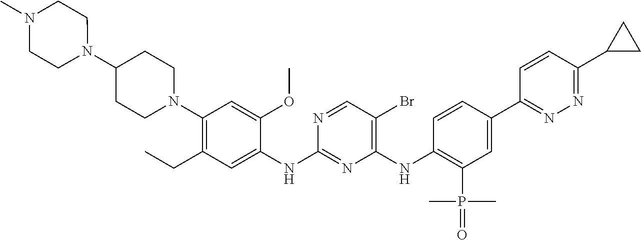 [SMILES: CCc1cc(Nc2ncc(Br)c(Nc3ccc(-c4ccc(C5CC5)nn4)cc3P(C)(C)=O)n2)c(OC)cc1N1CCC(N2CCN(C)CC2)CC1]

### Step 1: Synthesis of Compound 36-2

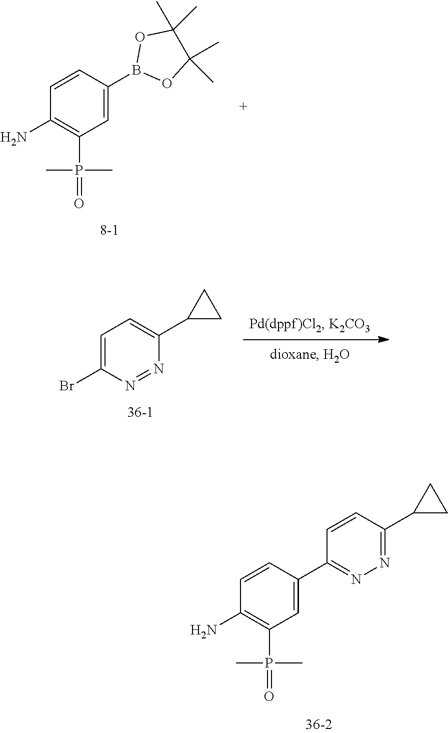 [SMILES: CC1(C)OB(c2ccc(N)c(P(C)(C)=O)c2)OC1(C)C ; Brc1ccc(C2CC2)nn1 ; CP(C)(=O)c1cc(-c2ccc(C3CC3)nn2)ccc1N]

The compound 36-2 was synthesized as the method described for compound 8-3 using compound 36-1 instead of raw compound 8-2. For compound 36-2, MS: 288.12 [M+H]⁺

### Step 2: Synthesis of Compound 36-3

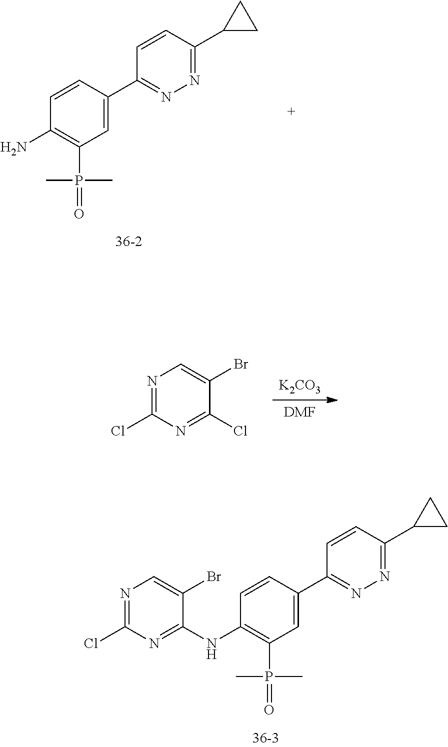

The compound 36-3 was synthesized as the method described for compound 1-10 using compound 36-2 instead of raw compound 1-8. For compound 36-3, MS: 478.01 [M+H]⁺

### Step 3: Synthesis of Compound 36

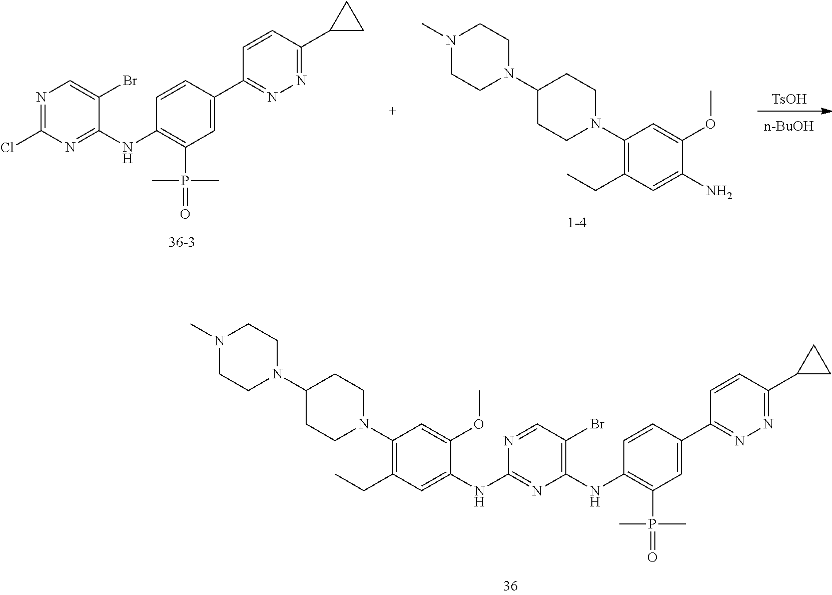

The compound 36 was synthesized as the method described for compound 1 using compound 36-3 instead of raw compound 1-10. For compound 36, MS: 774.29 [M+H]⁺

### Example 143: Synthesis of Compound 37

### (2-((5-Bromo-2-((5-ethyl-2-methoxy-4-(4-(4-methylpiperazin-1-yl)piperidin-1-yl)phenyl)amino)pyrimidin-4-yl)amino)-5-(6-(dimethylamino)pyridazin-3-yl)phenyl)dimethylphosphine oxide

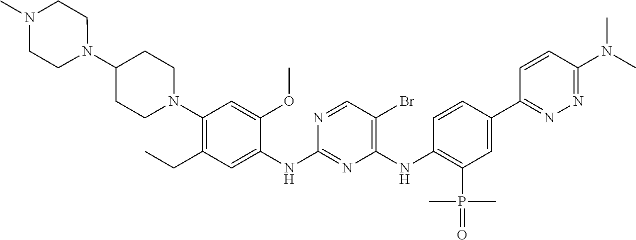

### Step 1: Synthesis of Compound 37-2

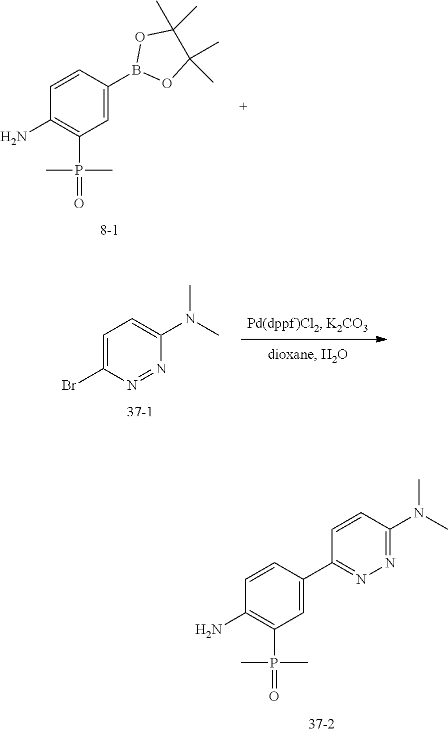

The compound 37-2 was synthesized as the method described for compound 8-3 using compound 37-1 instead of raw compound 8-2. For compound 37-2, MS: 291.13 [M+H]⁺

### Step 2: Synthesis of Compound 37-3

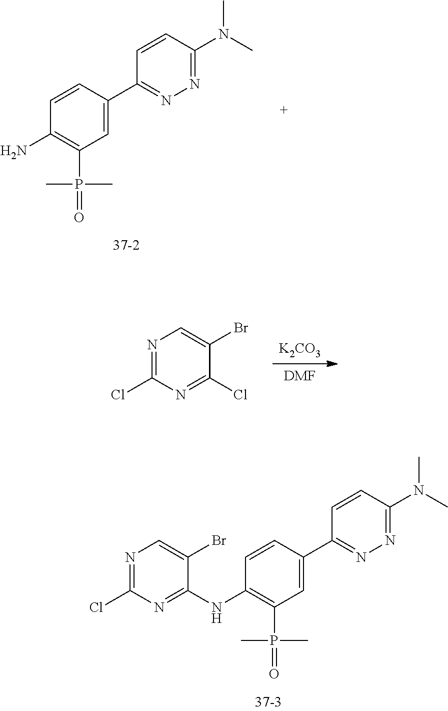

The compound 37-3 was synthesized as the method described for compound 1-10 using compound 37-2 instead of raw compound 1-8. For compound 37-3, MS: 481.02 [M+H]⁺

### Step 3: Synthesis of Compound 37

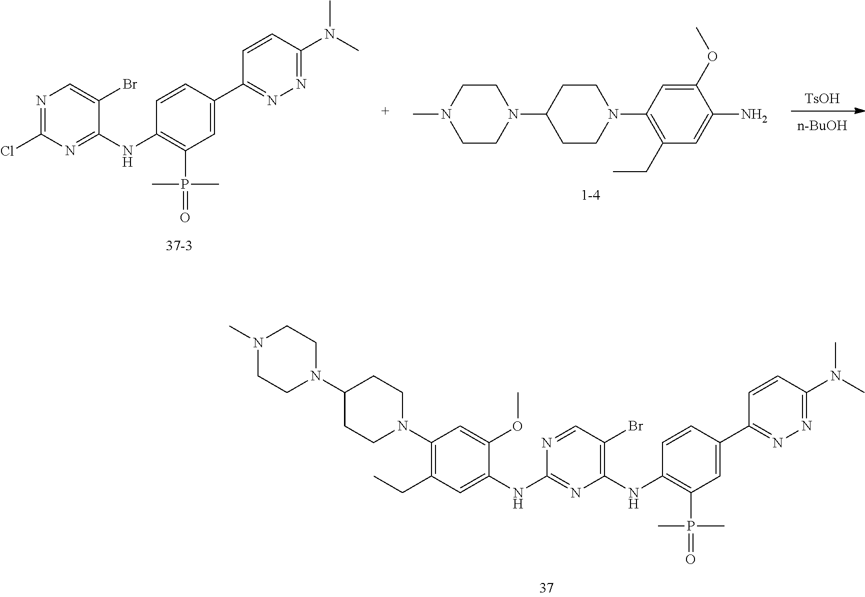

The compound 37 was synthesized as the method described for compound 1 using compound 37-3 instead of raw compound 1-10. For compound 37, MS: 777.30 [M+H]⁺

### Example 144: Synthesis of Compound 38

### (2-((5-Bromo-2-((5-ethyl-2-methoxy-4-(4-(4-methylpiperazin-1-yl)piperidin-1-yl)phenyl)amino)pyrimidin-4-yl)amino)-5-(isothiazol-4-yl)phenyl)dimethylphosphine oxide

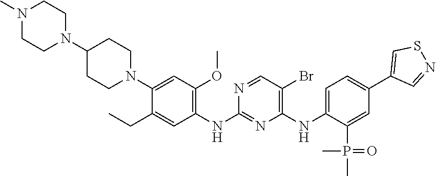 [SMILES: CCc1cc(Nc2ncc(Br)c(Nc3ccc(-c4cnsc4)cc3P(C)(C)=O)n2)c(OC)cc1N1CCC(N2CCN(C)CC2)CC1]

### Step 1: Synthesis of Compound 38-2

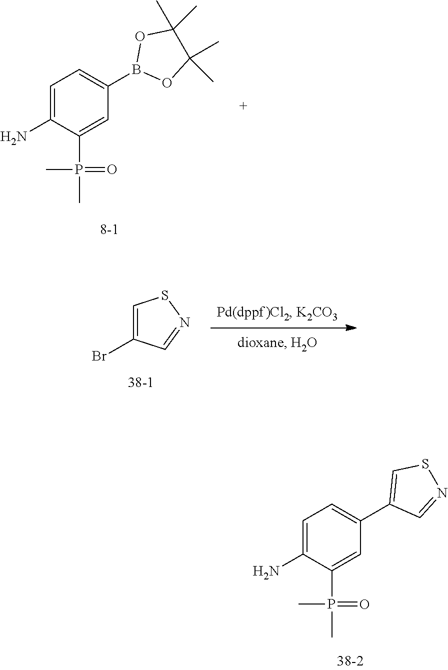 [SMILES: CC1(C)OB(c2ccc(N)c(P(C)(C)=O)c2)OC1(C)C ; Brc1cnsc1 ; CP(C)(=O)c1cc(-c2cnsc2)ccc1N]

The compound 38-2 was synthesized as the method described for compound 8-3 using compound 38-1 instead of raw compound 8-1. For compound 38-2, MS: 253.05 [M+H]⁺

### Step 2: Synthesis of Compound 38-3

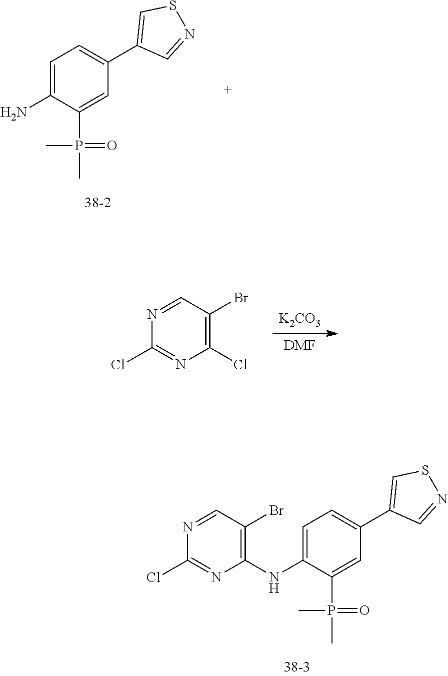

The compound 38-3 was synthesized as the method described for compound 1-10 using compound 38-2 instead of raw compound 1-8. For compound 38-3, MS: 442.94 [M+H]⁺

### Step 3: Synthesis of Compound 38

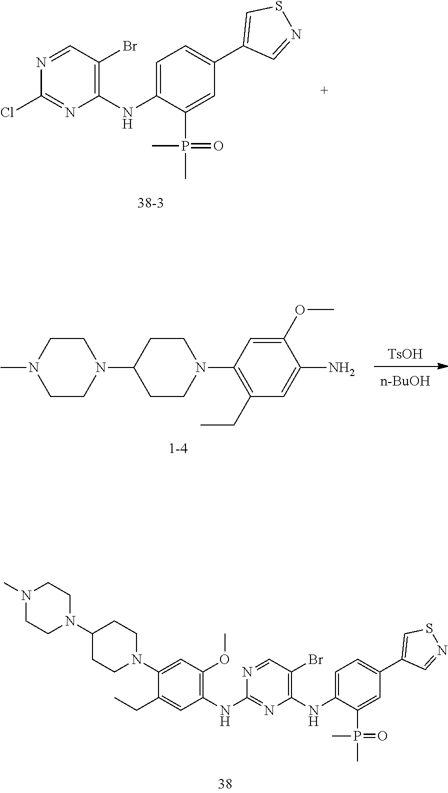

The compound 38 was synthesized as the method described for compound 1 using compound 38-3 instead of raw compound 1-10. For compound 38, MS: 739.22 [M+H]⁺

### Example 145: Synthesis of Compound 39

### (2-((5-Bromo-2-((5-ethyl-2-methoxy-4-(4-(4-methylpiperazin-1-yl)piperidin-1-yl)phenyl)amino)pyrimidin-4-yl)amino)-5-(1H-pyrazol-5-yl)phenyl)dimethylphosphine oxide

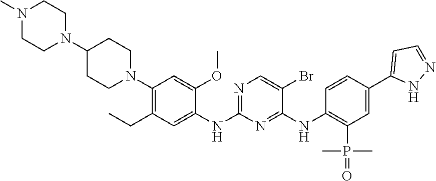 [SMILES: CCc1cc(Nc2ncc(Br)c(Nc3ccc(-c4ccn[nH]4)cc3P(C)(C)=O)n2)c(OC)cc1N1CCC(N2CCN(C)CC2)CC1]

### Step 1: Synthesis of Compound 39-2

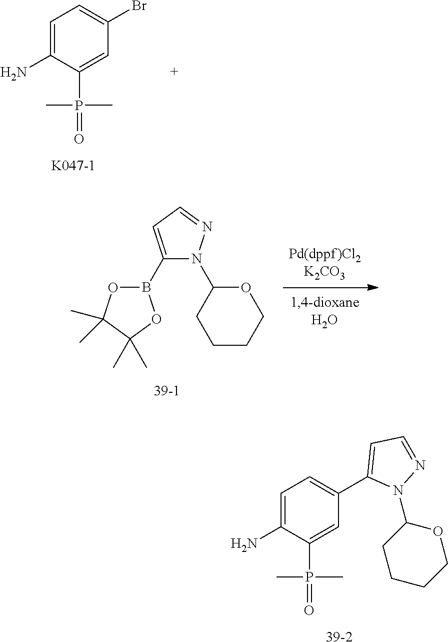

The compound 39-2 was synthesized as the method described for compound K-047-2 using compound 39-1 instead of raw compound phenylboronic acid. For compound 39-2, MS: 320.14 [M+H]⁺

### Step 2: Synthesis of Compound 39-3

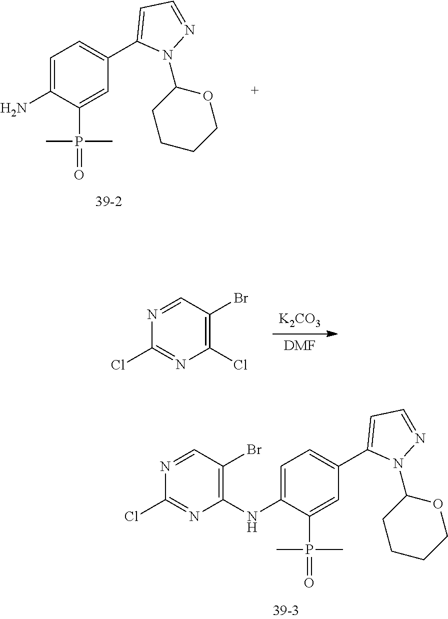

The compound 39-3 was synthesized as the method described for compound K-047-3 using compound 39-2 instead of raw compound K047-2. For compound 39-3, MS: 510.04 [M+H]⁺

### Step 3: Synthesis of Compound 39-4

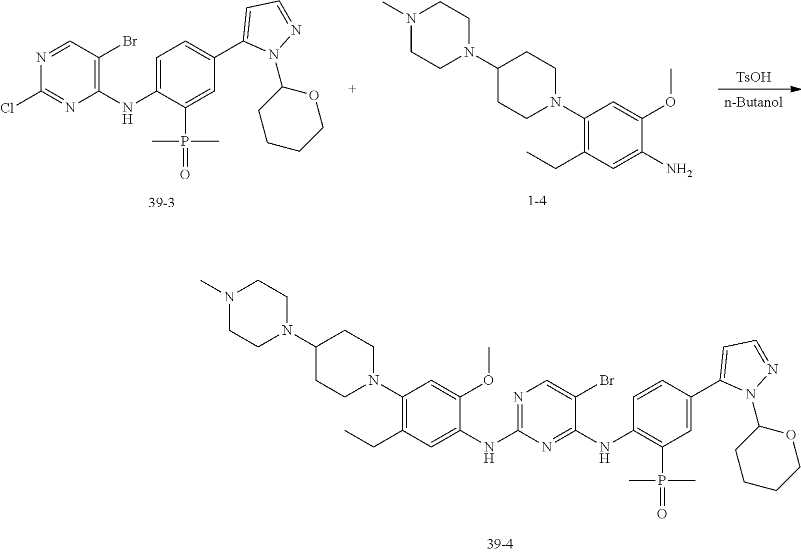

The compound 39-4 was synthesized as the method described for compound K-047 using compound 39-3 instead of raw compound K-047-3, compound 1-4 instead of raw compound K-048-4. For compound 39-4, MS: 806.32 [M+H]⁺

### Step 4: Synthesis of Compound 39

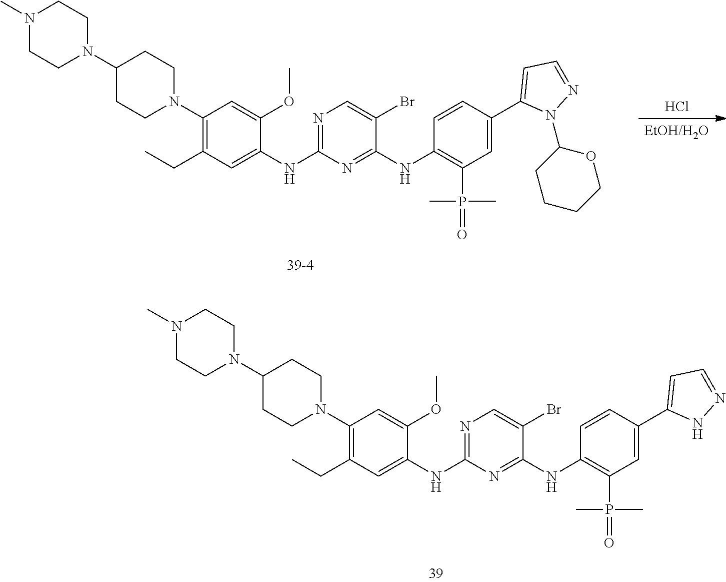

Dissolve compound 39-4 (200 mg) in ethanol (10 ml) and water (2 ml) in a 50 mL stand-up bottle, and then add concentrated hydrochloric acid (1 ml) to the solution; the reaction liquid was stirred at room temperature for 3 hr. Concentrate the reaction mixture, dissolve the residue in a small amount of methanol, dropwise add 2N aqueous sodium carbonate solution to adjust the pH to about 9; the solution was extracted twice with DCM/MeOH=10/1, combined with the organic phase, washed once with saturated sodium chloride solution, and then dried and concentrated. The residue was dissolved in dichloromethane, loaded onto a thick preparative plate and purified by scraping a large plate (DCM/MeOH=5/1); the eluent with the product was collected and concentrated to obtain compound 39 (85.2 mg), MS: 722.26 [M+H]⁺

### Example 146: Synthesis of Compound 40

### (2-((5-Choro-2-((5-ethyl-2-methoxy-4-(4-(4-methylpiperazin-1-yl)piperidin-1-yl)phenyl)amino)pyrimidin-4-yl)amino)-5-(1-methyl-1-imidazol-2-yl)phenyl)dimethylphosphine oxide

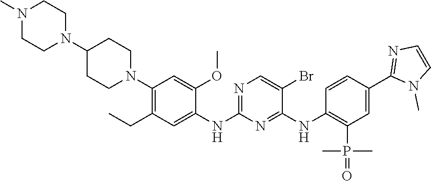 [SMILES: CCc1cc(Nc2ncc(Br)c(Nc3ccc(-c4nccn4C)cc3P(C)(C)=O)n2)c(OC)cc1N1CCC(N2CCN(C)CC2)CC1]

### Step 1: Synthesis of Compound 40

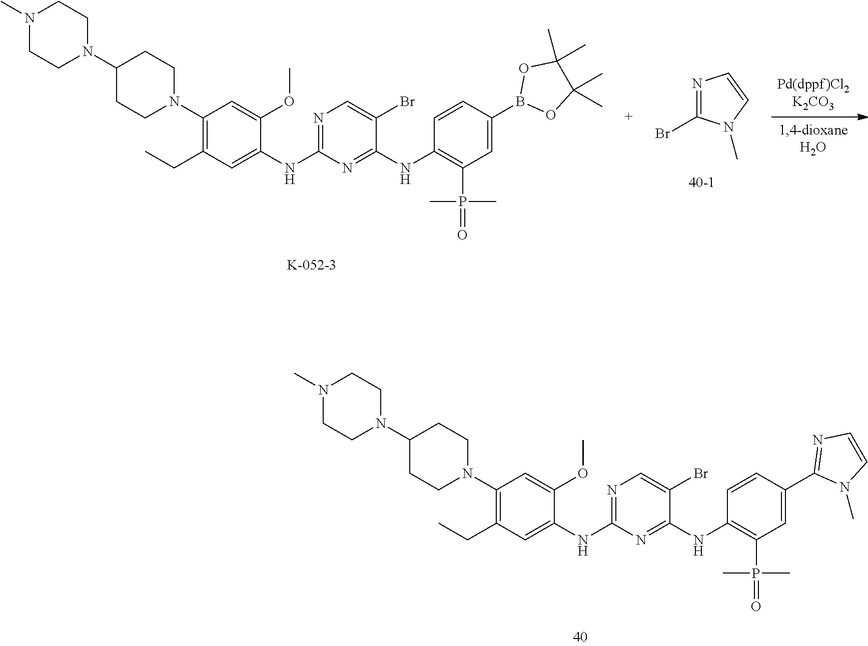

Dissolve compound K-052-3 (91 mg) in 1,4-dioxane (5 ml) and water (1 ml) in a 50 ml stand-up bottle, and then successively add 40-1 (18 mg), Pd (dppf) Cl₂ (9 mg) and anhydrous potassium carbonate (47 mg). The reaction system was replaced with nitrogen, then heated to 100° C. and stirred overnight. It was naturally cooled down to room temperature, and the solution was diluted with water (20 ml), extracted twice with dichloromethane (20 ml*2), combined with organic phase, dried over anhydrous sodium sulfate, filtered and concentrated under reduced pressure. The residue was dissolved in a small amount of dichloromethane, loaded with samples and purified on a thick preparation plate (DCM/MeOH=10/1) to obtain compound 40 (13.0 mg), MS: 692.33 [M+H]⁺

### Example 147: Synthesis of Compound 41

### (2-((5-Chloro-2-((5-ethyl-2-methoxy-4-(4-(4-methylpiperazin-1-yl)piperidin-1-yl)phenyl)amino)pyrimidin-4-yl)amino)-5-(5,5-dimethyl-5,6-dihydro-tetrahydro-pyrrolo[1,2-b]pyrazol-3-)phenyl)dimethylphosphine oxide

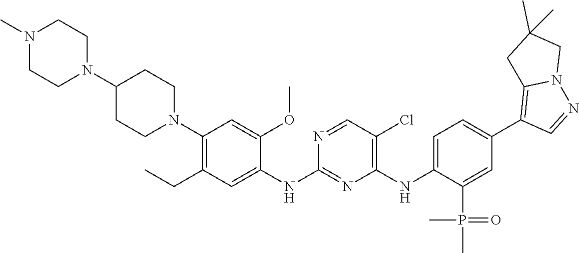 [SMILES: CCc1cc(Nc2ncc(Cl)c(Nc3ccc(-c4cnn5c4CC(C)(C)C5)cc3P(C)(C)=O)n2)c(OC)cc1N1CCC(N2CCN(C)CC2)CC1]

### Step 1: Synthesis of Compound 41

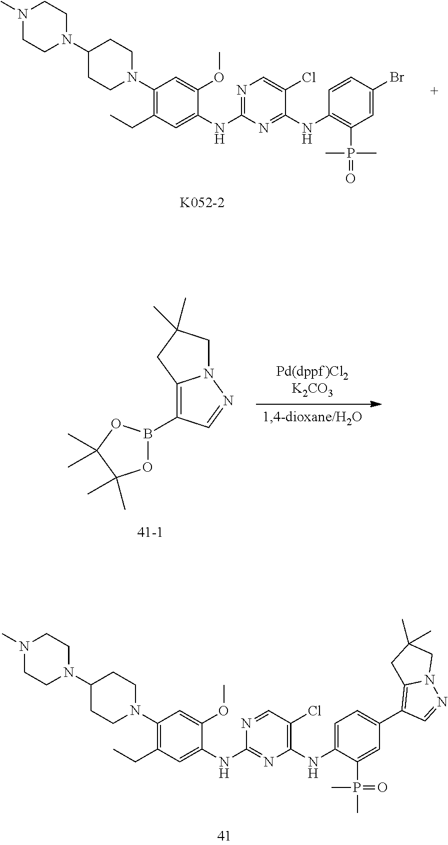

The compound 41 was synthesized as the method described for compound K-060 using compound 41-1 instead of raw compound K-060-1. For compound 41, MS: 746.37 [M+H]⁺

### Example 148: Synthesis of Compound 42

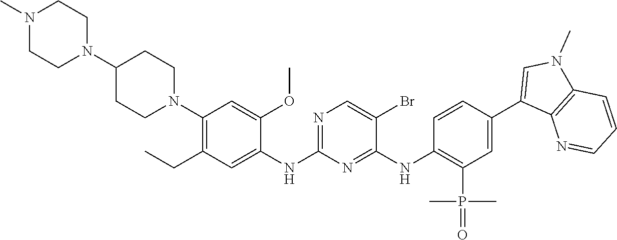 [SMILES: CCc1cc(Nc2ncc(Br)c(Nc3ccc(-c4cn(C)c5cccnc45)cc3P(C)(C)=O)n2)c(OC)cc1N1CCC(N2CCN(C)CC2)CC1]

### 2-((5-Bromo-2-((5-ethyl-2-methoxy-4-(4-(4-methylpiperazin-1-yl)piperidin-1-yl)phenyl)amino)pyrimidin-4-yl)amino)-5-(1-methyl-H-pyrrolo[3,2-b]pyridin-3-yl)phenyl)dimethylphosphine oxide

### Step 1: Synthesis of Compound 42-1

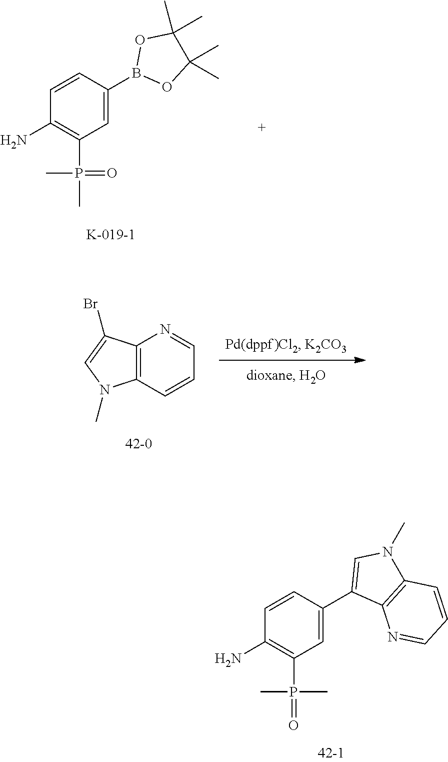 [SMILES: CC1(C)OB(c2ccc(N)c(P(C)(C)=O)c2)OC1(C)C ; Cn1cc(Br)c2ncccc21 ; Cn1cc(-c2ccc(N)c(P(C)(C)=O)c2)c2ncccc21]

The compound 42-1 was synthesized as the method described for compound K-019-2 using compound 42-0 instead of raw compound 2-Bromothiazole. For compound 42-1, MS: 300.12 [M+H]⁺

### Step 2: Synthesis of Compound 42-2

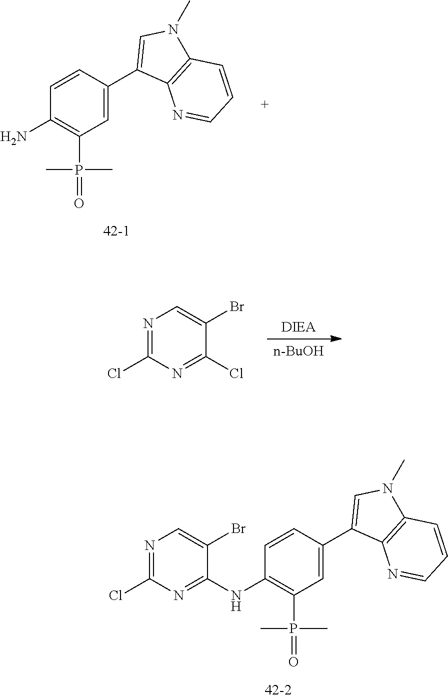

The compound 42-2 was synthesized as the method described for compound K-019-3 using compound 42-0 instead of raw compound K-019-2. For compound 42-2, MS: 490.0 M [M+H]⁺

### Step 3: Synthesis of Compound 42

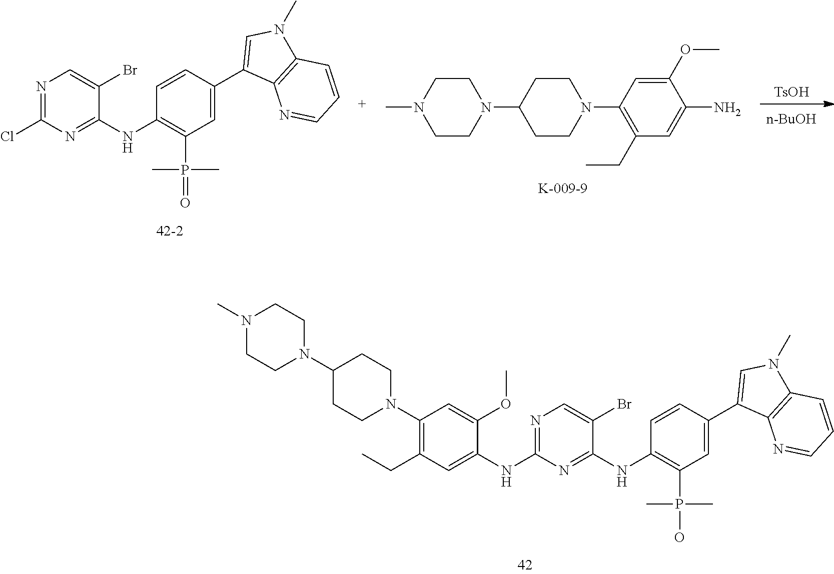

The compound 42 was synthesized as the method described for compound K-018 using compound K-009-9 instead of raw compound K-018-9, compound 42-2 instead of raw compound K-018-5. For compound 42, MS: 786.29 [M+H]⁺

### Example 149: Synthesis of Compound 43

### 2-(4-((5-Bromo-2-((5-ethyl-2-methoxy-4-(4-(4-methylpiperazin-1-yl)piperidin-1-yl)phenyl)amino)pyrimidin-4-yl)amino)-3-(dimethylphosphino)phenyl)-1H-indole-5-carbonitrile

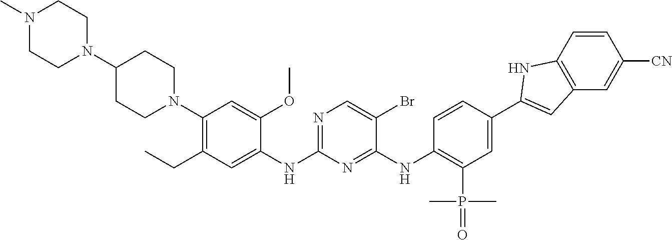 [SMILES: CCc1cc(Nc2ncc(Br)c(Nc3ccc(-c4cc5cc(C#N)ccc5[nH]4)cc3P(C)(C)=O)n2)c(OC)cc1N1CCC(N2CCN(C)CC2)CC1]

### Step 1: Synthesis of Compound 43-1

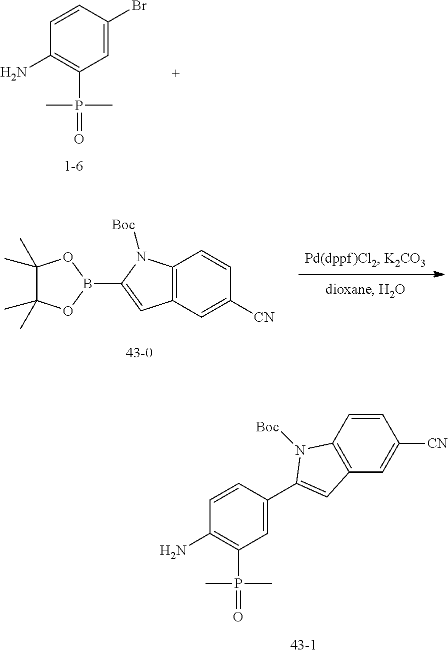 [SMILES: CP(C)(=O)c1cc(Br)ccc1N ; CC(C)(C)OC(=O)n1c(B2OC(C)(C)C(C)(C)O2)cc2cc(C#N)ccc21 ; CC(C)(C)OC(=O)n1c(-c2ccc(N)c(P(C)(C)=O)c2)cc2cc(C#N)ccc21]

The compound 43-1 was synthesized as the method described for compound 1-8 using compound 43-0 instead of raw compound 1-12. For compound 43-1, MS: 410.16 [M+H]⁺

### Step 2: Synthesis of Compound 43-2

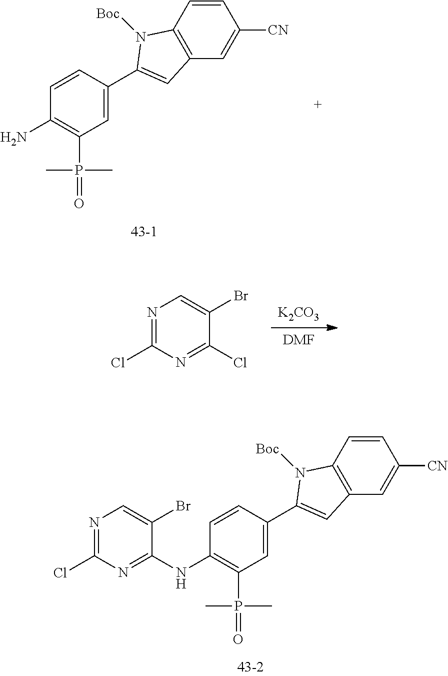

The compound 43-2 was synthesized as the method described for compound 1-10 using compound 43-1 instead of raw compound 1-8. For compound 43-2, MS: 600.05 [M+H]⁺

### Step 3: Synthesis of Compound 43

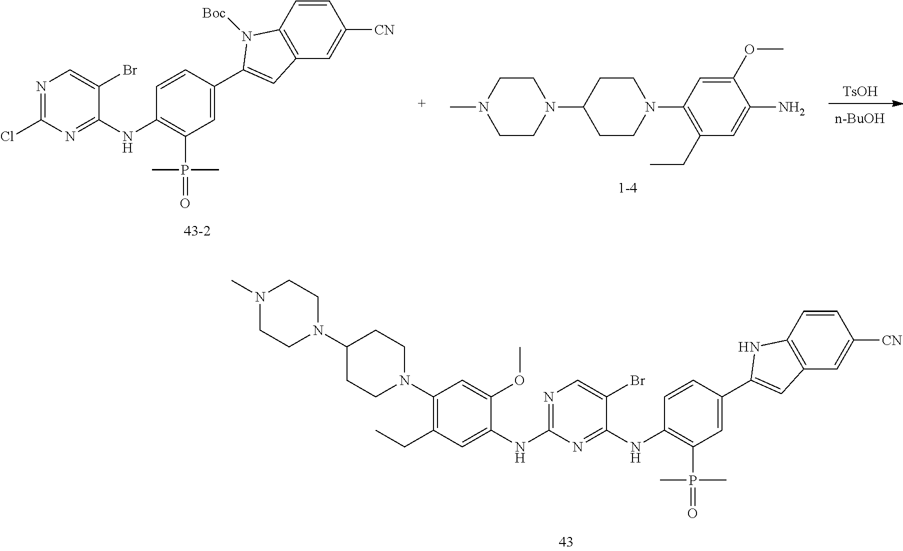

The compound 43 was synthesized as the method described for compound 1 using compound 43-2 instead of raw compound 1-10. For compound 43, MS: 796.28 [M+H]⁺

### Example 150: Synthesis of Compound 44

### (2-((5-Bromo-2-((5-ethyl-2-methoxy-4-(4-(4-methylpiperazin-1-yl)piperidin-1-yl)phenyl)amino)pyrimidin-4-yl)amino)-5-(4-fluoro-1H-pyrazol-1-yl)phenyl)dimethylphosphine oxide

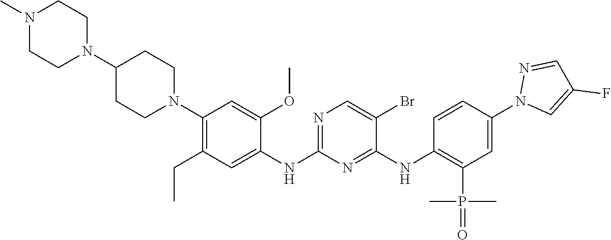 [SMILES: CCc1cc(Nc2ncc(Br)c(Nc3ccc(-n4cc(F)cn4)cc3P(C)(C)=O)n2)c(OC)cc1N1CCC(N2CCN(C)CC2)CC1]

### Step 1: Synthesis of Compound 44-1

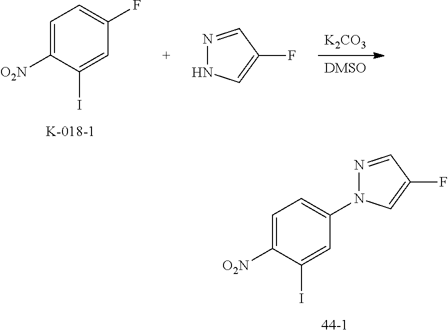

The compound 44-1 was synthesized as the method described for compound K-018-2 using compound 4-Fluoro-1H-pyrazole instead of raw compound 4-Methoxy-1H-pyrazole. For compound 44-1, MS: 333.94 [M+H]⁺

### Step 2: Synthesis of Compound 44-2

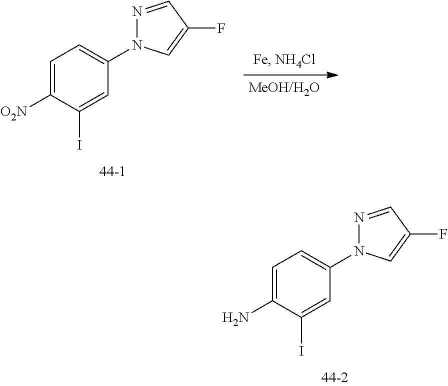

The compound 44-2 was synthesized as the method described for compound K-018-3 using compound 44-1 instead of raw compound K-018-2. For compound 44-2, MS: 303.97 [M+H]⁺

### Step 3: Synthesis of Compound 44-3

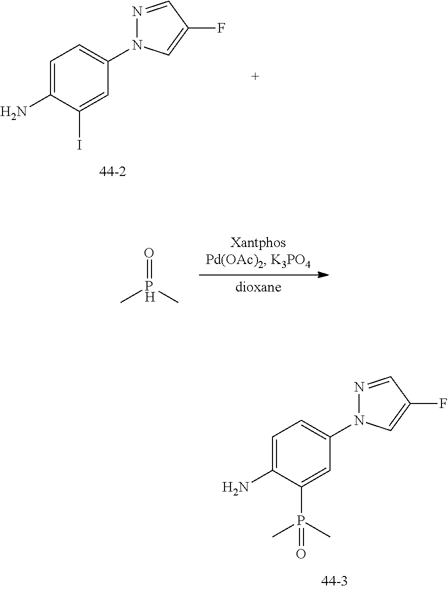

The compound 44-3 was synthesized as the method described for compound K-018-4 using compound 44-2 instead of raw compound K-018-3. For compound 44-3, MS: 254.08 [M+H]⁺

### Step 4: Synthesis of Compound 44-4

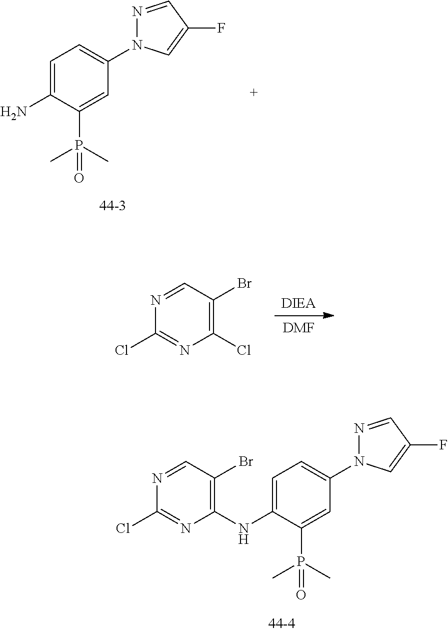

The compound 44-4 was synthesized as the method described for compound K-018-5 using compound 44-3 instead of raw compound K-018-4. For compound 44-4, MS: 443.97 [M+H]⁺

### Step 5: Synthesis of Compound 44

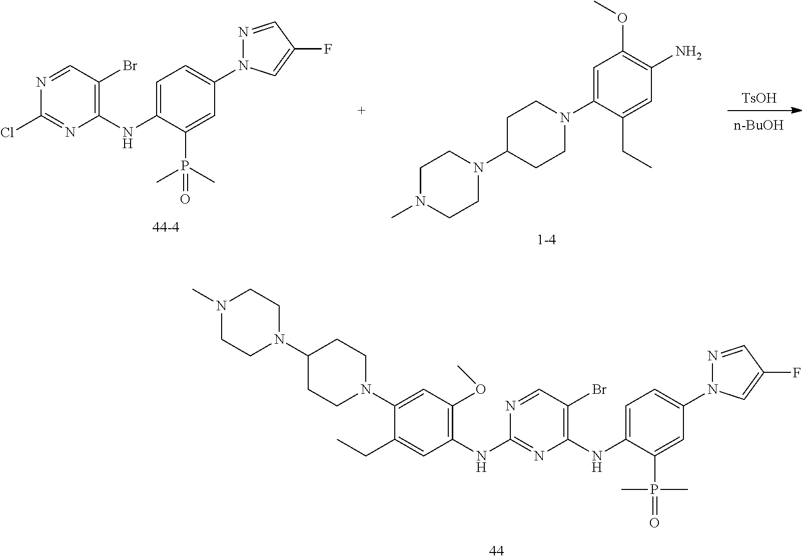

The compound 44 was synthesized as the method described for compound 1 using compound 44-4 instead of raw compound 1-10. For compound 44, MS: 740.25 [M+H]⁺

### Example 151: Synthesis of Compound 45

### (2-((5-Chloro-2-((5-ethyl-2-methoxy-4-(4-(4-methylpiperazin-1-yl)piperidin-1-yl)phenyl)amino)pyrimidin-4-yl)amino)-5-(5-methyl-1,3,4-oxadiazol-2-yl)phenyl)dimethylphosphine oxide

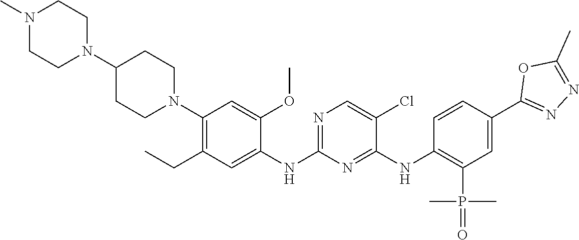 [SMILES: CCc1cc(Nc2ncc(Cl)c(Nc3ccc(-c4nnc(C)o4)cc3P(C)(C)=O)n2)c(OC)cc1N1CCC(N2CCN(C)CC2)CC1]

### Step 1: Synthesis of Compound 45

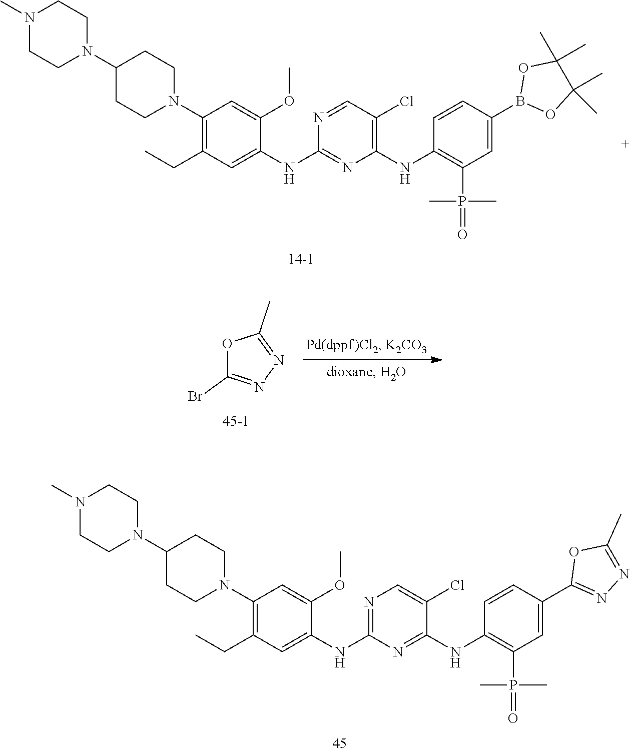

Add compounds 14-1 (100 mg), 45-1 (40 mg), Pd(dppf)Cl₂ (12 mg), K₂CO₃ (40 mg), dioxane (4 ml) and water (1 mL) sequentially to the reaction flask, heat to 100° C. under nitrogen protection, and stir with heating for 12 hr. The reaction was complete as monitored by LCMS, and then stopped. Remove the solvent, separated and purified by preparative TLC (dichloromethane:methanol=10:1), to obtain the off-white solid of desired compound 45 (20 mg), MS: 694.22 [M+H]⁺

### Example 152: Synthesis of Compound 46

### (2-((5-Bromo-2-((5-ethyl-2-methoxy-4-(-4-(4-methylpiperazin-1-yl)piperidin-1-yl)phenyl)amino)pyrimidin-4-yl)amino)-5-(1-methyl-1H-pyrrolo[2,3-b]pyridin-3-yl)phenyl)dimethylphosphine oxide

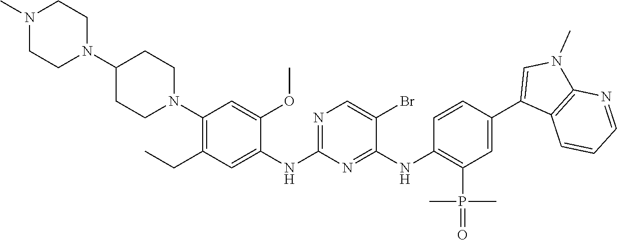 [SMILES: CCc1cc(Nc2ncc(Br)c(Nc3ccc(-c4cn(C)c5ncccc45)cc3P(C)(C)=O)n2)c(OC)cc1N1CCC(N2CCN(C)CC2)CC1]

### Step 1: Synthesis of Compound 46-1

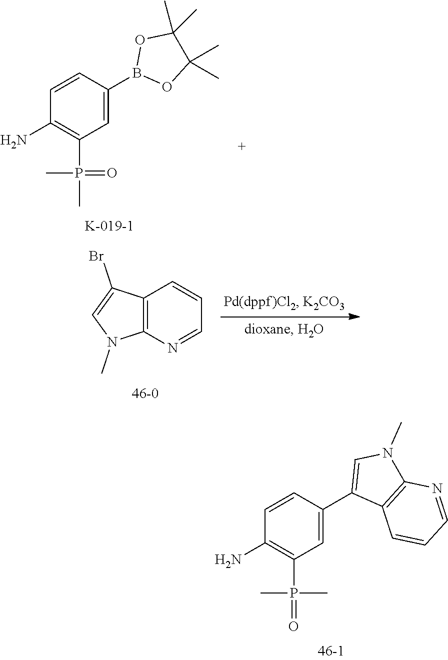 [SMILES: CC1(C)OB(c2ccc(N)c(P(C)(C)=O)c2)OC1(C)C ; Cn1cc(Br)c2cccnc21 ; Cn1cc(-c2ccc(N)c(P(C)(C)=O)c2)c2cccnc21]

The compound 46-1 was synthesized as the method described for compound K-019-2 using compound 46-0 instead of raw compound 2-Bromothiazole. For compound 46-1, MS: 300.12 [M+H]⁺

### Step 2: Synthesis of Compound 46-2

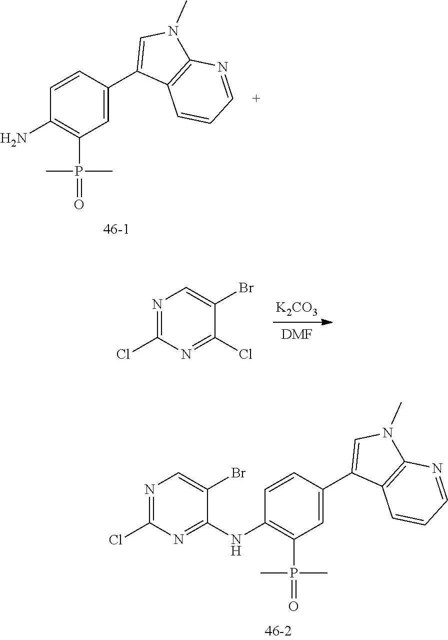

The compound 46-2 was synthesized as the method described for compound K-019-3 using compound 46-1 instead of raw compound K-019-2. For compound 46-2, MS: 491 [M+H]⁺

### Step 3: Synthesis of Compound 46

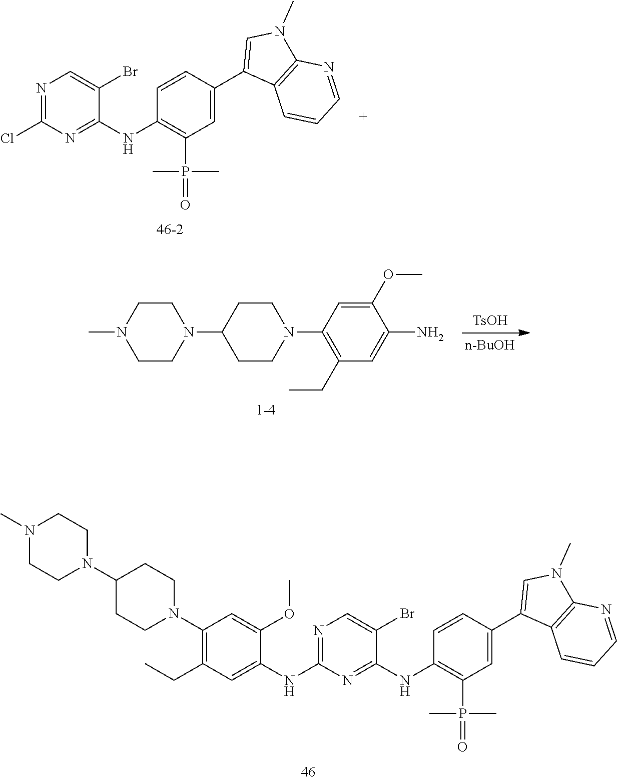

The compound 46 was synthesized as the method described for compound K-018 using compound 1-4 instead of raw compound K-018-9, compound 46-2 instead of raw compound K-018-5. For compound 46, MS: 786.29 [M+H]⁺

¹H NMR (500 MHz, DMSO-d₆) δ 11.91 (s, 1H), 9.85 (s, 1H), 8.44-8.32 (m, 3H), 8.19-8.11 (m, 2H), 7.90-7.87 (m, 1H), 7.63 (s, 1H), 7.40-7.31 (m, 2H), 6.89 (s, 1H), 3.94 (s, 3H), 3.81 (s, 3H), 3.74-3.73 (m, 4H), 3.57-3.56 (m, 4H), 3.29 (s, 1H), 3.17-3.11 (m, 21H), 2.51-2.50 (m, 6H), 2.85-2.77 (m, 5H), 2.08-2.07 (m, 2H), 1.96-1.94 (m, 7H).

### Example 153: Synthesis of Compound 47

### (2-((5-Bromo-2-((5-ethyl-2-methoxy-4-(4-(4-methylpiperazin-1-yl)piperidin-1-yl)phenyl)amino)pyrimidin-4-yl)amino)-5-(1-(2-morpholinoethyl)-1hydro-pyrazol-4-yl)phenyl)dimethylphosphine oxide

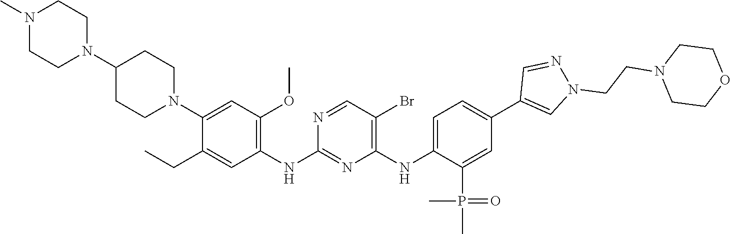 [SMILES: CCc1cc(Nc2ncc(Br)c(Nc3ccc(-c4cnn(CCN5CCOCC5)c4)cc3P(C)(C)=O)n2)c(OC)cc1N1CCC(N2CCN(C)CC2)CC1]

### Step 1: Synthesis of Compound 47-2

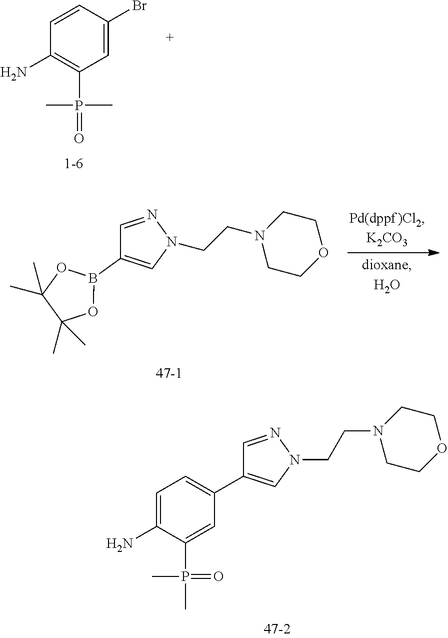 [SMILES: CP(C)(=O)c1cc(Br)ccc1N ; CC1(C)OB(c2cnn(CCN3CCOCC3)c2)OC1(C)C ; CP(C)(=O)c1cc(-c2cnn(CCN3CCOCC3)c2)ccc1N]

The compound 47-2 was synthesized as the method described for compound 1-8 using compound 47-1 instead of raw compound 1-7. For compound 47-2, MS: 349.17 [M+H]⁺

### Step 2: Synthesis of Compound 47-3

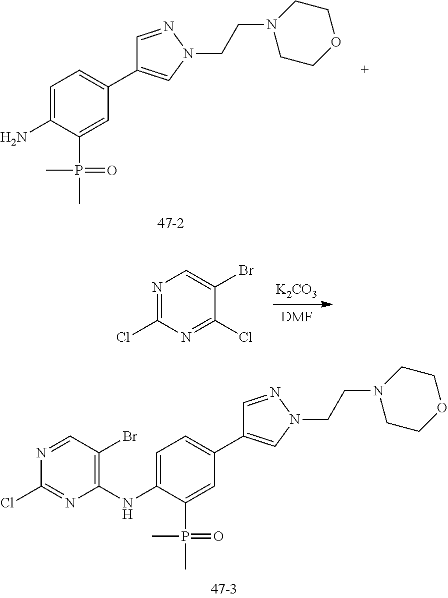

The compound 47-3 was synthesized as the method described for compound 1-10 using compound 47-2 instead of raw compound 1-8. For compound 47-3, MS: 539.06 [M+H]⁺

### Step 3: Synthesis of Compound 47

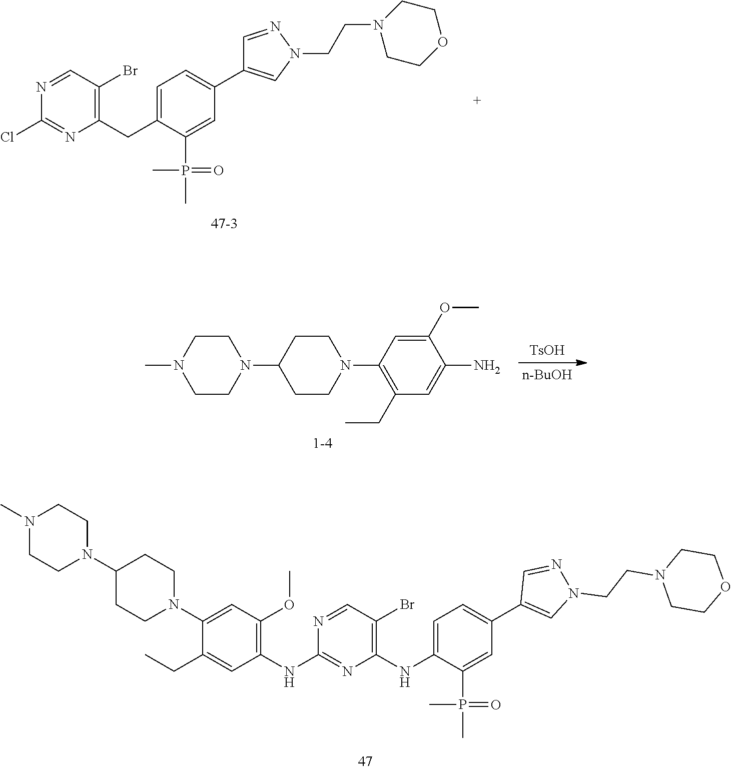

The compound 47 was synthesized as the method described for compound 1 using compound 47-3 instead of raw compound 1-10. For compound 47, MS: 835.35 [M+H]⁺

### Example 154: Synthesis of Compound 48

### (2-((5-Chloro-2-((5-ethyl-2-methoxy-4-(4-(4-methylpiperazin-1-yl)piperidin-1-yl)phenyl)amino)pyrimidin-4-yl)amino)-5-(1H-pyrazol-4-yl)phenyl)dimethylphosphine oxide

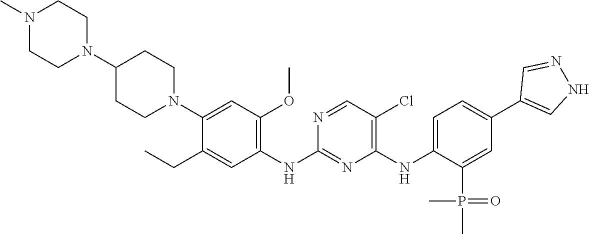 [SMILES: CCc1cc(Nc2ncc(Cl)c(Nc3ccc(-c4cn[nH]c4)cc3P(C)(C)=O)n2)c(OC)cc1N1CCC(N2CCN(C)CC2)CC1]

### Step 1: Synthesis of Compound 48-2

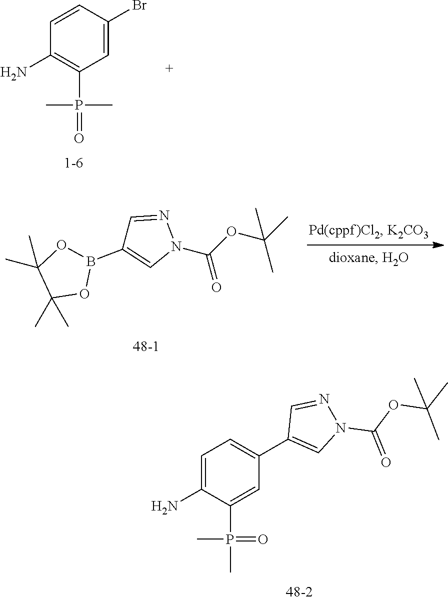 [SMILES: CP(C)(=O)c1cc(Br)ccc1N ; CC(C)(C)OC(=O)n1cc(B2OC(C)(C)C(C)(C)O2)cn1 ; CC(C)(C)OC(=O)n1cc(-c2ccc(N)c(P(C)(C)=O)c2)cn1]

The compound 48-2 was synthesized as the method described for compound 1-8 using compound 48-1 instead of raw compound 1-7. For compound 48-2, MS: 336.14 [M+H]⁺

### Step 2: Synthesis of Compound 48-4

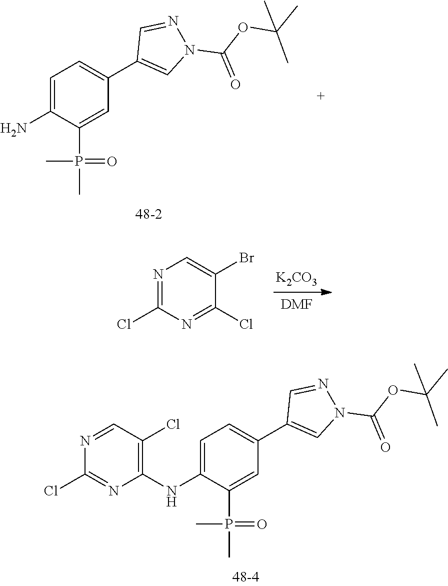

The compound 48-4 was synthesized as the method described for compound 1-10 using compound 48-2 instead of raw compound 1-8, compound 48-3 instead of raw compound 5-Bromo-2,4-dichloropyrimidine. For compound 48-4, MS: 482.08 [M+H]⁺

### Step 3: Synthesis of Compound 48

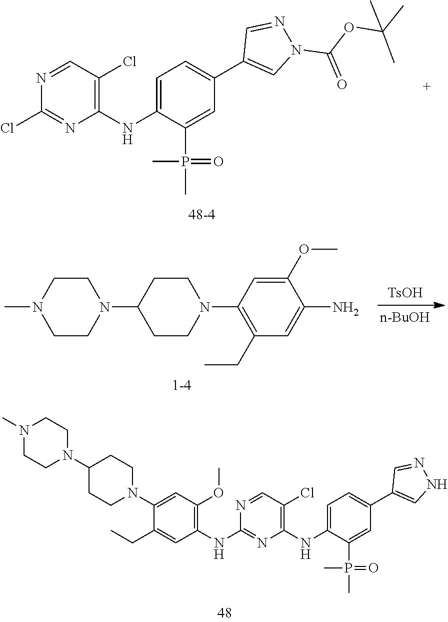

The compound 48 was synthesized as the method described for compound P using compound 48-4 instead of raw compound 1-10. For compound 48, MS: 678.31 [M+H]⁺

### Example 155: Synthesis of Compound 49

### (2-((5-Bromo-2-((5-ethyl-2-methoxy-4-(4-(4-methylpiperazin-1-yl)piperidin-1-yl)phenyl)amino)pyrimidin-4-yl)amino)-5-(1H-pyrazol-1-yl)phenyl)dimethylphosphine oxide

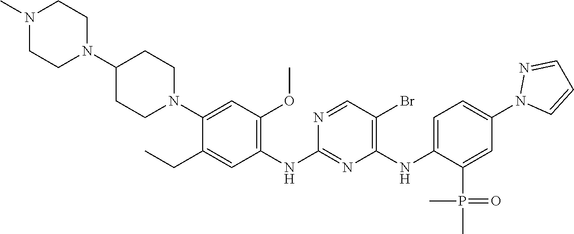 [SMILES: CCc1cc(Nc2ncc(Br)c(Nc3ccc(-n4cccn4)cc3P(C)(C)=O)n2)c(OC)cc1N1CCC(N2CCN(C)CC2)CC1]

### Step 1: Synthesis of Compound 49

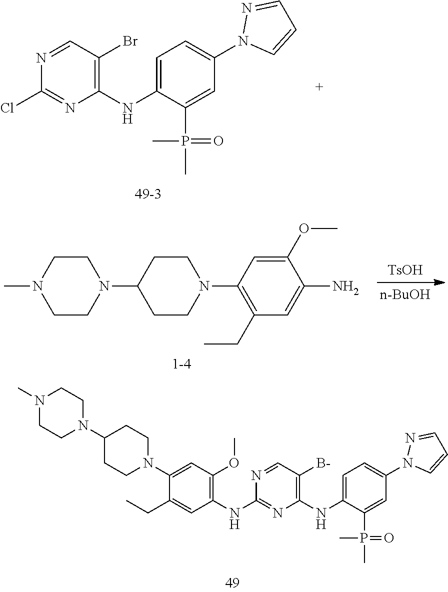

The compound 49 was synthesized as the method described for compound 1 using compound 49-3 instead of raw compound 1-10. For compound 49, MS: 722.26 [M+H]⁺

### Example 156: Synthesis of Compound 50

### (2-((5-Bromo-2-((5-ethyl-2-methoxy-4-(4-(4-methylpiperazin-1-yl)piperidin-1-yl)phenyl)amino]amino]pyrimidin-4-yl)amino)-5-(4-methoxy-1H-pyrazol-1-yl)phenyl)dimethylphosphine oxide

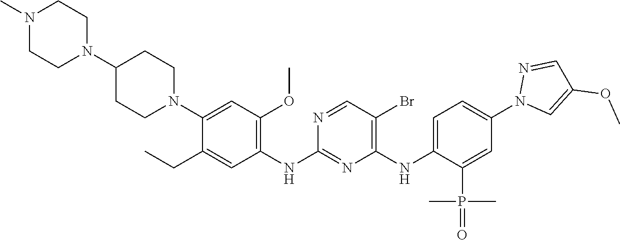

The compounds 50-2, 50-3, 50-4 and 50-5 were synthesized as the procedure described for compound K-089, except that the reaction raw materials were replaced. The raw materials used in this example were the intermediates or commercially available compounds synthesized in other examples of the invention,

### Step 1: Synthesis of Compound 50-8

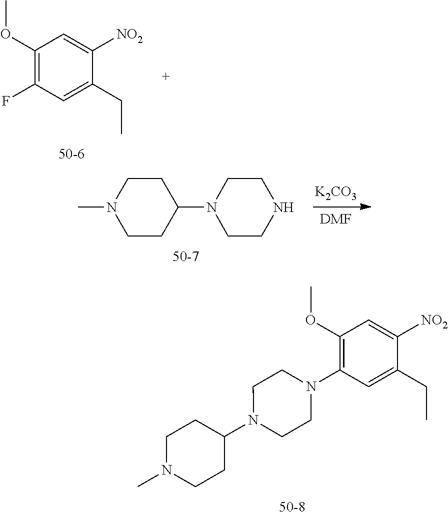

Add 50-6, 50-7, K₂CO₃ and DMF sequentially to the reaction flask, heat to 90° C., and stir with heating for 12 hr. The reaction was complete as monitored by LCMS, and then stopped. The reaction solution was poured into water (100 mL), filtered with suction, and the filter cake was washed with water and dried to obtain yellow solid of desired product 50-8, MS: 363.24 [M+H]⁺

### Step 6: Synthesis of Compound 50-9

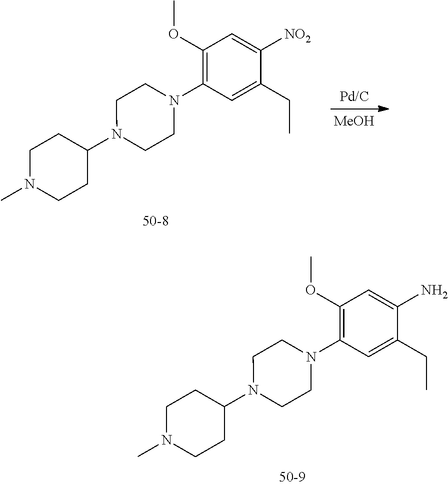 [SMILES: "MeOH" ; CCc1cc(N2CCN(C3CCN(C)CC3)CC2)c(OC)cc1[N+](=O)[O-] ; CCc1cc(N2CCN(C3CCN(C)CC3)CC2)c(OC)cc1N]

Add 50-8, Pd/C (10%) and MeOH to the reaction flask, and introduce H₂, the reaction solution was stirred at room temperature for 3 hr. The reaction was complete as monitored by LCMS, and then stopped. The solution was filtered with suction, and rinsed with methanol (20 mL); collect the organic phase, move the solvent, and obtain the reddish-brown liquid of desired product compound 50-9, MS: 333.26 [M+H]⁺

### Step 7: Synthesis of Compound 50

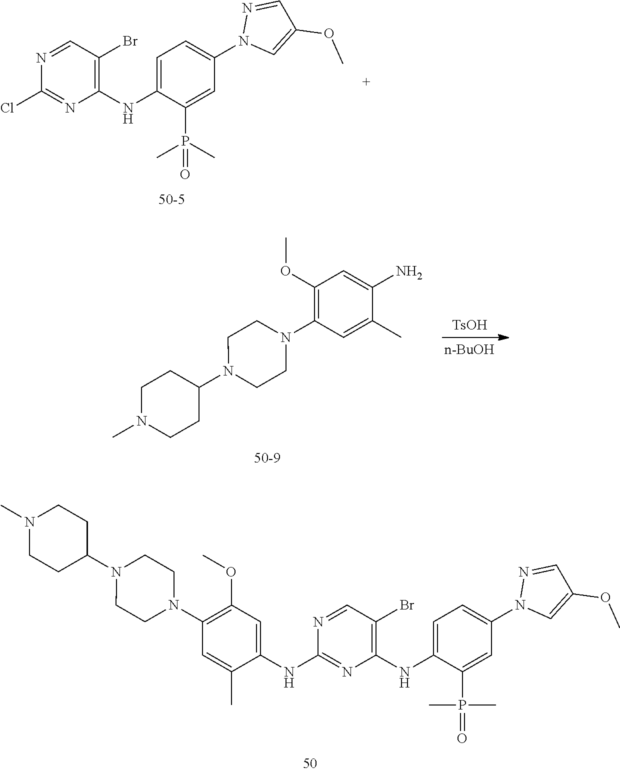

Add compounds 50-5, 50-9, p-toluenesulfonic acid and n-butanol sequentially to the reaction flask, heat to 120° C., and stir for 12 hr. The reaction was complete as monitored by LCMS, and then stopped. The reaction solution was poured into sodium carbonate solution (5 mL), and extracted with dichloromethane (3×50 mL); the organic phase was washed with saturated brine solution (3×30 mL), dried over anhydrous sodium sulfate, separated and purified by column chromatography (dichloromethane:methanol=12:1), and then concentrated to obtain the off-white solid of compound 50, MS: 738.26 [M+H]⁺

### Example 157: Synthesis of Compound 51

### 4′-((5-Bromo-2-((5-ethyl-2-methoxy-4-(4-(4-methylpiperazin-1-yl)piperidin-1-yl)phenyl)amino)pyrimidin-4-yl)amino)-3′-(dimethylphosphoryl)-[1,1′-biphenyl]-2-carbonitrile

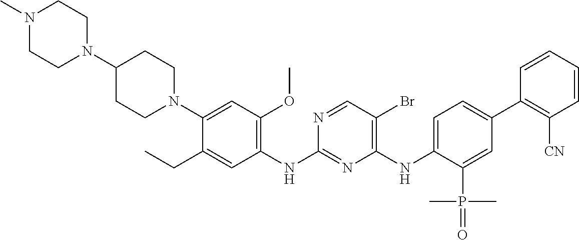 [SMILES: CCc1cc(Nc2ncc(Br)c(Nc3ccc(-c4ccccc4C#N)cc3P(C)(C)=O)n2)c(OC)cc1N1CCC(N2CCN(C)CC2)CC1]

### Step 1: Synthesis of Compound 51-3

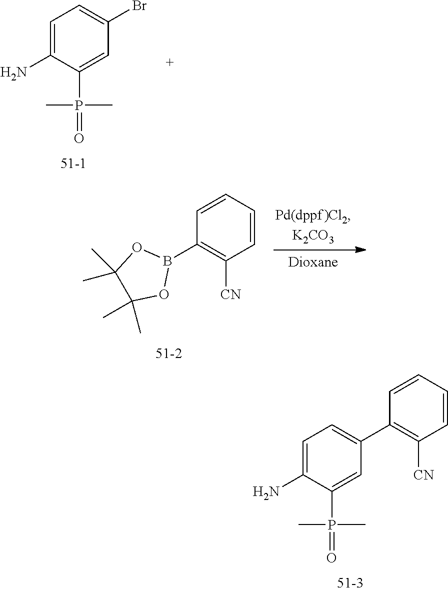 [SMILES: CP(C)(=O)c1cc(Br)ccc1N ; CC1(C)OB(c2ccccc2C#N)OC1(C)C ; CP(C)(=O)c1cc(-c2ccccc2C#N)ccc1N]

Add compounds 51-1, 51-2. Pd(dppf)Cl₂, K₂CO₃, dioxane and water to the reaction flask, heat to 90° C. under nitrogen protection, and stir with heating for 16 hr. The reaction was complete as monitored by LCMS, and then stopped. The reaction mixture was poured into water (50 mL), and extracted with dichloromethane (3×50 mL); the organic phase was washed with saturated brine (3×30 mL), dried over anhydrous sodium sulfate, separated and purified by column chromatography (dichloromethane:methanol=15:1), concentrated and dried to obtain the yellow solid of desired product 51-3, MS: 271.09 [M+H]⁺

### Step 2: Synthesis of Compound 51-5

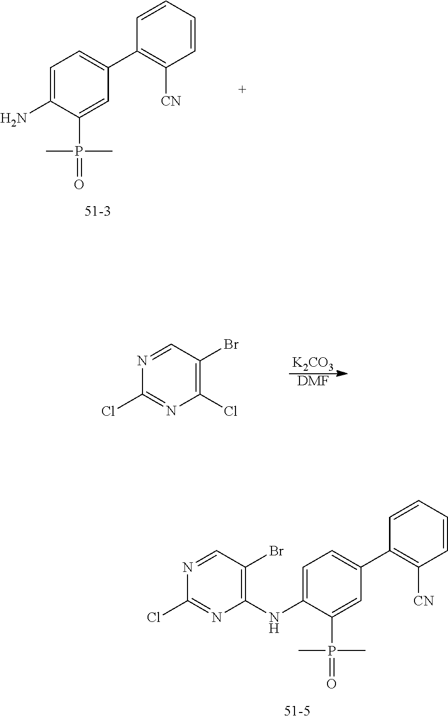

Dissolve 51-3, 5-Bromo-2,4-dichloropyrimidine to DMF in a reactor equipped with a stir bar and nitrogen protector, add K₂CO₃ at room temperature, and the gas in reactor was replaced with nitrogen for three times. After well stirring, the solution was transferred to an oil bath, heated to 100° C., and stirred at 100° C. for 16 hr. The reaction was complete as monitored by LCMS. The reaction solution was cooled down to room temperature, poured into water, and extracted with ethyl acetate (30 ml) for three times; the organic phase was concentrated by rotary evaporator. Silica gel was added to the concentrated organic phase to mix the sample, and then which was separated and purified by PE:EtOAc column with gradient from 0% to 80%. After that, it was concentrated and dried to obtain the compound 51-5, MS: 460.99 [M+H]⁺

### Step 3: Synthesis of Compound SI

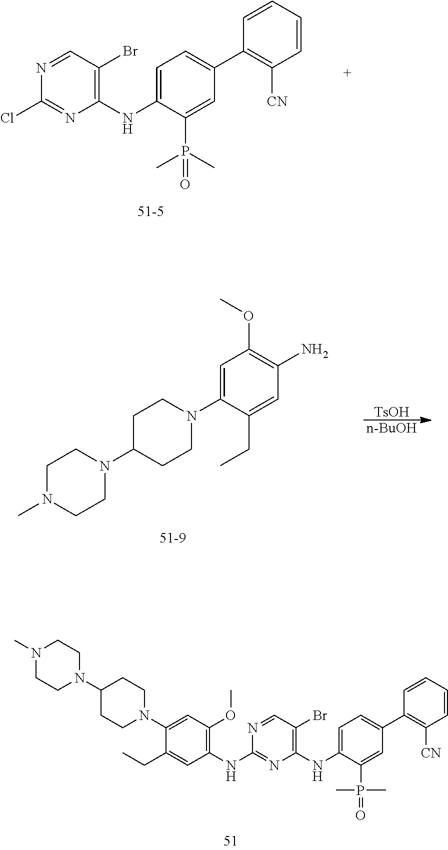

Add compounds 51-5, 5-Bromo-2,4-dichloropyrimidine, p-toluenesulfonic acid and n-butanol to the reaction flask, heat to 120° C., and stir for 12 hr. The reaction was complete as monitored by LCMS, and then stopped. The reaction solution was poured into sodium carbonate solution (5 mL), and extracted with dichloromethane (3×50 mL); the organic phase was washed with saturated brine (3×30 mL), dried over anhydrous sodium sulfate, separated and purified by column chromatography (dichloromethane:methanol=12:1), and then concentrated to obtain off-white solid of compound 51, MS: 757.27 [M+H]⁺

### Example 158: Synthesis of Compound 52

### (2-((5-Bromo-2-((5-ethyl-2-methoxy-4-(4-(4-methylpiperazin-1-yl)piperidin-1-yl)phenyl)amino)amino]pyrimidin-4-yl)amino)-5-(1H-Indol-7-yl)phenyl)dimethylphosphine oxide

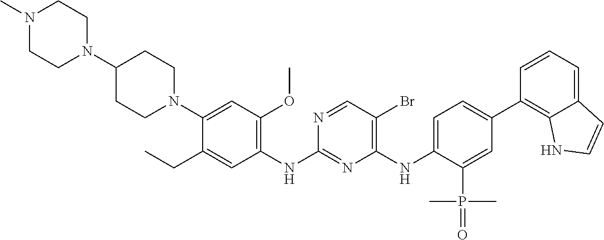 [SMILES: CCc1cc(Nc2ncc(Br)c(Nc3ccc(-c4cccc5cc[nH]c45)cc3P(C)(C)=O)n2)c(OC)cc1N1CCC(N2CCN(C)CC2)CC1]

### Step 1: Synthesis of Compound 52-3

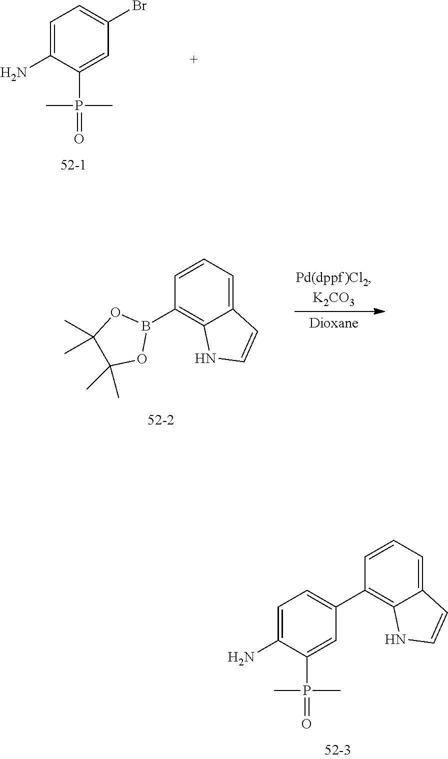 [SMILES: CP(C)(=O)c1cc(Br)ccc1N ; CC1(C)OB(c2cccc3cc[nH]c23)OC1(C)C ; CP(C)(=O)c1cc(-c2cccc3cc[nH]c23)ccc1N]

Add compounds 52-1, 52-2, Pd(dppf)Cl₂. K₂CO₃, dioxane and water to the reaction flask, heat to 110° C. under nitrogen protection, and stir with heating for 16 hr. The reaction was complete as monitored by LCMS, and then stopped. The reaction solution was poured into water (50 mL), and extracted with dichloromethane (3×50 mL); the organic phase was washed with saturated brine (3×30 mL), dried over anhydrous sodium sulfate, separated and purified by column chromatography (dichloromethane:methanol=15:1), then concentrated and dried to obtain yellow solid of desired product 52-3, MS: 285.11 [M+H]⁺

### Step 2: Synthesis of Compound 52-5

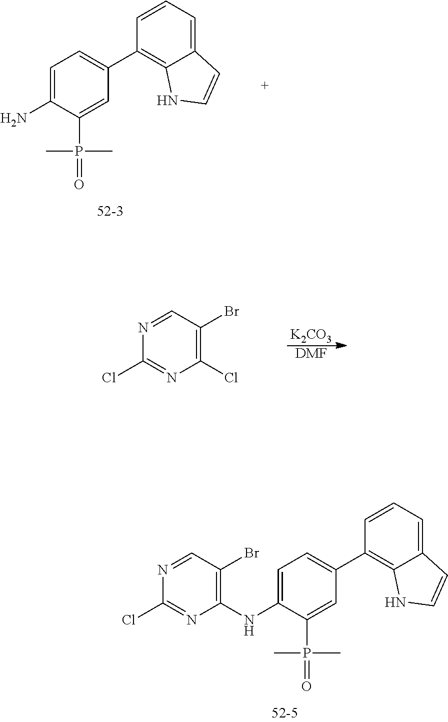

Dissolve 52-3, 5-Bromo-2,4-dichloropyrimidine to DMF in a reactor equipped with a stir bar and nitrogen protector, add K₂CO₃ at room temperature, and the gas in reactor was replaced with nitrogen for three times. After well stirring, the solution was transferred to an oil bath, heated to 100° C., and stirred at 100° C. for 16 hr. The reaction was complete as monitored by LCMS. The reaction solution was cooled down to room temperature, poured into water, and extracted with ethyl acetate (30 mL) for three times; the organic phase was concentrated by rotary evaporator. Silica gel was added to the concentrated organic phase to mix the sample, and then which was separated and purified by PE:EtOAc column with gradient from 0% to 80%. After that, it was concentrated and dried to obtain the compound 52-5, MS: 475.00 [M+H]⁺

### Step 3: Synthesis of Compound 52

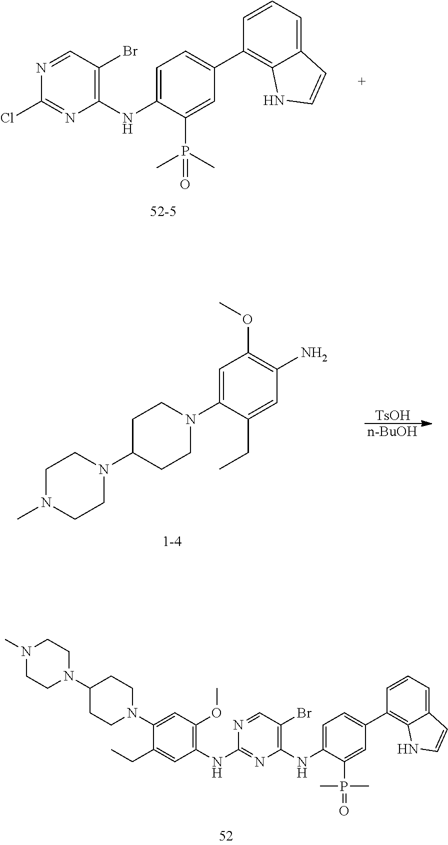

Add compounds 52-5, 1-4, p-toluenesulfonic acid and n-butanol to the reaction flask, heat to 120° C., and stir for 12 hr. The reaction was complete as monitored by LCMS, and then stopped. The reaction solution was poured into sodium carbonate solution (5 mL), and extracted with dichloromethane (3×50 mL); the organic phase was washed with saturated brine (3×30 mL), dried over anhydrous sodium sulfate, separated and purified by column chromatography (dichloromethane:methanol=12:1), and then concentrated to obtain off-white solid of compound 52, MS: 771.28 [M+H]⁺

### Example 159: Synthesis of Compound 53

### (2-((5-Chloro-2-((5-ethyl-2-methoxy-4-(4-(4-methylpiperazin-1-yl)piperidin-1-yl)phenyl)amino)pyrimidin-4-yl)amino)-5-(4-methoxypyridin-3-yl)phenyl)dimethylphosphine oxide

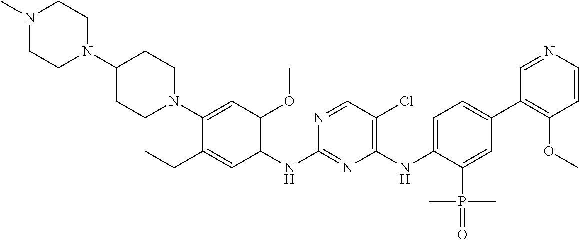

### Step 1: Synthesis of Compound 53

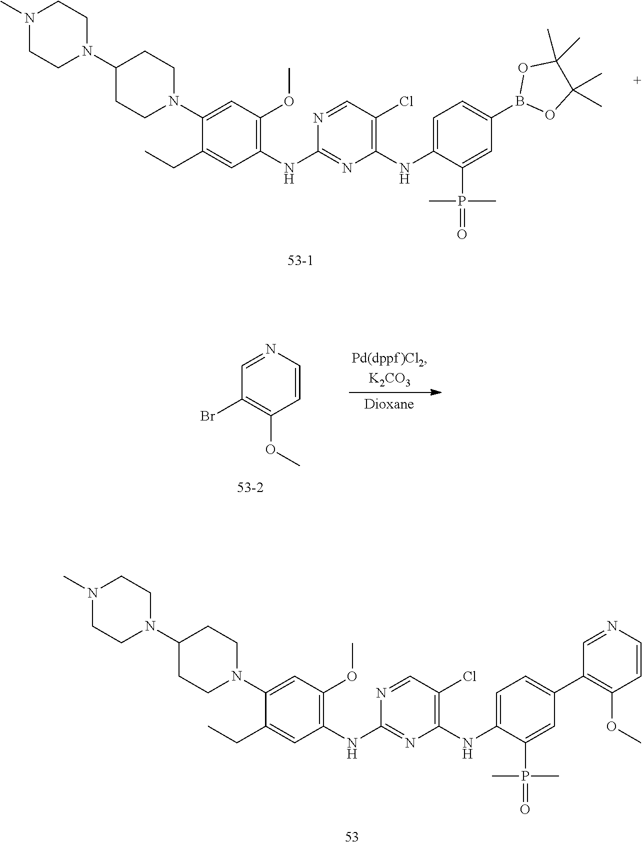

Add compounds 53-1, 53-2, Pd(dppf)Cl₂, K₂CO₃, dioxane and water to the reaction flask, heat to 110° C. under nitrogen protection and stir with heating for 16 hr. The reaction was complete as monitored by LCMS, and then stopped. The reaction solution was poured into water (50 mL), and extracted with dichloromethane (3×50 mL); the organic phase was washed with saturated brine (3×30 mL), dried over anhydrous sodium sulfate, separated and purified by column chromatography (dichloromethane:methanol=15:1), concentrated and dried to obtain the yellow solid of desired product 53, MS: 719.33 [M+H]⁺

### Example 160: Synthesis of Compound 54

### (2-((5-Bromo-2-((5-ethyl-2-methoxy-4-(4-(4-methylpiperazin-1-yl)piperidin-1-yl)phenyl)amino)amino]pyrimidin-4-yl)amino)-5-(1,4-dimethyl-1H-pyrazol-5-yl)phenyl)dimethylphosphine oxide

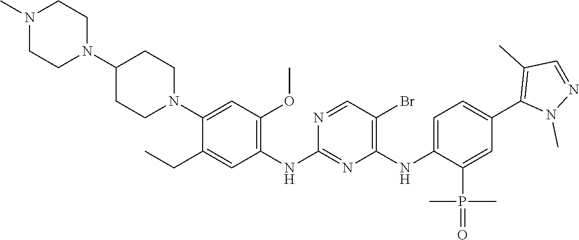 [SMILES: CCc1cc(Nc2ncc(Br)c(Nc3ccc(-c4c(C)cnn4C)cc3P(C)(C)=O)n2)c(OC)cc1N1CCC(N2CCN(C)CC2)CC1]

### Step 1: Synthesis of Compound 54-3

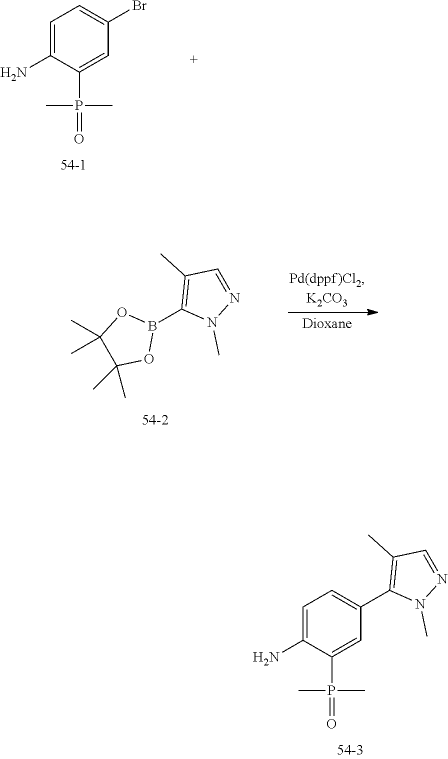 [SMILES: CP(C)(=O)c1cc(Br)ccc1N ; Cc1cnn(C)c1B1OC(C)(C)C(C)(C)O1 ; Cc1cnn(C)c1-c1ccc(N)c(P(C)(C)=O)c1]

Add compounds 54-1, 54-2, Pd(dppf)Cl₂, K₂CO₃, dioxane and water to the reaction flask, heat to 110° C. under nitrogen protection, and stir with heating for 16 hr. The reaction was complete as monitored by LCMS, and then stopped. The reaction solution was poured into water (50 mL), and extracted with dichloromethane (3×50 mL); the organic phase was washed with saturated brine (3×30 mL), dried over anhydrous sodium sulfate, separated and purified by column chromatography (dichloromethane:methanol=15:1), concentrated and dried to obtain the yellow solid of desired product 54-3, MS: 264.12 [M+H]⁺

### Step 2: Synthesis of Compound 54-5

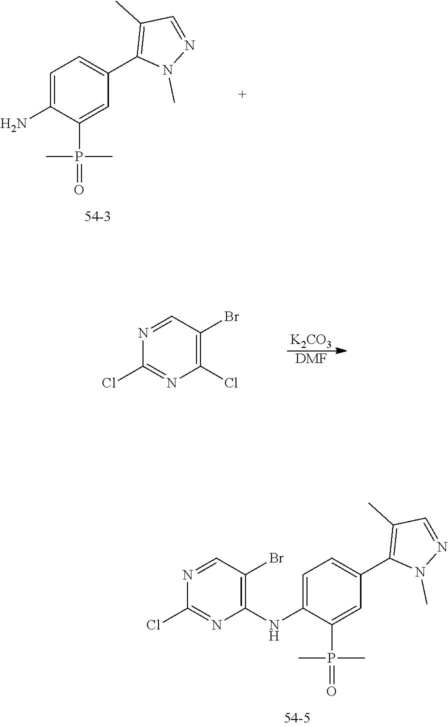

Dissolve 54-3, 5-Bromo-2,4-dichloropyrimidine to DMF in a reactor equipped with a stir bar and nitrogen protector, add K₂CO₃ at room temperature, and the gas in reactor was replaced with nitrogen for three times. After well stirring, the solution was transferred to an oil bath, heated to 100° C., and stirred at 100° C. for 16 hr. The reaction was complete as monitored by LCMS. The reaction solution was cooled down to room temperature, poured into water, and extracted with ethyl acetate (30 mL) for three times; the organic phase was concentrated by rotary evaporator. Silica gel was added to the concentrated organic phase to mix the sample, and then which was separated and purified by PE:EtOAc column with gradient from 0%-80%. After that, it was concentrated and dried to obtain the compound 54-5, MS: 454.01 [M+H]⁺

### Step 3: Synthesis of Compound 54

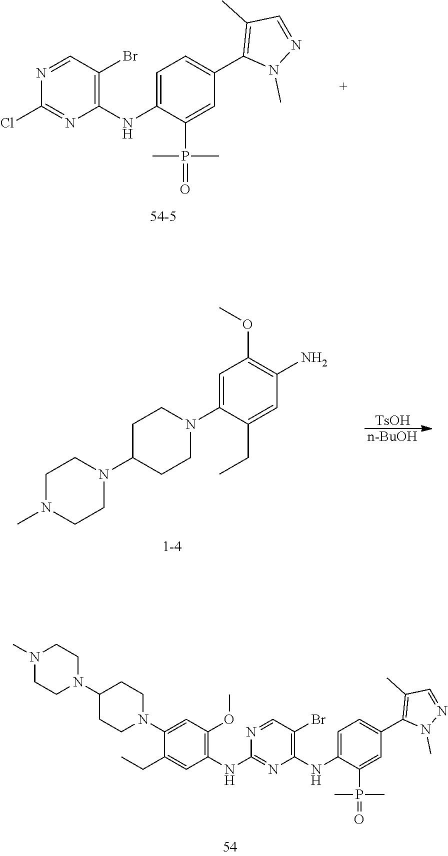

Add compounds 54-5, 1-4, p-toluenesulfonic acid and n-butanol to the reaction flask, heat to 120° C., and stir for 12 hr. The reaction was complete as monitored by LCMS, and then stopped. The reaction solution was poured into sodium carbonate solution (5 mL), and extracted with dichloromethane (3×50 mL); the organic phase was washed with saturated brine (3×30 mL), dried over anhydrous sodium sulfate, separated and purified by column chromatography (dichloromethane:methanol=12:1), and then concentrated to obtain off-white solid of compound 54, MS: 750.29 [M+H]⁺

### Example 161: Synthesis of Compound 55

### (2-((5-Bromo-2-((5-ethyl-2-methoxy-4-(4-(4-methylpiperazin-1-yl)piperidin-1-yl)phenyl)amino)amino]pyrimidin-4-yl)amino)-5-(1-methyl-1H-pyrrolo[2,3-b]pyridin-5-yl)phenyl)dimethylphosphine oxide

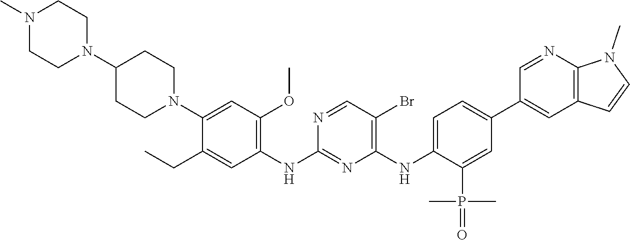 [SMILES: CCc1cc(Nc2ncc(Br)c(Nc3ccc(-c4cnc5c(ccn5C)c4)cc3P(C)(C)=O)n2)c(OC)cc1N1CCC(N2CCN(C)CC2)CC1]

### Step 1: Synthesis of Compound 55-3

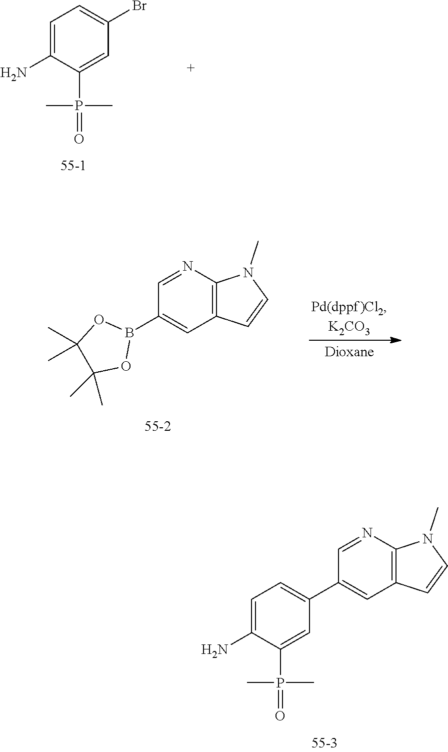 [SMILES: CP(C)(=O)c1cc(Br)ccc1N ; Cn1ccc2cc(B3OC(C)(C)C(C)(C)O3)cnc21 ; Cn1ccc2cc(-c3ccc(N)c(P(C)(C)=O)c3)cnc21]

Add compounds 55-1, 55-2, Pd(dppf)Cl₂, K₂CO₃, dioxane and water successively to the reaction flask, heat to 110° C. under nitrogen protection, and stir with heating for 16 hr. The reaction was complete as monitored by LCMS, and then stopped. The reaction solution was poured into water (50 mL), and extracted with dichloromethane (3×50 mL); the organic phase was washed with saturated brine (3×30 mL), dried over anhydrous sodium sulfate, separated and purified by column chromatography (dichloromethane:methanol=15:1), then concentrated and dried to obtain yellow solid of desired product 55-3, MS: 300.12 [M+H]⁺

### Step 2: Synthesis of Compound 55-5

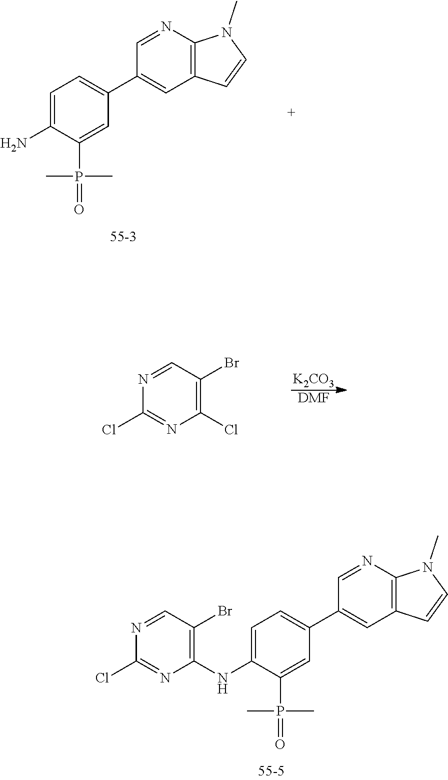

Dissolve 55-3 and 5-Bromo-2,4-dichloropyrimidine in DMF in a reactor equipped with a stir bar and nitrogen protector, add K₂CO₃ at room temperature; the gas in reactor was replaced with nitrogen for three times. After well stirring, the solution was transferred to an oil bath, heated to 100° C., and stirred at 100° C. for 16 hr. The reaction was complete as monitored by LCMS. The reaction solution was cooled down to room temperature, poured into water, and extracted with ethyl acetate (30 mL) for three times; the organic phase was concentrated by rotary evaporator. Silica gel was added to the concentrated organic phase to mix the sample, and then which was separated and purified by PE:EtOAc column with gradient from 0% to 80%. After that, it was concentrated and dried to obtain the compound 55-5, MS: 490.01 [M+H]⁺

### Step 3: Synthesis of Compound 55

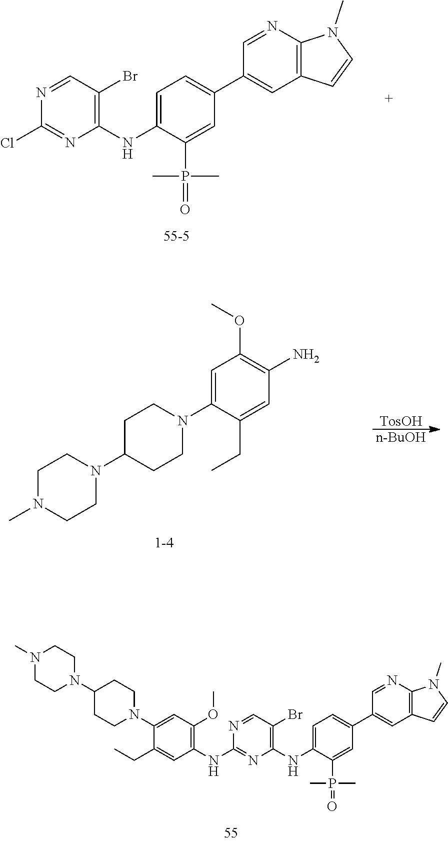

Add compounds 55-5, 1-4, p-toluenesulfonic acid and n-butanol successively into the reaction flask, heat to 120° C., and stir for 12 hr. The reaction was complete as monitored by LCMS, and then stopped. The reaction solution was poured into sodium carbonate solution (5 mL), and extracted with dichloromethane (3×50 mL); the organic phase was washed with saturated brine (3×30 mL), dried over anhydrous sodium sulfate, separated and purified by column chromatography (dichloromethane:methanol=12:1), and then concentrated to obtain an off-white solid compound 55, MS: 786.29 [M+H]⁺

### Example 162: Synthesis of Compound 56

### (2-((5-Chloro-2-((5-ethyl-2-methoxy-4-(4-(4-methylpiperazin-1-yl)piperidin-1-yl)phenyl)amino)pyrimidin-4-yl)amino)-5-(1-methyl-1H-pyrrolo[2,3-b]pyridin-4-yl)phenyl)dimethylphosphine oxide

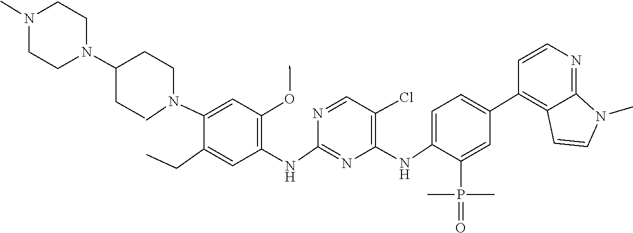

### Step 1: Synthesis of Compound 56

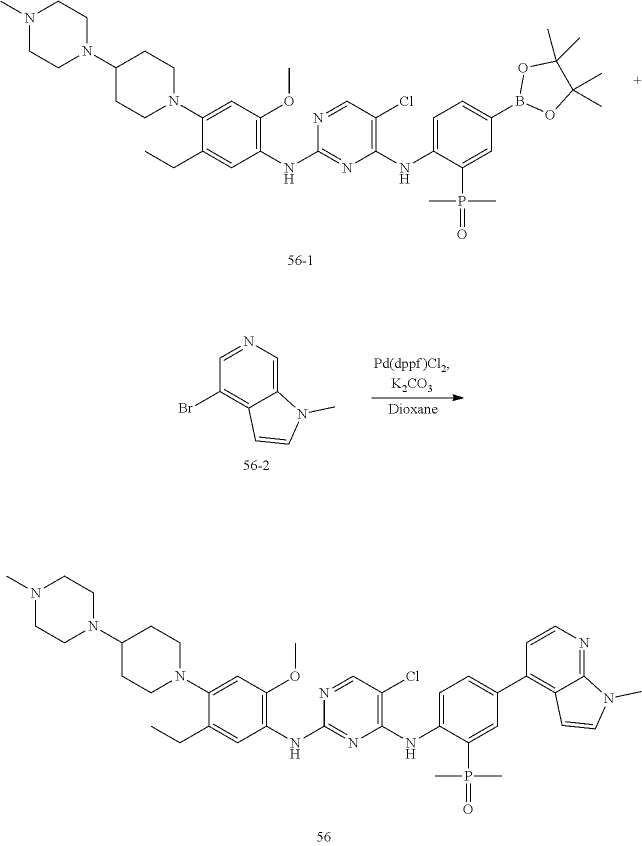

Add compounds 56-1, 56-2, Pd(dppf)Cl₂, K₂CO₃, dioxane and water successively to the reaction flask, heat to 110° C. under nitrogen protection and stir with heating for 16 hr. The reaction was complete as monitored by LCMS, and then stopped. The reaction solution was poured into water (50 mL), and extracted with dichloromethane (3×50 mL); the organic phase was washed with saturated brine (3×30 mL), dried over anhydrous sodium sulfate, separated and purified by column chromatography (dichloromethane:methanol=15:1), and then concentrated and dried to obtain the yellow solid of desired product 56, MS: 742.35 [M+H]⁺

### Example 163: Synthesis of Compound 57

### 3-(4-((5-(Chloro-2-((5-ethyl-2-methoxy-4-(4-(4-methylpiperazin-1-yl)piperidin-1-yl)phenyl)amino)pyrimidin-4-yl)amino)-3-(dimethylphosphoryl)phenyl)-1-methyl-1H-pyrazole-4-carbonitrile

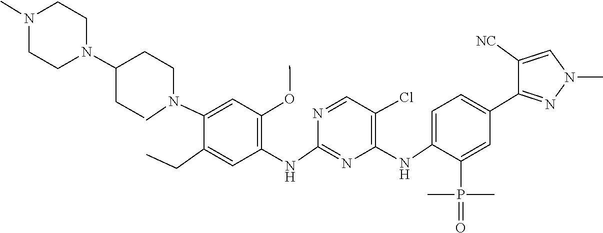

### Step 1: Synthesis of Compound 57

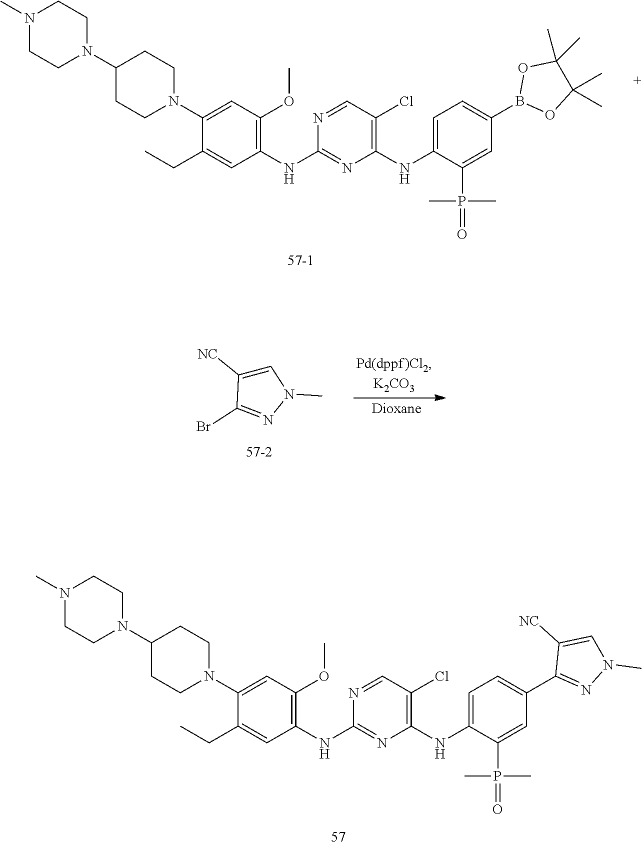

Add compounds 57-1, 57-2, Pd(dppf)Cl₂, K₂CO₃, dioxane and water successively to the reaction flask, heat to 110° C. under nitrogen protection, and stir with heating for 16 hr. The reaction was complete as monitored by LCMS, and then stopped. The reaction solution was poured into water (50 mL), and extracted with dichloromethane (3×50 ml); the organic phase was washed with saturated brine (3×30 mL), dried over anhydrous sodium sulfate, separated and purified by column chromatography (dichloromethane:methanol=15:1), concentrated and dried to obtain the yellow solid of desired product 57, MS: 717.33 [M+H]⁺

### Example 164: Synthesis of Compound 58

### (2-((5-Chloro-2-((5-ethyl-2-methoxy-4-(4-(4-methylpiperazin-1-yl)piperidin-1-yl)phenyl)amino)pyrimidin-4-yl)amino)-5-(3-ethoxy-5-methylisoxazol-4-yl)phenyl)dimethylphosphine oxide

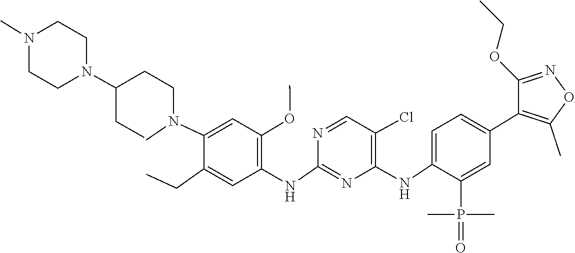 [SMILES: CCOc1noc(C)c1-c1ccc(Nc2nc(Nc3cc(CC)c(N(C)CCC(CC)N4CCN(C)CC4)cc3OC)ncc2Cl)c(P(C)(C)=O)c1]

### Step 1: Synthesis of Compound 58

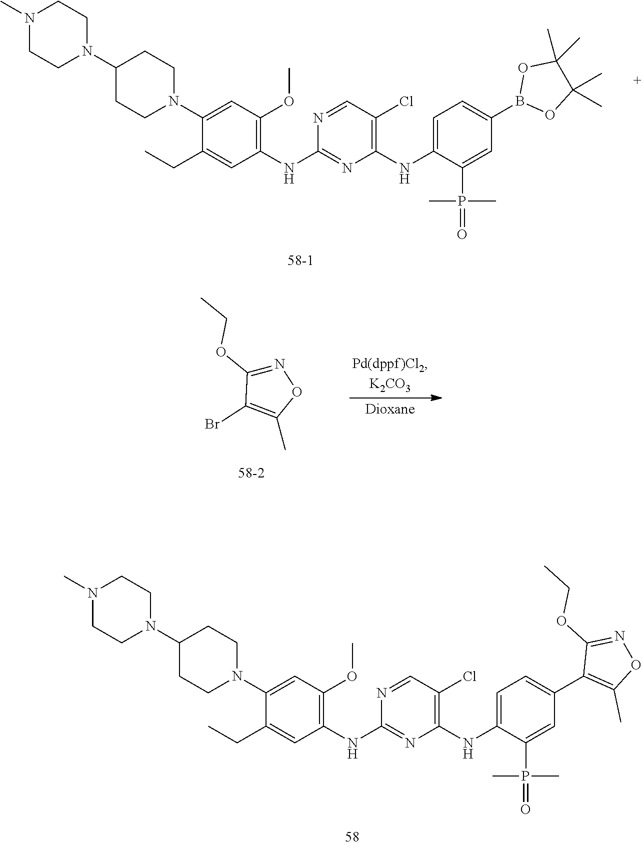

Add compounds 58-1, 58-2, Pd(dppf)Cl₂, K₂CO₃, dioxane and water successively to the reaction flask, heat to 110° C. under nitrogen protection, and stir with heating for 16 hr. The reaction was complete as monitored by LCMS, and then stopped. The reaction solution was poured into water (50 mL), and extracted with dichloromethane (3×50 mL); the organic phase was washed with saturated brine (3×30 mL), dried over anhydrous sodium sulfate, separated and purified by column chromatography (dichloromethane:methanol=15:1), concentrated and dried to obtain the yellow solid of desired product 58, MS: 737.34 [M+H]⁺

### Example 165: Synthesis of Compound 59

### (2-((5-Bromo-2-((5-ethyl-2-methoxy-4-(4-(4-methylpiperazin-1-yl)piperidin-1-yl)phenyl)amino)pyrimidin-4-yl)amino)-5-(6-methoxypyrazin-2-yl)phenyl)dimethylphosphine oxide

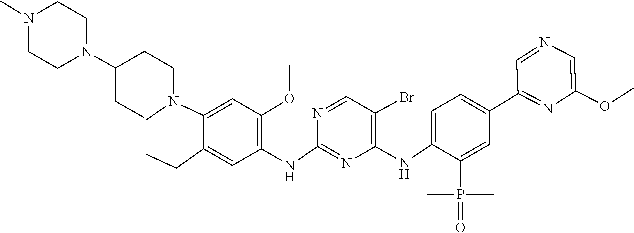

### Step 1: Synthesis of Compound 59-2

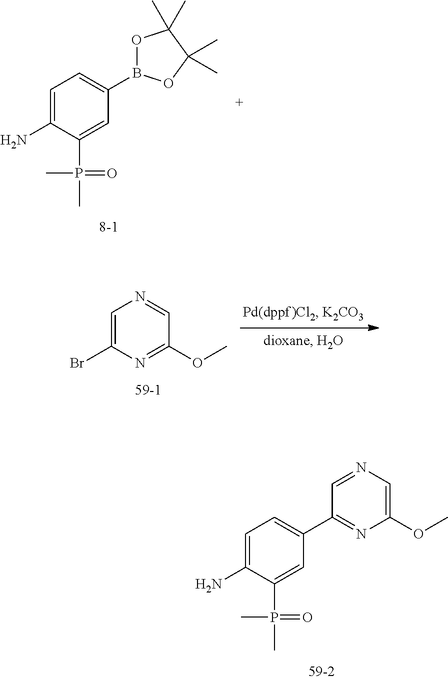

The compound 39-2 was synthesized as the method described or compound 8-3 using compound 59-1 instead of raw compound 8-2. For compound 59-2, MS: 278.10 [M+H]⁺

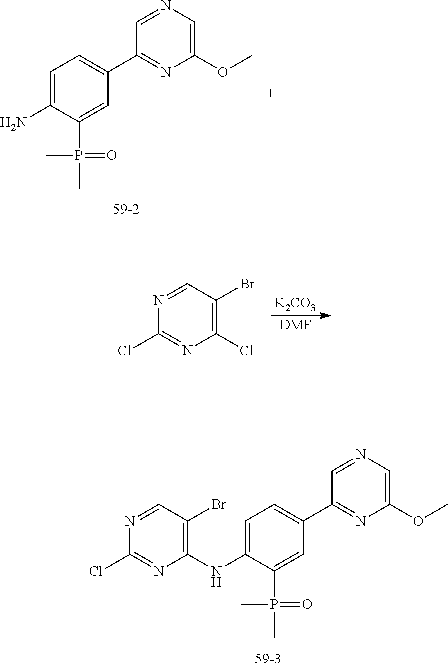

### Step 2: Synthesis of Compound 59-3

The compound 59-3 was synthesized as the method described for compound 1-10 using compound 59-2 instead of raw compound 1-8. For compound 59-3, MS: 467.99 [M+H]⁺

### Step 3: Synthesis of Compound 59

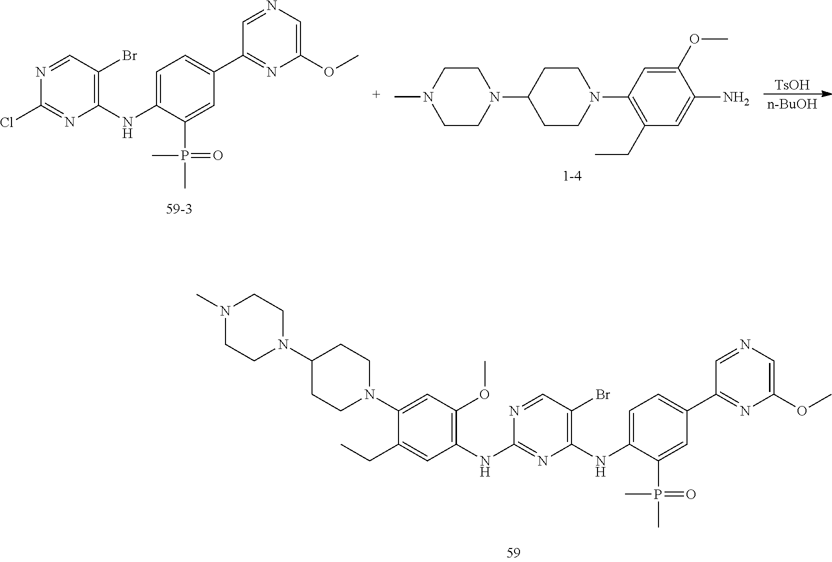

The compound 59 was synthesized as the method described for compound 1 using compound 59-3, instead of raw compound 1-10. For compound 59, MS: 764.27 [M+H]⁺

### Example 166: Synthesis of Compound 60

### (2-((5-Bromo-2-((5-ethyl-2-methoxy-4-(4-(4-methylpiperazin-1-yl)piperidin-1-yl)phenyl)amino)amino]pyrimidin-4-yl)amino)-5-(5-methylthiazol-4-yl)phenyl)dimethylphosphine oxide

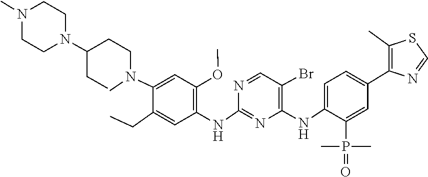 [SMILES: CCc1cc(Nc2ncc(Br)c(Nc3ccc(-c4ncsc4C)cc3P(C)(C)=O)n2)c(OC)cc1N(C)CCC(CC)N1CCN(C)CC1]

### Step 1: Synthesis of Compound 60-2

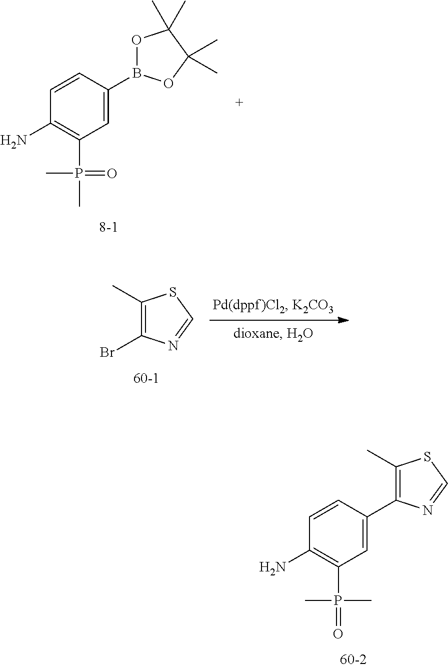

The compound 60-2 was synthesized as the method described for compound 8-3 using compound 60-1 instead of raw compound 8-2. For compound 60-2, MS: 267.06 [M+H]⁺

### Step 2: Synthesis of Compound 60-3

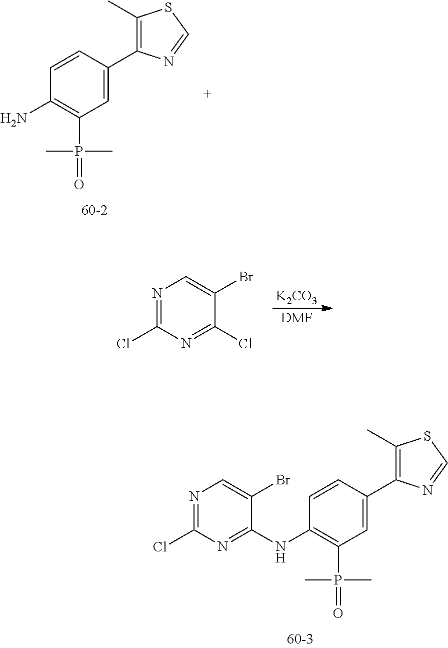

The compound 60-3 was synthesized as the method described for compound 1-10 using compound 60-2 instead of raw compound 1-8. For compound 60-3, MS: 456.96 [M+H]⁺

### Step 2: Synthesis of Compound 60

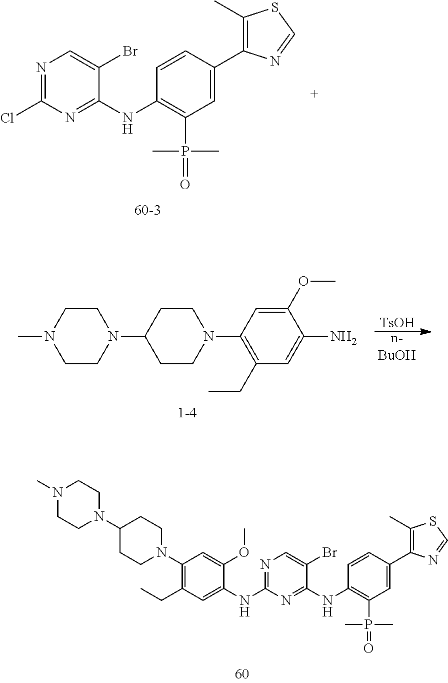

The compound 60 was synthesized as the method described for compound 1 using compound 60-3 instead of raw compound 1-10. For compound 60, MS: 753.24 [M+H]⁺

### Example 168: Synthesis of Compound 62

### (2-((5-Bromo-2-((5-ethyl-2-methoxy-4-(4-(4-methylpiperazin-1-yl)piperidin-1-yl)phenyl)amino]pyrimidin-4-yl)amino)-5-(1-methyl-1H-1,2,4-triazol-5-yl)phenyl)dimethylphosphine oxide

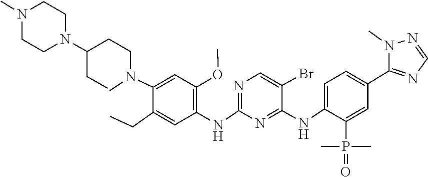 [SMILES: CCc1cc(Nc2ncc(Br)c(Nc3ccc(-c4ncnn4C)cc3P(C)(C)=O)n2)c(OC)cc1N(C)CCC(CC)N1CCN(C)CC1]

### Step 1: Synthesis of Compound 62-2

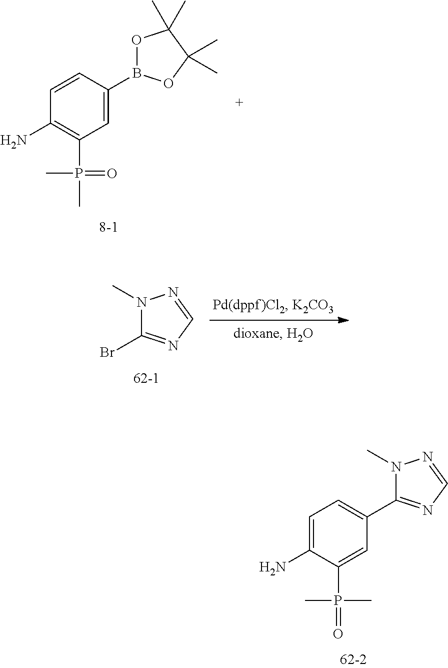

The compound 62-2 was synthesized as the method described for compound 8-3 using compound 62-1 instead of raw compound 8-2. For compound 62-2, MS: 251.10 [M+H]⁺

### Step 3: Synthesis of Compound 62-3

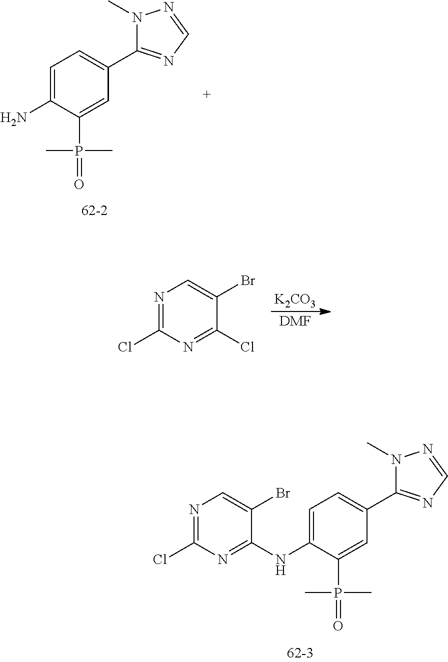

The compound 62-3 was synthesized as the method described for compound 1-10 using compound 62-2 instead of raw compound 1-8. For compound 62-3, MS: 440.99 [M+H]⁺

### Step 3: Synthesis of Compound 62

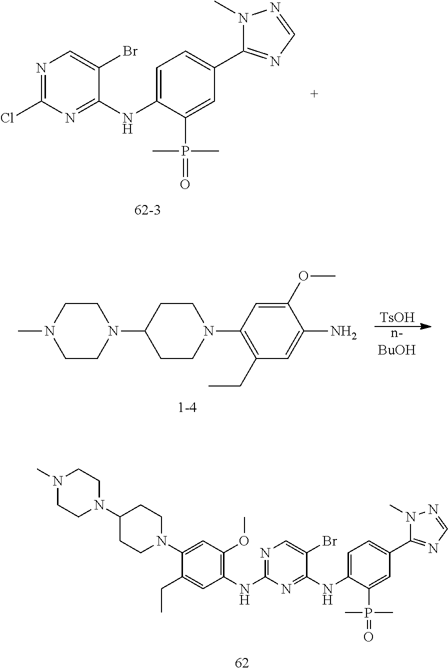

The compound 62 was synthesized as the method described for compound 1 using compound 62-3 instead of raw compound 1-10. For compound 62, MS: 737.27 [M+H]⁺

### Example 169: Synthesis of Compound 63

### (2-((5-Bromo-2-((5-ethyl-2-methoxy-4-(4-(4-methylpiperazin-1-yl)piperidin-1-yl)phenyl)amino)amino]pyrimidin-4-yl)amino)-5-(3-methoxy-1-methyl-1H-pyrazol-4-yl)phenyl)dimethylphosphine oxide

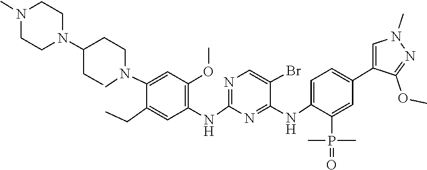

### Step 1: Synthesis of Compound 63-2

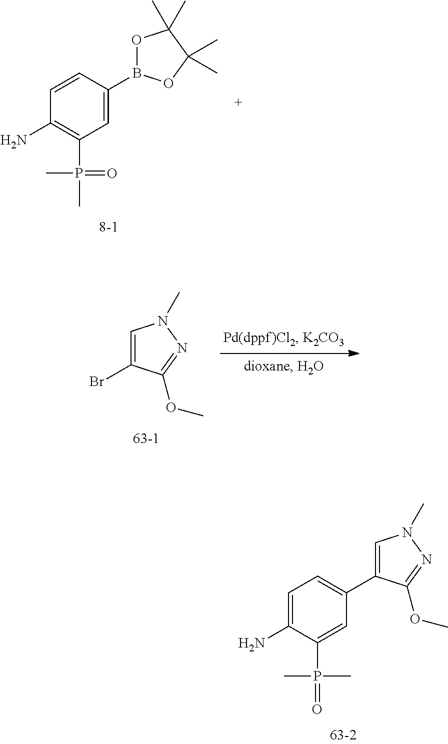

The compound 63-2 was synthesized as the method described for compound 8-3 using compound 63-1 instead of raw compound 8-2. For compound 63-2, MS: 280.11 [M+H]⁺

### Step 2: Synthesis of Compound 61-2

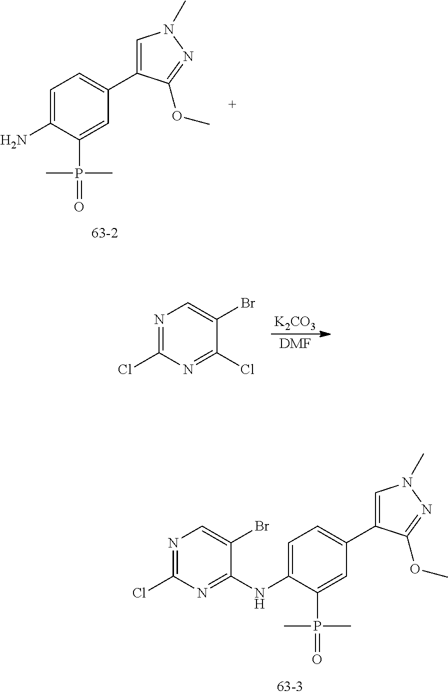

The compound 63-3 was synthesized as the method described for compound 1-10 using compound 63-2 instead of raw compound 1-8. For compound 63-, MS: 470.01 [M+H]⁺

### Step 3: Synthesis of Compound 63

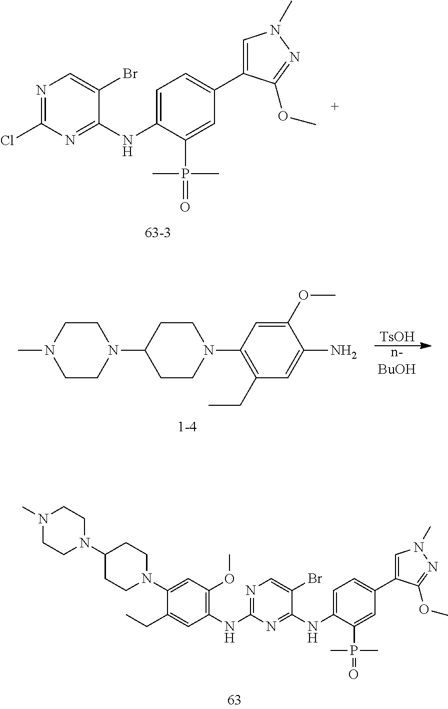

The compound 63 was synthesized as the method described for compound 1 using compound 63-3 instead of raw compound 1-10. For compound 63, MS: 766.29 [M+H]⁺

### Example 170: Synthesis of Compound 64

### 4-(4-((5-Bromo-2-((5-ethyl-2-methoxy-4-(4-(4-methylpiperazin-1-yl)piperidin-1-yl)phenyl)amino]pyrimidin-4-yl)amino)-3-(dimethylphosphoryl)phenyl)-1-methyl-1H-pyrazole-3-carbonitrile

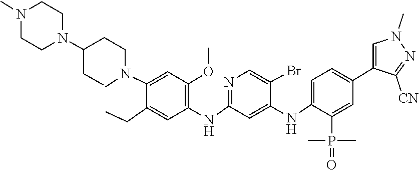 [SMILES: CCc1cc(Nc2cc(Nc3ccc(-c4cn(C)nc4C#N)cc3P(C)(C)=O)c(Br)cn2)c(OC)cc1N(C)CCC(CC)N1CCN(C)CC1]

### Step 1: Synthesis of Compound 64-2

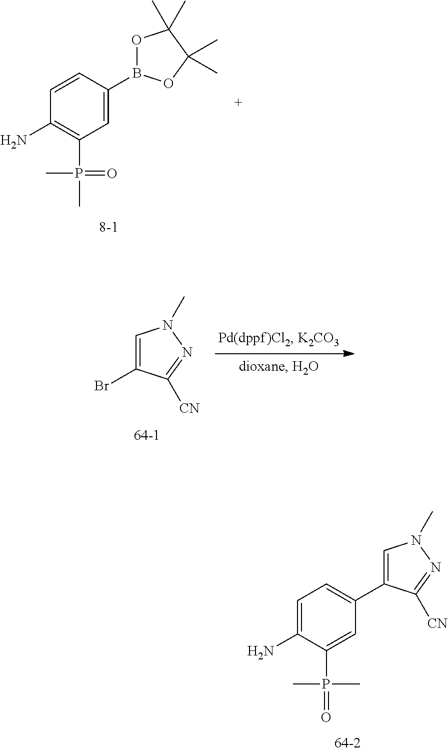

The compound 64-2 was synthesized as the method described for compound 8-3 using compound 64-1 instead of raw compound 8-2. For compound 64-2, MS: 275.10 [M+H]⁺

### Step 2: Synthesis of Compound 64-3

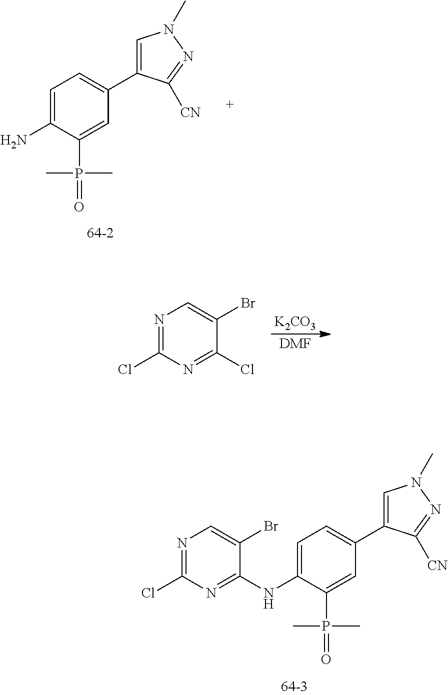

The compound 64-3 was synthesized as the method described for compound 1-10 using compound 64-2 instead of raw compound 1-8. For compound 64-3, MS: 464.99 [M+H]⁺

### Step 3: Synthesis of Compound 64

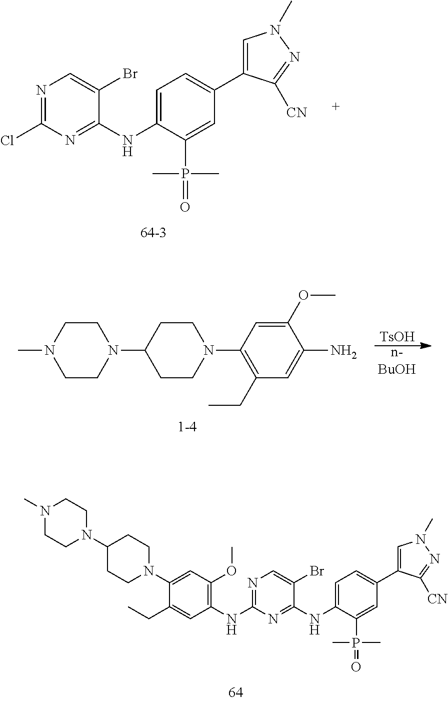

The compound 64 was synthesized as the method described for compound 1 using compound 64-3 instead of raw compound 1-10. For compound 64, MS: 761.27 [M+H]⁺

### Example 171: Synthesis of Compound 65

### (2-((5-Chloro-2-((5-ethyl-2-methoxy-4-(4-(4-methylpiperazin-1-yl)piperidin-1-yl)phenyl)amino)pyrimidin-4-yl)amino)-5-(4-methoxypyrimidin-5-yl)phenyl)dimethylphosphine oxide

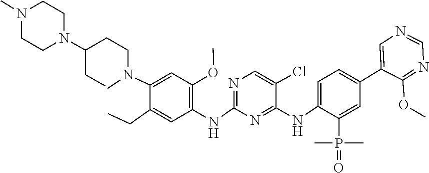

### Step 1: Synthesis of Compound 65

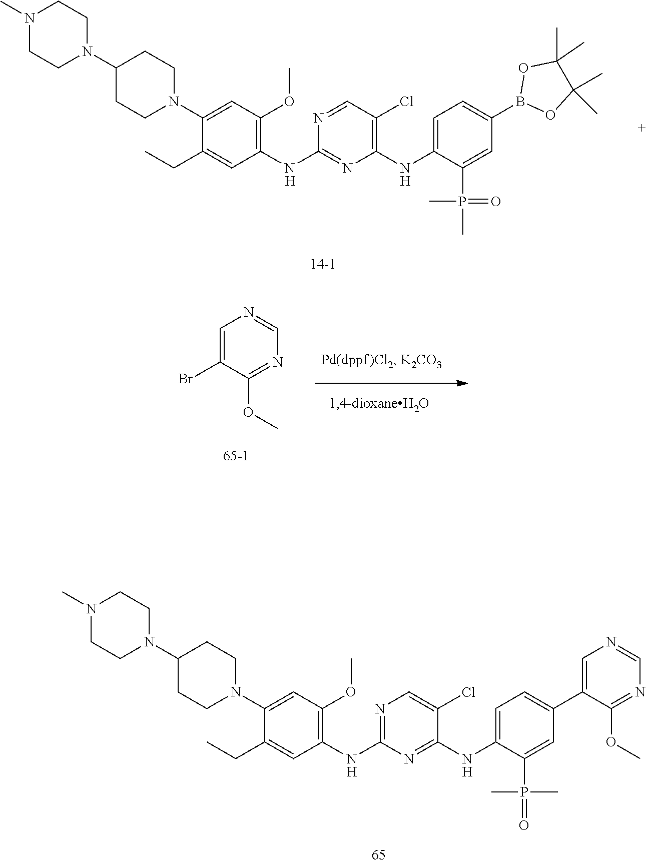

The compound 65 was synthesized as the method described for compound 14 using compound 65-1 instead of raw compound 14-2. For compound 65, MS: 720.32 [M+H]⁺

### Example 172: Synthesis of Compound 66

### (2-((5-Chloro-2-((5-ethyl-2-methoxy-4-(4-(4-methylpiperazin-1-yl)piperidin-1-yl)phenyl)amino)pyrimidin-4-yl)amino)-5-(4-methylpyrimidin-5-yl)phenyl)dimethylphosphine oxide

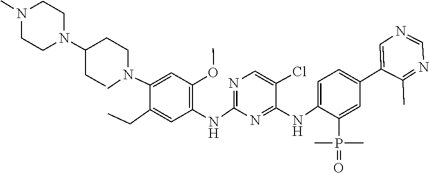 [SMILES: CCc1cc(Nc2ncc(Cl)c(Nc3ccc(-c4cncnc4C)cc3P(C)(C)=O)n2)c(OC)cc1N(C)CCC(CC)N1CCN(C)CC1]

### Step 1: Synthesis of Compound 66

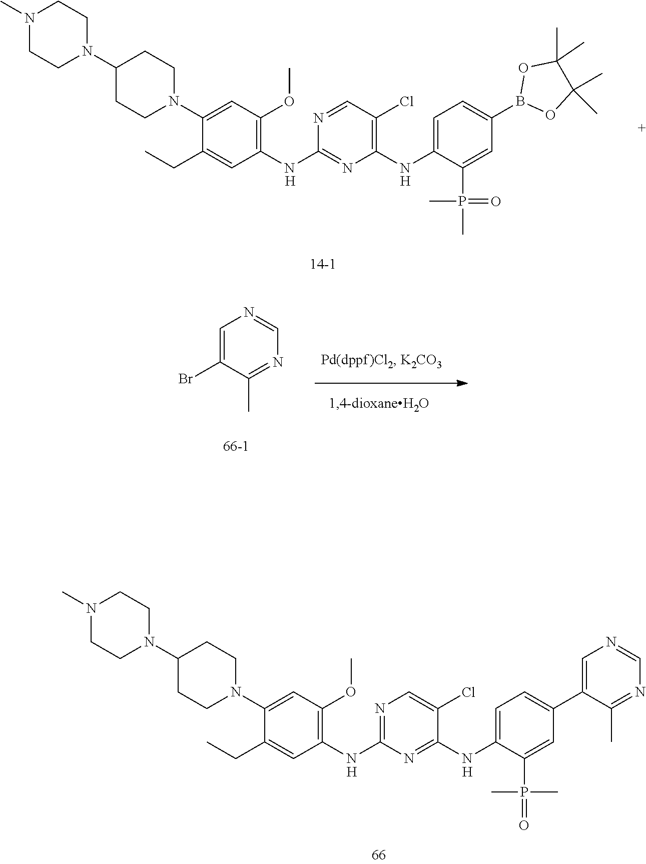

The compound 66 was synthesized as the method described for compound 14 using compound 66-1 instead of raw compound 14-2. For compound 66, MS: 704.33 [M+H]⁺

### Example 173: Synthesis of Compound 67

### (2-((5-Chloro-2-((5-ethyl-2-methoxy-4-(4-(4-methylpiperazin-1-yl)piperidin-1-yl)phenyl)amino)pyrimidin-4-yl)amino)-5-(2-methylpyrimidin-4-yl)phenyl)dimethylphosphine oxide

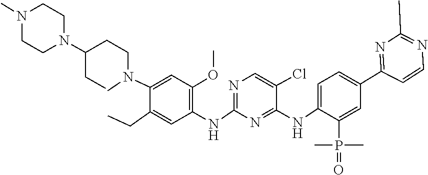 [SMILES: CCc1cc(Nc2ncc(Cl)c(Nc3ccc(-c4ccnc(C)n4)cc3P(C)(C)=O)n2)c(OC)cc1N(C)CCC(CC)N1CCN(C)CC1]

### Step 1: Synthesis of Compound 67

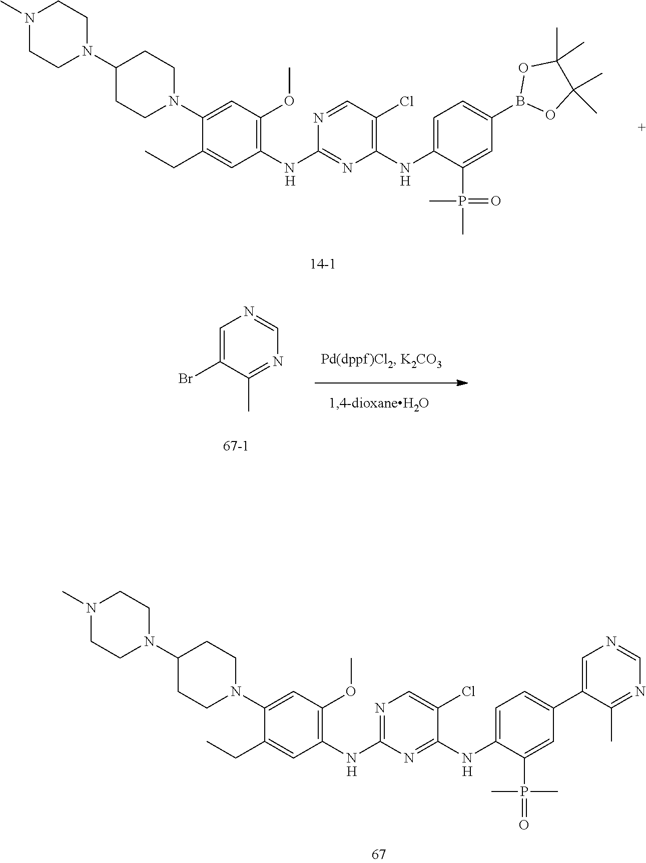

The compound 67 was synthesized as the method described for compound 14 using compound 67-1 instead of raw compound 14-2. For compound 67, MS: 704.33 [M+H]⁺

### Example 174: Synthesis of Compound 68

### (2-((5-Bromo-2-((5-ethyl-2-methoxy-4-(4-(4-methylpiperazin-1-yl)piperidin-1-yl)phenyl)amino)pyrimidin-4-yl)amino)-5-(1-methyl-1H-imidazol-4-yl)phenyl)dimethylphosphine oxide

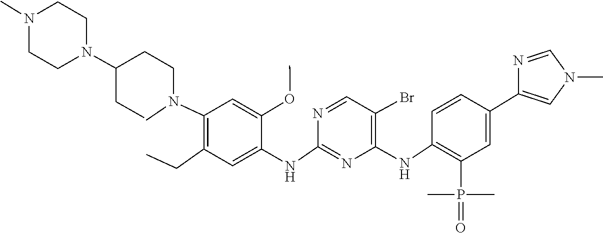

### Step 1: Synthesis of Compound 68-3

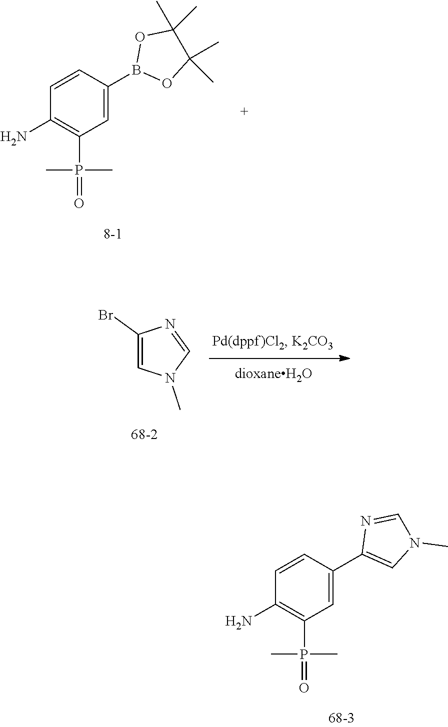

Add compounds 8-1 (200 mg), 68-2 (163 mg), K₂CO₃ (187 mg), Pd(dppf)Cl₂ (100 mg), dioxane (10 ml) and water (2 ml) successively to the reaction flask, heat to 100° C. under nitrogen protection, and stir with heating for 12 hr. The reaction was complete as monitored by LCMS, and then stopped. After adding water (20 mL), the reaction solution was extracted with dichloromethane (3×20 mL); the organic phase was washed with saturated brine (3×30 mL), dried over anhydrous sodium sulfate, separated and purified by PTLC (dichloromethane:methanol=13:1). The solvent was removed to obtain the brown solid of desired product (100 mg), MS: 250.10 [M+H]⁺

### Step 2: Synthesis of Compound 684

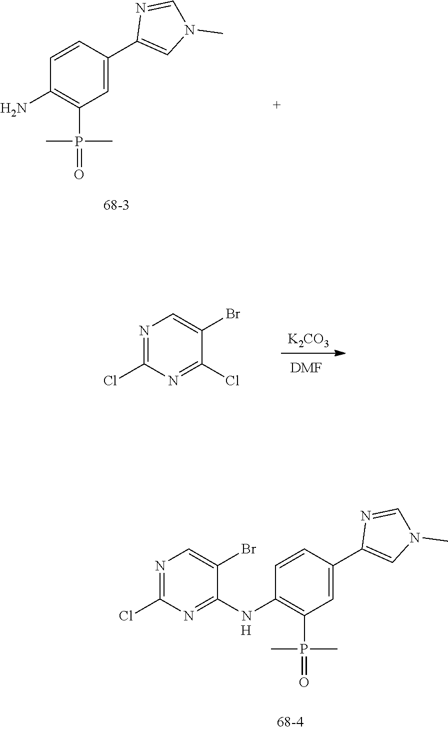

The compound 68-4 was synthesized as the method described for compound 1-10 using compound 68-3 instead of raw compound 1-8. For compound 68-4, MS: 440.00 [M+H]⁺

### Step 3: Synthesis of Compound 68

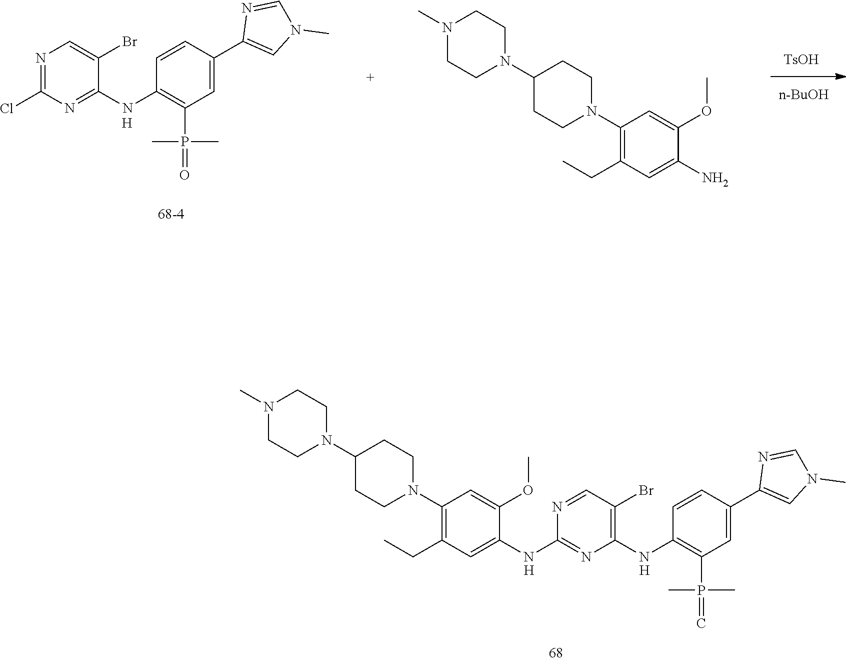

The compound 68 was synthesized as the method described for compound 1 using compound 68-4 instead of raw compound 1-10. For compound 68, MS: 736.28 [M+H]⁺

### Example 175: Synthesis of Compound 69

### (2-((5-Bromo-2-((5-ethyl-2-methoxy-4-(4-(4-methylpiperazin-1-yl)piperidin-1-yl)phenyl))amino)pyrimidin-4-yl)amino)-5-(1-cyclopropyl-1H-imidazol-2-yl)phenyl)dimethylphosphine oxide

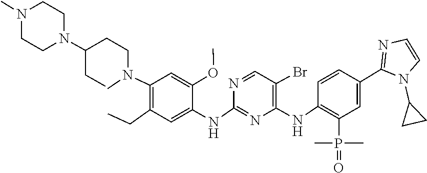 [SMILES: CCc1cc(Nc2ncc(Br)c(Nc3ccc(-c4nccn4C4CC4)cc3P(C)(C)=O)n2)c(OC)cc1N(C)CCC(CC)N1CCN(C)CC1]

### Step 1: Synthesis of Compound 69-2

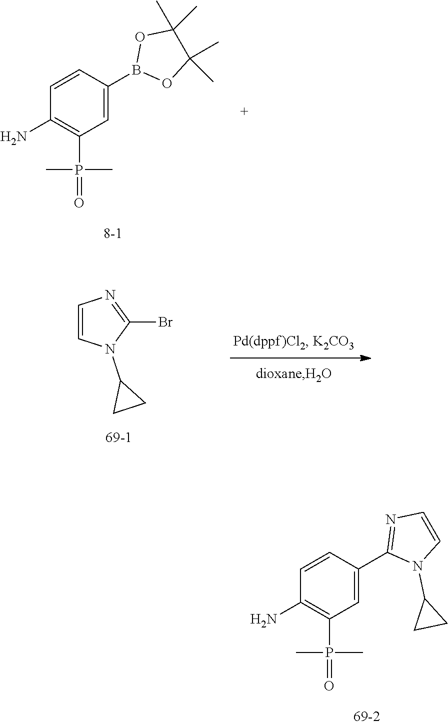 [SMILES: CC1(C)OB(c2ccc(N)c(P(C)(C)=O)c2)OC1(C)C ; Brc1nccn1C1CC1 ; CP(C)(=O)c1cc(-c2nccn2C2CC2)ccc1N]

The compound 69-2 was synthesized as the method described for compound 5-3 using compound 69-1 instead of raw compound 5-2. For compound 69-2, MS: 276.12 [M+H]⁺

### Step 2: Synthesis of Compound 69-3

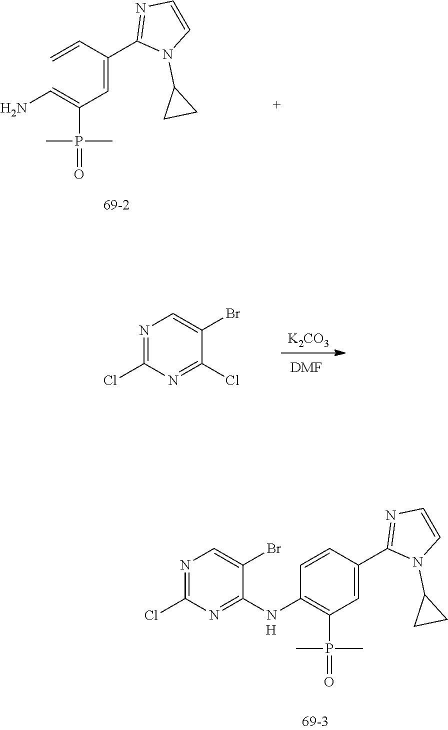

The compound 69-3 was synthesized as the method described for compound 1-10 using compound 69-2 instead of raw compound 1-8. For compound 69-3, MS: 466.01 [M+H]⁺

### Step 2: Synthesis of Compound 69

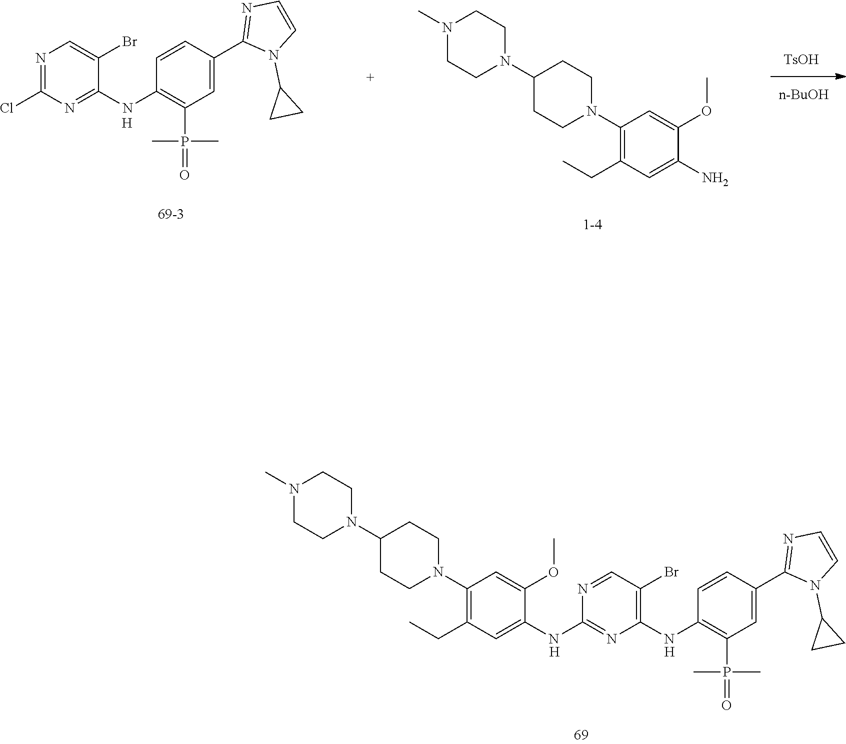

The compound 69 was synthesized as the method described for compound 1 using compound 69-3 instead of raw compound 1-10. For compound 69, MS: 762.29 [M+H]⁺

### Example 176: Synthesis of Compound 70

### (2-((5-Bromo-2-((5-ethyl-2-methoxy-4-(4-(4-methylpiperazin-1-yl)piperidin-1-yl)phenyl)(amino)pyrimidin-4-yl)imino)-5-(5-ethylpyrimidin-2-yl)phenyl)dimethylphosphine oxide

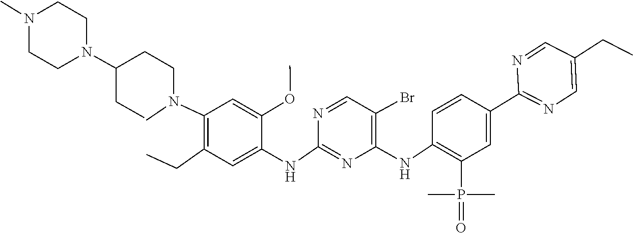 [SMILES: CCc1cnc(-c2ccc(Nc3nc(Nc4cc(CC)c(N(C)CCC(CC)N5CCN(C)CC5)cc4OC)ncc3Br)c(P(C)(C)=O)c2)nc1]

### Step 1: Synthesis of Compound 70-2

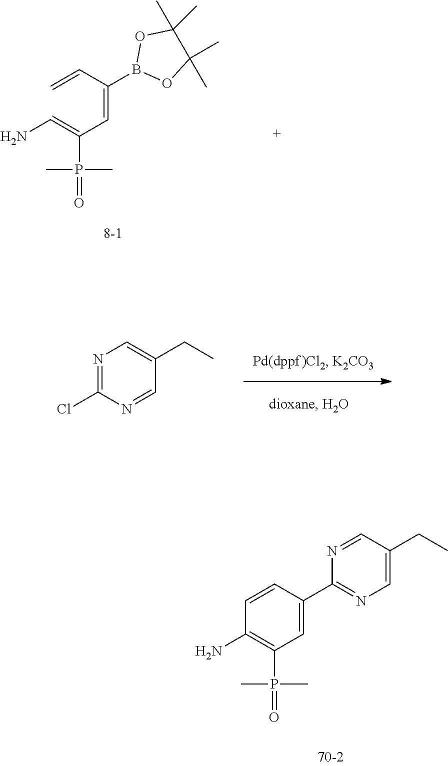

The compound 70-2 was synthesized as the method described for compound 8-3 using compound 70-1 instead of raw compound 8-2. For compound 70-2, MS: 276.12 [M+H]⁺

### Step 2: Synthesis of Compound 70-3

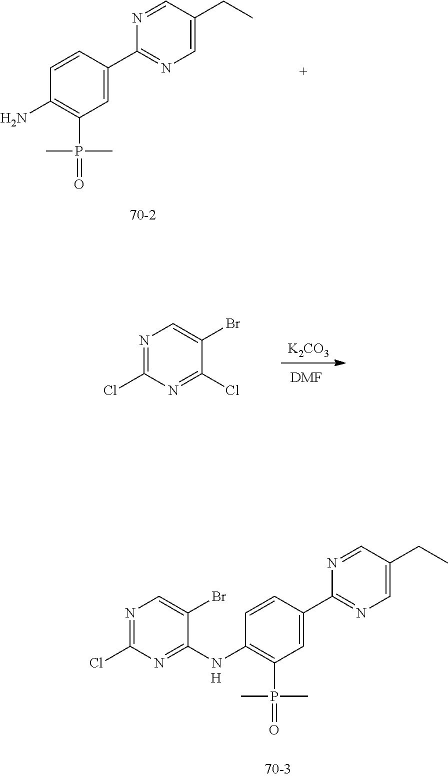

The compound 70-3 was synthesized as the method described for compound 1-10 using compound 70-2 instead of raw compound 1-8. For compound 70-3, MS: 466.01 [M+H]⁺

### Step 3: Synthesis of Compound 70

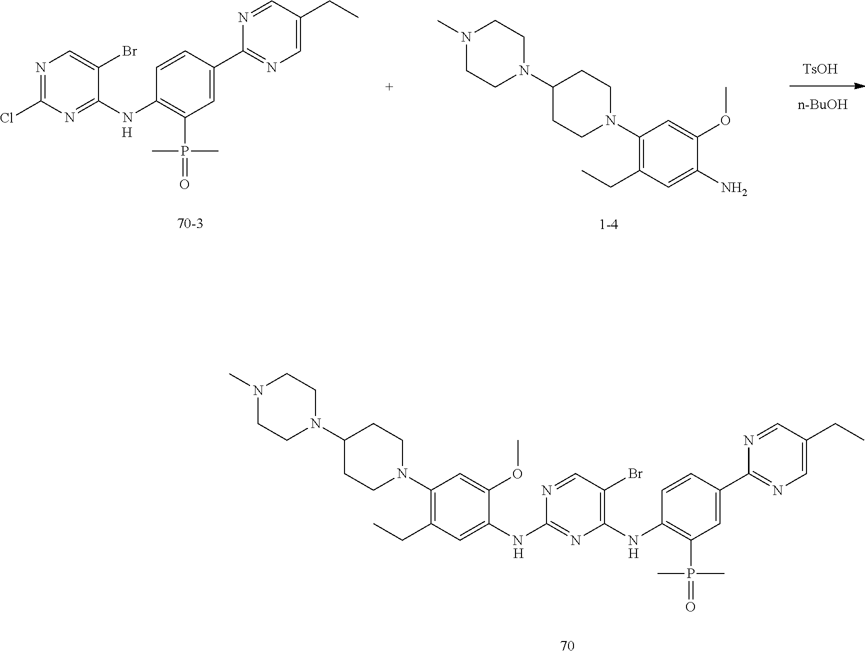

The compound 70 was synthesized as the method described for compound 1 using compound 70-3 instead of raw compound 1-10. For compound 70, MS: 762.29 [M+H]⁺

### Example 177: Synthesis of Compound 71

### (2-((5-Bromo-2-((5-ethyl-2-methoxy-4-(4-(4-methylpiperazin-1-yl)piperidin-1-yl)phenyl)amino)amino]pyrimidin-4-yl)amino)-5-(6-(dimethylamino)pyridin-2-yl)phenyl)dimethylphosphine oxide

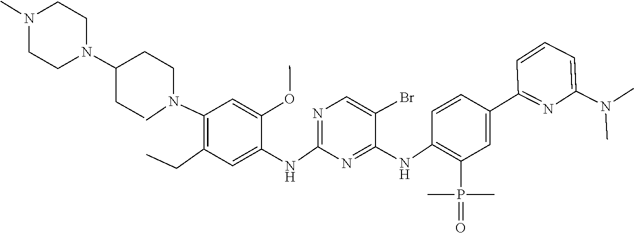

### Step 1: Synthesis of Compound 71-2

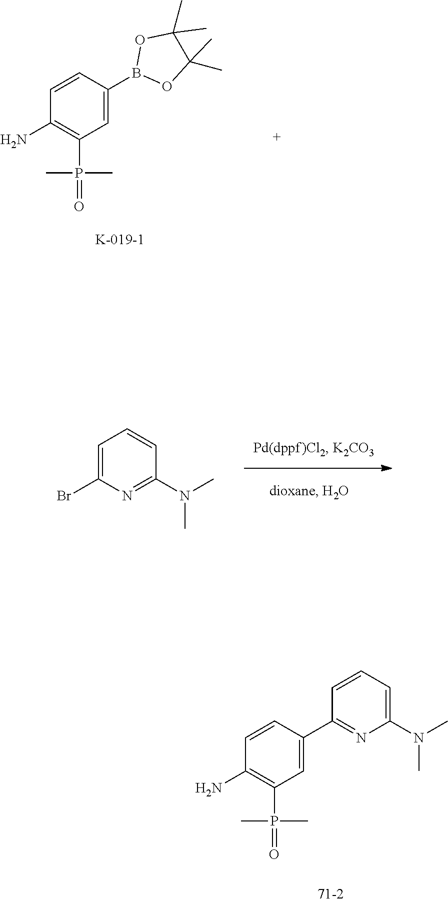

The compound 71-2 was synthesized as the method described for compound 5-3 using compound 71-1 instead of raw compound 5-2. For compound 71-2, MS: 290.13 [M+H]⁺

### Step 2: Synthesis of Compound 71-3

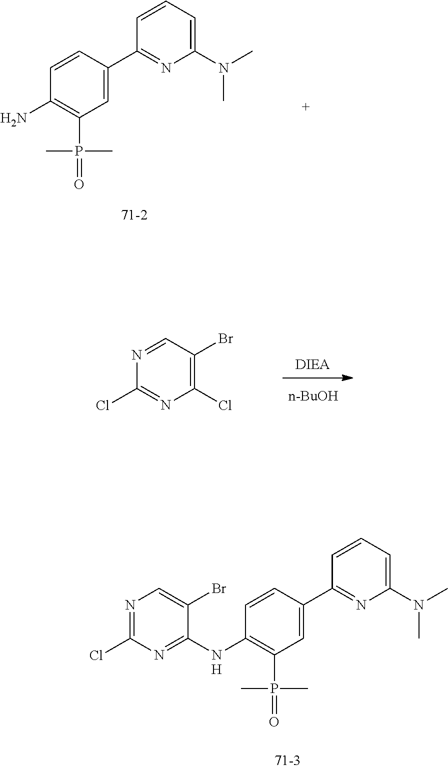

The compound 71-3 was synthesized as the method described for compound 1-10 using compound 71-2 instead of raw compound 1-8. For compound 71-3, MS: 480.03 [M+H]⁺

### Step 3: Synthesis of Compound 71

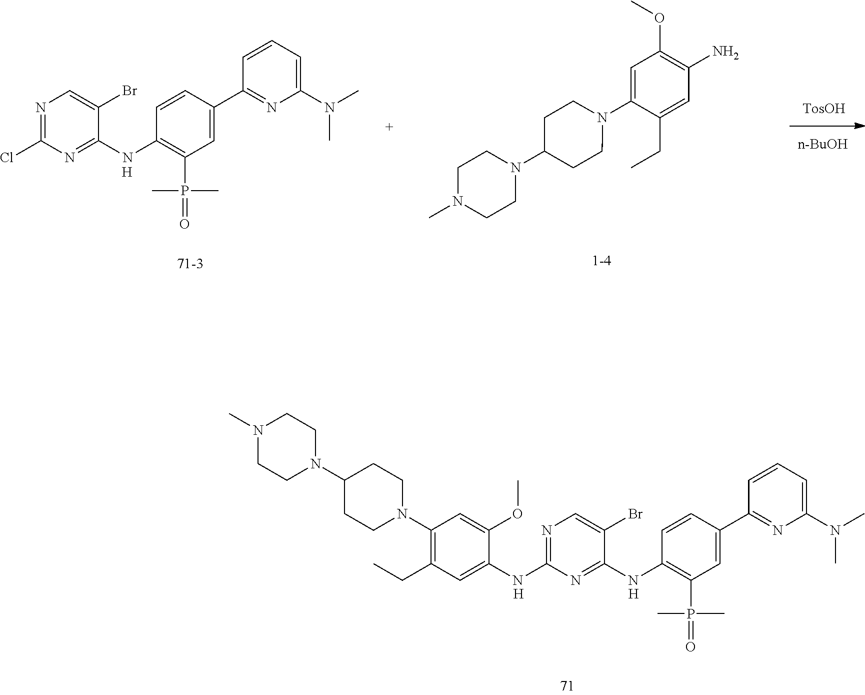

The compound 71 was synthesized as the method described for compound 1 using compound 71-3 instead of raw compound 1-10. For compound 71, MS: 776.31 [M+H]⁺

### Example 178: Synthesis of Compound 72

### (2-((5-Bromo-2-((5-ethyl-2-methoxy-4-(4-(4-methylpiperazin-1-yl)piperidin-1-yl)phenyl)(amino)amino]pyrimidin-4-yl)amino)-5-(3-methoxypyrazin-2-yl)phenyl)dimethylphosphine oxide

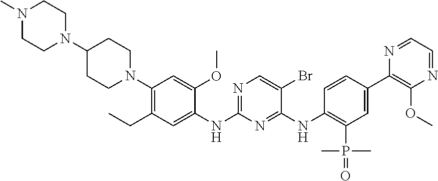

### Step 1: Synthesis of Compound 72-2

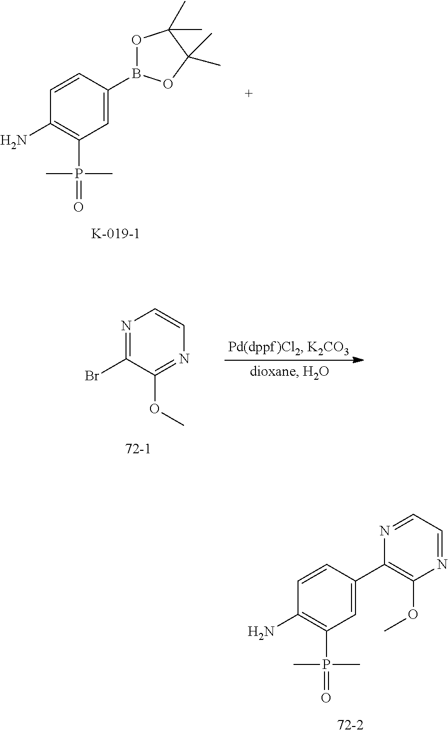

The compound 72-2 was synthesized as the method described for compound K-019-2 using compound 72-1 instead of raw compound 2-Bromothiazole. For compound 72-2, MS: 278.10 [M+H]⁺

### Step 2: Synthesis of Compound 72-3

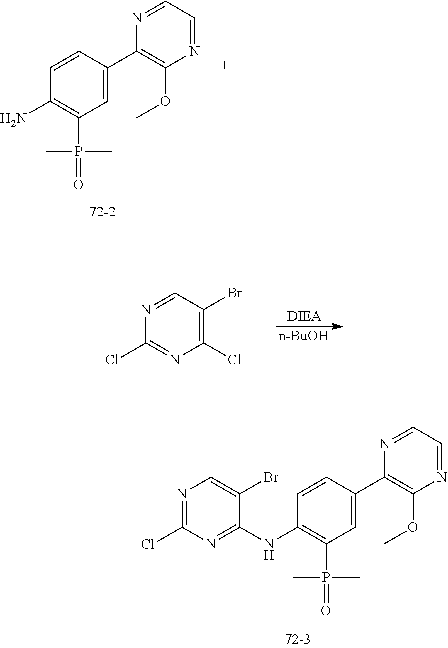

The compound 72-3 was synthesized as the method described for compound 1-10 using compound 72-2 instead of raw compound 1-8. For compound 72-3, MS: 467.99 [M+H]⁺

### Step 3: Synthesis of Compound 72

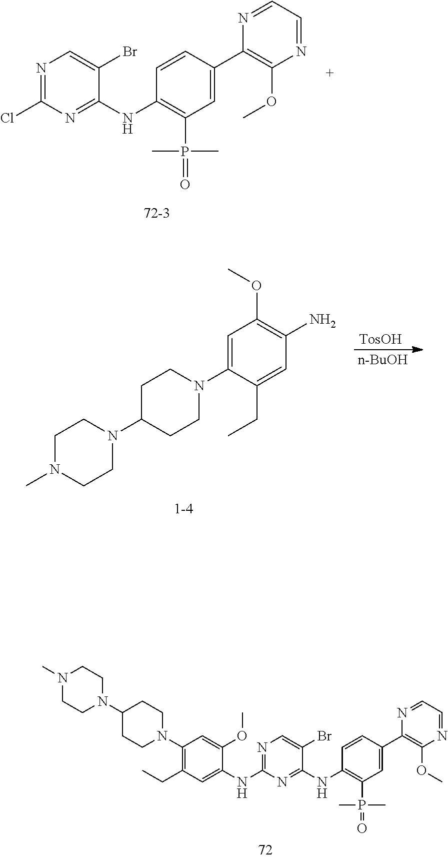

The compound 72 was synthesized as the method described for compound 1 using compound 72-3 instead of raw compound 1-10. For compound 72, MS: 764.27 [M+H]⁺

### Example 179: Synthesis of Compound 73

### (2-((5-Bromo-2-((5-ethyl-2-methoxy-4-(4-(4-methylpiperazin-1-yl)piperidin-1-yl)phenyl)amino)pyrimidin-4-yl)amino)-5-(5-isopropylpyrimidin-2-yl)phenyl)dimethylphosphine oxide

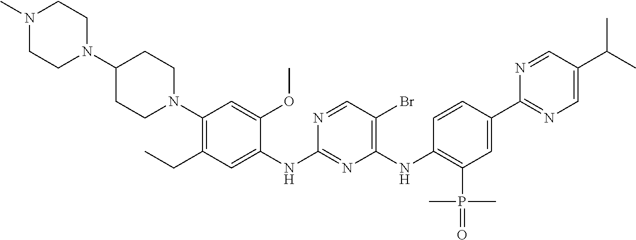 [SMILES: CCc1cc(Nc2ncc(Br)c(Nc3ccc(-c4ncc(C(C)C)cn4)cc3P(C)(C)=O)n2)c(OC)cc1N1CCC(N2CCN(C)CC2)CC1]

### Step 1: Synthesis of Compound 73-2

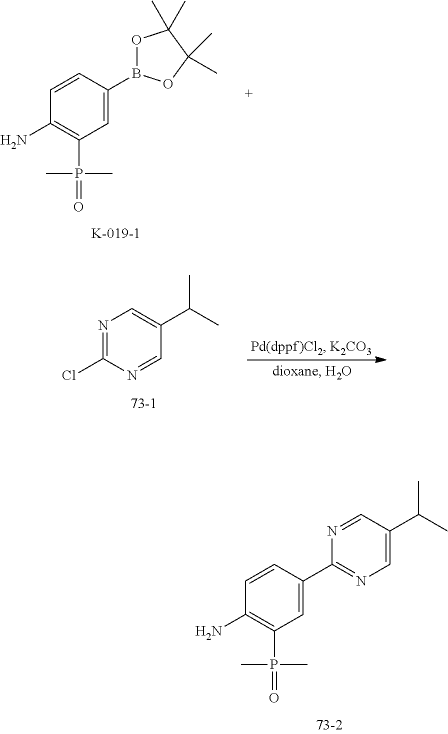

The compound 73-2 was synthesized as the method described for compound K-019-2 using compound 73-1 instead of raw compound 2-Bromothiazole. For compound 73-2, MS: 290.3 [M+H]⁺

### Step 2: Synthesis of Compound 73-3

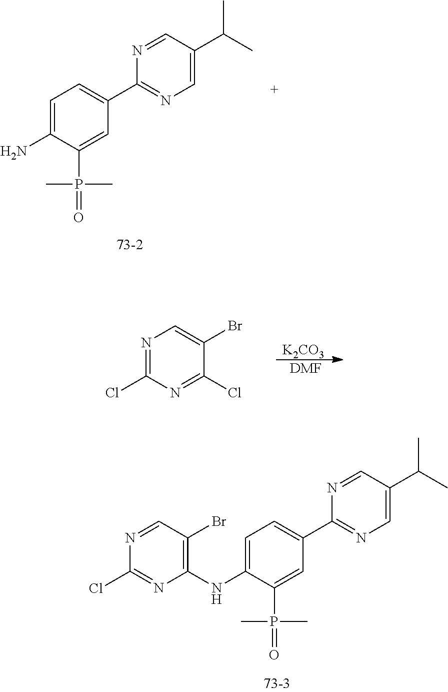

The compound 73-3 was synthesized as the method described for compound 1-10 using compound 73-2 instead of raw compound 1-8. For compound 73-3, MS: 480.03 [M+H]⁺

### Step 3: Synthesis of 73

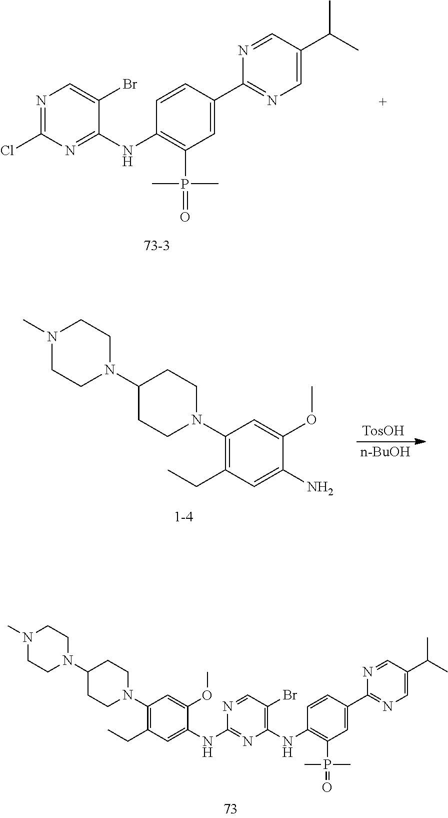

The compound 73 was synthesized as the method described for compound 1 using compound 73-3 instead of raw compound 1-10. For compound 73, MS: 776.31 [M+H]⁺

### Example 180: Synthesis of Compound 74

### (2-((5-Bromo-2-((5-ethyl-2-methoxy-4-(4-(4-methylpiperazin-1-yl)piperidin-1-yl)phenyl)amino)pyrimidin-4-yl)amino)-5-(2-(dimethylamino)pyrimidin-5-yl)phenyl)dimethylphosphine oxide

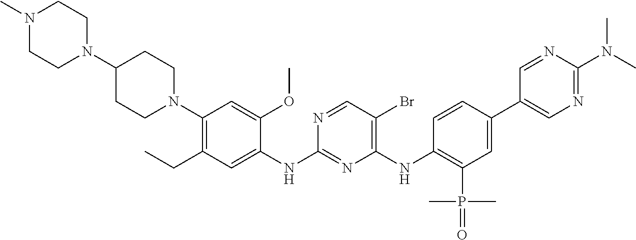

### Step 1: Synthesis of Compound 74-2

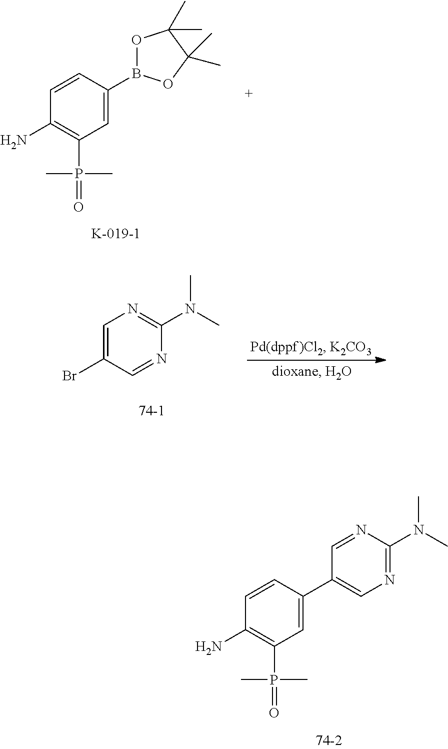

The compound 74-2 was synthesized as the method described for compound 5-3 using compound 74-1 instead of raw compound 5-2. For Compound 74-2, MS: 291.13 [M+H]⁺

### Step 2: Synthesis of Compound 74-3

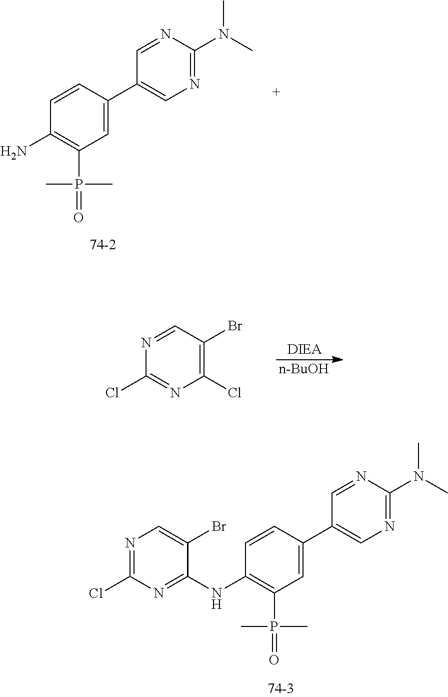

The compound 74-3 was synthesized as the method described for compound 1-10 using compound 74-2 instead of raw compound 1-8. For compound 74-3, MS: 481.02 [M+H]⁺

### Step 3: Synthesis of Compound 74

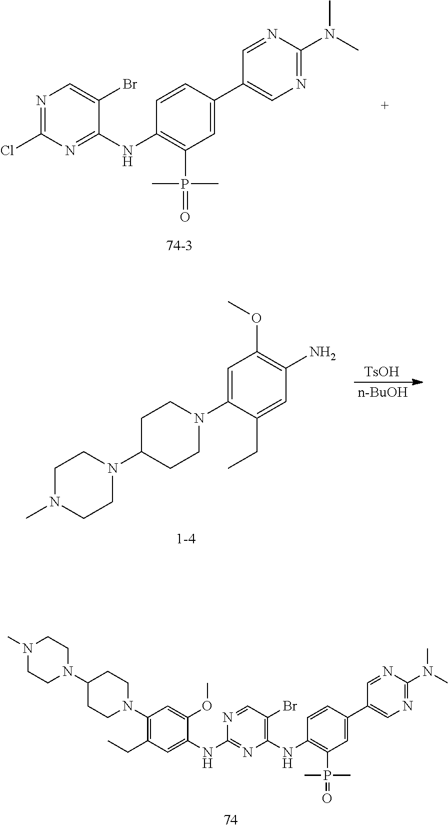

The compound 74 was synthesized as the method described for compound 1 using compound 74-3 instead of raw compound 1-10. For compound 74, MS: 777.30 [M+H]⁺

### Example 181: Synthesis of Compound 75

### (2-((5-Bromo-2-((5-ethyl-2-methoxy-4-(4-(4-methylpiperazin-1-yl)piperidin-1-yl)phenyl)amino)amino]pyrimidin-4-yl)amino)-5-(5-ethoxypyrimidin-2-yl)phenyl)dimethylphosphine oxide

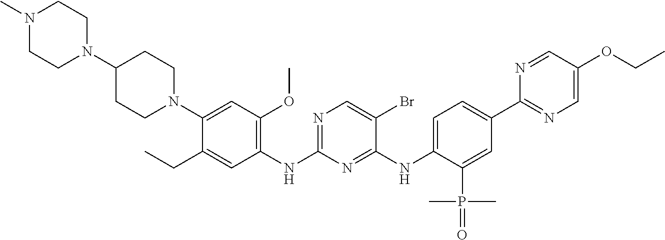 [SMILES: CCOc1cnc(-c2ccc(Nc3nc(Nc4cc(CC)c(N5CCC(N6CCN(C)CC6)CC5)cc4OC)ncc3Br)c(P(C)(C)=O)c2)nc1]

### Step 1: Synthesis of Compound 75-2

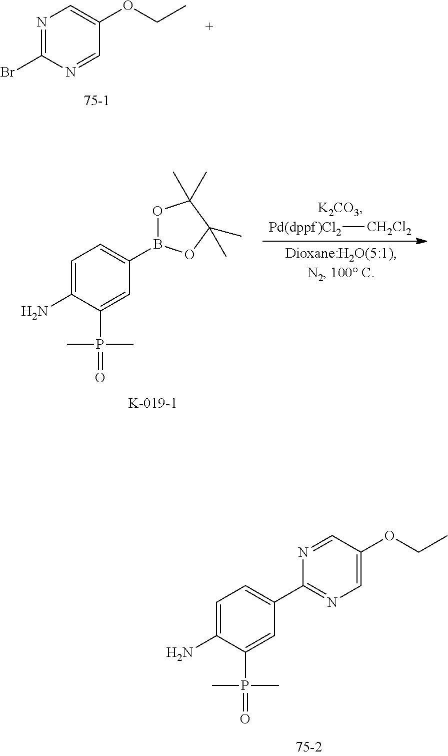

Add compound 75-1 (200 mg), K-019-1 (400 mg), K₂CO₃ (300 mg), Pd(dppf)Cl₂.CH₂Cl₂ (10 mg), dioxane (10 mL) and H₂O (2 mL) sequentially to the reaction flask, heat to 100° C. after mixing well under nitrogen protection, and stir with heating overnight. The reaction was complete as monitored by LCMS, and then stopped. The reaction mixture was poured to the water and stirred well, extracted with EtOAc, and washed with water, concentrated to remove solvent, and purified by column chromatography to obtain the desired product 75-2 (230 mg), MS: 292.11 [M+H]⁺.

### Step 2: Synthesis of Compound 75-3

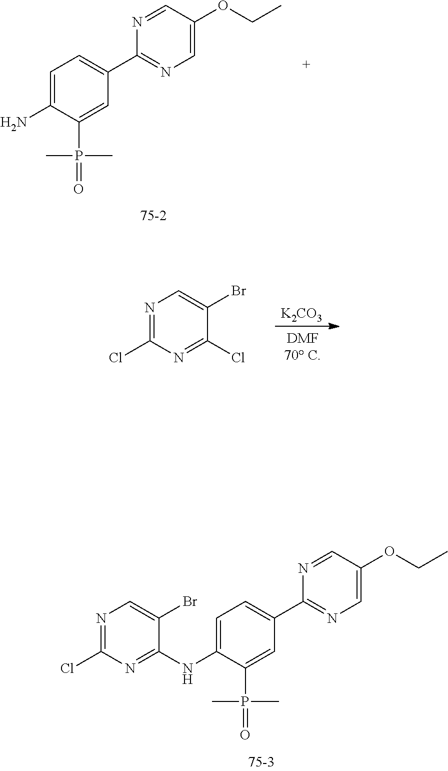

Add compound 75-2 (230 mg), 5-Bromo-2,4-dichloropyrimidine (200 mg), DMF (10 mL) and K₂CO₃ (150 mg) to the reaction flask, heat to 70° C. for reaction after well stirring. The reaction was complete as monitored by LCMS, and then stopped. The reaction solution was poured into water, mixed well, extracted with ethyl acetate, washed with water, and concentrated. After removing the solvent, it was purified with preparative TLC to obtain the desired compound 75-3 (64 mg), MS: 482.01 [M+H]⁺

### Step 3: Synthesis of Compound 75

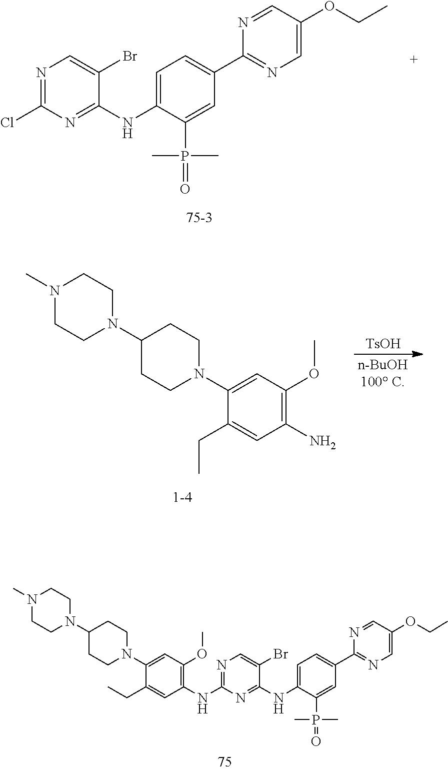

Add compounds 75-3 (64 mg), 1-4 (60 mg), TsOH (40 mg) and n-butanol (10 ml) successively to the reaction flask, mix well, heat to 100° C. and stir overnight. The reaction was complete as monitored by LCMS, and then stopped. After cooling, the solution was diluted with DCM, and quenched with water, the organic phase was separated, washed with saturated sodium bicarbonate, washed with water, dried, concentrated, and purified by preparative TLC to obtain the compound 75 (24 mg), MS: 778.29 [M+H]⁺.

### Example 182: Synthesis of Compound 76

### (2-((5-Bromo-2-((5-ethyl-2-methoxy-4-(4-(4-methylpiperazin-1-yl)piperidin-1-yl)phenyl)amino)amino]pyrimidin-4-yl)amino)-5-(2-ethoxypyrimidin-5-yl)phenyl)dimethylphosphine oxide

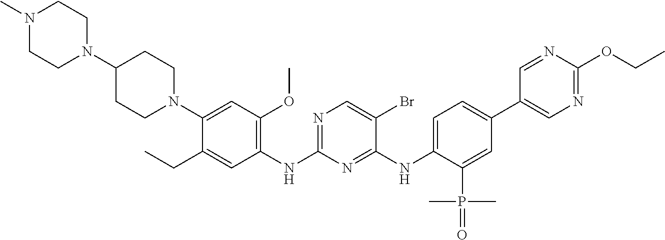 [SMILES: CCOc1ncc(-c2ccc(Nc3nc(Nc4cc(CC)c(N5CCC(N6CCN(C)CC6)CC5)cc4OC)ncc3Br)c(P(C)(C)=O)c2)cn1]

### Step 1: Synthesis of Compound 76-2

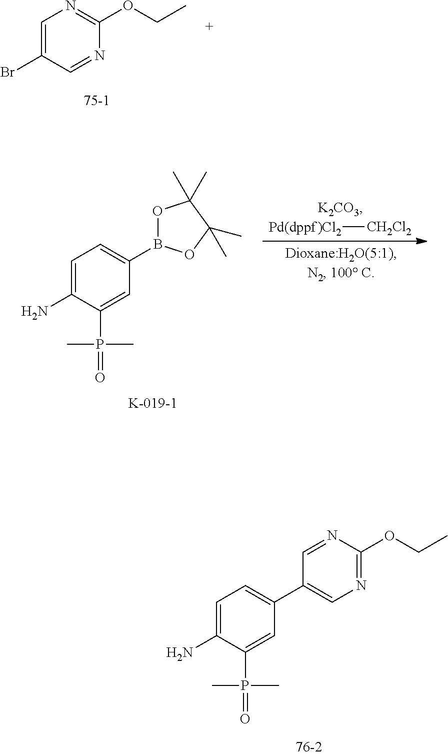

Add compound 76-1 (200 mg), K-019-1 (400 mg), K₂CO₃ (300 mg), Pd(dppf)Cl₂.CH₂Cl₂ (10 mg), dioxane (10 ml) and H₂O (2 ml) to the reaction flask, mix well, heat to 100° C. under nitrogen protection, and stir with heating overnight. The reaction was complete as monitored by LCMS, and then stopped. The reaction mixture was poured to the water and stirred well, extracted with EtOAc, and washed with water, concentrated to remove solvent, and purified by column chromatography to obtain the desired product 76-2 (280 mg), MS: 292.11 [M+H]⁺.

### Step 2: Synthesis of Compound 76-3

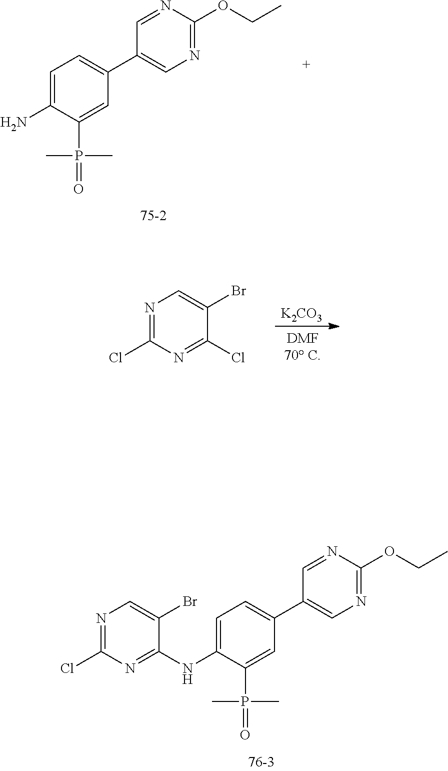

Add compound 76-2 (280 mg), 5-Bromo-2,4-dichloropyrimidine (200 mg), DMF (10 ml) and K₂CO₃ (150 mg) to the reaction flask, heat to 70° C. after mixing well. The reaction was complete as monitored by LCMS, and then stopped. The reaction mixture was poured to the water and stirred well, extracted with ethyl acetate, and washed with water, concentrated to remove solvent, and purified by preparation TLC to obtain the desired product 76-3 (67 mg), MS: 482.01 [M+H]⁺.

### Step 3: Synthesis of Compound 76

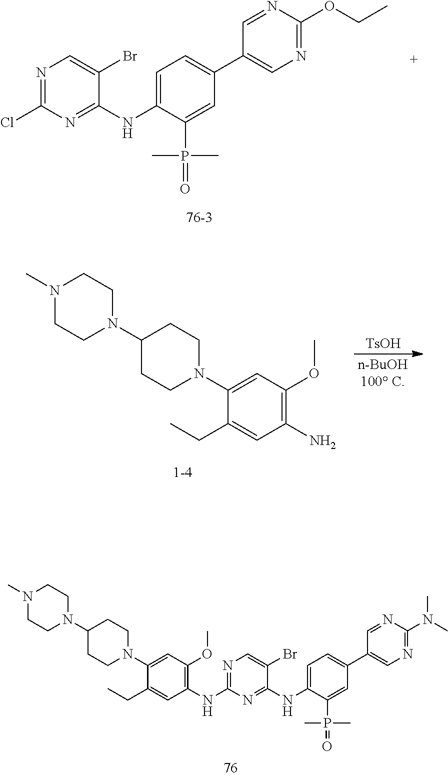

Add compound 76-3 (67 mg), 1-4 (60 mg). TsOH (40 mL) and n-butyl alcohol (10 mg) sequentially to the reaction flask, heat to 100° C. after mixing well and stir overnight. The reaction was complete as monitored by LCMS, and then stopped. After cooling, the solution was diluted with DCM, and quenched with water; the organic phase was separated, washed with saturated sodium bicarbonate and water, dried, concentrated, and purified by preparation TLC to obtain compound 76 (II mg), MS: 778.29 [M+H]⁺.

### Example 183: Synthesis of Compound 77

### (2-((5-Bromo-2-((5-ethyl-2-methoxy-4-(4-(4-methylpiperazin-1-yl)piperidin-1-yl)phenyl)amino]pyrimidin-4-yl)amino)-5-(6-(dimethylamino)pyridazin-3-yl)phenyl)dimethylphosphine oxide

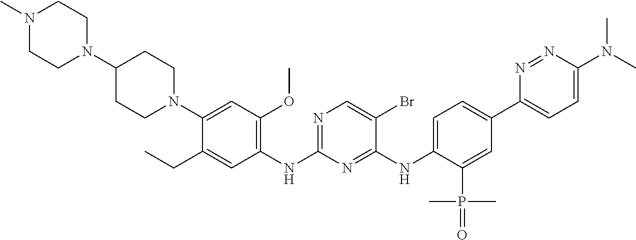

### Step 1: Synthesis of Compound 77-2

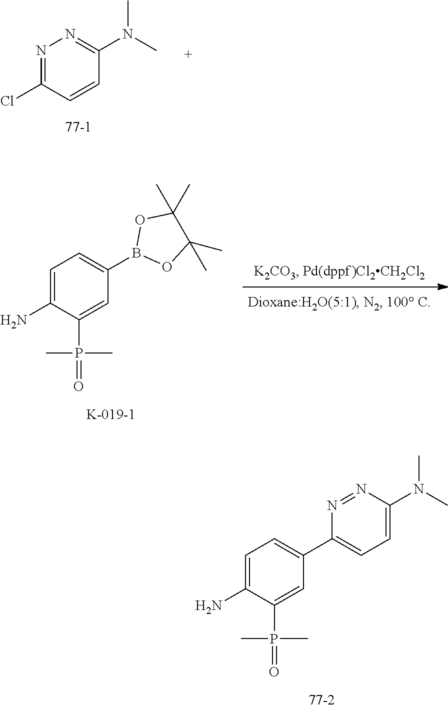

Add compound 77-1 (200 mg), K-019-1 (400 mg), K₂CO₃ (300 mg), Pd(dppf)Cl₂.CH₂Cl₂ (10 mg), dioxane (10 mL) and H₂O (2 mL) to the reaction flask, mix well, heat to 100° C. under nitrogen protection, and stir with heating overnight. The reaction was complete as monitored by LCMS, and then stopped. The reaction mixture was poured to the water and stirred well, extracted with EtOAc, washed with water, concentrated to remove solvent, and purified by column chromatography to obtain the desired product 77-2 (65 mg), MS: 291.13 [M+H]⁺.

### Step 2: Synthesis of Compound 77-3

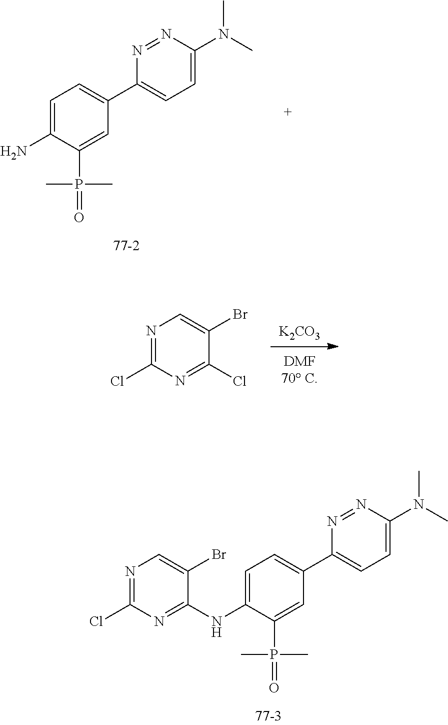

Add 77-2 (65 mg), 5-Bromo-2,4-dichloropyrimidine (50 mg), DMF (6 mL) and K₂CO₃ (50 mg) sequentially to the reaction flask, raise temperature to 70° C. for reaction after mixing well. The reaction was complete as monitored by LCMS, and then stopped. The reaction solution was poured into water and mixed well, extracted with ethyl acetate, washed with water, concentrated to remove the solvent, and purified by preparation TLC to obtain the desired compound 77-3 (54 mg), MS: 481.02 [M+H].

### Step 3: Synthesis of Compound 77

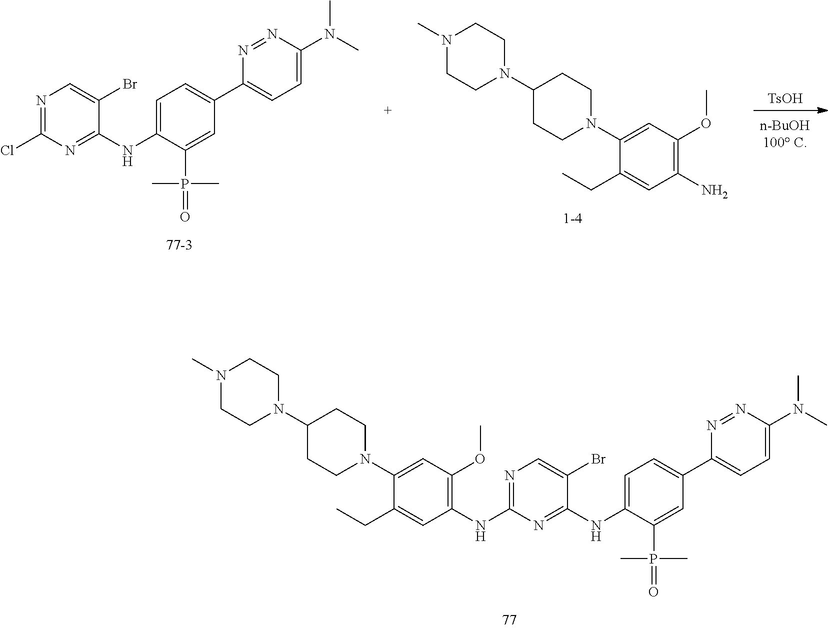

Add compounds 77-3 (54 mg), 1-4 (50 mg), TsOH (40 mg) and n-butanol (10 mL) sequentially to the reaction flask, and mix well, heat to 100° C. for stirring overnight. The reaction was complete as monitored by LCMS, and then stopped. After cooling, the solution was diluted with DCM, and quenched with water, the organic phase was separated, washed with saturated sodium bicarbonate and water, dried, concentrated and purified by preparation TLC to obtain compound 77 (40 mg), MS: 777.30 [M+H]⁺.

### Example 184: Synthesis of Compound 78

### (2-((5-Bromo-2-((5-ethyl-2-methoxy-4-(4-(4-methylpiperazin-1-yl)piperidin-1-yl)phenyl)amino)pyrimidin-4-yl)amino)-5-(pyrazin-2-yl)phenyl)dimethylphosphine oxide

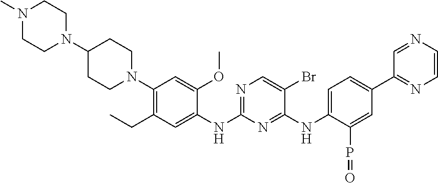 [SMILES: CCc1cc(Nc2ncc(Br)c(Nc3ccc(-c4cnccn4)cc3P=O)n2)c(OC)cc1N1CCC(N2CCN(C)CC2)CC1]

### Step 1: Synthesis of Compound 78-3

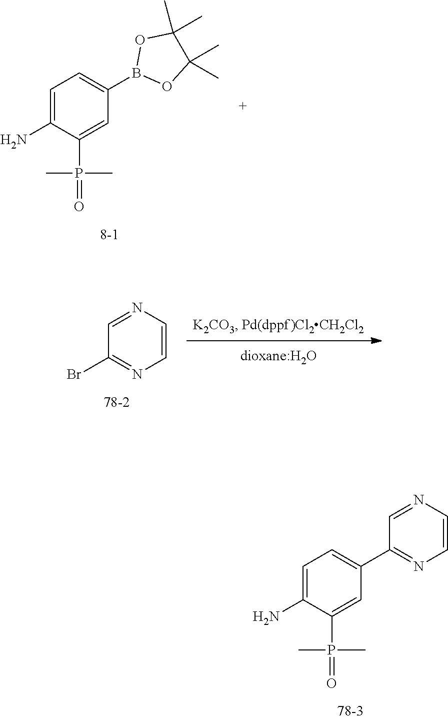 [SMILES: CC1(C)OB(c2ccc(N)c(P(C)(C)=O)c2)OC1(C)C ; Brc1cnccn1 ; CP(C)(=O)c1cc(-c2cnccn2)ccc1N]

Add 8-1 (300 mg), 78-2 (242 mg), Pd(dppf)Cl₂.CH₂Cl₂ (83 mg), K₂COⱼ(242 mg) and dioxane (6 mL) and H₂O (2 mL) to the reaction flask, and the gas in reactor was replaced with nitrogen for three times. Heat to 100° C., and stir with heating for 12 hr. The reaction was complete as monitored by LCMS, and then stopped. The excess solvent was removed by rotary evaporator. And then the solution was separated and purified by column chromatography (dichloromethane:methanol=15:1) to obtain the brown solid of desired product 78-3 (235 mg), MS: 248.09 [M+H]⁺

### Step 2: Synthesis of Compound 78-5

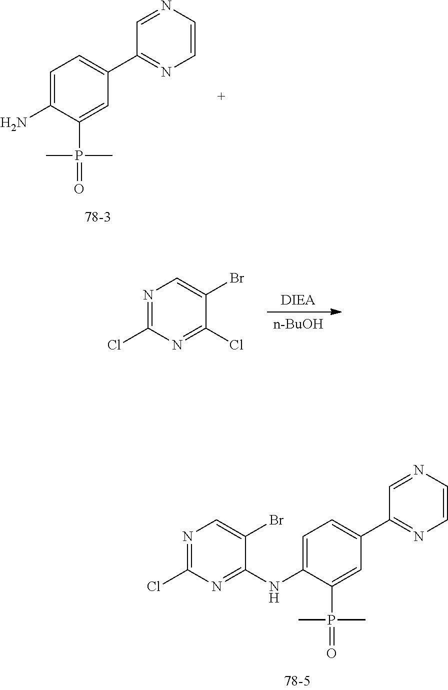

Add compound 78-3 (235 mg), 5-Bromo-2,4-dichloropyrimidine (65 mg), DIEA (614 mg) and n-BuOH (4 mL) to the reaction flask, heat to 110° C. and stir for 2 hr. The reaction was complete as monitored by LCMS, and then stopped. The excess solvent was removed by rotary evaporator. And then the solution was purified by preparation plate chromatography (dichloromethane:methanol=20:1) to obtain the pale brown solid of desired product compound 78-5 (93 mg), MS: 437.98 [M+H]⁺

### Step 3: Synthesis of Compound 78

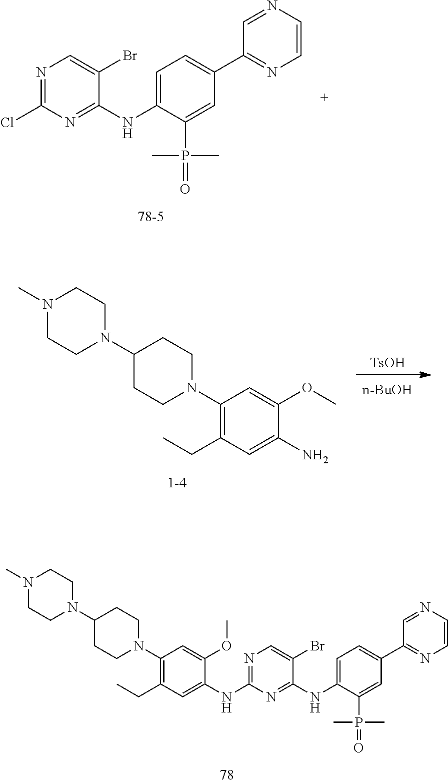

Add compounds 78-5 (93 mg), 1-4 (71 mg), p-toluenesulfonic acid (73 mg) and n-BuOH (4 mL) sequentially to the reaction flask, heat to 100° C. and stir with heating for 4 hr. The reaction was complete as monitored by LCMS, and then stopped. The water was added to the reaction mixture, which was extracted with dichloromethane (3×10 mL); the organic phase was washed with saturated brine (3×10 mL), and dried over anhydrous sodium sulfate; the solvent was removed, and the residuals was separated and purified by preparation panel chromatography (dichloromethane:methanol=15:1) to obtain brown solid of desired product compound 78 (117 mg), MS: 734.26 [M+H]⁺

### Example 185: Synthesis of Compound 79

### (2-((5-Bromo-2-((5-ethyl-2-methoxy-4-(4-(4-methylpiperazin-1-yl)piperidin-1-yl)phenyl)amino)pyrimidin-4-yl)amino)-5-(5-cyclopropylpyrazin-2-yl)phenyl)dimethylphosphine oxide

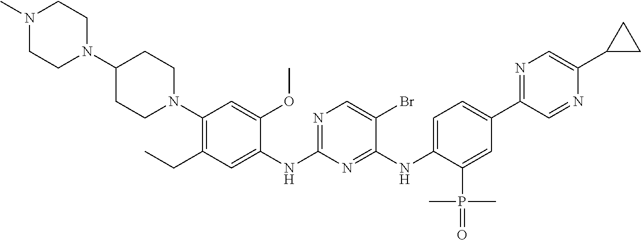 [SMILES: CCc1cc(Nc2ncc(Br)c(Nc3ccc(-c4cnc(C5CC5)cn4)cc3P(C)(C)=O)n2)c(OC)cc1N1CCC(N2CCN(C)CC2)CC1]

### Step 1: Synthesis of Compound 79-3

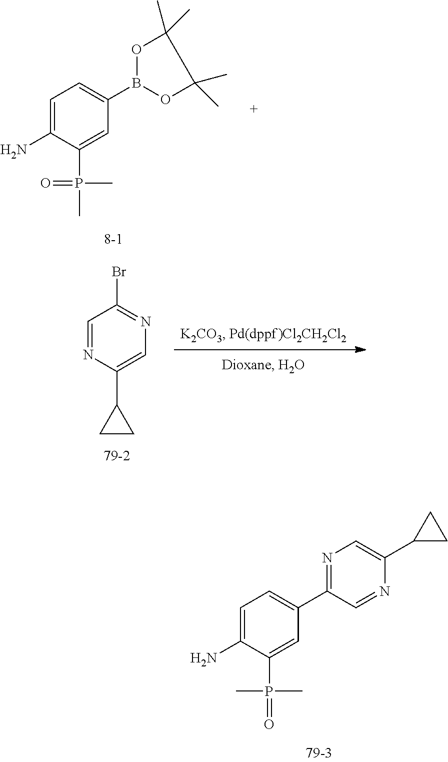 [SMILES: CC1(C)OB(c2ccc(N)c(P(C)(C)=O)c2)OC1(C)C ; Brc1cnc(C2CC2)cn1 ; CP(C)(=O)c1cc(-c2cnc(C3CC3)cn2)ccc1N]

Add 8-1 (300 mg), 79-2 (202 mg), K₂CO₃ (281 mg), Pd(dppf)Cl₂CH₂Cl₂ (83 mg), and dioxane (5 mL) and H₂O (2 mL) to the reaction flask; raise the temperature to 100° C. under nitrogen protection, stir with heating for 12 hr. The reaction was complete as monitored by LCMS, and then stopped. Column chromatography (dichloromethane:methanol=15:1) was used for separation and purification, and concentrated to afford the brown solid of desired product 79-3 (278 mg), MS: 288.12 [M+H]⁺

### Step 2: Synthesis of Compound 79-5

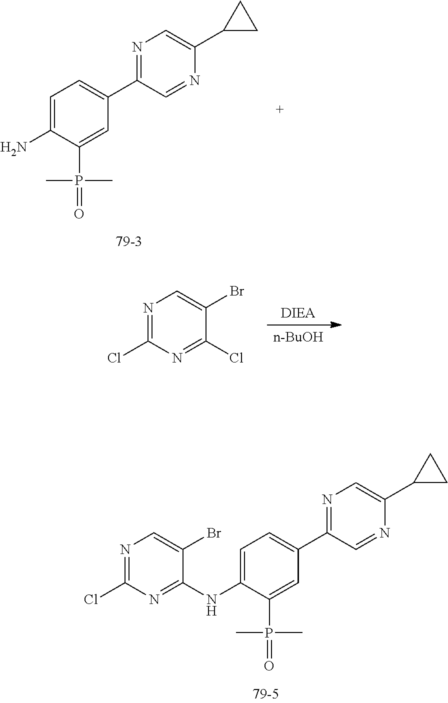

Add compound 79-3 (278 mg), 5-Bromo-2,4-dichloropyrimidine (1.1 g), DIEA (375 mg) and n-BuOH (8 mL) to the reaction flask successively; raise the temperature to 110° C. under nitrogen protection, stir with heating for 12 hr. The reaction was complete as monitored by LCMS, and then stopped. A solid was precipitated during the reaction. After filtration, the solid was washed with n-butanol and diethyl ether in turn, and dried by suction to obtain the brown solid of desired product compound 79-5 (328 mg), MS: 478.01 [M+H]⁺

### Step 3: Synthesis of Compound 79

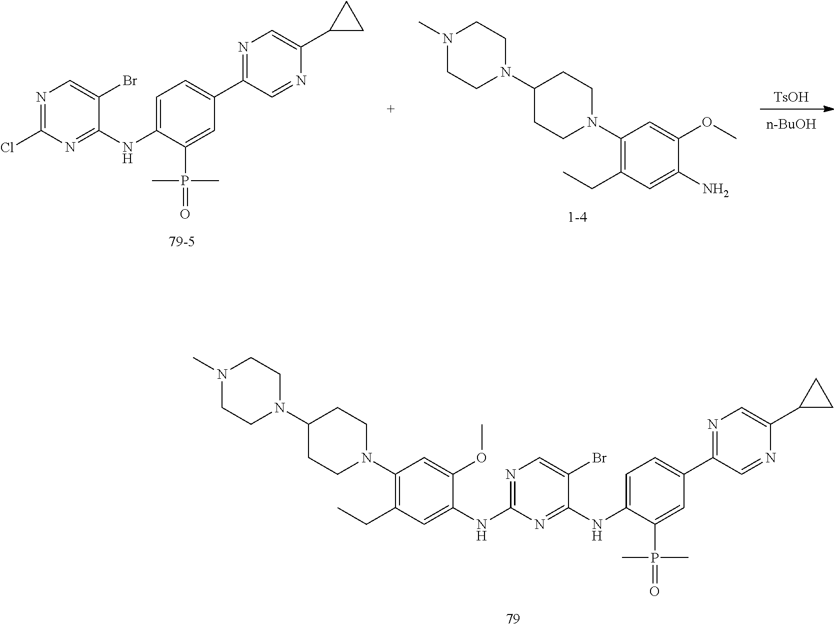

Add compounds 79-5 (328 mg), 1-4 (228 mg), p-toluenesulfonic acid (236 mg) and n-butanol (5 mL) to the reaction flask successively; heat to 100° C. under nitrogen protection, and stir for 12 hr. The reaction was complete as monitored by LCMS, and then stopped. The solution was adjusted to pH8 with Triethylamine added dropwise, and then concentrated. The residue was separated and purified with a thick preparation plate (dichloromethane:methanol=7:1), and the eluted product was concentrated to obtain an off-white solid compound 79 (111 mg), MS: 774.29 [M+H]⁺

### Example 186: Synthesis of Compound 80

### (2-((5-Bromo-2-((5-ethyl-2-methoxy-4-(4-(4-methylpiperazin-1-yl)piperidin-1-yl)phenyl)amino)pyrimidin-4-yl)amino)-5-(5-methylpyrazin-2-yl)phenyl)dimethylphosphine oxide

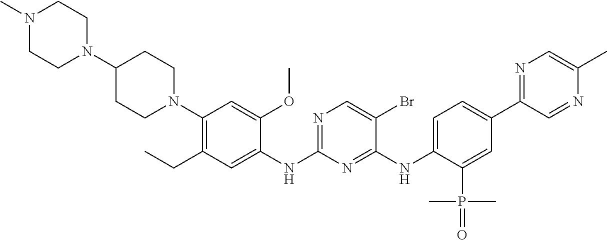

### Step 1: Synthesis of Compound 80-2

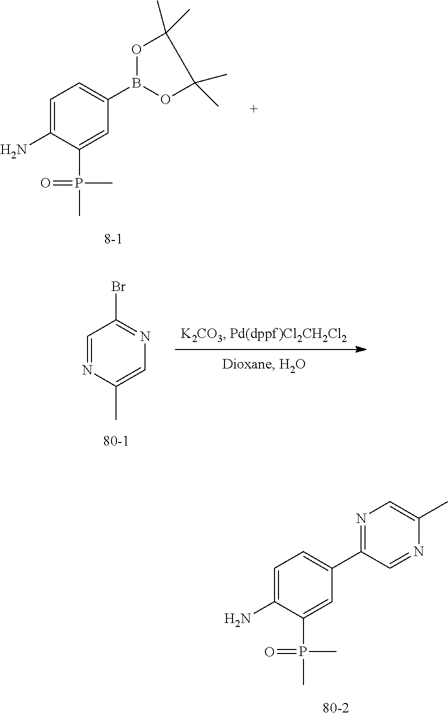

The compound 80-2 was synthesized as the method described for compound 79-3 using compound 80-1 (200 mg) instead of raw compound 79-2 to afford the solid powder compound 80-2 (134 mg). For compound 80-2, MS: 262.10 [M+H]⁺

### Step 2: Synthesis of Compound 80-3

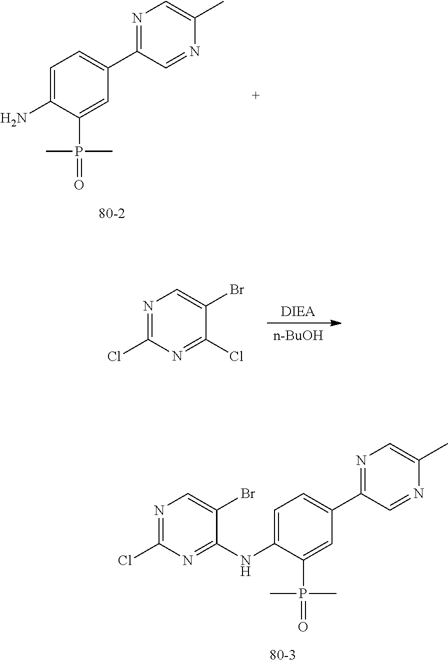

The compound 80-3 was synthesized as the method described for compound 1-10 using compound 80-2 (113 mg) instead of raw compound 1-8 to afford the solid powder compound 80-3 (220 mg). For compound 80-3, MS: 452.00 [M+H]⁺

### Step 2: Synthesis of Compound 80

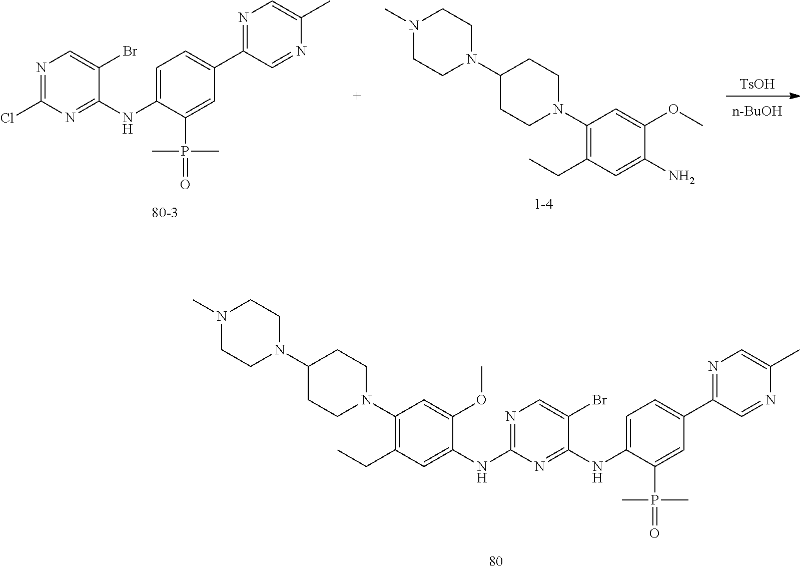

The compound 80 was synthesized as the method described for compound 1 using compound 80-3 (220 mg) instead of raw compound 1-10 to afford the solid powder compound 80 (65 mg). For compound 80, MS: 748.28 [M+H]⁺

### Example 187: Synthesis of Compound 81

### (2-((5-Bromo-2-((5-ethyl-2-methoxy-4-(4-(4-methylpiperazin-1-yl)piperidin-1-yl)phenyl)amino)pyrimidin-4-yl)amino)-5-(5,6-dimethylpyrazin-2-yl)phenyl)dimethylphosphine oxide

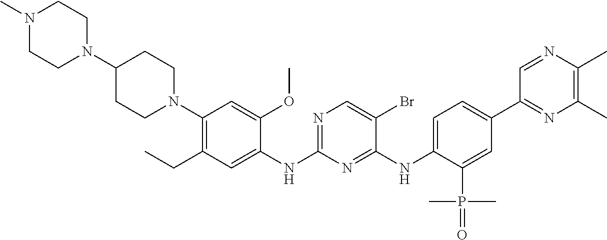

### Step 2: Synthesis of Compound 81-2

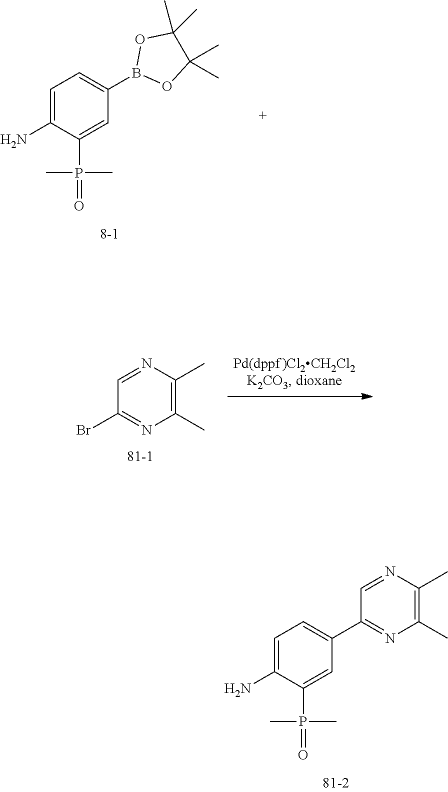

The compound 81-2 was synthesized as the method described for compound 79-3 using compound 81-1 instead of raw compound 79-2. For compound 81-2, MS: 276.12 [M+H]⁺

### Step 2: Synthesis of Compound 81-3

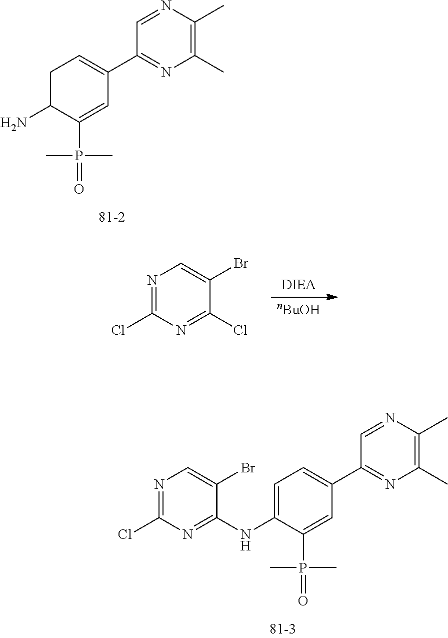

The compound 81-3 was synthesized as the method described for compound 1-10 using compound 81-2 instead of raw compound 1-2. For compound 81-3, MS: 466.01 [M+H]⁺

### Step 3: Synthesis of Compound 81

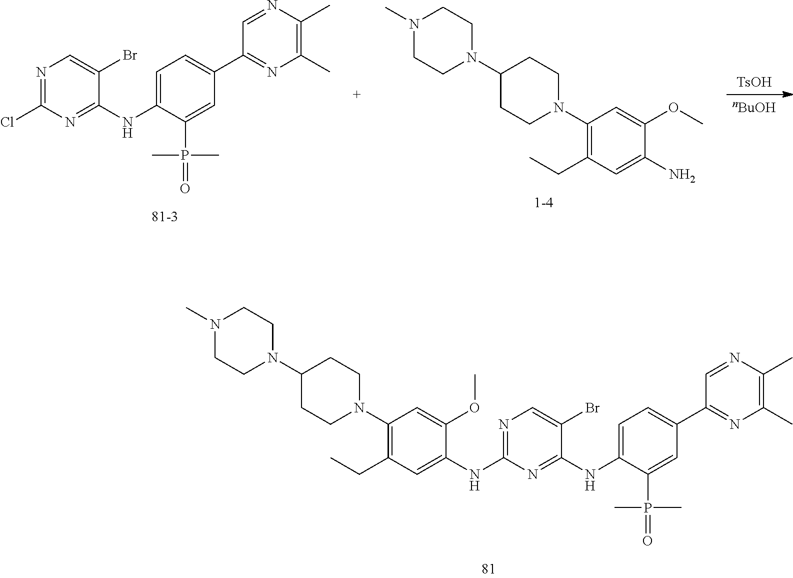

The compound 81 was synthesized as the method described for compound 1 using compound 81-3 instead of raw compound 1-10. For compound 81, MS: 762.29 [M+H]⁺

### Example 188: Synthesis of Compound 82

### (2-((5-Bromo-2-((5-ethyl-2-methoxy-4-(4-(4-methylpiperazin-1-yl)piperidin-1-yl)phenyl)amino)pyrimidin-4-yl)amino)-5-(6-methylpyridin-3-yl)phenyl)dimethylphosphine oxide

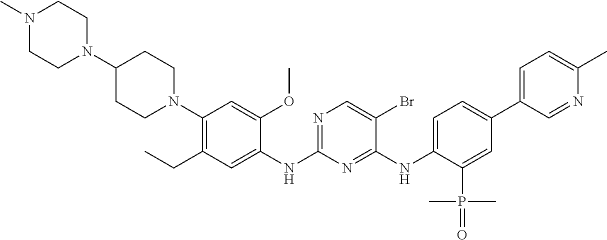

### Step 1: Synthesis of Compound 82-2

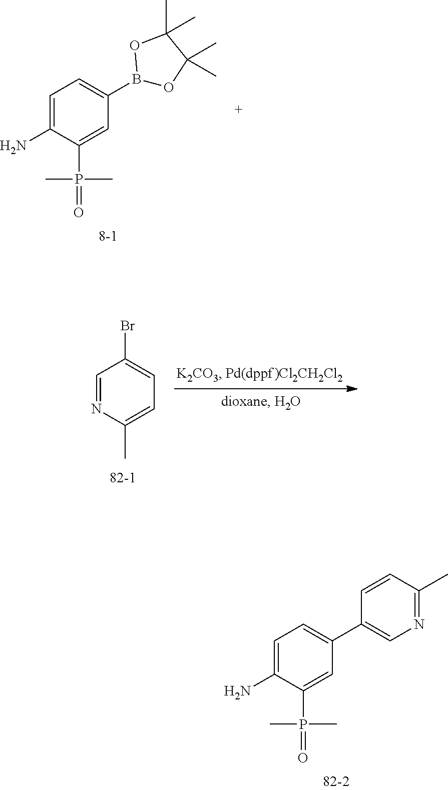

The compound 82-2 was synthesized as the method described for compound 79-3 using compound 82-1 (175 mg) instead of raw compound 79-2, to afford the solid powder compound 82-2 (227 mg). For compound 82-2, MS: 261.11 [M+H]⁺

### Step 2: Synthesis of Compound 82-3

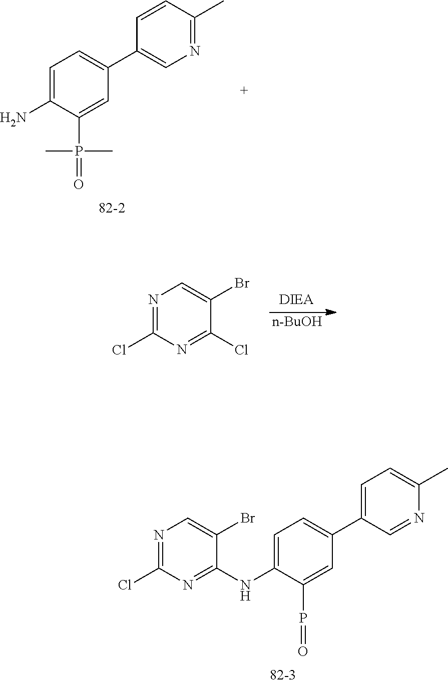

The compound 82-3 was synthesized as the method described for compound 1-10 using compound 82-2 (227 mg) instead of raw compound 1-8, to afford the solid powder compound 82-3 (300 mg). For compound 82-3, MS: 451.00 [M+H]⁺

### Step 3: Synthesis of Compound 82

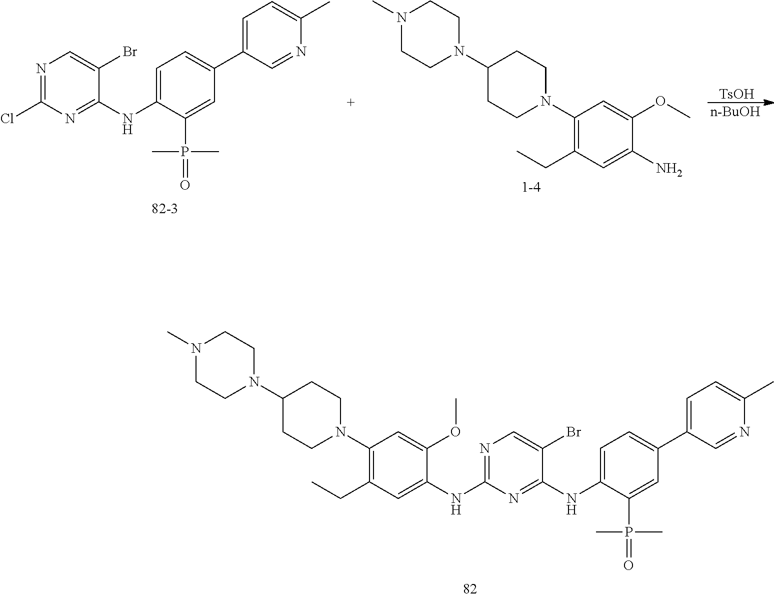

The compound 82 was synthesized as the method described for compound 1 using compound 82-3 (300 mg) instead of raw compound 1-10, to afford the brown powder compound 82 (38.5 mg). For compound 82, MS: 747.28 [M+H]⁺

### Example 189: Synthesis of Compound 83

### (2-((5-Bromo-2-((5-ethyl-2-methoxy-4-(4-(4-methylpiperazin-1-yl)piperidin-1-yl)phenyl)amino)pyrimidin-4-yl)amino)-5-(5-methylpyridin-2-yl)phenyl)dimethylphosphine oxide

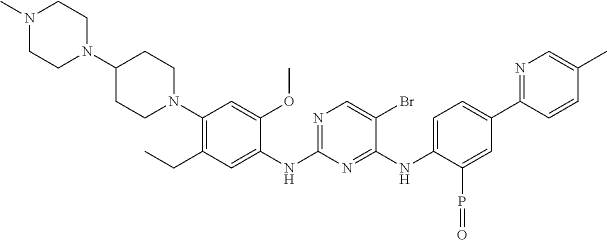

### Step 1: Synthesis of Compound 83-2

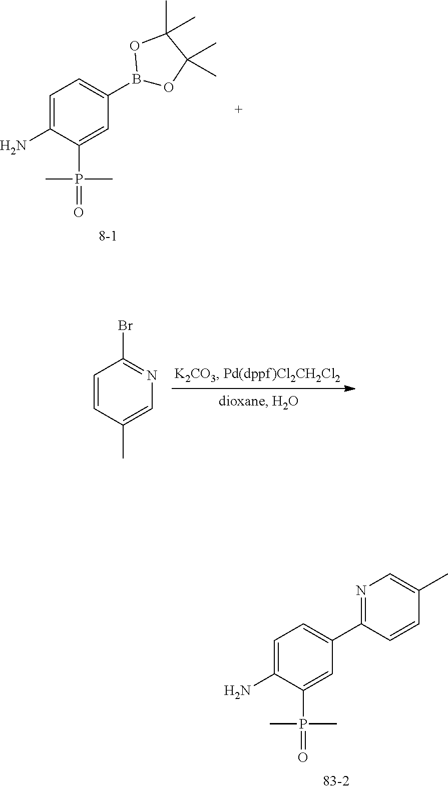

The compound 83-2 was synthesized as the method described for compound 79-3 using compound 2-Bromo-5-methylpyridine (175 mg) instead of raw compound 79-2, to afford the solid powder compound 83-2 (225 mg). For compound 83-2, MS: 261.11 [M+H]⁺

### Step 2: Synthesis of Compound 83-3

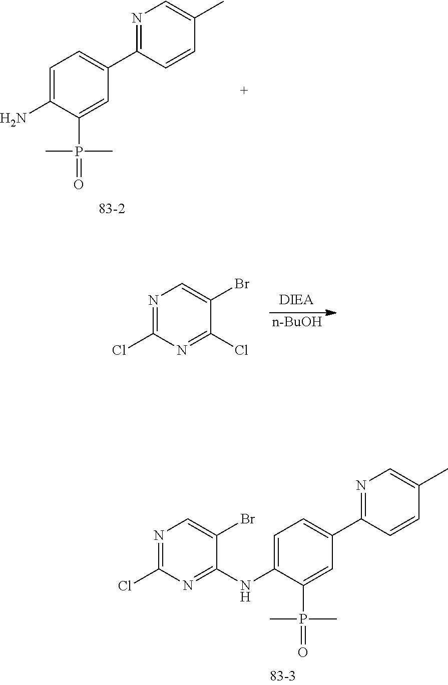

The compound 83-3 was synthesized as the method described for compound 1-10 using compound 83-2 (225 mg) instead of raw compound 1-8, to afford the solid powder compound 83-3 (300 mg). For compound 83-3, MS: 451.00 [M+H]⁺.

### Step 3: Synthesis of Compound 83

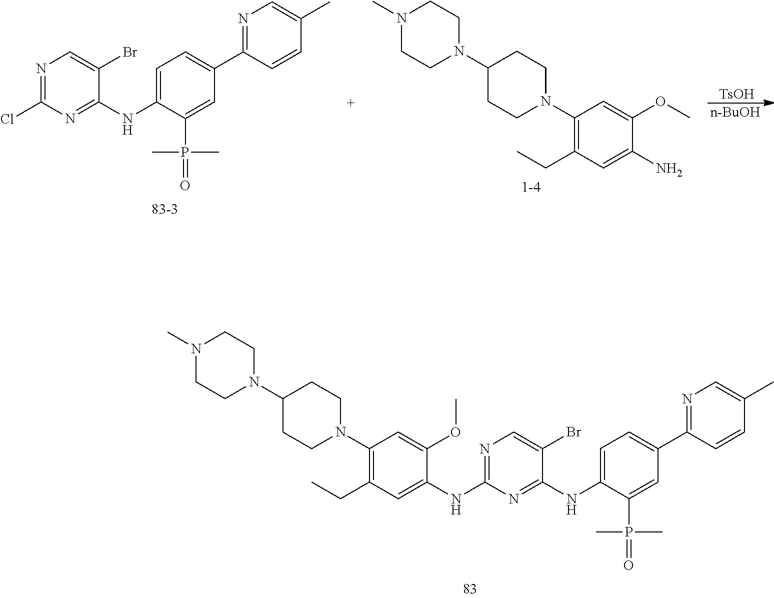

The compound 83 was synthesized as the method described for compound 1 using compound 83-3 (300 mg) instead of raw compound 1-10, to afford the brown powder compound 83 (54.5 mg). For compound 83, MS: 747.28 [M+H]⁺.

### Example 190: Synthesis of Compound 84

### (6-((5-Bromo-2-((5-ethyl-2-methoxy-4-(4-(4-methylpiperazin-1-yl)piperidin-1-yl)phenyl)amino)pyrimidin-4-yl)amino)-2-fluoro-3-(2-methyl-2H-1,2,3-triazol-4-yl)phenyl)dimethylphosphine oxide

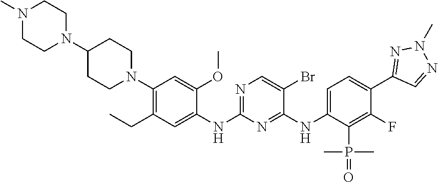

### Step 1: Synthesis of Compound 84-3

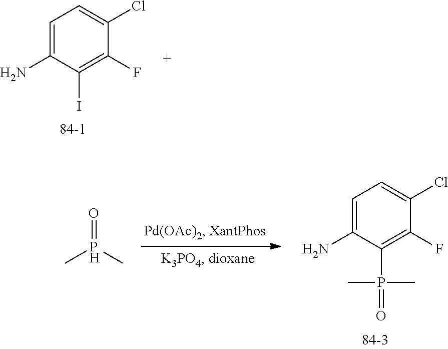

Add compound 84-1 (500 mg), dimethylphosphine oxide (216 mg), Pd(OAc)₂ (41 mg), XantPhos (107 mg), K₃PO₄ (586 mg) and dioxane (15 mL) to the reaction flask successively; the gas in the flask was replaced with nitrogen for three times; raise temperature to 110° C. and stir for 12 hr. The reaction was complete as monitored by LCMS, and then stopped. The excess solvent was removed by filtration and rotary evaporation; column chromatography (dichloromethane:methanol=20:1) was used for separation and purification to obtain the brown solid of desired product 84-3 (390 mg). For compound 84-3, MS: 222.02 [M+H]⁺

### Step 2: Synthesis of Compound 84-6

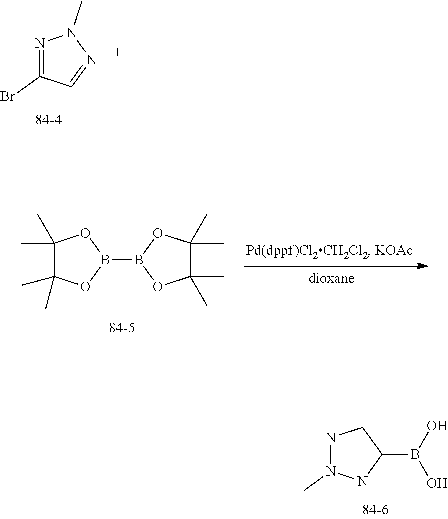

Add compounds 84-4 (500 mg), 84-5 (784 mg), Pd(dppf)Cl₂.CH₂Cl₂ (252 mg), KOAc (606 mg) and dioxane (15 mL) to the reaction flask successively; the gas in flask was replace with nitrogen for three times; heat to 100° C. and stir for 12 hr. The reaction was complete as monitored by LCMS, and then stopped. The excess solvent was removed by filtration and rotary evaporation; column chromatography (petroleum ether:ethyl acetate=6:1) was used for separation and purification to obtain the brown solid of desired product 84-6 (640 mg). For compound 84-6, MS: 128.06 [M+H]⁺

### Step 3: Synthesis of Compound 84-7

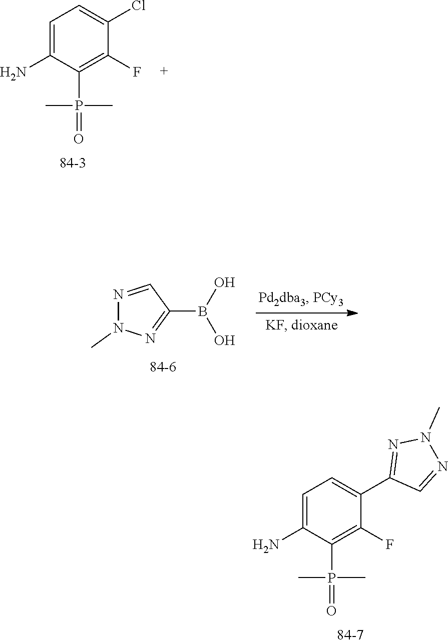

Add compounds 84-3 (159 mg), 84-6 (600 mg), Pd₂dba₃ (66 mg), PCy₃ (40 mg), KF (125 mg) and dioxane (10 ml) into the reaction flask successively; the gas in flask was replaced with nitrogen for three times; raise the temperature to 160° C. and carry out microwave reaction for 2.5 hr. The reaction was complete as monitored by LCMS, and then stopped. The excess solvent was removed by filtration and rotary evaporation; column chromatography (dichloromethane:methanol=30:1) was used for separation and purification to get the yellow solid of desired product 84-7 (320 mg). For compound 84-7, MS: 269.09 [M+H]⁺

### Step 4: Synthesis of Compound 84-8

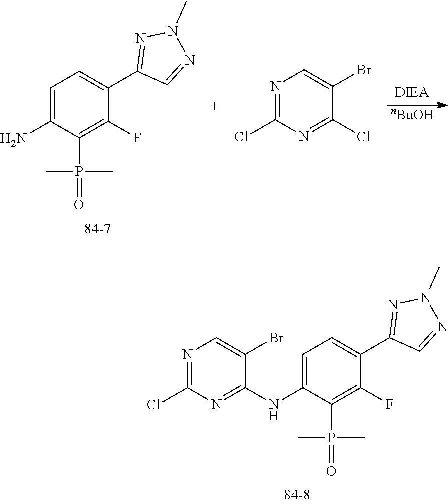

Compound 84-8 was synthesized as the method described for compound 1-10 using compound 84-7 instead of raw compound 1-8. For compound 84-8, MS: 458.98 [M+H]⁺

### Step 5: Synthesis of Compound 84

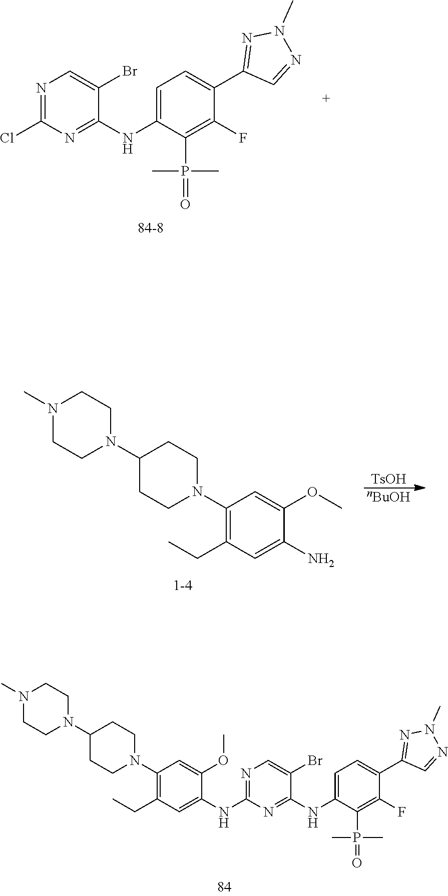

Compound 84-8 was synthesized as the method described for compound 1 using compound 84-8 instead of raw material 1-10. For compound 84, MS: 755.26 [M+H]⁺

Compounds of embodiments 191-192 were prepared as the synthetic method described in embodiment 84, but with substituted raw materials.

| | | | Molecular |
|---|---|---|---|
| | | | weight |
| Embodiment | Structural formula | Name | [M + H]⁺ |
| Embodiment 191 (compound 85) | 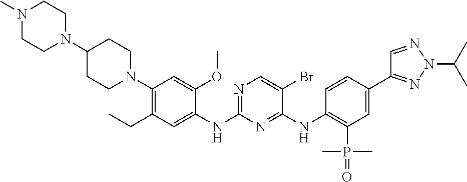 [SMILES: CCc1cc(Nc2ncc(Br)c(Nc3ccc(-c4cnn(C(C)C)n4)cc3P(C)(C)=O)n2)c(OC)cc1N1CCC(N2CCN(C)CC2)CC1] | (2-((5-Bromo-2-((5-ethyl-2-methoxy- 4-(4-(4-methylpiperazin-1-yl)piperidin- 1-yl)phenyl)amino)pyrimidin-4-yl) amino)-5-(2-isopropyl-2H-l,2,3-triazol- 4-yl)phenyl)dimethylphosphine oxide | 765.30 |
| Embodiment 192 (compound 86) | 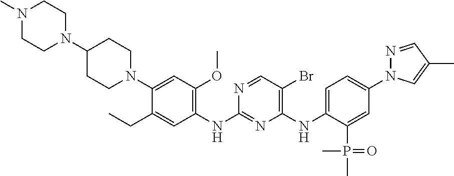 | (2-((5-Bromo-2-((5-ethyl-2-methoxy- 4-(4-(4-methylpiperazin-1-yl)piperidin- 1-yl)phenyl)amino)pyrimidin-4-yl) amino)-5-(4-methyl-1H-pyrazol-1-yl) phenyl)dimethylphosphine oxide | 736.28 |

### Pharmacological Testing

### Control Example

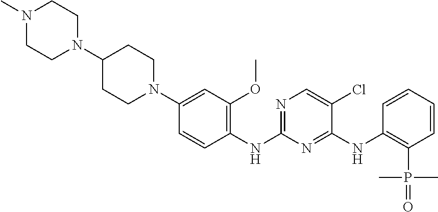

### Brigatinib

Prepare the compound thereof as described for compound 5 in WO2009143389A1,

### Comparative Compound 1

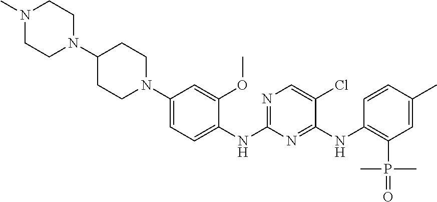

### Step 1: Synthesis of Compound 1-3

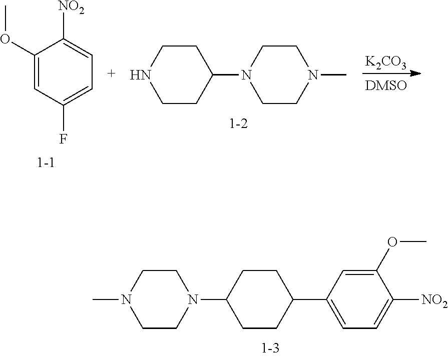

Add 1-1 (1.00 g), 1-2 (1.29 g), K₂CO₃ (1.62 g) and DMSO (10 mL) to the reaction flask successively and heat to 90° C. The mixture was stirred with heating for 12 hr. The reaction was complete as monitored by LCMS and stopped. The reaction solution was poured into water (50 mL), and extracted with DCM (2×30 mL); the organic phase was washed with water (3×20 ml) and saturated brine (20 mL), dried over anhydrous sodium sulfate and concentrated to get the crude product, which was slurried with ether (20 mL), to obtain the desired yellow solid product 1-3 (1.60 g), MS: 335.20 [M+H]⁺

### Step 2: Synthesis of Compound 1-3

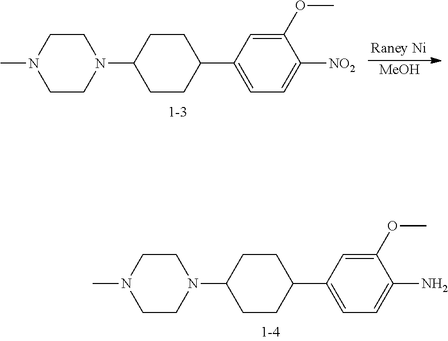

Add compound 1-3 (1.60 g), raney nickel (0.50 g) and MeOH (20 ml) to the reaction flask, introduce H₂ and stir the solution at room temperature for 3 hr. The reaction was complete as monitored by LCMS, and stopped. The solution was filtered with suction, and rinsed with methanol (20 mL); the organic phase was collected to remove the solvent and get the desired off-white solid compound 1-4 (1.45 g), MS: 305.23 [M+H]⁺

### Step 3: Synthesis of Compounds 1-7

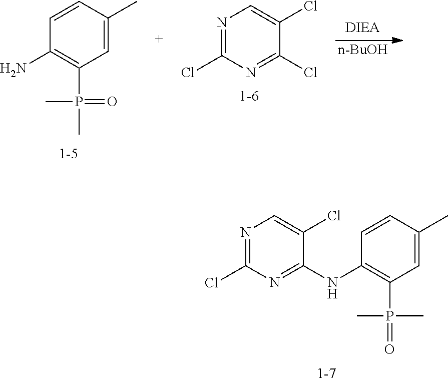

Add compounds 1-5 (0.5 g), compounds 1-6 (0.5 g), DIEA (1.06 g) and n-butyl alcohol (5 ml) to the reaction flask successively, and heat to 100° C. The mixture was heated and stirred for 3 hr. The reaction was complete as monitored by LCMS and stopped. After concentration, the reaction solution was separated and purified by column chromatography (dichloromethane:methanol=20:1), the solvent was removed to obtain the desired white solid product compound 1-7 (600 mg), MS: 330.03 [M+H]⁺

### Step 4: Synthesis of Comparative Compound 1

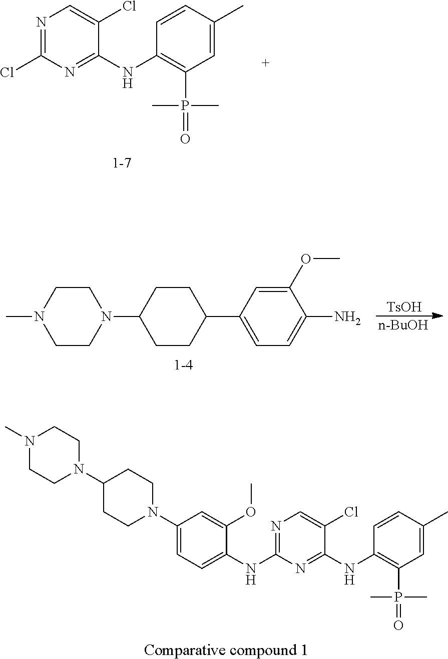

Add compounds 1-7 (100 mg), compounds 1-4 (92 mg), p-toluenesulfonic acid (104 mg) and n-butanol (6 ml) successively to the reaction flask, heat to 100° C. and stir for 5 hr. The reaction was complete as monitored by LCMS and stopped. The reaction solution was poured to sodium carbonate aqueous solution (15 ml), and extracted with dichloromethane (2×15 ml); the organic phase was washed with saturated brine (3×10 ml), dried with anhydrous sodium sulfate, separated and purified by column chromatography (dichloromethane:methanol=10:1), and concentrated to obtain the quasi white solid of comparative compound 1 (63 mg), MS: 598.27 [M+H]⁺

### Test 1 EGFR Δ19del/T790M/C797S Kinase Test

Mobility variation analysis was performed to determine the affinity of the compound for EGFRΔ19del/T790M/C797S, EGFR WT and IGF1R. The enzymatic reaction scheme is as follows:

1. Prepare 1*kinase buffer as follows.

| 1* kinase buffer | Final concentration |
|---|---|
| HEPES PH 7.5(mM) | 50 |
| Brij-35 | 0.0150% |
| DTT(mM) | 2 |
| Mgcl₂, Mncl₂(mM) | 10 |

2. Preparation of compound concentration gradient: The test compound was tested with an initial concentration of 3000 nM (300 nM for some triple mutations and 100 nM for others), diluted in a 384 source plate (96-well plate for comparative compound 1 and compounds 1-38) to a 100% DMSO solution with 100-fold final concentration, and the compound is 3-fold diluted with Precision to 10 concentrations. A dispenser Echo 550 was used to transfer 250 nL 100-fold final concentration of the compound to the target plate OptiPlate-384F.

3. Prepare 2.5-fold final concentration (5-fold for comparative compound 1 and compounds 1-38) of kinase solution with 1×Kinase buffer. Following steps should also be carried out for comparative compound 1 and compounds 1-38: Add each prepared intermediate dilution compounds to the compound wells of 384-well plate respectively, and test the repeated wells of each concentration; add 5% DMSO of 5 plates into the negative control well and the positive control well respectively. Prepare 2.5-fold final concentration of kinase solution with 1×Kinase buffer.

4. Add 10 μL of 2.5-fold final concentration of kinase solution to the compound well and the positive control well; add 10 μL of 1×Kinase buffer to the negative control well.

5. Centrifuge at 1000 rpm for 30 s, shake the reaction plate for well mixing, and then incubate at room temperature for 10 minutes.

6. Prepare a mixture of ATP and Kinase substrate at 5/3-fold final concentration with 1×Kinase buffer.

7. Add 15 μL mixture of 5/3-fold (2.5-fold for comparative compound 1 and compounds 1-38) final concentration of ATP and substrate to start reaction.

8. Centrifuge the 384-well plate at 1000 rpm for 30 s, shake it well and then incubate it at room temperature for corresponding time.

9. Add 30 μL of stop detection solution to stop the kinase reaction, centrifuge at 1000 rpm for 30 s, and shake it well.

10. Read conversion rate with Caliper EZ Reader.

11. Calculation formula

% Inhibition=((Conversion%_max−Conversion%_sample)/(Conversion%_max−Conversion%_ₘᵢₙ))*100

Wherein: Conversion%_sample is the sample conversion rate reading; Conversion%_min: Mean value of negative control wells, representing the conversion rate reading of wells without enzymatic activity; Conversion%_max: Mean value of positive control wells, representing the conversion rate reading of wells without compound inhibition.

The dose-effect curve was fitted with concentration log value as X-axis and percentage inhibition rate as Y-axis. GraphPad Prism 5 analysis software was used for fitting Log (inhibitor) vs. response—Variable slope with the dose-effect curve to obtain IC50 value of enzymatic activity for each compound.

The calculation formula is as Y-Bottom+(Top-Bottom)/(1+10\hat{ }((Log IC₅₀−X)*HillSlope)).

The results are expressed as IC₅₀ values, as shown in Table 1.

TABLE 1

| | EGFRΔ19del/ | | |
|---|---|---|---|
| | T790M/C797S | EGFR WT | EGFR IGF1R |
| Compound | IC₅₀(nM) | IC₅₀(nM) | IC₅₀(nM) |
| Brigatinib | 0.7 | 1.4 | 23.4 |
| K-002 | 0.2 | 11.5 | 124.0 |
| K-004 | 0.3 | 28.2 | 202 |
| K-006 | 0.5 | 17 | 56 |
| K-007 | 0.1 | 5.2 | 54.9 |
| K-008 | 0.24 | 15.0 | 120.0 |
| K-009 | 0.23 | 21.0 | 165.0 |
| K-010 | 0.4 | 17 | 46 |
| K-011 | 0.45 | 28 | 104 |
| K-012 | 0.19 | 12 | 35 |
| K-013 | 0.3 | 22.1 | 166.1 |
| K-014 | 0.5 | 32.5 | 50.5 |
| K-015 | 0.2 | 27.4 | 56.6 |
| K-016 | 0.5 | 13.1 | 94.0 |
| K-017 | 0.5 | 14.4 | 39.1 |
| K-018 | 0.2 | 17.1 | 67.2 |
| K-019 | 0.2 | 14.7 | 33.1 |
| K-020 | 0.3 | 22.0 | 126.3 |
| K-021 | 0.3 | 8.4 | 21.55 |
| K-022 | 0.2 | 31.8 | 52.5 |
| K-023 | 0.2 | 41.5 | 135.7 |
| K-024 | 0.3 | 35.0 | 71.1 |
| K-025 | 0.23 | 39 | 69 |
| K-026 | 0.6 | 187.9 | 412.8 |
| K-027 | 0.6 | 50.2 | 363.8 |
| K-028 | 0.1 | 26.0 | 39.1 |
| K-029 | 0.3 | 60.6 | 91.0 |
| K-030 | 0.2 | 7.8 | 21.5 |
| K-031 | 1.4 | 1128 | 394 |
| K-032 | 0.3 | 55.2 | 106.2 |
| K-033 | 0.3 | 55.6 | 120.7 |
| K-034 | 0.4 | 64.4 | 37.1 |
| K-035 | 0.3 | 42.8 | 48.7 |
| K-037 | 0.8 | 125 | 251 |
| K-038 | 0.2 | 19.2 | 56.1 |
| K-039 | 0.1 | 14.3 | 45.6 |
| K-040 | 0.3 | 24.5 | 134 |
| K-041 | 0.4 | 12.2 | 14.7 |
| K-042 | 0.4 | 15.9 | 71.0 |
| K-043 | 0.5 | 14.2 | 30.2 |
| K-044 | 0.3 | 8.2 | 38.2 |
| K-046 | 0.5 | 9.9 | 28.7 |
| K-047 | 0.8 | 43.0 | 216.6 |
| K-048 | 1.0 | 77.2 | 586.7 |
| K-049 | 0.8 | 31.7 | 432.0 |
| K-050 | 1.1 | 12.3 | 121 |
| K-052 | 1.6 | 44 | 886 |
| K-054 | 0.2 | 32 | 268 |
| K-056 | 0.9 | 38 | 223 |
| K-057 | 0.3 | 24 | 186 |
| K-058 | 0.5 | 16 | 91 |
| K-059 | 0.3 | 36 | 114 |
| K-060 | 0.4 | 13.9 | 196 |
| K-061 | 3.7 | 65 | 274 |
| K-062 | 0.2 | 18 | 126 |
| K-063 | 0.2 | 12.6 | 94 |
| K-064 | 0.4 | 18.1 | 191 |
| K-065 | 0.2 | 12.8 | 81 |
| K-066 | 1.1 | 13.9 | 134.2 |
| K-067 | 0.6 | 12.6 | 142.4 |
| K-069 | 0.67 | 26.71 | 219 |
| K-070 | 0.5 | 29.1 | 179 |
| K-071 | 0.3 | 29.4 | 180 |
| K-072 | 0.96 | 12.0 | 150.0 |
| K-073 | 0.3 | 22.2 | 127.3 |
| K-074 | 0.4 | 35.8 | 253 |
| K-078 | 0.8 | 31.7 | 432.0 |
| K-079 | 0.5 | 149.4 | 997 |
| K-087 | 0.1 | 17.2 | 96.1 |
| K-088 | 0.3 | 31 | 170 |
| K-089 | 0.3 | 169 | 259 |
| K-090 | 0.3 | 28.2 | 202 |
| K-091 | 0.8 | 38.8 | 279 |
| K-092 | 0.8 | 83.6 | 427.1 |
| K-093 | 1.0 | 77.2 | 586.7 |
| K-094 | 0.3 | 24.5 | 134 |
| K-101 | 1.1 | 13.9 | 134.2 |
| K-102 | 0.3 | — | — |
| K-104 | 0.4 | 23.1 | 378 |
| K-105 | 0.8 | 43.0 | 216.6 |
| K-106 | 0.5 | 17 | 56 |
| Comparative | 2.7 | 60 | 44.08 |
| compound 1 | | | |
| 1 | 0.5 | 10.3 | 91.5 |
| 2 | 0.7 | — | 150 |
| 3 | 0.3 | — | 53.7 |
| 4 | 0.6 | — | 71.9 |
| 5 | 0.4 | 12.5 | 117.2 |
| 6 | 0.9 | 16.7 | 146.9 |
| 7 | 0.5 | — | 187.2 |
| 8 | 0.4 | 10.8 | 67.9 |
| 9 | 0.4 | 63.4 | 826 |
| 10 | 0.6 | 19.5 | 109.4 |
| 11 | 0.6 | 28.6 | 494 |
| 12 | 1.1 | 13 | 309.9 |
| 13 | 0.4 | 31.1 | 182 |
| 14 | 0.4 | 13.9 | 149.3 |
| 15 | 0.3 | — | 66.5 |
| 16 | 0.4 | — | — |
| 17 | 0.7 | 20.1 | 363 |
| 18 | 0.7 | 20.3 | 125.6 |
| 19 | 0.6 | 22.0 | 144.2 |
| 20 | 0.5 | 19.0 | 120.0 |
| 21 | 0.7 | 25.6 | 224 |
| 22 | 0.3 | 40.8 | 120.2 |
| 23 | 0.5 | 18.9 | 261.5 |
| 24 | 0.7 | 66.9 | 263.8 |
| 25 | 0.4 | 37.6 | 169.1 |
| 26 | 0.4 | 18.9 | 101.9 |
| 27 | 0.3 | 15.6 | 72.0 |
| 28 | 0.2 | 12 | 61 |
| 29 | 0.2 | 16.0 | 80 |
| 30 | 0.2 | 20.7 | 118.2 |
| 31 | 0.3 | 27.6 | 178.9 |
| 32 | 0.4 | 19.3 | 184.1 |
| 33 | 0.3 | 27.3 | 177.0 |
| 34 | 0.2 | 29.7 | 91.1 |
| 35 | 0.5 | 21.5 | 238 |
| 36 | 0.2 | 35.5 | 219.4 |
| 37 | 0.3 | 28.8 | 263.3 |
| 38 | 0.3 | 11.1 | 64 |
| 39 | 0.4 | 18.3 | 138.4 |
| 41 | 1.5 | 58.9 | 748 |
| 42 | 1.24 | 86.2 | 1173 |
| 43 | 2.5 | 82.5 | 748 |
| 44 | 0.2 | 11.5 | 124.0 |
| 45 | 0.4 | 31.5 | 123.6 |
| 46 | 1.7 | 47.6 | 628.4 |
| 47 | 0.9 | 15.5 | 60.0 |
| 48 | 0.3 | — | 112.2 |
| 49 | 4.1 | 415.9 | 2785 |
| 50 | 0.2 | 32 | 268 |
| 51 | 1.3 | 30.8 | 268.9 |
| 52 | 1.9 | 203.0 | 1909.0 |
| 53 | 0.6 | 64.0 | 1096 |
| 54 | 0.6 | 42.2 | 624.2 |
| 55 | 1.5 | 36.4 | 497.3 |
| 56 | 2.4 | 72.5 | 886 |
| 57 | 0.4 | 13.9 | 196 |
| 58 | 3.7 | 65 | 274 |
| 59 | 1.3 | 41.0 | 394.6 |
| 60 | 0.6 | 47 | 418 |
| 61 | 1.6 | 44 | 886 |
| 62 | 0.8 | 216.2 | 992.2 |
| 63 | 1.3 | 38.8 | 334.7 |
| 64 | 0.8 | 43.1 | 330.0 |
| 68 | 0.9 | 84.7 | 663 |
| 69 | 4.0 | 315.8 | 1937 |
| 70 | 0.9 | 38.6 | 246.1 |
| 71 | 1.3 | 55.1 | 714.2 |
| 73 | 0.9 | 38 | 223 |
| 74 | 0.3 | 24 | 186 |
| 75 | 0.5 | 16 | 91 |
| 76 | 0.3 | 36 | 114 |
| 78 | 0.2 | 12.6 | 94 |
| 79 | 0.4 | 18.1 | 191 |
| 80 | 0.2 | 12.8 | 81 |
| 81 | 0.6 | 12.6 | 142.4 |
| 82 | 0.5 | 29.1 | 179 |
| 83 | 0.3 | 29.4 | 180 |
| 84 | 0.1 | — | 54.9 |
| 86 | 0.7 | 26.7 | 219 |

Notes:
“—” stands for “not tested”.

### Test 2 Ba/F3-Δ19del/T790M/C797S and Ba/F3-L858R/T790M/C797S Cell Proliferation Assay

1. Cell culture

Cell line: Ba/F3 cells with EGFR Δ19del/T790M/C797S mutation gene stably overexpressed named Ba/F3-Δ119del/T90M/C797S, and A431 and Ba/F3-EGFR wild-type cell line.

A. Culture medium

RPMI 1640 and 10% FBS and 1% PS; DMEM and 10% FBS and 1% PS.

B. Cell recovery

a) The medium was preheated in a 37° C. water bath in advance,

b) Remove the cryogenic vials from the liquid nitrogen tank, quickly put it into a 37° C. water bath, and completely melt it in 1 min.

c) Transfer the cell suspension to a 15 mL centrifuge tube containing 8 mL of medium, and centrifuge at 1000 rpm for 5 min.

d) Discard the supernatant, resuspend the cells in 1 mL culture medium, transfer it to a 75 cm² flask containing 15 mL of culture medium, and culture the cells in a incubator with 5% CO₂ at 37° C.;

C. Cell passage

a) The medium was preheated in a 37° C. water bath in advance.

b) Collect the cells in a 15 mL centrifuge tube and centrifuge at 1000 rpm for 5 min. Discard the supernatant, count to make the cell density at 1×10⁴ cells/mL, and then place it in a incubator with 5% CO₂ at 37 V,

2. Compound preparation

a) Dilute the test compound (20 mM stock solution) to 10 mM with 100% DMSO as the starting concentration, and then serially dilute 3 times with a “9+0” concentration in 96-well dilution plate (Cat #P-05525, Labcyte);

b) Dilute compound solution thereof to 1:100 (1:20 for comparative compound 1 and compounds 1-38) in medium to prepare 10-fold working solution;

3. Cell plate culture

a) Centrifuge the growth cells in logarithmic phase at 1000 rpm for 5 minutes, resuspend the cells in culture medium, and then count the cells;

b) Inoculate the cells into a 96-well cell culture plate with a density of 2000 cells/well;

4. Compound treatment

a) Compounds prepared at step 2 were added to cell plate with 15 μl per well, the final concentrations were 1000, 333, 111.1, 37, 12.3, 4.1, 1.4, 0.5, 0.2 and 0 nM, and the final concentration of DMSO was 0.1%. The blank control well was a culture medium (0.1% DMSO) (the blank control well of comparative compound 1 and compounds 1-38 was a culture medium (0.5% DMSO).

b) Incubate cells in an incubator for another 72 hours.

5. Assay

a) Take out the 96-well cell culture plate and add 50 μl CTG reagent (CellTiter Glo kit, promega, Cat #G7573);

b) Shake the plate for 2 minutes and let cool at room temperature for 10 minutes;

c) Read luminous signal value with PerkinElmer reader,

Analysis of Experimental Data

The data were analyzed by GraphPad Prism 6.0 software to obtain the fitting curve of compound activity.

Fitting compound IC50 from nonlinear regression equation:

Y=Min+(Max−Min)/(1+10\hat{ }((Log IC₅₀−X)*slope));

X: Logarithm of compound concentration; Y: luminous signal.

The result of cell proliferation assay is expressed by IC₅₀, as shown in Table 2.

TABLE 2

| | EGFR Δ19del/ | | |
|---|---|---|---|
| | T790M/C797S | EGFR WT | A431 |
| Compound | IC₅₀(nM) | IC₅₀(nM) | IC₅₀(nM) |
| Brigatinib | 64.88 | 22.50 | 309.2 |
| K-001 | 15.6 | 335 | 371.3 |
| K-002 | 9.5 | 166.5 | 2385 |
| K-004 | 18.42 | 467.3 | 2323 |
| K-005 | 27.9 | 482 | 415 |
| K-006 | 11.6 | 179.7 | 1271 |
| K-007 | 3.2 | 130 | 1440 |
| K-008 | 4.8 | 169 | 415 |
| K-009 | 16.4 | 268 | 2464 |
| K-010 | 7.2 | 52.8 | 692.9 |
| K-011 | 7.4 | 81.8 | 4452 |
| K-012 | 2.6 | 46 | 5218 |
| K-013 | 15.2 | 70 | 1420 |
| K-014 | 7.6 | 56 | 816 |
| K-015 | 13.6 | 178 | 953 |
| K-016 | 15.3 | 145 | 497 |
| K-017 | 12.0 | 445 | 812 |
| K-018 | 9.7 | 294.6 | 1062 |
| K-019 | 16 | 48.7 | 534.6 |
| K-020 | 9.2 | 226.2 | 1132 |
| K-021 | 2.3 | 84 | 3872 |
| K-022 | 16.5 | 281 | 1455 |
| K-023 | 5.0 | 89.5 | 1285 |
| K-024 | 15.6 | 171.8 | 600.8 |
| K-025 | 12.6 | 219.6 | >10000 |
| K-026 | 17.3 | 946 | >10000 |
| K-027 | 12.9 | 453 | 4048 |
| K-028 | 5.7 | 226.4 | 1244 |
| K-029 | 15.2 | 52.09 | 3181 |
| K-030 | 3.6 | 132.3 | 614.6 |
| K-031 | 16.8 | 150 | — |
| K-032 | 4.8 | 127.5 | 1335 |
| K-033 | 6.4 | 63.5 | 1590 |
| K-034 | 20.9 | 219.3 | 1226 |
| K-035 | 18.6 | 145.5 | 1678 |
| K-037 | 10.9 | 374 | 7534 |
| K-038 | 15.4 | 114.1 | 1042 |
| K-039 | 27.5 | 522.8 | >10000 |
| K-040 | 14.5 | 302 | 6628 |
| K-041 | 15.8 | 147 | 317 |
| K-042 | 4.3 | 206.4 | 815.4 |
| K-043 | 4.3 | 127 | 456.3 |
| K-044 | 3.8 | 165.6 | 280.1 |
| K-046 | 7.6 | 34.8 | 305.6 |
| K-047 | 29.8 | 95.6 | 12588 |
| K-048 | 12.9 | 156.2 | 23059 |
| K-049 | 38.4 | 373.4 | 1474 |
| K-050 | 20.6 | 217.5 | 1029 |
| K-051 | 1.4 | 15.8 | 125.2 |
| K-054 | 19.14 | 343.2 | 1510 |
| K-056 | 39.5 | 524 | 1527 |
| K-057 | 15.7 | 206 | 1562 |
| K-058 | 16.8 | 1005 | >10000 |
| K-059 | 37.4 | 352 | 1523 |
| K-060 | 40.8 | 293.1 | — |
| K-062 | 23.9 | 459 | 1800 |
| K-063 | 7.2 | 134.4 | 1090 |
| K-064 | 10.74 | 151.5 | 5647 |
| K-065 | 8.2 | 132.2 | 4612 |
| K-066 | 10.6 | 105 | 2297 |
| K-067 | 16.8 | 417 | 875 |
| K-068 | 28.6 | 1117 | 5772 |
| K-069 | 37.4 | 355.3 | 1613 |
| K-070 | 27.6 | 237.8 | 1623 |
| K-071 | 21 | 218 | 2537 |
| K-072 | 50.3 | 446 | 1926 |
| K-073 | 27.7 | 407.1 | 1510 |
| K-074 | 4.2 | 267.4 | 2761 |
| K-078 | 38.4 | 373.4 | 1474 |
| K-079 | 50.9 | 432 | 7648 |
| K-080 | 27.9 | 482 | 415 |
| K-081 | 1.0 | 52.1 | 379 |
| K-082 | 11.4 | 2965 | 1259 |
| K-083 | 1.0 | 113 | 910 |
| K-087 | 7.4 | 244.9 | 4819 |
| K-088 | 14.6 | 284.9 | >10000 |
| K-089 | 9.5 | 399.9 | >10000 |
| K-090 | 18.42 | 467.3 | 2323 |
| K-093 | 12.9 | 156.2 | 23059 |
| K-094 | 14.5 | 302 | 6628 |
| K-100 | 2.1 | — | 99.6 |
| K-101 | 10.6 | 105 | 2297 |
| K-104 | 64.2 | 204 | 3247 |
| K-105 | 29.8 | 95.6 | 12588 |
| K-106 | 11.6 | 179.7 | 1271 |
| Comparative | 49.9 | 836.4 | 597.5 |
| compound 1 | | | |
| 1 | 2.2 | 170.6 | 934.8 |
| 2 | 21.8 | — | 811.8 |
| 3 | 4.5 | 106.8 | 601.9 |
| 4 | 1.9 | 84.3 | 464.7 |
| 5 | 18.7 | — | >10000 |
| 6 | 21.4 | — | >10000 |
| 7 | 46.2 | — | — |
| 8 | 3.3 | 180.7 | 773 |
| 9 | 23.1 | — | 1453 |
| 10 | 9.7 | — | 14264 |
| 11 | 18.9 | — | 1023 |
| 12 | 10.7 | — | 989 |
| 13 | 40.5 | — | 1683 |
| 14 | 14.5 | — | 769.4 |
| 15 | 16.1 | — | — |
| 16 | 5.4 | 141.2 | 563.6 |
| 17 | 30.0 | — | 2203 |
| 18 | 13.2 | — | 1270 |
| 19 | 29.6 | — | 863.5 |
| 20 | 11.8 | 365.5 | 1470 |
| 21 | 8.8 | — | 1394 |
| 22 | 20.5 | — | 830.6 |
| 23 | 42.9 | — | — |
| 24 | 44.0 | — | — |
| 25 | 22.4 | — | 1696 |
| 26 | 5.1 | 162.1 | 1034 |
| 27 | 7.5 | 159.3 | 871 |
| 28 | 9.9 | 170.9 | 778 |
| 29 | 13.4 | 194.3 | 2665 |
| 30 | 15.2 | — | 8870 |
| 31 | 20.8 | 559 | 1033 |
| 32 | 17.8 | 195.8 | 748.8 |
| 33 | 13.5 | 436.9 | 962.2 |
| 34 | 5.8 | 180 | 5153 |
| 35 | 3.1 | 258.6 | >10000 |
| 36 | 22.3 | — | 4636 |
| 37 | 17.4 | — | >10000 |
| 38 | 11.2 | — | 711 |
| 44 | 9.5 | 166.5 | 2385 |
| 45 | 26.2 | — | 1075 |
| 46 | 30.3 | — | 2305 |
| 47 | 23.4 | — | 771.3 |
| 50 | 19.14 | — | 1510 |
| 51 | 23.5 | — | 577.9 |
| 55 | 35.0 | 1073 | 2959 |
| 57 | 40.8 | — | — |
| 59 | 26.3 | 1292 | 2296 |
| 63 | 24.5 | — | 512.2 |
| 64 | 47.3 | — | >10000 |
| 70 | 9.0 | 407 | 6291 |
| 72 | 29.4 | — | >10000 |
| 73 | 39.5 | — | 1527 |
| 74 | 15.7 | — | 1562 |
| 75 | 16.8 | 1005 | >10000 |
| 76 | 37.4 | — | 1523 |
| 77 | 27.2 | — | >10000 |
| 78 | 7.2 | 134.4 | 1090 |
| 79 | 10.74 | — | 5647 |
| 80 | 8.2 | — | 4612 |
| 81 | 16.8 | — | 875 |
| 82 | 27.6 | — | 1623 |
| 83 | 20.1 | — | 1430 |
| 84 | 5.8 | — | 1209 |
| 85 | 28.6 | 1117 | 5772 |
| 86 | 37.4 | — | 1613 |

Notes:
“—” stands for “not tested”.

1. A compound of Formula I, or a stereoisomer, tautomer, deuterated compound, pharmaceutically acceptable salt, prodrug, chelate, non-covalent complex or solvate thereof: 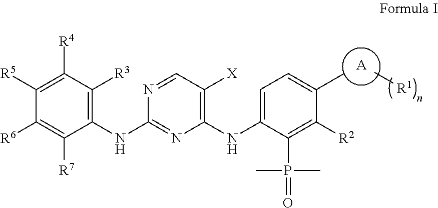
wherein,
X is halogen;
ring A is a 5- to 10-membered aromatic ring; the 5- to 10-membered aromatic ring is a 5- to 10-membered aryl or a 5- to 10-membered heteroaryl;
R¹ is selected from halogen, CN, OH, —NRR⁸, oxo, aryl, heteroaryl, —C₁₋₆ alkyl, haloalkyl, —C₁₋₆ alkoxy, —(CH₂)ₚ(O(CH₂)_{q})ᵣCH₃, —C₃₋₆ cycloalkyl, 3- to 6-membered heterocyclyl, —C₁₋₆ alkyl substituted with a 3- to 7-membered heterocyclyl and 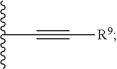
R² is selected from H, halogen, CN, NO₂, OH, —NRR⁸, —C₁₋₆ alkyl, —C₁₋₆ alkoxy, —(CH₂)ₚ(O(CH₂)_{q})ᵣCH₃, —C₃₋₆ cycloalkyl, 3- to 6-membered heterocyclyl; the —C₁₋₆ alkyl, —C₁₋₆ alkoxy, 3- to 6-membered heterocyclyl are optionally substituted or not substituted with halogen, OH, NH₂, aryl, heteroaryl, —C₁₋₆ alkyl, —C₃₋₆ cycloalkyl;
R, R⁸ and R⁹ are independently selected from H, CN, —C₁₋₆ alkyl, —C₃₋₆cycloalkyl and 3- to 6-membered heterocyclyl; wherein the —C₁₋₆ alkyl, —C₃₋₆ cycloalkyl and 3- to 6-membered heterocyclyl are optionally substituted or not substituted with halogen, OH, NH₂, aryl, heteroaryl, —C₁₋₆ alkyl, —C₃₋₆ cycloalkyl, 3- to 6-membered heterocyclyl;
R³, R⁴ and R⁷ are independently selected from H, halogen, CN, —NR¹⁰R¹¹, OH, —C₁₋₆ alkyl, —C₁₋₆ alkoxy, —(CH₂)ₚ(O(CH₂)_{q})ᵣCH₃, —C₃₋₆ cycloalkyl and 3- to 6-membered heterocyclyl; wherein the —C₁₋₆ alkyl, —C₁₋₆ alkoxy, —C₃₋₆ cycloalkyl and 3- to 6-membered heterocyclyl are optionally substituted or not substituted with halogen, OH, NH₂, —C₁₋₆ alkyl; and R³, R⁴ and R⁷ are not H at the same time;
R¹⁰ and R¹¹ are independently selected from H, —C₁₋₆ alkyl and —C₃₋₆ cycloalkyl;
R⁵ and R⁶ are independently selected from H, halogen, CN, NO₂, OH, —NR¹²R¹³, —C₁₋₆ alkyl, aryl, heteroaryl, —C₁₋₆ alkoxy, —C₃₋₆ cycloalkyl, 3- to 6-membered heterocyclyl, —(CH₂)ₛCH═CHR¹⁴; the —C₁₋₆ alkyl, aryl, heteroaryl, —C₁₋₆ alkoxy, —C₃₋₆ cycloalkyl and 3- to 6-membered heterocyclyl are optionally substituted or unsubstituted with halogen, OH, NH₂, —C₁₋₆ alkoxy, —C₃₋₆ cycloalkyl, substituted C₃₋₆ cycloalkyl, 3- to 6-membered heterocyclyl and substituted a 3- to 6-membered heterocyclyl, aryl, —C₁₋₆ alkanoyl and heteroaryl; and R⁵ and R⁶ are not H at the same time;
R¹², R¹³ and R¹⁴ are independently selected from H, —C₁₋₆ alkyl and —C₃₋₆ cycloalkyl;
n is 0, 1, 2, 3 or 4;
p is 1, 2 or 3;
q is 0, 1, 2 or 3;
r is 1, 2 or 3; and
s is 0, 1, 2 or 3.

2. (canceled)

3. The compound of claim 1, or a stereoisomer, tautomer, deuterated compound, pharmaceutically acceptable salt, prodrug, chelate, non-covalent complex or solvate thereof, wherein ring A is a 5- to 10-membered aryl or a 5- to 10-membered heteroaryl; wherein the 5- to 10-membered heteroaryl contains 1, 2 or 3 heteroatoms selected from N or S.

4. The compound of claim 1, or a stereoisomer, tautomer, deuterated compound, pharmaceutically acceptable salt, prodrug, chelate, non-covalent complex or solvate thereof, wherein ring A is

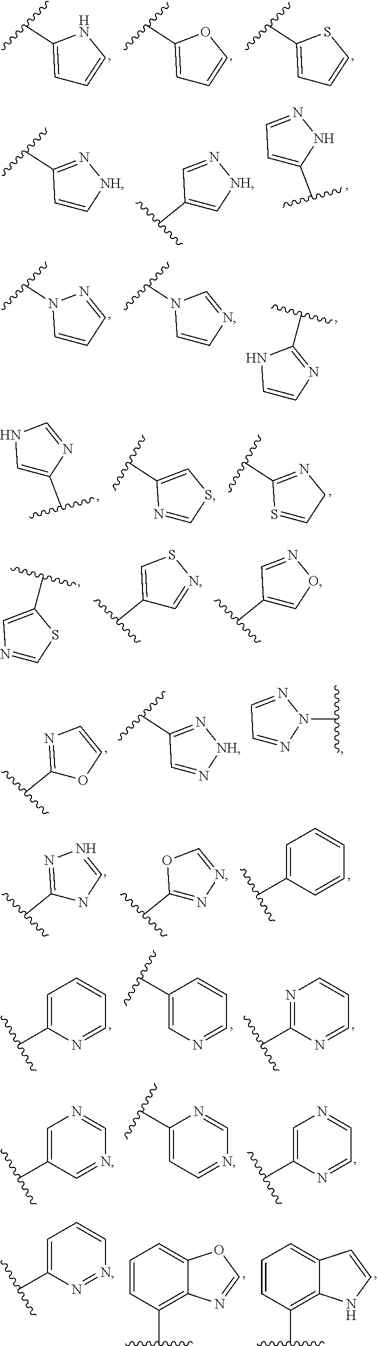

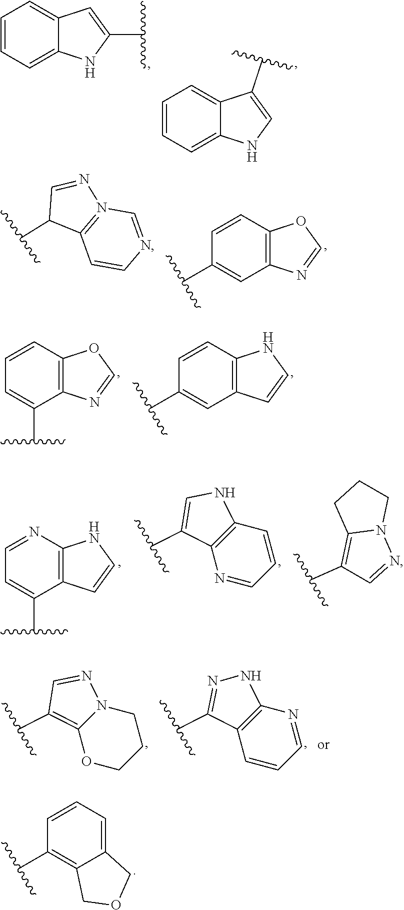

5. The compound of claim 1, or a stereoisomer, tautomer, deuterated compound, pharmaceutically acceptable salt, prodrug, chelate, non-covalent complex or solvate thereof, wherein R¹ is selected from halogen, CN, OH, —NH₂, —N(CH₃)₂, —C₁₋₆ alkyl, haloalkyl, —C₁₋₆ alkoxy, —(CH₂)ₚ(O(CH₂)_{q})ᵣCH₃, —C₃₋₆ cycloalkyl, —C₁₋₆ alkyl substituted with 3- to 7-membered heterocyclyl and 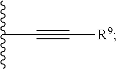
the p, q, r, s and R⁹ are defined as described in claim 1.

6. The compound of claim 5, or a stereoisomer, tautomer, deuterated compound, pharmaceutically acceptable salt, prodrug, chelate, non-covalent complex or solvate thereof, wherein R¹ is selected from halogen, CN, OH, —, —N(CH₃)₂, methyl, methoxy, —CF₃, methoxymethyl, H, oxo, —F, —Cl, —CHF₂, 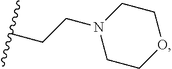 [SMILES: *CCN1CCOCC1]
ethoxy, cyclopropyl, ethyl, isopropyl, alkynyl.

7. The compound of claim 1, or a stereoisomer, tautomer, deuterated compound, pharmaceutically acceptable salt, prodrug, chelate, non-covalent complex or solvate thereof, R² is selected from H, halogen, —C₁₋₆ alkyl or —C₁₋₆ alkoxy.

8. The compound of claim 7, or a stereoisomer, tautomer, deuterated compound, pharmaceutically acceptable salt, prodrug, chelate, non-covalent complex or solvate thereof, wherein R² is selected from H, —F, methyl or methoxy.

9. The compound of claim 1, or a stereoisomer, tautomer, deuterated compound, pharmaceutically acceptable salt, prodrug, chelate, non-covalent complex or solvate thereof, wherein R³, R⁴ and R⁷ are independently selected from H, halogen, CN, OH, —C₁₋₆ alkyl, —C₁₋₆ alkoxy and —C₃₋₆ cycloalkyl; the —C₁₋₆ alkyl, —C₁₋₆ alkoxy and —C₃₋₆ cycloalkyl are optionally substituted or unsubstituted by halogen, OH, NH₂, —C₁₋₆ alkyl; R³, R⁴ and R⁷ are not H at the same time.

10. The compound of claim 9, or a stereoisomer, tautomer, deuterated compound, pharmaceutically acceptable salt, prodrug, chelate, non-covalent complex or solvate thereof, wherein R³, R⁴ and R⁷ are independently selected from H, halogen, CN, OH, methyl, ethyl, propyl, isopropyl, methoxy, ethoxy, —OCH₂CF₃ and cyclic propyl; and R³, R⁴ and R⁷ are not H at the same time.

11. The compound of claim 10, or a stereoisomer, tautomer, deuterated compound, pharmaceutically acceptable salt, prodrug, chelate, non-covalent complex or solvate thereof, wherein R³ is selected from H, methoxy.

12. The compound of claim 10, or a stereoisomer, tautomer, deuterated compound, pharmaceutically acceptable salt, prodrug, chelate, non-covalent complex or solvate thereof, wherein R⁴ is selected from H, methoxy, ethoxy or —OCH₂CF₃.

13. The compound of claim 10, or a stereoisomer, tautomer, deuterated compound, pharmaceutically acceptable salt, prodrug, chelate, non-covalent complex or solvate thereof, wherein R⁷ is selected from H, methyl or cyclopropyl.

14. The compound of claim 1, or a stereoisomer, tautomer, deuterated compound, pharmaceutically acceptable salt, prodrug, chelate, non-covalent complex or solvate thereof, wherein R⁵ is selected from —NR¹²R¹³, —C₁₋₆ alkyl, aryl, heteroaryl, —C₁₋₆ alkoxy, —C₃₋₆ cycloalkyl, 3- to 6-membered heterocyclyl, —(CH₂)ₛCH═CHR¹⁴; wherein the —C₁₋₆ alkyl, aryl, heteroaryl, —C₁₋₆ alkoxy, —C₃₋₆ cycloalkyl and 3- to 6-membered heterocyclyl are optionally substituted or unsubstituted with halogen, OH, NH₂, —C₁₋₆ alkoxy, —C₃₋₆ cycloalkyl, substituted C₃₋₆ cycloalkyl, 3- to 6-membered heterocyclyl and substituted 3- to 6-membered heterocyclyl, aryl and heteroaryl.

15. (canceled)

16. The compound of claim 1, or a stereoisomer, tautomer, deuterated compound, pharmaceutically acceptable salt, prodrug, chelate, non-covalent complex or solvate thereof, wherein R⁵ is

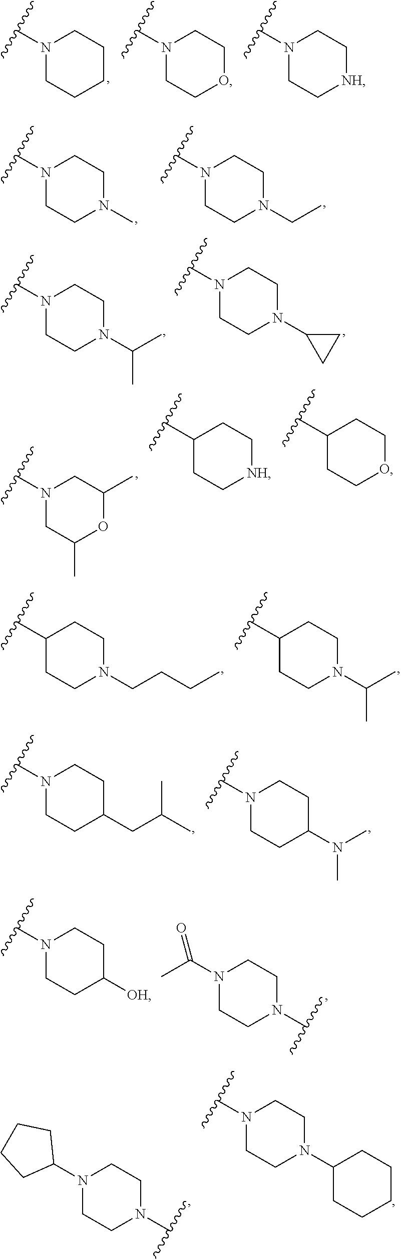

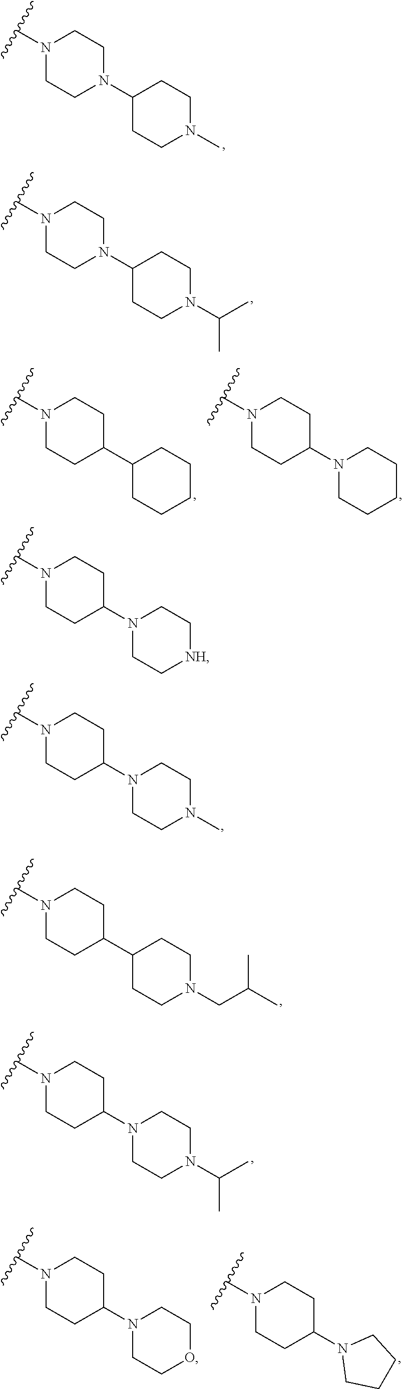

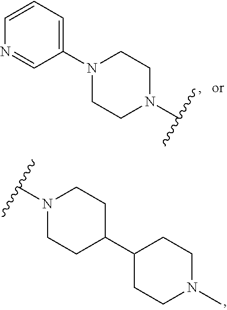 [SMILES: *N1CCN(c2cccnc2)CC1 ; *N1CCC(C2CCN(C)CC2)CC1]

17. The compound of claim 1, or a stereoisomer, tautomer, deuterated compound, pharmaceutically acceptable salt, prodrug, chelate, non-covalent complex or solvate thereof, wherein R⁶ is selected from H, halogen, CN, NO₂, OH, —C₁₋₆ alkyl, aryl, heteroaryl, 3- to 6-membered heterocyclyl, —(CH₂)ₛCH═CHR¹⁴; wherein the —C₁₋₆ alkyl, aryl, heteroaryl, and 3- to 6-membered heterocyclyl are optionally substituted or not substituted with halogen, OH, NH₂, —C₁₋₆ alkoxy, —C₃₋₆ cycloalkyl, substituted C₃₋₆ cycloalkyl, 3- to 6-membered heterocyclyl and substituted 3- to 6-membered heterocyclyl, aryl, heteroaryl.

18. The compound of claim 1, or a stereoisomer, tautomer, deuterated compound, pharmaceutically acceptable salt, prodrug, chelate, non-covalent complex or solvate thereof, wherein R⁶ select from H, methyl, ethyl, CN, allyl,

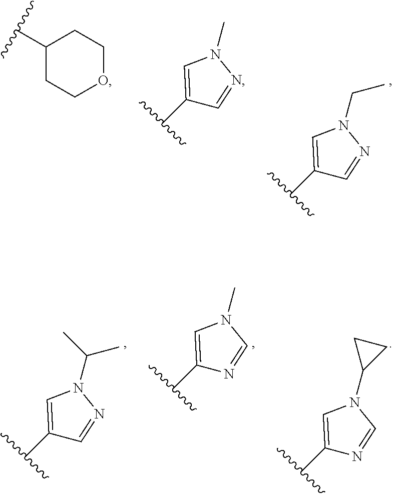

19. The compound of claim 1, or stereoisomer, tautomer, deuterated compound, pharmaceutically acceptable salt, prodrug, chelate, non-covalent complex or solvate thereof, wherein the Formula I is selected from Formula II: 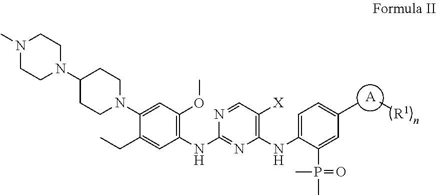
wherein,
X is halogen;
ring A is a 5- to 10-membered aromatic ring; wherein the 5- to 10-membered aromatic ring is a 5- to 10-membered aryl or a 5- to 10-membered heteroaryl;
R¹ is selected from halogen, CN, OH, —NRR⁸, oxo, —C₁₋₆ alkyl, halogenated C₁₋₆ alkyl, —C₁₋₆ alkoxy, —(CH₂)(O(CH₂)_{q})ᵣCH₃, —C₃₋₆ cycloalkyl, 3- to 6-membered heterocyclyl, —C₁₋₆ alkyl substituted with 3- to 7-membered heterocyclyl and 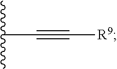
R, R⁸ and R⁹ are independently selected from H, CN, —C₁₋₆ alkyl, —C₃₋₆ cycloalkyl and 3- to 6-membered heterocyclyl, wherein —C₁₋₆ alkyl, —C₃₋₆ Cycloalkyl and 3- to 6-membered heterocyclyl are optionally substituted or unsubstituted with halogen, OH, NH₂, aryl, heteroaryl, —C₁₋₆ alkyl, —C₃₋₆ cycloalkyl, 3- to 6-membered heterocyclyl;
n is 0, 1, 2, 3 or 4;
P is 1, 2 or 3;
q is 0, 1, 2 or 3; and
r is 1, 2 or 3.

20. (canceled)

21. (canceled)

22. (canceled)

23. (canceled)

24. (canceled)

25. (canceled)

26. The compound of claim 1, or a stereoisomer, tautomer, deuterated compound, pharmaceutically acceptable salt, prodrug, chelate, non-covalent complex or solvate thereof, wherein the compound of Formula I is selected from:
(2-((5-Bromo-2-((5-ethyl-2-methoxy-4-(4-(pyrrolidin-1-yl)piperidin-1-yl)phenyl)amino)pyrimidine-4-yl)amino)-5-(1-methyl-1H-pyrazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(4-fluoro-1H-pyrazol-1-yl)phenyl)dimethylphosphine oxide;
(2-((5-Bromo-2-((2-methoxy-4-(4-(4-methylpiperazin-1-yl)piperidin-1-yl)-5-(tetrahydro-2H-pyran-4-yl)phenyl)amino)pyrimidin-4-yl)amino)-5-(thiazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(4-(methoxymethyl)-1H-pyrazol-1-yl)phenyl)dimethylphosphine oxide;
(2-((5-Bromo-2-((5-(1-ethyl-1H-pyrazol-4-yl)-2-methoxy-4-(4-(4-methylpiperazin-1-yl)piperidin-1-yl)phenyl)amino)pyrimidin-4-yl)amino)-5-(1,5-dimethyl-1H-pyrazol-3-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(2-(difluoromethyl)-2H-1,2,3-triazol-4-yl)phenyl)dimethylphosphine oxide;
(6-((5-Bromo-2-((5-ethyl-2-methoxy-4-(4-(4-methylpiperazin-1-yl)piperidin-1-yl)phenyl)amino)pyrimidin-4-yl)amino)-2-fluoro-3-(2-methyl-2H-1,2,3-triazol-4-yl)phenyl)dimethylphosphine oxide;
(2-((5-Bromo-2-((2-methoxy-5-methyl-4-(4-(4-methylpiperazin-1-yl)piperidin-1-yl)phenyl)amino)pyrimidin-4-yl)amino)-5-(2-methyl-2H-1,2,3-triazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(4-ethynyl-1H-pyrazol-1-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-ethynylpyrazin-2-yl)phenyl)dimethylphosphine oxide;
(2-((5-Bromo-2-((2-methoxy-5-methyl-4-(4-(4-methylpiperazin-1-yl)piperidin-1-yl)phenyl)amino)pyrimidin-4-yl)amino)-5-(5-ethynylpyrazin-2-yl)phenyl)dimethylphosphine oxide;
(6-((5-Bromo-2-((5-methoxy-2-methyl-4-(4-(4-methylpiperazin-1-yl)piperidin-1-yl)phenyl)amino)pyrimidin-4-yl)amino)-2-fluoro-3-(2-methyl-2H-1,2,3-triazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(6-ethynylpyrazin-2-yl)phenyl)dimethylphosphine oxide;
(2-((5-Bromo-2-((5-methoxy-2-methyl-4-(4-(4-methylpiperazin-1-yl)piperidin-1-yl)phenyl)amino)pyrimidin-4-yl)amino)-5-(5-ethynylpyrazin-2-yl)phenyl)dimethylphosphine oxide;
(2-((5-Bromo-2-((5-methoxy-2-methyl-4-morpholinophenyl)amino)pyrimidin-4-yl)amino)-5-(2-methyl-2H-1,2,3-triazol-4-yl)phenyl)dimethylphosphine oxide;
(2-((5-Bromo-2-((2-methoxy-5-methyl-4-(4-(4-methylpiperazin-1-yl)piperidin-1-yl)phenyl)amino)pyrimidin-4-yl)amino)-5-(5-ethynylthiophen-2-yl)phenyl)dimethylphosphine oxide;
(2-((5-Bromo-2-((5-methoxy-2-methyl-4-(4-(4-methylpiperazin-1-yl)piperidin-1-yl)phenyl)amino)pyrimidin-4-yl)amino)-5-(pyrazin-2-yl)phenyl)dimethylphosphine oxide;
(2-((5-Bromo-2-((5-methoxy-2-methyl-4-(4-(4-methylpiperazin-1-yl)piperidin-1-yl)phenyl)amino)pyrimidin-4-yl)amino)-5-(4-methoxy-1H-pyrazol-1-yl)phenyl)dimethylphosphine oxide;
(2-((5-Bromo-2-((5-methoxy-2-methyl-4-(4-(4-methylpiperazin-1-yl)piperidin-1-yl)phenyl)amino)pyrimidin-4-yl)amino)-5-(thiazol-2-yl)phenyl)dimethylphosphine oxide;
(2-((2-((4-([1,4′-bipiperidin]-1′-yl)-5-ethyl-2-methoxyphenyl)amino)-5-bromopyrimidine-4-yl)amino)-5-(4-methoxy-1H-pyrazol-1-yl)phenyl)dimethylphosphine oxide;
(2-((5-Bromo-2-((5-methoxy-2-methyl-4-(4-morpholinopiperidin-1-yl)phenyl)amino)pyrimidin-4-yl)amino)-5-(2-methyl-2H-1,2,3-triazol-4-yl)phenyl)dimethylphosphine oxide;
(2-((5-Bromo-2-((5-methoxy-2-methyl-4-morpholinophenyl)amino)pyrimidin-4-yl)amino)-5-(pyrazine-2-yl)phenyl)dimethylphosphine oxide;
(2-((5-Bromo-2-((5-methoxy-2-methyl-4-morpholinophenyl)amino)pyrimidin-4-yl)amino)-5-(4-methoxy-1H-pyrazol-1-yl)phenyl)dimethylphosphine oxide;
(2-((5-Bromo-2-((5-methoxy-2-methyl-4-morpholinophenyl)amino)pyrimidin-4-yl)amino)-5-(thiazol-2-yl)phenyl)dimethylphosphine oxide;
(6-((5-Bromo-2-((5-methoxy-2-methyl-4-morpholinophenyl)amino)pyrimidin-4-yl)amino)-2-fluoro-3-(2-methyl-2H-1,2,3-triazol-4-yl)phenyl)dimethylphosphine oxide;
(2-((5-Bromo-2-((2-cyclopropyl-5-methoxy-4-morpholinophenyl)amino)pyrimidin-4-yl)amino)-5-(4-methoxy-1H-pyrazol-1-yl)phenyl)dimethylphosphine oxide;
5-((5-Bromo-4-((2-(dimethylphosphine oxide)-4-(thiazol-2-yl)phenyl)amino)pyrimidin-2-yl)amino)-4-methoxy-2-morpholinobenzonitrile;
(2-((5-Bromo-2-((5-methoxy-2-methyl-4-(4-methylpiperazin-1-yl)phenyl)amino)pyrimidin-4-yl)amino)-5-(4-methoxy-1H-pyrazol-1-yl)phenyl)-dimethylphosphine oxide;
(2-((5-Bromo-2-((2-methoxy-5-methyl-4-(4-methylpiperazin-1-yl)phenyl)amino)pyrimidin-4-yl)amino)-5-(thiazol-2-yl)phenyl)dimethylphosphine oxide;
(2-((5-Bromo-2-((5-methoxy-2-methyl-4-morpholinophenyl)amino)pyrimidin-4-yl)amino)-5-(1-methyl-1H-pyrazol-4-yl)phenyl)dimethylphosphine oxide;
(2-((5-Bromo-2-((3-methoxy-5-methyl-4-morpholinophenyl)amino)pyrimidin-4-yl)amino)-5-(4-methoxy-1H-pyrazol-1-yl)phenyl)dimethylphosphine oxide;
(2-((5-Bromo-2-((5-methoxy-2-methyl-4-morpholinophenyl)amino)pyrimidin-4-yl)amino)-5-(5-methylpyridine-2-yl)phenyl)dimethylphosphine oxide;
(2-((5-Bromo-2-((5-methoxy-2-methyl-4-morpholinophenyl)amino)pyrimidin-4-yl)amino)-5-(5-cyclopropylpyrazin-2-yl)phenyl)dimethylphosphine oxide;
(6-((5-Bromo-2-((5-ethoxy-2-methyl-4-morpholinophenyl)amino)pyrimidin-4-yl)amino)-2-fluoro-3-(2-methyl-2H-1,2,3-triazol-4-yl)phenyl)dimethylphosphine oxide;
(2-((5-Bromo-2-((5-ethoxy-2-methyl-4-morpholinophenyl)amino)pyrimidin-4-yl)amino)-5-(5-methylpyridine-2-yl)phenyl)dimethylphosphine oxide;
(6-((5-Bromo-2-((2-methyl-4-morpholin-5-(2,2,2-trifluoroethyl)phenyl)amino)pyrimidin-4-yl)amino)-2-fluoro-3-(2-methyl-2H-1,2,3-triazol-4-yl)phenyl)dimethylphosphine oxide;
(6-((5-Bromo-2-((4-(2,6-dimethylmorpholine)-5-methoxy-2-methylphenyl)amino)pyrimidin-4-yl)amino)-2-fluoro-3-(2-methyl-2H-1,2,3-triazol-4-yl)phenyl)dimethylphosphine oxide;
(2-((5-Bromo-2-((4-(4-(dimethylamino)piperidin-1-yl)-5-methoxy-2-methylphenyl)amino)pyrimidine-4-yl)amino)-5-(4-methoxy-1H-pyrazol-1-yl)phenyl)dimethylphosphine oxide;
(2-((5-Bromo-2-((4-(4-hydroxypiperidin-1-yl)-5-methoxy-2-methylphenyl)amino)pyrimidin-4-yl)amino)-5-(4-Methoxy-1H-pyrazol-1-yl)phenyl)dimethylphosphine oxide;
(2-((5-Bromo-2-((5-ethyl-2-methoxy-4-(4-morpholinopiperidin-1-yl)phenyl)amino)pyrimidin-4-yl)amino)-5-(2-methyl-2H-1,2,3-triazol-4-yl)phenyl)dimethylphosphine oxide;
(6-((5-Bromo-2-((5-ethyl-2-methoxy-4-(4-(4-methylpiperazin-1-yl)piperidin-1-yl)phenyl)amino)pyrimidin-4-yl)amino)-2-methoxy-3-(1-methyl-1H-pyrazol-4-yl)phenyl)dimethylphosphine oxide;
(2-((5-Bromo-2-((5-ethyl-2-methoxy-4-(4-morpholinopiperidin-1-yl)phenyl)amino)pyrimidin-4-yl)amino)-5-(1-methyl-1H-pyrazol-4-yl)phenyl)dimethylphosphine oxide;
(2-((5-Bromo-2-((2-methoxy-4-(4-(4-methylpiperazin-1-yl)piperidin-1-yl)-5-propylphenyl)amino)pyrimidin-4-yl)amino)-5-(1-methyl-1H-pyrazol-4-yl)phenyl)dimethylphosphine oxide;
(2-((5-Bromo-2-((4-(4-(dimethylamino)piperidin-1-yl)-5-ethyl-2-methoxyphenyl)amino)pyrimidine-4-yl)amino)-5-(1-methyl-1H-pyrazol-4-yl)phenyl)dimethylphosphine oxide;
(2-((2-((5-Allyl-2-methoxy-4-(4-(4-methylpiperazin-1-yl)piperidin-1-yl)phenyl)amino)-5-bromopyrimidin-4-yl)amino)-5-(1-methyl-1H-pyrazol-4-yl)phenyl)dimethylphosphine oxide;
(6-((5-Bromo-2-((5-ethyl-2-methoxy-4-(4-(4-methylpiperazin-1-yl)piperidin-1-yl)phenyl)amino)pyrimidin-4-yl)amino)-2-methyl-3-(1-methyl-1H-pyrazol-4-yl)phenyl)dimethylphosphine oxide;
(4-((5-Bromo-2-((5-ethyl-2-methoxy-4-(4-morpholinopiperidin-1-yl)phenyl)amino)pyrimidin-4-yl)amino)-[1,1′-biphenyl]-3-yl)dimethylphosphine oxide;
(4-((5-Bromo-2-((5-ethyl-2-methoxy-4-(4-morpholinopiperidin-1-yl)phenyl)amino)pyrimidin-4-yl)amino)-2′-fluoro-[1,1′-biphenyl]-3-yl)dimethylphosphine oxide;
(4-((5-Bromo-2-((5-ethyl-2-methoxy-4-(4-morpholinopiperidin-1-yl)phenyl)amino)pyrimidin-4-yl)amino)-2′-methyl-[1,1′-biphenyl]-3-yl)dimethylphosphine oxide;
(4-((5-Bromo-2-((5-ethyl-2-methoxy-4-(4-(4-methylpiperazin-1-yl)piperidin-1-yl)phenyl)amino)pyrimidin-4-yl)amino)-2-methyl-[1,1′-biphenyl]-3-yl)dimethylphosphine oxide; and
(4-((2-((4-([1,4′-bipiperidin]-1′-yl)-2-methoxy-5-(1-methyl-1H-pyrazole-4-yl)phenyl)amino)-5-bromopyrimidin-4-yl)amino)-2-methyl-[1,1′-biphenyl]-3-yl)dimethylphosphine oxide;
(5-(Benzo[d]oxazol-4-yl)-2-((5-chloro-2-((5-ethyl-2-methoxy-4-(4-(4-methylpiperazin-1-yl)piperidin-1-yl)phenyl)amino)pyrimidin-4-yl)amino)phenyl)dimethylphosphine oxide;
(5-(2-Aminothiazol-4-yl)-2-((5-chloro-2-((5-ethyl-2-methoxy-4-(4-(4-methylpiperazin-1-yl)piperidin-1-yl)phenyl)amino)pyrimidin-4-yl)amino)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(4-methoxy-1H-pyrazol-1-yl)phenyl)dimethylphosphine oxide;
(5-(3-Aminopyridin-2-yl)-2-((5-chloro-2-((5-ethyl-2-methoxy-4-(4-(4-methylpiperazin-1-yl)piperidin-1-yl)phenyl)amino)pyrimidin-4-yl)amino)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-isopropylpyrimidin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(2-(dimethylamino)pyrimidin-5-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-ethoxypyrimidin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(2-ethoxypyrimidin-5-yl)phenyl)dimethylphosphine oxide;
3-(4-((5-Chloro-2-((5-ethyl-2-methoxy-4-(4-(4-methylpiperazin-1-yl)piperidin-1-yl)phenyl)amino)pyrimidin-4-yl)amino)-3-(dimethylphosphine oxide)phenyl)-1-methyl-1H-pyrazol-4-carbonitrile;
(2-((5-Chloro-2-((5-ethyl-2-methoxy-4-(4-(4-methylpiperazin-1-yl)piperidin-1-yl)phenyl)amino)pyrimidin-4-yl)amino)-5-(3-ethoxy-5-methylisoxazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(2-ethyl-2H-1,2,3-triazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(pyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-cyclopropylpyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-methylpyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(1,5-dimethyl-1H-pyrazol-3-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5,6-dimethylpyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(2-isopropyl-2H-1,2,3-triazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(4-methyl-1H-pyrazol-1-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(6-methylpyridin-3-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-methylpyridin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-methoxythiophen-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(4-chlorothiazol-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-methoxypyridin-2-yl)phenyl)dimethylphosphine oxide;
(6-((5-Bromo-2-((5-methoxy-2-methyl-4-(4-(4-methylpiperazin-1-yl)piperidin-1-yl)phenyl)amino)pyrimidin-4-yl)amino)-2-fluoro-3-(4-methoxy-1H-pyrazol-1-yl)phenyl)dimethylphosphine oxide;
(2-((5-Bromo-2-((2-methoxy-5-methyl-4-(4-(4-methylpiperazin-1-yl)piperidin-1-yl)phenyl)amino)pyrimidin-4-yl)amino)-5-(4-methoxy-1H-pyrazol-1-yl)phenyl)dimethylphosphine oxide;
(6-((5-Bromo-2-((2-methoxy-5-methyl-4-(4-(4-methylpiperazin-1-yl)piperidin-1-yl)phenyl)amino)pyrimidin-4-yl)amino)-2-fluoro-3-(4-fluoro-1H-pyrazol-1-yl)phenyl)dimethylphosphine oxide;
(4-((5-Bromo-2-((5-ethyl-2-methoxy-4-(morpholinopiperidin-1-yl)phenyl)amino)pyrimidin-4-yl)amino)-2′-methyl-[1,1′-diphenyl]-3-yl)dimethylphosphine oxide;
(2-((5-Bromo-2-((4-(4-(dimethylamino)piperidin-1-yl)-5-ethyl-2-methoxyphenyl)amino)pyrimidine-4-yl)amino)-5-(1-methyl-1H-pyrazolo[3,4-b]pyridin-3-yl)phenyl)dimethylphosphine oxide;
(2-((5-Bromo-2-((5-(1-ethyl-1H-pyrazol-4-yl)-2-methoxy-4-(4-(4-methylpiperazin-1-yl)piperidin-1-yl)phenyl)amino)pyrimidin-4-yl)amino)-5-(1,5-dimethyl-1H-pyrazol-3-yl)phenyl)dimethylphosphine oxide;
(2-((5-Bromo-2-((5-((4-(4-cyclopentylpiperazin-1-yl)-2-methoxy-5-(1-methyl-1H-Pyrazol-4-yl)phenyl)amino)pyrimidin)-4-yl)amino)-5-(4-methoxy-1H-pyrazol-1-yl)phenyl)dimethylphosphine oxide;
1-(4-(4-((5-Bromo-4-((2-(dimethylphosphate)-4-(4-methoxy-1H-pyrazol-1-yl)phenyl)amino)pyrimidin)-2-yl)amino)-5-methoxy-2-(1-methyl-1H-pyrazol-4-yl)phenyl)piperazin-1-yl-acetylacetone;
(2-((5-Bromo-2-((2-methoxy-5-(1-methyl-1H-pyrazol-4-yl)-4-(4-(pyridin-3-yl)piperazin-1-yl)phenyl)amino)pyrimidin-4-yl)amino)-5-(4-methoxy-1H-imidazol-1-yl)phenyl)dimethylphosphine oxide;
(2-((5-Bromo-2-((5-methoxy-2-methyl-4-(4-(4-methylpiperazin-1-yl)piperidin-1-yl)phenyl]amino]amino]pyrimidin-4-yl)amino)-5-(4-(trifluoromethyl)-1H-pyrazol-1-yl)phenyl)dimethylphosphine oxide;
(2-((5-Bromo-2-((2-methoxy-5-(1-methyl-1H-pyrazol-4-yl)-4-(4-(4-methylpiperazin-1-yl)piperidin-1-yl)phenyl)amino)pyrimidin-4-yl)amino)-5-(4-(trifluoromethyl)-1H-pyrazol-1-yl)phenyl)dimethylphosphine oxide;
(2-((5-Bromo-2-((2-methoxy-5-methyl-4-(4-(4-methylpiperazin-1-yl)piperidin-1-yl)phenyl)amino]amino]pyrimidin-4-yl)amino)-5-(4-(trifluoromethyl)-1H-pyrazol-1-yl)phenyl)dimethylphosphine oxide;
(2-((2-((4-([[1,4′-bipiperidin]-1-1-yl)-2-methoxy-5-methylphenyl)amino)-5-bromopyrimidin-4-yl)amino)-5-(4-methoxy-1H-pyrazol-1-yl)phenyl)dimethylphosphine oxide;
(2-((5-Bromo-2-((2-methoxy-5-methyl-4-(4-(1-methylpiperidin-4-yl)piperazin-1-yl)phenyl)amino]amino]pyrimidin-4-yl)amino)-5-(4-methoxy-1H-pyrazol-1-yl)phenyl)dimethylphosphine oxide;
(2-((5-Bromo-2-((4-(4-cyclopentylpiperazin-1-yl)-2-methoxy-5-methylphenyl)amino)pyrimidin-4-yl)amino)-5-(4-methoxy-1H-pyrazol-1-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(4-(methoxymethyl)-1H-pyrazol-1-yl)phenyl)dimethylphosphine oxide;
(2-((5-Bromo-2-((5-(1-ethyl-1H-pyrazol-4-yl)-2-methoxy-4-(4-(4-methylpiperazin-1-yl)piperidin-1-yl)phenyl)amino)pyrimidin-4-yl)amino)-5-(1H-indol-7-yl)phenyl)dimethylphosphine oxide;
3-(4-((5-Chloro-2-((5-ethyl-2-methoxy-4-(4-(4-methylpiperazin-1-yl)piperidin-1-yl)phenyl)amino)pyrimidin-4-yl)amino)-3-(dimethylphosphoryl)phenyl)pyridin-2(1H)-quinoxalinone;
(4-((5-Bromo-2-((5-ethyl-2-methoxy-4-(4-morpholinopiperidin-1-yl)phenyl)amino)pyrimidin-4-yl)amino)-2′-fluoro-[1,1′-biphenyl]-3-yl)dimethylphosphine oxide;
(2-((5-Bromo-2-((5-ethyl-2-methoxy-4-(4-morpholinopiperidin-1-yl)phenyl)amino)pyrimidin-4-yl)amino)-5-(2-methyl-2H-1,2,3-triazol-4-yl)phenyl)dimethylphosphine oxide;
(5-(2-Aminothiazol-4-yl)-2-((5-chloro-2-((5-ethyl-2-methoxy-4-(4-(4-methylpiperazin-1-yl)piperidin-1-yl)phenyl)amino)pyrimidin-4-yl)amino)phenyl)dimethylphosphine oxide;
(2-((5-Bromo-2-((2-methoxy-4-(4-(4-methylpiperazin-1-yl)piperidin-1-yl)-5-(tetrahydro-2H-pyran-4-yl)phenyl)amino)pyrimidin-4-yl)amino)-5-(thiazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(6-hydroxypyridazin-3-yl)phenyl)dimethylphosphine oxide;
(5-(3-Aminopyridin-2-yl)-2-((5-bromo-2-((5-ethyl-2-methoxy-4-(4-(4-methylpiperazin-1-yl)piperidin-1-yl)phenyl)amino)pyrimidin-4-yl)amino)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(4-hydroxypyrimidin-5-yl)phenyl)dimethylphosphine oxide;
(4-((2-((4-(4-([1,4′-bipiperidin]-1′-yl)-2-methoxy-5-(1-methyl-1H-pyrazole)-4-yl)phenyl)amino)-5-bromopyrimidin-4-yl)amino)-[1,1′-biphenyl]-3-yl)dimethylphosphine oxide;
(2-((5-Bromo-2-((5-ethyl-2-methoxy-4-(4-(4-methylpiperazin-1-yl)piperidin-1-yl)phenyl)aminopyrimidin-4-yl)amino)-5-(1,5-dimethyl-1H-pyrazol-3-yl)phenyl dimethylphosphine oxide;
(2-((5-Bromo-2-((5-(1-ethyl-1H-pyrazol-4-yl)-2-methoxy-4-(4-(4-methylpiperazin-1-yl)piperidin-1-yl)phenyl)amino)pyrimidin-4-yl)amino)-5-(1-methyl-1H-pyrazolo[3,4-b]pyridin-3-yl)phenyl)dimethylphosphine oxide;
(5-(3-Aminopyridin-2-yl)-2-((5-chloro-2-((5-ethyl-2-methoxy-4-(4-(4-methylpiperazin-1-yl)piperidin-1-yl)phenyl)amino)pyrimidin-4-yl)amino)phenyl)dimethylphosphine oxide;
2-((5-Chloro-2-((5-ethyl-2-methoxy-4-(4-(4-methylpiperazin-1-yl)piperidin-1-yl)phenyl)amino)pyrimidin-4-yl)amino)-5-(4,5,6,7-tetrahydropyrazolo[1,5-a]pyridin-3-yl)phenyl)dimethylphosphine oxide;
(4-((5-Bromo-2-((5-ethyl-2-methoxy-4-(4-morpholinopiperidin-1-yl)phenyl)amino)pyrimidin-4-yl)amino)-[1,1′-biphenyl]-3-yl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(2-(difluoromethyl)-2H-1,2,3-triazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(1H-pyrazol-5-yl)phenyl)dimethylphosphine oxide;
(2-((5-Chloro-2-((5-ethyl-2-methoxy-4-(4-(4-methylpiperazin-1-yl)piperidin-1-yl)phenyl)amino)pyrimidin-4-yl)amino)-5-(1-methyl-1-imidazol-2-yl)phenyl)dimethylphosphine oxide;
(2-((5-Chloro-2-((5-ethyl-2-methoxy-4-(4-(4-methylpiperazin-1-yl)piperidin-1-yl)phenyl)amino)pyrimidin-4-yl)amino)-5-(5,5-dimethyl-5,6-dihydro-tetrahydro-pyrrole[1,2-b]pyrazol-3-)phenyl)dimethylphosphine oxide;
(2-((5-Bromo-2-((5-ethyl-2-methoxy-4-(-4-(4-methylpiperazin-1-yl)piperidin-1-yl)phenyl)amino)pyrimidin-4-yl)amino)-5-(1-methyl-1H-pyrrolo[3,2-b]pyridin-3-yl)phenyl)dimethylphosphine oxide;
2-(4-((5-Bromo-2-((5-ethyl-2-methoxy-4-(4-(4-methylpiperazin-1-yl)piperidin-1-yl)Phenyl)amino)pyrimidin-4-yl)amino)-3-(dimethylphosphino)phenyl)-1H-indole-5-carbonitrile;
(2-((5-Bromo-2-((5-ethyl-2-methoxy-4-(4-(4-methylpiperazin-1-yl)piperidin-1-yl)phenyl)amino)pyrimidin-4-yl)amino)-5-(4-fluoro-1H-pyrazol-1-yl)phenyl)dimethylphosphine oxide;
(2-((5-Chloro-2-((5-ethyl-2-methoxy-4-(4-(4-methylpiperazin-1-yl)piperidin-1-yl)phenyl)amino)pyrimidin-4-yl)amino)-5-(5-methyl-1,3,4-oxadiazol-2-yl)phenyl)dimethylphosphine oxide;
(2-((5-Bromo-2-((5-ethyl-2-methoxy-4-(-4-(4-methylpiperazin-1-yl)piperidin-1-yl)phenyl)amino)pyrimidin-4-yl)amino)-5-(1-methyl-1H-pyrrolo[2,3-b]pyridin-3-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(1-(2-morpholinoethyl)-1H-pyrazol-4-yl)phenyl)dimethylphosphine oxide;
(2-((5-Chloro-2-((5-ethyl-2-methoxy-4-(4-(4-methylpiperazin-1-yl)piperidin-1-yl)phenyl)amino)pyrimidin-4-yl)amino)-5-(1H-pyrazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(1H-pyrazol-1-yl)phenyl)dimethylphosphine oxide;
(2-((5-Bromo-2-((5-ethyl-2-methoxy-4-(4-(4-methylpiperazin-1-yl)piperidin-1-yl)phenyl)amino]amino]pyrimidin-4-yl)amino)-5-(4-methoxy-1H-pyrazol-1-yl)phenyl)dimethylphosphine oxide;
4′-((5-Bromo-2-((5-ethyl-2-methoxy-4-(4-(4-methylpiperazin-1-yl)piperidin-1-yl)phenyl)amino)pyrimidin-4-yl)amino)-3′-(dimethylphosphoryl)-[1,1′-biphenyl]-2-carbonitrile;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(1H-indol-7-yl)phenyl)dimethylphosphine oxide;
(2-((5-Chloro-2-((5-ethyl-2-methoxy-4-(4-(4-methylpiperazin-1-yl)piperidin-1-yl)phenyl)amino)pyrimidin-4-yl)amino)-5-(4-methoxypyridin-3-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(1,4-dimethyl-1H-pyrazol-5-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(1-methyl-1H-pyrrolo[2,3-b]pyridin-5-yl)phenyl)dimethylphosphine oxide;
(2-((5-Chloro-2-((5-ethyl-2-methoxy-4-(4-(4-methylpiperazin-1-yl)piperidin-1-yl)phenyl)amino)pyrimidin-4-yl)amino)-5-(1-methyl-1H-pyrrolo[2,3-b]pyridin-4-yl)phenyl)dimethylphosphine oxide;
3-(4-((5-Chloro-2-((5-ethyl-2-methoxy-4-(4-(4-methylpiperazin-1-yl)piperidin-1-yl)phenyl)amino)pyrimidin-4-yl)amino)-3-(dimethylphosphoryl)phenyl)-1-methyl-1H-pyrazol-4-carbonitrile;
(2-((5-Chloro-2-((5-ethyl-2-methoxy-4-(4-(4-methylpiperazin-1-yl)piperidin-1-yl)phenyl)amino)pyrimidin-4-yl)amino)-5-(3-ethoxy-5-methylisoxazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(6-methoxypyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(5-methylthiazol-4-yl)phenyl)dimethylphosphine oxide;
(5-(Benzo[d]oxazol-4-yl)-2-((5-chloro-2-((5-ethyl-2-methoxy-4-(4-(4-methylpiperazin-1-yl)piperidin-1-yl)phenyl)amino)pyrimidin-4-yl)amino)phenyl)dimethylphosphine oxide;
(2-((5-Bromo-2-((5-ethyl-2-methoxy-4-(4-(4-methylpiperazin-1-yl)piperidin-1-yl)phenyl)amino]pyrimidin-4-yl)amino)-5-(1-methyl-1H-1,2,4-triazol-5-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(3-methoxy-1-methyl-1H-pyrazol-4-yl)phenyl)dimethylphosphine oxide;
4-(4-((5-Bromo-2-((5-ethyl-2-methoxy-4-(4-(4-methylpiperazin-1-yl)piperidin-1-yl)phenyl)amino]pyrimidin-4-yl)amino)-3-(dimethylphosphoryl)phenyl)-1-methyl-1H-pyrazol-3-carbonitrile;
(2-((5-Chloro-2-((5-ethyl-2-methoxy-4-(4-(4-methylpiperazin-1-yl)piperidin-1-yl)phenyl)amino)pyrimidin-4-yl)amino)-5-(4-methoxypyrimidin-5-yl)phenyl)dimethylphosphine oxide;
(2-((5-Chloro-2-((5-ethyl-2-methoxy-4-(4-(4-methylpiperazin-1-yl)piperidin-1-yl)phenyl)amino)pyrimidin-4-yl)amino)-5-(4-methylpyrimidin-5-yl)phenyl)dimethylphosphine oxide;
(2-((5-Chloro-2-((5-ethyl-2-methoxy-4-(4-(4-methylpiperazin-1-yl)piperidin-1-yl)phenyl)amino)pyrimidin-4-yl)amino)-5-(2-methylpyrimidin-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(1-methyl-1H-imidazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(1-cyclopropyl-1H-imidazol-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-ethylpyrimidin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(6-(dimethylamino)pyridin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(3-methoxypyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-isopropylpyrimidin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(2-(dimethylamino)pyrimidin-5-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(5-ethoxypyrimidin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)amino]pyrimidin-4-yl)amino)-5-(2-ethoxypyrimidin-5-yl)phenyl)dimethylphosphine oxide;
(2-((5-Bromo-2-((5-ethyl-2-methoxy-4-(4-(4-methylpiperazin-1-yl)piperidin-1-yl)phenyl)amino]pyrimidin-4-yl)amino)-5-(6-(dimethylamino)pyridazin-3-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(pyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-cyclopropylpyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-methylpyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5,6-dimethylpyrazin-2-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(6-methylpyridin-3-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(5-methylpyridin-2-yl)phenyl)dimethylphosphine oxide;
(6-((5-Bromo-2-((5-ethyl-2-methoxy-4-(4-(4-methylpiperazin-1-yl)piperidin-1-yl)phenyl)amino)pyrimidin-4-yl)amino)-2-fluoro-3-(2-methyl-2H-1,2,3-triazol-4-yl)phenyl)dimethylphosphine oxide;
(2-((5-Bromo-2-((5-ethyl-2-methoxy-4-(4-(4-methylpiperazin-1-yl)piperidin-1-yl)phenyl)amino)pyrimidin-4-yl)amino)-5-(2-isopropyl-2H-1,2,3-triazol-4-yl)phenyl)dimethylphosphine oxide; and
(2-((5-Bromo-2-((5-ethyl-2-methoxy-4-(4-(4-methylpiperazin-1-yl)piperidin-1-yl)phenyl)amino)pyrimidin-4-yl)amino)-5-(4-methyl-1H-pyrazol-1-yl)phenyl)dimethylphosphine oxide.

27. A pharmaceutical composition, wherein, the pharmaceutical composition includes a compound of claim 1, or its stereoisomer, tautomer, deuterated compound, pharmaceutically acceptable salt, prodrug, chelate, non-covalent complex or solvate as the active component, and at least one pharmaceutically acceptable adjuvant.

28. (canceled)

29. A method for treating EGFR-driven cancers, wherein, the method comprises administering to a patient in need thereof a therapeutically effective amount of a compound of claim 1, or a stereoisomer, tautomer, deuterated compound, pharmaceutically acceptable salt, prodrug, chelate, non-covalent complex or solvate thereof.

30. The method for treating EGFR-driven cancers of claim 29, wherein the EGFR-driven cancer is characterized by the presence of one or more mutations from: (i) C797S, (ii) L858R and C797S, (iii) C797S and T790M, (iv) L858R, T790M and C797S, or (v) Δ19del, T790M and C797S.

31. The method for treating EGFR-driven cancers of claim 29, wherein the EGFR-driven cancers include colon cancer, stomach cancer, thyroid cancer, lung cancer, leukemia, pancreatic cancer, melanoma, brain cancer, kidney cancer, prostate cancer, ovarian cancer, or breast cancer.

32. The method for treating EGFR-driven cancers of claim 31, wherein the lung cancer is non-small cell lung cancer caused by EGFR^{L85R/T790M/C797S} or EGFR^{Δ19del/T790M/C797S} mutant.

33-37. (canceled)
